(19) United States
(12) Patent Application Publication
(10) Pub. No.: US 2022/0324865 A1
(43) Pub. Date: Oct. 13, 2022

(54) SUBSTITUTED DIHYDROPYRAZOLO PYRAZINE CARBOXAMIDE DERIVATIVES

(71) Applicants: Bayer Aktiengesellschaft, Leverkusen (DE); Bayer Pharma Aktiengesellschaft, Berlin (DE)

(72) Inventors: Steffen MUELLER, Muelheim an der Ruhr (DE); Rudolf SCHOHE-LOOP, Wuppertal (DE); Nuria ORTEGA HERNANDEZ, Wuppertal (DE); Frank SUESSMEIER, Muenchen (DE); Eloisa JIMENEZ NUNEZ, Freiburg im Breisgau (DE); Thomas BRUMBY, Berlin (DE); Niels LINDNER, Wuppertal (DE); Christoph GERDES, Koeln (DE); Elisabeth POOK, Wuppertal (DE); Anja BUCHMUELLER, Essen (DE); Fabienne Zdenka GAUGAZ, Uppsala (SE); Dieter LANG, Velbert (DE); Stefanie ZIMMERMANN, Duesseldorf (DE); Alexander Helmut Michael EHRMANN, Waltham, MA (US); Michael GERISCH, Wuppertal (DE); Lutz LEHMANN, Berlin (DE); Andreas TIMMERMANN, Duesseldorf (DE); Martina SCHAEFER, Berlin (DE); Georg SCHMIDT, Wuppertal (DE); Karl-Heinz SCHLEMMER, Wupptertal (DE); Markus FOLLMANN, Koeln (DE); Elisabeth KERSTEN, Wuppertal (DE); Vivian WANG, Beijing (CN); Xiang GAO, Beijing (CN); Yafeng WANG, Beijing (CN)

(21) Appl. No.: 17/055,190

(22) PCT Filed: May 10, 2019
(86) PCT No.: PCT/EP2019/062005

§ 371 Date: Nov. 13, 2020

(30) Foreign Application Priority Data
May 17, 2018 (CN) PCT/CN2018/087249

(51) Int. Cl. C07D 487/04; A61P 7/02

(52) U.S. Cl. CPC C07D 487/04; A61P 7/02

(57) ABSTRACT

The invention relates to substituted dihydropyrazolo pyrazine carboxamide derivatives and to processes for their preparation, and also to their use for preparing medicaments for the treatment and/or prophylaxis of diseases, in particular cardiovascular disorders, preferably thrombotic or thromboembolic disorders, and diabetes, and also urogenital and ophthalmic disorders.

The invention relates to substituted dihydropyrazolo pyrazine carboxamide derivatives and to processes for their preparation, and also to their use for preparing medicaments for the treatment and/or prophylaxis of diseases, in particular cardiovascular disorders, preferably thrombotic or thromboembolic disorders, and diabetes, and also urogenital and ophthalmic disorders.

Atherothrombosis is the main complication of atherosclerosis and underlies several of the most lethal human diseases, such as myocardial infarction (MI), ischemic stroke (IS) and peripheral arterial occlusive disease (PAOD). The process is initiated by the deposition of lipids and their subsequent oxidation in the arterial wall which induces the recruitment of inflammatory cells and the formation of atherosclerotic plaques. These plaques are covered by a fibrous cap which maintains the plaque content separated from the blood flow. Inside the plaque, pro-inflammatory mechanisms drive inflammatory cells to produce matrix metalloproteases which digest the proteins of the fibrous cap. The thinned cap is called “vulnerable”, meaning that the cap may rupture relatively easily in response to stresses. When the cap ruptures or its endothelial cover erodes, the plaque content comes into contact with the blood and provokes the formation of an intravascular thrombus by activating platelets and blood coagulation. This process, forming a thrombus on plaques, is called atherothrombosis. If obstructive, the resulting intravascular thrombus interrupts blood flow and causes ischemia of downstream tissues with dramatic clinical consequences representing the leading cause of death and morbidity worldwide.

Given the mechanism of atherothrombotic vessel obstruction, current prevention targets the mechanisms of thrombosis, which is the pathological formation of intra-vascular plugs. The mechanisms of thrombosis encompass two intertwined pathways, the coagulation cascade and the aggregation of platelets, which once activated by a vessel injury act synergistically to build the intravascular clot obstructing the vessel lumen. Anticoagulant and anti-aggregant drugs, used to prevent atherothrombosis, have elicited a considerable reduction of the rate of second myocardial infarctions and a decrease of long term mortality from about 30% prior to the 1980s to less than 10% after the 2000s (Arch. Intern. Med. 2002, 162, 2405-2410; Lancet 2011, 377, 2193-2204). Specifically, the COX inhibitor Aspirin and the ADP receptor P2Y12 antagonist Clopidogrel are components of dual antiplatelet therapy. Prasugrel and Ticagrelor are alternative P2Y12 blockers that have demonstrated reductions of cardiovascular ischemic events (N. Engl. J. Med. 2007, 357, 2001-2015; ibid. 2009, 361, 1045-1057). The coagulation factor Xa inhibitor Rivaroxaban has also shown benefits to patients with stable atherosclerotic vascular disease (N. Engl. J. Med. 2017, 377, 1319-1330). However, activation of platelets and blood coagulation is also crucial for hemostasis to prevent excessive blood loss after vascular injury. Consequently, currently marketed antiplatelet drugs and anticoagulants achieve their therapeutic effect at the cost of increased bleeding rates. Therefore, a safe antithrombotic drug must preserve the competence of hemostasis.

An approach to widen the therapeutic window between antithrombotic-effective dose and the hemostasis-compromising dose is to target mechanisms which are restricted to the site of atherosclerotic vessel walls. It has been widely shown that plaques host and maintain inflammation. A prothrombotic mediator produced by inflammatory mechanisms within plaques, but not by healthy vascular walls, would be an ideal target to impact atherothrombosis only at the site of the plaque without an impact on systemic hemostasis. Among other factors, local synthesis of prostanoids from arachidonic acid (AA) in the arterial vessel wall may play a profound role in atherosclerosis. Besides TXA2, the AA pathway generates several other mediators, e.g. prostaglandin E2 (PGE2). Increased PGE2 concentrations have been measured in atherosclerotic vascular walls of mice and humans [Circulation 2001, 104, 921-927; J. Exp. Med. 2007, 204, 311-320; Cardiovasc. Res. 2014, 101, 482-491]. Once released upon plaque rupture, PGE2 binds on four specific receptors EP1, EP2, EP3 and EP4 on cell membranes. PGE2 has been shown to interfere with human and murine platelet function via EP3 and EP4 receptors (Eur. J. Pharmacol. 1991, 194, 63-70). Stimulation of EP3 potentiates platelet activation and aggregation induced by primary agonists like collagen or ADP, whereas stimulation of EP4 inhibits platelet activation. This PGE2-dependent balance of platelet activation and inhibition can be tipped by modulation of EP3 or EP4 receptors (Platelets 2010, 21, 329-342; Prostaglandins & Other Lipid Mediators 2015, 121, 4-16).

Therefore, blocking the EP3 receptor by specific antagonists should be a beneficial strategy for prevention and treatment of atherothrombosis by local abrogation of platelet activation without altering hemostasis.

In patients suffering from PAOD, chronically inflamed vessel walls produce PGE2 to activate EP3 receptors not only on platelets but also on vascular smooth muscle cells thus preventing microvascular relaxation and contributing to malperfusion of peripheral tissues. Therefore, an antagonist to the EP3 receptor might be expected to provide therapeutic benefit specifically in PAOD.

Furthermore, PGE2 originating from inflammatory processes has been shown to compromise glucose-stimulated insulin secretion from pancreatic beta cells via the EP3 receptor pathway (Diabetes 2013, 62, 1904-1912). Therefore, EP3 antagonists might represent a beneficial strategy in the treatment of patients with type II diabetes.

In the kidney and urogenital tract, PGE2 participates in the regulation of renal microcirculation, diuresis and bladder excitability. EP3 receptor antagonists might help to improve renal disorders and in particular to resolve bladder hyperactivity.

Furthermore, for many disorders the combination of antithrombotic and anti-inflammatory principles may also be particularly attractive to prevent the mutual enhancement of coagulation and platelet activation.

In the field of ophthalmology, prostaglandin derivatives participate in the regulation of intraocular pressure and inflammation. Therefore, compounds modulating the respective receptors may have a benefit in the prevention and treatment of ocular diseases.

Certain pyrazolo pyrazine derivatives are known having different pharmaceutical activity and are useful e.g. as autotaxin inhibitors or inhibitors of histone demethylases, or for the treatment of cancer, cardiovascular diseases, viral infections and as herbicides (cf. WO 2015/129821, WO 2014/139326, WO 2013/143663, WO 2009/082687, WO 2009/023179, WO 2006/050803, WO 2005/120516, and WO 2017/144995).

WO 2015/052610 and WO 2016/103097 provide antagonists of prostaglandin EP3 receptor having a pyridinone substituted indazole, indole or quinoline derivative. Amide or pyridinone based EP3 receptor antagonists are also described in ACS Med. Chem. Lett. 2010, 1, 316-320 and in Bioorg. Med. Chem. Lett. 2009, 19, 4292-4295, ibid. 2011, 21, 2806-2811, ibid. 2016, 26, 2670-2675.

It is therefore an object of the present invention to provide novel compounds for the treatment of cardiovascular disorders, in particular of thrombotic or thromboembolic disorders, in humans and animals, which compounds have a wide therapeutic window and, in addition, a good pharmaco-kinetic behavior.

Surprisingly, it has now been found that certain substituted dihydropyrazolo pyrazine carboxamide derivatives represent highly potent antagonists of prostaglandin EP3 receptor.

The invention provides compounds of the formula (I)

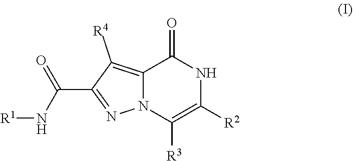

in which
R¹ represents C₁-C₆-alkyl, C₂-C₆-halogenoalkyl, C₃-C₆-cycloalkyl or the group -L-R^{E},
   where alkyl may be substituted by 1 or 2 substituents independently selected from the group consisting of hydroxyl, cyano, cyclopropyl, cyclobutyl, azetidin-1-yl (which may be substituted by 1 or 2 fluorine atoms), methoxy, methylsulfonyl, carbamoyl, NR^{a}R^{b} (where R^{a} and R^{b} are independently selected from the group consisting of hydrogen, C₁-C₄-alkyl, C₂-C₆-halogeno-alkyl or cyclopropyl, or where R^{a} and R^{b} together with the nitrogen atom to which they are bound may form a morpholine ring) and C₁-C₃-halogenoalkoxy, where halogenoalkoxy is substituted by 1 to 3 fluorine atoms,
   and
   where halogenoalkyl is substituted by 1 to 6 fluorine atoms and may be further substituted by 1 or 2 substituents independently selected from the group consisting of hydroxyl, methoxycarbonyl, NR^{c}R^{d} (where R^{c} and R^{d} are independently selected from the group consisting of hydrogen, C₁-C₄-alkyl, C₂-C₆-halogenoalkyl or cyclopropyl, or where R^{c} and R^{d} together with the nitrogen atom to which they are bound may form a morpholine ring),
   and
   where in the cycloalkyl ring one CH₂ group may be replaced by CR^{e}R^{f}, O, SO₂ or NR⁵, and where the cycloalkyl may be substituted by 1 substituent selected from the group consisting of methyl, ethyl, n-propyl, isopropyl, hydroxyl, trifluoromethyl, di-(C₁-C₂-alkyl)amino-methyl, cyano, phenyl and pyridinyl, or may be substituted by 1 or 2 fluorine substituents,
      where the phenyl may be substituted by 1 or 2 substituents independently selected from the group consisting of fluorine, chlorine, methoxy and cyano,
      and
      where the pyridinyl may be substituted by 1 methoxy substituent,
      and
      R^{e} and R^{f} together with the carbon atom to which they are bound form another C₃-C₆-cycloalkyl, where again one CH₂ group may be replaced by SO₂,
      and
      R⁵ represents hydrogen, C₁-C₄-alkyl, C₂-C₆-halogenoalkyl substituted by 1 to 3 fluorine atoms, cyclopropyl, methylcarbonyl, methoxycarbonyl or tert-butoxycarbonyl,
   and
   L represents a bond or C₁-C₆-alkanediyl,
      where alkanediyl may be substituted by 1 or 2 substituents independently selected from the group consisting of chlorine, hydroxyl, methoxy, methoxycarbonyl, carboxyl, carbamoyl, cyclopropyl, 1-hydroxycyclopropyl, amino, dimethylamino, (ethyl)(2-hydroxyethyl)amino, tert-butoxycarbonylamino, N₃, azetidin-1-yl (which may be substituted by 1 or 2 fluorine atoms), pyrrolidin-1-yl (which may be substituted by 1 or 2 fluorine atoms), morpholin-4-yl, 1H-1,2,4-triazol-1-yl and N-tert-butoxy-azeditin-3-yl, and additionally by up to 3 fluorine atoms,
   R^{E} represents phenyl, phenoxy, pyridinyl, pyrimidinyl, pyrazinyl, thienyl, pyrazolyl, oxazolyl, isoxazolyl, imidazolyl, 1,2,4-oxadiazolyl, 1,2,4-triazolyl, naphthyl, 1,2,3,4-tetrahydronaphthalen-1-yl, quinolinyl, benzimidazolyl, 2-oxo-2,3-dihydro-1H-benzimidazol-5-yl, indolyl, 2,3-dihydro-1-H-indenyl, benzodioxolyl, 2,3-dihydro-benzodioxinyl, 3,4-dihydro-2H-chromen-4-yl, cyclohexyl, morpholin-4-yl, azetidin-1-yl, pyrrolidin-1-yl, 2-oxo-1,3-oxazolidin-5-yl or 4-cyclopropyl-2,5-dioxoimidazolidin-4-yl,
      where phenyl may be substituted by 1 or 2 substituents independently selected from the group consisting of halogen, C₁-C₄-alkyl, hydroxyl, methoxy, trifluoromethyl, trifluoromethoxy, difluoromethoxy, dimethylaminomethyl, methylsulfonyl, sulfamoyl and pyrrolidin-1-ylmethyl,
      where phenoxy may be substituted by 1 or 2 substituents independently selected from the group consisting of chlorine and methyl,
      where pyridinyl may be substituted by 1 or 2 substituents independently selected from the group consisting of chlorine, methyl, trifluoromethyl, methoxy and 2,2,2-trifluoroethoxy,
      where pyrimidinyl may be substituted by 1 or 2 methyl substituents,
      where pyrazinyl may be substituted by one 2,2,2-trifluoroethoxy substituent,
      where pyrazolyl may be substituted by 1 to 3 substituents independently selected from the group consisting of chlorine, C₁-C₄-alkyl and cyclopropyl,
      where oxazolyl may be substituted by 1 methyl substituent,
      where imidazolyl may be substituted by 1 methyl substituent,
      where 1,2,4-oxadiazol may be substituted by 1 methyl substituent,
      where 1,2,3,4-tetrahydronaphthalen-1-yl may be substituted by 1 methoxy substituent,
      where 1,2,4-triazolyl may be substituted by 1 methyl or ethyl substituent,
      where indolyl may be substituted by 1 or 2 methyl substituents,
      where 2,3-dihydro-1-H-indenyl may be substituted by 1 hydroxyl substituent,
      where 3,4-dihydro-2H-chromen-4-yl may be disubstituted in 2-position by methyl and additionally substituted by 1 substituent selected from methyl and methoxy,
      where azetidin-1-yl may be substituted by 1 fluorine or hydroxyl substituent,
      where pyrrolidin-1-yl may be substituted by 1 fluorine or hydroxyl substituent,
R² represents a group of the formula

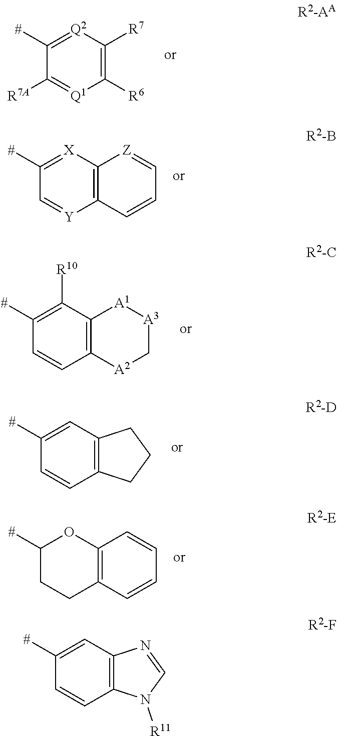

where # is the point of attachment to the pyrazinone ring,
Q¹ represents CR^{8A} or N,
Q² represents CR⁸ or N,
R⁶ represents hydrogen, halogen, C₁-C₄-alkyl, C₁-C₄-halogenoalkyl, C₁-C₄-alkoxy, C₁-C₄-halogenoalkoxy or C₃-C₆-cycloalkyl,
R⁷ represents hydrogen, halogen, C₁-C₄-alkyl, C₁-C₄-halogenoalkyl, C₁-C₄-alkoxy, C₁-C₄-halogenoalkoxy or C₃-C₆-cycloalkyl,
with the proviso, that at least one or R⁶ and R⁷ is not hydrogen,
R^{7A} represents hydrogen or halogen,
R⁸ represents hydrogen or halogen,
R^{8A} represents hydrogen or halogen,
X represents CH or N,
Y represents CH or N,
Z represents CR⁹ or N,
   where at most one of X, Y and Z is N,
   R⁹ represents hydrogen, halogen or C₁-C₄-alkyl,
A¹ represents CH₂, O or NMe,
A² represents CH₂, O or NMe,
A³ represents CH₂ or O,
   where both A¹ and A² are different from O, if A³ is O,
R¹⁰ represents hydrogen or halogen,
R¹¹ represents hydrogen or C₁-C₄-alkyl,
R³ represents hydrogen, halogen, cyano, C₁-C₄-alkyl or C₁-C₂-halogenoalkyl,
   R⁴ represents hydrogen, halogen, C₁-C₄-alkyl, C₁-C₄-halogenoalkyl, C₃-C₆-cycloalkyl, cyano or C₁-C₃-alkoxymethyl,
      where in the cycloalkyl ring one carbon may be replaced by NR¹², and where the cycloalkyl may be substituted by 1 or 2 fluorine atoms,
      R¹² represents hydrogen, C₁-C₄-alkyl or C₁-C₄-alkylaminocarbonyl,
         and the salts thereof, the N-oxides, the solvates thereof and the solvates of the salts or of the N-oxides thereof.

The term “substituted” means that one or more hydrogen atoms on the designated atom or group are replaced with a selection from the indicated group, provided that the designated atom's normal valence under the existing circumstances is not exceeded. Combinations of substituents and/or variables are permissible.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

If within the present text any item is referred to as “as mentioned herein”, it means that it may be mentioned anywhere in the present text.

In the context of the present invention, unless specified otherwise, the substituents are defined as follows:

The term “halogen” or “halogeno” like in combinations e.g. in halogenoalkyl means a fluorine, chlorine, bromine or iodine atom, particularly a fluorine, chlorine or bromine atom, even more particularly fluorine or chlorine.

The term “C₁-C₄-alkyl”, “C₁-C₆-alkyl” and “C₁-C₆-alkyl” means a linear or branched, saturated, monovalent hydrocarbon group having 1, 2, 3, or 4 carbon atoms, 1, 2, 3, 4 or 5 carbon atoms, and 1, 2, 3, 4, 5 or 6 carbon atoms, e.g. a methyl, ethyl, propyl, isopropyl, n-butyl, sec-butyl, iso-butyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tent-butyl group, more particularly 1, 2 or 3 carbon atoms (“C₁-C₃-alkyl”), e.g. a methyl, ethyl, n-propyl or isopropyl group.

The term “C₁-C₆-halogenoalkyl”, “C₂-C₆-halogenoalkyl”, “C₁-C₄-halogenoalkyl”, “C₁-C₃-halogenoalkyl” and “C₁-C₂-halogenoalkyl” represents a linear or branched, saturated, monovalent hydrocarbon group in which the term “alkyl” is as defined supra, and in which one or more of the hydrogen atoms are replaced, identically or differently, with a halogen atom. Particularly, said halogen atom is a fluorine atom. Said C₁-C₆-haloalkyl group is, for example fluoromethyl, difluoromethyl, trifluoromethyl, 2-fluoroethyl, 2,2-difluoroethyl, 2,2,2-trifluoroethyl, pentafluoroethyl, 3,3,3-trifluoropropan-1-yl, 1,1,1-trifluoropropan-2-yl, 1,3-difluoropropan-2-yl, 3-fluoropropan-1-yl, 1,1,1-trifluorobutan-2-yl, and 3,3,3-trifluoro-1-methyl-propan-1-yl.

The term “C₁-C₃-halogenoalkoxy” and “C₁-C₂-halogenoalkoxy” represents a linear or branched, saturated, monovalent C₁-C₃-alkoxy or C₁-C₂-alkoxy group (where alkoxy represents a straight-chain or branched, saturated, monovalent alkoxy radical having 1 to 3 or 1 to 2 carbon atoms, by way of example and with preference methoxy, ethoxy, n-propoxy, isopropoxy), in which one or more of the hydrogen atoms is replaced, identically or differently, with a halogen atom. Particularly, said halogen atom is a fluorine atom. Said C₁-C₃-haloalkoxy group is, for example, fluoromethoxy, difluoromethoxy, trifluoromethoxy, 2,2,2-trifluoroethoxy or pentafluoroethoxy.

The term “C₃-C₆-cycloalkyl” means a saturated, monovalent, monocyclic hydrocarbon ring which contains 3, 4, 5 or 6 carbon atoms. Said C₃-C₆-cycloalkyl group is for example a cyclopropyl, cyclobutyl, cyclopentyl or cyclohexyl group.

The term “C₁-C₅-alkanediyl”, “C₁-C₄-alkanediyl” and “C₂-C₄-alkanediyl” represents a linear or branched, divalent alkyl radical having 1 to 5, 1 to 4 or 2 to 4 carbon atoms, by way of example and with preference methylene (—CH₂—), ethan-1,1-diyl [—CH(CH₃)—], ethan-1,2-diyl [—(CH₂)₂—], propan-1,1-diyl [—CH(CH₂CH₃)—], propan-1,2-diyl [—CH₂CH(CH₃)—], 2-methylpropan-1,1-diyl {—CH[CH(CH₃)₂]—}, 2-methylpropan-1,3-diyl {—CH₂[CH(CH₃)]CH₂—}, butan-1,1-diyl {—CH[(CH₂)₂CH₃]—}, buten-1,2-diyl [—CH₂CH(CH₂CH₃)—], 3-methylbutan-1,1-diyl [—(CH₂)₂CH(CH₃)₂—], 3-methylbutan-1,2-diyl {—CH₂CH[CH(CH₃)₂]—}.

It is possible for the compounds of general formula (I) to exist as isotopic variants. The invention therefore includes one or more isotopic variant(s) of the compounds of general formula (I), particularly deuterium-containing compounds of general formula (I). The term “Isotopic variant” of a compound or a reagent is defined as a compound exhibiting an unnatural proportion of one or more of the isotopes that constitute such a compound. The term “Isotopic variant of the compound of general formula (I)” is defined as a compound of general formula (I) exhibiting an unnatural proportion of one or more of the isotopes that constitute such a compound. The expression “unnatural proportion” means a proportion of such isotope which is higher than its natural abundance. The natural abundances of isotopes to be applied in this context are described in “Isotopic Compositions of the Elements 1997”, Pure Appl. Chem. 1998, 70(1), 217-235, 1998. Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties [such as for example acidity (J. Am. Chem. Soc. 2007, 129, 4490-4497, basicity (J. Am. Chem. Soc. 2005, 127, 9641-9647), lipophilicity (Int. J. Pharm. 1984, 19(3), 271-281)] and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (Toxicol. Appl. Pharmacol. 2000, 169, 102-113). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: Chem. Res. Toxicol. 2013, 26, 410-421; Efavirenz: Toxicol. Appl. Pharmacol. 2000, 169, 102-113). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (J. Med. Chem. 2013, 56, 5208-5212) and Odanacatib (WO2012/112363) are examples for this deuterium effect. Still other cases have been reported in which reduced rates of metabolism result in an increase in exposure of the drug without changing the rate of systemic clearance (e.g. Rofecoxib: ArzneimForschDrugRes 2006, 56, 295-300; Telaprevir: J. Med. Chem. 2009, 52, 7993-8001). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P450.

The compounds of the present invention optionally contain one or more asymmetric centers, depending upon the location and nature of the various substituents desired. It is possible that one or more asymmetric carbon atoms are present in the (R) or (S) configuration, which can result in racemic mixtures in the case of a single asymmetric center, and in diastereomeric mixtures in the case of multiple asymmetric centers. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl. Chem. 1976, 45, 11-30).

In the context of the present invention, the term “enantiomericly pure” is to be understood as meaning that the compound in question with respect to the absolute configuration of the chiral center is present in an enantiomeric excess of more than 95%, preferably more than 97%. The enantiomeric excess, ee, is calculated here by evaluating the corresponding HPLC chromatogram on a chiral phase using the formula below:

$$e e = \frac{{E}^{A} ( area \% ) - {E}^{B} ( area \% )}{{E}^{A} ( area \% ) + {E}^{B} ( area \% )} \times 100 \%$$

(E^{A}: major enantiomer, E^{B}: minor enantiomer)

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it is possible for the compounds of the present invention to exist as tautomers. For example the compounds of formula (I) encompass the tautomer of formula (Ia)

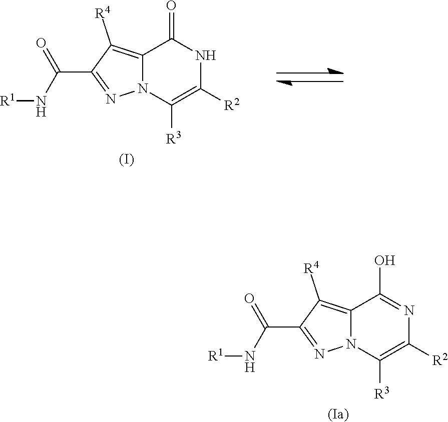

Within this description the compounds according to the invention are drawn in the 4-oxo form.

The present invention includes all possible tautomers of the compounds of the present invention as single tautomers, or as any mixture of said tautomers, in any ratio.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt, in particular as a free acid. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention (see J. Pharm. Sci. 1977, 66, 1-19).

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “×HCl”, “×CF₃COOH”, “×Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Further, the compounds of the present invention can exist as N-oxides, which are defined in that at least one nitrogen of the compounds of the present invention is oxidised in a known manner. The present invention includes all such possible N-oxides.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is used here synonymously with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In one embodiment preference is given to compounds of the formula (I) in which
R¹ represents C₁-C₅-alkyl, C₂-C₄-halogenoalkyl, C₃-C₆-cycloalkyl or the group -L-R^{E},
   where alkyl may be substituted by 1 or 2 substituents independently selected from the group consisting of hydroxyl, cyano, cyclopropyl, 3-fluoroazetidin-1-yl, 3,3-difluoroazetidin-1-yl, NR^{a}R^{b} (where R^{a} and R^{b} are independently selected from the group consisting of hydrogen, methyl, 2,2-difluoroethyl, 2,2,2-trifluoroethyl or cyclopropyl, or where R^{a} and R^{b} together with the nitrogen atom to which they are bound may form a morpholine ring) and C₁-C₂-halogenoalkoxy, where halogenoalkoxy is substituted by 1 to 3 fluorine atoms,
   and
   where halogenoalkyl is substituted by 1 to 6 fluorine atoms and may be further substituted by 1 or 2 hydroxyl substituents,
   and
   where in the cycloalkyl ring one CH₂ group may be replaced by NR⁵, and where the cycloalkyl may be substituted by 1 substituent selected from the group consisting of methyl, trifluoromethyl, diethylamino-methyl, phenyl and pyridin-3-yl,
      where the phenyl may be substituted by 1 or 2 substituents independently selected from the group consisting of fluorine, chlorine and methoxy,
      and
      where the pyridinyl may be substituted by 1 methoxy substituent,
      and
      R⁵ represents hydrogen, methyl, ethyl, n-propyl, 2-methyl-propan-1-yl, 2,2-difluoroethyl, 2,2,2-trifluoroethyl, 1,3-difluoropropan-2-yl or cyclopropyl,
   and
   L represents a bond or C₁-C₅-alkanediyl,
      where alkanediyl may be substituted by 1 or 2 substituents independently selected from the group consisting of hydroxyl, cyclopropyl, 1-hydroxycyclopropyl, amino, dimethylamino, (ethyl)(2-hydroxyethyl)amino, azetidin-1-yl, 3-fluoroazetidin-1-yl, 3-fluoroazetidin-1-yl, pyrrolidin-1-yl, 3-fluoropyrrolidin-1-yl, 3,3-difluoropyrrolidin-1-yl, morpholin-4-yl, and additionally by up to 3 fluorine atoms,
   R^{E} represents phenyl, pyridin-3-yl, pyridin-2-yl, pyrazol-4-yl, pyrazol-1-yl, pyrazol-5-yl or pyrazol-3-yl,
      where phenyl may be substituted by 1 or 2 substituents independently selected from the group consisting of fluorine, chlorine, bromine, methyl, ethyl, hydroxyl, methoxy, tri-fluoromethyl, trifluoromethoxy and difluoromethoxy, where pyridinyl may be substituted by 1 or 2 substituents independently selected from the group consisting of chlorine, methyl, trifluoromethyl, methoxy and 2,2,2-trifluoro-ethoxy,
      where pyrazolyl may be substituted by 1 to 3 substituents independently selected from the group consisting of chlorine, methyl, ethyl, isopropyl and cyclopropyl,
R² represents a group of the formula

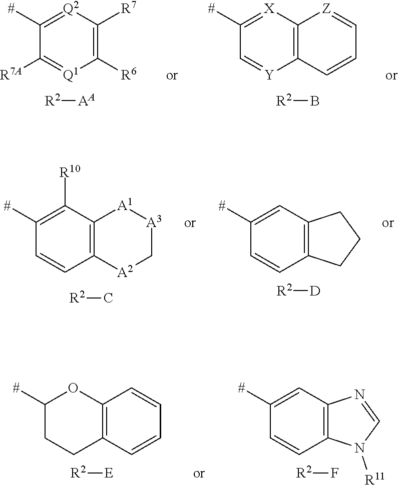

where # is the point of attachment to the pyrazinone ring,
Q¹ represents CR^{8A} or N,
Q² represents CR⁸ or N,
R⁶ represents hydrogen, fluorine, chlorine, methyl, ethyl, difluoromethyl, trifluoromethyl, methoxy, trifluoromethoxy or cyclopropyl,
R⁷ represents hydrogen, fluorine, chlorine, methyl, ethyl, difluoromethyl, trifluoromethyl, methoxy, trifluoromethoxy or cyclopropyl,
with the proviso, that at least one or R⁶ and R⁷ is not hydrogen,
R^{7A} represents hydrogen, fluorine or chlorine.
R⁸ represents hydrogen, fluorine or chlorine,
R^{8A} represents hydrogen, fluorine or chlorine,
X represents CH or N,
Y represents CH or N,
Z represents CR⁹ or N,
where at most one of X, Y and Z is N,
   R⁹ represents hydrogen, fluorine, chlorine, methyl or ethyl,
A¹ represents CH₂, O or NMe,
A² represents CH₂, O or NMe,
A³ represents CH₂ or O,
   where both Al and A² are different from O, if A³ is O,
R¹⁰ represents hydrogen or fluorine,
R¹¹ represents methyl,
R³ represents hydrogen, fluorine, chlorine, cyano, methyl or ethyl,
R⁴ represents hydrogen, fluorine, chlorine, methyl, ethyl, n-propyl, isopropyl, difluoromethyl, trifluoromethyl, pentafluoroethyl, cyclopropyl, 2,2-difluorocyclopropan-1-yl, cyclobutyl, 3,3-difluorocyclobutan-1-yl, piperidin-4-yl, cyano or methoxymethyl,
   where piperidin-4-yl may be substituted in 1-position by methyl, methylaminocarbonyl or ethylaminocarbonyl,
and the salts thereof, the N-oxides, the solvates thereof and the solvates of the salts or of the N-oxides thereof.

Preference is also given to compounds of the formula (I) in which
R¹ represents ethyl, propan-1-yl, propan-2-yl, butan-2-yl, C₂-C₄-halogenoalkyl, cyclopropyl, cyclobutyl or the group -L-R^{E},
   where ethyl, the propanyls and butanyl may be substituted by 1 substituent selected from the group consisting of hydroxyl and cyclopropyl,
   and
   where halogenoalkyl is substituted by 1 to 3 fluorine atoms and may be further substituted by 1 hydroxyl substituent,
   and
   where in the cyclobutyl ring one carbon may be replaced by NR⁵, and where the cyclopropyl or cyclobutyl may be substituted by 1 substituent selected from the group consisting of methyl and phenyl,
      where the phenyl may be substituted by 1 or 2 substituents independently selected from the group consisting of fluorine and methoxy,
      and
      R⁵ represents hydrogen, methyl, ethyl, n-propyl, 2-methyl-propan-1-yl, 2,2-difluoroethyl, 2,2,2-trifluoroethyl, 1,3-difluoropropan-2-yl or cyclopropyl,
   and
   L represents C₁-C₄-alkanediyl,
      where alkanediyl may be substituted by 1 substituent selected from the group consisting of hydroxyl, 1-hydroxycyclopropyl, dimethylamino, and additionally by up to 3 fluorine atoms,
   R^{E} represents phenyl, pyridin-3-yl or pyridin-2-yl,
      where phenyl may be substituted by 1 or 2 substituents independently selected from the group consisting of fluorine, chlorine, methyl, hydroxyl, methoxy, trifluoromethyl, tri-fluoromethoxy and difluoromethoxy,
      where pyridinyl may be substituted by 1 or 2 substituents independently selected from the group consisting of chlorine, trifluoromethyl and 2,2,2-trifluoroethoxy,
R² represents a group of the formula

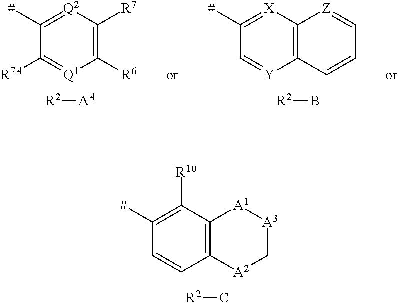

where # is the point of attachment to the pyrazinone ring,
Q¹ represents CR^{8A},
Q² represents CR⁸,
R⁶ represents hydrogen, fluorine, chlorine, methyl, difluoromethyl, trifluoromethyl methoxy or trifluoromethoxy,
R⁷ represents hydrogen, fluorine, chlorine, methyl, difluoromethyl, trifluoromethyl, methoxy or trifluoromethoxy,
with the proviso, that at least one or R⁶ and R⁷ is not hydrogen,
R^{7A} represents hydrogen, fluorine or chlorine.
R⁸ represents hydrogen or fluorine,
R^{8A} represents hydrogen or fluorine,
X represents CH or N,
Y represents CH or N,
Z represents CR⁹,
where at most one of X and Y is N,
   R⁹ represents hydrogen or methyl,
A¹ and A² represent at the same time CH₂ or O, or
one of A¹ and A² represents O and the other represents NMe,
A³ represents CH₂,
R¹⁰ represents hydrogen,
R³ represents hydrogen, fluorine, chlorine or methyl,
R⁴ represents hydrogen, chlorine, methyl, isopropyl, trifluoromethyl, pentafluoroethyl, cyclopropyl, 2,2-difluorocyclopropan-1-yl, 3,3-difluorocyclobutan-1-yl, cyano or methoxymethyl,
   and the salts thereof, the N-oxides, the solvates thereof and the solvates of the salts or of the N-oxides thereof.

Preference is also given to compounds of the formula (I) in which
R¹ represents ethyl, propan-1-yl, propan-2-yl, 3,3,3-trifluoropropan-1-yl, 3,3-difluoropropan-1-yl, 1,1,1-trifluoropropan-2-yl, 4,4,4-trifluorobutan-1-yl, cyclobutyl or the group -L-R^{E},
   where ethyl may be substituted by 1 cyclopropyl substituent,
   and
   where the propanyls may be substituted by 1 hydroxyl substituent,
   and
   where the di- and trifluoropropanyls and the trifluorobutanyl may be further substituted by 1 hydroxyl substituent,
   and
   where in the cyclobutyl ring one carbon is replaced by NR⁵, and where the cyclobutyl may be substituted by 1 substituent selected from the group consisting of methyl and phenyl,
      where the phenyl may be substituted by 1 substituent selected from the group consisting of fluorine and methoxy,
      and
      R⁵ represents hydrogen or methyl,
   and
   L represents C₁-C₄-alkanediyl,
      where alkanediyl may be substituted by 1 substituent selected from the group consisting of hydroxyl and 1-hydroxycyclopropyl, and additionally by up to 3 fluorine atoms,
   R^{E} represents phenyl,
      where phenyl may be substituted by 1 or 2 substituents selected from the group consisting of fluorine, chlorine, hydroxyl, methoxy, trifluoromethyl, trifluoromethoxy and difluoromethoxy,
R² represents 4-chlorophenyl, 3-chlorophenyl, 4-chloro-3-fluorophenyl, 3-chloro-4-fluorophenyl, 4-chloro-3-methylphenyl, 3-chloro-4-methylphenyl, 4-chloro-3-difluoromethyl-phenyl, 3-chloro-4-difluoromethyl-phenyl, 4-fluoro-3-methoxyphenyl, 3-fluoro-4-methoxyphenyl, 4-chloro-3-methoxyphenyl, 3-chloro-4-methoxyphenyl, 4-chloro-3-trifluoromethoxy-phenyl, 3-chloro-4-trifluoromethoxy-phenyl, 3,4-dichlorophenyl, 3,4-dimethylphenyl, 3-fluoro-4-methylphenyl, 4-fluoro-3-methylphenyl, 4-methyl-3-trifluoromethylphenyl, 3-methyl-4-trifluoromethylphenyl, 2,3-difluoro-4-methylphenyl, 2-fluoro-3,4-dimethylphenyl, 4-chloro-3,5-difluoropheynl, 4-chloro-2-fluoro-5-methyl, 4,5-dichloro-2-fluorophenyl, 4-chloro-2,5-difluorophenyl, or represents a group of the formula

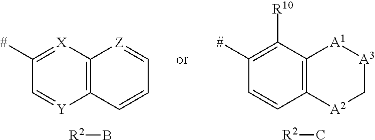

where # is the point of attachment to the pyrazinone ring,
X, Y and Z all represent CH or
X is CH, Y is N, Z is CR⁹ and R⁹ is methyl,
A¹ is O, A² is O, A³ is CH₂ and R¹⁹ is hydrogen,
R³ represents hydrogen, fluorine or methyl,
R⁴ represents hydrogen, methyl, isopropyl, trifluoromethyl or cyclopropyl, and the salts thereof, the N-oxides, the solvates thereof and the solvates of the salts or of the N-oxides thereof.

Preference is also given to compounds of the formula (I) in which
R¹ represents 1-cyclopropylethyl, 1-hydroxypropan-2-yl, 3,3,3-trifluoro-2-hydroxpropan-1-yl, 3,3-difluoro-2-hydroxpropan-1-yl, 4,4,4-trifluoro-3-hydroxy-butan-1-yl, 3-(4-fluorophenyl)-1-methylazetidin-3-yl, 3-(4-methoxyphenyl)-1-methylazetidin-3-yl or the group -L-R^{E},
   where
   L represents an C₁-C₄-alkanediyl selected from the group consisting of 1-hydroxycyclopropyl-methan-1,1,-diyl [—CH(1-hydroxycyclopropyl)-], ethan-1,1-diyl [—CH(CH₃)—], 2-hydroxy-ethan-1,1-diyl [—CH(CH₂OH)—], 3-hydroxy-propan-1,1-diyl {—CH[(CH₂)₂OH]—}, 2-hydroxy-2-methylpropan-1,1-diyl {—CH[C(CH₃)₂OH]—} and 2,2-difluoro-3-hydroxy-propan-1,1-diyl [—CH(CF₂CH₂OH)—],
   R^{E} represents phenyl,
      where phenyl may be substituted in 4-position by fluorine, hydroxyl, methoxy or trifluoromethyl, or where phenyl may be disubstituted in 3- and 4-position by fluorine,
R² represents 4-chlorophenyl, 3-chlorophenyl, 4-chloro-3-fluorophenyl, 3-chloro-4-fluorophenyl, 4-chloro-3-methylphenyl, 3-chloro-4-methylphenyl, 3,4-dichlorophenyl, 3,4-dimethylphenyl, 3-fluoro-4-methylphenyl, 4-methyl-3-trifluoromethylphenyl, 3-methyl-4-trifluoromethylphenyl or 4-chloro-2,5-difluorophenyl,
R³ represents hydrogen,
R⁴ represents isopropyl, trifluoromethyl or cyclopropyl,
   and the salts thereof, the N-oxides, the solvates thereof and the solvates of the salts or of the N-oxides thereof.

In another embodiment the invention provides compounds of the formula (I) in which
R¹ represents C₁-C₆-alkyl, C₂-C₆-halogenoalkyl, C₃-C₆-cycloalkyl or the group -L-R^{E},
   where alkyl may be substituted by 1 or 2 substituents independently selected from the group consisting of hydroxyl, cyano, cyclopropyl, methoxy, methylsulfonyl, carbamoyl, NR^{a}R^{b} (where R^{a} and R^{b} are independently selected from the group consisting of hydrogen, C₁-C₄-alkyl, C₂-C₆-halogenoalkyl or cyclopropyl, or where R^{a} and R^{b} together with the nitrogen atom to which they are bound may form a morpholine ring) and C₁-C₃-halogenoalkoxy, where halogenoalkoxy is substituted by 1 to 3 fluorine atoms,
   and
   where halogenoalkyl is substituted by 1 to 6 fluorine atoms and may be further substituted by 1 or 2 substituents independently selected from the group consisting of hydroxyl, methoxycarbonyl, NR^{c}R^{d} (where R^{c} and R^{d} are independently selected from the group consisting of hydrogen, C₁-C₄-alkyl, C₂-C₆-halogenoalkyl or cyclopropyl, or where R^{c} and R^{d} together with the nitrogen atom to which they are bound may form a morpholine ring),
   and
   where in the cycloalkyl ring one CH₂ group may be replaced by CR^{e}R^{f}, O, SO₂ or NR⁵, and where the cycloalkyl may be substituted by 1 substituent selected from the group consisting of methyl, ethyl, hydroxyl, trifluoromethyl, di-(C₁-C₂-alkyl)amino-methyl, cyano, phenyl and pyridinyl,
      where the phenyl may be substituted by 1 or 2 substituents independently selected from the group consisting of fluorine, chlorine, methoxy and cyano,
      and
      where the pyridinyl may be substituted by 1 methoxy substituent,
      and
      R^{e} and R^{f} together with the carbon atom to which they are bound form another C₃-C₆-cycloalkyl, where again one CH₂ group may be replaced by SO₂,
      and
      R⁵ represents hydrogen, C₁-C₄-alkyl, C₂-C₆-halogenoalkyl substituted by 1 to 3 fluorine atoms, cyclopropyl, methylcarbonyl, methoxycarbonyl or tert-butoxycarbonyl,
   and
   L represents a bond or C₁-C₆-alkanediyl,
      where alkanediyl may be substituted by 1 or 2 substituents independently selected from the group consisting of chlorine, hydroxyl, methoxy, methoxycarbonyl, carboxyl, carbamoyl, cyclopropyl, 1-hydroxycyclopropyl, amino, dimethylamino, (ethyl)(2-hydroxyethyl)amino, tert-butoxycarbonylamino, N3, 3-fluoroazetidin-1-yl, pyrrolidin-1-yl, morpholin-4-yl, 1H-1,2,4-triazol-1-yl and N-tert-butoxy-azeditin-3-yl, and additionally by up to 3 fluorine atoms,
   R^{E} represents phenyl, phenoxy, pyridinyl, pyrimidinyl, pyrazinyl, thienyl, pyrazolyl, oxazolyl, imidazolyl, 1,2,4-oxadiazolyl, 1,2,4-triazolyl, naphthyl, 1,2,3,4-tetrahydronaphthalen-1-yl, quinolinyl, benzimidazolyl, 2-oxo-2,3-dihydro-1H-benzimidazol-5-yl, indolyl, 2,3-di-hydro-1-H-indenyl, benzodioxolyl, 2,3-dihydro-benzodioxinyl, 3,4-dihydro-2H-chromen-4-yl, cyclohexyl, morpholin-4-yl, azetidin-1-yl, pyrrolidin-1-yl, 2-oxo-1,3-oxazolidin-5-yl or 4-cyclopropyl-2,5-dioxoimidazolidin-4-yl,
      where phenyl may be substituted by 1 or 2 substituents independently selected from the group consisting of halogen, C₁-C₄-alkyl, hydroxyl, methoxy, trifluoromethyl, trifluoromethoxy, difluoromethoxy, dimethylaminomethyl, methylsulfonyl, sulfamoyl and pyrrolidin-1-ylmethyl,
      where phenoxy may be substituted by 1 or 2 substituents independently selected from the group consisting of chlorine and methyl,
      where pyridinyl may be substituted by 1 or 2 substituents independently selected from the group consisting of chlorine, methyl, trifluoromethyl, methoxy and 2,2,2-trifluoroethoxy,
      where pyrimidinyl may be substituted by 1 or 2 methyl substituents,
      where pyrazinyl may be substituted by one 2,2,2-trifluoroethoxy substituent,
      where pyrazolyl may be substituted by 1 to 3 substituents independently selected from the group consisting of chlorine, C₁-C₄-alkyl and cyclopropyl,
      where oxazolyl may be substituted by 1 methyl substituent,
      where imidazolyl may be substituted by 1 methyl substituent,
      where 1,2,4-oxadiazol may be substituted by 1 methyl substituent,
      where 1,2,3,4-tetrahydronaphthalen-1-yl may be substituted by 1 methoxy substituent,
      where 1,2,4-triazolyl may be substituted by 1 methyl or ethyl substituent,
      where indolyl may be substituted by 1 or 2 methyl substituents,
      where 2,3-dihydro-1-H-indenyl may be substituted by 1 hydroxyl substituent,
      where 3,4-dihydro-2H-chromen-4-yl may be disubstituted in 2-position by methyl and additionally substituted by 1 substituent selected from methyl and methoxy,
      where azetidin-1-yl may be substituted by 1 fluorine or hydroxyl substituent,
      where pyrrolidin-1-yl may be substituted by 1 fluorine or hydroxyl substituent,
R² represents a group of the formula

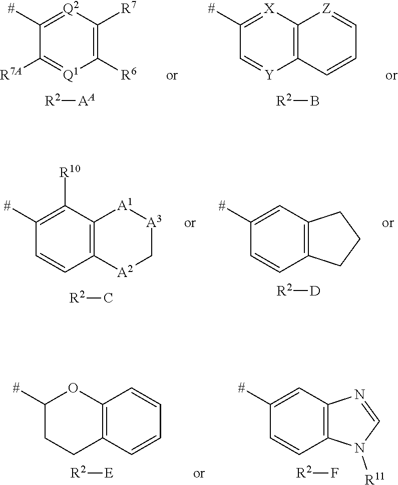

where # is the point of attachment to the pyrazinone ring,
Q¹ represents CH or N,
Q² represents CR⁸ or N,
R⁶ represents halogen, C₁-C₄-alkyl, C₁-C₄-halogenoalkyl or C₃-C₆-cycloalkyl,
R⁷ represents halogen, C₁-C₄-alkyl, C₁-C₄-halogenoalkyl or C₃-C₆-cycloalkyl,
R⁸ represents hydrogen or halogen,
X represents CH or N,
Y represents CH or N,
Z represents CR⁹ or N,
   where at most one of X, Y and Z is N,
   R⁹ represents hydrogen, halogen or C₁-C₄-alkyl,
A² represents CH₂, O or NMe,
A² represents CH₂, O or NMe,
A³ represents CH₂ or O,
   where both A¹ and A² are different from O, if A³ is O,
R¹⁰ represents hydrogen or halogen,
R¹¹ represents hydrogen or C₁-C₄-alkyl,
R³ represents hydrogen, halogen, cyano, C₁-C₄-alkyl or C₁-C₂-halogenoalkyl,
R⁴ represents hydrogen, halogen, C₁-C₄-alkyl, C₁-C₄-halogenoalkyl, C₃-C₆-cycloalkyl, cyano or C₁-C₃-alkoxymethyl,
   where in the cycloalkyl ring one carbon may be replaced by NR¹²,
   R¹² represents hydrogen, C₁-C₄-alkyl or C₁-C₄-alkylaminocarbonyl,
      and the salts thereof, the N-oxides, the solvates thereof and the solvates of the salts or of the N-oxides thereof.

In another embodiment preference is given to compounds of the formula (I) in which
R¹ represents C₁-C₅-alkyl, C₂-C₄-halogenoalkyl, C₃-C₆-cycloalkyl or the group -L-R^{E},
   where alkyl may be substituted by 1 or 2 substituents independently selected from the group consisting of hydroxyl, cyano, cyclopropyl, NR^{a}R^{b} (where R^{a} and R^{b} are independently selected from the group consisting of hydrogen, methyl, 2,2-difluoroethyl, 2,2,2-trifluoroethyl or cyclopropyl, or where R^{a} and Rb together with the nitrogen atom to which they are bound may form a morpholine ring) and C₁-C₂-halogenoalkoxy, where halogenoalkoxy is substituted by 1 to 3 fluorine atoms,
   and
   where halogenoalkyl is substituted by 1 to 6 fluorine atoms and may be further substituted by 1 or 2 hydroxyl substituents,
   and
   where in the cycloalkyl ring one CH₂ group may be replaced by NR⁵, and where the cycloalkyl may be substituted by 1 substituent selected from the group consisting of methyl, trifluoromethyl, diethylamino-methyl, phenyl and pyridin-3-yl,
      where the phenyl may be substituted by 1 or 2 substituents independently selected from the group consisting of fluorine, chlorine and methoxy,
      and
      where the pyridinyl may be substituted by 1 methoxy substituent,
      and
      R⁵ represents hydrogen, C₁-C₂-alkyl, 2,2-difluoroethyl, 2,2,2-trifluoroethyl or cyclopropyl,
   and
   L represents a bond or C₁-C₆-alkanediyl,
      where alkanediyl may be substituted by 1 or 2 substituents independently selected from the group consisting of hydroxyl, amino, dimethylamino, (ethyl)(2-hydroxyethyl)amino, morpholin-4-yl, and additionally by up to 3 fluorine atoms,
   R^{E} represents phenyl, pyridin-3-yl, pyridin-2-yl, pyrazol-4-yl, pyrazol-1-yl, pyrazol-5-yl or pyrazol-3-yl,
      where phenyl may be substituted by 1 or 2 substituents independently selected from the group consisting of fluorine, chlorine, bromine, methyl, ethyl, hydroxyl, methoxy, trifluoromethyl, trifluoromethoxy and difluoromethoxy,
      where pyridinyl may be substituted by 1 or 2 substituents independently selected from the group consisting of chlorine, methyl, trifluoromethyl, methoxy and 2,2,2-trifluoroethoxy,
      where pyrazolyl may be substituted by 1 to 3 substituents independently selected from the group consisting of chlorine, methyl, ethyl, isopropyl and cyclopropyl,
R² represents a group of the formula

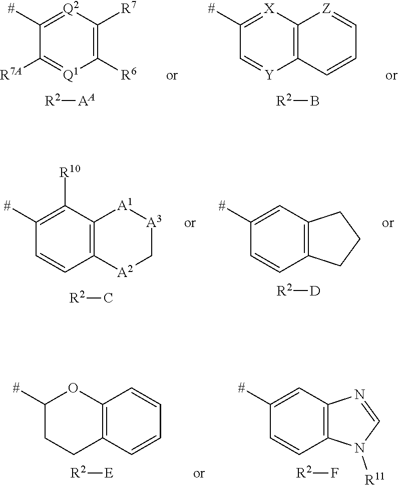

where # is the point of attachment to the pyrazinone ring,
Q¹ represents CH or N,
Q² represents CR⁸ or N,
R⁶ represents fluorine, chlorine, methyl, ethyl, trifluoromethyl or cyclopropyl,
R⁷ represents fluorine, chlorine, methyl, ethyl, trifluoromethyl or cyclopropyl,
R⁸ represents hydrogen, fluorine or chlorine,
X represents CH or N,
Y represents CH or N,
Z represents CR⁹ or N,
   where at most one of X, Y and Z is N,
   R⁹ represents hydrogen, fluorine, chlorine, methyl or ethyl,
A¹ represents CH₂, O or NMe,
A² represents CH₂, O or NMe,
A³ represents CH₂ or O,
   where both A¹ and A² are different from O, if A³ is O,
R¹⁰ represents hydrogen or fluorine,
R¹¹ represents methyl,
R³ represents hydrogen, fluorine, chlorine, cyano, methyl or ethyl,
R⁴ represents hydrogen, fluorine, chlorine, methyl, ethyl, n-propyl, isopropyl, difluoromethyl, trifluoromethyl, cyclopropyl, piperidin-4-yl, cyano or methoxymethyl,
   where piperidin-4-yl may be substituted in 1-position by methyl, methylaminocarbonyl or ethylaminocarbonyl,
      and the salts thereof, the N-oxides, the solvates thereof and the solvates of the salts or of the N-oxides thereof.

In another embodiment preference is also given to compounds of the formula (I) in which
R¹ represents ethyl, propan-1-yl, propan-2-yl, butan-2-yl, C₁-C₃-halogenoalkyl, cyclopropyl, cyclobutyl or the group -L-R^{E},
   where ethyl, the propanyls and butanyl may be substituted by 1 substituent selected from the group consisting of hydroxyl and cyclopropyl,
   and
   where halogenoalkyl is substituted by 1 to 3 fluorine atoms and may be further substituted by 1 hydroxyl substituent,
   and
   where in the cyclobutyl ring one carbon may be replaced by NR⁵, and where the cyclopropyl or cyclobutyl may be substituted by 1 substituent selected from the group consisting of methyl and phenyl,
      where the phenyl may be substituted by 1 or 2 substituents independently selected from the group consisting of fluorine and methoxy,
      and
      R⁵ represents hydrogen, methyl or ethyl,
   and
   L represents C₂-C₄-alkanediyl,
      where alkanediyl may be substituted by 1 substituent selected from the group consisting of hydroxyl, dimethylamino, and additionally by up to 3 fluorine atoms,
   R^{E} represents phenyl, pyridin-3-yl or pyridin-2-yl,
      where phenyl may be substituted by 1 or 2 substituents independently selected from the group consisting of fluorine, chlorine, methyl, hydroxyl, methoxy, trifluoromethyl, trifluoromethoxy and difluoromethoxy,
      where pyridinyl may be substituted by 1 or 2 substituents independently selected from the group consisting of chlorine, trifluoromethyl and 2,2,2-trifluoroethoxy,
R² represents a group of the formula

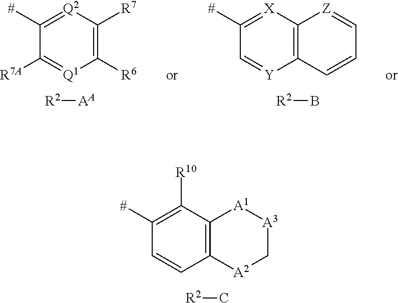

where # is the point of attachment to the pyrazinone ring,
Q¹ represents CH,
Q² represents CR⁸,
R⁶ represents fluorine, chlorine, methyl or trifluoromethyl,
R⁷ represents fluorine, chlorine, methyl or trifluoromethyl,
R⁸ represents hydrogen or fluorine,
X represents CH or N,
Y represents CH or N,
Z represents CR⁹,
   where at most one of X and Y is N,
   R⁹ represents hydrogen or methyl,
A¹ and A² represent at the same time CH₂ or O, or
one of A¹ and A² represents O and the other represents NMe,
A³ represents CH₂,
R¹⁰ represents hydrogen,
R³ represents hydrogen, fluorine, chlorine or methyl,
R⁴ represents hydrogen, chlorine, methyl, isopropyl, trifluoromethyl, cyclopropyl, cyano or methoxymethyl,
   and the salts thereof, the N-oxides, the solvates thereof and the solvates of the salts or of the N-oxides thereof.

In another embodiment preference is also given to compounds of the formula (I) in which
R¹ represents ethyl, propan-1-yl, propan-2-yl, 3,3,3-trifluoropropan-1-yl, 1,1,1-trifluoropropan-2-yl, cyclobutyl or the group -L-R^{E},
   where ethyl may be substituted by 1 cyclopropyl substituent,
   and
   where the propanyls may be substituted by 1 hydroxyl substituent,
   and
   where the trifluoropropanyls may be further substituted by 1 hydroxyl substituent,
   and
   where in the cyclobutyl ring one carbon is replaced by NR⁵, and where the cyclobutyl may be substituted by 1 substituent selected from the group consisting of methyl and phenyl,
      where the phenyl may be substituted by 1 substituent selected from the group consisting of fluorine and methoxy,
      and
      R⁵ represents hydrogen or methyl,
   and
   L represents C₂-C₄-alkanediyl,
      where alkanediyl may be substituted by 1 hydroxyl substituent, and additionally by up to 3 fluorine atoms,
   R^{E} represents phenyl,
      where phenyl may be substituted by 1 substituent selected from the group consisting of fluorine, chlorine, hydroxyl, methoxy, trifluoromethyl, trifluoromethoxy and difluoromethoxy,
R² represents 4-chloro-3-methylphenyl, 3-chloro-4-methylphenyl, 3,4-dimethylphenyl, 3-fluoro-4-methylphenyl, 4-fluoro-3-methylphenyl, 4-methyl-3-trifluoromethylphenyl, 3-methyl-4-trifluoromethylphenyl, 2,3-difluoro-4-methylphenyl, 2-fluoro-3,4-dimethylphenyl, or represents a group of the formula

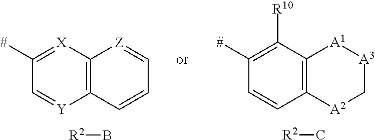

where # is the point of attachment to the pyrazinone ring,
X, Y and Z all represent CH or
X is CH, Y is N, Z is CR⁹ and R⁹ is methyl,
A¹ is O, A² is O, A³ is CH₂ and R¹⁹ is hydrogen,
R³ represents hydrogen, fluorine or methyl,
R⁴ represents hydrogen, methyl, trifluoromethyl or cyclopropyl,
   and the salts thereof, the N-oxides, the solvates thereof and the solvates of the salts or of the N-oxides thereof.

In another embodiment preference is also given to compounds of the formula (I) in which
R¹ represents 1-cyclopropylethyl, 1-hydroxypropan-2-yl, 3,3,3-trifluoro-2-hydroxpropan-1-yl, 3-(4-fluorophenyl)-1-methylazetidin-3-yl, 3-(4-methoxyphenyl)-1-methylazetidin-3-yl or the group -L-R^{E},
   where
   L represents an C₂-C₄-alkanediyl selected from the group consisting of ethan-1,1-diyl [—CH(CH₃)—], 2-hydroxy-ethan-1,1-diyl [—CH(CH₂OH)—], 3-hydroxy-propan-1,1-diyl {—CH[(CH₂)₂OH]—}, 2-hydroxy-2-methylpropan-1,1-diyl {—CH[C(CH₃)₂OH]—} and 2,2-difluoro-3-hydroxy-propan-1,1-diyl [—CH(CF₂CH₂OH)—],
   R^{E} represents phenyl,
      where phenyl may be substituted in 4-position by fluorine, hydroxyl, methoxy or trifluoromethyl,
R² represents 4-chloro-3-methylphenyl, 3-chloro-4-methylphenyl, 3,4-dimethylphenyl, 3-fluoro-4-methylphenyl, 4-methyl-3-trifluoromethylphenyl or 3-methyl-4-trifluoromethylphenyl,
R³ represents hydrogen,
R⁴ represents trifluoromethyl or cyclopropyl,
   and the salts thereof, the N-oxides, the solvates thereof and the solvates of the salts or of the N-oxides thereof.

Preference is also given to compounds of the formula (I) in which
R¹ represents C₁-C₅-alkyl, C₂-C₄-halogenoalkyl, C₃-C₆-cycloalkyl or the group -L-R^{E},
   where alkyl may be substituted by 1 or 2 substituents independently selected from the group consisting of hydroxyl, cyano, cyclopropyl, 3-fluoroazetidin-1-yl, 3,3-difluoroazetidin-1-yl, NR^{a}R^{b} (where R^{a} and R^{b} are independently selected from the group consisting of hydrogen, methyl, 2,2-difluoroethyl, 2,2,2-trifluoroethyl or cyclopropyl, or where R^{a} and R^{b} together with the nitrogen atom to which they are bound may form a morpholine ring) and C₁-C₂-halogenoalkoxy, where halogenoalkoxy is substituted by 1 to 3 fluorine atoms,
   and
   where halogenoalkyl is substituted by 1 to 6 fluorine atoms and may be further substituted by 1 or 2 hydroxyl substituents,
   and
   where in the cycloalkyl ring one CH₂ group may be replaced by NR⁵, and where the cycloalkyl may be substituted by 1 substituent selected from the group consisting of methyl, trifluoromethyl, diethylamino-methyl, phenyl and pyridin-3-yl,
      where the phenyl may be substituted by 1 or 2 substituents independently selected from the group consisting of fluorine, chlorine and methoxy,
      and
      where the pyridinyl may be substituted by 1 methoxy substituent,
      and
      R⁵ represents hydrogen, methyl, ethyl, n-propyl, 2-methyl-propan-1-yl, 2,2-difluoroethyl, 2,2,2-trifluoroethyl, 1,3-difluoropropan-2-yl or cyclopropyl,
   and
   L represents a bond or C₁-C₅-alkanediyl,
      where alkanediyl may be substituted by 1 or 2 substituents independently selected from the group consisting of hydroxyl, cyclopropyl, 1-hydroxycyclopropyl, amino, dimethylamino, (ethyl)(2-hydroxyethyl)amino, azetidin-1-yl, 3-fluoroazetidin-1-yl, 3-fluoroazetidin-1-yl, pyrrolidin-1-yl, 3-fluoropyrrolidin-1-yl, 3,3-difluoropyrrolidin-1-yl morpholin-4-yl, and additionally by up to 3 fluorine atoms,
   R^{E} represents phenyl, pyridin-3-yl, pyridin-2-yl, pyrazol-4-yl, pyrazol-1-yl, pyrazol-5-yl or pyrazol-3-yl,
      where phenyl may be substituted by 1 or 2 substituents independently selected from the group consisting of fluorine, chlorine, bromine, methyl, ethyl, hydroxyl, methoxy, trifluoromethyl, trifluoromethoxy and difluoromethoxy,
      where pyridinyl may be substituted by 1 or 2 substituents independently selected from the group consisting of chlorine, methyl, trifluoromethyl, methoxy and 2,2,2-trifluoroethoxy,
      where pyrazolyl may be substituted by 1 to 3 substituents independently selected from the group consisting of chlorine, methyl, ethyl, isopropyl and cyclopropyl.

Preference is also given to compounds of the formula (I) in which
R¹ represents C₁-C₅-alkyl, C₂-C₄-halogenoalkyl, C₃-C₄-cycloalkyl or the group -L-R^{E},
   where alkyl may be substituted by 1 or 2 substituents independently selected from the group consisting of hydroxyl, cyano, cyclopropyl, NR^{a}R^{b} (where R^{a} and R^{b} are independently selected from the group consisting of hydrogen, methyl, 2,2-difluoroethyl, 2,2,2-trifluoroethyl or cyclopropyl, or where R^{a} and R^{b} together with the nitrogen atom to which they are bound may form a morpholine ring) and C₁-C₂-halogenoalkoxy, where halogenoalkoxy is substituted by 1 to 3 fluorine atoms,
   and
   where halogenoalkyl is substituted by 1 to 6 fluorine atoms and may be further substituted by 1 or 2 hydroxyl substituents,
   and
   where in the cycloalkyl ring one CH₂ group may be replaced by NR⁵, and where the cycloalkyl may be substituted by 1 substituent selected from the group consisting of methyl, trifluoromethyl, diethylamino-methyl, phenyl and pyridin-3-yl,
      where the phenyl may be substituted by 1 or 2 substituents independently selected from the group consisting of fluorine, chlorine and methoxy,
      and
      where the pyridinyl may be substituted by 1 methoxy substituent,
      and
      R⁵ represents hydrogen, C₁-C₂-alkyl, 2,2-difluoroethyl, 2,2,2-trifluoroethyl or cyclopropyl,
   and
   L represents a bond or C₁-C₅-alkanediyl,
      where alkanediyl may be substituted by 1 or 2 substituents independently selected from the group consisting of hydroxyl, amino, dimethylamino, (ethyl)(2-hydroxyethyl)amino, morpholin-4-yl, and additionally by up to 3 fluorine atoms,
   R^{E} represents phenyl, pyridin-3-yl, pyridin-2-yl, pyrazol-4-yl, pyrazol-1-yl, pyrazol-5-yl or pyrazol-3-yl,
      where phenyl may be substituted by 1 or 2 substituents independently selected from the group consisting of fluorine, chlorine, bromine, methyl, ethyl, hydroxyl, methoxy, trifluoromethyl, trifluoromethoxy and difluoromethoxy,
      where pyridinyl may be substituted by 1 or 2 substituents independently selected from the group consisting of chlorine, methyl, trifluoromethyl, methoxy and 2,2,2-trifluoroethoxy,
      where pyrazolyl may be substituted by 1 to 3 substituents independently selected from the group consisting of chlorine, methyl, ethyl, isopropyl and cyclopropyl.

Preference is also given to compounds of the formula (I) in which
R¹ represents ethyl, propan-1-yl, propan-2-yl, butan-2-yl, C₂-C₄-halogenoalkyl, cyclopropyl, cyclobutyl or the group -L-R^{E},
   where ethyl, the propanyls and butanyl may be substituted by 1 substituent selected from the group consisting of hydroxyl and cyclopropyl,
   and
   where halogenoalkyl is substituted by 1 to 3 fluorine atoms and may be further substituted by 1 hydroxyl substituent,
   and
   where in the cyclobutyl ring one carbon may be replaced by NR⁵, and where the cyclopropyl or cyclobutyl may be substituted by 1 substituent selected from the group consisting of methyl and phenyl,
      where the phenyl may be substituted by 1 or 2 substituents independently selected from the group consisting of fluorine and methoxy,
      and
      R⁵ represents hydrogen, methyl, ethyl, n-propyl, 2-methyl-propan-1-yl, 2,2-difluoroethyl, 2,2,2-trifluoroethyl, 1,3-difluoropropan-2-yl or cyclopropyl,
   and
   L represents C₁-C₄-alkanediyl,
      where alkanediyl may be substituted by 1 substituent selected from the group consisting of hydroxyl, 1-hydroxycyclopropyl, dimethylamino, and additionally by up to 3 fluorine atoms,
   R^{E} represents phenyl, pyridin-3-yl or pyridin-2-yl,
      where phenyl may be substituted by 1 or 2 substituents independently selected from the group consisting of fluorine, chlorine, methyl, hydroxyl, methoxy, trifluoromethyl, trifluoromethoxy and difluoromethoxy,
      where pyridinyl may be substituted by 1 or 2 substituents independently selected from the group consisting of chlorine, trifluoromethyl and 2,2,2-trifluoroethoxy.

Preference is also given to compounds of the formula (I) in which
R¹ represents ethyl, propan-1-yl, propan-2-yl, butan-2-yl, C₁-C₃-halogenoalkyl, cyclopropyl, cyclobutyl or the group -L-R^{E},
   where ethyl, the propanyls and butanyl may be substituted by 1 substituent selected from the group consisting of hydroxyl and cyclopropyl,
   and
   where halogenoalkyl is substituted by 1 to 3 fluorine atoms and may be further substituted by 1 hydroxyl substituent,
   and
   where in the cyclobutyl ring one carbon may be replaced by NR⁵, and where the cyclopropyl or cyclobutyl may be substituted by 1 substituent selected from the group consisting of methyl and phenyl,
      where the phenyl may be substituted by 1 or 2 substituents independently selected from the group consisting of fluorine and methoxy,
      and
      R⁵ represents hydrogen, methyl or ethyl,
   and
   L represents C₂-C₄-alkanediyl,
      where alkanediyl may be substituted by 1 substituent selected from the group consisting of hydroxyl, dimethylamino, and additionally by up to 3 fluorine atoms,
   R^{E} represents phenyl, pyridin-3-yl or pyridin-2-yl,
      where phenyl may be substituted by 1 or 2 substituents independently selected from the group consisting of fluorine, chlorine, methyl, hydroxyl, methoxy, trifluoromethyl, trifluoromethoxy and difluoromethoxy,
      where pyridinyl may be substituted by 1 or 2 substituents independently selected from the group consisting of chlorine, trifluoromethyl and 2,2,2-trifluoroethoxy.

Preference is also given to compounds of the formula (I) in which
R¹ represents ethyl, propan-1-yl, propan-2-yl, 3,3,3-trifluoropropan-1-yl, 3,3-difluoropropan-1-yl, 1,1,1-trifluoropropan-2-yl, 4,4,4-trifluorobutan-1-yl, cyclobutyl or the group -L-R^{E},
   where ethyl may be substituted by 1 cyclopropyl substituent,
   and
   where the propanyls may be substituted by 1 hydroxyl substituent,
   and
   where the di- and trifluoropropanyls and the trifluorobutanyl may be further substituted by 1 hydroxyl substituent,
   and
   where in the cyclobutyl ring one carbon is replaced by NR⁵, and where the cyclobutyl may be substituted by 1 substituent selected from the group consisting of methyl and phenyl,
      where the phenyl may be substituted by 1 substituent selected from the group consisting of fluorine and methoxy,
      and
      R⁵ represents hydrogen or methyl,
   and
   L represents C₁-C₄-alkanediyl,
      where alkanediyl may be substituted by 1 substituent selected from the group consisting of hydroxyl and 1-hydroxycyclopropyl, and additionally by up to 3 fluorine atoms,
   R^{E} represents phenyl,
      where phenyl may be substituted by 1 or 2 substituents selected from the group consisting of fluorine, chlorine, hydroxyl, methoxy, trifluoromethyl, trifluoromethoxy and difluoromethoxy.

Preference is also given to compounds of the formula (I) in which
R¹ represents ethyl, propan-1-yl, propan-2-yl, 3,3,3-trifluoropropan-1-yl, 1,1,1-trifluoropropan-2-yl, cyclobutyl or the group -L-R^{E},
   where ethyl may be substituted by 1 cyclopropyl substiuent,
   and
   where the propanyls may be substituted by 1 hydroxyl substituent,
   and
   where the trifluoropropanyls may be further substituted by 1 hydroxyl substituent,
   and
   where in the cyclobutyl ring one carbon is replaced by NR^{S}, and where the cyclobutyl may be substituted by 1 substituent selected from the group consisting of methyl and phenyl,
      where the phenyl may be substituted by 1 substituent selected from the group consisting of fluorine and methoxy,
      and
      R⁵ represents hydrogen or methyl,
   and
   L represents C₂-C₄-alkanediyl,
      where alkanediyl may be substituted by 1 hydroxyl substituent, and additionally by up to 3 fluorine atoms,
   R^{E} represents phenyl,
      where phenyl may be substituted by 1 substituent selected from the group consisting of fluorine, chlorine, hydroxyl, methoxy, trifluoromethyl, trifluoromethoxy and difluoromethoxy.

Preference is also given to compounds of the formula (I) in which
R¹ represents 1-cyclopropylethyl, 1-hydroxypropan-2-yl, 3,3,3-trifluoro-2-hydroxpropan-1-yl, 3,3-difluoro-2-hydroxpropan-1-yl, 4,4,4-trifluoro-3-hydroxy-butan-1-yl, 3-(4-fluorophenyl)-1-methylazetidin-3-yl, 3-(4-methoxyphenyl)-1-methylazetidin-3-yl or the group -L-R^{E},
   where
   L represents an C₁-C₄-alkanediyl selected from the group consisting of 1-hydroxycyclopropyl-methan-1,1,-diyl [—CH(1-hydroxycyclopropyl)-], ethan-1,1-diyl [—CH(CH₃)—], 2-hydroxy-ethan-1,1-diyl [—CH(CH₂OH)—], 3-hydroxy-propan-1,1-diyl {—CH[(CH₂)₂OH]—}, 2-hydroxy-2-methylpropan-1,1-diyl {—CH[C(CH₃)₂OH]—} and 2,2-difluoro-3-hydroxy-propan-1,1-diyl [—CH(CF₂CH₂OH)—],
   R^{E} represents phenyl,
      where phenyl may be substituted in 4-position by fluorine, hydroxyl, methoxy or trifluoromethyl, or where phenyl may be disubstituted in 3- and 4-position by fluorine.

Preference is also given to compounds of the formula (I) in which
R¹ represents 1-cyclopropylethyl, 1-hydroxypropan-2-yl, 3,3,3-trifluoro-2-hydroxpropan-1-yl, 3-(4-fluorophenyl)-1-methylazetidin-3-yl, 3-(4-methoxyphenyl)-1-methylazetidin-3-yl or the group -L-R^{E},
   where
   L represents an C₂-C₄-alkanediyl selected from the group consisting of ethan-1,1-diyl [—CH(CH₃)—], 2-hydroxy-ethan-1,1-diyl [—CH(CH₂OH)—], 3-hydroxy-propan-1,1-diyl {—CH[(CH₂)₂OH]—}, 2-hydroxy-2-methylpropan-1,1-diyl {—CH[C(CH₃)₂OH]—} and 2,2-difluoro-3-hydroxy-propan-1,1-diyl [—CH(CF₂CH₂OH)—],
   R^{E} represents phenyl,
      where phenyl may be substituted in 4-position by fluorine, hydroxyl, methoxy or trifluoromethyl.

Preference is also given to compounds of the formula (I) in which R² represents a group R²-A^{A} or R²-B or R²-C or R²-D or R²-E or R²-F, wherein
# is the point of attachment to the pyrazinone ring,
Q¹ represents CR^{8A} or N,
Q² represents CR⁸ or N,
R⁶ represents hydrogen, fluorine, chlorine, methyl, ethyl, difluoromethyl, trifluoromethyl, methoxy, trifluoromethoxy or cyclopropyl,
R⁷ represents hydrogen, fluorine, chlorine, methyl, ethyl, difluoromethyl, trifluoromethyl, methoxy, trifluoromethoxy or cyclopropyl,
with the proviso, that at least one or R⁶ and R⁷ is not hydrogen,
R^{7A} represents hydrogen, fluorine or chlorine.
R⁸ represents hydrogen, fluorine or chlorine,
R^{8A} represents hydrogen, fluorine or chlorine,
X represents CH or N,
Y represents CH or N,
Z represents CR⁹ or N,
   where at most one of X, Y and Z is N,
R⁹ represents hydrogen, fluorine, chlorine, methyl or ethyl,
A¹ represents CH₂, O or NMe,
A² represents CH₂, O or NMe,
A³ represents CH₂ or O,
where both A¹ and A² are different from O, if A³ is O,
R¹⁰ represents hydrogen or fluorine,
R¹¹ represents methyl.

Preference is also given to compounds of the formula (I) in which R² represents a group R²-A or R²-B or R²-C or R²-D or R²-E or R²-F, wherein
# is the point of attachment to the pyrazinone ring,
Q¹ represents CH or N,
Q² represents CR⁸ or N,
R⁶ represents fluorine, chlorine, methyl, ethyl, trifluoromethyl or cyclopropyl,
R⁷ represents fluorine, chlorine, methyl, ethyl, trifluoromethyl or cyclopropyl,
R⁸ represents hydrogen, fluorine or chlorine,
X represents CH or N,
Y represents CH or N,
Z represents CR⁹ or N,
   where at most one of X, Y and Z is N,
   R⁹ represents hydrogen, fluorine, chlorine, methyl or ethyl,
A¹ represents CH₂, O or NMe,
A² represents CH₂, O or NMe,
A³ represents CH₂ or O,
   where both A^{l} and A² are different from O, if A³ is O,
R¹⁰ represents hydrogen or fluorine.
R¹¹ represents methyl.

Preference is also given to compounds of the formula (I) in which R² represents a group R²-A^{A} or R²-B or R²-C, wherein
# is the point of attachment to the pyrazinone ring,
Q¹ represents CR^{8A},
Q² represents CR⁸,
R⁶ represents hydrogen, fluorine, chlorine, methyl, difluoromethyl, trifluoromethyl methoxy or trifluoromethoxy,
R⁷ represents hydrogen, fluorine, chlorine, methyl, difluoromethyl, trifluoromethyl, methoxy or trifluoromethoxy,
with the proviso, that at least one or R⁶ and R⁷ is not hydrogen,
R^{7A} represents hydrogen, fluorine or chlorine.
R⁸ represents hydrogen or fluorine,
R^{8A} represents hydrogen or fluorine,
X represents CH or N,
Y represents CH or N,
Z represents CR⁹,
   where at most one of X and Y is N,
   R⁹ represents hydrogen or methyl,
A¹ and A² represent at the same time CH₂ or O, or
one of A¹ and A² represents O and the other represents NMe,
A³ represents CH₂,
R¹⁰ represents hydrogen.

Preference is also given to compounds of the formula (I) in which R² represents a group R²-A or R²-B or R²-C, wherein
# is the point of attachment to the pyrazinone ring,
Q¹ represents CH,
Q² represents CR⁸,
R⁶ represents fluorine, chlorine, methyl or trifluoromethyl,
R⁷ represents fluorine, chlorine, methyl or trifluoromethyl,
R⁸ represents hydrogen or fluorine,
X represents CH or N,
Y represents CH or N,
Z represents CR⁹,
   where at most one of X and Y is N,
   R⁹ represents hydrogen or methyl,
A¹ and A² represent at the same time CH₂ or O, or
one of A¹ and A² represents O and the other represents NMe,
A³ represents CH₂,
   R¹⁰ represents hydrogen.

Preference is also given to compounds of the formula (I) in which R² represents 4-chlorophenyl, 3-chlorophenyl, 4-chloro-3-fluorophenyl, 3-chloro-4-fluorophenyl, 4-chloro-3-methylphenyl, 3-chloro-4-methylphenyl, 4-chloro-3-difluoromethyl-phenyl, 3-chloro-4-difluoromethyl-phenyl, 4-fluoro-3-methoxyphenyl, 3-fluoro-4-methoxyphenyl, 4-chloro-3-methoxyphenyl, 3-chloro-4-methoxyphenyl, 4-chloro-3-trifluoromethoxy-phenyl, 3-chloro-4-trifluoromethoxy-phenyl, 3,4-dichlorophenyl, 3,4-di-methylphenyl, 3-fluoro-4-methylphenyl, 4-fluoro-3-methylphenyl, 4-methyl-3-trifluoromethylphenyl, 3-methyl-4-trifluoromethylphenyl, 2,3-difluoro-4-methylphenyl, 2-fluoro-3,4-dimethylphenyl, 4-chloro-3,5-difluoropheynl, 4-chloro-2-fluoro-5-methyl, 4,5-dichloro-2-fluorophenyl, 4-chloro-2,5-difluorophenyl, or represents the group R²-B, wherein # is the point of attachment to the pyrazinone ring and where either X, Y and Z all represent CH or where X is CH, Y is N, Z is CR⁹ and R⁹ is methyl, or represents the group R²-C, wherein A¹ is O, A² is O, A³ is CH₂ and R¹⁹ is hydrogen.

Preference is also given to compounds of the formula (I) in which R² represents 4-chloro-3-methylphenyl, 3-chloro-4-methylphenyl, 3,4-dimethylphenyl, 3-fluoro-4-methylphenyl, 4-fluoro-3-methylphenyl, 4-methyl-3-trifluoromethylphenyl, 3-methyl-4-trifluoromethylphenyl, 2,3-difluoro-4-methylphenyl, 2-fluoro-3,4-dimethylphenyl, or represents the group R²-B, wherein # is the point of attachment to the pyrazinone ring and where either X, Y and Z all represent CH or where X is CH, Y is N, Z is CR⁹ and R⁹ is methyl, or represents the group R²-C, wherein A¹ is O, A² is O, A³ is CH₂ and R¹⁹ is hydrogen.

Preference is also given to compounds of the formula (I) in which R² represents 4-chlorophenyl, 3-chlorophenyl, 4-chloro-3-fluorophenyl, 3-chloro-4-fluorophenyl, 4-chloro-3-methylphenyl, 3-chloro-4-methylphenyl, 3,4-dichlorophenyl, 3,4-dimethylphenyl, 3-fluoro-4-methylphenyl, 4-methyl-3-trifluoromethylphenyl, 3-methyl-4-trifluoromethylphenyl or 4-chloro-2,5-difluorophenyl.

Preference is also given to compounds of the formula (I) in which R² represents 4-chloro-3-methylphenyl, 3-chloro-4-methylphenyl, 3,4-dimethylphenyl, 3-fluoro-4-methylphenyl, 4-methyl-3-trifluoromethylphenyl or 3-methyl-4-trifluoromethylphenyl.

Preference is also given to compounds of the formula (I) in which R² represents a group R²-A^{A}, wherein
# is the point of attachment to the pyrazinone ring,
Q¹ represents CR^{8A} or N,
Q² represents CR⁸ or N,
R⁶ represents hydrogen, fluorine, chlorine, methyl, ethyl, difluoromethyl, trifluoromethyl, methoxy, trifluoromethoxy or cyclopropyl,
R⁷ represents hydrogen, fluorine, chlorine, methyl, ethyl, difluoromethyl, trifluoromethyl, methoxy, trifluoromethoxy or cyclopropyl,
with the proviso, that at least one or R⁶ and R⁷ is not hydrogen,
R^{7A} represents hydrogen, fluorine or chlorine.
R⁸ represents hydrogen, fluorine or chlorine,
R^{8A} represents hydrogen, fluorine or chlorine.

Preference is also given to compounds of the formula (I) in which R² represents a group R²-A, wherein
# is the point of attachment to the pyrazinone ring,
Q¹ represents CH,
Q² represents CR⁸,
R⁶ represents fluorine, chlorine, methyl, ethyl or trifluoromethyl,
R⁷ represents fluorine, chlorine, methyl, ethyl or trifluoromethyl,
R⁸ represents hydrogen, fluorine or chlorine.

Preference is also given to compounds of the formula (I) in which R² represents a group R²-A^{A}, wherein
# is the point of attachment to the pyrazinone ring,
Q¹ represents CR^{8A},
Q² represents CR⁸,
R⁶ represents hydrogen, fluorine, chlorine, methyl, difluoromethyl, trifluoromethyl methoxy or trifluoromethoxy,
R⁷ represents hydrogen, fluorine, chlorine, methyl, difluoromethyl, trifluoromethyl, methoxy or trifluoromethoxy,
with the proviso, that at least one or R⁶ and R⁷ is not hydrogen,
R^{7A} represents hydrogen, fluorine or chlorine.
R⁸ represents hydrogen or fluorine,
R^{8A} represents hydrogen or fluorine.

Preference is also given to compounds of the formula (I) in which R² represents a group R²-A, wherein
# is the point of attachment to the pyrazinone ring,
Q¹ represents CH,
Q² represents CR⁸,
R⁶ represents fluorine, chlorine, methyl or trifluoromethyl,
R⁷ represents fluorine, chlorine, methyl or trifluoromethyl,
R⁸ represents hydrogen or fluorine.

Preference is also given to compounds of the formula (I) in which R² represents 4-chlorophenyl, 3-chlorophenyl, 4-chloro-3-fluorophenyl, 3-chloro-4-fluorophenyl, 4-chloro-3-methylphenyl, 3-chloro-4-methylphenyl, 4-chloro-3-difluoromethyl-phenyl, 3-chloro-4-difluoromethyl-phenyl, 4-fluoro-3-methoxyphenyl, 3-fluoro-4-methoxyphenyl, 4-chloro-3-methoxyphenyl, 3-chloro-4-methoxyphenyl, 4-chloro-3-trifluoromethoxy-phenyl, 3-chloro-4-trifluoromethoxy-phenyl, 3,4-dichlorophenyl, 3,4-di-methylphenyl, 3-fluoro-4-methylphenyl, 4-fluoro-3-methylphenyl, 4-methyl-3-trifluoromethylphenyl, 3-methyl-4-trifluoromethylphenyl, 2,3-difluoro-4-methylphenyl, 2-fluoro-3,4-dimethylphenyl, 4-chloro-3,5-difluoropheynl, 4-chloro-2-fluoro-5-methyl, 4,5-dichloro-2-fluorophenyl, 4-chloro-2,5-difluorophenyl.

Preference is also given to compounds of the formula (I) in which R² represents 4-chloro-3-methylphenyl, 3-chloro-4-methylphenyl, 3,4-dimethylphenyl, 3-fluoro-4-methylphenyl, 4-fluoro-3-methylphenyl, 4-methyl-3-trifluoromethylphenyl, 3-methyl-4-trifluoromethylphenyl, 2,3-difluoro-4-methylphenyl, 2-fluoro-3,4-dimethylphenyl.

Preference is also given to compounds of the formula (I) in which R² represents a group R²-B, wherein
# is the point of attachment to the pyrazinone ring,
X represents CH or N,
Y represents CH or N,
Z represents CR⁹ or N,
   where at most one of X, Y and Z is N,
   R⁹ represents hydrogen, fluorine, chlorine, methyl or ethyl.

Preference is also given to compounds of the formula (I) in which R² represents a group R²-B, wherein
# is the point of attachment to the pyrazinone ring,
X represents CH or N,
Y represents CH or N,
Z represents CR⁹,
   where at most one of X and Y is N,
   R⁹ represents hydrogen or methyl.

Preference is also given to compounds of the formula (I) in which R² represents the group R²-B, wherein # is the point of attachment to the pyrazinone ring and where either X, Y and Z all represent CH or where X is CH, Y is N, Z is CR⁹ and R⁹ is methyl.

Preference is also given to compounds of the formula (I) in which R² represents a group R²-C, wherein
# is the point of attachment to the pyrazinone ring,
A¹ represents CH₂, O or NMe,
A² represents CH₂, O or NMe,
A³ represents CH₂ or O,
   where both A¹ and A² are different from O, if A³ is O,
R10 represents hydrogen or fluorine.

Preference is also given to compounds of the formula (I) in which R² represents a group R²-C, wherein
# is the point of attachment to the pyrazinone ring,
A¹ and A² represent at the same time CH₂ or O, or
one of A¹ and A² represents O and the other represents NMe,
A³ represents CH₂,
R¹⁰ represents hydrogen.

Preference is also given to compounds of the formula (I) in which R² represents the group R²-C, wherein A¹ is O, A² is O, A³ is CH₂ and R¹⁰ is hydrogen.

Preference is also given to compounds of the formula (I) in which R³ represents hydrogen, fluorine, chlorine, cyano, methyl or ethyl.

Preference is also given to compounds of the formula (I) in which R³ represents hydrogen, fluorine, chlorine or methyl.

Preference is also given to compounds of the formula (I) in which R³ represents hydrogen, fluorine or methyl.

Preference is also given to compounds of the formula (I) in which R³ represents hydrogen.

Preference is also given to compounds of the formula (I) in which R⁴ represents hydrogen, fluorine, chlorine, methyl, ethyl, n-propyl, isopropyl, difluoromethyl, trifluoromethyl, pentafluoroethyl, cyclopropyl, 2,2-difluorocyclopropan-1-yl, cyclobutyl, 3,3-difluorocyclobutan-1-yl, piperidin-4-yl, cyano or methoxymethyl.

Preference is also given to compounds of the formula (I) in which R⁴ represents hydrogen, fluorine, chlorine, methyl, ethyl, n-propyl, isopropyl, difluoromethyl, trifluoromethyl, cyclopropyl, piperidin-4-yl, cyano or methoxymethyl.

Preference is also given to compounds of the formula (I) in which R⁴ represents hydrogen, chlorine, methyl, isopropyl, trifluoromethyl, pentafluoroethyl, cyclopropyl, 2,2-difluorocyclopropan-1-yl, cyclobutyl, 3,3-difluorocyclobutan-1-yl, cyano or methoxymethyl.

Preference is also given to compounds of the formula (I) in which R⁴ represents hydrogen, chlorine, methyl, isopropyl, trifluoromethyl, cyclopropyl, cyano or methoxymethyl.

Preference is also given to compounds of the formula (I) in which R⁴ represents hydrogen, methyl, isopropyl, trifluoromethyl or cyclopropyl.

Preference is also given to compounds of the formula (I) in which R⁴ represents hydrogen, methyl, trifluoromethyl or cyclopropyl.

Preference is also given to compounds of the formula (I) in which R⁴ represents isopropyl, trifluoromethyl or cyclopropyl.

Preference is also given to compounds of the formula (I) in which R⁴ represents trifluoromethyl or cyclopropyl.

Preference is also given to compounds of the formula (I) in which R³ represents hydrogen, fluorine or methyl and R⁴ represents hydrogen, methyl, isopropyl, trifluoromethyl or cyclopropyl.

Preference is also given to compounds of the formula (I) in which R³ represents hydrogen, fluorine or methyl and R⁴ represents hydrogen, methyl, trifluoromethyl or cyclopropyl.

Preference is also given to compounds of the formula (I) in which R³ represents hydrogen and R⁴ represents isopropyl, trifluoromethyl or cyclopropyl.

Preference is also given to compounds of the formula (I) in which R³ represents hydrogen and R⁴ represents trifluoromethyl or cyclopropyl.

The invention further provides a method for preparing compounds of the formula (I), or salts thereof, solvates thereof or solvates of the salts thereof, wherein
[A] the compounds of the formula (II)

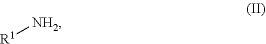

in which R¹ is as defined above,
are reacted with compounds of the formula (III)

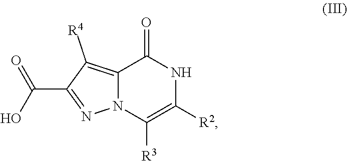

in which R², R³ and R⁴ are as defined above,
in the presence of a dehydrating agent to give compounds of the formula (I).

The reaction [A] is generally carried out in inert solvents, if appropriate in the presence of a base, preferably in a temperature range from 0° C. to 50° C. at atmospheric pressure.

Alternatively, the reaction [A] can also be carried out without a solvent only in one base if the base is a liquid at room temperature.

Suitable dehydrating agents here are, for example, carbodiimides such as N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimide-N′-propyloxymethyl-polystyrene (PS-carbodiimide) or carbonyl compounds such as carbonyldiimidazole (CDI), or 1,2-oxazolium compounds such as 2-ethyl-5-phenyl-1,2-oxazolium 3-sulphate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroformate, or bis-(2-oxo-3-oxazolidinyl)phosphoryl chloride or benzotriazolyloxytri(dimethylamino)-phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluoro-phosphate (HATU), or 1-hydroxybenzotriazole (HOBt), or benzotriazol-1-yloxytris(dimethyl-amino)phosphonium hexafluorophosphate (BOP), or ethyl cyano(hydroxyimino)acetate (Oxyma), or (1-cyano-2-ethoxy-2-oxoethylidenamino-oxy)dimethylamino-morpholino-carbenium hexafluorophosphate (COMU), or N-[(dimethylamino)-(3H-[1,2,3]triazolo[4,5-b]pyridin-3-yloxy)methylidene]-N-methylmethanaminium hexafluorophosphate, or 2,4,6-tripropyl-1,3,5,2,4,6-trioxatriphosphinane-2,4,6-trioxide (T3P), or mixtures of these with bases, the condensation with HATU, CDI or with EDC being preferred.

Bases are, for example, alkali metal carbonates such as sodium carbonate or potassium carbonate, or sodium bicarbonate or potassium bicarbonate, or organic bases such as trialkylamines, for example triethylamine, N-methylmorpholine, N-methylpiperidine, 4-dimethylaminopyridine or diisopropylethylamin, or pyridine; preference is given to condensation with diisopropylethylamine, N-methylmorpholine or 4-dimethylaminopyridine.

Inert solvents are, for example, halogenated hydrocarbons such as dichloromethane or trichloromethane, hydrocarbons such as benzene or toluene, or other solvents such as nitromethane, dioxane, diethyl ether, tetrahydrofuran, ethyl acetate, dimethylformamide, dimethylacetamide, dimethyl sulphoxide, N-methylpyrrolidone or acetonitrile, or mixtures of the solvents, preference being given to dimethylformamide or N-methylpyrrolidone.

The compounds of the formula (II) are known or can be synthesized from the corresponding starting compounds by known processes.

The compounds of the formula (III) are known or can be prepared
[B] by reacting compounds of the formula (IV)

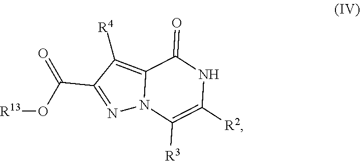

in which
R², R³ and R⁴ are as defined above and
R¹³ represents methyl, ethyl, propyl, isopropyl, tert-butyl or benzyl, with a base.

The reaction [B] is generally carried out in inert solvents, preferably in a temperature range from room temperature up to reflux of the solvents at atmospheric pressure.

Inert solvents are, for example, halogenated hydrocarbons such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvents with water, preference being given to a mixture of tetrahydrofuran and water or ethanol and water.

Bases are, for example, alkali metal hydroxides such as sodium hydroxide, lithium hydroxide or potassium hydroxide, or alkali metal carbonates such as caesium carbonate, sodium carbonate or potassium carbonate, or alkoxides such as potassium tert-butoxide or sodium ted-butoxide, preference being given to lithium hydroxide or sodium hyroxide.

In case where R^{4a} represents chlorine, bromine or iodine compounds of the formula (IV-a)

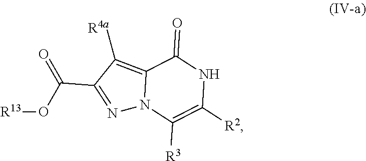

in which R², R³ and R¹³ are as defined above
[C] may be reacted with copper(l)cyanide to obtain compounds of the formula (IV-b)

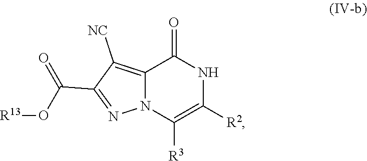

in which R², R³ and R¹³ are as defined above,
or
[D] may be reacted with cyclopropylzinc bromide in the presence of a palladium source and a suitable ligand to obtain compounds of the formula (IV-c)

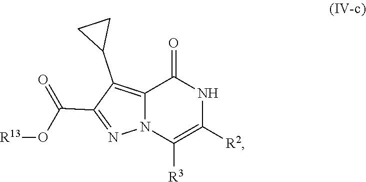

in which R², R³ and R¹³ are as defined above.

The reaction [C] is generally carried out in inert solvents, preferably in a temperature range from room temperature up to reflux of the solvents at atmospheric pressure. Preferred solvents are dimethylformamide, dimethylacetamide and N-methylpyrrolidone.

The reaction [D] is generally carried out in inert solvents, preferably in a temperature range from room temperature up to reflux of the solvents at atmospheric pressure.

The preferred solvent is tetrahydrofuran and the preferred catalyst is [(2-dicyclohexylphosphino-2′,6′-bis(N,N-dimethylamino)-1,1′-biphenyl)-2-(2′-amino-1,1-biphenyl)] palladium(II) methanesulfonate (CPhos Pd G3).

The processes described in reations [C] and [D] may analoguesly applied to compounds of formulae (I), (Ill), (V) and (VI). If R⁴ in these formulae is replaced by R^{4a} the described transformation into the cyano or cyclopropyl group may be carried out accordingly.

The compounds of the formula (IV) are known or can be prepared
[E] by reacting compounds of the formula (V)

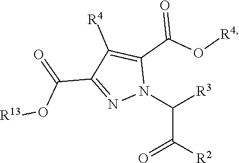

in which
R², R³ and R⁴ are each as defined above and
R¹³ and R¹⁴ represent independently of each other methyl, ethyl, propyl, isopropyl, tert-butyl or benzyl,
with an ammonia equivalent in the presence of an acid.

The reaction [E] is generally carried out in inert solvents, preferably in a temperature range from room temperature up to reflux of the solvents at atmospheric pressure.

Alternatively, the reaction [E] can also be carried out without a solvent only in one acid if the acid is a liquid at room temperature.

Ammonia equivalents are, for example, ammonium acetate, ammonium formate, ammonium propionate, or ammonium chloride, preference being given to ammonium acetate.

Acids are, for example, organic acids such was formic acid, acetic acid, propionic acid, trifluoroacetic acid, benzenesulfonic acid, toluenesulfonic acid, trifluoromethanesulfonic acid, or mineral acids such as, for example, hydrogen chloride, or hydrogen bromide, preference being given to acetic acid.

The compounds of the formula (V) are known or can be prepared
[F] by reacting compounds of the formula

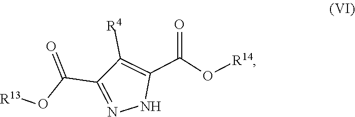

in which
R⁴ is as defined above and
R¹³ and R¹⁴ represent independently of each other methyl, ethyl, propyl, isopropyl, tert-butyl or benzyl
with compounds of the formula

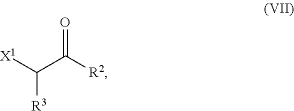

in which
R² and R³ are each as defined above and
X¹ represent chlorine, bromine or iodine, triflate,
   in the prexence of a base.

The reaction [F] is generally carried out in inert solvents, preferably in a temperature range from room temperature up to reflux of the solvents at atmospheric pressure.

Inert solvents are, for example, halogenated hydrocarbons such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, N-methyl-pyrrolidine, dimethylacetamide, acetonitrile, acetone or pyridine, or mixtures of solvents, or mixtures of solvents with water, preference being given to acetone.

Bases are, for example, alkali metal carbonates such as sodium carbonate or potassium carbonate, or sodium bicarbonate or potassium bicarbonate, or organic bases such as trialkylamines, for example triethylamine, N-methylmorpholine, N-methylpiperidine, 4-dimethylaminopyridine or diisopropylethylamin, or pyridine, or other bases such as sodium hydride, or lithium diisopropylamide; preference is given to potassium carbonate.

Optionally catalytic amounts of iodides such as, for example, sodium iodide or tetrabutylammonium iodide can be added.

The compounds of the formula (VI) are known or can be prepared
[G] by reacting compounds of the formula

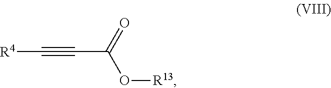

in which
R⁴ is as defined above and
R¹³ represents methyl, ethyl, propyl, isopropyl, tert-butyl or benzyl,
with compounds of the formula

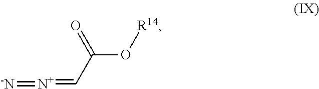

in which R¹⁴ represents methyl, ethyl, propyl, isopropyl, tert-butyl or benzyl.

The reaction [G] is generally carried out in inert solvents, preferably at temperature range from 0° C. up to reflux of the solvents at atmospheric pressure.

Inert solvents are, for example, halogenated hydrocarbons such as dichloromethane, trichloromethane, carbon tetrachloride or 1 ,2-dichloroethane, ethers such as diethyl ether, methyl tertbutyl ether, 1,2-dimethoxyethane, dioxane or tetrahydrofuran, or other solvents such as toluene.

The reaction [G] can also be carried out without a solvent.

Alternatively, the compounds of the formula (VI) can be prepared
[H] by reacting compounds of the formula

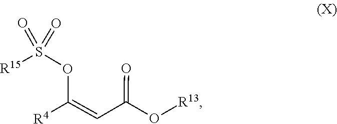

in which
R⁴ is as defined above,
R¹³ represents methyl, ethyl, propyl, isopropyl, tert-butyl or benzyl, and
R¹⁵ represents methyl, trifluoromethyl or 4-methylphenyl,
with compounds of the formula

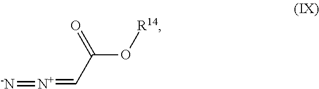

in which R¹⁴ represents methyl, ethyl, propyl, isopropyl, tert-butyl or benzyl
in the presence of a palladium source and a suitable ligand and a base.

The reaction [H] is generally carried out in inert solvents, preferably at temperature range from room temperature up to reflux of the solvents at atmospheric pressure.

Preferred inert solvents are, for example dimethylformamide, N-methyl-pyrrolidine, dimethylacetamide or acetonitrile.

Bases are, for example, organic bases such as trialkylamines, for example triethylamine, N-methylmorpholine, N-methylpiperidine, 4-dimethylamino-pyridine or diisopropylethylamin, or pyridine, preference being given to N-methylmorpholine.

The preferred catalyst is tetrakis(triphenylphosphin)palladium(0).

The compounds of the formula (X) are known or can be prepared
[I] by reacting compounds of the formula (XI), which are known or commercially available

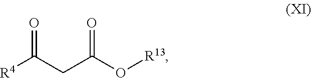

in which
R⁴ is as defined above and
R¹³ represents methyl, ethyl, propyl, isopropyl, tert-butyl or benzyl,
with a sulfonic anhydride (e.g. trifluoromethanesulfonic anhydride) or sulfonic chloride (e.g. methylsulfonyl chloride, 4-toluenesulfonyl chloride) in the presence of a base.

The reaction [I] is generally carried out in inert solvents, preferably at temperature range from 0° C. up to room temperature.

Inert solvents are, for example, halogenated hydrocarbons such as dichloromethane, trichloromethane, carbon tetrachloride or 1,2-dichloroethane, ethers such as diethyl ether, methyl tert-butyl ether, 1,2-dimethoxyethane, dioxane or tetrahydrofuran, or other solvents such as dimethylformamide, N-methyl-pyrrolidine, dimethylacetamide, acetonitrile or toluene, or mixtures of solvents, or mixtures of solvents with water, preference being given to toluene-water mixtures.

Sulfonic chlorides are, for example, methanesulfonic chloride and p-toluenesulfonyl chloride, sulfonic anhydrides are, for example, trifluoromethanesulfonic anhydride-, preference being given to trifluoromethanesulfonic anhydride.

Bases are, for example, alkali metal carbonates such as sodium carbonate or potassium carbonate, or sodium bicarbonate or potassium bicarbonate, or alkali metal hydroxides, like lithium or sodium hydroxides, or organic bases such as trialkylamines, for example triethylamine, N-methylmorpholine, N-methylpiperidine, 4-dimethylamino-pyridine, diisopropylethylamine or tetramethylammonium hydroxide, or pyridine, or other bases such as sodium hydride, or lithium diisopropylamide; preference is given to lithium hydroxide.

The compounds of the formula (VII) are known or can be synthesized from the corresponding starting compounds by known processes.

The preparation of the starting compounds and of the compounds of the formula (I) can be illustrated by the synthesis schemes which follow.

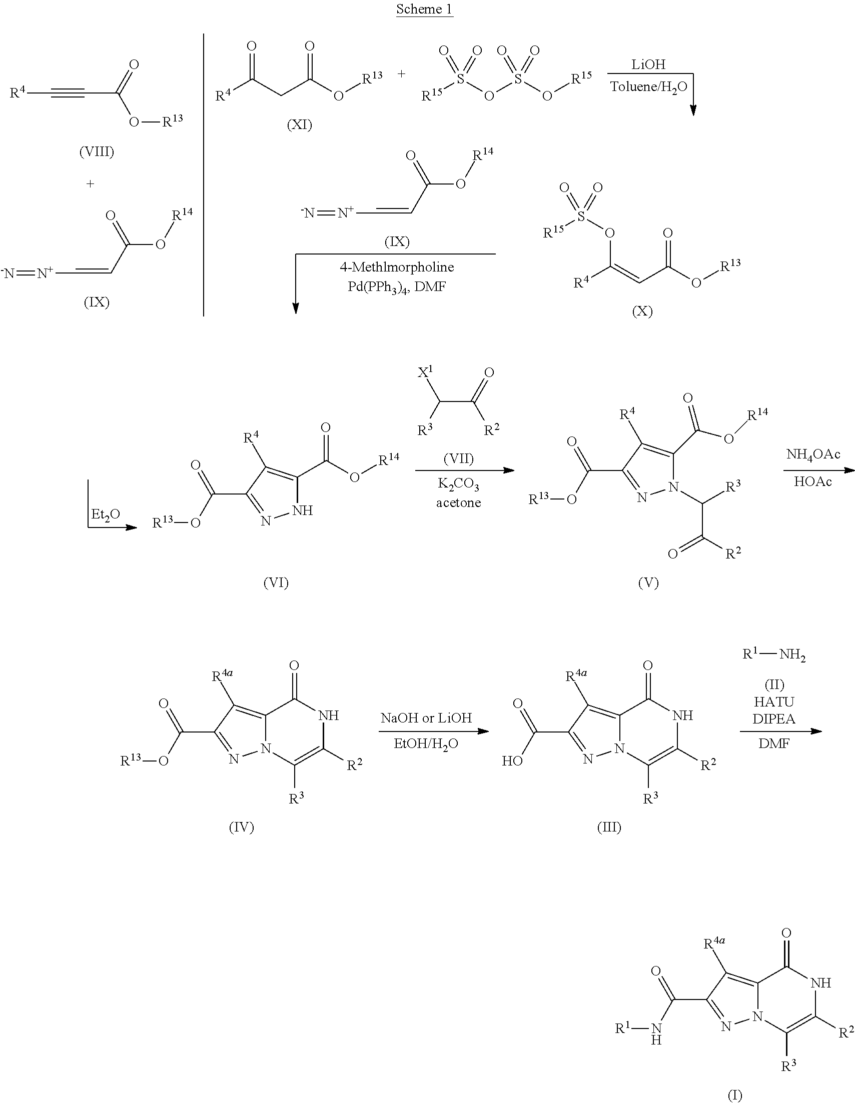

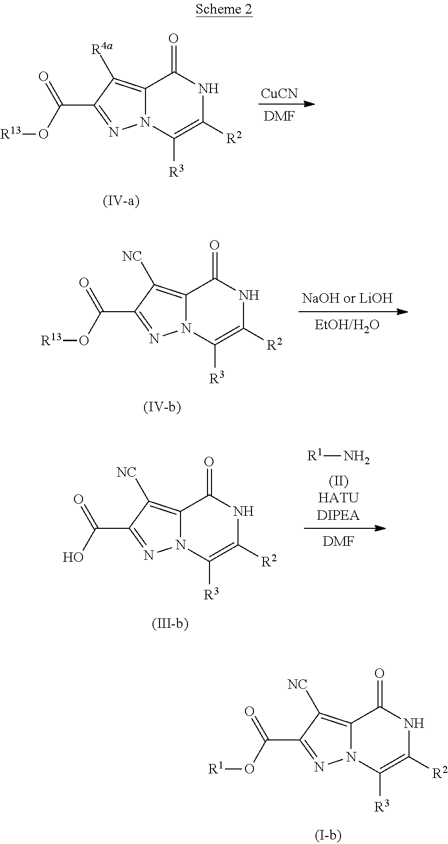

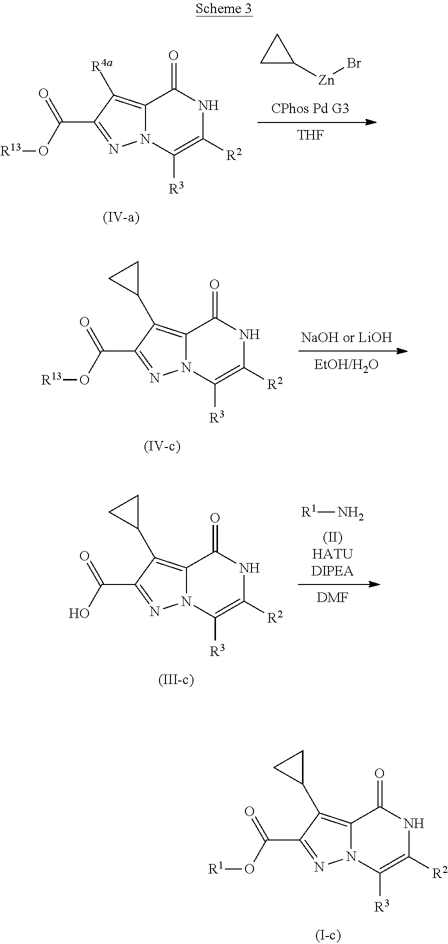

Formulae (III-b), (III-c), (I-b) and (I-c) are subgroups of the formulae (III) and (I), respectively, obtained by transformation of the compounds of formulae (IV-b) and (IV-c).

The compounds of the invention have valuable pharmacological properties and can be used for prevention and treatment of diseases in humans and animals.

The compounds according to the invention have an unforeseeable useful pharmacological activity spectrum and good pharmacokinetic behavior, in particular a sufficient exposure of such a compound in the blood above the minimal effective concentration within a given dosing interval after oral administration. Such a profile results in an improved peak-to-trough ratio (quotient of maximum to minimum concentration) within a given dosing interval, which has the advantage that the compound can be administered less frequently and at a significantly lower dose to achieve an effect. They are compounds that inhibit the activation of the EP3 receptor by its ligand Prostaglandin E2 (PGE2).

They are therefore suitable for use as medicaments for the treatment and/or prophylaxis of diseases in humans and animals.

The present invention further provides for the use of the compounds according to the invention for the treatment and/or prophylaxis of disorders, in particular cardiovascular disorders, preferably thrombotic or thromboembolic disorders and/or thrombotic or thromboembolic complications such as acute coronary syndrome or myocardial infarction or ischemic stroke or peripheral arterial occlusive disease , and/or diabetes, and/or ophthalmic disorders and/or urogenital disorders, in particular those associated with excess PGE2.

Increased PGE2 concentrations have been measured in atherosclerotic vascular walls of mice and humans. Once released upon plaque rupture, PGE2 binds on four specific receptors EP1, EP2, EP3 and EP4 on cell membranes. PGE2 has been shown to interfere with human and murine platelet function via EP3 and EP4 receptors. Stimulation of EP3 potentiates platelet activation and aggregation induced by primary agonists like collagen or ADP, whereas stimulation of EP4 inhibits platelet activation. This PGE2-dependent balance of platelet activation and inhibition can be tipped by modulation of EP3 or EP4 receptors.

In contrast to established platelet antagonists, for example COX inhibitors like acetyl salicylic acid (Aspirin®) and P2Y12 receptor antagonists like clopidogrel, prasugrel or ticagrelor, modulation of platelet activity via the EP3 receptor does not interfere with physiologic hemostasis and therefore is not expected to induce or increase the risk of bleeding.

Therefore, blocking the EP3 receptor by specific antagonists should be a beneficial strategy for prevention and treatment of atherothrombosis by local abrogation of platelet activation without altering hemostasis.

In patients suffering from PAOD, chronically inflamed vessel walls produce PGE2 to activate EP3 receptors not only on platelets but also on vascular smooth muscle cells thus preventing microvascular relaxation and contributing to malperfusion of peripheral tissues. Therefore, an antagonist to the EP3 receptor might be expected to provide therapeutic benefit specifically in PAOD.

For the purpose of the present invention, the “thrombotic or thromboembolic disorders” include disorders which occur preferably in the arterial vasculature and which can be treated with the compounds according to the invention, in particular disorders leading to peripheral arterial occlusive disorders and in the coronary arteries of the heart, such as acute coronary syndrome (ACS), myocardial infarction with ST segment elevation (STEMI) and without ST segment elevation (non-STEMI), stable angina pectoris, unstable angina pectoris, reocclusions and restenoses after coronary interventions such as angioplasty, stent implantation or aortocoronary bypass, but also thrombotic or thromboembolic disorders in further vessels leading to ischemic stroke and—transitory ischaemic attacks.

Moreover, the compounds according to the invention are suitable in particular for the treatment and/or prophylaxis of disorders where, the pro-inflammatory component also plays an essential role.

The present invention further provides for the use of the compounds according to the invention for the treatment and/or prophylaxis of disorders, especially the disorders mentioned above.

The present invention further provides for the use of the compounds according to the invention for production of a medicament for the treatment and/or prophylaxis of disorders, especially the disorders mentioned above.

The present invention further provides a method for the treatment and/or prophylaxis of disorders, especially the disorders mentioned above, using a therapeutically effective amount of a compound according to the invention.

The present invention further provides the compounds according to the invention for use in a method for the treatment and/or prophylaxis of disorders, especially the disorders mentioned above, using a therapeutically effective amount of a compound according to the invention.

Particularly the present invention provides the compounds according to the invention for use in a method for the treatment and/or prophylaxis of thrombotic or thromboembolic, in particular atherothrombotic disorders using a therapeutically effective amount of a compound according to the invention.

The present invention further provides medicaments comprising a compound according to the invention and one or more further active compounds.

In addition, the compounds according to the invention can also be used for preventing coagulation ex vivo, for example for the protection of organs to be transplanted against organ damage caused by formation of clots and for protecting the organ recipient against thromboemboli from the transplanted organ, for preserving blood and plasma products, for cleaning/pretreating catheters and other medical auxiliaries and instruments, for coating synthetic surfaces of medical auxiliaries and instruments used in vivo or ex vivo or for biological samples which may comprise factor XIa or plasma kallikrein.

The present invention furthermore provides a method for preventing the coagulation of blood in vitro, in particular in banked blood or biological samples which may comprise factor XIa or plasma kallikrein or both enzymes, which method is characterized in that an anticoagulatory effective amount of the compound according to the invention is added.

The present invention further provides medicaments comprising a compound according to the invention and one or more further active compounds, in particular for the treatment and/or prophylaxis of the disorders mentioned above. Preferred examples of active compounds suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilatators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors) such as, for example, tissue plasminogen activator (t-PA, for example Actilyse), streptokinase, reteplase and urokinase or plasminogen-modulating substances causing increased formation of plasmin;
anticoagulatory substances (anticoagulants) such as, for example, heparin (UFH), low-molecular-weight heparins (LMVV), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI) such as, for example, Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors such as, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux,
inhibitors of coagulation factor XI and XIa such as, for example, FXI ASO-LICA, BAY 121-3790, MAA868, BMS986177, EP-7041, AB-022,
substances which inhibit the aggregation of platelets (platelet aggregation inhibitors, thrombocyte aggregation inhibitors), such as, for example, acetylsalicylic acid (such as, for example, aspirin), P2Y12 antagonists such as, for example, ticlopidine (Ticlid), clopidogrel (Plavix), prasugrel, ticagrelor, cangrelor, elinogrel, PAR-1 antagonists such as, for example, vorapaxar, PAR-4 antagonists;
platelet adhesion inhibitors such as GPVI and/or GPIb antagonists such as, for example, Revacept or caplacizumab;
fibrinogen receptor antagonists (glycoprotein-IIb/IIIa antagonists), for example abciximab, eptifibatide, tirofiban, lamifiban, lefradafiban and fradafiban;
recombinant human activated protein C such as, for example, Xigris or recombinant thrombomudulin;
and also antiarrhythmics;
inhibitors of VEGF and/or PDGF signal paths such as, for example, aflibercept, ranibizumab, bevacizumab, KH-902, pegaptanib, ramucirumab, squalamin or bevasiranib, apatinib, axitinib, brivanib, cediranib, dovitinib, lenvatinib, linifanib, motesanib, pazopanib, regorafenib, sorafenib, sunitinib, tivozanib, vandetanib, vatalanib, Vargatef and E-10030;
inhibitors of angiopoietin-Tie signal paths such as, for example, AMG386;
inhibitors of Tie2 receptor tyrosine kinase;
inhibitors of the integrin signal paths such as, for example, volociximab, cilengitide and ALG1001;
inhibitors of the P13K-Akt-mTor signal paths such as, for example, XL-147, perifosine, MK2206, sirolimus, temsirolimus and everolimus;
corticosteroids such as, for example, anecortave, betamethasone, dexamethasone, triamcinolone, fluocinolone and fluocinolone acetonide;
inhibitors of the ALK1-Smad1/5 signal path such as, for example, ACE041;
cyclooxygenase inhibitors such as, for example, bromfenac and nepafenac;
inhibitors of the kallikrein-kinin system such as, for example, safotibant and ecallantide;
inhibitors of the sphingosine 1-phosphate signal paths such as, for example, sonepcizumab;
inhibitors of the complement-C5a receptor such as, for example, eculizumab;
inhibitors of the 5HT1a receptor such as, for example, tandospirone;
inhibitors of the Ras-Raf-Mek-Erk signal path; inhibitors of the MAPK signal paths; inhibitors of the FGF signal paths; inhibitors of endothelial cell proliferation; apoptosis-inducing active compounds;
photodynamic therapy consisting of an active compound and the action of light, the active compound being, for example, verteporfin.

“Combinations” for the purpose of the invention mean not only dosage forms which contain all the components (so-called fixed combinations) and combination packs which contain the components separate from one another, but also components which are administered simultaneously or sequentially, provided that they are used for the prophylaxis and/or treatment of the same disease. It is likewise possible to combine two or more active ingredients with one another, meaning that they are thus each in two-component or multicomponent combinations.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crys- and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Suitable for extraocular (topic) administration are administration forms which operate in accordance with the prior art, which release the active compound rapidly and/or in a modified or controlled manner and which contain the active compound in crystalline and/or amorphized and/or dissolved form such as, for example, eye drops, sprays and lotions (e.g. solutions, suspensions, vesicular/colloidal systems, emulsions, aerosols), powders for eye drops, sprays and lotions (e.g. ground active compound, mixtures, lyophilisates, precipitated active compound), semisolid eye preparations (e.g. hydrogels, in-situ hydrogels, creams and ointments), eye inserts (solid and semisolid preparations, e.g. bioadhesives, films/wafers, tablets, contact lenses).

Intraocular administration includes, for example, intravitreal, subretinal, subscleral, intrachoroidal, subconjunctival, retrobulbar and subtenon administration. Suitable for intraocular administration are administration forms which operate in accordance with the prior art, which release the active compound rapidly and/or in a modified or controlled manner and which contain the active compound in crystalline and/or amorphized and/or dissolved form such as, for example, preparations for injection and concentrates for preparations for injection (e.g. solutions, suspensions, vesicular/colloidal systems, emulsions), powders for preparations for injection (e.g. ground active compound, mixtures, lyophilisates, precipitated active compound), gels for preparations for injection (semisolid preparations, e.g. hydrogels, in-situ hydrogels) and implants (solid preparations, e.g. biodegradable and nonbiodegradable implants, implantable pumps).

Preference is given to oral administration or, in the case of ophthalmologic disorders, extraocular and intraocular administration.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopole); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragite)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprises at least one compound according to the invention, conventionally together with one or more pharmaceutically suitable excipient(s), and to their use according to the present invention.

An embodiment of the invention are pharmaceutical compositions comprising at least one compound of formula (I) according to the invention, preferably together with at least one inert, nontoxic, pharmaceutically suitable auxiliary, and the use of these pharmaceutical compositions for the above cited purposes.

In accordance with another aspect, the present invention covers pharmaceutical combinations, in particular medicaments, comprising at least one compound of general formula (I) of the present invention and at least one or more further active ingredients, in particular for the treatment and/or prophylaxis of cardiovascular disorders, preferably thrombotic or thromboembolic disorders, and diabetes, and also urogenital and ophthalmic disorders.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The inventive compounds can be employed alone or, if required, in combination with other active ingredients. The present invention further provides medicaments comprising at least one of the inventive compounds and one or more further active ingredients, especially for treatment and/or prophylaxis of the aforementioned disorders. Preferred examples of suitable active ingredient combinations include:
organic nitrates and NO donors, for example sodium nitroprusside, nitroglycerin, isosorbide mononitrate, isosorbide dinitrate, molsidomine or SIN-1, and inhaled NO;
compounds which inhibit the breakdown of cyclic guanosine monophosphate (cGMP), for example inhibitors of phosphodiesterases (PDE) 1, 2 and/or 5, especially PDE 5 inhibitors such as sildenafil, vardenafil, tadalafil, udenafil, desantafil, avanafil, mirodenafil, lodenafil or PF-00489791;
antithrombotic agents, by way of example and with preference from the group of the platelet aggregation inhibitors, the anticoagulants or the profibrinolytic substances;
hypotensive active ingredients, by way of example and with preference from the group of the calcium antagonists, angiotensin All antagonists, ACE inhibitors, NEP-inhibitors, vasopeptidase-inhibitors, endothelin antagonists, renin inhibitors, alpha-receptor blockers, beta-receptor blockers, mineralocorticoid receptor antagonists, rho-kinase-inhibitors and the diuretics;
antiarrhythmic agents, by way of example and with preference from the group of sodium channel blocker, beta-receptor blocker, potassium channel blocker, calcium antagonists, If-channel blocker, digitalis, parasympatholytics (vagoliytics), sympathomimetics and other antiarrhythmics as adenosin, adenosine receptor agonists as well as vernakalant;
positive-inotrop agents, by way of example cardiac glycoside (Dogoxin), beta-adrenergic and dopaminergic agonists, such as isoprenalin, adrenalin, noradrenalin, dopamin or dobutamin;
vasopressin-receptor-antagonists, by way of example and with preference from the group of conivaptan, tolvaptan, lixivaptan, mozavaptan, satavaptan, SR-121463, RWJ 676070 or BAY 86-8050, as well as the compounds described in WO 2010/105770, WO2011/104322 and WO 2016/071212;
active ingredients which alter lipid metabolism, for example and with preference from the group of the thyroid receptor agonists, cholesterol synthesis inhibitors such as, by way of example and preferably, HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists.
bronchodilatory agents, for example and with preference from the group of the beta-adrenergic rezeptor-agonists, such as, by way of example and preferably, albuterol, isoproterenol, metaproterenol, terbutalin, formoterol or salmeterol, or from the group of the anticholinergics, such as, by way of example and preferably, ipratropiumbromid;
anti-inflammatory agents, for example and with preference from the group of the glucocorticoids, such as, by way of example and preferably, prednison, prednisolon, methylprednisolon, triamcinolon, dexamethason, beclomethason, betamethason, flunisolid, budesonid or fluticason as well as the non-steroidal anti-inflammatory agents (NSAIDs), by way of example and preferably, acetyl salicylic acid (aspirin), ibuprofen and naproxen, 5-amino salicylic acid-derivates, leukotriene-antagonists, TNF-alpha-inhibitors and chemokin-receptor antagonists, such as CCR1, 2 and/or 5 inhibitors;
agents that inhibit the signal transductions cascade, for example and with preference from the group of the kinase inhibitors, by way of example and preferably, from the group of the tyrosine kinase- and/or serine/threonine kinase inhibitors;
agents, that inhibit the degradation and modification of the extracellular matrix, for example and with preference from the group of the inhibitors of the matrix-metalloproteases (MMPs), by way of example and preferably, inhibitors of chymasee, stromelysine, collagenases, gelatinases and aggrecanases (with preference from the group of MMP-1, MMP-3, MMP-8, MMP-9, MMP-10, MMP-11 and MMP-13) as well as of the metallo-elastase (MMP-12) and neutrophil-elastase (HNE), as for example sivelestat or DX-890;
agents, that block the bindung of serotonin to its receptor, for example and with preference antagonists of the 5-HT2b-receptor;
organic nitrates and NO-donators, for example and with preference sodium nitroprussid, nitroglycerine, isosorbid mononitrate, isosorbid dinitrate, molsidomine or SIN-1, as well as inhaled NO;
NO-independent, but heme-dependent stimulators of the soluble guanylate cyclase, for example and with preference the compounds described in WO 00/06568, WO 00/06569, WO 02/42301, WO 03/095451, WO 2011/147809, WO 2012/004258, WO 2012/028647 and WO 2012/059549;
NO-independent and heme-independent activators of the soluble guanylate cyclase, for example and with preference the compounds described in WO 01/19355, WO 01/19776, WO 01/19778, WO 01/19780, WO 02/070462 and WO 02/070510 beschriebenen Verbindungen;
agents, that stimulates the synthesis of cGMP, wie beispielsweise sGC Modulatoren, for example and with preference riociguat, cinaciguat, vericiguat or BAY 1101042;
prostacyclin-analogs, for example and with preference iloprost, beraprost, treprostinil or epoprostenol;
agents, that inhibit soulble epoxidhydrolase (sEH), for example and with preference N,N′-Dicyclohexyl urea, 12-(3-Adamantan-1-yl-ureido)-dodecanic acid or 1-Adamantan-1-yl-3-{5-[2-(2-ethoxyethoxy)ethoxy]pentyl}-urea;
agents that interact with glucose metabolism, for example and with preference insuline, biguanide, thiazolidinedione, sulfonyl urea, acarbose, DPP4 inhibitors, GLP-1 analogs or SGLT-1 inhibitors;
natriuretic peptides, for example and with preference atrial natriuretic peptide (ANP), natriuretic peptide type B (BNP, Nesiritid) natriuretic peptide type C (CNP) or urodilatin;
activators of the cardiac myosin, for example and with preference omecamtiv mecarbil (CK-1827452);
calcium-sensitizers, for example and with preference levosimendan;
agents that affect the energy metabolism of the heart, for example and with preference etomoxir, dichloroacetat, ranolazine or trimetazidine, full or partial adenosine A1 receptor agonists such as GS-9667 (formerly known as CVT-3619), capadenoson, neladenoson and neladenoson bialanate;
agents that affect the heart rate, for example and with preference ivabradin;

Antithrombotic agents are preferably understood to mean compounds from the group of the platelet aggregation inhibitors, the anticoagulants or the profibrinolytic substances.

In a preferred embodiment of the invention, the inventive compounds are administered in combination with a platelet aggregation inhibitor, by way of example and with preference aspirin, clopidogrel, prasugrel, ticagrelor, ticlopidin or dipyridamole.

In a preferred embodiment of the invention, the inventive compounds are administered in combination with a thrombin inhibitor, by way of example and with preference ximelagatran, dabigatran, melagatran, bivalirudin or clexane.

In a preferred embodiment of the invention, the inventive compounds are administered in combination with a GPIIb/IIIa antagonist such as, by way of example and with preference, tirofiban or abciximab.

In a preferred embodiment of the invention, the inventive compounds are administered in combination with a factor Xa inhibitor, by way of example and with preference rivaroxaban (BAY 59-7939), DU-176b, apixaban, betrixaban, otamixaban, fidexaban, razaxaban, letaxaban, eribaxaban, fondaparinux, idraparinux, PMD-3112, darexaban (YM-150), KFA-1982, EMD-503982, MCM-17, MLN-1021, DX 9065a, DPC 906, JTV 803, SSR-126512 or SSR-128428.

In a preferred embodiment of the invention, the inventive compounds are administered in combination with a factor XI or factor XIa inhibitor, by way of example and with preference FXI ASO-LICA, BAY 121-3790, MAA868, BMS986177, EP-7041 or AB-022.

In a preferred embodiment of the invention, the inventive compounds are administered in combination with heparin or with a low molecular weight (LMVV) heparin derivative.

In a preferred embodiment of the invention, the inventive compounds are administered in combination with a vitamin K antagonist, by way of example and with preference coumarin.

Hypotensive agents are preferably understood to mean compounds from the group of the calcium antagonists, angiotensin All antagonists, ACE inhibitors, endothelin antagonists, renin inhibitors, alpha-receptor blockers, beta-receptor blockers, mineralocorticoid receptor antagonists, rho-kinase inhibitors and the diuretics.

In a preferred embodiment of the invention, the inventive compounds are administered in combination with a calcium antagonist, by way of example and with preference nifedipine, amlodipine, verapamil or diltiazem.

In a preferred embodiment of the invention, the inventive compounds are administered in combination with an alpha-1-receptor blocker, by way of example and with preference prazosin.

In a preferred embodiment of the invention, the inventive compounds are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the inventive compounds are administered in combination with an angiotensin All antagonist, by way of example and with preference losartan, candesartan, valsartan, telmisartan or embusartan or a dual angiotensin All antagonist/neprilysin-inhibitor, by way of example and with preference LCZ696 (valsartan/sacubitril).

In a preferred embodiment of the invention, the inventive compounds are administered in combination with an ACE inhibitor, by way of example and with preference enalapril, captopril, lisinopril, ramipril, delapril, fosinopril, quinopril, perindopril or trandopril.

In a preferred embodiment of the invention, the inventive compounds are administered in combination with an endothelin antagonist, by way of example and with preference bosentan, darusentan, ambrisentan or sitaxsentan.

In a preferred embodiment of the invention, the inventive compounds are administered in combination with a renin inhibitor, by way of example and with preference aliskiren, SPP-600 or SPP-800.

In a preferred embodiment of the invention, the inventive compounds are administered in combination with a mineralocorticoid receptor antagonist, by way of example and with preference spironolactone or eplerenone.

In a preferred embodiment of the invention, the inventive compounds are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein(a) antagonists.

In a preferred embodiment of the invention, the inventive compounds are administered in combination with a CETP inhibitor, by way of example and with preference dalcetrapib, anacetrapib, torcetrapib (CP-529 414), JJT-705 or CETP vaccine (Avant).

In a preferred embodiment of the invention, the inventive compounds are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the inventive compounds are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the inventive compounds are administered in combination with a squalene synthesis inhibitor, by way of example and with preference BMS-188494 or TAK-475.

In a preferred embodiment of the invention, the inventive compounds are administered in combination with an ACAT inhibitor, by way of example and with preference avasimibe, melinamide, pactimibe, eflucimibe or SMP-797.

In a preferred embodiment of the invention, the inventive compounds are administered in combination with an MTP inhibitor, by way of example and with preference implitapide, BMS-201038, R-103757 or JTT-130.

In a preferred embodiment of the invention, the inventive compounds are administered in combination with a PPAR-gamma agonist, by way of example and with preference pioglitazone or rosiglitazone.

In a preferred embodiment of the invention, the inventive compounds are administered in combination with a PPAR-delta agonist, by way of example and with preference GW 501516 or BAY 68-5042.

In a preferred embodiment of the invention, the inventive compounds are administered in combination with a cholesterol absorption inhibitor, by way of example and with preference ezetimibe, tiqueside or pamaqueside.

In a preferred embodiment of the invention, the inventive compounds are administered in combination with a lipase inhibitor, a preferred example being orlistat.

In a preferred embodiment of the invention, the inventive compounds are administered in combination with a polymeric bile acid adsorbent, by way of example and with preference cholestyramine, colestipol, colesolvam, CholestaGel or colestimide.

In a preferred embodiment of the invention, the inventive compounds are administered in combination with a bile acid reabsorption inhibitor, by way of example and with preference ASBT(=IBAT) inhibitors, for example AZD-7806, S-8921, AK-105, BARI-1741, SC-435 or SC-635.

In a preferred embodiment of the invention, the inventive compounds are administered in combination with a lipoprotein(a) antagonist, by way of example and with preference, gemcabene calcium (CI-1027) or nicotinic acid.

In a preferred embodiment of the invention, the inventive compounds are administered in combination with a lipoprotein(a) antagonist, by way of example and with preference, gemcabene calcium (CI-1027) or nicotinic acid.

In a preferred embodiment of the invention, the inventive compounds are administered in combination with sGC modulators, by way of example and with preference, riociguat, cinaciguat or vericiguat.

In a preferred embodiment of the invention, the inventive compounds are administered in combination with an agent affecting the glucose metabolism, by way of example and with preference, insuline, a sulfonyl urea, acarbose, DPP4 inhibitors, GLP-1 analogs or SGLT-1 inhibitors.

In a preferred embodiment of the invention, the compounds according to the invention are administered in combination with a TGFbeta antagonist, by way of example and with preference pirfenidone or fresolimumab.

In a preferred embodiment of the invention, the compounds according to the invention are administered in combination with a CCR2 antagonist, by way of example and with preference CCX-140.

In a preferred embodiment of the invention, the compounds according to the invention are administered in combination with a TNFalpha antagonist, by way of example and with preference adalimumab.

In a preferred embodiment of the invention, the compounds according to the invention are administered in combination with a galectin-3 inhibitor, by way of example and with preference GCS-100.

In a preferred embodiment of the invention, the compounds according to the invention are administered in combination with a Nrf-2 inhibitor, by way of example and with preference bardoxolone

In a preferred embodiment of the invention, the compounds according to the invention are administered in combination with a BMP-7 agonist, by way of example and with preference THR-184.

In a preferred embodiment of the invention, the compounds according to the invention are administered in combination with a NOX1/4 inhibitor, by way of example and with preference GKT-137831.

In a preferred embodiment of the invention, the compounds according to the invention are administered in combination with a medicament which affects the vitamin D metabolism, by way of example and with preference calcitriol, alfacalcidol, doxercalciferol, maxacalcitol, paricalcitol, cholecalciferol or paracalcitol.

In a preferred embodiment of the invention, the compounds according to the invention are administered in combination with a cytostatic agent, by way of example and with preference cyclophosphamide.

In a preferred embodiment of the invention, the compounds according to the invention are administered in combination with an immunosuppressive agent, by way of example and with preference ciclosporin.

In a preferred embodiment of the invention, the compounds according to the invention are administered in combination with a phosphate binder, by way of example and with preference colestilan, sevelamer hydrochloride and sevelamer carbonate, Lanthanum and lanthanum carbonate.

In a preferred embodiment of the invention, the compounds according to the invention are administered in combination with renal proximal tubule sodium-phosphate co-transporter, by way of example and with preference, niacin or nicotinamide.

In a preferred embodiment of the invention, the compounds according to the invention are administered in combination with a calcimimetic for therapy of hyperparathyroidism.

In a preferred embodiment of the invention, the compounds according to the invention are administered in combination with agents for iron deficit therapy, by way of example and with preference iron products.

In a preferred embodiment of the invention, the compounds according to the invention are administered in combination with agents for the therapy of hyperurikaemia, by way of example and with preference allopurinol or rasburicase.

In a preferred embodiment of the invention, the compounds according to the invention are administered in combination with glycoprotein hormone for the therapy of anaemia, by way of example and with preference erythropoietin.

In a preferred embodiment of the invention, the compounds according to the invention are administered in combination with biologics for immune therapy, by way of example and with preference abatacept, rituximab, eculizumab or belimumab.

In a preferred embodiment of the invention, the compounds according to the invention are administered in combination with vasopressin antagonists (group of the vaptanes) for the treatment of heart failure, by way of example and with preference tolvaptan, conivaptan, lixivaptan, mozavaptan, satavaptan or relcovaptan.

In a preferred embodiment of the invention, the compounds according to the invention are administered in combination with Jak inhibitors, by way of example and with preference ruxolitinib, tofacitinib, baricitinib, CYT387, GSK2586184, lestaurtinib, pacritinib (SB1518) or TG101348.

In a preferred embodiment of the invention, the compounds according to the invention are administered in combination with prostacyclin analogs for therapy of microthrombi.

In a preferred embodiment of the invention, the compounds according to the invention are administered in combination with an alkali therapy, by way of example and with preference sodium bicarbonate.

In a preferred embodiment of the invention, the compounds according to the invention are administered in combination with an mTOR inhibitor, by way of example and with preference everolimus or rapamycin.

In a preferred embodiment of the invention, the compounds according to the invention are administered in combination with an NHE3 inhibitor, by way of example and with preference AZD1722 or tenapanor.

In a preferred embodiment of the invention, the compounds according to the invention are administered in combination with an eNOS modulator, by way of example and with preference sapropterin.

In a preferred embodiment of the invention, the compounds according to the invention are administered in combination with a CTGF inhibitor, by way of example and with preference FG-3019.

The total amount of the active ingredient to be administered will generally range from about 0.001 mg/kg to about 200 mg/kg body weight per day, and preferably from about 0.01 mg/kg to about 50 mg/kg body weight per day, and more preferably from about 0.01 mg/kg to about 10 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Nevertheless, it may optionally be necessary to deviate from the stated amounts, namely depending on body weight, route of administration, individual response to the active substance, type of preparation and time point or interval when application takes place. Thus, in some cases it may be sufficient to use less than the aforementioned minimum amount, whereas in other cases the stated upper limit must be exceeded. When applying larger amounts, it may be advisable to distribute these in several individual doses throughout the day.

According to a further embodiment, the compounds of formula (I) according to the invention are administered orally once or twice or three times a day. According to a further embodiment, the compounds of formula (I) according to the invention are administered orally once or twice a day. According to a further embodiment, the compounds of formula (I) according to the invention are administered orally once a day. For the oral administration, a rapid release or a modified release dosage form may be used.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

### EXPERIMENTAL SECTION

Abbreviations and Acronyms:
aq. aqueous (solution)
Boc tert.-butoxycarbonyl
br. broad (signal in NMR)
CDI 1,1′-carbonyldiimidazole
d day(s); doublet (in NMR)
DCI direct chemical ionization (in MS)
DCM dichloromethane
dd doublet of doublets (in NMR)

DIPEA N, N-diisopropylethylamine
DMAP 4-N,N-dimethylaminopyridine
DMF N,N-dimethylformamide
DMSO dimethylsulfoxide
dppp 1,3-bis(diphenylphosphino)propane
EDC N-ethyl-N′-(3-dimethylaminopropyl)carbodiimide hydrochloride
eq equivalent(s)
ESI electrospray ionization (in MS)
h hour(s)
HATU O-(7-azabenzotriazol-1-yl)-N,N,N′,N′-tetramethyluronium-hexafluorophosphate
HOAt 1-hydroxy-7-azabenzotriazole
HOBt 1-hydroxy-1H-benzotriazole hydrate
HPLC high-pressure/high-performance liquid chromatography
HV high vacuum
LC/MS liquid chromatography-coupled mass spectrometry
m multiplet (in NMR)
min minute(s)
MS mass spectrometry
MTBE methyl tert.-butyl ether
NMP N-methylpyrrolidone
NMR nuclear magnetic resonance spectrometry
PTSA p-toluenesulfonic acid
q quartet or quadruplet (in NMR)
quant. quantitative (yield)
quin quintet (in NMR)
RP reverse phase (in HPLC)
RT or rt room temperature
Rₜ retention time (in HPLC, LC/MS)
s singlet (in NMR)
sext sextet (in NMR)
SFC supercritical fluid chromatography
t triplet (in NMR)
TFA trifluoroacetic acid
THF tetrahydrofuran
TLC thin-layer chromatography
T3P® propylphosphonic anhydride (2,4,6-tripropyl-1,3,5,2,4,6-trioxatriphosphinane 2,4,6-trioxide)

Other abbreviations not specified herein have their meanings customary to the skilled person.

The various aspects of the invention described in this application are illustrated by the following examples which are not meant to limit the invention in any way.

The example testing experiments described herein serve to illustrate the present invention and the invention is not limited to the examples given.

### EXPERIMENTAL SECTION—GENERAL PART

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol, or from Separtis such as Isolute® Flash silica gel or Isolute® Flash NH2 silica gel in combination with a Isolera autopurifier (Biotage) and eluents such as gradients of e.g. hexane/EE or dichloromethane/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

NMR peak forms are stated as they appear in the spectra, possible higher order effects have not been considered.

The ¹H-NMR data of selected compounds are listed in the form of ¹H-NMR peaklists. For each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of target compounds (also the subject of the invention), and/or peaks of impurities. The peaks of stereoisomers, and/or peaks of impurities are typically displayed with a lower intensity compared to the peaks of the target compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the target compounds by known methods (MestReC, ACD simulation, or by use of empirically evaluated expectation values), can isolate the peaks of target compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures). In the peak picking routine, as described in the Research Disclosure Database Number 605005, the parameter “MinimumHeight” can be adjusted between 1% and 4%. Depending on the chemical structure and/or depending on the concentration of the measured compound it may be reasonable to set the parameter “MinimumHeight” <1%.

IUPAC names of the following intermediates and example compounds were generated using the ACD/Name software (batch version 14.00; Advanced Chemistry Development, Inc.) or the naming tool implemented in the BIOVIA Draw software (version 4.2 SP1; Dassault Systemes SE).

Reactions employing microwave irradiation may be run with a Biotage Initator® microwave oven optionally equipped with a robotic unit. The reported reaction times employing microwave heating are intended to be understood as fixed reaction times after reaching the indicated reaction temperature.

In order that this invention may be better understood, the following examples are set forth. These examples are for the purpose of illustration only, and are not to be construed as limiting the scope of the invention in any manner. All publications mentioned herein are incorporated by reference in their entirety.

Analytical HPLC, LC/MS and GC/MS Methods

Method 1:

Column: Kinetex EVO-C18 (Phenomenex), 2.6 μm, 3.0×50 mm; mobile phase A: 5 mM ammonium formate in water, mobile phase B: acetonitrile; gradient: 0.0 min 10% B→1.2 min 95% B→2.0 min 95% B; column temperature: 40° C.; flow rate: 1.3 ml/min.

Method 2:

Column: Ascentis Express C18 (Supelco), 2.7 μm, 2.1×50 mm; mobile phase A: 0.05% TFA in water, mobile phase B: 0.05% TFA in acetonitrile; gradient: 0.0 min 5% B→2.0 min 95% B→3.0 min 95% B; column oven: 40° C.; flow rate: 1.0 ml/min.

Method 3:

MS instrument: Thermo Scientific FT-MS; instrument UHPLC+: Thermo Scientific UltiMate 3000; column: Waters HSS T3, 2.1×75 mm, C18 1.8 μm; eluent A: 1 L water+0.01% formic acid, eluent B: 1 L acetonitrile +0.01% formic acid; gradient: 0.0 min 10% B→2.5 min 95% B→3.5 min 95% B; oven: 50° C.; flow rate: 0.90 ml/min; UV detection: 210 nm/optimum integration path 210-300 nm.

Method 4:

MS instrument: Waters Single Quad MS System; Waters UPLC Acquity; column: Waters BEH C18, 1.7 μm, 50×2.1 mm; eluent A: 1 L water+1.0 ml aq. ammonium hydroxide solution (25% ammonia), eluent B: 1 L acetonitrile; gradient: 0.0 min 92% A→0.1 min 92% A→1.8 min 5% A→3.5 min 5% A; column oven: 50° C.; flow rate: 0.45 ml/min; UV detection: 210 nm (208-400 nm).

Method 5:

Column: Ascentis Express C18 (Supelco), 2.7 μm, 2.1×50 mm; mobile phase A: 0.05% TFA in water, mobile phase B: 0.05% TFA in acetonitrile; gradient: 0.0 min 5% B→1.2 min 100% B→2.0 min 100% B; column oven: 40° C.; flow rate: 1.0 ml/min.

Method 6:

Column: CORTECS C18 (Waters), 2.7 μm, 2.1×50 mm; mobile phase A: 0.1% formic acid in water, mobile phase B: 0.1% formic acid in acetonitrile; gradient: 0.0 min 5% B→1.2 min 100% B→2.0 min 100% B; column oven: 40° C.; flow rate: 1.0 ml/min.

Method 7:

Instrument: Waters Acquity SQD UPLC System; column: Waters Acquity UPLC HSS T3 1.8 μm, 50×1 mm; eluent A: 1 L water+0.25 ml formic acid, eluent B: 1 L acetonitrile+0.25 ml formic acid; gradient: 0.0 min 90% A→1.2 min 5% A→2.0 min 5% A; column oven: 50° C.; flow rate: 0.40 ml/min; UV detection: 208-400 nm.

Method 8:

Column: Shim-pack XR-ODS (Shimadzu), 2.2 μm, 3.0×50 mm; mobile phase A: 0.05% TFA in water, mobile phase B: 0.05% TFA in acetonitrile; gradient: 0.0 min 5% B→1.2 min 100% B→2.6 min 100% B; column oven: 40° C.; flow rate: 1.0 ml/min.

Method 9:

Column: Ascentis Express C18 (Supelco), 2.7 μm, 3.0×50 mm; mobile phase A: 0.05% TFA in water, mobile phase B: 0.05% TFA in acetonitrile; gradient: 0.0 min 5% B→2.0 min 95% B→3.0 min 95% B; column oven: 40° C.; flow rate: 1.0 ml/min.

Method 10:

Column: Ascentis Express C18 (Supelco), 2.7 μm, 2.1×50 mm; mobile phase A: 0.05% TFA in water, mobile phase B: 0.05% TFA in acetonitrile; gradient: 0.0 min 5% B→1.1 min 100% B→2.0 min 100% B; column oven: 40° C.; flow rate: 1.0 ml/min.

Method 11:

Instrument: Waters Acquity SQD UPLC System; column: Waters Acquity UPLC HSS T3 1.8 μm, 50×1 mm; eluent A: 1 L water+0.25 ml formic acid, eluent B: 1 L acetonitrile+0.25 ml formic acid; gradient: 0.0 min 95% A→6.0 min 5% A→7.5 min 5% A; column oven: 50° C.; flow rate: 0.35 ml/min; UV detection: 210-400 nm.

Method 12:

Column: Ascentis Express C18 (Supelco), 2.7 μm, 3.0×50 mm; mobile phase A: 0.05% TFA in water, mobile phase B: 0.05% TFA in acetonitrile; gradient: 0.0 min 5% B→1.2 min 95% B→2.0 min 95% B; column oven: 40° C.; flow rate: 1.5 ml/min.

Method 13:

Column: Shim-pack XR-ODS (Shimadzu), 2.2 μm, 3.0×50 mm; mobile phase A: 0.05% TFA in water, mobile phase B: 0.05% TFA in acetonitrile; gradient: 0.0 min 5% B→1.2 min 100% B→2.0 min 100% B; column oven: 40° C.; flow rate: 1.2 ml/min.

Method 14:

Column: Kinetex EVO-C18 (Phenomenex), 2.6 μm, 3.0×50 mm; mobile phase A: 0.1% formic acid in water, mobile phase B: 0.1% formic acid in acetonitrile; gradient: 0.0 min 10% B→1.1 min 100% B→2.0 min 100% B; column oven: 40° C.; flow rate: 1.5 ml/min.

Method 15:

Column: Kinetex EVO-C18 (Phenomenex), 2.6 μm, 4.6×50 mm; mobile phase A: 5 mM ammonium carbonate in water, mobile phase B: acetonitrile; gradient: 0.0 min 10% B→1.45 min 95% B→2.0 min 95% B; column oven: 40° C.; flow rate: 1.8 ml/min.

Method 16:

Column: Kinetex EVO-C18 (Phenomenex), 2.6 μm, 2.1×50 mm; mobile phase A: 5 mM ammonium carbonate in water, mobile phase B: acetonitrile; gradient: 0.0 min 10% B→2.0 min 95% B→3.0 min 95% B; column oven: 40° C.; flow rate: 1.2 ml/min.

Method 17:

Column: Ascentis Express C18 (Supelco), 2.7 μm, 2.1×50 mm; mobile phase A: 0.05% TFA in water, mobile phase B: 0.05% TFA in acetonitrile; gradient: 0.0 min 5% B→4.0 min 95% B→5.0 min 95% B; column oven: 40° C.; flow rate: 1.0 ml/min.

Method 18:

Column: Ascentis Express C18 (Supelco), 2.7 pm, 2.1×50 mm; mobile phase A: 0.05% TFA in water, mobile phase B: 0.05% TFA in acetonitrile; gradient: 0.0 min 5% B→1.2 min 100% B→2.0 min 100% B; column oven: 40° C.; flow rate: 1.5 ml/min.

Method 19:

Column: Kinetex EVO-C18 (Phenomenex), 2.6 pm, 3.0×50 mm; mobile phase A: 0.1% formic acid in water, mobile phase B: 0.1% formic acid in acetonitrile; gradient: 0.0 min 10% B→3.5 min 95% B→5.0 min 95% B; column oven: 40° C.; flow rate: 1.5 ml/min.

Method 20:

Column: Ascentis Express C18 (Supelco), 2.7 pm, 3.0×50 mm; mobile phase A: 0.1% formic acid in water, mobile phase B: 0.1% formic acid in acetonitrile; gradient: 0.0 min 10% B→1.1 min 100% B→2.0 min 100% B; column oven: 40° C.; flow rate: 1.5 ml/min.

Method 21:

Column: CORTECS C18 (Waters), 2.7 μm, 2.1×50 mm; mobile phase A: 0.1% formic acid in water, mobile phase B: 0.1% formic acid in acetonitrile; gradient: 0.0 min 10% B→2.1 min 95% B→3.0 min 95% B; column oven: 40° C.; flow rate: 1.5 ml/min.

Method 22:

Column: XBridge Shield RP18 (Waters), 3.5 μm, 4.6×50 mm; mobile phase A: 5 mM ammonium carbonate in water, mobile phase B: acetonitrile; gradient: 0.0 min 10% B→2.2 min 95% B→3.6 min 95% B; column oven: 40° C.; flow rate: 1.5 ml/min.

Method 23:

Column: CORTECS C18 (Waters), 2.7 μm, 2.1×50 mm; mobile phase A: 0.1% formic acid in water, mobile phase B: 0.1% formic acid in acetonitrile; gradient: 0.0 min 10% B→1.1 min 100% B→2.0 min 100% B; column oven: 40° C.; flow rate: 1.0 ml/min.

Method 24:

Column: Ascentis Express C18 (Supelco), 2.7 μm, 2.1×50 mm; mobile phase A: 0.05% TFA in water, mobile phase B: 0.05% TFA in acetonitrile; gradient: 0.0 min 5% B→3.5 min 50% B→4.2 min 95% B→5.0 min 95% B; column oven: 40° C.; flow rate: 1.0 ml/min.

Method 25:

Column: Ascentis Express C18 (Supelco), 2.7 μm, 3.0×50 mm; mobile phase A: 0.05% TFA in water, mobile phase B: 0.05% TFA in acetonitrile; gradient: 0.0 min 5% B→1.1 min 100% B→2.0 min 100% B; column oven: 40° C.; flow rate: 1.5 ml/min.

Method 26:

Column: Shim-pack XR-ODS (Shimadzu), 2.2 μm, 3.0×50 mm; mobile phase A: 0.05% TFA in water, mobile phase B: 0.05% TFA in acetonitrile; gradient: 0.0 min 5% B→2.2 min 100% B→3.6 min 100% B; column oven: 40° C.; flow rate: 1.0 ml/min.

Method 27:

Column: Waters Acquity UPLC HSS T3 1.8 μm, 50×2.1 mm; mobile phase A: 1 L water+0.25 ml formic acid, mobile phase B: 1 L acetonitrile+0.25 ml formic acid; gradient: 0.0 min 90% A→0.3 min 90% A→1.7 min 5% A→3.0 min 5% A; column oven: 50° C.; flow rate: 1.20 ml/min; UV detection: 205-305 nm.

Method 28:

Column: Ascentis Express C18 (Supelco), 2.7 μm, 3.0×50 mm; mobile phase A: 0.05% TFA in water, mobile phase B: 0.05% TFA in acetonitrile; gradient: 0.0 min 5% B→1.3 min 95% B→2.6 min 95% B; column oven: 40° C.; flow rate: 1.0 ml/min.

Method 29:

Column: Kinetex XB-C18 (Phenomenex), 2.6 μm, 3.0×50 mm; mobile phase A: 0.05% TFA in water, mobile phase B: 0.05% TFA in acetonitrile; gradient: 0.0 min 5% B→1.1 min 100% B→2.0 min 100% B; column oven: 45° C.; flow rate: 1.5 ml/min.

Method 30:

Column: CORTECS C18 (Waters), 2.7 μm, 2.1×50 mm; mobile phase A: 0.1% formic acid in water, mobile phase B: 0.1% formic acid in acetonitrile; gradient: 0.0 min 5% B→2.0 min 95% B→3.0 min 95% B; column oven: 40° C.; flow rate: 1.0 ml/min.

Method 31:

Column: Zorbax SB-Aq (Agilent), 50×2.1 mm, 1.8 μm; eluent A: water+0.025% formic acid, eluent B: acetonitrile (ULC)+0.025% formic acid; gradient: 0.0 min 98% A→0.9 min 25% A→1.0 min 5% A→1.4 min 5% A→1.41 min 98% A→1.5 min 98% A; column oven: 40° C.; flow rate: 0.60 ml/min; UV detection: DAD, 210 nm.

Method 32:

Column: Ascentis Express C18 (Supelco), 2.7 μm, 3.0×50 mm; mobile phase A: 0.05% TFA in water, mobile phase B: 0.05% TFA in acetonitrile; gradient: 0.0 min 5% B→1.7 min 95% B 3.0 min 95% B; column oven: 40° C.; flow rate: 1.5 ml/min.

Method 33:

Instrument: Waters Acquity UPLC MS SingleQuad; column: Acquity UPLC BEH C18 1.7 μm, 50×2.1 mm; eluent A: water+0.2% aq. ammonia (32%), eluent B: acetonitrile; gradient: 0-1.6 min 1-99% B, 1.6-2.0 min 99% B; flow rate: 0.8 ml/min; column oven: 60° C.; DAD scan: 210-400 nm.

Method 34:

Instrument: Waters Acquity UPLC MS SingleQuad; column: Acquity UPLC BEH C18 1.7 μm, 50×2.1 mm; eluent A: water+0.1% formic acid (99%), eluent B: acetonitrile; gradient: 0-1.6 min 1-99% B, 1.6-2.0 min 99% B; flow rate: 0.8 ml/min; column oven: 60° C.; DAD scan: 210-400 nm.

Method 35:

Instrument: Thermo Scientific DSQII, Thermo Scientific Trace GC Ultra; column: Restek RTX-35MS, 15 m×200 μm×0.33 μm; constant flow of helium: 1.20 ml/min; oven: 60° C.; inlet: 220° C.; gradient: 60° C., 30° C./min→300° C. (3.33 min hold).

Method 36:

Instrument: Agilent 1290 UPLC MS 6230 TOF; column: BEH C18 1.7 μm, 50×2.1 mm; eluent A: water+0.05% formic acid (99%), eluent B: acetonitrile+0.05% formic acid (99%); gradient: 0-1.7 min 2-90% B, 1.7-2.0 min 90% B; flow rate: 1.2 ml/min; column oven: 60° C.; DAD scan: 190-400 nm.

Method 37:

Column: XBridge Shield RP18 (Waters), 3.5 μm, 4.6×50 mm; mobile phase A: 5 mM ammonium bicarbonate in water, mobile phase B: methanol; gradient: 0.0 min 5% B→7.0 min 95% B→10.0 min 95% B; column oven: 40° C.; flow rate: 1.2 ml/min.

Method 38:

Column: Ascentis Express C18 (Supelco), 2.7 μm, 2.1×50 mm; mobile phase A: 0.05% TFA in water, mobile phase B: 0.05% TFA in acetonitrile; gradient: 0.0 min 5% B→3.0 min 40% B→4.0 min 95% B→5.0 min 95% B; column oven: 40° C.; flow rate: 1.0 ml/min.

Method 39:

Column: Ascentis Express C18 (Supelco), 2.7 μm, 2.1×50 mm; mobile phase A: 0.1% of formic acid in water, mobile phase B: 0.1% of formic acid in acetonitrile; gradient: 0.0 min 10% B→2.1 min 95% B→3.0 min 95% B; column oven: 45° C.; flow rate:1.0 ml/min.

Method 40:

Column: Kinetex XB-C18 (Phenomenex), 2.6 μm, 3.0×50 mm; mobile phase A: 0.1% of formic acid in water, mobile phase B: 0.1% of formic acid in acetonitrile; gradient: 0.0 min 5% B→1.1 min 100% B→1.7 min 100% B; column oven: 40° C.; flow rate: 1.2 ml/min.

Method 41:

Column: Ascentis Express C18 (Supelco), 2.7 μm, 3.0×50 mm; mobile phase A: 0.05% TFA in water, mobile phase B: 0.05% TFA in acetonitrile; gradient: 0.0 min 5% B→2.0 min 95% B→2.7 min 95% B; column oven: 40° C.; flow rate: 1.5 ml/min.

Method 42:

Column: CORTECS C18 (Waters), 2.7 μm, 2.1×50 mm; mobile phase A: 0.1% of formic acid in water, mobile phase B: 0.1% of formic acid in acetonitrile; gradient: 0.0 min 5% B→1.1 min 100% B→1.6 min 100% B; column oven: 40° C.; flow rate: 0.8 ml/min.

Method 43:

Column: CORTECS C18 (Waters), 2.7 μm, 2.1×50 mm, mobile phase A: 0.1% of formic acid in water, mobile phase B: 0.1% of formic acid in acetonitrile; gradient: 0.0 min 5% B→2.0 min 95% B→2.6 min 95% B; column oven: 40° C.; flow rate: 0.8 ml/min.

Method 44:

Column: CORTECS C18 (Waters), 2.7 μm, 2.1×50 mm; mobile phase A: 0.05% TFA in water, mobile phase B: 0.05% TFA in acetonitrile; gradient: 0.0 min 5% B→1.1 min 100% B→2.0 min 100% B; column oven: 40° C.; flow rate: 1.0 ml/min.

Method 45:

Column: Omega, 3.0 μm, 2.1×30 mm; mobile phase A: 0.1% of formic acid in water, mobile phase B: 0.1% of formic acid in acetonitrile; gradient: 0.0 min 5% B→2.0 min 95% B→2.6 min 95% B; column oven: 40° C.; flow rate: 1.2 ml/min.

Method 46:

Column: Ascentis Express C18 (Supelco), 2.7 μm, 3.0×50 mm; mobile phase A: 0.05% TFA in water, mobile phase B: 0.05% TFA in acetonitrile; gradient: 0.0 min 5% B→2.1 min 100% B→2.8 min 100% B; column oven: 40° C.; flow rate: 1.5 ml/min.

Method 47:

Column: Ascentis Express C18 (Supelco), 2.7 μm, 3.0×50 mm; mobile phase A: 0.05% TFA in water, mobile phase B: 0.05% TFA in acetonitrile; gradient: 0.0 min 5% B→3.0 min 100% B→4.5 min 95% B; column oven: 40° C.; flow rate: 1.5 ml/min.

Method 48:

Column: Kinetex EVO-C18 (Phenomenex), 2.6 μm, 3.0×50 mm; mobile phase A: 5 mM ammonium hydrogen carbonate in water, mobile phase B: acetonitrile; gradient: 0.0 min 10% B→2.0 min 95% B→2.6 min 95% B; column oven 40° C.; flow rate: 1.2 ml/min.

Method 49:

Column: CORTECS C18 (Waters), 2.7 μm, 2.1×50 mm; mobile phase A: 0.1% of formic acid in water, mobile phase B: 0.1% of formic acid in acetonitrile; gradient: 0.0 min 5% B→2.0 min 95% B→2.6 min 95% B; column oven: 40° C.; flow rate: 1.2 ml/min.

Method 50:

Column: Shim-pack XR-ODS (Shimadzu), 2.2 μm, 3.0×50 mm; mobile phase A: 0.05% trifluoroacetic acid in water, mobile phase B: 0.05% trifluoroacetic acid in acetonitrile; gradient: 0.0 min 5% B→2.0 min 95% B→2.7 min 95% B; column oven: 40° C.; flow rate: 1.5 ml/min.

Method 51:

Column: CORTECS C18 (Waters), 2.7 μm, 2.1×50 mm; mobile phase A: 0.1% formic acid in water, mobile phase B: 0.1% formic acid in acetonitrile; gradient: 0.0 min 30% B→3.5 min 95% B→4.1 min 95% B; column oven: 40° C.; flow rate: 0.8 ml/min.

Method 52:

Column: Ascentis Express C18 (Supelco), 2.7 μm, 2.1×50 mm; mobile phase A: 0.05% trifluoroacetic acid in water, mobile phase B: 0.05% trifluoroacetic acid in acetonitrile; gradient: 0.0 min 5% B→2.0 min 95% B→2.7 min 95% B; column oven: 40° C.; flow rate: 1.5 ml/min.

Method 53:

Instrument: Waters MS SQ Detektor2, GC Agilent A7890; column: Restek RTX-35MS, 15 m×200 μm×0.33 μm; constant flow of helium: 1.20 ml/min; oven: 60° C.; inlet: 240° C.; gradient: 60° C., 30° C./min→300° C. (3.33 min hold).

Preparative HPLC Methods:

Method P1:

Column: Chromatorex C-18, 125×30 mm; eluent A: water+0.1% formic acid, eluent B: acetonitrile; gradient: 90:10→5:95; flow rate: 75 ml/min; UV detection: 210 nm.

Method P2:

Column: Chromatorex C-18, 125×30 mm; eluent A: water+0.1% TFA, eluent B: acetonitrile; gradient: 90:10→5:95; flow rate: 75 ml/min; UV detection: 210 nm.

Method P3:

Instrument: Waters Prep LC/MS System; column: XBridge C18 5 μm, 100×30 mm; eluent A: water, eluent B: acetonitrile; flow rate: 65 ml/min plus 5.0 ml of aq. ammonia (2% ammonia in water); at-column injection; gradient: 0.0-2.0 min 30% B, 2.0-2.2 min 30% B→50% B, 2.2-7.0 min 50% B→90% B, 7.0-7.5 min 90% B→92% B, 7.5-9.0 min 92% B; column oven: RT; UV detection: 200-400 nm.

Method P4:

Column: XBridge C18 5 μm, 75×30 mm; eluent A: water, eluent B: acetonitrile/water 80:20+1% aq. ammonium hydroxide solution, eluent C: acetonitrile; gradient: 0.0 min 95% A+5% B→1.0 min 95% A+5% B→6.5 min 67.6% A+5% B+27.4% C→6.84 min 5% B+95% C→7.85 min 5% B+95% C→8.12 min 95% A+5% B; flow rate: 80 ml/min; UV detection: 210 nm.

Method P5:

Column: Chromatorex C-18, 10 μm, 250×40 mm; eluent A: water+0.1% formic acid, eluent B: acetonitrile; gradient: 0-2.5 min 10% B, 2.5-14.5 min 10% B→95% B, 14.5-20 min 95% B; flow rate: 150 ml/min; UV detection: 210 nm.

Method P6:

Instrument: Waters Prep LC/MS System; column: XBridge C18 5 μm, 100×30 mm; eluent A: water, eluent B: acetonitrile; flow rate: 65 ml/min plus 5.0 ml of aq. ammonia (2% ammonia in water); at-column injection; gradient: 0.0-2.0 min 50% B, 2.0-2.2 min 50% B→70% B, 2.2-7.0 min 70% B→92% B, 7.0-9.0 min 92% B; column oven: RT; UV detection: 200-400 nm.

Method P7:

Instrument: Waters Prep LC/MS System; column: XBridge C18 5 μm, 100×30 mm; eluent A: water, eluent B: acetonitrile; flow rate: 65 ml/min plus 5.0 ml of aq. ammonia (2% ammonia in water); at-column injection; gradient: 0.0-2.0 min 10% B, 2.0-2.2 min 10% B→30% B, 2.2-7.0 min 30% B→70% B, 7.0-7.5 min 70%→92% B, 7.5-9.0 min 92% B; column oven: RT; UV detection: 200-400 nm.

Method P8:

Instrument: Waters Prep LC/MS System; column: XBridge C18 5 μm, 100×30 mm; eluent A: water, eluent B: acetonitrile; flow rate: 65 ml/min plus 5.0 ml of aq. ammonia (2% ammonia in water); at-column injection; gradient: 0.0-2.0 min 30% B, 2.0-2.2 min 30% B→50% B, 2.2-7.0 min 50% B→90% B, 7.0-7.5 min 90% B→92% B, 7.5-9.0 min 92% B; column oven: RT; UV detection: 200-400 nm.

Method P9:

Instrument: Waters Prep LC/MS System; column: XBridge C18 5 μm, 100×30 mm; eluent A: water, eluent B: acetonitrile; flow rate: 65 ml/min plus 5.0 ml of aq. ammonia (2% ammonia in to water); at-column injection; gradient: 0.0-2.0 min 10% B, 2.0-2.2 min 10% B→20% B, 2.2-7.0 min 20% B→60% B, 7.0-7.5 min 60% B→92% B, 7.5-9.0 min 92% B; column oven: RT; UV detection: 200-400 nm.

Method P10:

Instrument: Waters Prep LC/MS System; column: Phenomenex Kinetex C18 5 μm, 100×30 mm; eluent A: water, eluent B: acetonitrile; flow rate: 65 ml/min plus 5.0 ml of 2% aq. formic acid solution; at-column injection; gradient: 0.0-2.0 min 10% B, 2.0-2.2 min 10% B→30% B, 2.2-7.0 min 30% B→70% B, 7.0-7.5 min 70% B→92% B, 7.5-9.0 min 92% B; column oven: RT; UV detection: 200-400 nm.

Method P11:

Column: Chromatorex C-18, 125×30 mm; eluent A: water+0.1% formic acid, eluent B: acetonitrile; gradient: 0.0-2.5 min 10% B, 2.5-14.5 min 10% B→95% B, 14.5-20.0 min 95% B; flow rate: 75 ml/min; UV detection: 210 nm.

Method P12:

Column: Chromatorex RP C-18 10 μm, 125×30 mm; eluent A: water+0.1% aq. ammonia, eluent B: acetonitrile; manual injection: Rheodyne 3725i038; column oven: RT; UV detection: 208-400 nm.

Method P13:

Column: Chromatorex RP C-18 10 μm, 125×30 mm; eluent A: water, eluent B: 1 L acetonitrile+5 ml formic acid (2% in water); manual injection: Rheodyne 3725i038; column oven: RT; UV detection: 208-400 nm.

Method P14:

Column: Reprosil C-18, 205×50 mm; eluent A: water+0.1% of formic acid; eluent B: acetonitrile; gradient: 0.0-5.0 min 10% B, 5.0-17.5 min 10% B to 95% B, 17.5-21.0 min 95% B; flow rate: 150 ml/min, UV-Detection: 210 nm.

Method P15:

Column: Phenomenex Gemini C18 250×50 mm×10 μm; eluent A: water+0.05% of ammonia; eluent B: acetonitrile; gradient: 0-28 min 10% B→35% B.

Method P16:

Column: Chromatorex C18, 10 μm, 125 mm x 30 mm; eluent A: water+0.1% formic acid, eluent B: acetonitrile. Gradient: 0.00-4.88 min 80% A; 4.88-20.03 min gradient from 80% to 5% A; 20.03-23.00 min gradient from 5% to 80% A; 23.00-24.99 min 80% A; column temperature: room temperature; flow rate: 75 mL/min; UV detection: 210 nm.

Preparative Chromatography on a Chiral Stationary Phase

The racemic compound was dissolved in an appropriate solvent and submitted to preparative chromatography on a chiral stationary phase. The stationary phases were commercially available. A suitable column for enantiomer separation was selected from the following Daicel Phases based on analytical test runs: AD-H, AS-H, AY-H, AZ-H, OJ-H, OD-H, OZ-H, OX-H, IA, IB, IC, ID, IE, IF, IG, IH. The enantiomers were either separated by liquid chromatorgraphy or supercritical is fluid chromatography. For liquid chromatography, mixtures of n-heptane and ethanol or n-heptane and 2-propanol were used as the mobile phase, to which diethylamine or triethylamine were added as basic modifiers or trifluoroacetic acid or formic acid were added as acidic modifiers, respectively, as appropriate. For supercritical fluid chromatography, the same stationary phases were employed, using mixtures of supercritical carbondioxide with either methanol, ethanol or 2-propanol as the mobile phase. When appropriate, diethylamine was added to the alcoholic solvent as a basic modifier. In general, the enantiomer eluting first is called “enantiomer 1”, while the enantiomer eluting second is called “enantiomer 2”.

Starting Compounds and Intermediates

### Intermediate 1A

### Diethyl 1-[2-(naphthalen-2-yl)-2-oxoethyl]-1H-pyrazole-3,5-dicarboxylate

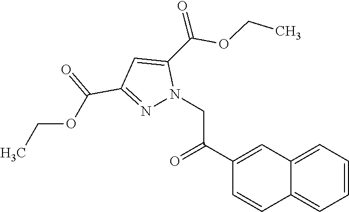 [SMILES: CCOC(=O)c1cc(C(=O)OCC)n(CC(=O)c2ccc3ccccc3c2)n1]

To a solution of diethyl 1H-pyrazole-3,5-dicarboxylate (5.00 g, 23.6 mmol) in acetone (60 ml) were added 2-bromo-1-(2-naphthyl)ethanone (5.87 g, 23.6 mmol) and potassium carbonate (4.23 g, 30.6 mmol). The mixture was stirred at RT overnight. After filtering off the solids, the filtrate was concentrated under reduced pressure, diluted with water and extracted with ethyl acetate. The combined organic layers were washed with brine, dried over anhydrous sodium sulfate and filtered. The filtrate was evaporated under reduced pressure to give 8.60 g (93% of theory, 97% purity) of the title compound.

LC/MS [Method 1]: Rₜ=2.13 min; MS (ESIpos): m/z=381 [M+H]⁺.

¹H-NMR (300 MHz, CDCl₃): δ [ppm]=8.53 (s, 1H), 7.92-8.04 (m, 4H), 7.59-7.70 (m, 2H), 7.51 (s, 1H), 6.28 (s, 2H), 4.46 (q, 2H), 4.31 (q, 2H), 1.44 (t, 3H), 1.34 (t, 3H).

### Intermediate 2A

### Ethyl 6-(2-naphthyl)-4-oxo-4,5-dihydropyrazolo[1,5-a]pyrazine-2-carboxylate

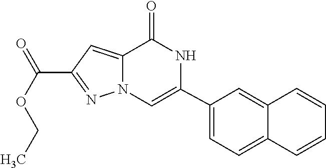 [SMILES: CCOC(=O)c1cc2c(=O)[nH]c(-c3ccc4ccccc4c3)cn2n1]

To a solution of diethyl 1-[2-(2-naphthyl)-2-oxoethyl]-1H-pyrazole-3,5-dicarboxylate (Intermediate 1A, 2.00 g, 5.3 mmol) in acetic acid (20 ml) was added ammonium acetate (10.10 g, 131.4 mmol). After refluxing overnight, the reaction mixture was cooled to RT and concentrated under reduced pressure. The residue was diluted with water and neutralized with sodium hydroxide. The precipitate was collected by filtration, washed with water and dried in vacuo to give 1.50 g (81% of theory, 92% purity) of the title compound.

LC/MS [Method 1]: Rₜ=1.80 min; MS (ESIpos): m/z=334 [M+H]⁺.

¹H-NMR (300 MHz, DMSO-d₆): δ [ppm]=8.47 (s, 1H), 8.34 (s, 1H), 7.94-8.05 (m, 4H), 7.55-7.60 (m, 2H), 7.27 (s, 1H), 4.35 (q, 2H), 1.35 (t, 3H).

### Intermediate 3A

### 6-(2-Naphthyl)-4-oxo-4,5-dihydropyrazolo[1,5-a]pyrazine-2-carboxylic acid

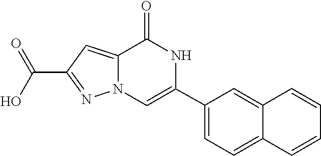 [SMILES: O=C(O)c1cc2c(=O)[nH]c(-c3ccc4ccccc4c3)cn2n1]

To a suspension of ethyl 6-(2-naphthyl)-4-oxo-4,5-dihydropyrazolo[1,5-a]pyrazine-2-carboxylate (Intermediate 2A, 25.00 g, 75.0 mmol) in water (500 ml) and ethanol (500 ml) was added 2 M sodium hydroxide solution (300 ml, 600.0 mmol). After stirring at RT for 2 h, the resulting mixture was diluted with water and then adjusted to pH 1-2 with concentrated hydrochloric acid under ice-bath cooling. The solid was collected by filtration and dried in vacuo to give 17.00 g (73% of theory, purity 98%) of the title compound.

LC/MS [Method 2]: Rₜ=1.17 min; MS (ESIpos): m/z=306 [M+H]⁺.

¹H-NMR (300 MHz, DMSO-d₆): δ [ppm]=13.30 (s, 1H), 11.90 (s, 1H), 8.40 (s, 1H), 8.33 (s, 1H), 7.98-8.06 (m, 3H), 7.88-7.92 (m, 1H), 7.59-7.63 (m, 2H), 7.40 (s, 1H).

### Intermediate 4A

### (1S)-1-Amino-2-methyl-1-phenylpropan-2-ol

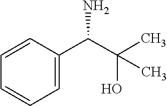

To a solution of methyl (2S)-amino(phenyl)ethanoate hydrochloride (500 mg, 2.48 mmol) in THF (10 ml) was added bromo(methyl)magnesium (18.0 ml, 1.0 M solution in THF, 18.0 mmol) dropwise at 0° C. After complete addition, the reaction mixture was warmed to RT and stirred overnight. The reaction was then quenched with 1.0 M hydrochloric acid, and the aqueous layer was washed with methyl tent.-butyl ether. The organic layer was discarded. The aqueous layer was basified with 1.0 M aqueous sodium hydroxide solution and extracted with ethyl acetate. The combined organic layers were dried over magnesium sulfate, and the volatiles were removed in vacuo. The residue was purified by column chromatography on basic silica gel (eluent: gradient of methanol in dichloromethane). Yield: 243 mg (59% of theory).

LC/MS [Method 4]: Rₜ=1.01 min; MS (ESIpos): m/z=166 [M+H]⁺.

¹H-NMR (400 MHz, DMSO-d₆): δ [ppm]=7.38-7.31 (m, 2H), 7.28-7.22 (m, 2H), 7.22-7.16 (m, 1H), 4.35 (s, 1H), 3.67 (s, 1H), 1.84 (br. s, 2H), 1.01 (s, 3H), 0.95 (s, 3H).

### Intermediate 5A

### tert.-Butyl 3-ethyl-3-hydroxyazetidine-1-carboxylate

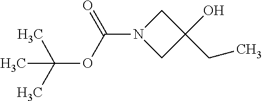

To a solution of tert.-butyl 3-oxoazetidine-1-carboxylate (50.00 g, 292.1 mmol) in THF (500 ml) was added ethyl magnesium bromide (350.5 ml, 350.5 mmol, 1 M in THF) dropwise with stirring at 0° C. under nitrogen atmosphere. After stirring at room temperature for 5 hours, the reaction mixture was cooled to 0° C., poured into 300 ml of aqueous ammonium chloride solution and extracted with ethyl acetate. The combined organic layers were washed with brine, dried over anhydrous sodium sulfate and filtered. The filtrate was evaporated under reduced pressure to give 58.00 g (98% of theory) of the title compound.

¹H-NMR (300 MHz, DMSO-d₆): δ [ppm]=5.48 (s, 1H), 3.55-3.66 (m, 4H), 1.60 (q, 2H), 1.38 (s, 9H), 0.85 (t, 3H).

### Intermediate 6A

### tert.-Butyl 3-ethyl-3-[(methylsulfonyl)oxy]azetidine-1-carboxylate

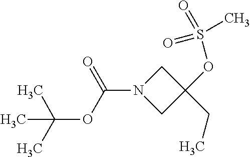 [SMILES: CCC1(OS(C)(=O)=O)CN(C(=O)OC(C)(C)C)C1]

To an ice-cold solution of tert.-butyl 3-ethyl-3-hydroxyazetidine-1-carboxylate (Intermediate 5A, 45.00 g, 223.6 mmol) and triethylamine (46.75 ml, 335.4 mmol) in dry dichloromethane (500 ml) was added dropwise methylsulfonyl chloride (30.73 g, 268.3 mmol). After stirring at room temperature overnight, the reaction mixture was diluted with water and extracted with dichloromethane. The combined organic layers were washed with water and brine, dried over anhydrous magnesium sulfate and filtered. The filtrate was evaporated under reduced pressure to give 70.00 g (95% of theory, 85% purity) of the title compound.

¹H-NMR (300 MHz, DMSO-d₆): δ [ppm]=4.09-4.12 (m, 2H), 3.87-3.90 (m, 2H), 3.28 (s, 3H), 2.02 (q, 2H), 1.37 (s, 9H), 0.94 (t, 3H).

### Intermediate 7A

### tert.-Butyl 3-azido-3-ethylazetidine-1-carboxylate

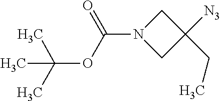 [SMILES: CCC1(N=[N+]=[N-])CN(C(=O)OC(C)(C)C)C1]

To a solution of tert.-butyl 3-ethyl-3-[(methylsulfonyl)oxy]azetidine-1-carboxylate (Intermediate 6A, 65.00 g, 197.8 mmol, 85% purity) in N,N-dimethylformamide (300 ml) was added sodium azide (25.72 g, 395.6 mmol) in portions. The resulting mixture was heated at 100° C. overnight. After cooling to room temperature, the reaction mixture was poured into 600 ml of water and extracted with ethyl acetate (3×600 ml). The combined organic layers were washed with brine, dried over anhydrous sodium sulfate and filtered. The filtrate was evaporated under reduced pressure to give 35.37 g (19% of theory, 25% purity) of the title compound.

¹H-NMR (300 MHz, DMSO-d₆): δ [ppm]=3.95-4.10 (m, 2H), 3.65-3.70 (m, 2H), 1.80 (q, 2H), 1.40 (s, 9H), 0.88 (t, 3H).

### Intermediate 8A

### tert.-Butyl 3-amino-3-ethylazetidine-1-carboxylate

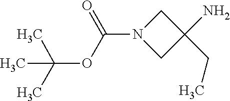

To a solution of tert.-butyl 3-azido-3-ethylazetidine-1-carboxylate (Intermediate 7A, 3.20 g, 3.5 mmol, 25% purity) in methanol (50 ml) was added 10% palladium on activated carbon (3.00 g). The resulting mixture was vigorously stirred overnight at ambient temperature under a hydrogen atmosphere (2-3 atm) and then filtered through Celite. The filtrate was evaporated under reduced pressure, and the residue was purified by flash-chromatography on silica gel (eluent: petroleum ether/ethyl acetate 4:1) to give 551.7 mg (77% of theory) of the title compound.

¹H-NMR (300 MHz, DMSO-d₆): δ [ppm]=3.48-3.59 (m, 4H), 2.00 (br. s, 2H), 1.54 (q, 2H), 1.38 (s, 9H), 0.85 (t, 3H).

### Intermediate 9A

### tert.-Butyl 3-hydroxy-3-(6-methoxypyridin-3-yl)azetidine-1-carboxylate

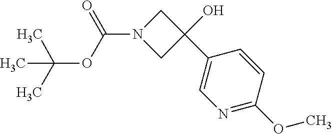 [SMILES: COc1ccc(C2(O)CN(C(=O)OC(C)(C)C)C2)cn1]

To a solution of 5-bromo-2-methoxypyridine (30.00 g, 159.6 mmol) in THF (500 ml) was added n-butyl lithium (70.2 ml, 175.5 mmol, 2.5 M in hexane) dropwise at −78° C. under nitrogen atmosphere. After stirring for 30 minutes at −78° C., a solution of tert.-butyl 3-oxoazetidine-1-carboxylate (24.83 g, 145.0 mmol) in THF (100 ml) was added dropwise at −78° C. The resulting mixture was stirred at −78° C. for another 2 hours, then quenched with ice-water and extracted with ethyl acetate. The combined organic layers were washed with water and brine, dried over anhydrous sodium sulfate and filtered. The filtrate was concentrated under reduced pressure, and the resin due was purified by flash-chromatography on silica gel (eluent: petroleum ether/ethyl acetate 3:1) to give 40.00 g (92% of theory, 94% purity) of the title compound.

LC/MS [Method 5]: Rₜ=0.90 min; MS (ESIpos): m/z=281 [M+H]⁺.

¹H-NMR (400 MHz, DMSO-d₆): δ [ppm]=8.27 (s, 1H), 7.76-7.79 (m, 1H), 6.83 (d, 1H), 6.39 (s, 1H), 4.00-4.08 (m, 4H), 3.85 (s, 3H), 1.39 (s, 9H).

### Intermediate 10A

### tert.-Butyl 3-azido-3-(6-methoxypyridin-3-yl)azetidine-1-carboxylate

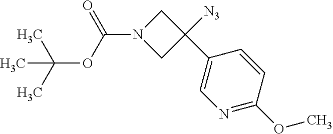 [SMILES: COc1ccc(C2(N=[N+]=[N-])CN(C(=O)OC(C)(C)C)C2)cn1]

Tert.-butyl 3-hydroxy-3-(6-methoxypyridin-3-yl)azetidine-1-carboxylate (Intermediate 9A, 20 g, 71.3 mmol) and triphenylphosphine (32.7 g, 124.9 mmol) were dissolved in 300 ml of THF under an atmosphere of nitrogen. Diphenyl phosphoroazidate (24.6 ml, 114.2 mmol) and diisopropyl azodicarboxylate (20.5 ml, 128.4 mmol) were added, and the reaction was stirred for 16 h at room temperature. The reaction was then quenched with water (250 ml) and extracted with ethyl acetate (3×200 ml). The combined extracts were washed with water (2×200 ml) and brine (2×200 ml) and dried over anhydrous sodium sulfate. Filtration, concentration and column chromatography on sillica gel (660 g silica gel; eluent: 0-30% ethyl acetate in petroleum ether) afforded the title compound. Yield: 7.4 g (32% of theory, 94% purity).

LC/MS [Method 6]: Rₜ=1.18 min; MS (ESIpos): m/z=306 [M+H]⁺.

¹H-NMR (300 MHz, DMSO-d₆): δ [ppm]=8.31 (s, 1H), 7.82 (d, 1H), 6.91 (d, 1H), 4.37 (d, 2H), 4.17 (d, 2H), 3.87 (s, 3H), 1.39 (s, 9H).

### Intermediate 11A

### tert.-Butyl 3-amino-3-(6-methoxypyridin-3-yl)azetidine-1-carboxylate

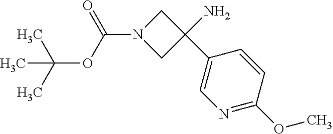 [SMILES: COc1ccc(C2(N)CN(C(=O)OC(C)(C)C)C2)cn1]

To a solution of tert.-butyl 3-azido-3-(6-methoxypyridin-3-yl)azetidine-1-carboxylate (Intermediate 10A, 5.00 g, 86% purity, 14.0 mmol) in methanol (100 ml) was added 10% palladium on active carbon (1.00 g, 940 μmol Pd), and the mixture was stirred under a hydrogen atmosphere (2-3 atm) at room temperature. After stirring overnight, the catalyst was filtered off, and the solvent was removed under reduced pressure. The residue was purified by flash-chromatography on silica gel (eluent: 0-70% ethyl acetate in petroleum ether) to give 2.50 g (60% of theory, 93% purity) of the title compound.

LC/MS [Method 1]: Rₜ=1.24 min; MS (ESIpos): m/z=280 [M+H]⁺.

¹H-NMR (300 MHz, DMSO-d₆): δ [ppm]=8.25 (s, 1H), 7.77 (d, 1H), 6.79 (d, 1H), 4.01 (d, 2H), 3.87 (d, 2H), 3.82 (s, 3H), 1.38 (s, 9H).

### Intermediate 12A

### tert.-Butyl (4S)-4-phenyl-1,2,3-oxathiazolidine-3-carboxylate 2,2-dioxide

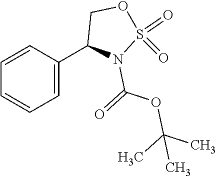 [SMILES: CC(C)(C)OC(=O)N1[C@@H](c2ccccc2)COS1(=O)=O]

Triethylamine (5.2 ml, 37 mmol) was added to a solution of thionylchloride (1.4 ml, 19 mmol) in dichloromethane (60.0 ml), and the mixture was cooled to −60° C. A solution of tert.-butyl [(1S)-2-hydroxy-1-phenylethyl]carbamate (4.00 g, 16.9 mmol) in dichloromethane (100 ml) was added dropwise, and the reaction was stirred at −60° C. for 2 h. After warming to RT, water was added and the layers were separated. The organic layer was washed with brine, dried over magnesium sulfate and evaporated to dryness. The residue was dissolved in acetonitrile (40 ml), and sodium periodate (3.97 g, 18.5 mmol) and ruthenium(III) chloride trihydrate (441 mg, 1.69 mmol) were added at 0° C. The mixture was stirred overnight at 0° C. and for 1 d at RT. Water and ethyl acetate were then added, and the insoluble material was removed by filtration. The layers were separated and the aqueous layer was extracted with ethyl acetate. The combined organic layers were washed with brine, dried over magnesium sulfate and evaporated to dryness to afford the title compound (4.20 g, 73% of theory, 88% purity). The product was used in the next step without further purification.

LC/MS [Method 3]: Rₜ=1.91 min; MS (ESIneg): m/z=344 [M−H+HCO₂H]⁻.

¹H-NMR (400 MHz, DMSO-d₆): δ [ppm]=7.48-7.28 (m, 5H), 5.53 (dd, 1H), 5.04 (dd, 1H), 4.61 (dd, 1H), 1.33 (s, 9H).

### Intermediate 13A

### tert.-Butyl[(1S)-2-(morpholin-4-yl)-1-phenylethyl]carbamate

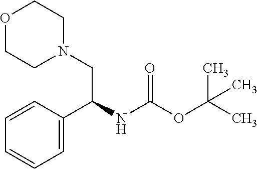 [SMILES: CC(C)(C)OC(=O)N[C@H](CN1CCOCC1)c1ccccc1]

A solution of tert.-butyl (4S)-4-phenyl-1,2,3-oxathiazolidine-3-carboxylate 2,2-dioxide (Intermediate 12A, 986 mg, 58% purity, 1.91 mmol) in THF (16.0 ml) was cooled to 0° C., and morpholine (670 μl, 7.6 mmol) was added. The reaction mixture was stirred at RT overnight before an aqueous solution of ammonium carbonate (1.0 M, 5.0 ml) was added. The mixture was again stirred at RT overnight before the pH was adjusted to 5, and stirring was continued at RT for 3 d. The reaction mixture was then evaporated to dryness to obtain the title compound. Yield: 660 mg (61% of theory, 54% purity). The product was used in the next step without further purification.

LC/MS [Method 3]: Rₜ=1.01 min; MS (ESIpos): m/z=307 [M+H]⁺.

### Intermediate 14A

### (1S)-2-(Morpholin-4-yl)-1-phenylethanamine dihydrochloride

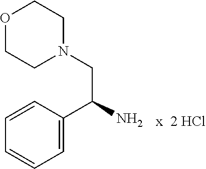 [SMILES: N[C@H](CN1CCOCC1)c1ccccc1]

A solution of tert.-butyl [(1S)-2-(morpholin-4-yl)-1-phenylethyl]carbamate (Intermediate 13A, 660 mg, 54% purity, 1.16 mmol) in dichloromethane (4.0 ml) was treated with anisole (630 μl, 5.8 mmol) and hydrogen chloride (2.9 ml, 4.0 M solution in dioxane, 12 mmol) at RT. After stirring at RT overnight, the reaction mixture was evaporated to dryness to afford the title compound. Yield: 453 mg (98% of theory, 70% purity). The product was used in the next step without further purification.

### Intermediate 15A

### Ethyl 4-methyl-3-{[(trifluoromethyl)sulfonyl]oxy}pent-2-enoate

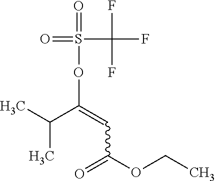 [SMILES: CCOC(=O)C=C(OS(=O)(=O)C(F)(F)F)C(C)C]

To a solution of ethyl 4-methyl-3-oxopentanoate 10.0 g (63.2 mmol) in toluene (400 ml) was added a solution of lithium hydroxide (11.4 g, 474.1 mmol) in water (120 ml) at 10° C. After stirring for 5 minutes at 10° C., trifluoromethanesulfonic anhydride (21.5 ml, 126.4 mmol) was added slowly to the reaction mixture maintaining the temperature below 25° C. After stirring for 2 h, the reaction mixture was poured into 400 ml of water and extracted with ethyl acetate. The combined organic layers were washed with water and brine and dried over anhydrous sodium sulfate. Filtration and evaporation gave 16.5 g (73% of theory, 81% purity) of the title compound.

LC/MS [Method 8]: Rₜ=1.43 min; MS (ESIpos): m/z=291 [M+H]⁺.

¹H-NMR (400 MHz, DMSO-d₆): δ [ppm]=6.12 (d, 1H), 4.17 (q, 2H), 2.57 (sept, 1H), 1.23 (t, 3H), 1.15 (d, 6H).

### Intermediate 16A

### Diethyl 4-isopropyl-1H-pyrazole-3,5-dicarboxylate

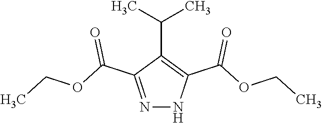 [SMILES: CCOC(=O)c1n[nH]c(C(=O)OCC)c1C(C)C]

To a solution of ethyl 4-methyl-3-[(trifluoromethyl)sulfonyl]pent-2-enoate (Intermediate 15A, 16.50 g, 48.9 mmol, purity 81%) in N,N-dimethylformamide (170 ml) were added ethyl 2-diazoacetate (8.45 g, 73.4 mmol), tetrakis(triphenylphosphine)palladium (2.83 g, 2.4 mmol) and 4-methylmorpholine (12.6 ml, 97.9 mmol) in sequence. The resulting mixture was stirred for 3 hours at room temperature and then heated overnight at 60° C. After cooling to room temperature, the reaction mixture was diluted with water (500 ml) and extracted with ethyl acetate. The combined organic layers were washed with water and brine, dried over anhydrous sodium sulfate and concentrated under reduced pressure. The residue was purified by silica gel column to chromatography (660 g silica gel; eluent: 20% ethyl acetate in petroleum ether) to give 7.66 g (61% of theory) of the title compound.

LC/MS [Method 9]: Rₜ=1.43 min; MS (ESIpos): m/z=255 [M+H]⁺.

¹H-NMR (300 MHz, DMSO-d₆): δ [ppm]=14.20 (s, 1H), 4.32 (br. s, 4H), 3.83-3.92 (m, 1H), 1.25-1.32 (m, 12H).

### Intermediate 17A

### Diethyl 1-[2-(naphthalen-2-yl)-2-oxoethyl]-4-(propan-2-yl)-1H-pyrazole-3,5-dicarboxylate

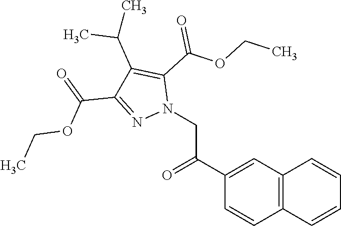 [SMILES: CCOC(=O)c1nn(CC(=O)c2ccc3ccccc3c2)c(C(=O)OCC)c1C(C)C]

Finely powdered potassium carbonate (982 mg, 7.11 mmol) and 2-bromo-1-(naphthalen-2-yl)-ethanone (1.64 g, 6.46 mmol) were added to a solution of diethyl 4-(propan-2-yl)-1H-pyrazole-3,5-dicarboxylate (Intermediate 16A, 1.66 g, 6.46 mmol) in acetone (28 ml). Three drops of water were added, and the mixture was stirred at RT overnight. Additional 2-bromo-1-(naphthalen-2-yl)ethanone (328 mg, 1.29 mmol) was added, and stirring was continued for 4 h at RT. The insoluble material was then filtered off and the filtrate was evaporated to dryness. The residue was dissolved in water and dichloromethane and the layers were separated. The aqueous layer was extracted with dichloromethane, and the combined organic layers were washed with brine, filtered over a hydrophobic phase separation filter paper and evaporated to dryness to afford the title compound. Yield: 3.50 g (59% of theory, 46% purity). The product was used in the next step without further purification.

LC/MS [Method 34]: Rₜ=1.47 min; MS (ESIpos): m/z=423 [M+H]⁺.

### Intermediate 18A

### Ethyl 6-(naphthalen-2-yl)-4-oxo-3-(propan-2-yl)-4,5-dihydropyrazolo[1,5-a]pyrazine-2-carboxylate

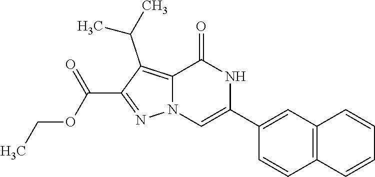 [SMILES: CCOC(=O)c1nn2cc(-c3ccc4ccccc4c3)[nH]c(=O)c2c1C(C)C]

A solution of diethyl 1-[2-(naphthalen-2-yl)-2-oxoethyl]-4-(propan-2-yl)-1H-pyrazole-3,5-dicarboxylate (Intermediate 17A, 3.50 g, 46% purity, 3.81 mmol) and ammonium acetate (5.87 g, 76.2 mmol) in acetic acid (34 ml) was heated under reflux overnight. After cooling to RT, the mixture was poured onto ice-water, and the mixture was neutralized by addition of aqueous sodium hydroxide solution. The precipitate was filtered off and washed with water. The collected solid was dissolved in dichloromethane, water was added, and the layers were separated. The aqueous layer was extracted with dichloromethane, and the combined organic layers were washed with brine, filtered over a hydrophobic phase separation filter paper and evaporated to dryness to afford the crude title compound. Yield: 2.48 g (quant). The product was used in the next step without further purification.

LC/MS [Method 34]: Rₜ=1.37 min; MS (ESIpos): m/z=376 [M+H]⁺.

### Intermediate 19A

### 6-(Naphthalen-2-yl)-4-oxo-3-(propan-2-yl)-4,5-dihydropyrazolo[1,5-a]pyrazine-2-carboxylic acid

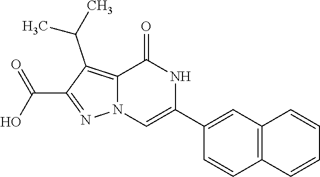 [SMILES: CC(C)c1c(C(=O)O)nn2cc(-c3ccc4ccccc4c3)[nH]c(=O)c12]

Aqueous sodium hydroxide solution (30.0 ml, 1.0 M, 30.0 mmol) was added to a suspension of ethyl 6-(naphthalen-2-yl)-4-oxo-3-(propan-2-yl)-4,5-dihydropyrazolo[1,5-a]pyrazine-2-carboxylate (Intermediate 18A, 1.40 g, 3.73 mmol) in ethanol (48 ml) and water (48 ml). The mixture was sonicated for 50 min and then stirred at RT overnight. The ethanol was distilled off, and the remaining aqueous phase was acidified with concentrated hydrochloric acid until pH 2. The precipitate was filtered off, washed with water and dried under reduced pressure at 100° C. to afford the title compound. Yield: 1.21 g (79% of theory, 84% purity). The product was used in the next step without further purification.

LC/MS [Method 34]: Rₜ=1.08 min; MS (ESIpos): m/z=348 [M+H]⁺.

### Intermediate 20A

### 2-Bromo-1-(3,4-dimethylphenyl)ethanone

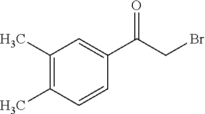

A solution of 1-(3,4-dimethylphenyl)ethanone (20.0 g, 135 mmol) and phenyltrimethylammonium tribromide (50.7 g, 135 mmol) in dichloromethane (200 ml) was stirred overnight at room temperature. The ammonium salts were filtered off, and the filter cake was washed with ethyl acetate. The filtrate was evaporated under reduced pressure to give 34.40 g (79% of theory, 70% purity) of the title compound, which was directly used for the next step without further purification.

¹H-NMR (400 MHz, CDCl₃): δ [ppm]=7.74 (s, 1H), 7.70 (d, 1H), 7.23 (d, 1H), 4.42 (s, 2H), 2.32 (s, 3H), 2.31 (s, 3H).

### Intermediate 21A

### Diethyl 1-[2-(3,4-dimethylphenyl)-2-oxoethyl]-1H-pyrazole-3,5-dicarboxylate

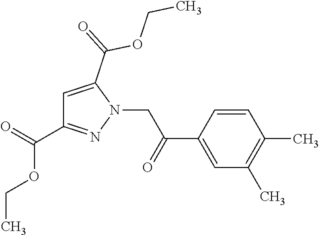 [SMILES: CCOC(=O)c1cc(C(=O)OCC)n(CC(=O)c2ccc(C)c(C)c2)n1]

A mixture of diethyl 1H-pyrazole-3,5-dicarboxylate (20.0 g, 94.3 mmol), 2-bromo-1-(3,4-dimethylphenyl)ethanone (Intermediate 20A, 33.6 g, 104 mmol, 70% purity) and potassium carbonate (14.3 g, 104 mmol) in acetone (250 ml) was stirred overnight at room temperature. The solids were filtered off, and the filtrate was concentrated and then partitioned between dichloromethane and water. The organic phase was washed with water and brine, dried over anhydrous sodium sulfate, filtered and evaporated under reduced pressure. The residue was purified by flash-chromatography on silica gel (eluent: petroleum ether/ethyl acetate 7:3) to give 35.0 g (91% of theory, 88% purity) of the title compound.

LC/MS [Method 10]: Rₜ=1.21 min; MS (ESIpos): m/z=359 [M+H]⁺.

¹H-NMR (400 MHz, CDCl₃): δ [ppm]=7.72 (s, 1H), 7.69 (d, 1H), 7.45 (s, 1H), 7.25 (d, 1H), 6.07 (s, 2H), 4.42 (q, 2H), 4.13 (q, 2H), 2.32 (s, 6H), 1.40 (t, 3H), 1.30 (t, 3H).

### Intermediate 22A

### Ethyl 6-(3,4-dimethylphenyl)-4-oxo-4,5-dihydropyrazolo[1,5-a]pyrazine-2-carboxylate

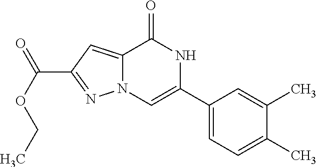 [SMILES: CCOC(=O)c1cc2c(=O)[nH]c(-c3ccc(C)c(C)c3)cn2n1]

To a solution of diethyl 1-[2-(3,4-dimethylphenyl)-2-oxoethyl]-1H-pyrazole-3,5-dicarboxylate (Intermediate 21A, 35.0 g, 86.3 mmol, 88% purity) in acetic acid (200 ml) was added ammonium acetate (133 g, 1.73 mol). The resulting mixture was heated to 110° C. overnight. After cooling to room temperature, the reaction mixture was poured into ice-water. The solid was collected by filtration, washed with water and dried in vacuo to give 25.0 g (93% of theory) of the title compound.

¹H-NMR (400 MHz, DMSO-d₆): δ [ppm]=11.68 (s, 1H), 8.13 (s, 1H), 7.57 (s, 1H), 7.48 (d, 1H), 7.40 (s, 1H), 7.25 (d, 1H), 4.34 (q, 2H), 2.29 (s, 3H), 2.27 (s, 3H), 1.33 (t, 3H).

### Intermediate 23A

### 6-(3,4-Dimethylphenyl)-4-oxo-4,5-dihydropyrazolo[1,5-a]pyrazine-2-carboxylic acid

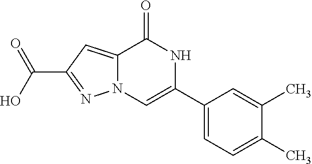 [SMILES: Cc1ccc(-c2cn3nc(C(=O)O)cc3c(=O)[nH]2)cc1C]

To a suspension of ethyl 6-(3,4-dimethylphenyl)-4-oxo-4,5-dihydropyrazolo[1,5-a]pyrazine-2-carboxylate (Intermediate 22A, 8.30 g, 26.7 mmol) in ethanol (40 ml) was added aqueous sodium hydroxide solution (20 ml, 3.0 M). After stirring for 4 h at RT, the pH value was adjusted to 6 with 3 M hydrochloric acid. The solid was collected by filtration, washed with water and dried in vacuo to give 6.00 g (76% of theory, 96% purity) of the title compound.

LC/MS [Method 2]: Rₜ=1.13 min; MS (ESIpos): m/z=284 [M+H]⁺.

¹H-NMR (400 MHz, DMSO-d₆): δ [ppm]=13.24 (s, 1H), 11.68 (s, 1H), 8.09 (s, 1H), 7.57 (s, 1H), 7.48 (d, 1H), 7.35 (s, 1H), 7.25 (d, 1H), 2.28 (s, 3H), 2.27 (s, 3H).

### Intermediate 24A

### Methyl 4-methyl-3,4-dihydro-2H-1,4-benzoxazine-6-carboxylate

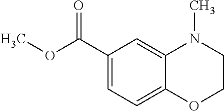

To a mixture of methyl 3,4-dihydro-2H-1,4-benzoxazine-6-carboxylate (5.00 g, 25.9 mmol) and potassium carbonate (3.93 g, 28.5 mmol) in N,N-dimethylformamide (75 ml) was added iodomethane (2.1 ml, 33.6 mmol) dropwise. After stirring at room temperature for 6 h, more iodomethane (2.1 ml, 33.6 mmol) was added. The reaction mixture was stirred overnight at room temperature. After filtering off the solids, the filtrate was diluted with water and extracted with ethyl acetate. The combined organic layers were concentrated under reduced pressure. The residue was purified by flash-chromatography on silica gel (eluent: petroleum ether/ethyl acetate 1:1) to give 2.60 g (46% of theory) of the title compound.

LC/MS [Method 12]: Rₜ=1.03 min; MS (ESIpos): m/z=208 [M+H]⁺.

¹H-NMR (300 MHz, DMSO-d₆): δ [ppm]=7.17-7.22 (m, 2H), 6.72 (d, 1H), 4.25-4.28 (m, 2H), 3.75 (s, 3H), 3.21-3.24 (m, 2H), 2.83 (s, 3H).

### Intermediate 25A

### 4-Methyl-3,4-dihydro-2H-1,4-benzoxazine-6-carboxylic acid

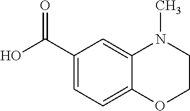

To a solution of methyl 4-methyl-3,4-dihydro-2H-1,4-benzoxazine-6-carboxylate (Intermediate 24A, 2.60 g, 12.5 mmol) in methanol (50 ml) and water (30 ml) was added sodium hydroxide (2.51 g, 62.7 mmol). After stirring at room temperature overnight, the reaction mixture was diluted with water (50 ml) and adjusted to pH 3 with 1.0 M hydrochloric acid. The precipitate was collected by filtration, washed with water and dried in vacuo to give 2.10 g (86% of theory, 94% purity) of the title compound.

LC/MS [Method 12]: Rₜ=0.81 min; MS (ESIpos): m/z=194 [M+H]⁺.

¹H-NMR (300 MHz, CD₃OD): δ [ppm]=7.33-7.36 (m, 2H), 6.72-6.76 (m, 1H), 4.33-4.36 (m, 2H), 3.27-3.30 (m, 2H), 2.93 (s, 3H).

### Intermediate 26A

### N-Methoxy-N,4-dimethyl-3,4-dihydro-2H-1,4-benzoxazine-6-carboxamide

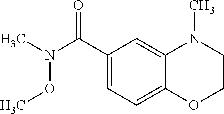 [SMILES: CON(C)C(=O)c1ccc2c(c1)N(C)CCO2]

To a solution of 4-methyl-3,4-dihydro-2H-1,4-benzoxazine-6-carboxylic acid (Intermediate 25A, 2.10 g, 10.9 mmol) in N,N-dimethylformamide (30 ml) were added 1-hydroxybenzotriazole (2.33 g, 15.2 mmol), 1-(3-dimethylaminopropyl)-3-ethylcarbodiimide hydrochloride (2.92 g, 15.2 mmol) and triethylamine (6.1 ml, 43.5 mmol). After stirring at room temperature for 5 min, N-methoxymethanamine hydrochloride (1.27 g, 13.0 mmol) was added. The reaction mixture was stirred overnight at room temperature, then poured into water and extracted with ethyl acetate. The combined organic layers were dried over anhydrous sodium sulfate and filtered. The filtrate was concentrated under reduced pressure. The residue was purified by flash-chromatography on silica gel (eluent: petroleum ether/ethyl acetate 3:2) to give 2.50 g (93% of theory, 96% purity) of the title compound.

LC/MS [Method 12]: Rₜ=0.83 min; MS (ESIpos): m/z=237 [M+H]⁺.

¹H-NMR (400 MHz, CDCl₃): δ [ppm]=7.06-7.15 (m, 2H), 6.74-6.78 (m, 1H), 4.33-4.36 (m, 2H), 3.62 (s, 3H), 3.35 (s, 3H), 3.27-3.30 (m, 2H), 2.92 (s, 3H).

### Intermediate 27A

### 1-(4-Methyl-3,4-dihydro-2H-1,4-benzoxazin-6-yl)ethanone

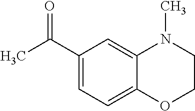

To a solution of N-methoxy-N,4-dimethyl-3,4-dihydro-2H-1,4-benzoxazine-6-carboxamide (Intermediate 26A, 2.50 g, 10.6 mmol) in THF (25 ml) was added methylmagnesium bromide (10.6 ml, 31.8 mmol, 3.0 M in diethyl ether) at −78° C. under a nitrogen atmosphere. The resulting solution was warmed to room temperature and stirred for 2 h at room temperature. The reaction mixture was then quenched with brine and extracted with ethyl acetate. The combined organic layers were washed with water and brine, dried over anhydrous sodium sulfate and filtered. The filtrate was evaporated under reduced pressure to give 1.80 g (82% of theory, 92% purity) of the title compound.

LC/MS [Method 13]: Rₜ=1.08 min; MS (ESIpos): m/z=192 [M+H]⁺.

¹H-NMR (300 MHz, CD₃OD): δ [ppm]=7.30-7.37 (m, 2H), 6.77 (d, 1H), 4.33-4.36 (m, 2H), 3.27-3.30 (m, 2H), 2.93 (s, 3H), 2.54 (s, 3H).

### Intermediate 28A

### 2-Bromo-1-(4-methyl-3,4-dihydro-2H-1,4-benzoxazin-6-yl)ethanone hydrobromide

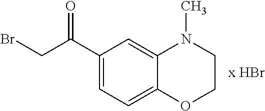

To a solution of 1-(4-methyl-3,4-dihydro-2H-1,4-benzoxazin-6-yl)ethanone (Intermediate 27A, 1.60 g, 8.4 mmol) in hydrogen bromide (20 ml, 33% in acetic acid) was added bromine (7.5 ml, 7.5 mmol, 1.0 M in acetic acid). The resulting mixture was stirred at 60° C. for 2 h. After cooling to room temperature, the reaction mixture was evaporated under reduced pressure to give 1.77 g (38% of theory, 63% purity) of the crude title compound, which was directly used for the next step without further purification.

LC/MS [Method 14]: Rₜ=1.11 min; MS (ESIpos): m/z=270/272 [M+H]⁺.

### Intermediate 29A

### Diethyl 1-[2-(4-methyl-3,4-dihydro-2H-1,4-benzoxazin-6-yl)-2-oxoethyl]-1H-pyrazole-3,5-dicarboxylate

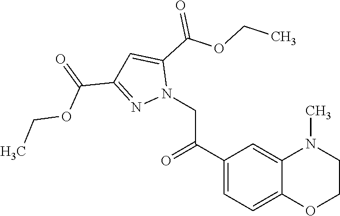 [SMILES: CCOC(=O)c1cc(C(=O)OCC)n(CC(=O)c2ccc3c(c2)N(C)CCO3)n1]

A mixture of 2-bromo-1-(4-methyl-3,4-dihydro-2H-1,4-benzoxazin-6-yl)ethanone hydrobromide (Intermediate 28A, 1.77 g, 3.2 mmol, 63% purity), diethyl 1H-pyrazole-3,5-dicarboxylate (642 mg, 3.0 mmol) and potassium carbonate (2.09 g, 15.1 mmol) in acetone (40 ml) was stirred overnight at room temperature. The solids were filtered off, and the filtrate was concentrated under reduced pressure. The residue was purified by flash-chromatography on silica gel (eluent: petroleum ether/ethyl acetate 2:1) to give 1.18 g (96% of theory) of the title compound.

LC/MS [Method 14]: Rₜ=1.21 min; MS (ESIpos): m/z=402 [M+H]⁺.

¹H-NMR (300 MHz, DMSO-d₆): δ [ppm]=7.44 (s, 1H), 7.28-7.33 (m, 2H), 6.83 (d, 1H), 6.06 (s, 2H), 4.36-4.45 (m, 4H), 4.28 (q, 2H), 3.28-3.31 (m, 2H), 2.92 (s, 3H), 1.40 (t, 3H), 1.31 (t, 3H).

### Intermediate 30A

### Ethyl 6-(4-methyl-3,4-dihydro-2H-1,4-benzoxazin-6-yl)-4-oxo-4,5-dihydropyrazolo[1,5-a]pyrazine-2-carboxylate

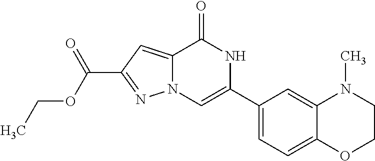 [SMILES: CCOC(=O)c1cc2c(=O)[nH]c(-c3ccc4c(c3)N(C)CCO4)cn2n1]

To a solution of diethyl 1-[2-(4-methyl-3,4-dihydro-2H-1,4-benzoxazin-6-yl)-2-oxoethyl]-1H-pyrazole-3,5-dicarboxylate (Intermediate 29A, 700 mg, 1.7 mmol) in acetic acid (20 ml) was added ammonium acetate (2.69 g, 34.9 mmol). The resulting mixture was stirred at 130° C. for 10 h under a nitrogen atmosphere. After cooling to room temperature, the mixture was poured into water. The solid was collected by filtration and then purified by flash-chromatography on silica gel (eluent: petroleum ether/ethyl acetate 1:8) to give 340 mg (49% of theory, 90% purity) of the title compound.

LC/MS [Method 15]: Rₜ=1.32 min; MS (ESIpos): m/z=355 [M+H]⁺.

¹H-NMR (300 MHz, CDCl₃): δ [ppm]=9.54 (br. s, 1H), 7.72 (s, 1H), 7.60 (s, 1H), 6.92-6.99 (m, 3H), 4.50 (q, 2H), 4.40-4.43 (m, 2H), 3.42-3.44 (m, 2H), 3.07 (s, 3H), 1.47 (t, 3H).

### Intermediate 31A

### 6-(4-Methyl-3,4-dihydro-2H-1,4-benzoxazin-6-yl)-4-oxo-4,5-dihydropyrazolo[1 ,5-a]pyrazine-2-carboxylic acid

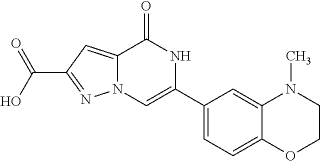 [SMILES: CN1CCOc2ccc(-c3cn4nc(C(=O)O)cc4c(=O)[nH]3)cc21]

To a solution of ethyl 6-(4-methyl-3,4-dihydro-2H-1,4-benzoxazin-6-yl)-4-oxo-4,5-dihydropyrazolo-[1,5-a]pyrazine-2-carboxylate (Intermediate 30A, 440 mg, 1.2 mmol) in ethanol (10 ml) was added a solution of sodium hydroxide (497 mg, 12.4 mmol) in water (3.0 ml). After stirring for 3 h at room temperature, the reaction mixture was concentrated and then diluted with water. The pH of the aqueous phase was adjusted to 3 with 1.0 M hydrochloric acid. The solid was collected by filtration, washed with water and dried in vacuo to give 343 mg (83% of theory) of the title compound.

LC/MS [Method 16]: Rₜ=1.27 min; MS (ESIpos): m/z=327 [M+H]⁺.

¹H-NMR (400 MHz, DMSO-d₆): δ [ppm]=11.62 (s, 1H), 8.07 (s, 1H), 7.33 (s, 1H), 7.05 (s, 1H), 6.97-7.01 (m, 1H), 6.75 (d, 1H), 4.26-4.29 (m, 2H), 3.26-3.29 (m, 2H), 2.93 (s, 3H).

### Intermediate 32A

### Diethyl 4-(trifluoromethyl)-1H-pyrazole-3,5-dicarboxylate

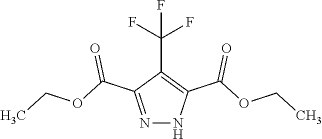 [SMILES: CCOC(=O)c1n[nH]c(C(=O)OCC)c1C(F)(F)F]

To a solution of ethyl 4,4,4-trifluorobut-2-ynoate (7.20 g, 43.3 mmol) in diethyl ether (80 ml) was added dropwise ethyl 2-diazoacetate (4.95 g, 4.56 ml, 15.7 mmol) at 0° C. within 15 minutes. The resulting mixture was warmed to room temperature and stirred overnight. The reaction mixture was then concentrated under reduced pressure. The residue was purified by flash-chromatography on silica gel (eluent: petroleum ether/ethyl acetate 5:1) to give 5.50 g (43% of theory, 95% purity) of the title compound.

¹H-NMR (300 MHz, DMSO-d₆): δ [ppm]=4.36 (q, 4H), 1.31 (t, 6H).

¹⁹F-NMR (282 MHz, DMSO-d₆): δ [ppm]=−53.62 (s, 3F).

### Intermediate 33A

### Diethyl 1-[2-(2-naphthyl)-2-oxoethyl]-4-(trifluoromethyl)-1H-pyrazole-3,5-dicarboxylate

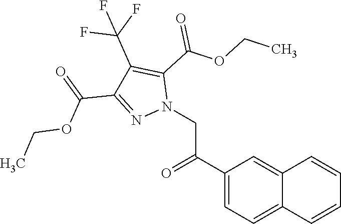 [SMILES: CCOC(=O)c1nn(CC(=O)c2ccc3ccccc3c2)c(C(=O)OCC)c1C(F)(F)F]

To a solution of 2-bromo-1-(2-naphthyl)ethanone (2.45 g, 7.9 mmol) in acetone (50 ml) were added diethyl 4-(trifluoromethyl)-1H-pyrazole-3,5-dicarboxylate (Intermediate 32A, 2.00 g, 7.1 mmol) and potassium carbonate (2.46 g, 4.5 mmol). The reaction mixture was stirred overnight at room temperature. The solids were then filtered off, and the filtrate was concentrated under reduced pressure. The residue was purified by flash-chromatography on silica gel (eluent: petroleum ether/ethyl acetate 9:1) to give 2.80 g (68% of theory, 78% purity) of the title compound.

LC/MS [Method 5]: Rₜ=1.33 min; MS (ESIpos): m/z=449 [M+H]⁺.

¹H-NMR (400 MHz, DMSO-d₆): δ [ppm]=8.82 (s, 1H), 8.00-8.18 (m, 4H), 7.67-7.76 (m, 2H), 6.42 (s, 2H), 4.37 (q, 2H), 4.26 (q, 2H), 1.31 (t, 3H), 1.11 (t, 3H).

### Intermediate 34A

### Ethyl 6-(2-naphthyl)-4-oxo-3-(trifluoromethyl)-4,5-dihydropyrazolo[1,5-a]pyrazine-2-carboxylate

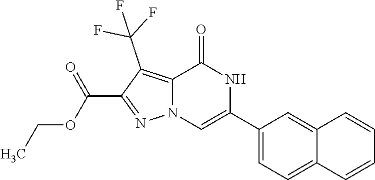 [SMILES: CCOC(=O)c1nn2cc(-c3ccc4ccccc4c3)[nH]c(=O)c2c1C(F)(F)F]

Ammonium acetate (7.50 g, 97.4 mmol) was added to a solution of diethyl 1-[2-(2-naphthyl)-2-oxoethyl]-4-(trifluoromethyl)-1H-pyrazole-3,5-dicarboxylate (Intermediate 33A, 2.80 g, 78% purity, 4.9 mmol) in acetic acid (30 ml), and the reaction mixture was stirred at 110° C. overnight. The solution was then poured into ice-water (50 ml), and the precipitate was filtered off, washed with water (2×50 ml) and dried to give 2.20 g (92% of theory, 82% purity) of the title compound.

LC/MS [Method 5]: Rₜ=1.20 min; MS (ESIpos): m/z=402 [M+H]⁺.

¹H-NMR (300 MHz, DMSO-d₆): δ [ppm]=12.26 (s, 1H), 8.41-8.43 (m, 2H), 7.98-8.07 (m, 3H), 7.87-7.91 (m, 1H), 7.60-7.64 (m, 2H), 4.41 (q, 2H), 1.34 (t, 3H).

### Intermediate 35A

### 6-(2-Naphthyl)-4-oxo-3-(trifluoromethyl)-4,5-dihydropyrazolo[1,5-a]pyrazine-2-carboxylic acid

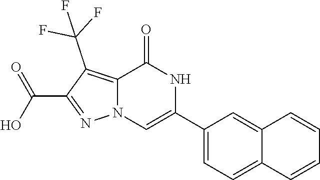 [SMILES: O=C(O)c1nn2cc(-c3ccc4ccccc4c3)[nH]c(=O)c2c1C(F)(F)F]

Sodium hydroxide (1.79 g, 44.9 mmol) was added to a solution of ethyl 6-(2-naphthyl)-4-oxo-3-(trifluoromethyl)-4,5-dihydropyrazolo[1,5-a]pyrazine-2-carboxylate (Intermediate 34A, 2.20 g, 82% purity, 4.5 mmol) in ethanol (45 ml) and water (5.0 ml), and the mixture was stirred at room temperature for 4 hours. The solution was then diluted with water (50 ml) and extracted with ethyl acetate (2×100 ml). The organic phases were discarded, and the aqueous layer was adjusted to pH 3 with 1 M hydrochloric acid. The solid was collected by filtration to afford 1.85 g (98% of theory, 89% purity) of the title compound.

LC/MS [Method 17]: Rₜ=1.79 min; MS (ESIpos): m/z=374 [M+H]⁺.

¹H-NMR (300 MHz, DMSO-d₆): δ [ppm]=14.07 (br. s, 1H), 12.22 (s, 1H), 8.38-8.41 (m, 2H), 7.98-8.07 (m, 3H), 7.87-7.91 (m, 1H), 7.60-7.63 (m, 2H).

### Intermediate 36A

### (1S)-1-Amino-1-(4-fluorophenyl)-2-methylpropan-2-ol

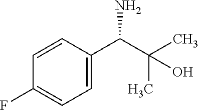

Methylmagnesium bromide (13 ml, 1.0 M solution in THF, 13 mmol) was added slowly to a solution of methyl (2S)-amino(4-fluorophenyl)ethanoate hydrochloride (485 mg, 2.21 mmol) in THF (9.7 ml). After complete addition, the reaction mixture was slowly warmed to RT and stirred at this temperature overnight. Hydrochloric acid solution (1.0 M) was then added, and the mixture was washed with MTBE. The layers were separated and the organic layer was discarded. The aqueous layer was brought to basic pH by addition of 1.0 M aqueous sodium hydroxide solution and was extracted with ethyl acetate. The combined organic layers were dried over magnesium sulfate, filtered and evaporated. The residue was purified by column chromatography on basic silica gel (eluent: gradient of methanol in dichloromethane). Yield: 203 mg (48% of theory, 95% purity).

LC/MS [Method 4]: Rₜ=1.08 min; MS (ESIpos): m/z=184 [M+H]⁺.

¹H-NMR (500 MHz, DMSO-d₆): δ [ppm]=7.44-7.30 (m, 2H), 7.13-7.03 (m, 2H), 4.36 (br. s, 1H), 3.69 (s, 1H), 2.07-1.75 (m, 2H), 1.00 (s, 3H), 0.94 (s, 3H).

### Intermediate 37A

### Methyl 4-methoxy-3-{[(trifluoromethyl)sulfonyl]oxy}but-2-enoate

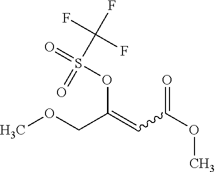

Triethylamine (21 ml, 150 mmol) was added to a solution of methyl 4-methoxy-3-oxobutanoate (20.0 g, 137 mmol) in dichloromethane (200 ml), and the mixture was cooled to −78° C. Trifluoro-methanesulfonic anhydride (25 ml, 150 mmol) was added dropwise at this temperature. After complete addition, the reaction mixture was warmed to RT and stirred for 3 h. The mixture was then diluted with dichloromethane (200 ml) and washed with water (2×300 ml) and brine (2×300 ml). The organic layer was dried over sodium sulfate, filtered and evaporated to afford the title compound. Yield: 36.0 g (85% of theory, purity 90%). The product was used in the next step without further purification.

¹H-NMR (400 MHz, DMSO-d₆): δ [ppm]=6.40-6.44 (m, 1H), 4.55 (s, 0.8H), 4.17 (s, 1.2H), 3.72-3.74 (m, 3H), 3.29-3.31 (m, 3H).

### Intermediate 38A

### 3-Ethyl 5-methyl 4-(methoxymethyl)-1H-pyrazole-3,5-dicarboxylate

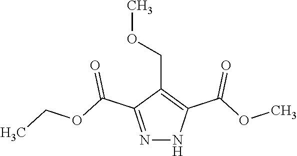 [SMILES: CCOC(=O)c1n[nH]c(C(=O)OC)c1COC]

A solution of methyl 4-methoxy-3-{[(trifluoromethyl)sulfonyl]oxy}but-2-enoate (Intermediate 37A, 37.0 g, 133 mmol), tetrakis(triphenylphosphine)palladium(0) (7.68 g, 6.7 mmol), 4-methylmorpholine (29.2 ml, 266 mmol) and ethyl diazoacetate (21.5 ml, 200 mmol) in N,N-dimethylformamide (400 ml) was stirred under an atmosphere of nitrogen at room temperature for 3 hours. Subsequently, the reaction mixture was heated to 60° C. and stirred overnight. After cooling to RT, the solution was poured into water (600 ml) and extracted with ethyl acetate (3×800 ml). The combined organic layers were washed with water (2×600 ml) and brine (2×600 ml) and dried over anhydrous sodium sulfate. Filtration, concentration and column chromatography on silica gel (660 g silica gel; eluent: petroleum ether/ethyl acetate 1:1) gave 14.35 g (41% of theory, 93% purity) of the title compound.

LC/MS [Method 14]: Rₜ=0.66 min; MS (ESIpos): m/z=243 [M+H]⁺.

¹H-NMR (300 MHz, DMSO-d₆): δ [ppm]=14.57 (s, 1H), 4.73 (s, 2H), 4.31 (q, 2H), 3.83-3.89 (m, 3H), 3.23 (s, 3H), 1.39 (t, 3H).

### Intermediate 39A

### Dimethyl 4-(methoxymethyl)-1H-pyrazole-3,5-dicarboxylate

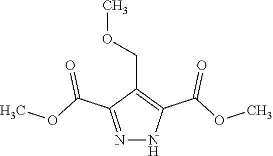

To a solution of 3-ethyl 5-methyl 4-(methoxymethyl)-1H-pyrazole-3,5-dicarboxylate (Intermediate 38A, 13.00 g, 51.0 mmol) in methanol (130 ml) was added sodium methoxide (10 ml, 15.3 mmol, 1.4 M solution in methanol). After stirring at 65° C. overnight, the reaction mixture was cooled to room temperature and quenched with sodium bicarbonate (2.00 g, 23.8 mmol). The solids were filtered off, and the filtrate was concentrated under reduced pressure. The residue was purified by flash-chromatography on silica gel (eluent: petroleum ether/ethyl acetate 3:1) to give 6.80 g (56% of theory, 96% purity) of the title compound.

LC/MS [Method 18]: Rₜ=0.62 min; MS (ESIpos): m/z=229 [M+H]⁺.

¹H-NMR (300 MHz, DMSO-d₆): δ [ppm]=14.62 (s, 1H), 4.73 (s, 2H), 3.85-3.87 (m, 6H), 3.22 (s, 3H).

### Intermediate 40A

### Dimethyl 4-(methoxymethyl)-1-[2-(2-naphthyl)-2-oxoethyl]-1H-pyrazole-3,5-dicarboxylate

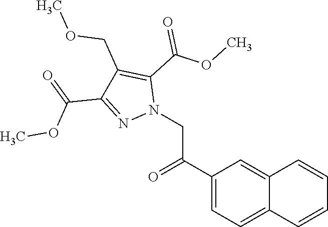 [SMILES: COCc1c(C(=O)OC)nn(CC(=O)c2ccc3ccccc3c2)c1C(=O)OC]

Dimethyl 4-(methoxymethyl)-1H-pyrazole-3,5-dicarboxylate (Intermediate 39A, 2.50 g, 10.7 mmol) and potassium carbonate (3.71 g, 26.8 mmol) were added to a solution of 2-bromo-1-(naphthalen-2-yl)ethanone (6.10 g, 53% purity, 12.9 mmol) in acetone (50 ml), and the mixture was stirred at RT overnight. The solids were then filtered off, and the filtrate was concentrated under reduced pressure. The residue was purified by column chromatography on silica gel (220 g silica gel, eluent: petroleum ether/ethyl acetate 3:1) to afford 1.80 g (33% of theory, 79% purity) of the title compound.

LC/MS [Method 12]: Rₜ=1.13 min; MS (ESIpos): m/z=419 [M+Na]⁺.

### Intermediate 41A

### Methyl 3-(methoxymethyl)-6-(2-naphthyl)-4-oxo-4,5-dihydropyrazolo[1,5-a]pyrazine-2-carboxylate

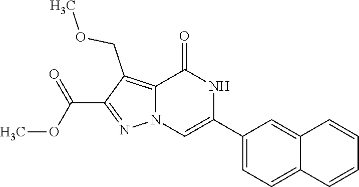 [SMILES: COCc1c(C(=O)OC)nn2cc(-c3ccc4ccccc4c3)[nH]c(=O)c12]

Ammonium acetate (5.53 g, 71.7 mmol) was added to a solution of dimethyl 4-(methoxymethyl)-1-[2-(naphthalen-2-yl)-2-oxoethyl]-1H-pyrazole-3,5-dicarboxylate (Intermediate 40A, 1.80 g, 79% purity, 3.59 mmol) in acetic acid (40.0 ml), and the mixture was heated to 110° C. for 18 h. After cooling to RT, the solution was diluted with water (100 ml). The precipitate was filtered off, washed with water (100 ml) and dried to afford the title compound. Yield: 1.20 g (63% of theory, 69% purity).

LC/MS [Method 12]: Rₜ=0.83 min; MS (ESIpos): m/z=364 [M+H]⁺.

¹H-NMR (300 MHz, DMSO-d₆): δ [ppm]=11.90 (s, 1H), 8.39 (s, 1H), 8.31 (s, 1H), 7.97-8.06 (m, 3H), 7.86-7.91 (m, 1H), 7.58-7.63 (m, 2H), 4.94 (s, 2H), 3.90 (s, 3H), 3.29 (s, 3H).

### Intermediate 42A

### 3-(Methoxymethyl)-6-(2-naphthyl)-4-oxo-4,5-dihydropyrazolo[1,5-a]pyrazine-2-carboxylic acid

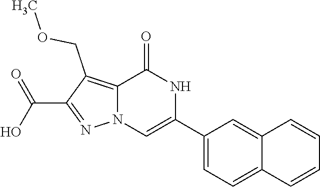 [SMILES: COCc1c(C(=O)O)nn2cc(-c3ccc4ccccc4c3)[nH]c(=O)c12]

A solution of sodium hydroxide (911.4 mg, 22.8 mmol) in water (15.0 ml) was added to a solution of methyl 3-(methoxymethyl)-6-(2-naphthyl)-4-oxo-4,5-dihydropyrazolo[1,5-a]pyrazine-2-carboxylate (Intermediate 41A, 1.20 g, 2.28 mmol) in methanol (15.0 ml), and the mixture was stirred at RT for 1 h. The solution was then concentrated under reduced pressure, and the residue was diluted with water (20 ml). The aqueous phase was washed with ethyl acetate (20 ml), and the organic layer was discarded. The aqueous layer was adjusted to pH 3 with 3.0 M hydrochloric acid. The precipitate was collected by filtration and dried to afford the title compound. Yield: 850 mg (93% of theory, 87% purity).

LC/MS [Method 19]: Rₜ=2.47 min; MS (ESIpos): m/z=350 [M+H]⁺.

¹H-NMR (400 MHz, DMSO-d₆): δ [ppm]=11.84 (s, 1H), 8.39 (s, 1H), 8.30 (s, 1H), 7.80-8.05 (m, 4H), 7.58-7.61 (m, 2H), 4.95 (s, 2H), 3.27 (s, 3H).

### Intermediate 43A

### tert.-Butyl 3-(4-fluorophenyl)-3-hydroxyazetidine-1-carboxylate

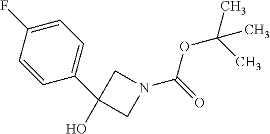 [SMILES: CC(C)(C)OC(=O)N1CC(O)(c2ccc(F)cc2)C1]

To a solution of tert.-butyl 3-oxoazetidine-1-carboxylate (10.0 g, 58.4 mmol) in THF (200 ml) was added (4-fluorophenyl)magnesium bromide (70 ml, 70.0 mmol, 1.0 M solution in THF) with stirring at 0° C. under an atmosphere of nitrogen. After stirring for 3 hours at room temperature, the reaction mixture was poured into a saturated aqueous solution of ammonium chloride. After extracting with dichloromethane, the combined organic layers were dried over anhydrous sodium sulfate and filtered. The filtrate was concentrated under reduced pressure, and the residue was purified by flash-chromatography on silica gel (eluent: petroleum ether/ethyl acetate 3:1) to give 10.2 g (73% of theory, 92% purity) of the title compound.

LC/MS [Method 5]: Rₜ=1.07 min; MS (ESIpos): m/z=212 [M+H−C₄H₈]⁺.

¹H-NMR (400 MHz, CDCl₃): δ [ppm]=7.46-7.50 (m, 2H), 7.05-7.10 (m, 2H), 4.22 (d, 2H), 4.16 (d, 2H), 1.46 (s, 9H).

Intermediate 44A

### tert.-Butyl 3-chloro-3-(4-fluorophenyl)azetidine-1-carboxylate

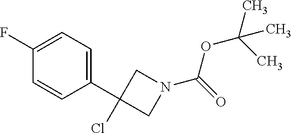 [SMILES: CC(C)(C)OC(=O)N1CC(Cl)(c2ccc(F)cc2)C1]

To a solution of tert.-butyl 3-(4-fluorophenyl)-3-hydroxyazetidine-1-carboxylate (Intermediate 43A, 10.60 g, 39.8 mmol) in dichloromethane (160 ml) were added triethylamine (8.3 ml, 59.7 mmol) and methanesulfonyl chloride (3.7 ml, 47.7 mmol) dropwise at 0° C. under a nitrogen atmosphere. The reaction mixture was stirred at RT for 24 h, then poured into 200 ml of water and extracted with dichloromethane. The combined organic layers were dried over anhydrous sodium sulfate and filtered. The filtrate was evaporated under reduced pressure, and the residue was purified by flash-chromatography on silica gel (eluent: petroleum ether/ethyl acetate 4:1) to give 7.80 g (69% of theory) of the title compound.

¹H-NMR (300 MHz, DMSO-d₆): δ [ppm]=7.54-7.59 (m, 2H), 7.24-7.30 (m, 2H), 4.64 (d, 2H), 4.40 (d, 2H), 1.40 (s, 9H).

### Intermediate 45A

### tert.-Butyl 3-azido-3-(4-fluorophenyl)azetidine-1-carboxylate

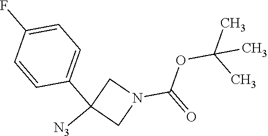 [SMILES: CC(C)(C)OC(=O)N1CC(N=[N+]=[N-])(c2ccc(F)cc2)C1]

To a solution of tent.-butyl 3-chloro-3-(4-fluorophenyl)azetidine-1-carboxylate (Intermediate 44A, 7.20 g, 25.2 mmol) in N,N-dimethylformamide (100 ml) was added sodium azide (8.19 g, 126.0 mmol) in portions. The resulting mixture was heated to 100° C. overnight. After cooling to room temperature, the reaction mixture was poured into 100 ml of water and extracted with ethyl acetate. The combined organic layers were washed with water and brine, dried over anhydrous sodium sulfate and filtered. The filtrate was concentrated under reduced pressure, and the residue was purified by flash-chromatography on silica gel (eluent: petroleum ether/ethyl acetate 4:1) to give 6.30 g (85% of theory) of the title compound.

¹H-NMR (300 MHz, CDCl₃): δ [ppm]=7.36-7.41 (m, 2H), 7.12-7.18 (m, 2H), 4.27-4.36 (m, 4H), 1.49 (s, 9H).

### Intermediate 46A

### 3-(4-Fluorophenyl)-1-methylazetidin-3-amine

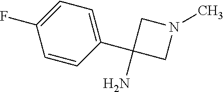

To a solution of tent.-butyl 3-azido-3-(4-fluorophenyl)azetidine-1-carboxylate (Intermediate 45A, 8.40 g, 25.9 mmol, 90% purity) in THF (200 ml) was added lithium aluminium hydride (3.93 g, 103.5 mmol) in portions at 0° C. The resulting mixture was stirred for 30 min at room temperature and then heated to 55° C. for 40 min. After cooling to room temperature, the reaction mixture was quenched with sodium sulfate decahydrate (30.0 g). The solids were filtered off and washed with methanol. The combined filtrate was concentrated under reduced pressure. The residue was purified by flash-chromatography on silica gel (eluent: dichloromethane/methanol 19:1 containing 0.05% triethylamine) to give 2.14 g (40% of theory, 88% purity) of the title compound.

¹H-NMR (300 MHz, DMSO-d₆): δ [ppm]=7.57-7.65 (m, 2H), 7.09-7.15 (m, 2H), 3.49-3.52 (m, 2H), 3.11-3.14 (m, 2H), 2.29 (s, 3H).

### Intermediate 47A

### (S)—N-[(1E)-1-(6-Methoxypyridin-3-yl)ethylidene]-2-methylpropane-2-sulfinamide

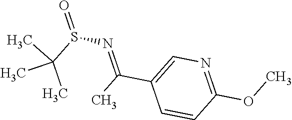 [SMILES: COc1ccc(/C(C)=N/[S@@](=O)C(C)(C)C)cn1]

To a solution of 1-(6-methoxypyridin-3-yl)ethanone (1.25 g, 97% purity, 8.0 mmol) in 1,2-dichloroethane (12.0 ml) were added (S)-2-methylpropane-2-sulfinamide (1.14 g, 9.9 mmol) and titanium(IV) ethoxide (6.7 ml, 32 mmol). The reaction mixture was heated to 110° C. for 2 h in a microwave reactor. For work-up, this reaction mixture was combined with a second reaction that was conducted under identical conditions on the same scale. The combined reaction mixtures were diluted with diethyl ether (150 ml) and water (50 ml), and the mixture was stirred for 10 min at RT. The suspension was filtered over Celite, and the filter cake was washed with diethyl ether. The filtrates were combined, the layers were separated, and the organic layer was dried over magnesium sulfate and filtered. The filtrate was evaporated, and the residue was purified by column chromatography on silica gel (100 g silica gel; eluent: gradient of ethyl acetate in cyclohexane) to afford the title compound. Yield: 3.00 g (74% of theory).

LC/MS [Method 7]: Rₜ=0.80 min; MS (ESIpos): m/z=255 [M+H]⁺.

¹H-NMR (400 MHz, DMSO-d₆): δ [ppm]=8.74 (d, 1H), 8.21 (dd, 1H), 6.92 (d, 1H), 3.93 (s, 3H), 2.71 (s, 3H), 1.21 (s, 9H).

### Intermediate 48A

### (S)—N-[(1R)-1-(6-Methoxypyridin-3-yl)ethyl]-2-methylpropane-2-sulfinamide

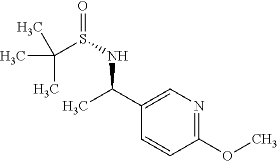 [SMILES: COc1ccc([C@@H](C)N[S@@](=O)C(C)(C)C)cn1]

Lithium tri-sec.-butylborohydride (L-Selectride®, 12.0 ml, 1.0 M solution in THF, 12.0 mmol) was added dropwise to a solution of (S)—N-[(1E)-1-(6-methoxypyridin-3-yl)ethylidene]-2-methylpropane-2-sulfinamide (Intermediate 47A, 3.00 g, 11.8 mmol) in THF (90 ml) at −78° C. The mixture was stirred at this temperature for 90 min before 100 ml of a saturated aqueous ammonium chloride solution was added. The mixture was diluted with water (50 ml) and extracted with ethyl acetate (3×50 ml). The combined organic layers were washed with brine, dried over magnesium sulfate and filtered. The filtrate was concentrate under reduced pressure, and the residue was purified by column chromatography on silica gel (100 g silica gel; eluent: gradient of methanol in dichloromethane) to afford the title compound. Yield: 2.36 g (76% of theory).

LC/MS [Method 3]: Rₜ=1.30 min; MS (ESIpos): m/z=257 [M+H]⁺.

¹H-NMR (400 MHz, DMSO-d₆): δ [ppm]=8.10 (d, 1H), 7.67 (dd, 1H), 6.79 (d, 1H), 5.37 (d, 1H), 4.43-4.36 (m, 1H), 3.83 (s, 3H), 1.45 (d, 3H), 1.09 (s, 9H).

### Intermediate 49A

### (1R)-1-(6-Methoxypyridin-3-yl)ethanamine

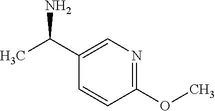

A solution of (S)—N-[(1R)-1-(6-methoxypyridin-3-yl)ethyl]-2-methylpropane-2-sulfinamide (Intermediate 48A, 2.36 g, 9.21 mmol) in methanol (23.0 ml) was cooled to 0° C. Hydrogen chloride (23.0 ml, 4.0 M solution in 1,4-dioxane, 92.0 mmol) was added dropwise at this temperature. After complete addition, the mixture was stirred at RT for 1 h. The mixture was poured into saturated aqueous sodium bicarbonate solution and extracted with ethyl acetate. The organic layer was discarded, and the aqueous layer was treated with 1.0 M aqueous sodium hydroxide solution until pH>10 was reached. The aqueous phase was extracted with dichloromethane, and the combined organic layers were dried over magnesium sulfate, filtered and evaporated to dryness to afford the title compound which was directly used in the next step without further purification. Yield: 860 mg (61% of theory).

¹H-NMR (400 MHz, DMSO-d₆): δ [ppm]=8.09 (d, 1H), 7.71 (dd, 1H), 6.75 (d, 1H), 3.97 (q, 1H), 3.57 (s, 3H), 1.97 (br. s, 2H), 1.23 (d, 3H).

### Intermediate 50A

### rac-1-(2-Naphthyl)propan-1-ol

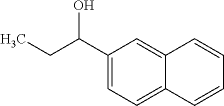

To a solution of 2-naphthaldehyde (10.00 g, 64.0 mmol) in THF (150 ml) was added ethyl-magnesium bromide (36.0 ml, 108.8 mmol, 3.0 M solution in diethyl ether) at −78° C. After stirring for 2 hours at room temperature, the reaction mixture was quenched with brine and extracted with ethyl acetate. The combined organic layers were dried over anhydrous sodium sulfate and filtered. The filtrate was concentrated under reduced pressure, and the residue was purified by flash-chromatography on silica gel (eluent: petroleum ether/ethyl acetate 3:1) to give 10.50 g (86% of theory) of the title compound.

LC/MS [Method 5]: Rₜ=1.08 min; MS (ESIpos): m/z=169 [M+H×H₂O]⁺.

¹H-NMR (400 MHz, DMSO-d₆): δ [ppm]=7.80-7.89 (m, 4H), 7.44-7.51 (m, 3H), 5.26 (d, 1H), 4.60-4.64 (m, 1H), 1.67-1.74 (m, 2H), 0.85 (t, 3H).

### Intermediate 51A

### 1-(2-Naphthyl)propan-1-one

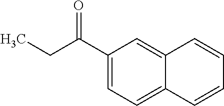

To a solution of 1-(2-naphthyl)propan-1-ol (Intermediate 50A, 10.50 g, 55.3 mmol) in dichloromethane (200 ml) was added Dess-Martin periodinane (46.86 g, 110.5 mmol). After stirring overnight at room temperature, the reaction mixture was concentrated under reduced pressure. The residue was purified by flash-chromatography on silica gel (eluent: petroleum ether/ethyl acetate 7:3) to give 10.30 g (98% of theory) of the title compound.

LC/MS [Method 20]: Rₜ=1.08 min; MS (ESIpos): m/z=185 [M+H]⁺.

¹H-NMR (300 MHz, DMSO-d₆): δ [ppm]=8.68 (s, 1H), 8.13 (d, 1H), 7.97-8.04 (m, 3H), 7.59-7.70 (m, 2H), 3.20 (q, 2H), 1.15 (t, 3H).

### Intermediate 52A

### 2-Bromo-1-(2-naphthyl)propan-1-one

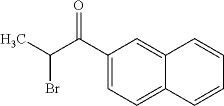

To a solution of 1-(2-naphthyl)propan-1-one (Intermediate 51A, 10.30 g, 54.6 mmol) in chloroform (200 ml) was added phenyltrimethylammonium tribromide (20.53 g, 54.6 mmol). After stirring overnight at room temperature, the reaction mixture was concentrated under reduced pressure. The residue was purified by flash-chromatography on silica gel (eluent: petroleum ether/ethyl acetate 4:1) to give 12.50 g (82% of theory, 94% purity) of the title compound.

LC/MS [Method 20]: Rₜ=1.15 min; MS (ESIpos): m/z=263/265 [M+H]⁺.

¹H-NMR (300 MHz, DMSO-d₆): δ [ppm]=8.79 (s, 1H), 7.97-8.20 (m, 4H), 7.62-7.76 (m, 2H), 6.01 (q, 1H), 1.85 (d, 3H).

### Intermediate 53A

### Ethyl (2Z)-3-[(trifluoromethyl)sulfonyl]but-2-enoate

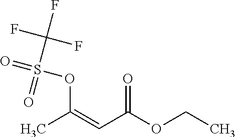 [SMILES: CCOC(=O)/C=C(/C)OS(=O)(=O)C(F)(F)F]

To a solution of ethyl 3-oxobutanoate (20.00 g, 153.7 mmol) in toluene (800 ml) was added a solution of lithium hydroxide (27.60 g, 1152.6 mmol) in water (232 ml) at 10° C. After stirring for 5 minutes at 10° C., trifluoromethanesulfonic anhydride (52.0 ml, 307.4 mmol) was added dropwise. The resulting mixture was stirred for 1.5 hours below 25° C. and then poured into water (800 ml) and extracted with ethyl acetate. The combined organic layers were washed with water and brine, dried over anhydrous sodium sulfate and filtered. The filtrate was concentrated under reduced pressure to give 25.00 g (62% of theory) of the title compound which was used in the next step without further purification.

¹H-NMR (400 MHz, DMSO-d₆): δ [ppm]=6.27 (s, 1H), 4.15 (q, 2H), 2.19 (s, 3H), 1.22 (t, 3H).

### Intermediate 54A

### Diethyl 4-methyl-1H-pyrazole-3,5-dicarboxylate

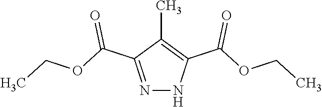 [SMILES: CCOC(=O)c1n[nH]c(C(=O)OCC)c1C]

A mixture of ethyl (2Z)-3-[(trifluoromethyl)sulfonyl]but-2-enoate (Intermediate 53A, 25.00 g, 95.3 mmol), tetrakis(triphenylphosphine)palladium(0) (5.51 g, 4.8 mmol), 4-methylmorpholine (20.97 ml, 190.7 mmol) and ethyl 2-diazoacetate (15.0 ml, 143.0 mmol) in N,N-dimethylformamide (250 ml) was stirred at room temperature for 1.5 hours under a nitrogen atmosphere. When the ethyl (2Z)-3-[(trifluoromethyl)sulfonyl]but-2-enoate was consumed (as monitored by TLC and LC/MS), the reaction mixture was heated to 60° C. and stirred at this temperature overnight. After cooling to room temperature, the reaction mixture was poured into water (300 ml) and extracted with ethyl acetate. The combined organic layers were washed with water and brine, dried over anhydrous sodium sulfate and filtered. The filtrate was concentrated under reduced pressure, and the residue was purified by flash-chromatography on silica gel (eluent: petroleum ether/ethyl acetate 5:1) to give 12.30 g (56% of theory) of the title compound.

LC/MS [Method 21]: Rₜ=1.16 min; MS (ESIpos): m/z=453 [2M+H]⁺.

¹H-NMR (300 MHz, DMSO-d₆): δ [ppm]=14.30 (s, 1H), 4.24-4.37 (m, 4H), 2.45 (s, 3H), 1.24-1.35 (m, 6H).

### Intermediate 55A

### Diethyl 4-methyl-1-[1-(2-naphthyl)-1-oxopropan-2-yl]-1H-pyrazole-3,5-dicarboxylate

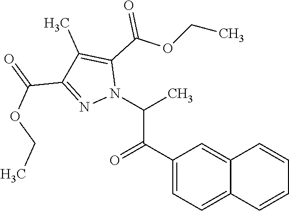 [SMILES: CCOC(=O)c1nn(C(C)C(=O)c2ccc3ccccc3c2)c(C(=O)OCC)c1C]

To a solution of 2-bromo-1-(2-naphthyl)propan-1-one (Intermediate 52A, 6.8 g, 20.9 mmol, 81% purity) in acetone (100 ml) were added diethyl 4-methyl-1H-pyrazole-3,5-dicarboxylate (Intermediate 54A, 4.35 g, 19.03 mmol) and potassium carbonate (6.58 g, 47.6 mmol). After stirring at room temperature overnight, the solids were filtered off, and the filtrate was concentrated under reduced pressure. The residue was purified by flash-chromatography on silica gel (330 g silica gel; eluent: petroleum ether/ethyl acetate 3:1) to give 8.65 g (97% of theory, 96% purity) of the title compound.

LC/MS [Method 5]: Rₜ=1.33 min; MS (ESIpos): m/z=409 [M+H]⁺.

¹H-NMR (300 MHz, DMSO-d₆): δ [ppm]=8.76 (s, 1H), 7.97-8.16 (m, 4H), 7.64-7.74 (m, 2H), 6.98-7.06 (m, 1H), 4.32 (q, 2H), 4.19 (q, 2H), 2.49 (s, 3H), 1.81 (d, 3H), 1.32 (t, 3H), 1.14 (t, 3H).

### Intermediate 56A

### Ethyl 3,7-dimethyl-6-(2-naphthyl)-4-oxo-4,5-dihydropyrazolo[1,5-a]pyrazine-2-carboxylate

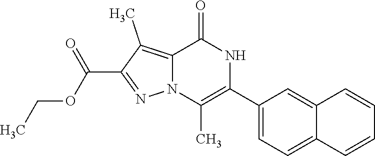 [SMILES: CCOC(=O)c1nn2c(C)c(-c3ccc4ccccc4c3)[nH]c(=O)c2c1C]

Ammonium acetate (31.5 g, 408.7 mol) was added to a solution of diethyl 4-methyl-1-[1-(2-naphthyl)-1-oxopropan-2-yl]-1H-pyrazole-3,5-dicarboxylate (Intermediate 55A, 8.65 g, 20.4 mmol) in acetic acid (120 ml) at room temperature. The reaction mixture was gradually warmed to 110° C. and stirred at this temperature for 18 h. After cooling to RT, the solution was diluted with water (300 ml). The precipitate was collected by filtration, washed with water (500 ml) and dried to afford 7.20 g (87% of theory, 89% purity) of the title compound.

LC/MS [Method 10]: Rₜ=1.14 min; MS (ESIpos): m/z=362 [M+H]⁺.

¹H-NMR (300 MHz, DMSO-d₆): δ [ppm]=11.52 (s, 1H), 8.01-8.10 (m, 4H), 7.58-7.65 (m, 3H), 4.37 (q, 2H), 2.69 (s, 3H), 2.36 (s, 3H), 1.35 (t, 3H).

### Intermediate 57A

### 3,7-Dimethyl-6-(2-naphthyl)-4-oxo-4,5-dihydropyrazolo[1,5-a]pyrazine-2-carboxylic acid

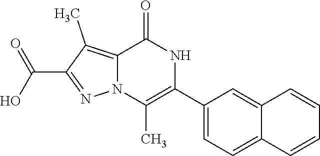 [SMILES: Cc1c(C(=O)O)nn2c(C)c(-c3ccc4ccccc4c3)[nH]c(=O)c12]

To a solution of ethyl 3,7-dimethyl-6-(2-naphthyl)-4-oxo-4,5-dihydropyrazolo[1,5-a]pyrazine-2-carboxylate (Intermediate 56A, 7.2 g, 17.8 mmol) in ethanol (100 ml) was added a solution of sodium hydroxide (7.12 g, 177.9 mmol) in water (50 ml). The reaction mixture was stirred at room temperature for 2 h. The solution was then diluted with water (100 ml) and extracted with ethyl acetate (3×100 ml). The combined organic layers were discarded, and the aqueous layer was adjusted to pH 3 with 3.0 M hydrochloric acid. The precipitate was collected by filtration and dried to afford 5.48 g (90% of theory, 98% purity) of the title compound.

LC/MS [Method 22]: Rₜ=1.18 min; MS (ESIpos): m/z=334 [M+H]⁺.

¹H-NMR (300 MHz, DMSO-d₆): δ [ppm]=13.10 (br. s, 1H), 11.47 (s, 1H), 8.01-8.10 (m, 4H), 7.60-7.63 (m, 3H), 2.68 (s, 3H), 2.35 (s, 3H).

### Intermediate 58A

### 1-Bromo-4-[2-(methoxymethoxy)ethyl]benzene

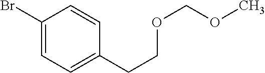

To a mixture of 2-(4-bromophenyl)ethanol (20.00 g, 99.5 mmol) and N,N-diisopropylethylamine (33.0 ml, 198.9 mmol) in dry dichloromethane (200 ml) was added bromomethyl methyl ether (12.2 ml, 149.2 mmol) dropwise at 0° C. After stirring at room temperature for 2.5 h under a nitrogen atmosphere, the reaction mixture was washed with water, dried over sodium sulfate and filtered. The filtrate was concentrated under vacuum, and the residue was purified by flash-chromatography on silica gel (eluent: petroleum ether/ethyl acetate 3:1) to give 15.40 g (38% of theory, 61% purity) of the title compound.

¹H-NMR (300 MHz, DMSO-d₆): δ [ppm]=7.42-7.52 (m, 2H), 7.19-7.26 (m, 2H), 4.54 (s, 2H), 3.66 (t, 2H), 3.17 (s, 3H), 2.81 (t, 2H).

### Intermediate 59A

### 7-Bromo-3,4-dihydro-1H-isochromene

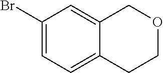 [SMILES: Brc1ccc2c(c1)COCC2]

Trimethylsilyl trifluoromethanesulfonate (6.9 ml, 38.3 mmol) was added dropwise to a solution of 1-bromo-4-[2-(methoxymethoxy)ethyl]benzene (Intermediate 58A, 15.40 g, 38.3 mmol, 61% purity) in dry acetonitrile (160 ml) at 0° C. under a nitrogen atmosphere. After stirring for 3 h at 0° C., the reaction mixture was quenched by addition of aqueous 1.0 M sodium bicarbonate solution. The phases were separated, and the organic phase was washed with brine, dried over sodium sulfate and filtered. The filtrate was evaporated under reduced pressure, and the residue was purified by flash-chromatography on silica gel (eluent: petroleum ether/ethyl acetate 5:1) to give 7.10 g (60% of theory, 70% purity) of the title compound.

¹H-NMR (300 MHz, DMSO-d₆): δ [ppm]=7.34 (d, 1H), 7.20-7.27 (m, 2H), 4.66 (s, 2H), 3.87 (t, 2H), 2.75 (t, 2H).

### Intermediate 60A

### Methyl 3,4-dihydro-1H-isochromene-7-carboxylate

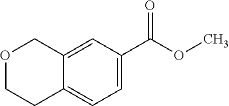

A mixture of 7-bromo-3,4-dihydro-1H-isochromene (Intermediate 59A, 7.10 g, 23.3 mmol, 70% purity), triethylamine (7.1 ml, 70.0 mmol) and 1,1′-bis(diphenylphosphino)ferrocene-palladium(II) dichloride dichloromethane complex (1.91 g, 2.3 mmol) in methanol (80 ml) and N,N-dimethyl-formamide (40 ml) was stirred for 16 hat 100° C. under a carbon monoxide (5 atm) atmosphere in an autoclave. After cooling to room temperature, the reaction mixture was concentrated under reduced pressure. The residue was purified by flash-chromatography on silica gel (eluent: petroleum ether/ethyl acetate 3:1) to give 3.50 g (63% of theory, 81% purity) of the title compound.

LC/MS [Method 12]: Rₜ=0.98 min; MS (ESIpos): m/z=193 [M+H]⁺.

¹H-NMR (300 MHz, DMSO-d₆): δ [ppm]=7.76 (d, 1H), 7.66 (s, 1H), 7.42 (d, 1H), 4.74 (s, 2H), 3.90 (t, 2H), 3.84 (s, 3H), 2.87 (t, 2H).

### Intermediate 61A

### 3,4-Dihydro-1H-isochromene-7-carboxylic acid

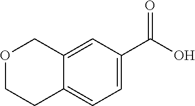 [SMILES: O=C(O)c1ccc2c(c1)COCC2]

To a solution of methyl 3,4-dihydro-1H-isochromene-7-carboxylate (Intermediate 60A, 3.50 g, 14.7 mmol, 81% purity) in methanol (50 ml) and water (30 ml) was added sodium hydroxide (2.95 g, 73.7 mmol). After stirring at room temperature for 2 h, the reaction mixture was diluted with water and extracted with ethyl acetate. The organic layer was discarded, and the aqueous layer was adjusted to pH 3 with 1.0 M hydrochloric acid. The precipitate was collected by filtration and dried in vacuo to give 2.20 g (75% of theory, 90% purity) of the title compound.

¹H-NMR (300 MHz, DMSO-d₆): δ [ppm]=12.84 (s, 1H), 7.71-7.74 (m, 1H), 7.63 (s, 1H), 7.25 (d, 1H), 4.73 (s, 2H), 3.89 (t, 2H), 2.84 (t, 2H).

### Intermediate 62A

### 3,4-Dihydro-1H-isochromene-7-carbonyl chloride

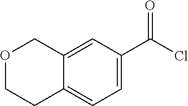 [SMILES: O=C(Cl)c1ccc2c(c1)COCC2]

3,4-Dihydro-1H-isochromene-7-carboxylic acid (Intermediate 61A, 2.20 g, 11.1 mmol, 90% purity) was dissolved in thionyl chloride (25.0 ml) and heated to reflux for 2 h. The thionyl chloride was then removed under reduced pressure to give 2.30 g (95% of theory, 90% purity) of the title compound which was directly used for the next step without further purification.

¹H-NMR (300 MHz, DMSO-d₆): δ [ppm]=7.71-7.78 (m, 1H), 7.63 (s, 1H), 7.24-7.30 (m, 1H), 4.73-4.78 (m, 2H), 3.87-3.93 (m, 2H), 2.82-2.93 (m, 2H).

### Intermediate 63A

### 2-Bromo-1-(3,4-dihydro-1H-isochromen-7-yl)ethanone

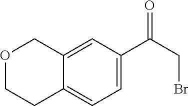 [SMILES: O=C(CBr)c1ccc2c(c1)COCC2]

A solution of (trimethylsilyl)diazomethane in diethyl ether (10.5 ml, 2.0 M, 21.0 mmol) was added to a stirred solution of 3,4-dihydro-1H-isochromene-7-carbonyl chloride (Intermediate 62A, 2.30 g, 10.5 mmol, 90% purity) in tetrahydrofuran (20 ml) and acetonitrile (20 ml) at room temperature. After stirring for 1 h, 40% hydrobromic acid aqueous solution (6.0 ml, 44.0 mmol) was added at 0° C. The resulting mixture was stirred for an additional 30 minutes at room temperature. Then, saturated sodium bicarbonate solution was added carefully until pH>7 was reached. The layers were separated, and the aqueous phase was extracted with ethyl acetate. The combined organic layers were washed with water and brine, dried over anhydrous sodium sulfate and filtered. The filtrate was concentrated under reduced pressure, and the residue was purified by flash-chromatography on silica gel (eluent: petroleum ether/ethyl acetate 4:1) to give 2.00 g (67% of theory, 90% purity) of the title compound.

¹H-NMR (300 MHz, DMSO-d₆): δ [ppm]=7.76-7.82 (m, 1H), 7.74 (s, 1H), 7.32 (d, 1H), 4.88 (s, 2H), 4.75 (s, 2H), 3.89-3.92 (m, 2H), 2.85-2.91 (m, 2H).

### Intermediate 64A

### Diethyl 1-[2-(3,4-dihydro-1H-isochromen-7-yl)-2-oxoethyl]-1H-pyrazole-3,5-dicarboxylate

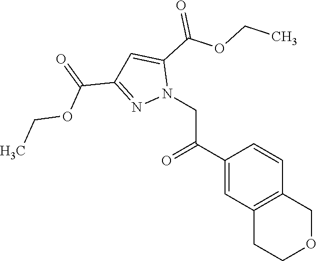 [SMILES: CCOC(=O)c1cc(C(=O)OCC)n(CC(=O)c2ccc3c(c2)CCOC3)n1]

To a solution of 2-bromo-1-(3,4-dihydro-1H-isochromen-7-yl)ethanone (Intermediate 63A, 2.30 g, 8.1 mmol, 90% purity) in acetone (35 ml) were added diethyl 1H-pyrazole-3,5-dicarboxylate (1.56 g, 7.4 mmol) and potassium carbonate (2.75 g, 19.9 mmol). After stirring overnight at room temperature. the solids were filtered off, and the filtrate was concentrated under reduced pressure. The residue was purified by flash-chromatography on silica gel (eluent: petroleum ether/ethyl acetate 4:1) to give 2.40 g (83% of theory) of the title compound.

LC/MS [Method 23]: Rₜ=0.91 min; MS (ESIpos): m/z=387 [M+H]⁺.

¹H-NMR (400 MHz, DMSO-d₆): δ [ppm]=7.84-7.86 (m, 1H), 7.76 (s, 1H), 7.35-7.38 (m, 2H), 6.20 (s, 2H), 4.77 (s, 2H), 4.31 (q, 2H), 4.20 (q, 2H), 3.92 (t, 2H), 2.89 (t, 2H), 1.31 (t, 3H), 1.17 (t, 3H).

### Intermediate 65A

### Ethyl 6-(3,4-dihydro-1H-isochromen-7-yl)-4-oxo-4,5-dihydropyrazolo[1,5-a]pyrazine-2-carboxylate

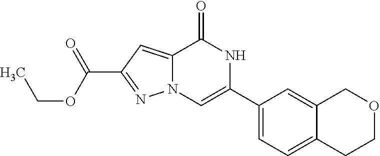 [SMILES: CCOC(=O)c1cc2c(=O)[nH]c(-c3ccc4c(c3)COCC4)cn2n1]

To a solution of diethyl 1-[2-(3,4-dihydro-1H-isochromen-7-yl)-2-oxoethyl]-1H-pyrazole-3,5-dicarboxylate (Intermediate 64A, 2.40 g, 6.2 mmol) in acetic acid (50 ml) was added ammonium acetate (9.57 g, 124.2 mmol), and the mixture was stirred at 110° C. for 72 h. After cooling to room temperature, the reaction mixture was poured into ice-water. The solids were collected by filtration, washed with water and dried in vacuo to give 2.00 g (60% of theory) of the title compound, which contained 31% ester hydrolysis by-product (see Intermediate 66A) and was used as such for the next step without further purification. Yield: 91% of theory (60% of the ester and 31% of the acid).

¹H-NMR (300 MHz, DMSO-d₆): δ [ppm]=8.14 (s, 1H), 7.40-7.56 (m, 3H), 7.23-7.27 (m, 2H), 4.73 (s, 2H), 4.35 (q, 2H), 3.91 (t, 2H), 2.83 (t, 2H), 1.33 (t, 3H).

### Intermediate 66A

### 6-(3,4-Dihydro-1H-isochromen-7-yl)-4-oxo-4,5-dihydropyrazolo[1,5-a]pyrazine-2-carboxylic acid

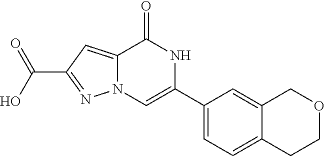 [SMILES: O=C(O)c1cc2c(=O)[nH]c(-c3ccc4c(c3)COCC4)cn2n1]

To 2.00 g of the mixture of ethyl 6-(3,4-dihydro-1H-isochromen-7-yl)-4-oxo-4,5-dihydropyrazolo-[1,5-a]pyrazine-2-carboxylate (3.7 mmol) and 6-(3,4-dihydro-1H-isochromen-7-yl)-4-oxo-4,5-dihydropyrazolo[1,5-a]pyrazine-2-carboxylic acid (2.0 mmol) from Intermediate 65A in ethanol (36 ml) and water (4.0 ml) was added sodium hydroxide (2.36 g, 58.9 mmol). After stirring at room temperature for 4 h, the reaction mixture was diluted with water and extracted with ethyl acetate. The aqueous layer was adjusted to pH 3 with 1.0 M hydrochloric acid. The precipitate was collected by filtration and dried in vacuo to give 1.26 g (70% of theory, 92% purity) of the title compound.

LC/MS [Method 2]: Rₜ=0.89 min; MS (ESIpos): m/z=312 [M+H]⁺.

¹H-NMR (400 MHz, DMSO-d₆): δ [ppm]=13.26 (br. s, 1H), 11.72 (s, 1H), 8.11 (s, 1H), 7.54-7.56 (m, 1H), 7.46 (s, 1H), 7.36 (s, 1H), 7.26 (d, 1H), 4.73 (s, 2H), 3.91 (t, 2H), 2.83 (t, 2H).

### Intermediate 67A

### Methyl 2-[(2-bromophenyl)(hydroxy)methyl]acrylate

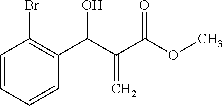

A mixture of 2-bromobenzaldehyde (5.00 g, 27.0 mmol), methyl acrylate (6.98 g, 81.1 mmol) and 1,4-diazabicyclo[2.2.2]octane (3.03 g, 27.0 mmol) was stirred at room temperature for two days. The reaction solution was then diluted with water (100 ml) and extracted with dichloromethane (3×100 ml). The combined organic layers were dried over anhydrous magnesium sulfate and filtered. The filtrate was concentrated under reduced pressure, and the residue was purified by flash-chromatography on silica gel (eluent: hexane/ethyl acetate 10:1) to give 7.20 g (95% of theory, 96% purity) of the title compound.

LC/MS [Method 10]: Rₜ=0.96 min; MS (ESIpos): m/z=294/296 [M+Na]⁺.

¹H-NMR (300 MHz, DMSO-d₆): δ [ppm]=7.56-7.59 (m, 1H), 7.37-7.40 (m, 2H), 7.18-7.24 (m, 1H), 6.21-6.23 (m, 1H), 5.88 (d, 1H), 5.75 (d, 1H), 5.60-5.62 (m, 1H), 3.64 (s, 3H).

### Intermediate 68A

### Methyl 2-[acetoxy(2-bromophenyl)methyl]acrylate

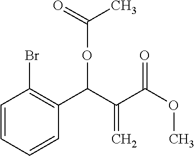 [SMILES: C=C(C(=O)OC)C(OC(C)=O)c1ccccc1Br]

To a stirred solution of methyl 2-[(2-bromophenyl)(hydroxy)methyl]acrylate (Intermediate 67A, 6.20 g, 22.9 mmol) in dichloromethane (50 ml) were added acetic anhydride (3.24 ml, 34.3 mmol) and 4-N,N-dimethylaminopyridine (560 mg, 4.6 mmol) at room temperature. After stirring overnight at room temperature, the reaction mixture was diluted with water (100 ml) and extracted with dichloromethane (3×100 ml). The combined organic layers were dried over anhydrous magnesium sulfate and filtered. The filtrate was concentrated under reduced pressure, and the residue was purified by flash-chromatography on silica gel (eluent: hexane/ethyl acetate 10:1) to give 5.63 g (74% of theory) of the title compound.

¹H-NMR (300 MHz, DMSO-d₆): δ [ppm]=7.68-7.71 (m, 1H), 7.31-7.48 (m, 3H), 6.81 (s, 1H), 6.44 (s, 1H), 5.65 (s, 1H), 3.71 (s, 3H), 2.12 (s, 3H).

### Intermediate 69A

### Methyl (2E)-2-(azidomethyl)-3-(2-bromophenyl)acrylate

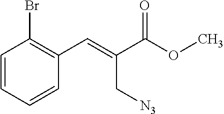 [SMILES: COC(=O)/C(=C/c1ccccc1Br)CN=[N+]=[N-]]

To a solution of methyl 2-[acetoxy(2-bromophenyl)methyl]acrylate (Intermediate 68A, 4.26 g, 13.6 mmol) in tetrahydrofuran (150 ml) and water (150 ml) was added sodium azide (1.77 g, 27.2 mmol). The resulting mixture was stirred at room temperature overnight and then extracted with dichloromethane (3×50 ml). The combined organic layers were dried over anhydrous magnesium sulfate and filtered. The filtrate was concentrated in vacuum, and the residue was purified by flash-chromatography on silica gel (eluent: petroleum ether/ethyl acetate 4:1) to give 2.20 g (53% of theory, 97% purity) of the title compound.

LC/MS [Method 9]: Rₜ=1.29 min; MS (ESIpos): m/z=268/270 [M+H−N₂]⁺.

¹H-NMR (300 MHz, DMSO-d₆): δ [ppm]=7.83 (s, 1H), 7.72-7.75 (m, 1H), 7.46-7.51 (m, 1H), 7.34-7.39 (m, 2H), 4.08 (s, 2H), 3.82 (s, 3H).

### Intermediate 70A

### Methyl 5-bromoquinoline-3-carboxylate

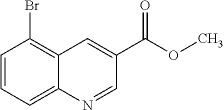

Methyl (2E)-2-(azidomethyl)-3-(2-bromophenyl)acrylate (Intermediate 69A, 5.50 g, 18.6 mmol) and N-bromosuccinimide (6.60 g, 37.2 mmol) were dissolved in dry dichloromethane (550 ml) in a 1000 ml round-bottom flask. The resulting solution was irradiated with a household fluorescent lamp (60 W) at ambient temperature for 2 hours. For work-up, this reaction mixture was combined with a second reaction that was conducted under identical conditions on the same scale. From the combined mixtures, the solvent was removed under reduced pressure, and the residue was purified by flash-chromatography on silica gel (eluent: petroleum ether/ethyl acetate 15:1→8:1) to give 7.62 g (76% of theory) of the title compound.

LC/MS [Method 20]: Rₜ=1.00 min; MS (ESIpos): m/z=266/268 [M+H]⁺.

¹H-NMR (300 MHz, DMSO-d₆): δ [ppm]=9.37 (s, 1H), 9.00 (s, 1H), 8.10-8.19 (m, 2H), 7.85-7.90 (m, 1H), 4.00 (s, 3H).

### Intermediate 71A

### Methyl 5-methylquinoline-3-carboxylate

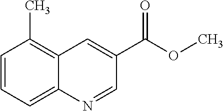

A mixture of methyl 5-bromoquinoline-3-carboxylate (Intermediate 70A, 9.10 g, 34.2 mmol), tetrakis(triphenylphosphine)palladium(0) (3.95 g, 3.4 mmol), potassium carbonate (11.8 g, 85.5 mmol) and methylboronic acid (6.14 g, 103 mmol) in 1,4-dioxane (250 ml) was stirred overnight at 110° C. under a nitrogen atmosphere. After cooling to room temperature, the reaction mixture was concentrated under reduced pressure. The residue was purified by flash-chromatography on silica gel (eluent: petroleum ether/ethyl acetate 5:1) to give 6.04 g (87% of theory) of the title compound.

¹H-NMR (300 MHz, DMSO-d₆): δ [ppm]=9.31 (s, 1H), 8.93 (s, 1H), 7.94-7.96 (m, 1H), 7.79-7.83 (m, 1H), 7.55-7.57 (m, 1H), 3.97 (s, 3H), 2.73 (s, 3H).

### Intermediate 72A

### 5-Methylquinoline-3-carboxylic acid hydrochloride

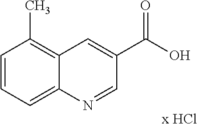

To a solution of methyl 5-methylquinoline-3-carboxylate (Intermediate 71A, 6.04 g, 30.0 mmol) in methanol (100 ml) was added 3.0 M aqueous sodium hydroxide solution (5.0 ml), and the resulting mixture was stirred for 2 hours at room temperature. 3.0 M hydrochloric acid was then added until pH 1 was reached, and the mixture was concentrated under reduced pressure. The residue was dissolved in methanol (100 ml), and the remaining solids were filtered off. The filtrate was evaporated under reduced pressure to give 5.60 g (83% of theory) of the title compound.

¹H-NMR (300 MHz, DMSO-d₆): δ [ppm]=12.11 (br. s, 1H), 9.36 (s, 1H), 9.19 (s, 1H), 8.15 (d, 1H), 7.92-7.97 (m, 1H), 7.67-7.70 (m, 1H), 2.77 (s, 3H).

### Intermediate 73A

### 5-Methylquinoline-3-carbonyl chloride hydrochloride

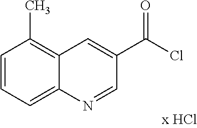

A mixture of 5-methylquinoline-3-carboxylic acid hydrochloride (Intermediate 72A, 5.62 g, 25.1 mmol) and thionyl chloride (100 ml) was refluxed for 2 h. After cooling to RT, the volatiles were removed under reduced pressure to afford the crude title compound which was directly used for the next step without further purification. Yield: 6.00 g (98% of theory).

¹H-NMR (300 MHz, DMSO-d₆): δ [ppm]=9.39 (s, 1H), 9.15 (s, 1H), 8.07 (d, 1H), 7.89-7.94 (m, 1H), 7.66 (d, 1H), 2.77 (s, 3H).

### Intermediate 74A

### 2-Bromo-1-(5-methylquinolin-3-yl)ethanone and 2-chloro-1-(5-methylquinolin-3-yl)ethanone

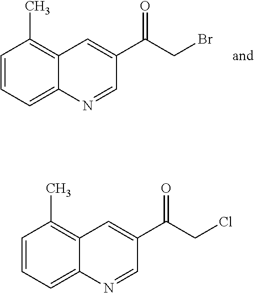

To a stirred solution of 5-methylquinoline-3-carbonyl chloride hydrochloride (Intermediate 73A, 17.80 g, 58.8 mmol, purity 80%) in tetrahydrofuran (240 ml) and acetonitrile (240 ml) was added (trimethylsilyl)diazomethane solution (58.8 ml, 118 mmol, 2.0 M in diethyl ether) dropwise at room temperature. After stirring for 1 h, hydrobromic acid (20.0 ml, 40% solution in water) was added at 0° C., and the mixture was stirred for an additional 30 minutes at 0° C. The reaction solution was then adjusted to pH 8 with saturated aqueous sodium bicarbonate solution and extracted with ethyl acetate. The combined organic layers were washed with water and brine and dried over anhydrous sodium sulfate. The mixture was filtered, and the filtrate was concentrated to afford a mixture (8.64 g) of the crude title compounds which was directly used for the next step without further purification. In the next step, it was assumed that the obtained material consisted of pure 2-bromo-1-(5-methylquinolin-3-yl)ethanone.

LC/MS [Method 24]: Rₜ=2.44 min; MS (ESIpos): m/z=264/266 [M+H]⁺, and Rₜ=2.21 min; MS (ESIpos): m/z=220 [M+H]⁺.

### Intermediate 75A

### Diethyl 4-isopropyl-1-[2-(5-methylquinolin-3-yl)-2-oxoethyl]-1H-pyrazole-3,5-dicarboxylate

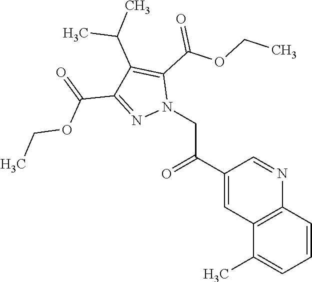 [SMILES: CCOC(=O)c1nn(CC(=O)c2cnc3cccc(C)c3c2)c(C(=O)OCC)c1C(C)C]

To a solution of diethyl 4-isopropyl-1H-pyrazole-3,5-dicarboxylate (Intermediate 16A, 5.16 g, 20.3 mmol) in acetone (120 ml) were added 2-bromo-1-(5-methylquinolin-3-yl)ethanone (Intermediate 74A, 5.36 g, 20.3 mmol) and potassium carbonate (8.41 g, 60.9 mmol). The resulting mixture was stirred at room temperature for 6 h. After filtering off the solids, the filtrate was concentrated under reduced pressure. The residue was purified by flash-chromatography on silica gel (330 g silica gel; eluent: petroleum ether/ethyl acetate 6:1) to give 5.98 g (63% of theory, 91% purity) of the title compound.

LC/MS [Method 25]: Rₜ=1.25 min; MS (ESIpos): m/z=438 [M+H]⁺.

¹H-NMR (300 MHz, CDCl₃): δ [ppm]=9.41 (d, 1H), 8.91 (d, 1H), 8.06 (d, 1H), 7.75-7.80 (m, 1H), 7.50 (d, 1H), 6.16 (s, 2H), 4.40 (q, 2H), 4.31 (q, 2H), 3.85-3.98 (m, 1H), 2.79 (s, 3H), 1.51-1.37 (m, 9H), 1.35 (t, 3H).

### Intermediate 76A

### Ethyl 3-isopropyl-6-(5-methylquinolin-3-yl)-4-oxo-4,5-dihydropyrazolo[1,5-a]pyrazine-2-carboxylate

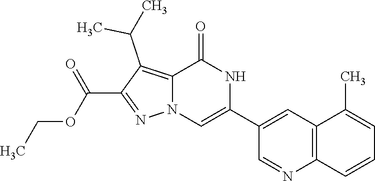 [SMILES: CCOC(=O)c1nn2cc(-c3cnc4cccc(C)c4c3)[nH]c(=O)c2c1C(C)C]

To a solution of diethyl 4-isopropyl-1-[2-(5-methylquinolin-3-yl)-2-oxoethyl]-1H-pyrazole-3,5-dicarboxylate (Intermediate 75A, 6.38 g, 13.3 mmol, 91% purity) in acetic acid (90 ml) was added ammonium acetate (20.5 g, 265 mmol). The resulting mixture was stirred at 120° C. overnight. After cooling to room temperature, the reaction mixture was poured into water (400 ml). The solid was collected by filtration, washed with water and dried in air to give 5.51 g (99% of theory, 93% purity) of the title compound.

LC/MS [Method 25]: Rₜ=1.06 min; MS (ESIpos): m/z=391 [M+H]⁺.

¹H-NMR (300 MHz, DMSO-d₆): δ [ppm]=9.27 (d, 1H), 8.85 (d, 1H), 8.54 (s, 1H), 7.90 (d, 1H), 7.68-7.74 (m, 1H), 7.52 (d, 1H), 4.36 (q, 2H), 4.13-4.18 (m, 1H), 2.77 (s, 3H), 1.32-1.41 (m, 9H).

### Intermediate 77A

### 3-Isopropyl-6-(5-methylquinolin-3-yl)-4-oxo-4,5-dihydropyrazolo[1,5-a]pyrazine-2-carboxylic acid

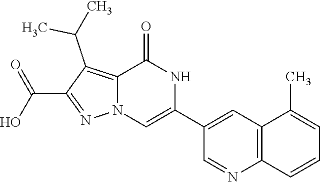 [SMILES: Cc1cccc2ncc(-c3cn4nc(C(=O)O)c(C(C)C)c4c(=O)[nH]3)cc12]

To a solution of ethyl 3-isopropyl-6-(5-methylquinolin-3-yl)-4-oxo-4,5-dihydropyrazolo[1,5-a]-pyrazine-2-carboxylate (Intermediate 76A, 5.51 g, 13.1 mmol, 93% purity) in ethanol (120 ml) was added a solution of sodium hydroxide (5.25 g, 131.2 mmol) in water (40 ml). The resulting mixture was stirred at room temperature for 3 h. The reaction mixture was then concentrated under reduced pressure to remove the ethanol. The remaining mixture was adjusted to pH 1 with 2.0 M hydrochloric acid. The precipitate was collected by filtration, washed with water and dried in air to afford 4.92 g (98% of theory, 97% pruity) of the title compound.

LC/MS [Method 9]: Rₜ=1.07 min; MS (ESIpos): m/z=363 [M+H]⁺.

¹H-NMR (300 MHz, DMSO-d₆): δ [ppm]=13.20 (br. s, 1H), 11.89 (s, 1H), 9.27 (d, 1H), 8.84 (d, 1H), 8.47 (s, 1H), 7.90 (d, 1H), 7.68-7.73 (m, 1H), 7.52 (d, 1H), 4.14-4.19 (m, 1H), 2.77 (s, 3H), 1.39 (d, 6H).

### Intermediate 78A

### Diethyl 4-iodo-1H-pyrazole-3,5-dicarboxylate

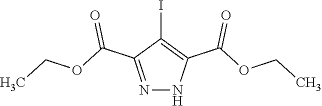 [SMILES: CCOC(=O)c1n[nH]c(C(=O)OCC)c1I]

To a solution of diethyl 1H-pyrazole-3,5-dicarboxylate (10.0 g, 47.1 mmol) in acetonitrile (150 ml) were added iodine (7.20 g, 28.3 mmol) and ammonium cerium(IV) nitrate (12.90 g, 23.6 mmol). The resulting mixture was stirred overnight at 85° C. After cooling to room temperature, the reaction mixture was quenched with saturated aqueous sodium thiosulfate solution (150 ml) and extracted with ethyl acetate. The combined organic layers were washed with water and brine, dried over anhydrous sodium sulfate and filtered. The filtrate was concentrated under reduced pressure, and the residue was purified by flash-chromatography on silica gel (eluent: petroleum ether/ethyl acetate 7:3) to give 15.0 g (94% of theory) of the title compound.

LC/MS [Method 26]: Rₜ=1.44 min; MS (ESIpos): m/z=339 [M+H]⁺.

¹H-NMR (300 MHz, DMSO-d₆): δ [ppm]=14.85 (s, 1H), 4.29-4.37 (m, 4H), 1.32-1.35 (m, 6H).

### Intermediate 79A

### Diethyl 4-iodo-1-[2-(2-naphthyl)-2-oxoethyl]-1H-pyrazole-3,5-dicarboxylate

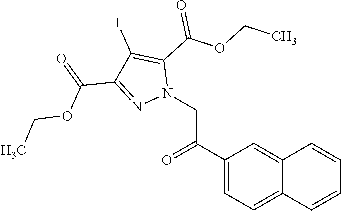 [SMILES: CCOC(=O)c1nn(CC(=O)c2ccc3ccccc3c2)c(C(=O)OCC)c1I]

To a solution of 2-bromo-1-(2-naphthyl)ethanone (42.0 g, 137.5 mmol, 82% purity) in acetone (800 ml) were added diethyl 4-iodo-1H-pyrazole-3,5-dicarboxylate (Intermediate 76A, 46.5 g, 91% purity, 125.0 mmol) and potassium carbonate (25.9 g, 187.5 mmol). After stirring at room temperature for 2.5 h, the solids were filtered off, and the filtrate was concentrated under reduced pressure to provide the crude product. This material was suspended in petroleum ether/ethyl acetate (20:1, 200 ml) and stirred for 30 minutes. Then, the resulting solid was collected by filtration, washed with petroleum ether/ethyl acetate (20:1, 2×50 ml) and dried to give 63.0 g (97% of theory, 98% purity) of the title compound.

LC/MS [Method 23]: Rₜ=1.12 min; MS (ESIpos): m/z=507 [M+H]⁺.

¹H-NMR (300 MHz, DMSO-d₆): δ [ppm]=8.81 (s, 1H), 7.98-8.25 (m, 4H), 7.65-7.75 (m, 2H), 6.41 (s, 2H), 4.34 (q, 2H), 4.20 (q, 2H), 1.32 (t, 3H), 1.14 (t, 3H).

### Intermediate 80A

### Ethyl 3-iodo-6-(2-naphthyl)-4-oxo-4,5-dihydropyrazolo[1,5-a]pyrazine-2-carboxylate

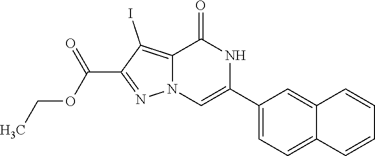 [SMILES: CCOC(=O)c1nn2cc(-c3ccc4ccccc4c3)[nH]c(=O)c2c1I]

To a solution of diethyl 4-iodo-1-[2-(2-naphthyl)-2-oxoethyl]-1H-pyrazole-3,5-dicarboxylate (Intermediate 79A, 31.5 g, 61.0 mmol) in acetic acid (500 ml) was added ammonium acetate (64.5 g, 836.2 mmol). The mixture was stirred for 6 h at 110° C. Then, more ammonium acetate (64.5 g, 836.2 mmol) was added to the reaction mixture, and stirring was continued at 120° C. overnight. A second batch of this reaction was performed under identical conditions and on the same scale.

After cooling to RT, both reaction mixtures were combined and poured into ice-water (2 Liter). The solid was collected by filtration, washed with water (2×500 ml) and dried to afford 55.0 g (93% of theory, 98% purity) of the title compound.

LC/MS [Method 12]: Rₜ=1.17 min; MS (ESIpos): m/z=482 [M+Na]⁺.

¹H-NMR (300 MHz, DMSO-d₆): δ [ppm]=11.81 (s, 1H), 8.34-8.36 (m, 2H), 7.93-8.01 (m, 3H), 7.82-7.85 (m, 1H), 7.54-7.58 (m, 2H), 4.33 (q, 2H), 1.31 (t, 3H).

### Intermediate 81A

### Ethyl 3-cyclopropyl-6-(naphthalen-2-yl)-4-oxo-4,5-dihydropyrazolo[1,5-a]pyrazine-2-carboxylate

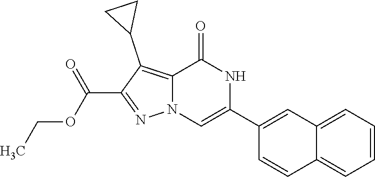 [SMILES: CCOC(=O)c1nn2cc(-c3ccc4ccccc4c3)[nH]c(=O)c2c1C1CC1]

To a solution of ethyl 3-iodo-6-(naphthalen-2-yl)-4-oxo-4,5-dihydropyrazolo[1,5-a]pyrazine-2-carboxylate (Intermediate 80A, 600 mg, 1.31 mmol) in THF (24.0 ml) under an argon atmosphere was added [(2-dicyclohexylphosphino-2′,6′-bis(N,N-dimethylamino)-1,1′-biphenyl)-2-(2′-amino-1,1′-biphenyl)]palladium(II) methanesulfonate (211 mg, 261 μmol), followed by dropwise addition of bromo(cyclopropyl)zinc solution (20.0 ml, 0.50 M in THF, 9.8 mmol) at 0° C. After complete addition, the cooling bath was removed, and the mixture was allowed to stir at RT for 3 days. Then, 1.0 M hydrochloric acid was added, and the THF was removed under reduced pressure. The remaining aqueous suspension was diluted with water, and the solid was filtered off, washed with MTBE and dried to afford the title compound (420 mg, 56% of theory, 65% purity) which was used in the next step without further purification.

LC/MS [Method 27]: Rₜ=1.42 min; MS (ESIneg): m/z=372 [M−H]⁻.

¹H-NMR (400 MHz, DMSO-d₆): δ [ppm]=11.62 (br. s, 1H), 8.38 (s, 1H), 8.25 (s, 1H), 8.03-7.95 (m, 3H), 7.87 (dd, 1H), 7.62-7.57 (m, 2H), 4.35 (q, 2H), 2.73-2.66 (m, 1H), 1.34 (t, 3H), 1.27-1.22 (m, 2H), 0.99-0.94 (m, 2H).

### Intermediate 82A

### 3-Cyclopropyl-6-(naphthalen-2-yl)-4-oxo-4,5-dihydropyrazolo[1,5-a]pyrazine-2-carboxylic acid

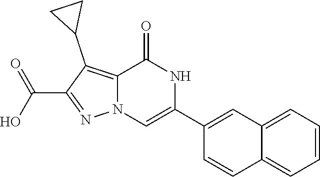 [SMILES: O=C(O)c1nn2cc(-c3ccc4ccccc4c3)[nH]c(=O)c2c1C1CC1]

A mixture of ethyl 3-cyclopropyl-6-(naphthalen-2-yl)-4-oxo-4,5-dihydropyrazolo[1,5-a]pyrazine-2-carboxylate (Intermediate 81A, 450 mg, 65% purity, 783 μmol) and lithium hydroxide (188 mg, 7.83 mmol) in ethanol (20 ml) and water (10 ml) was stirred overnight at RT. The ethanol was then removed under reduced pressure, and the aqueous suspension was diluted with water (30 ml). The mixture was acidified by addition of 1.0 M hydrochloric acid, and stirring was continued for 3 h at RT. The precipitate was filtered off, washed with water and dried under reduced pressure to afford the title compound which was used in the next step without further purification.

Yield: 400 mg (quant., 78% purity).

LC/MS [Method 3]: Rₜ=1.60 min; MS (ESIpos): m/z=344 [M+H]⁺.

¹H-NMR (400 MHz, DMSO-d₆): δ [ppm]=13.11 (br. s, 1H), 11.58 (s, 1H), 8.38 (s, 1H), 8.18 (d, 1H), 8.02 (d, 1H), 8.00-7.93 (m, 2H), 7.87 (dd, 1H), 7.64-7.56 (m, 2H), 2.80-2.72 (m, 1H), 1.34-1.22 (m, 2H), 1.00-0.88 (m, 2H).

### Intermediate 83A

### 2-[Ethyl(2-hydroxyethyl)amino]-1-phenylethanone

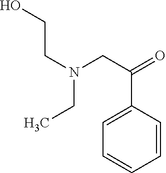

Potassium carbonate (10.4 g, 75.4 mmol) and 2-(ethylamino)ethanol (6.72 g, 75.4 mmol) were added to a solution of 2-bromo-1-phenylethanone (5.00 g, 25.1 mmol) in acetonitrile (150 ml), and the mixture was stirred at RT overnight. The solids were then removed by filtration, and the filtrate was concentrated under reduced pressure to afford the crude title compound which was used in the next step without further purification. Yield: 5.00 g (85% purity, 82% yield).

LC/MS [Method 3]: Rₜ=0.44 min; MS (ESIpos): m/z=208 [M+H]⁺.

¹H-NMR (400 MHz, DMSO-d₆): δ [ppm]=7.55-7.48 (m, 2H), 7.37-7.23 (m, 3H), 4.11-4.03 (m, 1H), 3.67-3.59 (m, 1H), 3.48-3.36 (m, 1H), 2.76-2.67 (m, 2H), 2.36-2.26 (m, 1H), 2.16-2.06 (m, 1H), 2.01 (d, 1H), 0.99 (t, 3H).

### Intermediate 84A

### 2-[Ethyl(2-hydroxyethyl)amino]-1-phenylethanone oxime

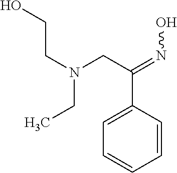 [SMILES: CCN(CCO)CC(=NO)c1ccccc1]

N,N-Diisopropylethylamine (7.8 ml, 45 mmol) and hydroxylammonium chloride (3.02 g, 43.4 mmol) were added to a solution of 2-[ethyl(2-hydroxyethyl)amino]-1-phenylethanone (Intermediate 83A, 4.50 g, 21.7 mmol) in ethanol (90 ml), and the mixture was stirred at 80° C. overnight. After cooling to RT, the mixture was evaporated to dryness, and the crude product thus obtained was used in the next step without further purification. Yield: 6.60 g (73% purity, 100% yield).

LC/MS [Method 27]: Rₜ=0.29 min; MS (ESIpos): m/z=223 [M+H]⁺.

### Intermediate 85A

### (rac)-2-[(2-Amino-2-phenylethyl)ethylamino]ethanol

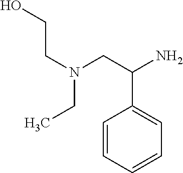

Palladium on activated carbon (157 mg, 10% Pd) was added to a solution of 2-[ethyl(2-hydroxyethyl)amino]-1-phenylethanone oxime (Intermediate 84A, 6.00 g, 73% purity, 19.7 mmol) in ethanol (100 ml), and the mixture was stirred under an atmosphere of hydrogen for 5 days. The catalyst was then filtered off, new palladium on activated carbon (157 mg, 10% Pd) was added, and stirring under an atmosphere of hydrogen was continued for 3 days. The catalyst was filtered off again, the solvent was removed under reduced pressure, and the residue was purified by preparative HPLC (Method P4) to afford the title compound. Yield: 627 mg (15% of theory, 95% purity).

LC/MS [Method 4]: Rₜ=1.26 min; MS (ESIpos): m/z=209 [M+H]⁺.

### Intermediate 86A

### Diethyl 4-methyl-1-[2-(2-naphthyl)-2-oxoethyl]-1H-pyrazole-3,5-dicarboxylate

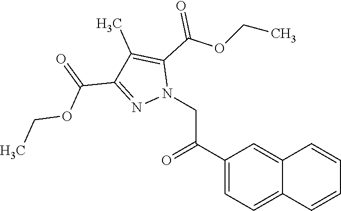 [SMILES: CCOC(=O)c1nn(CC(=O)c2ccc3ccccc3c2)c(C(=O)OCC)c1C]

To a solution of 2-bromo-1-(2-naphthyl)ethanone (4.79 g, 13.2 mmol) in acetone (70.0 ml) were added diethyl 4-methyl-1H-pyrazole-3,5-dicarboxylate (Intermediate 54A, 3.00 g, 13.2 mmol) and potassium carbonate (4.58 g, 33.2 mmol). The resulting mixture was stirred overnight at room temperature. The solids were then filtered off, and the filtrate was concentrated under reduced pressure to provide the crude product. This material was purified by flash-chromatography on silica gel (eluent: petroleum ether/ethyl acetate 9:1) to give 3.68 g (67% of theory, 96% purity) of the title compound.

LC/MS [Method 10]: Rₜ=1.23 min; MS (ESIpos): m/z=395 [M+H]⁺.

¹H-NMR (300 MHz, DMSO-d₆): δ [ppm]=8.83 (s, 1H), 7.99-8.18 (m, 4H), 7.65-7.76 (m, 2H), 6.34 (s, 2H), 4.32 (q, 2H), 4.18 (q, 2H), 2.53 (s, 3H), 1.31 (t, 3H), 1.15 (t, 3H).

### Intermediate 87A

### Ethyl 3-methyl-6-(2-naphthyl)-4-oxo-4,5-dihydropyrazolo[1,5-a]pyrazine-2-carboxylate

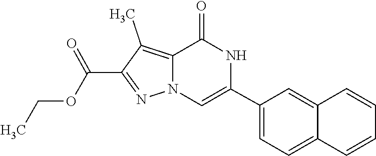 [SMILES: CCOC(=O)c1nn2cc(-c3ccc4ccccc4c3)[nH]c(=O)c2c1C]

To a solution of diethyl 4-methyl-1-[2-(2-naphthyl)-2-oxoethyl]-1H-pyrazole-3,5-dicarboxylate (Intermediate 86A, 3.68 g, 9.3 mmol) in acetic acid (50.0 ml) was added ammonium acetate (14.37 g, 189 mmol). The resulting mixture was stirred overnight at 110° C. After cooling to room temperature, the reaction mixture was poured into ice-water (80 ml). The solid was collected by filtration, washed with water and dried in vacuo to give 2.90 g (80% of theory, 90% purity) of the title compound.

LC/MS [Method 5]: Rₜ=1.15 min; MS (ESIpos): m/z=348 [M+H]⁺.

¹H-NMR (400 MHz, DMSO-d₆): δ [ppm]=11.68 (s, 1H), 8.38 (s, 1H), 8.27 (s, 1H), 7.96-8.04 (m, 3H), 7.86-7.89 (m, 1H), 7.58-7.61 (m, 2H), 4.35 (q, 2H), 2.67 (s, 3H), 1.34 (t, 3H).

### Intermediate 88A

### 3-Methyl-6-(2-naphthyl)-4-oxo-4,5-dihydropyrazolo[1,5-a]pyrazine-2-carboxylic acid

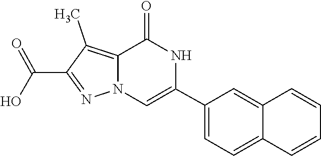 [SMILES: Cc1c(C(=O)O)nn2cc(-c3ccc4ccccc4c3)[nH]c(=O)c12]

To a solution of ethyl 3-methyl-6-(2-naphthyl)-4-oxo-4,5-dihydropyrazolo[1,5-a]pyrazine-2-carboxylate (Intermediate 87A, 2.80 g, 8.1 mmol) in ethanol (63.0 ml) and water (7.0 ml) was added sodium hydroxide (3.22 g, 80.6 mmol), and the mixture was stirred at RT for 4 h. The reaction mixture was then diluted with water (40.0 ml) and extracted with ethyl acetate. The organic layers were discarded, and the aqueous layer was adjusted to pH 3 with 1.0 M hydrochloric acid solution. The solid was collected by filtration and dried in vacuo to give 2.30 g (85% of theory, 96% purity) of the title compound.

LC/MS [Method 16]: Rₜ=0.86 min; MS (ESIpos): m/z=320 [M+H]⁺.

¹H-NMR (400 MHz, DMSO-d₆): δ [ppm]=13.09 (br. s, 1H), 11.64 (s, 1H), 8.38 (s, 1H), 8.21 (s, 1H), 7.97-8.04 (m, 3H), 7.87-7.89 (m, 1H), 7.57-7.62 (m, 2H), 2.66 (s, 3H).

### Intermediate 89A

### Diethyl 1-[2-(3,4-dimethylphenyl)-2-oxoethyl]-4-iodo-1H-pyrazole-3,5-dicarboxylate

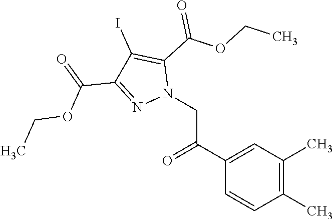 [SMILES: CCOC(=O)c1nn(CC(=O)c2ccc(C)c(C)c2)c(C(=O)OCC)c1I]

A mixture of diethyl 4-iodo-1H-pyrazole-3,5-dicarboxylate (Intermediate 78A, 4.00 g, 11.8 mmol), 2-bromo-1-(3,4-dimethylphenyl)ethanone (Intermediate 20A, 6.45 g, 50% purity, 14.2 mmol) and potassium carbonate (4.09 g, 29.6 mmol) in acetone (100 ml) was stirred overnight at RT. The solid material was then filtered off, and the filtrate was concentrated under reduced pressure to afford the crude product which was used in the next step without further purification. Yield: 5.30 g (51% of theory, 55% purity).

LC/MS [Method 27]: Rₜ=1.50 min; MS (ESIneg): m/z=483 [M−H]⁻.

### Intermediate 90A

### Ethyl 6-(3,4-dimethylphenyl)-3-iodo-4-oxo-4,5-dihydropyrazolo[1,5-a]pyrazine-2-carboxylate

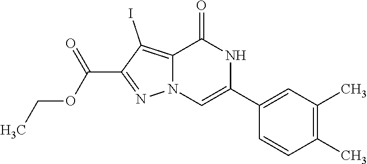 [SMILES: CCOC(=O)c1nn2cc(-c3ccc(C)c(C)c3)[nH]c(=O)c2c1I]

A mixture of diethyl 1-[2-(3,4-dimethylphenyl)-2-oxoethyl]-4-iodo-1H-pyrazole-3,5-dicarboxylate (Intermediate 89A, 7.40 g, 59% purity, 9.02 mmol) and ammonium acetate (27.8 g, 361 mmol) in acetic acid (180 ml) was heated to 110° C. and stirred at this temperature for 5 days. After cooling to RT, the mixture was poured in water (600 ml). The precipitate was filtered off, washed with ethyl acetate (300 ml) and MTBE (200 ml) and dried. The filtrate was concentrated to dryness and the residue was triturated with MTBE. The solid was filtered off and dried. Both crops of solid material were combined to afford 1.80 g (46% of theory) of the title compound.

LC/MS [Method 27]: Rₜ=1.35 min; MS (ESIpos): m/z=438 [M+H]⁺.

¹H-NMR (400 MHz, DMSO-d₆): δ [ppm]=11.63 (s, 1H), 8.14 (s, 1H), 7.56 (s, 1H), 7.47 (dd, 1H), 7.25 (d, 1H), 4.35 (q, 2H), 2.28 (s, 3H), 2.27 (s, 3H), 1.34 (t, 3H).

### Intermediate 91A

### Ethyl 3-cyclopropyl-6-(3,4-dimethylphenyl)-4-oxo-4,5-dihydropyrazolo[1,5-a]pyrazine-2-carboxylate

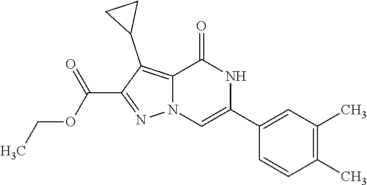 [SMILES: CCOC(=O)c1nn2cc(-c3ccc(C)c(C)c3)[nH]c(=O)c2c1C1CC1]

To a solution of ethyl 6-(3,4-dimethylphenyl)-3-iodo-4-oxo-4,5-dihydropyrazolo[1,5-a]pyrazine-2-carboxylate (Intermediate 90A, 500 mg, 1.14 mmol) in THF (20.0 ml) under an argon atmosphere was added [(2-dicyclohexylphosphino-2′,6′-bis(N,N-dimethylamino)-1,1-biphenyl)-2-(2′-amino-1,1′-biphenyl)]palladium(II) methanesulfonate (184 mg, 229 μmol), followed by dropwise addition of bromo(cyclopropyl)zinc solution (11.0 ml, 0.50 M in THF, 5.7 mmol) at 0° C. After complete addition, the cooling bath was removed, and the mixture was allowed to stir at RT for 3 days. Then, 1.0 M hydrochloric acid was added, and the mixture was directly subjected to purification by preparative HPLC (Method P5) to afford the title compound. Yield: 240 mg (55% of theory, 92% purity).

LC/MS [Method 3]: Rₜ=2.10 min; MS (ESIneg): m/z=350 [M−H]⁻.

¹H-NMR (400 MHz, DMSO-d₆): δ [ppm]=11.40 (s, 1H), 8.01 (s, 1H), 7.56 (s, 1H), 7.50-7.44 (m, 1H), 7.24 (d, 1H), 4.33 (q, 2H), 2.74-2.58 (m, 1H), 2.27 (s, 3H), 2.26 (s, 3H), 1.33 (t, 3H), 1.27-1.17 (m, 2H), 0.98-0.89 (m, 2H).

### Intermediate 92A

### 3-Cyclopropyl-6-(3,4-dimethylphenyl)-4-oxo-4,5-dihydropyrazolo[1,5-a]pyrazine-2-carboxylic acid

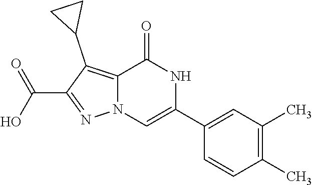 [SMILES: Cc1ccc(-c2cn3nc(C(=O)O)c(C4CC4)c3c(=O)[nH]2)cc1C]

A mixture of ethyl 3-cyclopropyl-6-(3,4-dimethylphenyl)-4-oxo-4,5-dihydropyrazolo[1,5-a]pyrazine-2-carboxylate (Intermediate 91A, 210 mg, 598 μmol) and lithium hydroxide (143 mg, 5.98 mmol) in ethanol (4.8 ml) and water (9.6 ml) was stirred at RT for 4 days. After complete conversion of the starting material, the mixture was acidified by addition of hydrochloric acid solution. The precipitate was filtered and dried under reduced pressure to afford the title compound which was used in the next step without further purification. Yield: 185 mg (67% of theory, 70% purity).

LC/MS [Method 3]: Rₜ=2.10 min; MS (ESIpos): m/z=324 [M+H]⁺.

¹H-NMR (400 MHz, DMSO-d₆): δ [ppm]=13.05 (br. s, 1H), 11.36 (s, 1H), 7.94 (d, 1H), 7.55 (s, 1H), 7.50-7.44 (m, 1H), 7.24 (d, 1H), 2.82-2.69 (m, 1H), 2.28 (s, 3H), 2.26 (s, 3H), 1.28-1.23 (m, 2H), 0.98-0.88 (m, 2H).

### Intermediate 93A

### N-Methoxy-N,4-dimethyl-3,4-dihydro-2H-1,4-benzoxazine-7-carboxamide

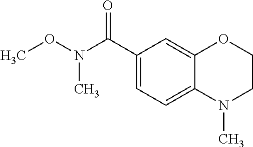 [SMILES: CON(C)C(=O)c1ccc2c(c1)OCCN2C]

To a solution of 4-methyl-3,4-dihydro-2H-1,4-benzoxazine-7-carboxylic acid (2.00 g, 10.35 mmol) in N,N-dimethylformamide (25.0 ml) was added 1-hydroxybenzotriazole (2.22 g, 14.49 mmol), 1-(3-dimethylaminopropyl)-3-ethylcarbodiimide hydrochloride (2.78 g, 14.49 mmol) and triethyl-amine (41.4 mmol, 5.8 ml). The reaction mixture was stirred at room temperature for 5 minutes before N-methoxymethanamine hydrochloride (1.21 g, 12.42 mmol) was added. The resulting mixture was stirred overnight at room temperature. The mixture was then poured into water (50 ml) and extracted with ethyl acetate (3×50 ml). The combined organic layers were dried over anhydrous sodium sulfate, filtered and concentrated under reduced pressure. The residue was purified by chromatography on silica gel (220 g silica gel; eluent: 0-40% ethyl acetate in petroleum ether) to give 2.20 g (88% of theory, 97% purity) of the title compound.

LC/MS [Method 6]: Rₜ=0.89 min; MS (ESIpos): m/z=237 [M+H]⁺.

¹H-NMR (400 MHz, CDCl₃): δ [ppm]=7.35-7.38 (m, 1H), 7.26-7.28 (m, 1H), 6.63-6.65 (m, 1H), 4.29-4.31 (m, 2H), 3.62 (s, 3H), 3.35-3.40 (m, 5H), 2.98 (s, 3H).

### Intermediate 94A

### 1-(4-Methyl-3,4-dihydro-2H-1,4-benzoxazin-7-yl)ethanone

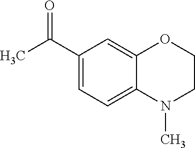

To a solution of N-methoxy-N,4-dimethyl-3,4-dihydro-2H-1,4-benzoxazine-7-carboxamide (Intermediate 93A, 2.20 g, 9.31 mmol) in THF (30.0 ml) was added methylmagnesium bromide (9.3 ml, 27.9 mmol, 3.0 M solution in diethyl ether) at −78° C. under nitrogen atmosphere. The reaction mixture was stirred at RT for 2 h. Then, the mixture was quenched by addition of brine (40 ml) and extracted with ethyl acetate (3×40 ml). The combined organic layers were washed with water (2×40 ml) and brine (2×40 ml) and dried over anhydrous sodium sulfate. Filtration, concentration and column chromatography on silica gel (220 g silica gel; eluent: petroleum ether/ethyl acetate 3:1) afforded 1.60 g (81% of theory, 90% purity) of the title compound. LC/MS [Method 28]: Rₜ=1.11 min; MS (ESIpos): m/z=192 [M+H]⁺. ¹H-NMR (300 MHz, CDCl₃): δ [ppm]=7.53-7.57 (m, 1H), 7.42 (s, 1H), 6.62-6.66 (m, 1H), 4.28-4.31 (m, 2H), 3.41-3.44 (m, 2H), 3.02 (s, 3H), 2.51 (s, 3H).

### Intermediate 95A

### 2-Bromo-1-(4-methyl-3,4-dihydro-2H-1,4-benzoxazin-7-yl)ethanone hydrobromide

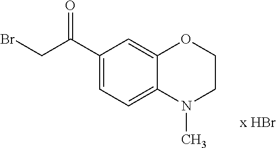

To a solution of 1-(4-methyl-3,4-dihydro-2H-1,4-benzoxazin-7-yl)ethanone (Intermediate 94A, 1.60 g, 7.53 mmol) in hydrobromic acid (20 ml, 33% solution in acetic acid) was added bromine (6.0 ml, 6.02 mmol, 1 M solution in acetic acid) dropwise. The reaction mixture was stirred at 60° C. for 2 h and then concentrated under reduce pressure to provide 1.90 g (54% of theory, 75% purity) of the title compound which was used in the next step without further purification.

LC/MS [Method 28]: Rₜ=1.24 min; MS (ESIpos): m/z=270/272 [M+H]⁺.

¹H-NMR (300 MHz, CDCl₃): δ [ppm]=7.62-7.67 (m, 2H), 7.55 (d, 1H), 4.59-4.66 (m, 2H), 4.40 (s, 2H), 3.68-3.71 (m, 2H), 3.29 (s, 3H).

### Intermediate 96A

### Diethyl 1-[2-(4-methyl-3,4-dihydro-2H-1,4-benzoxazin-7-yl)-2-oxoethyl]-1H-pyrazole-3,5-dicarboxylate

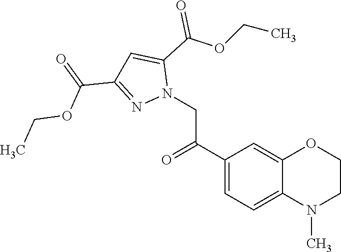 [SMILES: CCOC(=O)c1cc(C(=O)OCC)n(CC(=O)c2ccc3c(c2)OCCN3C)n1]

To a solution of 2-bromo-1-(4-methyl-3,4-dihydro-2H-1,4-benzoxazin-7-yl)ethanone hydrobromide (Intermediate 95A, 1.90 g, 4.05 mmol, 75% purity) in acetone (30.0 ml) were added diethyl 1H-pyrazole-3,5-dicarboxylate (0.78 g, 3.69 mmol) and potassium carbonate (1.02 g, 7.37 mmol). After stirring at room temperature overnight, the solids were filtered off, and the filtrate was concentrated under reduced pressure to provide the crude product. This material was purified by flash-chromatography on silica gel (120 g silica gel; eluent: petroleum ether/ethyl acetate 3:1) to give 1.60 g (99% of theory, 92% purity) of the title compound.

LC/MS [Method 29]: Rₜ=0.96 min; MS (ESIpos): m/z=402 [M+H]⁺.

¹H-NMR (400 MHz, CDCl₃): δ [ppm]=7.53-7.56 (m, 1H), 7.46 (s, 1H), 7.40 (s, 1H), 6.66 (d, 1H), 6.04 (s, 2H), 4.44 (q, 2H), 4.27-4.33 (m, 4H), 3.44-3.47 (m, 2H), 3.04 (s, 3H), 1.42 (t, 3H), 1.33 (t, 3H).

### Intermediate 97A

### Ethyl 6-(4-methyl-3,4-dihydro-2H-1,4-benzoxazin-7-yl)-4-oxo-4,5-dihydropyrazolo[1,5-a]-pyrazine-2-carboxylate

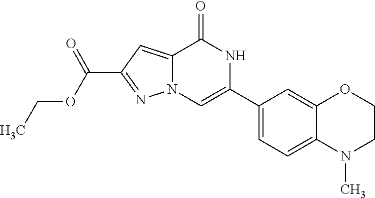 [SMILES: CCOC(=O)c1cc2c(=O)[nH]c(-c3ccc4c(c3)OCCN4C)cn2n1]

To a solution of diethyl 1-[2-(4-methyl-3,4-dihydro-2H-1,4-benzoxazin-7-yl)-2-oxoethyl]-1H-pyrazole-3,5-dicarboxylate (Intermediate 96A, 800 mg, 1.8 mmol) in acetic acid (20.0 ml) was added ammonium acetate (2.84 g, 36.7 mmol), and the reaction mixture was stirred at 130° C. for 5 h under an atmosphere of nitrogen. Then, the reaction mixture was cooled to room temperature and poured into water (50.0 ml). The crude product was collected by filtration and washed with water. The solid thus obtained was purified by column chromatography on silica gel (80 g silica gel; eluent: petroleum ether/ethyl acetate 1:5) to give 410 mg (60% of theory, 96% purity) of the title compound.

LC/MS [Method 6]: Rₜ=1.00 min; MS (ESIpos): m/z=355 [M+H]⁺.

¹H-NMR (300 MHz, DMSO-d₆): δ [ppm]=11.58 (s, 1H), 8.04 (s, 1H), 7.38 (s, 1H), 7.22-7.25 (m, 1H), 7.12 (s, 1H), 6.77 (d, 1H), 4.35 (q, 2H), 4.25-4.28 (m, 2H), 3.31-3.33 (m, 2H), 2.91 (s, 3H), 1.34 (t, 3H).

### Intermediate 98A

### 6-(4-Methyl-3,4-dihydro-2H-1,4-benzoxazin-7-yl)-4-oxo-4,5-dihydropyrazolo[1,5-a]pyrazine-2-carboxylic acid

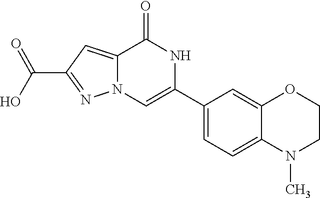 [SMILES: CN1CCOc2cc(-c3cn4nc(C(=O)O)cc4c(=O)[nH]3)ccc21]

To a mixture of ethyl 6-(4-methyl-3,4-dihydro-2H-1,4-benzoxazin-7-yl)-4-oxo-4,5-dihydropyrazolo[1,5-a]pyrazine-2-carboxylate (Intermediate 97A, 410 mg, 1.15 mmol) and ethanol (10.0 ml) was added a solution of sodium hydroxide (463 mg, 11.6 mmol) in water (3.0 ml). The reaction mixture was stirred at RT for 3 h. The mixture was then diluted with water (10.0 ml), and the ethanol was evaporated off. The remaining mixture was acidified with 1.0 M hydrochloric acid to pH 3. The precipitate was collected by filtration, washed with water and dried to afford 334 mg (83% of theory, 94% purity) of the title compound.

LC/MS [Method 2]: Rₜ=0.98 min; MS (ESIpos): m/z=327 [M+H]⁺.

¹H-NMR (300 MHz, DMSO-d₆): δ [ppm]=13.25 (br. s, 1H), 11.53 (s, 1H), 7.99 (s, 1H), 7.31 (s, 1H), 7.21-7.25 (m, 1H), 7.12 (s, 1H), 6.76 (d, 1H), 4.25 (t, 2H), 3.30-3.32 (m, 2H), 2.90 (s, 3H).

### Intermediate 99A

### 2-Bromo-1-(2,3-dihydro-1,4-benzodioxin-6-yl)ethanone

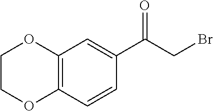 [SMILES: O=C(CBr)c1ccc2c(c1)OCCO2]

A mixture of 1-(2,3-dihydro-1,4-benzodioxin-6-yl)ethanone (25.0 g, 140 mmol) and phenyltrimethylammonium tribromide (52.7 g, 140 mmol) in dichloromethane (300 ml) was stirred at RT overnight. The ammonium salts were filtered off, and the filter cake was washed with ethyl acetate. The filtrate was concentrated under reduced pressure, and the solid product thus obtained was used in the next step without further purification. Yield: 34.35 g (95% of theory).

¹H-NMR (400 MHz, CDCl₃): δ [ppm]=7.50-7.54 (m, 2H), 6.91-6.95 (m, 1H), 4.37 (s, 2H), 4.27-4.35 (m, 4H).

### Intermediate 100A

### Diethyl 1-[2-(2,3-dihydro-1,4-benzodioxin-5-yl)-2-oxoethyl]-1H-pyrazole-3,5-dicarboxylate

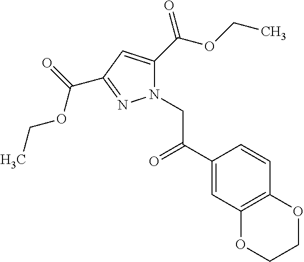 [SMILES: CCOC(=O)c1cc(C(=O)OCC)n(CC(=O)c2ccc3c(c2)OCCO3)n1]

A mixture of diethyl 1H-pyrazole-3,5-dicarboxylate (5.00 g, 23.6 mmol), 2-bromo-1-(2,3-dihydro-1,4-benzodioxin-6-yl)ethanone (Intermediate 99A, 6.66 g, 25.92 mmol) and potassium carbonate (3.58 g, 25.9 mmol) in acetone (100 ml) was stirred at RT overnight. After filtering off the solids, the filtrate was evaporated, and the residue was partitioned between dichloromethane and water. The organic phase was washed with water and brine, dried over sodium sulfate and evaporated to give the title compound. Yield: 8.00 g (87% of theory).

¹H-NMR (400 MHz, DMSO-d₆): δ [ppm]=7.56-7.59 (m, 2H), 7.34 (s, 1H), 7.05 (d, 1H), 6.15 (s, 2H), 4.28-4.37 (m, 6H), 4.20 (q, 2H), 1.30 (t, 3H), 1.19 (t, 3H).

### Intermediate 101A

### Ethyl 6-(2,3-dihydro-1,4-benzodioxin-6-yl)-4-oxo-4,5-dihydropyrazolo[1,5-a]pyrazine-2-carboxylate

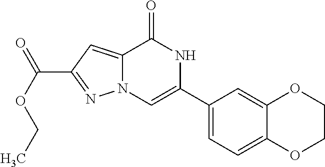 [SMILES: CCOC(=O)c1cc2c(=O)[nH]c(-c3ccc4c(c3)OCCO4)cn2n1]

To a solution of diethyl 1-[2-(2,3-dihydro-1,4-benzodioxin-6-yl)-2-oxoethyl]-1H-pyrazole-3,5-dicarboxylate (Intermediate 100A, 9.70 g, 25.0 mmol) in acetic acid (100 ml) was added ammonium acetate (38.5 g, 500 mmol), and the mixture was stirred at 110° C. overnight. After cooling to RT, the solution was poured into ice-water. The precipitate was filtered off, washed with water and dried to afford the title compound which was used in the next step without further purification.

Yield: 8.30 g (crude product, contained 6-(2,3-dihydro-1,4-benzodioxin-6-yl)-4-oxo-4,5-dihydro-pyrazolo[1,5-a]pyrazine-2-carboxylic acid).

¹H-NMR (400 MHz, DMSO-d₆): δ [ppm]=11.65 (s, 1H), 8.10 (s, 1H), 7.39 (s, 1H), 7.23-7.34 (m, 2H), 6.96 (d, 1H), 4.29-4.36 (m, 6H), 1.33 (t, 3H).

### Intermediate 102A

### 6-(2,3-Dihydro-1,4-benzodioxin-6-yl)-4-oxo-4,5-dihydropyrazolo[1,5-a]pyrazine-2-carboxylic acid

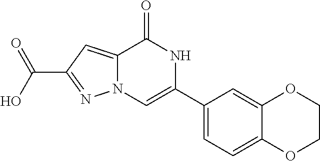 [SMILES: O=C(O)c1cc2c(=O)[nH]c(-c3ccc4c(c3)OCCO4)cn2n1]

A 250 ml round-bottom flask was charged with ethyl 6-(2,3-dihydro-1,4-benzodioxin-6-yl)-4-oxo-4,5-dihydropyrazolo[1,5-a]pyrazine-2-carboxylate (Intermediate 101A, 8.30 g, 24.3 mmol) and ethanol (40 ml). An aqueous solution of sodium hydroxide (20 ml, 3.0 M) was added, and the mixture was stirred at RT for 4 h. Then, diluted hydrochloric acid (3.0 M) was added until pH 6 was reached, and the precipitate was collected by filtration and washed with ethanol containing 5% N,N-dimethylformamide. The filter cake was dried to afford the title compound. Yield: 4.30 g (55% of theory, 97% purity).

LC/MS [Method 2]: Rₜ=0.94 min; MS (ESIpos): m/z=314 [M+H]⁺.

¹H-NMR (400 MHz, DMSO-d₆): δ [ppm]=13.26 (br. s, 1H), 11.65 (s, 1H), 8.06 (s, 1H), 7.31-7.33 (m, 2H), 7.24 (d, 1H), 6.97 (d, 1H), 4.20-4.30 (m, 4H).

### Intermediate 103A

### tert.-Butyl 3-amino-3-(4-fluorophenyl)azetidine-1-carboxylate

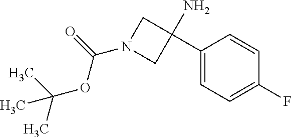 [SMILES: CC(C)(C)OC(=O)N1CC(N)(c2ccc(F)cc2)C1]

To a solution of tert.-butyl 3-azido-3-(4-fluorophenyl)azetidine-1-carboxylate (Intermediate 45A, 4.00 g, 13.7 mmol) in methanol (100 ml) was added palladium on carbon (600 mg, 10% palladium on carbon dry loading, 50% wet with water). The resulting mixture was vigorously stirred at ambient temperature for 24 h under a hydrogen atmosphere (2-3 atm) and then filtered through Celite. The filtrate was concentrated under reduced pressure to afford the title compound. Yield: 3.04 g (73% of theory, 88% purity).

LC/MS [Method 22]: Rₜ=1.74 min; MS (ESIpos): m/z=211 [M+H−C₄H₈]⁺.

¹H-NMR (300 MHz, DMSO-d₆): δ [ppm]=7.50-7.55 (m, 2H), 7.15-7.21 (m, 2H), 4.01 (d, 2H), 3.91 (d, 2H), 3.01 (br. s, 2H), 1.38 (s, 9H).

### Intermediate 104A

### tert.-Butyl 3-hydroxy-3-(4-methoxyphenyl)azetidine-1-carboxylate

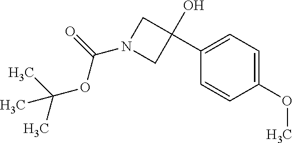 [SMILES: COc1ccc(C2(O)CN(C(=O)OC(C)(C)C)C2)cc1]

To a solution of tert.-butyl 3-oxoazetidine-1-carboxylate (20.00 g, 117 mmol) in THF (200 ml) was added (4-methoxyphenyl)magnesium bromide (140 ml, 140 mmol, 1 M solution in THF). The reaction mixture was stirred at 0° C. for 3 h under an atmosphere of nitrogen. Then, a saturated aqueous solution of ammonium chloride (200 ml) was added, and the mixture was extracted with dichloromethane (2×400 ml). The combined organic layers were washed with water (2×400 ml) and brine (2×400 ml) and dried over anhydrous sodium sulfate. Filtration, concentration and chromatography on silica gel (eluent: petroleum ether/ethyl acetate 3:1) gave the title compound. Yield: 27.10 g (79% of theory, 95% purity).

LC/MS [Method 8]: Rₜ=1.14 min; MS (ESIpos): m/z=559 [2M+H]⁺.

¹H-NMR (300 MHz, CDCl₃): δ [ppm]=1.50 (s, 9H), 3.84 (s, 3H), 4.17 (d, 2H), 4.26 (d, 2H), 6.94 (d, 2H), 7.42 (d, 2H).

### Intermediate 105A

### tert.-Butyl 3-azido-3-(4-methoxyphenyl)azetidine-1-carboxylate

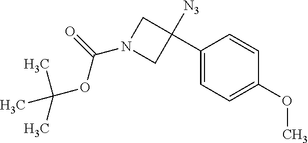 [SMILES: COc1ccc(C2(N=[N+]=[N-])CN(C(=O)OC(C)(C)C)C2)cc1]

To a solution of tert.-butyl 3-hydroxy-3-(4-methoxyphenyl)azetidine-1-carboxylate (Intermediate 104A, 10.0 g, 35.8 mmol) in THF (200 ml) were added triphenylphosphine (11.7 g, 44.8 mmol), diphenyl phosphoroazidate (10.0 ml, 46.5 mmol) and diisopropyl azodicarboxylate (9.16 ml, 46.5 mmol) under nitrogen atmosphere. After stirring at room temperature for 16 hours, the reaction mixture was quenched with water and extracted with ethyl acetate. The combined organic layers were washed with water, dried over anhydrous sodium sulfate and filtered. The filtrate was concentrated under reduced pressure, and the residue was purified by flash-chromatography on silica gel (eluent: petroleum ether/ethyl acetate 15:1→4:1) to give the title compound. Yield: 7.80 g (71% of theory).

¹H-NMR (400 MHz, DMSO-d₆): δ [ppm]=7.39 (d, 2H), 7.01 (d, 2H), 4.33 (d, 2H), 4.16 (d, 2H), 3.79 (s, 3H), 1.39 (s, 9H).

### Intermediate 106A

### tert.-Butyl 3-amino-3-(4-methoxyphenyl)azetidine-1-carboxylate

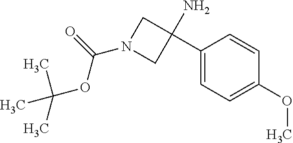 [SMILES: COc1ccc(C2(N)CN(C(=O)OC(C)(C)C)C2)cc1]

tert.-Butyl 3-azido-3-(4-methoxyphenyl)azetidine-1-carboxylate (Intermediate 105A, 4.8 g, 15.8 mmol) was dissolved in methanol (50.0 ml), and 10% palladium on active carbon (800 mg) was added. The mixture was stirred under a hydrogen atmosphere (2-3 atm) at room temperature overnight. Then, the catalyst was filtered off, and the solvent was removed under reduced pressure. The residue was purified by flash-chromatography on silica gel (eluent: petroleum ether/0-100% ethyl acetate) to afford the title compound. Yield: 3.16 g (67% of theory, 93% purity).

LC/MS [Method 1]: Rₜ=1.43 min; MS (ESIpos): m/z=557 [2M+H]⁺.

¹H-NMR (300 MHz, DMSO-d₆): δ [ppm]=7.40 (d, 2H), 6.91 (d, 2H), 3.99 (d, 2H), 3.88 (d, 2H), 3.74 (s, 3H), 2.45 (s, 2H), 1.39 (s, 9H).

### Intermediate 107A

### Ethyl 3-cyano-6-(3,4-dimethylphenyl)-4-oxo-4,5-dihydropyrazolo[1,5-a]pyrazine-2-carboxylate

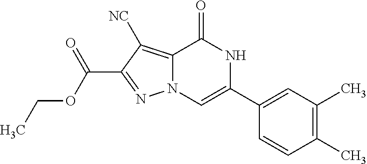 [SMILES: CCOC(=O)c1nn2cc(-c3ccc(C)c(C)c3)[nH]c(=O)c2c1C#N]

A mixture of ethyl 6-(3,4-dimethylphenyl)-3-iodo-4-oxo-4,5-dihydropyrazolo[1,5-a]pyrazine-2-carboxylate (Intermediate 90A, 900 mg, 2.06 mmol) and copper(I) cyanide (369 mg, 4.12 mmol) in DMF (18.7 ml) was stirred at 150° C. for 90 min. After cooling to RT, the mixture was poured into water (150 ml), and the precipitate was collected by filtration, washed with water and dried to afford the title compound. Yield: 740 mg (quant., 94% purity).

LC/MS [Method 3]: Rₜ=1.85 min; MS (ESIneg): m/z=335 [M−H]⁻.

### Intermediate 108A

### 3-Cyano-6-(3,4-dimethylphenyl)-4-oxo-4,5-dihydropyrazolo[1,5-a]pyrazine-2-carboxylic acid

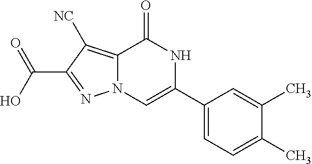 [SMILES: Cc1ccc(-c2cn3nc(C(=O)O)c(C#N)c3c(=O)[nH]2)cc1C]

A mixture of ethyl 3-cyano-6-(3,4-dimethylphenyl)-4-oxo-4,5-dihydropyrazolo[1,5-a]pyrazine-2-carboxylate (Intermediate 107A, 740 mg, 94% purity, 2.07 mmol) and lithium hydroxide (495 mg, 20.7 mmol) in ethanol (8.3 ml) and water (4.2 ml) was stirred at RT for 4 h. The ethanol was removed under reduced pressure, and the resulting suspension was diluted with water to a final volume of 80 ml. Then, 1.0 M hydrochloric acid was added until pH 2 was reached, and the solid was collected by filtration, washed with water and dried to afford the title compound. Yield: 410 mg (62% of theory).

LC/MS [Method 7]: Rₜ=0.69 min; MS (ESIneg): m/z=307 [M−H]⁻.

### Intermediate 109A

### 3-(4-Methoxyphenyl)-1-methylazetidin-3-amine

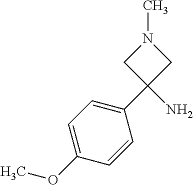

To a solution of tent.-butyl 3-azido-3-(4-methoxyphenyl)azetidine-1-carboxylate (Intermediate 105A, 4.80 g, 15.8 mmol) in THF (80 ml) was added lithium aluminium hydride (2.39 g, 63.1 mmol) in portions at 0° C. The reaction was stirred for 20 min at room temperature and for 2 h at 60° C. Then, the reaction mixture was cooled to −20° C., and sodium sulfate decahydrate was added until no further exothermic reaction was observed. After filtering off the solids, the filtrate was evaporated, and the residue was purified by flash-chromatography on silica gel [eluent: 0-5% methanol in dichloromethane (+0.5% triethylamine)] to give the title compound. Yield: 1.67 g (50% of theory, 90% purity).

LC/MS [Method 1]: Rₜ=0.58 min; MS (ESIpos): m/z=193 [M+H]⁺.

¹H-NMR (300 MHz, DMSO-d₆): δ [ppm]=7.45 (d, 2H), 6.89 (d, 2H), 3.73 (s, 3H), 3.53 (d, 2H), 3.20 (d, 2H), 2.31 (s, 3H).

### Intermediate 110A

### Ethyl 3-cyano-6-(naphthalen-2-yl)-4-oxo-4,5-dihydropyrazolo[1,5-a]pyrazine-2-carboxylate

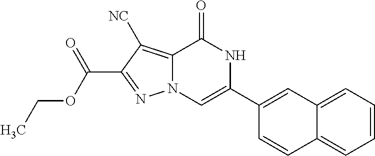 [SMILES: CCOC(=O)c1nn2cc(-c3ccc4ccccc4c3)[nH]c(=O)c2c1C#N]

A mixture of ethyl 3-iodo-6-(naphthalen-2-yl)-4-oxo-4,5-dihydropyrazolo[1,5-a]pyrazine-2-carboxylate (Intermediate 80A, 2.50 g, 5.44 mmol) and copper(I) cyanide (975 mg, 10.9 mmol) in DMF (50.0 ml) was stirred at 150° C. for 90 min. After cooling to RT, the mixture was poured into water (300 ml), and the precipitate was collected by filtration, washed with water and dried to afford the title compound. Yield: 2.00 g (94% of theory, 92% purity).

LC/MS [Method 3]: Rₜ=1.84 min; MS (ESIneg): m/z=357 [M−H]⁻.

### Intermediate 111A

### 3-Cyano-6-(naphthalen-2-yl)-4-oxo-4,5-dihydropyrazolo[1,5-a]pyrazine-2-carboxylic acid

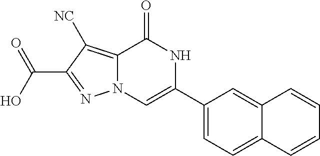 [SMILES: N#Cc1c(C(=O)O)nn2cc(-c3ccc4ccccc4c3)[nH]c(=O)c12]

A mixture of ethyl 3-cyano-6-(naphthalen-2-yl)-4-oxo-4,5-dihydropyrazolo[1,5-a]pyrazine-2-carboxylate (Intermediate 110A, 1.95 g, 5.44 mmol) and lithium hydroxide (1.30 g, 54.4 mmol) in ethanol (22 ml) and water (11 ml) was stirred at RT for 4 h. The ethanol was removed under reduced pressure, and the resulting suspension was diluted with water to a final volume of 100 ml. Then, 1.0 M hydrochloric acid was added until pH 2 was reached, and the solid was collected by filtration, washed with water and dried to afford the title compound. Yield: 1.75 g (97% of theory).

LC/MS [Method 3]: Rₜ=1.22 min; MS (ESIneg): m/z=329 [M−H]⁻.

### Intermediate 112A

### N-Methoxy-N-methylquinoline-7-carboxamide

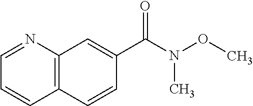 [SMILES: CON(C)C(=O)c1ccc2cccnc2c1]

N-Methoxymethanamine hydrochloride (4.96 g, 50.8 mmol) and triethylamine (18.70 g, 185 mmol) were added to a suspension of quinoline-7-carboxylic acid (8.00 g, 46.2 mmol), 1-(3-dimethylaminopropyl)-3-ethylcarbodiimide hydrochloride (9.74 g, 50.8 mmol) and 1-hydroxybenzotriazole (7.78 g, 50.8 mmol) in dichloromethane (160 ml). The mixture was stirred at ambient temperature overnight. The reaction mixture was then diluted with dichloromethane (100 ml) and washed with aqueous sodium carbonate solution (2×100 ml) and water (3×100 ml). The aqueous layers were extracted with dichloromethane (3×100 ml), and the combined organic layers were dried over sodium sulfate, filtered and concentrated to afford the title compound. Yield: 7.66 g (69% of theory, 90% purity).

LC/MS [Method 6]: Rₜ=0.64 min; MS (ESIpos): m/z=217 [M+H]⁺.

¹H-NMR (400 MHz, CDCl₃): δ [ppm]=8.95-8.97 (m, 1H), 8.43 (s, 1H), 8.18 (d, 1H), 7.79-7.86 (m, 2H), 7.44-7.47 (m, 1H), 3.58 (s, 3H), 3.42 (s, 3H).

### Intermediate 113A

### 1-(Quinolin-7-yl)ethanone

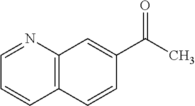

A solution of N-methoxy-N-methylquinoline-7-carboxamide (Intermediate 112A, 7.66 g, 31.9 mmol, 90% purity) in THF (150 ml) was degassed with nitrogen three times. Subsequently, methylmagnesium bromide (38.3 ml, 38.3 mmol, 1.0 M solution in THF) was added slowly at 0° C. After complete addition, the reaction mixture was stirred at room temperature for 2 h. Then, water (100 ml) was added, and the mixture was extracted with ethyl acetate (5×100 ml). The combined organic layers were dried over anhydrous sodium sulfate, filtered and concentrated to give the title compound. Yield: 4.30 g (77% of theory, 97% purity).

LC/MS [Method 6]: Rₜ=0.69 min; MS (ESIpos): m/z=172 [M+H]⁺.

¹H-NMR (400 MHz, CDCl₃): δ [ppm]=9.01-9.02 (m, 1H), 8.71 (s, 1H), 8.23 (d, 1H), 8.14 (dd, 1H), 7.90 (d, 1H), 7.51-7.54 (m, 1H), 2.77 (s, 3H).

### Intermediate 114A

### 2-Bromo-1-(quinolin-7-yl)ethanone hydrobromide

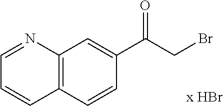 [SMILES: O=C(CBr)c1ccc2cccnc2c1]

A mixture of 1-(quinolin-7-yl)ethanone (Intermediate 113A, 3.30 g, 18.7 mmol, 97% purity), bromine (5.99 g, 37.5 mmol) and hydrobromic acid (3.47 g, 20.6 mmol, 48% solution in water) in dichloromethane (150 ml) was stirred at room temperature for two days. The precipitate was collected by filtration, washed with dichloromethane and dried to afford the crude 2-bromo-1-(quinolin-7-yl)ethanone hydrobromide (9.00 g, 71% purity according to LC/MS) which was used in the next step without further purification.

LC/MS [Method 6]: Rₜ=0.87 min; MS (ESIpos): m/z=250 [M+H]⁺.

### Intermediate 115A

### Diethyl 1-[2-oxo-2-(quinolin-7-yl)ethyl]-1H-pyrazole-3,5-dicarboxylate

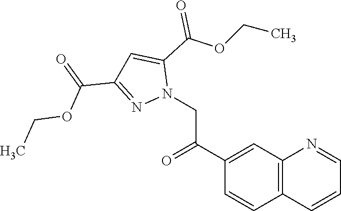 [SMILES: CCOC(=O)c1cc(C(=O)OCC)n(CC(=O)c2ccc3cccnc3c2)n1]

A mixture of diethyl 1H-pyrazole-3,5-dicarboxylate (3.00 g, 14.1 mmol), 2-bromo-1-(quinolin-7-yl)ethanone hydrobromide (Intermediate 114A, 6.59 g, 14.1 mmol, 71% purity) and potassium carbonate (2.15 g, 15.6 mmol) in acetone (40.0 ml) was stirred at room temperature overnight. After filtering off the solids, the filtrate was evaporated, and the residue was partitioned between dichloromethane and water. The organic phase was washed with water and brine, dried over sodium sulfate and evaporated to give the title compound. Yield: 3.42 g (63% of theory).

LC/MS [Method 5]: Rₜ=0.99 min; MS (ESIpos): m/z=382 [M+H]⁺.

¹H-NMR (400 MHz, DMSO-d₆): δ [ppm]=9.07-9.09 (m, 1H), 8.84 (s, 1H), 8.51 (d, 1H), 8.18 (d, 1H), 8.10 (d, 1H), 7.70-7.73 (m, 1H), 7.38 (s, 1H), 6.46 (s, 2H), 4.33 (q, 2H), 4.20 (q, 2H), 1.32 (t, 3H), 1.16 (t, 3H).

### Intermediate 116A

### Ethyl 4-oxo-6-(quinolin-7-yl)-4,5-dihydropyrazolo[1,5-a]pyrazine-2-carboxylate

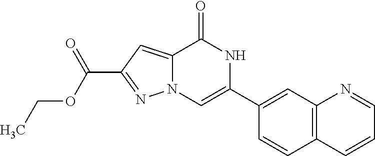 [SMILES: CCOC(=O)c1cc2c(=O)[nH]c(-c3ccc4cccnc4c3)cn2n1]

Ammonium acetate (13.8 g, 179 mmol) was added to a solution of diethyl 1-[2-oxo-2-(quinolin-7-yl)ethyl]-1H-pyrazole-3,5-dicarboxylate (Intermediate 115A, 3.42 g, 8.97 mmol) in acetic acid (50 ml), and the mixture was stirred at 110° C. overnight. After cooling to RT, the solution was poured into ice-water. The solid was collected by filtration, washed with water and dried in air to give the title compound. Yield: 2.80 g (84% of theory, 90% purity).

LC/MS [Method 6]: Rₜ=0.81 min; MS (ESIpos): m/z=335 [M+H]⁺.

¹H-NMR (400 MHz, DMSO-d₆): δ [ppm]=11.79 (s, 1H), 8.97-8.99 (m, 1H), 8.41-8.46 (m, 3H), 8.10 (d, 1H), 7.97 (d, 1H), 7.58-7.61 (m, 1H), 7.45 (s, 1H), 4.36 (q, 2H), 1.34 (t, 3H).

### Intermediate 117A

### 4-Oxo-6-(quinolin-7-yl)-4,5-dihydropyrazolo[1,5-a]pyrazine-2-carboxylic acid

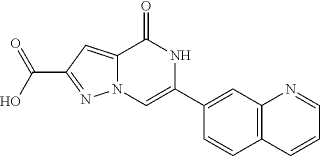 [SMILES: O=C(O)c1cc2c(=O)[nH]c(-c3ccc4cccnc4c3)cn2n1]

A 100 ml round-bottom flask was charged with ethyl 4-oxo-6-(quinolin-7-yl)-4,5-dihydropyrazolo-[1,5-a]pyrazine-2-carboxylate (Intermediate 116A, 2.80 g, 7.54 mmol, 90% purity) and ethanol (20 ml). An aqueous solution of sodium hydroxide (15.0 ml, 3.0 M) was added, and the mixture was stirred for 4 h at room temperature. Hydrochloric acid (3.0 M) was then added until pH 6 was reached. The precipitate was collected by filtration, washed with water and dried to afford the title compound. Yield: 1.86 g (79% of theory, 98% purity).

LC/MS [Method 30]: Rₜ=0.69 min; MS (ESIpos): m/z=307 [M+H]⁺.

¹H-NMR (300 MHz, DMSO-d₆): δ [ppm]=12.11 (s, 1H), 9.12 (d, 1H), 8.72-8.75 (m, 1H), 8.51 (s, 1H), 8.43 (s, 1H), 8.24 (d, 1H), 8.10 (d, 1H), 7.79-7.83 (m, 1H), 7.39 (s, 1H).

### Intermediate 118A

### Diethyl 4-chloro-1H-pyrazole-3,5-dicarboxylate

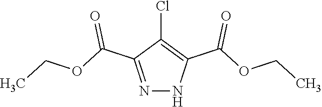 [SMILES: CCOC(=O)c1n[nH]c(C(=O)OCC)c1Cl]

To a solution of diethyl 1H-pyrazole-3,5-dicarboxylate (20.0 g, 94.2 mmol) in acetic acid (360 ml) was added dropwise sodium hypochlorite solution (220 ml, 9% in water). After stirring for 6 h at room temperature, the reaction mixture was concentrated under reduced pressure to remove acetic acid, and the remaining mixture was diluted with water (1000 ml). 1.0 M hydrochloric acid was added until pH 4 was reached, and the mixture was extracted with ethyl acetate (3×700 ml). The combined organic layers were washed with water and brine, dried over anhydrous sodium sulfate, filtered and concentrated under reduced pressure. Purification of the residue by flash-chromatography on silica gel (eluent: ethyl acetate/petroleum ether 1:1) afforded the title compound. Yield: 20.0 g (76% of theory, 88% purity).

¹H-NMR (300 MHz, DMSO-d₆): δ [ppm]=14.93 (br. s, 1H), 4.43-4.30 (m, 4H), 1.39-1.30 (m, 6H).

### Intermediate 119A

### Diethyl 4-chloro-1-[2-(3,4-dimethylphenyl)-2-oxoethyl]-1H-pyrazole-3,5-dicarboxylate

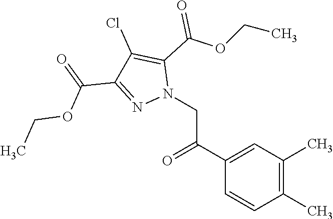 [SMILES: CCOC(=O)c1nn(CC(=O)c2ccc(C)c(C)c2)c(C(=O)OCC)c1Cl]

To a solution of diethyl 4-chloro-1H-pyrazole-3,5-dicarboxylate (Intermediate 118A, 9.10 g, 36.89 mmol) in acetone (200 ml) were added 2-bromo-1-(3,4-dimethylphenyl)ethanone (Intermediate 20A, 11.10 g, 36.89 mmol, 75% purity) and potassium carbonate (7.65 g, 55.34 mmol). The mixture was stirred at RT overnight. After filtering off the solids, the filter cake was washed with ethyl acetate, and the filtrate was concentrated under reduced pressure. The residue was taken up in ethyl acetate/petroleum ether (132 ml, 1:10) and stirred for 30 minutes. The solid was collected to by filtration and dried to give the title compound (11.00 g, 64% of theory, 84% purity).

¹H-NMR (400 MHz, DMSO-d₆): δ [ppm]=7.80 (s, 1H), 7.77 (dd, 1H), 7.35 (d, 1H), 6.19 (s, 2H), 4.41-4.26 (m, 2H), 4.20 (q, 2H), 2.31 (s, 3H), 2.30 (s, 3H), 1.29 (t, 3H), 1.12 (t, 3H).

### Intermediate 120A

### Ethyl 3-chloro-6-(3,4-dimethylphenyl)-4-oxo-4,5-dihydropyrazolo[1,5-a]pyrazine-2-carboxylate

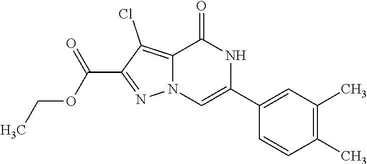 [SMILES: CCOC(=O)c1nn2cc(-c3ccc(C)c(C)c3)[nH]c(=O)c2c1Cl]

Ammonium acetate (31.43 g, 274.15 mmol) was added to a solution of diethyl 4-chloro-1-[2-(3,4-dimethylphenyl)-2-oxoethyl]-1 H-pyrazole-3,5-dicarboxylate (Intermediate 119A, 6.40 g, 13.71 mmol, 84% purity) in acetic acid (80 ml), and the mixture was stirred at 120° C. overnight. After cooling to RT, the reaction mixture was poured into water (150 ml), and the precipitate was filtered off. The filter cake was washed with water and dried in air to give 5.02 g (95% of theory, 90% purity) of the title compound.

¹H-NMR (300 MHz, DMSO-d₆): δ [ppm]=11.73 (br. s, 1H), 8.12 (s, 1H), 7.61-7.53 (m, 1H), 7.48 (dd, 1H), 7.26 (d, 1H), 4.37 (q, 2H), 2.29 (s, 3H), 2.28 (s, 3H), 1.35 (t, 3H).

### Intermediate 121A

### 3-Chloro-6-(3,4-dimethylphenyl)-4-oxo-4,5-dihydropyrazolo[1,5-a]pyrazine-2-carboxylic acid

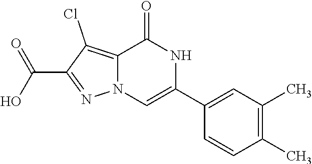 [SMILES: Cc1ccc(-c2cn3nc(C(=O)O)c(Cl)c3c(=O)[nH]2)cc1C]

To a solution of ethyl 3-chloro-6-(3,4-dimethylphenyl)-4-oxo-4,5-dihydropyrazolo[1,5-a]pyrazine-2-carboxylate (Intermediate 120A, 4.80 g, 12.49 mmol, 90% purity) in ethanol (200 ml) was added a solution of sodium hydroxide (5.35 g, 124.93 mmol) in water (40 ml). After stirring at room temperature for 3 h, 2.0 M hydrochloric acid was added to the reaction mixture to adjust the pH value to 1. The precipitate was collected by filtration, washed with water and dried in air to afford 4.13 g (94% of theory, 90% purity) of the title compound.

¹H-NMR (300 MHz, DMSO-d₆): δ [ppm]=13.50 (br. s, 1H), 11.68 (s, 1H), 8.06 (s, 1H), 7.56 (s, 1H), 7.48 (dd, 1H), 7.25 (d, 1H), 2.28 (s, 3H), 2.27 (br. s, 3H).

### Intermediate 122A

### N-Methoxy-N,4-dimethyl-3-(trifluoromethyl)benzamide

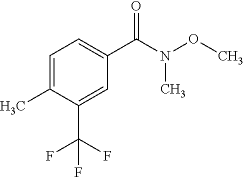

To a suspension of 4-methyl-3-(trifluoromethyl)benzoic acid (10.0 g, 49.0 mmol), 1-(3-dimethylaminopropyl)-3-ethylcarbodiimide hydrochloride (10.33 g, 53.9 mmol) and 1-hydroxybenzotriazole (8.25 g, 53.9 mmol) in dichloromethane (250 ml) were added N-methoxymethanamine hydrochloride (5.26 g, 53.9 mmol) and triethylamine (19.83 g, 196 mmol). After stirring at RT overnight, the reaction mixture was diluted with water and washed with saturated sodium carbonate solution and brine. The organic layer was dried over anhydrous sodium sulfate, filtered and evaporated under reduced pressure to give 10.40 g (86% of theory) of the title compound.

¹H-NMR (400 MHz, CDCl₃): δ [ppm]=7.98 (s, 1H), 7.78 (d, 1H), 7.32 (d, 1H), 3.54 (s, 3H), 3.37 (s, 3H), 2.52 (s, 3H).

¹⁹F-NMR (376 MHz, CDCl₃): δ [ppm]=−61.86 (s, 3F).

### Intermediate 123A

### Diethyl 1-[2-(3,4-dimethylphenyl)-2-oxoethyl]-4-(trifluoromethyl)-1H-pyrazole-3,5-dicarboxylate

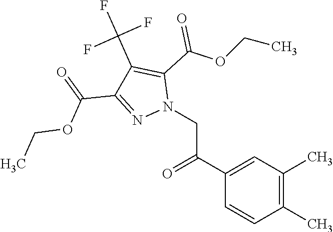 [SMILES: CCOC(=O)c1nn(CC(=O)c2ccc(C)c(C)c2)c(C(=O)OCC)c1C(F)(F)F]

To a solution of diethyl 4-(trifluoromethyl)-1H-pyrazole-3,5-dicarboxylate (Intermediate 32A, 1.00 g, 3.57 mmol) in acetone (31 ml) were added 2-bromo-1-(3,4-dimethylphenyl)ethanone (Intermediate 20A, 1.62 g, 60% purity, 4.28 mmol) and potassium carbonate (1.23 g, 8.92 mmol). The mixture was stirred at RT for 3 h. The reaction mixture was then filtered, the filter cake was washed with acetone, and the combined filtrates were evaporated to dryness to afford the crude title compound which was used in the next step without further purification. Yield: 2.20 g (quant. 83% purity according to LC/MS).

LC/MS [Method 3]: Rₜ=2.35 min; MS (ESIpos): m/z=427 [M+H]⁺.

### Intermediate 124A

### Ethyl 6-(3,4-dimethylphenyl)-4-oxo-3-(trifluoromethyl)-4,5-dihydropyrazolo[1,5-a]pyrazine-2-carboxylate

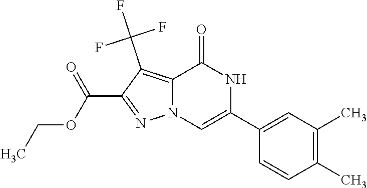 [SMILES: CCOC(=O)c1nn2cc(-c3ccc(C)c(C)c3)[nH]c(=O)c2c1C(F)(F)F]

A mixture of diethyl 1-[2-(3,4-dimethylphenyl)-2-oxoethyl]-4-(trifluoromethyl)-1H-pyrazole-3,5-dicarboxylate (Intermediate 123A, 2.20 g, 83% purity, 4.28 mmol) and ammonium acetate (13.2 g, 171 mmol) in acetic acid (86 ml) was heated to 110° C. for 4 days. After cooling to RT, the mixture was poured into water (400 ml), and the precipitate was collected by filtration, washed with ethyl acetate (10 ml) and MTBE (50 ml) and dried to afford the title compound. Yield: 1.02 g (60% of theory, 96% purity). The filtrate obtained above was concentrated to dryness, and the residue was triturated with MTBE. The solid was collected by filtration and dried to give a second crop of the title compound. Yield: 88.4 mg (5% of theory, 93% purity).

LC/MS [Method 3]: Rₜ=2.05 min; MS (ESIpos): m/z=380 [M+H]⁺.

¹H-NMR (500 MHz, DMSO-d₆): δ [ppm]=12.03 (s, 1H), 8.19 (s, 1H), 7.58 (s, 1H), 7.50 (d, 1H), 7.26 (d, 1H), 4.39 (q, 2H), 2.29 (s, 3H), 2.28 (s, 3H), 1.33 (t, 3H).

### Intermediate 125A

### 6-(3,4-Dimethylphenyl)-4-oxo-3-(trifluoromethyl)-4,5-dihydropyrazolo[1,5-a]pyrazine-2-carboxylic acid

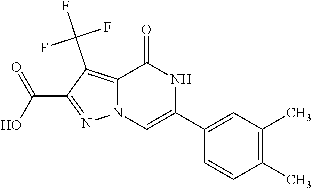 [SMILES: Cc1ccc(-c2cn3nc(C(=O)O)c(C(F)(F)F)c3c(=O)[nH]2)cc1C]

A mixture of ethyl 6-(3,4-dimethylphenyl)-4-oxo-3-(trifluoromethyl)-4,5-dihydropyrazolo[1,5-a]-pyrazine-2-carboxylate (Intermediate 124A, 1.10 g, 2.90 mmol) and lithium hydroxide (694 mg, 29.0 mmol) in ethanol (12 ml) and water (6.0 ml) was stirred at RT for 4 h. The ethanol was then removed under reduced pressure. The remaining aqueous phase was diluted with water to a final volume of 100 ml and brought to pH 2 by addition of 1.0 M hydrochloric acid. The precipitate to was collected by filtration and dried to afford the title compound. Yield 1.01 g (99% of theory).

LC/MS [Method 3]: Rₜ=1.42 min; MS (ESIpos): m/z=352 [M+H]⁺.

¹H-NMR (500 MHz, DMSO-d₆): δ [ppm]=11.99 (s, 1H), 8.14 (s, 1H), 7.58 (s, 1H), 7.49 (dd, 1H), 7.26 (d, 1H), 2.29 (s, 3H), 2.28 (s, 3H).

### Intermediate 126A

### Diethyl 1-[2-(3,4-dimethylphenyl)-2-oxoethyl]-4-(propan-2-yl)-1H-pyrazole-3,5-dicarboxylate

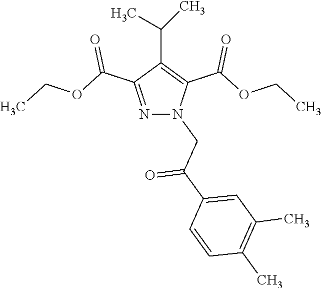 [SMILES: CCOC(=O)c1nn(CC(=O)c2ccc(C)c(C)c2)c(C(=O)OCC)c1C(C)C]

A mixture of diethyl 4-(propan-2-yl)-1H-pyrazole-3,5-dicarboxylate (Intermediate 16A, 2.00 g, 7.87 mmol), 2-bromo-1-(3,4-dimethylphenyl)ethan-1-one (Intermediate 20A, 2.55 g, 70% purity, 7.87 mmol) and potassium carbonate (2.72 g, 19.7 mmol) in acetone (69 ml) was stirred at RT overnight. Then, the mixture was filtered, and the filtrate was evaporated to afford the crude title compound which was used in the next step without further purification. Yield: 3.90 g (80% of theory, 65% purity).

LC/MS [Method 7]: Rₜ=1.26 min; MS (ESIpos): m/z=401 [M+H]⁺.

### Intermediate 127A

### Ethyl 6-(3,4-dimethylphenyl)-4-oxo-3-(propan-2-yl)-4,5-dihydropyrazolo[1,5-a]pyrazine-2-carboxylate

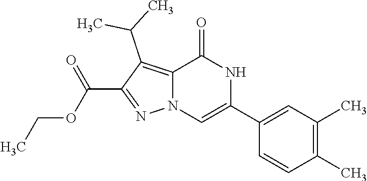 [SMILES: CCOC(=O)c1nn2cc(-c3ccc(C)c(C)c3)[nH]c(=O)c2c1C(C)C]

A mixture of diethyl 1-[2-(3,4-dimethylphenyl)-2-oxoethyl]-4-(propan-2-yl)-1H-pyrazole-3,5-dicarboxylate (Intermediate 126A, 3.90 g, 65% purity, 6.33 mmol) and ammonium acetate (19.5 g, 253 mmol) in acetic acid (63 ml) was heated to 110° C. for 48 h. After cooling to RT, the mixture was poured into water (700 ml). The precipitate was filtered off and dried under reduced pressure to afford the crude title compound which was used in the next step without further purification. Yield: 2.20 g (84% of theory, 85% purity).

LC/MS [Method 3]: Rₜ=2.22 min; MS (ESIpos): m/z=354 [M+H]⁺.

¹H-NMR (500 MHz, DMSO-d₆): δ [ppm]=11.47 (s, 1H), 8.03 (d, 1H), 7.56 (s, 1H), 7.47 (dd, 1H), 7.24 (d, 1H), 4.36-4.31 (m, 2H), 4.17-4.08 (m, 1H), 2.28 (s, 3H), 2.27 (s, 3H), 1.37 (d, 6H), 1.35-1.31 (m, 3H).

### Intermediate 128A

### 6-(3,4-Dimethylphenyl)-4-oxo-3-(propan-2-yl)-4,5-dihydropyrazolo[1,5-a]pyrazine-2-carboxylic acid

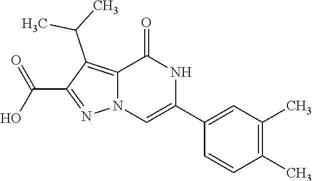 [SMILES: Cc1ccc(-c2cn3nc(C(=O)O)c(C(C)C)c3c(=O)[nH]2)cc1C]

A mixture of ethyl 6-(3,4-dimethylphenyl)-4-oxo-3-(propan-2-yl)-4,5-dihydropyrazolo[1,5-a]pyrazine-2-carboxylate (Intermediate 127A, 2.20 g, 6.22 mmol) and lithium hydroxide (745 mg, 31.1 mmol) in methanol (100 ml) and water (20 ml) was stirred at 50° C. for 2 h. After cooling to RT, the methanol was distilled off, and the aqueous residue was diluted with water to a final volume of 400 ml. Then, 1.0 M hydrochloric acid was added, and the precipitate was filtered off and dried under reduced pressure to afford the title compound. Yield: 2.00 g (96% of theory, 97% purity).

LC/MS [Method 7]: Rₜ=0.91 min; MS (ESIpos): m/z=326 [M+H]⁺.

¹H-NMR (500 MHz, DMSO-d₆): δ [ppm]=11.43 (s, 1H), 7.96 (s, 1H), 7.56 (s, 1H), 7.47 (dd, 1H), 7.24 (d, 1H), 4.18-4.08 (m, 1H), 2.28 (s, 3H), 2.27 (s, 3H), 1.37 (d, 6H).

### Intermediate 129A

### 2-Bromo-1-(2,3-dihydro-1H-inden-5-yl)ethan-1-one

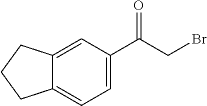 [SMILES: O=C(CBr)c1ccc2c(c1)CCC2]

Trimethylphenylammonium tribromide (11.7 g, 31.2 mmol) was added in portions to a solution of 1-(2,3-dihydro-1H-inden-5-yl)ethan-1-one (5.00 g, 31.2 mmol) in THF (50 ml) at RT. The mixture was subsequently stirred at RT. After completion of the reaction, the mixture was filtered, and the filtrate was concentrated to afford the crude product which was used in the next step without further purification. Yield: 10.0 g (94% of theory, 70% purity).

LC/MS [Method 7]: Rₜ=1.04 min; MS (ESIpos): m/z=239 [M+H]⁺.

### Intermediate 130A

### Diethyl 1-[2-(2,3-dihydro-1H-inden-5-yl)-2-oxoethyl]-1H-pyrazole-3,5-dicarboxylate

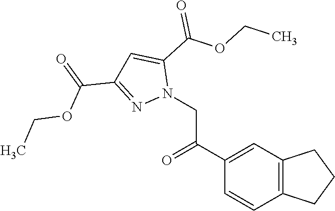 [SMILES: CCOC(=O)c1cc(C(=O)OCC)n(CC(=O)c2ccc3c(c2)CCC3)n1]

A mixture of diethyl 1H-pyrazole-3,5-dicarboxylate (5.95 g, 28.0 mmol), 2-bromo-1-(2,3-dihydro-1H-inden-5-yl)ethan-1-one (Intermediate 129A, 9.57 g, 70% purity, 28.0 mmol) and potassium carbonate (9.68 g, 70.1 mmol) in acetone (250 ml) was stirred at RT overnight. Then, the mixture was filtered, and the filtrate was concentrated to afford the crude product which was used in the next step without further purification. Yield: 10.4 g (65% of theory, 65% purity).

LC/MS [Method 7]: Rₜ=1.15 min; MS (ESIpos): m/z=371 [M+H]⁺.

### Intermediate 131A

### Ethyl 6-(2,3-dihydro-1H-inden-5-yl)-4-oxo-4,5-dihydropyrazolo[1,5-a]pyrazine-2-carboxylate

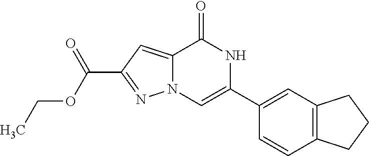 [SMILES: CCOC(=O)c1cc2c(=O)[nH]c(-c3ccc4c(c3)CCC4)cn2n1]

A mixture of diethyl 1-[2-(2,3-dihydro-1H-inden-5-yl)-2-oxoethyl]-1H-pyrazole-3,5-dicarboxylate (Intermediate 130A, 10.4 g, 65% purity, 18.3 mmol) and ammonium acetate (35.2 g, 456 mmol) in acetic acid (110 ml) was heated to 110° C. for 3 days. After cooling to RT, the mixture was poured in water (700 ml). The precipitate was filtered off, washed with MTBE (200 ml) and dried under reduced pressure to afford the crude title compound which was used in the next step without further purification. Yield: 3.30 g (51% of theory, 92% purity).

LC/MS [Method 3]: Rₜ=1.80 min; MS (ESIpos): m/z=324 [M+H]⁺.

¹H-NMR (500 MHz, DMSO-d₆): δ [ppm]=11.58 (br. s, 1H), 8.09 (s, 1H), 7.61 (s, 1H), 7.49 (dd, 1H), 7.40 (d, 1H), 7.34 (d, 1H), 4.35 (q, 2H), 2.94-2.88 (m, 4H), 2.09-2.02 (m, 2H), 1.33 (t, 3H).

### Intermediate 132A

### 6-(2,3-Dihydro-1H-inden-5-yl)-4-oxo-4,5-dihydropyrazolo[1,5-a]pyrazine-2-carboxylic acid

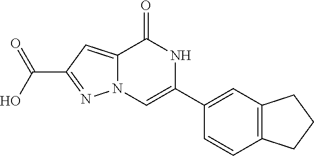 [SMILES: O=C(O)c1cc2c(=O)[nH]c(-c3ccc4c(c3)CCC4)cn2n1]

A mixture of ethyl 6-(2,3-dihydro-1H-inden-5-yl)-4-oxo-4,5-dihydropyrazolo[1,5-a]pyrazine-2-carboxylate (Intermediate 131A, 3.30 g, 10.2 mmol) and lithium hydroxide (1.22 g, 51.0 mmol) in methanol (100 ml) and water (20 ml) was stirred at 50° C. for 2 h. After cooling to RT, the methanol was distilled off, and the aqueous residue was diluted with water. Then, the mixture was acidified by addition of 1.0 M hydrochloric acid. The precipitate was filtered off and dried under reduced pressure to afford the title compound. Yield: 2.80 g (93% of theory).

LC/MS [Method 3]: Rₜ=1.33 min; MS (ESIneg): m/z=294 [M−H]⁻.

¹H-NMR (500 MHz, DMSO-d₆): δ [ppm]=13.57 (br. s, 1H), 11.69 (s, 1H), 8.05 (s, 1H), 7.61 (s, 1H), 7.50 (d, 1H), 7.36-7.33 (m, 2H), 2.96-2.87 (m, 4H), 2.10-2.01 (m, 2H).

### Intermediate 133A

### 2-Bromo-1-(5,6,7,8-tetrahydronaphthalen-2-yl)ethan-1-one

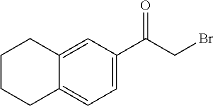 [SMILES: O=C(CBr)c1ccc2c(c1)CCCC2]

Trimethylphenylammonium tribromide (2.16 g, 5.74 mmol) was added in portions to a solution of 1-(5,6,7,8-tetrahydronaphthalen-2-yl)ethan-1-one (1.00 g, 5.74 mmol) in THF (10 ml) at RT. The mixture was subsequently stirred at RT. After completion of the reaction, the mixture was filtered, and the filtrate was concentrated to afford the crude product which was used in the next step without further purification. Yield: 1.70 g (78% of theory, 67% purity).

LC/MS [Method 7]: Rₜ=1.10 min; MS (ESIpos): m/z=255 [M+H]⁺.

### Intermediate 134A

### Diethyl 1-[2-oxo-2-(5,6,7,8-tetrahydronaphthalen-2-yl)ethyl]-1H-pyrazole-3,5-dicarboxylate

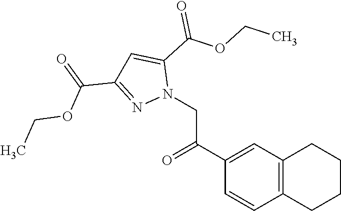 [SMILES: CCOC(=O)c1cc(C(=O)OCC)n(CC(=O)c2ccc3c(c2)CCCC3)n1]

A mixture of diethyl 1H-pyrazole-3,5-dicarboxylate (955 mg, 4.50 mmol), 2-bromo-1-(5,6,7,8-tetrahydronaphthalen-2-yl)ethan-1-one (Intermediate 133A, 1.70 g, 67% purity, 4.50 mmol) and potassium carbonate (1.55 g, 11.2 mmol) in acetone (40 ml) was stirred at RT overnight. The mixture was then filtered, and the filtrate was concentrated to afford the crude product which was used in the next step without further purification. Yield: 1.93 g (67% of theory, 60% purity).

LC/MS [Method 7]: Rₜ=1.21 min; MS (ESIpos): m/z=385 [M+H]⁺.

### Intermediate 135A

### Ethyl 4-oxo-6-(5,6,7,8-tetrahydronaphthalen-2-yl)-4,5-dihydropyrazolo[1,5-a]pyrazine-2-carboxylate

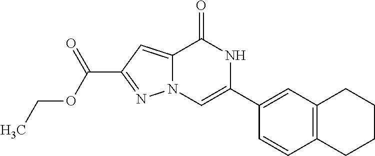 [SMILES: CCOC(=O)c1cc2c(=O)[nH]c(-c3ccc4c(c3)CCCC4)cn2n1]

A mixture of diethyl 1-[2-oxo-2-(5,6,7,8-tetrahydronaphthalen-2-yl)ethyl]-1H-pyrazole-3,5-dicarboxylate (Intermediate 134A, 1.93 g, 60% purity, 3.01 mmol) and ammonium acetate (5.80 g, 75.3 mmol) in acetic acid (30 ml) was heated to 110° C. for 3 days. After cooling to RT, the mixture was poured into water (200 ml). The precipitate was filtered off, washed with ethyl acetate (300 ml) and MTBE (200 ml) and dried under reduced pressure to afford the crude title compound which was used in the next step without further purification. Yield: 1.10 g (88% of theory, 81% purity).

LC/MS [Method 3]: Rₜ=1.93 min; MS (ESIpos): m/z=338 [M+H]⁺.

### Intermediate 136A

### 4-Oxo-6-(5,6,7,8-tetrahydronaphthalen-2-yl)-4,5-dihydropyrazolo[1,5-a]pyrazine-2-carboxylic acid

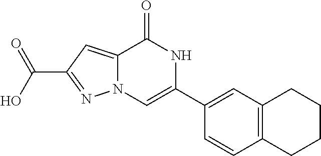 [SMILES: O=C(O)c1cc2c(=O)[nH]c(-c3ccc4c(c3)CCCC4)cn2n1]

A mixture of ethyl 4-oxo-6-(5,6,7,8-tetrahydronaphthalen-2-yl)-4,5-dihydropyrazolo[1,5-a]pyrazine-2-carboxylate (Intermediate 135A, 1.10 g, 3.26 mmol) and lithium hydroxide (390 mg, 16.3 mmol) in ethanol (60 ml) and water (10 ml) was stirred at 50° C. for 2 h. After cooling to RT, the ethanol was distilled off, and the aqueous residue was diluted with water. Then, the mixture was acidified by addition of 1.0 M hydrochloric acid. The precipitate was filtered off and dried under reduced pressure. The crude product was purified by preparative HPLC (Method P5) to afford the title compound. Yield: 340 mg (33% of theory, 98% purity).

LC/MS [Method 7]: Rₜ=0.80 min; MS (ESIpos): m/z=310 [M+H]⁺.

¹H-NMR (500 MHz, DMSO-d₆): δ [ppm]=13.05 (br. s, 1H), 11.67 (s, 1H), 8.07 (s, 1H), 7.50-7.44 (m, 2H), 7.35 (d, 1H), 7.17 (d, 1H), 2.81-2.73 (m, 4H), 1.80-1.73 (m, 4H).

### Intermediate 137A

### 4-(4-Fluorophenyl)-1-(2,2,2-trifluoroethyl)piperidin-4-ol

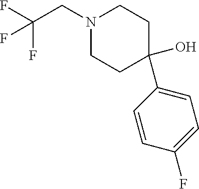 [SMILES: OC1(c2ccc(F)cc2)CCN(CC(F)(F)F)CC1]

To (4-fluorophenyl)magnesium bromide (2.8 ml, 1.0 M solution in THF, 2.8 mmol) was added a solution of 1-(2,2,2-trifluoroethyl)piperidin-4-one (426 mg, 2.35 mmol) in THF (8.0 ml) dropwise at 0° C. After complete addition, the cooling bath was removed and stirring was continued at RT for 3 h. The reaction was then quenched with ice-cold sodium bicarbonate solution, and the mixture was extracted with ethyl acetate (4×20 ml). The combined organic layers were dried over magnesium sulfate, filtered and evaporated to afford the crude title compound which was used in the next step without further purification. Yield: 585 mg (75% of theory, 84% purity).

LC/MS [Method 3]: Rₜ=1.49 min; MS (ESIpos): m/z=278 [M+H]⁺.

¹H-NMR (400 MHz, DMSO-d₆): δ [ppm]=7.55-7.48 (m, 2H), 7.11 (t, 2H), 4.91 (s, 1H), 3.18 (q, 2H), 2.86-2.67 (m, 4H), 1.97-1.85 (m, 2H), 1.60-1.49 (m, 2H).

### Intermediate 138A

### N-[4-(4-Fluorophenyl)-1-(2,2,2-trifluoroethyl)piperidin-4-yl]acetamide

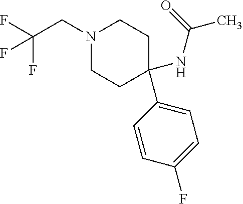 [SMILES: CC(=O)NC1(c2ccc(F)cc2)CCN(CC(F)(F)F)CC1]

Concentrated sulfuric acid (420 μl, 7.9 mmol) was added dropwise to a solution of 4-(4-fluoro-phenyl)-1-(2,2,2-trifluoroethyl)piperidin-4-ol (Intermediate 137A, 364 mg, 1.31 mmol) in acetonitrile (5.6 ml) at 0° C. The mixture was stirred at RT overnight. Then, ice-water was added, and the mixture was washed with MTBE (2×20 ml). The organic layers were discarded, and the aqueous layer was brought to basic pH by addition of 1.0 M aqueous sodium hydroxide solution. The aqueous layer was extracted with ethyl acetate (3×30 ml), and the combined organic layers were dried over magnesium sulfate, filtered and evaporated to afford the crude title compound which was used in the next step without further purification. Yield: 318 mg (73% of theory, 96% purity).

LC/MS [Method 7]: Rₜ=0.71 min; MS (ESIpos): m/z=319 [M+H]⁺.

### Intermediate 139A

### 4-(4-Fluorophenyl)-1-(2,2,2-trifluoroethyl)piperidin-4-amine

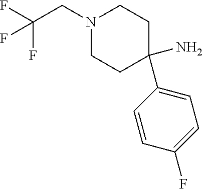 [SMILES: NC1(c2ccc(F)cc2)CCN(CC(F)(F)F)CC1]

A mixture of N-[4-(4-fluorophenyl)-1-(2,2,2-trifluoroethyl)piperidin-4-yl]acetamide (Intermediate 138A, 316 mg, 992 μmol) and hydrochloric acid (4.0 ml, 6.0 M) was heated to reflux for 4 days. After cooling to RT, ice-water (80 ml) was added, and the mixture was brought to basic pH by addition of 1.0 M aqueous sodium hydroxide solution. The aqueous phase was extracted with ethyl acetate (4×50 ml), and the combined organic layers were dried over magnesium sulfate, filtered and evaporated to afford the crude title compound which was used in the next step without further purification. Yield: 285 mg (67% of theory, 64% purity).

LC/MS [Method 4]: Rₜ=1.52 min; MS (ESIpos): m/z=277 [M+H]⁺.

### Intermediate 140A

### Diethyl 4-chloro-1-[2-(naphthalen-2-yl)-2-oxoethyl]-1H-pyrazole-3,5-dicarboxylate

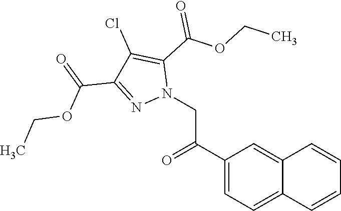 [SMILES: CCOC(=O)c1nn(CC(=O)c2ccc3ccccc3c2)c(C(=O)OCC)c1Cl]

To a solution of diethyl 4-chloro-1H-pyrazole-3,5-dicarboxylate (Intermediate 118A, 10.0 g, 88% purity, 35.7 mmol) in acetone (150 ml) were added 2-bromo-1-(2-naphthyl)ethanone (11.85 g, 75% purity, 35.7 mmol) and potassium carbonate (7.40 g, 53.5 mmol). The mixture was stirred at RT for 2 h. After filtering off the solids, the filtrate was concentrated under reduced pressure. The residue was suspensed in ethyl acetate/petroleum ether (300 ml, 1:10) and stirred for 30 minutes. The solid was collected by filtration to give the title compound. Yield: 11.00 g (67% of theory, 91% purity).

¹H-NMR (400 MHz, DMSO-d₆): δ [ppm]=8.81 (s, 1H), 8.15 (d, 1H), 8.08 (d, 1H), 8.04 (d, 1H), 8.02-7.97 (m, 1H), 7.79-7.69 (m, 1H), 7.69-7.64 (m, 1H), 6.39 (s, 2H), 4.33 (q, 2H), 4.20 (q, 2H), 1.30 (t, 3H), 1.10 (t, 3H).

### Intermediate 141A

### Ethyl 3-chloro-6-(naphthalen-2-yl)-4-oxo-4,5-dihydropyrazolo[1,5-a]pyrazine-2-carboxylate

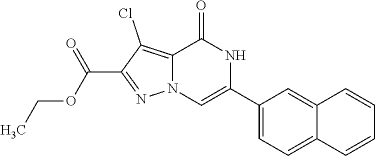 [SMILES: CCOC(=O)c1nn2cc(-c3ccc4ccccc4c3)[nH]c(=O)c2c1Cl]

Ammonium acetate (22.0 g, 285 mmol) was added to a solution of diethyl 4-chloro-1-[2-(naphthalen-2-yl)-2-oxoethyl]-1H-pyrazole-3,5-dicarboxylate (Intermediate 140A, 6.50 g, 91% purity, 14.3 mmol) in acetic acid (100 ml). The reaction mixture was stirred at 120° C. for 18 h. Then, the mixture was poured into ice-water, and the precipitate was collected by filtration, washed with water and dried in air to give the title compound. Yield: 5.20 g (94% of theory, 95% purity).

¹H-NMR (400 MHz, DMSO-d₆): δ [ppm]=11.92 (br. s, 1H), 8.36 (d, 1H), 8.32 (s, 1H), 8.02 (d, 1H), 7.99-7.93 (m, 2H), 7.85 (dd, 1H), 7.63-7.55 (m, 2H), 4.36 (q, 2H), 1.33 (t, 3H).

### Intermediate 142A

### 3-Chloro-6-(naphthalen-2-yl)-4-oxo-4,5-dihydropyrazolo[1,5-a]pyrazine-2-carboxylic acid

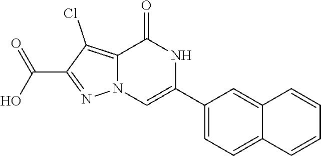 [SMILES: O=C(O)c1nn2cc(-c3ccc4ccccc4c3)[nH]c(=O)c2c1Cl]

An aqueous solution of sodium hydroxide (5.66 g, 141.4 mmol, dissolved in 40 ml water) was added to a mixture of ethyl 3-chloro-6-(2-naphthyl)-4-oxo-4,5-dihydropyrazolo[1,5-a]pyrazine-2-carboxylate (Intermediate 141A, 5.20 g, 14.1 mmol) and ethanol (100 ml). The mixture was stirred at RT for 2 h. Then, the ethanol was distilled off, and the remaining mixture was diluted with water (100 ml) and washed with MTBE (2×100 ml). The organic phase was discarded, and the aqueous layer was brought to pH 1 by addition of 2.0 M hydrochloric acid. The precipitate was collected by filtration, washed with water and dried in air to give the title compound. Yield: 4.42 g (90% of theory, 98% purity).

¹H-NMR (300 MHz, DMSO-d6): δ [ppm]=13.55 (br. s, 1H), 11.91 (s, 1H), 8.43-8.36 (m, 1H), 8.30 (s, 1H), 8.04 (d, 1H), 8.02-7.94 (m, 2H), 7.88 (dd, 1H), 7.72-7.51 (m, 2H).

### Intermediate 143A

### Quinoline-3-carbonyl chloride hydrochloride

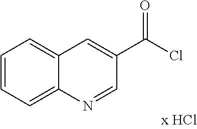 [SMILES: O=C(Cl)c1cnc2ccccc2c1]

A solution of quinoline-3-carboxylic acid (8.00 g, 46.2 mmol) in thionyl chloride (80 ml) was refluxed for 2 hours. After cooling to room temperature, the reaction solution was co-evaporated with toluene three times under reduced pressure to give the crude title compound which was directly used for the next step without further purification. Yield: 8.60 g (81% of theory).

¹H-NMR (300 MHz, DMSO-d₆): δ [ppm]=9.40 (s, 1H), 9.25-9.30 (m, 1H), 8.31-8.35 (m, 1H), 8.21-8.27 (m, 1H), 8.02-8.06 (m, 1H), 7.79-7.86 (m, 1H).

### Intermediate 144A

### 2-Bromo-1-(quinolin-3-yl)ethanone

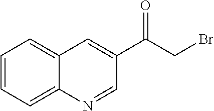 [SMILES: O=C(CBr)c1cnc2ccccc2c1]

To a solution of quinoline-3-carbonyl chloride hydrochloride (Intermediate 143A, 8.60 g, 37.7 mmol) in tetrahydrofuran (45 ml) and acetonitrile (45 ml) was added a solution of (trimethylsilyl)diazomethane in diethyl ether (37.7 ml, 2.0 M solution, 75.4 mmol) dropwise at room temperature. After stirring for 1 hour, 40% aqueous hydrobromic acid solution (17 ml) was added at 0° C. The resulting mixture was stirred for additional 30 minutes at room temperature, then adjusted to pH 8 with saturated aqueous sodium bicarbonate solution (100 ml) and extracted with ethyl acetate (2×200 ml). The combined organic layers were washed with water (2×200 ml) and brine (2×200 ml), dried over anhydrous sodium sulfate and filtered. The filtrate was evaporated to give the crude title compound which was directly used for the next step without further purification. Yield: 9.00 g (95% of theory).

### Intermediate 145A

### Diethyl 1-[2-oxo-2-(quinolin-3-yl)ethyl]-1H-pyrazole-3,5-dicarboxylate

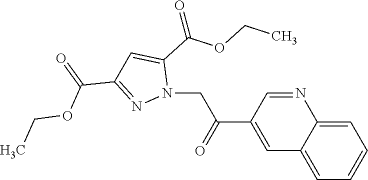 [SMILES: CCOC(=O)c1cc(C(=O)OCC)n(CC(=O)c2cnc3ccccc3c2)n1]

To a solution of 2-bromo-1-(quinolin-3-yl)ethanone (Intermediate 144A, 9.00 g, 36.0 mmol) in acetone (90 ml) were added diethyl 1H-pyrazole-3,5-dicarboxylate (7.60 g, 36.0 mmol) and potassium carbonate (9.93 g, 72.0 mmol). After stirring at room temperature overnight, the solids were filtered off. The filtrate was concentrated under reduced pressure, and the residue was purified by flash-chromatography on silica gel (eluent: petroleum ether/ethyl acetate 2:1) to give the title compound. Yield: 5.00 g (34% of theory, 93% purity).

LC/MS [Method 23]: Rₜ=0.91 min; MS (ESIpos): m/z=382 [M+H]⁺.

¹H-NMR (400 MHz, DMSO-d₆): δ [ppm]=9.38 (s, 1H), 9.23 (s, 1H), 8.22-8.24 (m, 1H), 8.14-8.17 (m, 1H), 7.97-8.01 (m, 1H), 7.77-7.81 (m, 1H), 7.40 (s, 1H), 6.43 (s, 2H), 4.33 (q, 2H), 4.21 (q, 2H), 1.30 (t, 3H), 1.19 (t, 3H).

### Intermediate 146A

### 3-[2-(Ethoxycarbonyl)-4-oxo-4,5-dihydropyrazolo[1,5-a]pyrazin-6-yl]quinolinium acetate

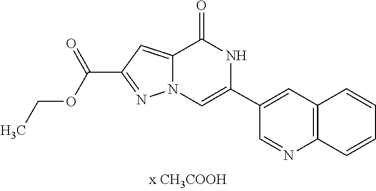 [SMILES: CCOC(=O)c1cc2c(=O)[nH]c(-c3cnc4ccccc4c3)cn2n1]

To a solution of diethyl 1-[2-oxo-2-(quinolin-3-yl)ethyl]-1H-pyrazole-3,5-dicarboxylate (Intermediate 145A, 5.00 g, 13.1 mmol) in acetic acid (60 ml) was added ammonium acetate (20.2 g, 262 mmol). The resulting mixture was stirred at 110° C. overnight. After cooling to room temperature, the reaction mixture was poured into ice-water. The precipitate was collected by filtration, washed with water (2×100 ml) and dried under reduced pressure to give the title compound. Yield: 2.60 g (48% of theory, 96% purity).

LC/MS [Method 6]: Rₜ=0.91 min; MS (ESIpos): m/z=335 [M+H]⁺.

¹H-NMR (400 MHz, DMSO-d₆): δ [ppm]=12.05 (s, 1H), 11.95 (s, 1H), 9.25 (s, 1H), 8.77 (s, 1H), 8.51 (s, 1H), 8.04-8.10 (m, 1H), 7.85 (t, 1H), 7.70-7.73 (m, 1H), 7.47 (s, 1H), 4.36 (q, 2H), 1.35 (t, 3H).

### Intermediate 147A

### 4-Oxo-6-(quinolin-3-yl)-4,5-dihydropyrazolo[1,5-a]pyrazine-2-carboxylic acid

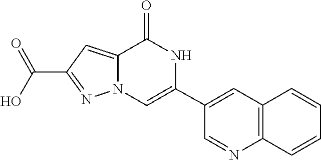 [SMILES: O=C(O)c1cc2c(=O)[nH]c(-c3cnc4ccccc4c3)cn2n1]

To a solution of 3-[2-(ethoxycarbonyl)-4-oxo-4,5-dihydropyrazolo[1,5-a]pyrazin-6-yl]quinolinium acetate (Intermediate 146A, 2.60 g, 6.6 mmol) in ethanol (54 ml) and water (6 ml) was added sodium hydroxide (2.64 g, 65.9 mmol). The mixture was stirred at room temperature for 2 h. Then, the reaction mixture was diluted with water (40 ml) and washed with ethyl acetate (2×60 ml). The organic phase was discarded, and the aqueous layer was adjusted to pH 7 with 1.0 M hydrochloric acid. The solid was collected by filtration and dried under reduced pressure to give the title compound. Yield: 1.91 g (91% of theory, 97% purity).

LC/MS [Method 2]: Rₜ=0.83 min; MS (ESIpos): m/z=307 [M+H]⁺.

¹H-NMR (300 MHz, DMSO-d₆): δ [ppm]=11.84 (br. s, 1H), 9.26 (s, 1H), 8.76 (s, 1H), 8.50 (s, 1H), 8.01-8.08 (m, 2H), 7.79-7.85 (m, 1H), 7.66-7.71 (m, 1H), 7.20 (s, 1H).

### Intermediate 148A

### 2-Bromo-1-(3,4-dimethylphenyl)propan-1-one

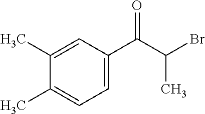

Trimethylphenylammonium tribromide (4.63 g, 12.3 mmol) was added to a solution of 1-(3,4-di-methylphenyl)propan-1-one (2.00 g, 12.3 mmol) in THF (22 ml). The resulting mixture was stirred at RT overnight. Then, the mixture was filtered, and the filtrate was evaporated to afford the crude title compound which was directly used in the next step without further purification. Yield: 4.56 g (quant., 65% purity).

LC/MS [Method 3]: Rₜ=2.09 min; MS (ESIpos): m/z=241 [M+H]⁺.

### Intermediate 149A

### Diethyl 1-[2-(3,4-dimethylphenyl)-1-methyl-2-oxoethyl]-4-(trifluoromethyl)pyrazole-3,5-dicarboxylate

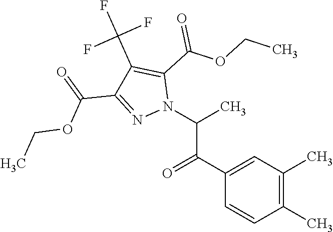 [SMILES: CCOC(=O)c1nn(C(C)C(=O)c2ccc(C)c(C)c2)c(C(=O)OCC)c1C(F)(F)F]

A mixture of diethyl 4-(trifluoromethyl)-1H-pyrazole-3,5-dicarboxylate (Intermediate 32A, 2.83 g, 10.1 mmol), 2-bromo-1-(3,4-dimethylphenyl)propan-1-one (Intermediate 148A, 4.50 g, 65% purity, 12.1 mmol) and potassium carbonate (3.49 g, 25.3 mmol) in acetone (89 ml) was stirred at RT for 3 h. The mixture was then filtered, and the filter cake was thoroughly washed with acetone. The combined filtrates were evaporated to leave the crude title compound which was directly used in the next step without further purification. Yield: 6.06 g (99% of theory, 73% purity).

LC/MS [Method 3]: Rₜ=2.38 min; MS (ESIpos): m/z=441 [M+H]⁺.

### Intermediate 150A

### Ethyl 6-(3,4-dimethylphenyl)-7-methyl-4-oxo-3-(trifluoromethyl)-4,5-dihydropyrazolo[1,5-a]pyrazine-2-carboxylate

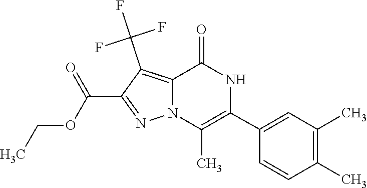 [SMILES: CCOC(=O)c1nn2c(C)c(-c3ccc(C)c(C)c3)[nH]c(=O)c2c1C(F)(F)F]

A mixture of diethyl 1-[2-(3,4-dimethylphenyl)-1-methyl-2-oxoethyl]-4-(trifluoromethyl)pyrazole-3,5-dicarboxylate (Intermediate 149A, 5.91 g, 83% purity, 11.1 mmol) and ammonium acetate (34.3 g, 446 mmol) in acetic acid (220 ml) was heated to 110° C. for 2 days. After cooling to RT, the mixture was poured into water (400 ml). The precipitate was collected by filtration and washed with ethyl acetate (10 ml) and MTBE (50 ml) to afford the title compound (1.90 g, 41% of theory, (95% purity). The mother liquor was evaporated, and the residue was triturated with MTBE. The solid was collected by filtration to give a second crop of the title compound (437 mg, 10% of theory).

LC/MS [Method 3]: Rₜ=2.17 min; MS (ESIpos): m/z=394 [M+H]⁺.

¹H-NMR (500 MHz, DMSO-d₆): δ [ppm]=11.90 (br. s, 1H), 7.30-7.27 (m, 2H), 7.25-7.21 (m, 1H), 4.41 (q, 2H), 2.32 (s, 3H), 2.30 (s, 3H), 2.29 (s, 3H), 1.33 (t, 3H).

### Intermediate 151A

### 6-(3,4-Dimethylphenyl)-7-methyl-4-oxo-3-(trifluoromethyl)-4,5-dihydropyrazolo[1,5-a]pyrazine-2-carboxylic acid

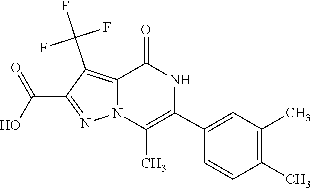 [SMILES: Cc1ccc(-c2[nH]c(=O)c3c(C(F)(F)F)c(C(=O)O)nn3c2C)cc1C]

A mixture of ethyl 6-(3,4-dimethylphenyl)-7-methyl-4-oxo-3-(trifluoromethyl)-4,5-dihydropyrazolo-[1,5-a]pyrazine-2-carboxylate (Intermediate 150A, 2.02 g, 5.14 mmol) and lithium hydroxide (1.23 g, 51.4 mmol) in ethanol (21 ml) and water (11 ml) was stirred at RT for 4 h. The ethanol was distilled off, and the aqueous layer was diluted with water to a final volume of 100 ml. Then, 1.0 M hydrochloric acid was added until pH 2 was reached, and the mixture was kept at 4° C. overnight. The precipitate was collected by filtration and dried under reduced pressure to afford the title compound. Yield: 1.58 g (79% of theory, 94% purity).

LC/MS [Method 3]: Rₜ=1.52 min; MS (ESIpos): m/z=366 [M+H]⁺.

¹H-NMR (500 MHz, DMSO-d₆): δ [ppm]=11.85 (s, 1H), 7.33-7.26 (m, 2H), 7.26-7.21 (m, 1H), 2.32 (s, 3H), 2.31-2.25 (m, 6H).

### Intermediate 152A

### Diethyl 1-[2-(3-chloro-4-methylphenyl)-2-oxoethyl]-1H-pyrazole-3,5-dicarboxylate

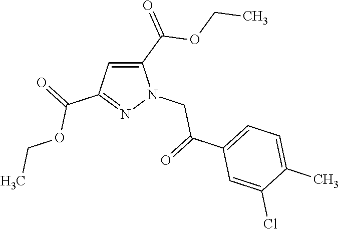 [SMILES: CCOC(=O)c1cc(C(=O)OCC)n(CC(=O)c2ccc(C)c(Cl)c2)n1]

A mixture of diethyl 1H-pyrazole-3,5-dicarboxylate (5.20 g, 24.5 mmol), 2-chloro-1-(3-chloro-4-methylphenyl)ethan-1-one (4.98 g, 24.5 mmol), potassium carbonate (3.73 g, 27.0 mmol) and water (3 drops) in acetone (100 ml) was stirred at RT overnight. The solids were then filtered off, and the filtrate was concentrated under reduced pressure. The residue was partitioned between dichloromethane and water. The layers were separated, and the organic layer was washed with water and brine, dried over sodium sulfate and filtered. The filtrate was concentrated under reduced pressure to afford the crude title compound which was directly used in the next step without further purification. Yield: 7.99 g (83% of theory, 97% purity).

LC/MS [Method 34]: Rₜ=1.39 min; MS (ESIpos): m/z=379/381 [M+H]⁺.

### Intermediate 153A

### Ethyl 6-(3-chloro-4-methylphenyl)-4-oxo-4,5-dihydropyrazolo[1,5-a]pyrazine-2-carboxylate

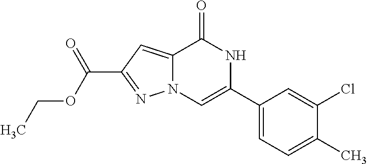 [SMILES: CCOC(=O)c1cc2c(=O)[nH]c(-c3ccc(C)c(Cl)c3)cn2n1]

A mixture of diethyl 1-[2-(3-chloro-4-methylphenyl)-2-oxoethyl]-1H-pyrazole-3,5-dicarboxylate (Intermediate 152A, 7.99 g, 97% purity, 20.5 mmol) and ammonium acetate (31.5 g, 409 mmol) in acetic acid (180 ml) was heated to reflux overnight. After cooling to RT, the mixture was poured into ice-water and neutralized by addition of aqueous sodium hydroxide solution. The precipitate was collected by filtration, washed with water and dichloromethane and dried to afford the title compound. Yield: 5.04 g (74% of theory, 99% purity).

LC/MS [Method 34]: Rₜ=1.15 min; MS (ESIpos): m/z=332/334 [M+H]⁺.

### Intermediate 154A

### 6-(3-Chloro-4-methylphenyl)-4-oxo-4,5-dihydropyrazolo[1,5-a]pyrazine-2-carboxylic acid

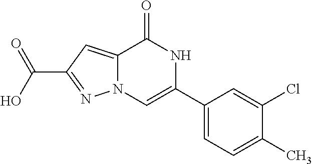 [SMILES: Cc1ccc(-c2cn3nc(C(=O)O)cc3c(=O)[nH]2)cc1Cl]

Aqueous sodium hydroxide solution (35 ml, 1.0 M, 35 mmol) was added to a suspension of ethyl 6-(3-chloro-4-methylphenyl)-4-oxo-4,5-dihydropyrazolo[1,5-a]pyrazine-2-carboxylate (Intermediate 153A, 1.45 g, 4.38 mmol) in ethanol (50 ml) and water (50 ml). The mixture was sonicated for 50 min and then stirred at RT overnight. The ethanol was distilled off, and the mixture was brought to pH 2 by addition of concentrated hydrochloric acid solution. The precipitate was collected by filtration, washed with water and dried under reduced pressure at 100° C. to afford the title compound. Yield: 1.32 g (99% of theory).

LC/MS [Method 34]: Rₜ=0.91 min; MS (ESIpos): m/z=304/306 [M+H]⁺.

### Intermediate 155A

### 3-(6-Methoxypyridin-3-yl)-1-methylazetidin-3-amine

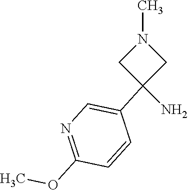

To a solution of tert.-butyl 3-azido-3-(6-methoxypyridin-3-yl)azetidine-1-carboxylate (Intermediate 10A, 5.4 g, 17.7 mmol) in THF (60 ml) was added lithium aluminium hydride (2.00 g, 53.1 mmol) in portions at 0° C. The reaction was stirred at room temperature for 30 min and then at 60° C. for 2 h. After this, the reaction was quenched with sodium sulfate decahydrate (15 g) at 0° C. The mixture was filtered, the filter cake was washed with methanol, and the combined filtrates were concentrated. The residue was purified by silica gel column chromatography [330 g, eluent: 0-10% methanol in dichloromethane (+0.5% triethylamine)] to give the title compound. Yield: 1.00 g (24% of theory, 81% purity).

LC/MS [Method 1]: Rₜ=0.47 min; MS (ESIpos): m/z=194 [M+H]⁺.

¹H-NMR (300 MHz, DMSO-d₆): δ [ppm]=8.31 (s, 1H), 7.90 (d, 1H), 6.78 (d, 1H), 3.83 (s, 3H), 3.20-3.30 (m, 2H), 3.11-3.17 (m, 2H), 2.30 (s, 3H).

### Intermediate 156A

### 1-(2-Naphthyl)propan-1-ol

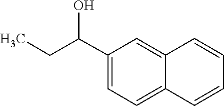

To a solution of 2-naphthaldehyde (10.0 g, 64.0 mmol) in THF (150 ml) was added ethyl-magnesium bromide (36.0 ml, 109 mmol, 3.0 M solution in diethyl ether) at −78° C. After stirring for 2 hours at room temperature, the reaction mixture was quenched with brine and extracted with ethyl acetate. The combined organic layers were dried over anhydrous sodium sulfate and filtered. The filtrate was concentrated under reduced pressure, and the residue was purified by flash-chromatography on silica gel (eluent: petroleum ether/ethyl acetate 3:1) to give the title compound. Yield: 10.50 g (86% of theory, 98% purity).

LC/MS [Method 5]: Rₜ=1.08 min; MS (ESIpos): m/z=169 [M+H−H₂O]⁺.

¹H-NMR (400 MHz, DMSO-d₆): δ [ppm]=7.80-7.89 (m, 4H), 7.44-7.51 (m, 3H), 5.26 (d, 1H), 4.60-4.64 (m, 1H), 1.67-1.74 (m, 2H), 0.85 (t, 3H).

### Intermediate 157A

### 1-(2-Naphthyl)propan-1-one

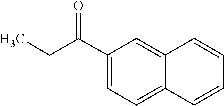

To a solution of 1-(2-naphthyl)propan-1-ol (Intermediate 156A, 10.5 g, 55.3 mmol) in dichloromethane (200 ml) was added Dess-Martin periodinane (46.9 g, 111 mmol). After stirring overnight at room temperature, the reaction mixture was concentrated under reduced pressure. The residue was purified by flash-chromatography on silica gel (eluent:petroleum ether/ethyl acetate 7:3) to give the title compound. Yield: 10.30 g (98% of theory, 97% purity).

LC/MS [Method 20]: Rₜ=1.08 min; MS (ESIpos): m/z=185 [M+H]⁺.

¹H-NMR (300 MHz, DMSO-d₆): δ [ppm]=8.68 (s, 1H), 8.13 (d, 1H), 7.97-8.04 (m, 3H), 7.59-7.70 (m, 2H), 3.20 (q, 2H), 1.15 (t, 3H).

### Intermediate 158A

### 2-Bromo-1-(2-naphthyl)propan-1-one

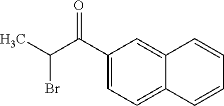

To a solution of 1-(2-naphthyl)propan-1-one (Intermediate 157A, 10.3 g, 54.6 mmol) in chloroform (200 ml) was added phenyltrimethylammonium tribromide (20.5 g, 54.6 mmol). After stirring overnight at room temperature, the reaction mixture was concentrated under reduced pressure. The residue was purified by flash-chromatography on silica gel (eluent: petroleum ether/ethyl acetate 4:1) to give the title compound. Yield: 12.50 g (82% of theory, 94% purity).

LC/MS [Method 20]: Rₜ=1.15 min; MS (ESIpos): m/z=263/265 [M+H]⁺.

¹H-NMR (300 MHz, DMSO-d₆): δ [ppm]=8.79 (s, 1H), 7.97-8.20 (m, 4H), 7.62-7.76 (m, 2H), 6.01 (q, 1H), 1.85 (d, 3H).

### Intermediate 159A

### Diethyl 1-[1-(2-naphthyl)-1-oxopropan-2-yl]-1H-pyrazole-3,5-dicarboxylate

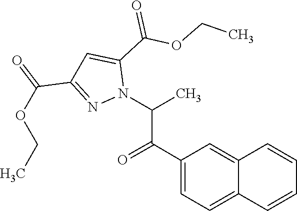 [SMILES: CCOC(=O)c1cc(C(=O)OCC)n(C(C)C(=O)c2ccc3ccccc3c2)n1]

To a solution of 2-bromo-1-(2-naphthyl)propan-1-one (Intermediate 158A, 6.48 g, 23.3 mmol, 94% purity) in acetone (100 ml) were added diethyl 1H-pyrazole-3,5-dicarboxylate (4.50 g, 21.2 mmol) and potassium carbonate (7.33 g, 53.0 mmol). After stirring at room temperature overnight, the solids were filtered off. The filtrate was concentrated under reduced pressure, and the residue was purified by flash-chromatography on silica gel (eluent: petroleum ether/ethyl acetate 3:1) to give the title compound. Yield: 8.10 g (94% of theory, 97% purity).

LC/MS [Method 20]: Rₜ=1.30 min; MS (ESIpos): m/z=395 [M+H]⁺.

¹H-NMR (300 MHz, DMSO-d₆): δ [ppm]=8.76 (s, 1H), 7.97-8.16 (m, 4H), 7.66-7.72 (m, 2H), 7.37 (s, 1H), 7.10 (q, 1H), 4.32 (q, 2H), 4.20 (q, 2H), 1.85 (d, 3H), 1.31 (t, 3H), 1.18 (t, 3H).

### Intermediate 160A

### Ethyl 7-methyl-6-(2-naphthyl)-4-oxo-4,5-dihydropyrazolo[1,5-a]pyrazine-2-carboxylate

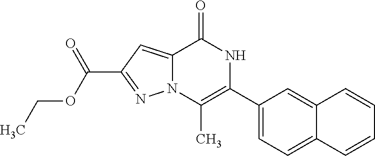 [SMILES: CCOC(=O)c1cc2c(=O)[nH]c(-c3ccc4ccccc4c3)c(C)n2n1]

To a solution of diethyl 1-[1-(2-naphthyl)-1-oxopropan-2-yl]-1H-pyrazole-3,5-dicarboxylate (Intermediate 159A, 8.10 g, 20.0 mmol) in acetic acid (135 ml) was added ammonium acetate (30.8 g, 400 mmol) at room temperature. The resulting mixture was stirred at 110° C. for 18 h. After cooling to room temperature, the reaction mixture was diluted with water. The precipitate was collected by filtration, washed with water and dried under reduced pressure to give 6.10 g of a mixture of the title compound and the ester hydrolysis by-product (see Intermediate 161A) which was directly used for the next step without further purification (total yield 82% of theory).

LC/MS [Method 10]: Rₜ=1.07 min; MS (ESIpos): m/z=348 [M+H]⁺.

¹H-NMR (300 MHz, DMSO-d₆): δ [ppm]=11.75 (br. s, 1H), 8.01-8.12 (m, 4H), 7.43-7.66 (m, 4H), 4.38 (q, 2H), 2.40 (s, 3H), 1.35 (t, 3H).

### Intermediate 161A

### 7-Methyl-6-(2-naphthyl)-4-oxo-4,5-dihydropyrazolo[1,5-a]pyrazine-2-carboxylic acid

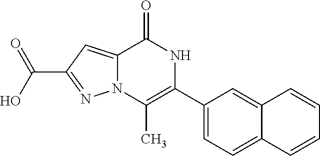 [SMILES: Cc1c(-c2ccc3ccccc3c2)[nH]c(=O)c2cc(C(=O)O)nn12]

To a solution of crude ethyl 7-methyl-6-(2-naphthyl)-4-oxo-4,5-dihydropyrazolo[1,5-a]pyrazine-2-carboxylate (Intermediate 160A, 6.10 g) in ethanol (100 ml) were added sodium hydroxide (5.38 g, 135 mmol) and water (50 ml). After stirring at room temperature for 2 h, the reaction mixture was diluted with water and washed with ethyl acetate. The organic phases were discarded, and the aqueous layer was adjusted to pH 3 with 3.0 M hydrochloric acid solution. The precipitate was collected by filtration, washed with water and dried under reduced pressure to give the title compound. Yield: 3.04 g (68% of theory, 96% purity).

LC/MS [Method 32]: Rₜ=1.19 min; MS (ESIpos): m/z=320 [M+H]⁺.

¹H-NMR (400 MHz, DMSO-d₆): δ [ppm]=11.68 (br. s, 1H), 8.12 (s, 1H), 8.00-8.06 (m, 3H), 7.59-7.65 (m, 3H), 7.40 (s, 1H), 2.40 (s, 3H).

### Intermediate 162A

### N-Methoxy-N-methylquinoline-2-carboxamide

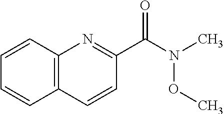 [SMILES: CON(C)C(=O)c1ccc2ccccc2n1]

To a suspension of quinoline-2-carboxylic acid (10.00 g, 57.75 mmol), 1-(3-dimethylaminopropyl)-3-ethylcarbodiimide hydrochloride (12.18 g, 63.52 mmol) and 1-hydroxybenzotriazole (9.73 g, 63.52 mmol) in dichloromethane (150 ml) were added N-methoxymethanamine hydrochloride (6.20 g, 63.52 mmol) and triethylamine (23.37 g, 230.98 mmol), and the mixture was stirred at room temperature for 2 h. The reaction mixture was then diluted with water (100 ml) and washed with aqueous sodium carbonate solution (3×100 ml) and water (3×100 ml). The organic layer was dried over sodium sulfate, filtered and concentrated to give the title compound. Yield: 8.20 g (63% of theory, 96% purity).

LC/MS [Method 10]: Rₜ=0.81 min; MS (ESIpos): m/z=217 [M+H]⁺.

¹H-NMR (400 MHz, CDCl₃): δ [ppm]=8.25 (d, 1H), 8.14 (d, 1H), 7.86 (d, 1H), 7.65-7.78 (m, 2H), 7.60 (t, 1H), 3.76 (s, 3H), 3.44 (s, 3H).

### Intermediate 163A

### 1-(Quinolin-2-yl)ethan-1-one

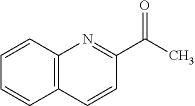

A solution of N-methoxy-N-methylquinoline-2-carboxamide (Intermediate 162A, 8.00 g, 35.71 mmol, 96% purity) in THF (100 ml) was degassed with nitrogen three times. Subsequently, methylmagnesium bromide (39.3 ml, 1.0 M solution in THF) was added slowly at 0° C. The mixture was stirred at room temperature for 2 h. Then, water (50 ml) was added, and the mixture was extracted with ethyl acetate (5×100 ml). The combined organic layers were dried over anhydrous sodium sulfate, filtered and concentrated to give the title compound. Yield: 6.07 g (96% of theory, 97% purity).

LC/MS [Method 6]: Rₜ=1.04 min; MS (ESIpos): m/z=172 [M+H]⁺.

¹H-NMR (400 MHz, CDCl₃): δ [ppm]=8.26 (d, 1H), 8.20 (d, 1H), 8.13 (d, 1H), 7.87 (d, 1H), 7.76-7.81 (m, 1H), 7.63-7.67 (m, 1H), 2.87 (s, 3H).

### Intermediate 164A

### 2-Bromo-1-(quinolin-2-yl)ethan-1-one

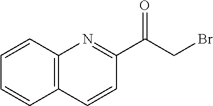 [SMILES: O=C(CBr)c1ccc2ccccc2n1]

Bromine (5.01 g, 31.35 mmol) was added dropwise to a solution of 1-(quinolin-2-yl)ethanone (Intermediate 163A, 5.50 g, 31.3 mmol, 97% purity) in 40% aqueous hydrobromic acid (15 ml) at 60° C., and the mixture was kept at 60° C. for further 2 h. Then, aqueous sodium carbonate solution was added at 0° C. to adjust the pH to 9. After this, the mixture was extracted with ethyl acetate (3×50 ml). The combined organic phases were dried and concentrated to give the title compound. Yield: 6.20 g (67% of theory, 86% purity).

LC/MS [Method 6]: Rₜ=1.17 min; MS (ESIpos): m/z=250 [M+H]⁺.

¹H-NMR (400 MHz, CDCl₃): δ [ppm]=8.31 (d, 1H), 8.15-8.20 (m, 2H), 7.90 (d, 1H), 7.79-7.83 (m, 1H), 7.66-7.70 (m, 1H), 5.08 (s, 2H).

### Intermediate 165A

### Diethyl 1-[2-oxo-2-(quinolin-2-yl)ethyl]-1H-pyrazole-3,5-dicarboxylate

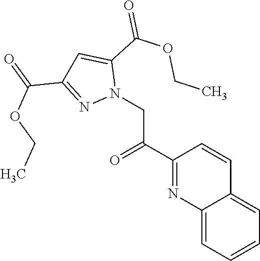 [SMILES: CCOC(=O)c1cc(C(=O)OCC)n(CC(=O)c2ccc3ccccc3n2)n1]

A mixture of diethyl 1H-pyrazole-3,5-dicarboxylate (3.00 g, 14.14 mmol), 2-bromo-1-(quinolin-2-yl)ethanone (Intermediate 164A, 3.54 g, 14.14 mmol) and potassium carbonate (2.15 g, 15.55 mmol) in acetone (40 ml) was stirred at room temperature overnight. After filtering off the solids, the filtrate was evaporated and the residue partitioned between dichloromethane and water. The organic phase was washed with water and brine, dried over sodium sulfate and concentrated. The crude product was purified by flash-chromatography on silica gel (80 g, eluent: 0-20% ethyl acetate in petroleum ether) to give the title compound. Yield: 4.10 g (76% of theory, 76% purity).

LC/MS [Method 1]: Rₜ=2.10 min; MS (ESIpos): m/z=382 [M+H]⁺.

¹H-NMR (300 MHz, DMSO-d₆): δ [ppm]=8.66 (d, 1H), 8.08-8.24 (m, 3H), 7.72-7.99 (m, 2H), 7.39 (s, 1H), 6.49 (s, 2H), 4.33 (q, 2H), 4.18 (q, 2H), 1.32 (t, 3H), 1.15 (t, 3H).

### Intermediate 166A

### Ethyl 4-oxo-6-(quinolin-2-yl)-4,5-dihydropyrazolo[1,5-a]pyrazine-2-carboxylate

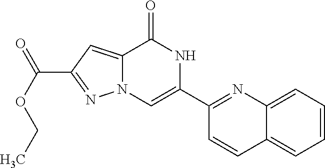 [SMILES: CCOC(=O)c1cc2c(=O)[nH]c(-c3ccc4ccccc4n3)cn2n1]

Ammonium acetate (16.57 g, 215 mmol) was added to a solution of diethyl 1-[2-oxo-2-(quinolin-2-yl)ethyl]-1H-pyrazole-3,5-dicarboxylate (Intermediate 165A, 4.10 g, 10.75 mmol) in acetic acid (50 ml), and the mixture was stirred at 110° C. overnight. After cooling to RT, the solution was poured into ice-water. The precipitate was collected by filtration, washed with water and dried in air to give the title compound. Yield: 3.30 g (92% of theory, 71% purity).

LC/MS [Method 10]: Rₜ=1.05 min; MS (ESIpos): m/z=335 [M+H]⁺.

¹H-NMR (300 MHz, DMSO-d₆): δ [ppm]=9.00 (s, 1H), 8.51 (d, 1H), 8.27 (d, 1H), 8.15 (d, 1H), 8.03 (d, 1H), 7.48-7.89 (m, 3H), 7.45 (s, 1H), 4.33 (q, 2H), 1.31 (t, 3H).

### Intermediate 167A

### 4-Oxo-6-(quinolin-2-yl)-4,5-dihydropyrazolo[1,5-a]pyrazine-2-carboxylic acid

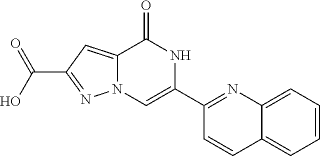 [SMILES: O=C(O)c1cc2c(=O)[nH]c(-c3ccc4ccccc4n3)cn2n1]

A 100 ml round-bottom flask was charged with ethyl 4-oxo-6-(quinolin-2-yl)-4,5-dihydropyrazolo-[1,5-a]pyrazine-2-carboxylate (Intermediate 166A, 3.30 g, 9.87 mmol) and ethanol (20 ml). An aqueous solution of sodium hydroxide (15 ml, 3.0 M) was added, and the mixture was stirred at room temperature for 4 h. Then, the pH value of the mixture was adjusted to 6 with 3.0 M hydrochloric acid. The precipitate was collected by filtration, washed with water and dried to afford the title compound. Yield: 931.7 mg (27% of theory, 88% purity).

LC/MS [Method 2]: Rₜ=2.56 min; MS (ESIpos): m/z=307 [M+H]⁺.

¹H-NMR (300 MHz, DMSO-d₆): δ [ppm]=13.39 (br. s, 1H), 11.12 (s, 1H), 9.03 (s, 1H), 8.55 (d, 1H), 8.31 (d, 1H), 8.19 (d, 1H), 8.05 (d, 1H), 7.85-7.88 (m, 1H), 7.66-7.71 (m, 1H), 7.45 (s, 1H).

### Intermediate 168A

### Dimethyl 1-[2-(4-chloro-3-methylphenyl)-2-oxoethyl]-1H-pyrazole-3,5-dicarboxylate

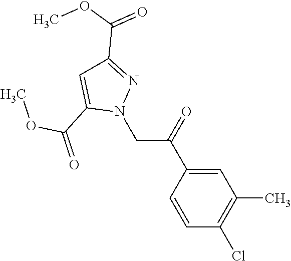

Dimethyl 1H-pyrazole-3,5-dicarboxylate (310 mg, 1.68 mmol), 2-bromo-1-(4-chloro-3-methyl-phenyl)ethan-1-one (500 mg, 2.02 mmol) and potassium carbonate (256 mg, 1.85 mmol) in acetone (6.5 ml, containing 2 drops of water) were stirred at room temperature for 1.5 h. After filtering off the solids, the filtrate was partitioned between dichloromethane and water. The aqueous phase was extracted three times with dichloromethane, and the combined organic phases were dried over sodium sulfate, filtered and concentrated to provide the title product. Yield: 706 mg (99% of theory, 83% purity).

LC/MS [Method 7]: Rₜ=1.04 min; MS (ESIpos): m/z=351 [M+H]⁺.

### Intermediate 169A

### Methyl 6-(4-chloro-3-methylphenyl)-4-oxo-4,5-dihydropyrazolo[1,5-a]pyrazine-2-carboxylate

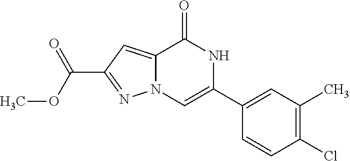

A solution of dimethyl 1-[2-(4-chloro-3-methylphenyl)-2-oxoethyl]-1H-pyrazole-3,5-dicarboxylate (Intermediate 168A, 706 mg, 83% purity, 1.67 mmol) and ammonium acetate (2.58 g, 33.4 mmol) in acetic acid (17 ml) was heated to reflux (bath temperature 138° C.) overnight. After cooling to RT, the reaction mixture was diluted with ice/water and neutralized with sodium hydroxide. The precipitate was filtered off, washed with water and dichloromethane and dried under high vacuum to provide the title compound. Yield: 417 mg (79% of theory).

LC/MS [Method 3]: Rₜ=1.64 min; MS (ESIpos): m/z=318 [M+H]⁺.

### Intermediate 170A

### 6-(4-Chloro-3-methylphenyl)-4-oxo-4,5-dihydropyrazolo[1,5-a]pyrazine-2-carboxylic acid

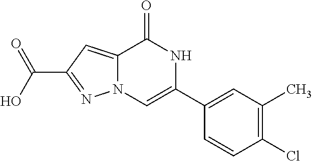 [SMILES: Cc1cc(-c2cn3nc(C(=O)O)cc3c(=O)[nH]2)ccc1Cl]

A solution of methyl 6-(4-chloro-3-methylphenyl)-4-oxo-4,5-dihydropyrazolo[1,5-a]pyrazine-2-carboxylate (Intermediate 169A, 417 mg, 1.31 mmol) in THF (11 ml) and water (3.5 ml) was treated with lithium hydroxide (157 mg, 6.57 mmol) and stirred at RT for 3 h. The THF was then evaporated under reduced pressure. To the remaining mixture, water was added until complete dissolution, and the mixture was acidified with 1.0 M hydrochloric acid. The precipitate was collected by filtration, washed with water and dried under high vacuum to provide the title compound. Yield: 395 mg (99% of theory).

LC/MS [Method 3]: Rₜ=1.30 min; MS (ESIpos): m/z=304 [M+H]⁺.

### Intermediate 171A

### 5-Fluoro-2,3-dihydro-1,4-benzodioxine

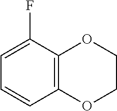 [SMILES: Fc1cccc2c1OCCO2]

A suspension of 3-fluorobenzene-1,2-diol (7.50 g, 58.5 mmol), 1,2-dibromoethane (5.6 ml, 64 mmol) and cesium carbonate (57.2 g, 176 mmol) in DMF (70 ml) was stirred at 120° C. for 18 h. After cooling down to RT, the mixture was partitioned between water and ethyl acetate. After phase separation, the aqueous layer was extracted three times with ethyl acetate. The combined organic extracts were washed with water and brine, dried over sodium sulfate, filtered and evaporated. The crude product was purified by silica gel chromatography (eluent: 5-40% ethyl acetate in cyclohexane) to provide the title compound. Yield: 3.81 g (42% of theory).

¹H-NMR (400 MHz, DMSO-d₆): δ [ppm]=6.82-6.74 (m, 2H), 6.74-6.67 (m, 1H), 4.31-4.27 (m, 6H).

### Intermediate 172A

### 6-Bromo-5-fluoro-2,3-dihydro-1,4-benzodioxine

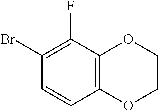 [SMILES: Fc1c(Br)ccc2c1OCCO2]

5-Fluoro-2,3-dihydro-1,4-benzodioxine (Intermediate 171A, 3.81 g, 24.7 mmol) was dissolved in chloroform (73 ml), cooled to 0° C. and treated with sodium acetate (2.33 g, 28.5 mmol). Subsequently, bromine (1.3 ml, 26 mmol) was added dropwise. When the addition was complete, the cooling bath was removed, and the reaction mixture was allowed to warm up to RT and stirred for 4 h. The reaction mixture was then quenched with 40 ml water and 10 ml saturated aqueous sodium sulfite solution. Extractive work-up with dichloromethane was performed. The organic extracts were combined and washed with brine, dried, filtered and evaporated. The crude product was purified by silica gel chromatography eluting with cyclohexane/5-40% ethyl acetate to provide the title compound. Yield: 4.42 g (53% of theory, 69% purity).

GC/MS [Method 35]: Rₜ=5.11 min; MS (EIpos): m/z=232 [M]⁺.

### Intermediate 173A

### 1-(5-Fluoro-2,3-dihydro-1,4-benzodioxin-6-yl)ethan-1-one

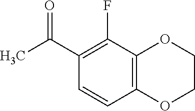

A solution of 6-bromo-5-fluoro-2,3-dihydro-1,4-benzodioxine (Intermediate 172A, 490 mg, 2.10 mmol), palladium(II) acetate (11.8 mg, 52.6 μmol) and 1,3-bis(diphenylphosphino)propane (dppp) (43.4 mg, 105 μmol) in 1-methyl-3-butylimidazolium-tetrafluoroborate (4.9 ml) was treated with 1-(ethenyloxy)butane (1.4 ml, 11 mmol) and triethylamine (350 μl, 2.5 mmol). The mixture was stirred at 115° C. for 24 h. Then, more palladium(II) acetate (23.6 mg) and 1,3-bis(diphenylphosphino)propane (87.3 mg) were added, and the reaction mixture was stirred at 115° C. for further three days. The reaction mixture was then cooled down to RT, 10 ml of 5% hydrochloric acid were added, and the mixture was stirred for 30 min. The aqueous phase was extracted with 30 ml dichloromethane. The organic phase was washed with water, dried and evaporated. The crude product was purified by chromatography on silica gel eluting with cyclohexane/0-30% ethyl acetate to provide the title compound. Yield: 104 mg (25% of theory).

¹H-NMR (400 MHz, DMSO-d₆): δ [ppm]=7.31 (t, 1H), 6.82 (dd, 1H), 4.42-4.29 (m, 4H), 2.52-2.50 (m, 3H).

### Intermediate 174A

### 2-Bromo-1-(5-fluoro-2,3-dihydro-1,4-benzodioxin-6-yl)ethan-1-one

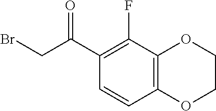 [SMILES: O=C(CBr)c1ccc2c(c1F)OCCO2]

A solution of 1-(5-fluoro-2,3-dihydro-1,4-benzodioxin-6-yl)ethan-1-one (Intermediate 173A, 1.27 g, 6.47 mmol) in acetic acid (10 ml) was warmed to 40° C. and treated dropwise under stirring with bromine (330 μl, 6.5 mmol). When the addition was complete, the reaction mixture was cooled in a cold water bath and then concentrated to dryness under reduced pressure. The material thus obtained was used in the next step without further purification. Yield: 1.70 g (57% of theory, 60% purity).

GC/MS [Method 35]: Rₜ=6.86 min; MS (EIpos): m/z=274 [M]⁺.

### Intermediate 175A

### Dimethyl 1-[2-(5-fluoro-2,3-dihydro-1 ,4-benzodioxin-6-yl)-2-oxoethyl]-1H-pyrazole-3,5-dicarboxylate

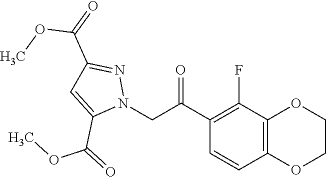 [SMILES: COC(=O)c1cc(C(=O)OC)n(CC(=O)c2ccc3c(c2F)OCCO3)n1]

Dimethyl 1H-pyrazole-3,5-dicarboxylate (469 mg, 2.54 mmol), 2-bromo-1-(5-fluoro-2,3-dihydro-1,4-benzodioxin-6-yl)ethan-1-one (Intermediate 174A, 700 mg, 2.54 mmol) and potassium carbonate (387 mg, 2.80 mmol) in acetone (9.8 ml, containing 2 drops of water) were stirred at room temperature for 18 h. Water (40 ml) was then added, and the mixture was stirred for 30 min before filtering. The solid was washed with water and pentane and dried under high vacuum to afford the title compound. Yield: 750 mg (76% of theory, 98% purity).

LC/MS [Method 7]: Rₜ=0.90 min; MS (ESIpos): m/z=379 [M+H]⁺.

### Intermediate 176A

### Methyl 6-(5-fluoro-2,3-dihydro-1,4-benzodioxin-6-yl)-4-oxo-4,5-dihydropyrazolo[1,5-a]pyrazine-2-carboxylate

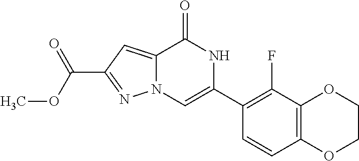 [SMILES: COC(=O)c1cc2c(=O)[nH]c(-c3ccc4c(c3F)OCCO4)cn2n1]

A solution of dimethyl 1-[2-(5-fluoro-2,3-dihydro-1,4-benzodioxin-6-yl)-2-oxoethyl]-1H-pyrazole-3,5-dicarboxylate (Intermediate 175A, 750 mg, 1.98 mmol) and ammonium acetate (3.82 g, 49.6 mmol) in acetic acid (8.0 ml) was heated to reflux (bath temperature 125° C.). After 6 h, the reaction mixture was evaporated under reduced pressure at 40° C. bath temperature. The residue was treated with water (2 ml) and dichloromethane (5 ml), and the pH of the mixture was carefully adjusted to 6 using concentrated aqueous sodium hydroxide solution. Then, extractive work-up was performed with dichloromethane. The combined organic layers were evaporated to provide the title compound. Yield: 548 mg (73% of theory, 91% purity).

LC/MS [Method 3]: Rₜ=1.34 min; MS (ESIpos): m/z=346 [M+H]⁺.

### Intermediate 177A

### 6-(5-Fluoro-2,3-dihydro-1,4-benzodioxin-6-yl)-4-oxo-4,5-dihydropyrazolo[1,5-a]pyrazine-2-carboxylic acid

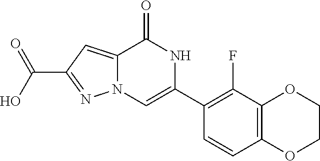 [SMILES: O=C(O)c1cc2c(=O)[nH]c(-c3ccc4c(c3F)OCCO4)cn2n1]

A solution of methyl 6-(5-fluoro-2,3-dihydro-1,4-benzodioxin-6-yl)-4-oxo-4,5-dihydropyrazolo[1,5-a]-pyrazine-2-carboxylate (Intermediate 176A, 495 mg, 91% purity, 1.30 mmol) in THF (9.0 ml) and water (2.7 ml) was treated with lithium hydroxide (125 mg, 5.22 mmol). The mixture was stirred at 40° C. for 3 days. Then, the THF was evaporated under reduced pressure. To the remaining mixture, water was added until complete dissolution, and the mixture was acidified with 1.0 M hydrochloric acid. The precipitate was collected by filtration, washed with water and dried under high vacuum to provide the title compound. Yield: 443 mg (99% of theory, 97% purity).

LC/MS [Method 3]: Rₜ=0.96 min; MS (ESIpos): m/z=332 [M+H]⁺.

### Intermediate 178A

### (1R)-1-(6-Methoxypyridin-3-yl)ethan-1-amine dihydrochloride

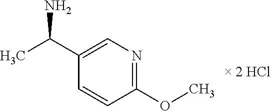

A solution of (S)—N-[(1R)-1-(6-methompyridin-3-yl)ethyl]-2-methylpropane-2-sulfinamide (Intermediate 48A, 450 mg, 1.76 mmol) in methanol (4.1 ml) was treated with hydrogen chloride (4.0 M solution in 1,4-dioxane, 4.4 ml, 18 mmol) under cooling. The reaction mixture was stirred at RT for 3 h and then evaporated under reduced pressure. Diethyl ether (10 ml) and dioxane (2 ml) were added, and the mixture was further stirred for 1 h and then evaporated again. The residue was dissolved in dichloromethane (4.0 ml) and saturated aqueous sodium bicarbonate solution (1.0 ml). The resulting solution was directly charged to a silica gel column for chromatographic separation (eluent: dichloromethane/0-17% methanol containing 10% concentrated aqueous ammonia). The material thus obtained was dissolved in 7 ml of water/acetonitrile (1:1), 0.9 ml of hydrogen chloride solution (4.0 M in 1,4-dioxane) were added, and finally the mixture was freeze-dried to provide the title product. Yield: 340 mg (86% of theory).

¹H-NMR (400 MHz, DMSO-d₆): δ [ppm]=8.57 (br. s, 3H), 8.29 (s, 1H), 7.93 (br. d, 1H), 6.89 (d, 1H), 4.40 (dt, 1H), 3.85 (s, 3H), 1.52 (d, 3H).

### Intermediate 179A

### Methyl 5-carbamoyl-1-[1-fluoro-2-(5-fluoro-2,3-dihydro-1,4-benzodioxin-6-yl)-2-oxoethyl]-1H-pyrazole-3-carboxylate

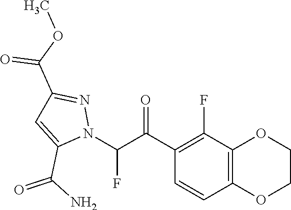 [SMILES: COC(=O)c1cc(C(N)=O)n(C(F)C(=O)c2ccc3c(c2F)OCCO3)n1]

A solution of methyl 6-(5-fluoro-2,3-dihydro-1,4-benzodioxin-6-yl)-4-oxo-4,5-dihydropyrazolo-[1,5-a]pyrazine-2-carboxylate (Intermediate 176A, 51.0 mg, 91% purity, 134 μmol) in acetonitrile (1.3 ml) was treated with Selectfluor® (50.1 mg, 95% purity, 134 μmol) at 0° C. After 30 min, the cooling bath was removed, and the reaction mixture was stirred at RT for 18 h. The mixture was then quenched with saturated aqueous sodium sulfite solution and partitioned between water and ethyl acetate. After extractive work-up with ethyl acetate (three times), the combined organic extracts were washed with brine, dried over sodium sulfate, filtered and evaporated to provide the title compound. Yield: 65.1 mg (91% of theory, 72% purity).

LC/MS [Method 7]: Rₜ=0.70 min; MS (ESIpos): m/z=364 [M+H]⁺.

### Intermediate 180A

### Ethyl 7-fluoro-6-(5-fluoro-2,3-dihydro-1,4-benzodioxin-6-yl)-4-oxo-4,5-dihydropyrazolo[1,5-a]-pyrazine-2-carboxylate

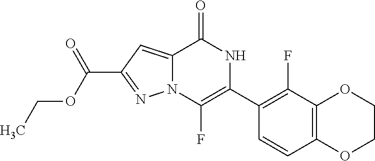 [SMILES: CCOC(=O)c1cc2c(=O)[nH]c(-c3ccc4c(c3F)OCCO4)c(F)n2n1]

To a solution of methyl 5-carbamoyl-1-[1-fluoro-2-(5-fluoro-2,3-dihydro-1,4-benzodioxin-6-yl)-2-oxoethyl]-1H-pyrazole-3-carboxylate (Intermediate 179A, 65.1 mg, 72% purity, 123 μmol) in ethanol (6.2 ml) were added Amberlyst® 15 (81.4 mg) and magnesium sulfate (163 mg, 1.35 mmol). The reaction mixture was refluxed overnight. After cooling to RT, the mixture was filtered through Celite, and the filtrate was evaporated. The residue was purified by preparative RP-HPLC (Chromatorex C-18, 125×30 mm; eluent: acetonitrile/water with 0.2% ammonia) to provide the title product. Yield: 16.1 mg (35% of theory).

LC/MS [Method 3]: Rₜ=1.53 min; MS (ESIpos): m/z=378 [M+H]⁺.

### Intermediate 181A

### 7-Fluoro-6-(5-fluoro-2,3-dihydro-1,4-benzodioxin-6-yl)-4-oxo-4,5-dihydropyrazolo[1,5-a]pyrazine-2-carboxylic acid

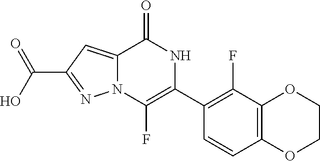 [SMILES: O=C(O)c1cc2c(=O)[nH]c(-c3ccc4c(c3F)OCCO4)c(F)n2n1]

A solution of ethyl 7-fluoro-6-(5-fluoro-2,3-dihydro-1,4-benzodioxin-6-yl)-4-oxo-4,5-dihydropyrazolo[1,5-a]pyrazine-2-carboxylate (Intermediate 180A, 16.0 mg, 42.4 μmol) in THF (1.0 ml) and water (250 μl) was treated with lithium hydroxide (4.06 mg, 170 μmol) and stirred at 40° C. for 18 h. After cooling, the THF was evaporated under reduced pressure, and to the remaining material water was added until complete dissolution. The solution was acidified with 1.0 M aqueous hydrochloric acid and subsequently freeze-dried. The material thus obtained was purified by chromatography on silica gel (eluent: 9:1 dichloromethane/methanol containing 10% acetic acid) to provide the title compound. Yield: 11.7 mg (74% of theory, 94% purity). LC/MS [Method 3]: Rₜ=0.99 min; MS (ESIpos): m/z=350 [M+H]⁺.

### Intermediate 182A

### 4-(4-Fluorophenyl)-1-methylpiperidin-4-ol

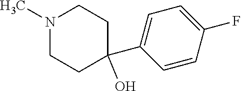

To (4-fluorophenyl)magnesium bromide (21 ml, 1.0 M solution in THF, 21 mmol) was added a solution of 1-methylpiperidin-4-one (2.2 ml, 18 mmol) in THF (80 ml) dropwise at 0° C. After 30 min, the cooling bath was removed, and stirring was continued at RT for 3 h. The reaction was then quenched with ice-cold sodium bicarbonate solution (50 ml), and the mixture was extracted with ethyl acetate (5×50 ml). The combined organic layers were washed with brine (2×20 ml), dried over magnesium sulfate, filtered and evaporated to afford the crude title compound which was used in the next step without further purification. Yield: 2.99 g (48% of theory, 60% purity).

LC/MS [Method 7]: Rₜ=0.24 min; MS (ESIpos): m/z=210 [M+H]⁺.

### Intermediate 183A

### N-[4-(4-Fluorophenyl)-1-methylpiperidin-4-yl]acetamide

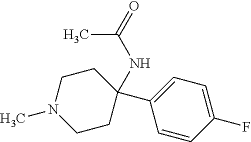 [SMILES: CC(=O)NC1(c2ccc(F)cc2)CCN(C)CC1]

Concentrated sulfuric acid (4.6 ml, 86 mmol) was added dropwise to a solution of 4-(4-fluoro-phenyl)-1-methylpiperidin-4-ol (Intermediate 182A, 3.01 g, 14.4 mmol) in acetonitrile (150 ml) at 0° C. The mixture was stirred at RT overnight. More concentrated sulfuric acid (2.3 ml, 43 mmol) was added, and stirring was continued at RT for 45 min. Then, water (150 ml) was added, and the mixture was washed with MTBE (2×100 ml). The organic layers were discarded, and the aqueous layer was brought to basic pH by addition of sodium hydroxide pellets. The aqueous layer was extracted with ethyl acetate (3×100 ml), and the combined organic layers were dried over magnesium sulfate, filtered and evaporated to afford the crude title compound which was used in the next step without further purification. Yield: 1.96 g (49% of theory, 89% purity).

LC/MS [Method 4]: Rₜ=1.03 min; MS (ESIpos): m/z=251 [M+H]⁺.

### Intermediate 184A

### 4-(4-Fluorophenyl)-1-methylpiperidin-4-amine

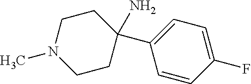

A mixture of N-[4-(4-fluorophenyl)-1-methylpiperidin-4-yl]acetamide (Intermediate 183A, 1.86 g, 7.43 mmol) with hydrochloric acid (35.0 ml, 6.0 M) was heated to reflux for 3 days. After cooling to RT, the mixture was poured into ice-water (250 ml), and the aqueous layer was brought to basic pH by addition of aqueous sodium hydroxide solution. The aqueous layer was extracted with ethyl acetate (1×100 ml, 3×50 ml), and the combined organic layers were dried over magnesium sulfate, filtered and evaporated to afford the crude title compound which was used in the next step without further purification. Yield: 1.25 g (40% of theory, 50% purity).

LC/MS [Method 4]: Rₜ=1.20 min; MS (ESIpos): m/z=209 [M+H]⁺.

### Intermediate 185A

### 6-(3-Chloro-4-methylphenyl)-7-methyl-4-oxo-4,5-dihydropyrazolo[1,5-a]pyrazine-2-carboxylic acid

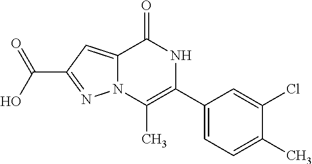 [SMILES: Cc1ccc(-c2[nH]c(=O)c3cc(C(=O)O)nn3c2C)cc1Cl]

Step A: Diethyl 1-[1-(3-chlor-4-methylphenyl)-1-oxopropan-2-yl]-1H-pyrazole-3,5-dicarboxylate Diethyl 1H-pyrazole-3,5-dicarboxylate (5.00 g, 23.3 mmol) was dissolved in acetone (100 ml), and 2-bromo-1-(3-chloro-4-methylphenyl)propan-1-one (6.10 g, 23.3 mmol), potassium carbonate (3.55 g, 25.7 mmol) and 3 drops of demineralized water were added. The mixture was stirred at RT overnight, after which LC/MS indicated complete conversion. The solids were filtered off, and the filtrate was concentrated under reduced pressure. The residue was partitioned between water and dichloromethane, and the phases were separated. The organic phase was washed with water and brine and dried over sodium sulfate. The solvent was removed under reduced pressure to give 10.2 g of the crude product which was directly used in the following step.

### Step B: Ethyl 6-(3-chloro-4-methylphenyl)-7-methyl-4-oxo-4,5-dihydropyrazolo[1,5-a]pyrazine-2-carboxylate

9.16 g of the crude product obtained above was dissolved in acetic acid (200 ml), and ammonium acetate (35.9 g, 466 mmol) was added. The mixture was refluxed overnight, after which LC/MS indicated complete conversion. The mixture was poured onto ice/water and neutralized by careful addition of sodium hydroxide. The precipitate formed was collected by filtration, washed with water and dichloromethane and dried under vacuum (20 mbar, 100° C.) to give 6.61 g of the ester product which was directly used in the following step.

LC/MS [Method 34]: Rₜ=1.20 min; MS (ESIpos): m/z=346 [M+H]⁺.

### Step C: 6-(3-Chloro-4-methylphenyl)-7-methyl-4-oxo-4,5-dihydropyrazolo[1,5-a]pyrazine-2-carboxylic acid

The ester obtained above (2.16 g, 6.25 mmol) was dissolved in water (80 ml) and ethanol (80 ml), and 1.0 M aqueous sodium hydroxide solution (50 ml, 50 mmol) was added. The suspension was sonicated for 50 min and then stirred at RT overnight. Thereafter, LC/MS indicated complete saponification. The ethanol was distilled off under reduced pressure, and the remaining solution was acidified to pH 2 with concentrated hydrochloric acid. The precipitated solid was collected by filtration, washed with water and dried under vacuum (20 mbar, 100° C.) to afford the title compound. Yield: 1.84 g (76% over three steps).

LC/MS [Method 34]: Rₜ=0.96 min; MS (ESIpos): m/z=318 [M+H]⁺.

### Intermediate 186A

### Ethyl 3-cyclopropyl-3-[(trifluoromethanesulfonyl)oxy]prop-2-enoate

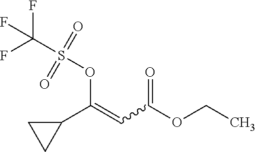 [SMILES: CCOC(=O)C=C(OS(=O)(=O)C(F)(F)F)C1CC1]

A solution of ethyl 3-cyclopropyl-3-oxopropanoate (10.0 g, 64.0 mmol) in toluene (400 ml) was cooled to 10° C. A solution of lithium hydroxide (11.5 g, 480 mmol) in water (120 ml) was added, and stirring was continued at 5° C. for 10 min. Then, trifluoromethanesulfonic anhydride (22 ml, 130 mmol) was slowly added dropwise while the temperature was kept below 25° C. After further stirring at RT for 2 h, the mixture was poured into water (400 ml) and extracted with ethyl acteate (three times). The combined organic layers were washed with water and brine, dried over sodium sulfate, filtered and evaporated to leave the crude title compound which was used in the next step without further purification. Yield: 11.7 g (63% of theory).

¹H-NMR (500 MHz, DMSO-d₆): δ [ppm]=6.05 (d, 1H), 4.13-4.07 (m, 2H), 2.12-2.05 (m, 1H), 1.21-1.17 (m, 3H), 0.95-0.89 (m, 4H).

### Intermediate 187A

### Diethyl 4-cyclopropyl-1H-pyrazole-3,5-dicarboxylate

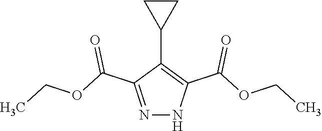 [SMILES: CCOC(=O)c1n[nH]c(C(=O)OCC)c1C1CC1]

A mixture of ethyl 3-cyclopropyl-3-[(trifluoromethanesulfonyl)oxy]prop-2-enoate (Intermediate 186A, 11.7 g, 40.6 mmol), ethyl diazoacetate (6.95 g, 60.9 mmol) and tetrakis(triphenylphosphine)palladium(0) (2.35 g, 2.03 mmol) in DMF (120 ml) was degassed by purging with argon for 5 min. Then, N-methylmorpholine (8.9 ml, 81 mmol) was added dropwise over 5 min. After complete addition, the reaction mixture was stirred at RT for 3 h and then at 60° C. overnight. After cooling to RT, the mixture was poured into water (500 ml) and extracted with ethyl acetate (three times). The combined organic layers were washed with water and brine, dried over sodium sulfate, filtered and evaporated. The residue was purified by silica gel column chromatography (eluent: cyclohexane/ethyl acetate) to afford the title compound. Yield: 3.50 g (32% of theory, 93% purity).

LC/MS [Method 3]: Rₜ=1.62 min; MS (ESIneg): m/z=251 [M−H]⁻.

¹H-NMR (500 MHz, DMSO-d₆): δ [ppm]=14.19 (br. s, 1H), 4.35-4.26 (m, 4H), 2.22-2.13 (m, 1H), 1.34-1.12 (m, 6H), 0.89-0.80 (m, 4H).

### Intermediate 188A

### 2-Bromo-1-[3-methyl-4-(trifluoromethyl)phenyl]ethan-1-one

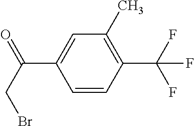

A solution of 1-[3-methyl-4-(trifluoromethyl)phenyl]ethan-1-one (3.00 g, 14.8 mmol) and phenyltrimethylammonium tribromide (5.58 g, 14.8 mmol) in THF (30 ml) was stirred at RT for 1 h. The insoluble material was filtered off, and the filter cake was washed with MTBE. The combined filtrates were evaporated under reduced pressure to give the crude title compound which was directly used in the next step without further purification. Yield: 6.90 g (99% of theory, 60% purity based on assumed quantitative conversion).

### Intermediate 189A

### Diethyl 4-cyclopropyl-1-{2-[3-methyl-4-(trifluoromethyl)phenyl]-2-oxoethyl}-1H-pyrazole-3,5-dicarboxylate

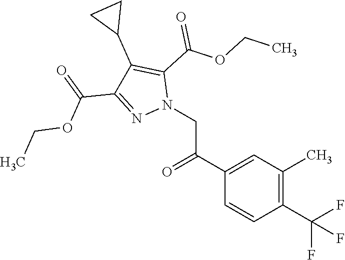 [SMILES: CCOC(=O)c1nn(CC(=O)c2ccc(C(F)(F)F)c(C)c2)c(C(=O)OCC)c1C1CC1]

A mixture of diethyl 4-cyclopropyl-1H-pyrazole-3,5-dicarboxylate (Intermediate 187A, 500 mg, 93% purity, 1.84 mmol), 2-bromo-1-[3-methyl-4-(trifluoromethyl)phenyl]ethan-1-one (Intermediate 188A, 1.04 g, 60% purity, 2.21 mmol) and potassium carbonate (637 mg, 4.61 mmol) in acetone (20 ml) was stirred at RT overnight. The insoluble material was filtered off, and the filtrate was concentrated under reduced pressure to give the crude title compound which was directly used in the next step without further purification. Yield: 1.20 g (quant., 70% purity based on assumed quantitative conversion).

LC/MS [Method 3]: Rₜ=2.41 min; MS (ESIpos): m/z=453 [M+H]⁺.

### Intermediate 190A

### Ethyl 3-cyclopropyl-6-[3-methyl-4-(trifluoromethyl)phenyl]-4-oxo-4,5-dihydropyrazolo[1,5-a]pyrazine-2-carboxylate

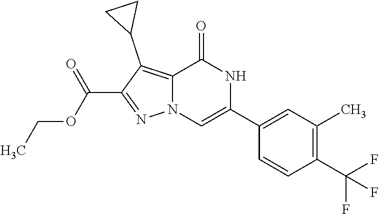 [SMILES: CCOC(=O)c1nn2cc(-c3ccc(C(F)(F)F)c(C)c3)[nH]c(=O)c2c1C1CC1]

A mixture of diethyl 4-cyclopropyl-1-{2-[3-methyl-4-(trifluoromethyl)phenyl]-2-oxoethyl}-1H-pyrazole-3,5-dicarboxylate (Intermediate 189A, 1.20 g, 70% purity, 1.86 mmol) and ammonium acetate (3.58 g, 46.4 mmol) in acetic acid (18 ml) was heated to 110° C. for 3 days. After cooling to RT, the reaction mixture was poured into water (700 ml). The precipitate was collected by filtration, washed with MTBE (200 ml) and dried to afford the title compound. Yield: 750 mg (quant.).

LC/MS [Method 3]: Rₜ=2.16 min; MS (ESIpos): m/z=406 [M+H]⁺.

¹H-NMR (500 MHz, DMSO-d₆): δ [ppm]=11.58 (s, 1H), 8.24 (d, 1H), 7.87 (s, 1H), 7.77-7.75 (m, 2H), 4.34 (q, 2H), 2.69-2.62 (m, 1H), 1.33 (t, 3H), 1.23-1.19 (m, 2H), 0.98-0.92 (m, 2H).

### Intermediate 191A

### 3-Cyclopropyl-6-[3-methyl-4-(trifluoromethyl)phenyl]-4-oxo-4,5-dihydropyrazolo[1,5-a]pyrazine-2-carboxylic acid

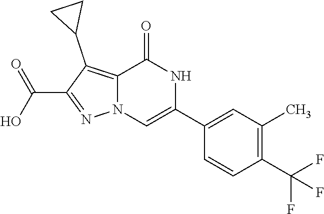 [SMILES: Cc1cc(-c2cn3nc(C(=O)O)c(C4CC4)c3c(=O)[nH]2)ccc1C(F)(F)F]

A mixture of ethyl 3-cyclopropyl-6-[3-methyl-4-(trifluoromethyl)phenyl]-4-oxo-4,5-dihydropyrazolo-[1,5-a]pyrazine-2-carboxylate (Intermediate 190A, 750 mg, 1.85 mmol) and lithium hydroxide (222 mg, 9.25 mmol) in methanol (22 ml) and water (4.5 ml) was heated to 50° C. for 2 h. After cooling to RT, the ethanol was distilled off under reduced pressure, and the residue was diluted with water and acidified by addition of 1.0 M hydrochloric acid. The precipitate was collected by filtration and dried to afford the title compound. Yield: 520 mg (74% of theory).

LC/MS [Method 3]: Rₜ=1.72 min; MS (ESIpos): m/z=378 [M+H]⁺.

¹H-NMR (500 MHz, DMSO-d₆): δ [ppm]=13.14 (br. s, 1H), 11.54 (s, 1H), 8.17 (s, 1H), 7.87 (s, 1H), 7.78-7.75 (m, 2H), 2.76-2.70 (m, 1H), 1.28-1.23 (m, 2H), 0.96-0.91 (m, 2H).

### Intermediate 192A

### 1-[4-Methyl-3-(trifluoromethyl)phenyl]ethanone

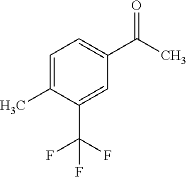

A solution of N-methoxy-N,4-dimethyl-3-(trifluoromethyl)benzamide (Intermediate 122A, 10.40 g, 42.1 mmol) in 100 ml THF was degassed with nitrogen three times. Then, methylmagnesium bromide solution (1.0 M in THF, 50.5 ml, 50.5 mmol) was added dropwise at 0° C. The resulting mixture was stirred at room temperature for 2 h. Water (100 ml) was then added, and the mixture was extracted with ethyl acetate. The combined organic layers were dried over anhydrous sodium sulfate, filtered and evaporated under reduced pressure to give 7.50 g (88% of theory, 89% purity) of the title compound.

LC/MS [Method 6]: Rₜ=1.13 min; MS (ESIpos): m/z=203 [M+H]⁺.

¹H-NMR (400 MHz, CDCl₃): δ [ppm]=8.19 (s, 1H), 8.00 (d, 1H), 7.39 (d, 1H), 2.61 (s, 3H), 2.54 (s, 3H).

¹⁹F-NMR (376 MHz, CDCl₃): δ [ppm]=−62.01 (s, 3F).

### Intermediate 193A

### 2,2-Dibromo-1-[4-methyl-3-(trifluoromethyl)phenyl]ethanone

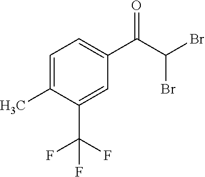 [SMILES: Cc1ccc(C(=O)C(Br)Br)cc1C(F)(F)F]

To a solution of 1-[4-methyl-3-(trifluoromethyl)phenyl]ethanone (Intermediate 192A, 7.30 g, 36.1 mmol) in 100 ml dichloromethane were added bromine (9.66 g, 54.2 mmol) and hydrobromic acid (4.1 ml, 36.1 mmol, 48% in water) dropwise. After stirring at RT overnight, the solvent was removed under reduce pressure, and the residue was purified by column chromatography on silica gel (eluent: petroleum ether/ethyl acetate 19:1) to give 12.00 g (92% of theory) of the title compound.

¹H-NMR (400 MHz, CDCl₃): δ [ppm]=8.34 (s, 1H), 8.17 (d, 1H), 7.45 (d, 1H), 6.61 (s, 1H), 2.59 (s, 3H).

¹⁹F-NMR (376 MHz, CDCl₃): δ [ppm]=−62.17 (s, 3F).

### Intermediate 194A

### 2-Bromo-1-[4-methyl-3-(trifluoromethyl)phenyl]ethanone

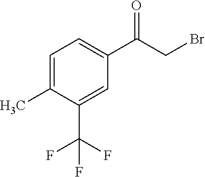

To a solution of 2,2-dibromo-1-[4-methyl-3-(trifluoromethyl)phenyl]ethanone (Intermediate 193A, 12.00 g, 33.3 mmol) in 100 ml THF at 0° C. were added triethylamine (5.7 ml, 56.7 mmol) and diethyl phosphonate (7.83 g, 56.7 mmol). After stirring at RT for 30 min, the solvent was removed under reduced pressure. The residue was diluted with water and extracted with ethyl acetate. The combined organic layers were dried with sodium sulfate, filtered and evaporated under reduced pressure to give 5.60 g (45% of theory, 75% purity) of the title compound, containing about 25% 1-[4-methyl-3-(trifluoromethyl)phenyl]ethanone. This material was used in the next step without further purification.

¹H-NMR (400 MHz, CDCl₃): δ [ppm]=8.22 (s, 1H), 8.01-8.05 (m, 1H), 7.43 (d, 1H), 4.42 (s, 2H), 2.54 (s, 3H).

¹⁹F-NMR (376 MHz, CDCl₃): δ [ppm]=−62.17 (s, 3F).

### Intermediate 195A

### Diethyl 1-{2-[4-methyl-3-(trifluoromethyl)phenyl]-2-oxoethyl}-1H-pyrazole-3,5-dicarboxylate

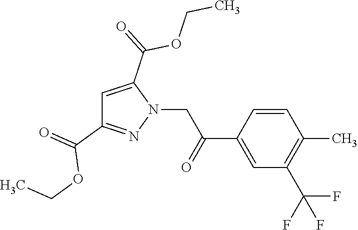 [SMILES: CCOC(=O)c1cc(C(=O)OCC)n(CC(=O)c2ccc(C)c(C(F)(F)F)c2)n1]

Diethyl 1H-pyrazole-3,5-dicarboxylate (3.00 g, 14.14 mmol), 2-bromo-1-[4-methyl-3-(trifluoro-methyl)phenyl]ethanone (Intermediate 194A, 5.56 g, 14.84 mmol, 75% purity) and potassium carbonate (2.05 g, 14.84 mmol) in 100 ml acetone were stirred at RT overnight. After filtering off the solids, the filtrate was evaporated and the residue partitioned between dichloromethane and water. The organic phase was washed with water and brine, then dried over sodium sulfate and concentrated. The crude product was purified by flash-chromatography on silica gel (120 g) eluting with 0-20% ethyl acetate in petroleum ether to give the title compound (5.60 g, 93% of theory, 97% purity).

LC/MS [Method 10]: Rₜ=1.26 min; MS (ESIpos): m/z=413 [M+H]⁺.

¹H-NMR (400 MHz, CDCl₃): δ [ppm]=8.20 (s, 1H), 8.01 (d, 1H), 7.47-7.45 (m, 2H), 6.09 (s, 2H), 4.43 (q, 2H), 4.29 (q, 2H), 2.58 (s, 3H), 1.41 (t, 3H), 1.33 (t, 3H).

¹⁹F-NMR (376 MHz, CDCl₃): δ [ppm]=−62.13 (s, 3F).

### Intermediate 196A

### Ethyl 6-[4-methyl-3-(trifluoromethyl)phenyl]-4-oxo-4,5-dihydropyrazolo[1,5-a]pyrazine-2-carboxylate

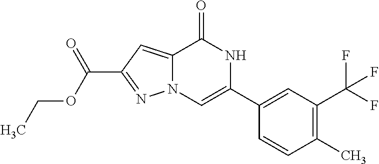 [SMILES: CCOC(=O)c1cc2c(=O)[nH]c(-c3ccc(C)c(C(F)(F)F)c3)cn2n1]

To a solution of diethyl 1-{2-[4-methyl-3-(trifluoromethyl)phenyl]-2-oxoethyl}-1H-pyrazole-3,5-dicarboxylate (Intermediate 195A, 5.60 g, 13.15 mmol, 97% purity) in 100 ml acetic acid, ammonium acetate (20.27 g, 262.99 mmol) was added, and the mixture was stirred at 110° C. overnight. The solution was then poured into ice-water and subsequently filtered. The solids were washed with water and dried in air to give the title compound (4.30 g, 90% of theory).

¹H-NMR (400 MHz, DMSO-d₆): δ [ppm]=11.93 (s, 1H), 8.33 (s, 1H), 8.05 (s, 1H), 7.94 (d, 1H), 7.58 (d, 1H), 7.42 (s, 1H), 4.35 (q, 2H), 2.50 (s, 3H), 1.33 (t, 3H).

¹⁹F-NMR (376 MHz, DMSO-d₆): δ [ppm]=−60.35 (s, 3F).

### Intermediate 197A

### 6-[4-Methyl-3-(trifluoromethyl)phenyl]-4-oxo-4,5-dihydropyrazolo[1,5-a]pyrazine-2-carboxylic acid

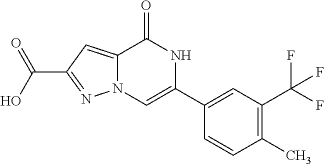 [SMILES: Cc1ccc(-c2cn3nc(C(=O)O)cc3c(=O)[nH]2)cc1C(F)(F)F]

To ethyl 6-[4-methyl-3-(trifluoromethyl)phenyl]-4-oxo-4,5-dihydropyrazolo[1,5-a]pyrazine-2-carboxylate (Intermediate 196A, 4.30 g, 11.77 mmol) in 40 ml ethanol, aqueous sodium hydroxide solution (3.0 M, 20 ml) was added, and the mixture was stirred at RT for 4 h. Then, the pH value of the aqueous phase was adjusted to 6 with diluted hydrochloric acid (3.0 M). The precipitate was filtered off, washed with water and dried in air to afford the title compound (3.77 g, 89% of theory, 93% purity).

LC/MS [Method 37]: Rₜ=3.41 min; MS (ESIpos): m/z=338 [M+H]⁺.

¹H-NMR (300 MHz, DMSO-d₆): δ [ppm]=13.28 (s, 1H), 11.91 (s, 1H), 8.29 (s, 1H), 8.05 (s, 1H), 7.93 (d, 1H), 7.57 (d, 1H), 7.37 (s, 1H), 2.52 (s, 3H).

¹⁹F-NMR (282 MHz, DMSO-d₆): δ [ppm]=−60.31 (s, 3F).

### Intermediate 198A

### Diethyl 1-{2-[3-methyl-4-(trifluoromethyl)phenyl]-2-oxoethyl}-1H-pyrazole-3,5-dicarboxylate

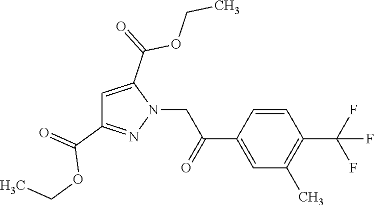 [SMILES: CCOC(=O)c1cc(C(=O)OCC)n(CC(=O)c2ccc(C(F)(F)F)c(C)c2)n1]

A mixture of diethyl 1H-pyrazole-3,5-dicarboxylate (3.00 g, 14.1 mmol), 2-bromo-1-[3-methyl-4-(trifluoromethyl)phenyl]ethanone (Intermediate 188A, 4.37 g, 15.5 mmol) and potassium carbonate (2.15 g, 15.5 mmol) in acetone (40 ml) was stirred at RT overnight. After filtering off the solids, the filtrate was evaporated and the residue partitioned between dichloromethane and water. The organic phase was washed with water and brine, then dried over sodium sulfate and evaporated to give the title compound (5.70 g, 81% of theory, 83% purity).

LC/MS [Method 6]: Rₜ=1.29 min; MS (ESIpos): m/z=413 [M+H]⁺.

¹H-NMR (400 MHz, DMSO-d₆): δ [ppm]=8.11 (s, 1H), 8.04 (d, 1H), 7.90 (d, 1H), 7.36 (s, 1H), 6.30 (s, 2H), 4.31 (q, 2H), 4.20 (q, 2H), 2.55 (s, 3H), 1.31 (t, 3H), 1.19 (t, 3H).

¹⁹F-NMR (376 MHz, DMSO-d₆): δ [ppm]=−60.95 (s, 3F).

### Intermediate 199A

### Ethyl 6-[3-methyl-4-(trifluoromethyl)phenyl]-4-oxo-4,5-dihydropyrazolo[1,5-a]pyrazine-2-carboxylate

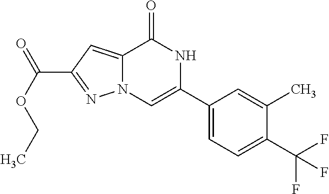 [SMILES: CCOC(=O)c1cc2c(=O)[nH]c(-c3ccc(C(F)(F)F)c(C)c3)cn2n1]

Ammonium acetate (21.3 g, 276.5 mmol) was added to a solution of diethyl 1-{2-[3-methyl-4-(trifluoromethyl)phenyl]-2-oxoethyl}-1H-pyrazole-3,5-dicarboxylate (Intermediate 198A, 5.70 g, 13.8 mmol) in acetic acid (100 ml). After stirring at 110° C. overnight, the solution was poured into ice-water and then filtered. The filter cake was washed with water and dried in air to give the title compound (4.88 g, 97% of theory).

¹H-NMR (300 MHz, DMSO-d₆): δ [ppm]=11.88 (s, 1H), 8.35 (s, 1H), 7.88 (s, 1H), 7.79-7.77 (m, 2H), 7.44 (s, 1H), 4.36 (q, 2H), 2.50 (s, 3H), 1.34 (t, 3H).

¹⁹F-NMR (282 MHz, DMSO-d₆): δ [ppm]=−60.31 (s, 3F).

### Intermediate 200A

### 6-[3-Methyl-4-(trifluoromethyl)phenyl]-4-oxo-4,5-dihydropyrazolo[1,5-a]pyrazine-2-carboxylic acid

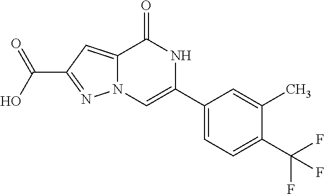 [SMILES: Cc1cc(-c2cn3nc(C(=O)O)cc3c(=O)[nH]2)ccc1C(F)(F)F]

Aqueous sodium hydroxide solution (20 ml, 3.0 M) was added to a solution of ethyl 6-[3-methyl-4-(trifluoromethyl)phenyl]-4-oxo-4,5-dihydropyrazolo[1,5-a]pyrazine-2-carboxylate (Intermediate 199A, 4.88 g, 13.4 mmol) in ethanol (40 ml), and the mixture was stirred at RT for 4 h. The pH value of the aqueous phase was adjusted to 6 with diluted hydrochloric acid (3 M). Precipitated solids were filtered off and washed with water to afford the title compound (3.01 g, 66% of theory).

LC/MS [Method 30]: Rₜ=1.24 min, MS (ESIpos): m/z=338 [M+H]⁺.

¹H-NMR (400 MHz, DMSO-d₆): δ [ppm]=13.31 (s, 1H), 11.85 (s, 1H), 8.30 (s, 1H), 7.88 (s, 1H), 7.80-7.75 (m, 2H), 7.39 (s, 1H), 2.51 (s, 3H).

¹⁹F-NMR (376 MHz, DMSO-d₆): δ [ppm]=−60.15 (s, 3F).

### Intermediate 201A

### 7-Bromo-N-hydroxy-3,4-dihydronaphthalen-1(2H)-imine

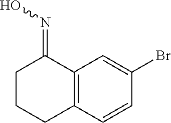 [SMILES: ON=C1CCCc2ccc(Br)cc21]

A mixture of 7-bromo-3,4-dihydronaphthalen-1(2H)-one (40.0 g, 178 mmol), hydroxylamine hydrochloride (37.0 g, 533 mmol) and sodium acetate (45.2 g, 551 mmol) in 200 ml ethanol was stirred at 80° C. for 2 h. After cooling to room temperature, 100 ml water was added, and the ethanol was removed under reduced pressure. The solid was collected by filtration, washed with water and dried in the oven to give 41.0 g (93% of theory, 97% purity) of the title compound. LC/MS [Method 6]: Rₜ=1.11 min; MS (ESIpos): m/z=240 [M+H]⁺.

¹H-NMR (400 MHz, CDCl₃): δ [ppm]=8.06 (d, 1H), 7.37 (dd, 1H), 7.03 (d, 1H), 2.79 (t, 2H), 2.71 (t, 2H), 1.89-1.83 (m, 2H).

### Intermediate 202A

### 7-Bromonaphthalen-1-amine and 8-bromo-2-methyl-4,5-dihydronaphtho[1,2-d][1,3]oxazole

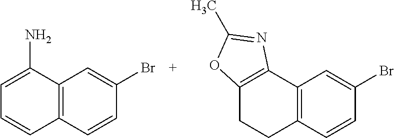

Concentrated sulfuric acid (37.9 ml, 711 mmol) was added to a mixture of 7-bromo-N-hydroxy-3,4-dihydronaphthalen-1(2H)-imine (Intermediate 201A, 35.0 g, 142 mmol) and acetic anhydride (40.2 ml, 426 mmol) in acetic acid (100 ml) at room temperature. The resulting mixture was stirred at 60° C. for 24 h. After cooling to room temperature, the reaction mixture was quenched with water (100 ml), the pH was adjusted to 13 with 6 M aqueous sodium hydroxide solution, and the mixture was extracted with ethyl acetate. The combined organic layers were dried over anhydrous magnesium sulfate, filtered and concentrated under reduced pressure. The residue was purified by flash-chromatography on silica gel (eluent: petroleum ether/ethyl acetate 19:1) to give two product fractions.

Fraction 1: 14.00 g (44% of theory, 99% purity) of 7-bromonaphthalen-1-amine.

LC/MS [Method 6]: Rₜ=1.08 min; MS (ESIpos): m/z=222 [M+H]⁺.

¹H-NMR (300 MHz, DMSO-d₆): δ [ppm]=8.33 (s, 1H), 7.69 (d, 1H), 7.49 (dd, 1H), 7.23 (t, 1H), 7.08 (d, 1H), 6.70 (d, 1H), 5.83 (s, 2H).

Fraction 2: 6.00 g (16% of theory, 95% purity) of 8-bromo-2-methyl-4,5-dihydronaphtho[1,2-d]-[1,3]oxazole.

LC/MS [Method 30]: Rₜ=1.62 min; MS (ESIpos): m/z=264 [M+H]⁺.

¹H-NMR (300 MHz, DMSO-d₆): δ [ppm]=7.49 (s, 1H), 7.32 (d, 1H), 7.20 (d, 1H), 3.10-3.04 (t, 2H), 2.97-2.91 (m, 2H), 2.46 (s, 3H).

### Intermediate 203A

### 7-Bromo-1-fluoronaphthalene

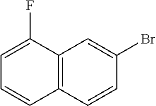 [SMILES: Fc1cccc2ccc(Br)cc12]

To a solution of boron trifluoride diethyl etherate (23.73 g, 47% in diethyl ether, 78.6 mmol) in 40 ml 1,2-dimethoxyethane was added, at −5° C., a solution of 8-amino-2-bromo-naphthalene (Intermediate 202A/Fraction 1, 16.00 g, 72.0 mmol) in 30 ml 1,2-dimethoxyethane dropwise over 30 min. After one hour, a solution of tert.-butylnitrite (7.09 g, 68.8 mmol) in 30 ml 1,2-dimethoxy-ethane was added, and the mixture was stirred at RT for 2 h. The solvent was then removed under reduced pressure. The residue was dissolved in 60 ml o-xylene and heated to reflux for 50 min. After cooling to room temperature, the mixture was concentrated. The residue was dissolved in dichloromethane and washed with aqueous sodium hydrogencarbonate solution. The organic phase was dried over anhydrous sodium sulfate, filtered and concentrated under reduced pressure. The residue was purified by flash-chromatography on silica gel (eluent: petroleum ether 100%) to give 9.48 g (58% of theory) of the title compound.

¹H-NMR (400 MHz, CDCl₃): δ [ppm]=8.27 (s, 1H), 7.73 (d, 1H), 7.60 (d, 2H), 7.45-7.39 (m, 1H), 7.20-7.15 (m, 1H).

¹⁹F-NMR (376 MHz, CDCl₃): δ [ppm]=−122.94 (s, 1F).

### Intermediate 204A

### 1-(8-Fluoro-2-naphthyl)ethanone

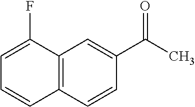

n-Butyllithium (14.9 ml, 2.5 M in THF, 37.3 mmol) was added to a solution of 7-bromo-1-fluoro-naphthalene (Intermediate 203A, 8.00 g, 35.6 mmol) in 100 ml THF under nitrogen at −70° C., and the solution was kept stirring at this temperature for 1 h. Then, N-methoxy-N-methylacetamide (4.03 g, 39.1 mmol) was added. After stirring at −70° C. for 20 min, water (20 ml) was added, and the resulting mixture was extracted with ethyl acetate. The combined organic layers were dried over anhydrous sodium sulfate, filtered and evaporated under reduced pressure to give 6.37 g (95% of theory) of the title compound.

¹H-NMR (400 MHz, CDCl₃): δ [ppm]=8.71 (s, 1H), 8.10 (d, 1H), 7.91 (d, 1H), 7.67 (d, 1H), 7.56-7.51 (m, 1H), 7.26-7.21 (m, 1H), 2.73 (s, 3H).

¹⁹F-NMR (376 MHz, CDCl₃): δ [ppm]=−121.22 (s, 1F).

### Intermediate 205A

### 2-Bromo-1-(8-fluoro-2-naphthyl)ethanone

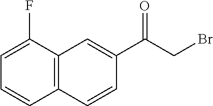 [SMILES: O=C(CBr)c1ccc2cccc(F)c2c1]

1-(8-Fluoro-2-naphthyl)ethanone (Intermediate 204A, 6.00 g, 31.9 mmol) and phenyltrimethylammonium tribromide (11.98 g, 31.9 mmol) in 150 ml dichloromethane were stirred at RT for 4 h. The reaction mixture was then concentrated, and the residue was purified by flash-chromatography on silica gel (200 g; eluent: 0-2% ethyl acetate in petroleum ether) to give the title compound (7.7 g, 83% of theory, 92% purity).

¹H-NMR (400 MHz, CDCl₃): δ [ppm]=8.75 (s, 1H), 8.09 (d, 1H), 7.94 (d, 1H), 7.68 (d, 1H), 7.54-7.59 (m, 1H), 7.26-7.23 (m, 1H), 4.60 (s, 2H).

¹⁹F-NMR (376 MHz, CDCl₃): δ [ppm]=−120.66 (s, 1F).

### Intermediate 206A

### Diethyl 1-[2-(8-fluoro-2-naphthyl)-2-oxoethyl]-1H-pyrazole-3,5-dicarboxylate

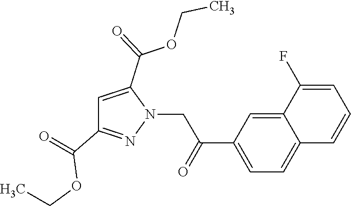 [SMILES: CCOC(=O)c1cc(C(=O)OCC)n(CC(=O)c2ccc3cccc(F)c3c2)n1]

A mixture of diethyl 1H-pyrazole-3,5-dicarboxylate (3.00 g, 14.1 mmol), 2-bromo-1-(8-fluoronaphthalen-2-yl)ethanone (Intermediate 205A, 3.78 g, 14.1 mmol) and potassium carbonate (2.15 g, 15.6 mmol) in acetone (40 ml) was stirred at RT overnight. After filtering off the solids, the filtrate was evaporated and the residue partitioned between dichloromethane and water. The organic phase was washed with water and brine, then dried over sodium sulfate and concentrated. The crude product was purified by flash-chromatography on silica gel (80 g, eluent: 0-20% ethyl acetate in petroleum ether) to give the title compound (4.21 g, 70% of theory, 94% purity).

LC/MS [Method 6]: Rₜ=1.25 min; MS (ESIpos): m/z=399 [M+H]⁺.

¹H-NMR (400 MHz, DMSO-d₆): δ [ppm]=8.85 (s, 1H), 8.22 (d, 1H), 8.14-8.10 (m, 1H), 7.94 (d, 1H), 7.77-7.72 (m, 1H), 7.55-7.50 (m, 1H), 7.39 (s, 1H), 6.46 (s, 2H), 4.33 (q, 2H), 4.24 (q, 2H), 1.32 (t, 3H), 1.18 (t, 3H).

### Intermediate 207A

### Ethyl 6-(8-fluoro-2-naphthyl)-4-oxo-4,5-dihydropyrazolo[1,5-a]pyrazine-2-carboxylate

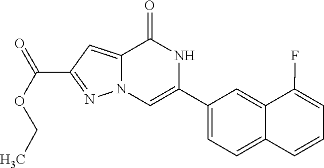 [SMILES: CCOC(=O)c1cc2c(=O)[nH]c(-c3ccc4cccc(F)c4c3)cn2n1]

Ammonium acetate (16.29 g, 211.35 mmol) was added to a solution of diethyl 1-[2-(8-fluoro-2-naphthyl)-2-oxoethyl]-1H-pyrazole-3,5-dicarboxylate (Intermediate 206A, 4.21 g, 10.57 mmol) in acetic acid (50 ml), and the mixture was stirred at 110° C. overnight. After cooling to RT, the solution was poured into ice-water, and the precipitate was filtered off and washed with water. The solid was dried in air to give 3.5 g (48% of theory, 51% purity) of the title compound which contained about 23% ester hydrolysis product 6-(8-fluoro-2-naphthyl)-4-oxo-4,5-dihydropyrazolo[1,5-a]-pyrazine-2-carboxylic acid. This material was used for the next step without further purification.

LC/MS [Method 6]: Rₜ=1.08 min; MS (ESIneg): m/z=350 [M−H]⁻.

### Intermediate 208A

### 6-(8-Fluoro-2-naphthyl)-4-oxo-4,5-dihydropyrazolo[1,5-a]pyrazine-2-carboxylic acid

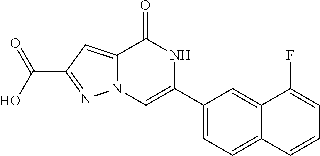 [SMILES: O=C(O)c1cc2c(=O)[nH]c(-c3ccc4cccc(F)c4c3)cn2n1]

A solution of ethyl 6-(8-fluoro-2-naphthyl)-4-oxo-4,5-dihydropyrazolo[1,5-a]pyrazine-2-carboxylate (Intermediate 207A, 3.5 g, 7.37 mmol, 51% purity, containing 23% desired ester hydrolysis product) in 20 ml ethanol was stirred with aqueous sodium hydroxide solution (15 ml, 3.0 M) at RT for 4 h. Then, the pH value of the aqueous phase was adjusted to 6 with diluted hydrochloric acid (3 M). The precipitated solids were isolated by filtration, washed with water and dried in air to afford the title compound (2.19 g, 79% of theory, 85% purity).

¹H-NMR (300 MHz, DMSO-d₆): δ [ppm]=13.28 (br. s, 1H), 12.03 (s, 1H), 8.51 (s, 1H), 8.41 (s, 1H), 8.13 (d, 1H), 8.00 (d, 1H), 7.86 (d, 1H), 7.64-7.57 (m, 1H), 7.47-7.43 (m, 1H), 7.41 (s, 1H).

### Intermediate 209A

### rac-2-Bromo-1-(3,4-dihydro-2H-chromen-2-yl)ethanone

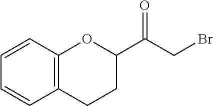 [SMILES: O=C(CBr)C1CCc2ccccc2O1]

A solution of chromane-2-carboxylic acid (0.20 g, 1.1 mmol) in thionyl chloride (5.0 ml) was stirred at 80° C. for 2 h. After cooling to room temperature, the thionyl chloride was removed under reduced pressure to give a solid which was dissolved in 10 ml THF and 10 ml acetonitrile. Then, [(trimethylsilyl)methyl]diazene (1.1 ml, 2.0 M solution in hexane, 2.2 mmol) was added to the stirred solution. After 1 h, 40% aqueous hydrobromic acid solution (0.23 ml) was added at 0° C. The resulting mixture was stirred for further 15 min. Then, saturated aqueous sodium bicarbonate solution was added carefully to adjust the pH value to above 7. The layers were separated, and the organic layer was dried over anhydrous sodium sulfate, filtered and concentrated under reduced pressure. The residue was purified by flash-chromatography on silica gel (eluent: petroleum ether/ethyl acetate 19:1) to give the title compound (0.19 g, 66% of theory).

¹H-NMR (400 MHz, CDCl₃): δ [ppm]=7.17-7.05 (m, 2H), 6.95-6.85 (m, 2H), 4.76-4.72 (m, 1H), 4.42 (d, 1H), 4.26 (d, 1H), 2.90-2.73 (m, 2H), 2.37-2.29 (m, 1H), 2.15-2.05 (m, 1H).

### Intermediate 210A

### rac-Diethyl 1-[2-(3,4-dihydro-2H-chromen-2-yl)-2-oxoethyl]-1H-pyrazole-3,5-dicarboxylate

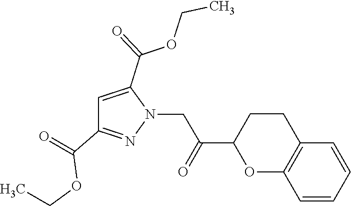 [SMILES: CCOC(=O)c1cc(C(=O)OCC)n(CC(=O)C2CCc3ccccc3O2)n1]

A mixture of diethyl 1H-pyrazole-3,5-dicarboxylate (2.00 g, 9.42 mmol), 2-bromo-1-(3,4-dihydro-2H-chromen-2-yl)ethanone (Intermediate 209A, 2.52 g, 9.90 mmol) and potassium carbonate (1.43 g, 10.4 mmol) in dry acetone (50 ml) was stirred at RT overnight. After filtering off the solids, the filtrate was evaporated and the residue partitioned between dichloromethane and water. The organic phase was washed with water and brine, then dried over sodium sulfate and concentrated. The residue was purified by flash-chromatography on silica gel (80 g, eluent: 0-25% ethyl acetate in petroleum ether) to give the title compound (2.88 g, 79% of theory).

¹H-NMR (300 MHz, CDCl₃): δ [ppm]=7.44 (s, 1H), 7.21-7.16 (m, 1H), 7.12-7.10 (m, 1H), 6.95-6.90 (m, 2H), 5.95 (d, 1H), 5.69 (d, 1H), 4.75-4.71 (m, 1H), 4.42 (q, 2H), 4.30 (q, 2H), 2.88-2.82 (m, 2H), 2.36-2.17 (m, 2H), 1.40 (t, 3H), 1.35 (t, 3H).

### Intermediate 211A

### rac-Ethyl 6-(3,4-dihydro-2H-chromen-2-yl)-4-oxo-4,5-dihydropyrazolo[1,5-a]pyrazine-2-carboxylate

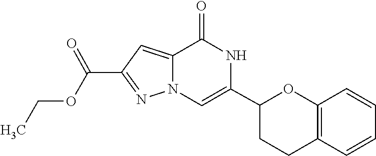 [SMILES: CCOC(=O)c1cc2c(=O)[nH]c(C3CCc4ccccc4O3)cn2n1]

A mixture of diethyl 1-[2-(3,4-dihydro-2H-chromen-2-yl)-2-oxoethyl]-1H-pyrazole-3,5-dicarboxylate (Intermediate 210A, 2.88 g, 7.45 mmol) and ammonium acetate (11.49 g, 149.1 mmol) in acetic acid (50 ml) was stirred at 110° C. overnight. The solution was then poured into ice-water and filtered. The filter cake was washed with water and dried in air to give the title compound (1.58 g, 63% of theory).

¹H-NMR (400 MHz, DMSO-d₆): δ [ppm]=11.74 (s, 1H), 7.85 (s, 1H), 7.39 (s, 1H), 7.14-7.10 (m, 2H), 6.90-6.85 (m, 2H), 5.01-4.98 (m, 1H), 4.33 (q, 2H), 2.93-2.75 (m, 2H), 2.26-2.20 (m, 2H), 1.32 (t, 3H).

### Intermediate 212A

### rac-6-(3,4-Dihydro-2H-chromen-2-yl)-4-oxo-4,5-dihydropyrazolo[1,5-a]pyrazine-2-carboxylic acid

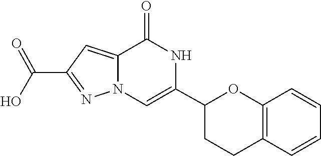 [SMILES: O=C(O)c1cc2c(=O)[nH]c(C3CCc4ccccc4O3)cn2n1]

Aqueous sodium hydroxide solution (5.0 ml, 3.0 M) was added to a solution of ethyl 6-(3,4-dihydro-2H-chromen-2-yl)-4-oxo-4,5-dihydropyrazolo[1,5-a]pyrazine-2-carboxylate (Intermediate 211A, 1.50 g, 4.42 mmol) in 10 ml ethanol, and the mixture was stirred at RT for 2 h. The pH value of the aqueous phase was adjusted to 6 with diluted hydrochloric acid (3 M). The solids were isolated by filtration, washed with water and dried in air yielding 1.10 g of the crude title compound.

150 mg of this material were purified by preparative HPLC (column: XBridge prep C18, 10 μm, 19×250 mm; mobile phase A: water with 10 mM ammonium bicarbonate, mobile phase B: acetonitrile; gradient: 35% B to 40% B in 8 min; detection: 254/220 nm) to give the title compound (51 mg, 98% purity, analytical data below). Another 950 mg of the crude product were washed with methanol and water to give 507 mg of the title compound with 86% purity (combined yield: 35% of theory).

LC/MS [Method 38]: Rₜ=2.28 min; MS (ESIpos): m/z=312 [M+H]⁺.

¹H-NMR (400 MHz, DMSO-d₆): δ [ppm]=13.28 (br. s, 1H), 11.69 (s, 1H), 7.81 (s, 1H), 7.33 (s, 1H), 7.13-7.10 (m, 2H), 6.90-6.86 (m, 2H), 5.01-4.98 (m, 1H), 2.95-2.77 (m, 2H), 2.33-2.20 (m, 2H).

### Intermediate 213A

### Diethyl 1-[2-(3-fluoro-4-methylphenyl)-2-oxoethyl]-4-(trifluoromethyl)-1H-pyrazole-3,5-dicarboxylate

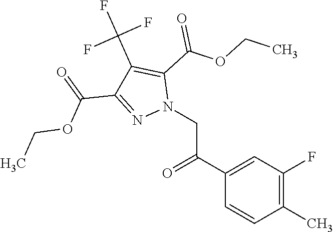 [SMILES: CCOC(=O)c1nn(CC(=O)c2ccc(C)c(F)c2)c(C(=O)OCC)c1C(F)(F)F]

A mixture of diethyl 4-(trifluoromethyl)-1H-pyrazole-3,5-dicarboxylate (Intermediate 32A, 505 mg, 1.80 mmol), 2-bromo-1-(3-fluoro-4-methylphenyl)ethan-1-one (500 mg, 2.16 mmol) and potassium carbonate (623 mg, 4.51 mmol) in acetone (16 ml) was stirred at RT for 3 h. The insoluble materials were filtered off and washed with acetone. The filtrate was concentrated and dried under reduced pressure to afford the crude title compound (918 mg, 75% of theory, 64% purity) which was used in the next step without further purification.

LC/MS [Method 3]: Rₜ=2.26 min; MS (ESIpos): m/z=431 [M+H]⁺.

### Intermediate 214A

### Ethyl 6-(3-fluoro-4-methylphenyl)-4-oxo-3-(trifluoromethyl)-4,5-dihydropyrazolo[1,5-a]pyrazine-2-carboxylate

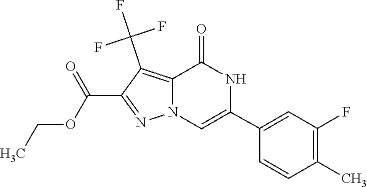 [SMILES: CCOC(=O)c1nn2cc(-c3ccc(C)c(F)c3)[nH]c(=O)c2c1C(F)(F)F]

A mixture of diethyl 1-[2-(3-fluoro-4-methylphenyl)-2-oxoethyl]-4-(trifluoromethyl)-1H-pyrazole-3,5-dicarboxylate (Intermediate 213A, 987 mg, 64% purity, 1.46 mmol) and ammonium acetate (4.49 g, 58.3 mmol) in acetic acid (29 ml) was heated to 110° C. for 3 d. After cooling to RT, the mixture was poured into water (400 ml) and the precipitate was collected by filtration, washed with ethyl acetate (10 ml) and MTBE (50 ml) and dried to afford the title compound (628 mg, 96% of theory, 85% purity) which was used in the next step without further purification.

LC/MS [Method 3]: Rₜ=1.96 min; MS (ESIneg): m/z=382 [M−H]⁻.

¹H-NMR (500 MHz, DMSO-d₆): δ [ppm]=12.12 (s, 1H), 8.32 (s, 1H), 7.62 (dd, 1H), 7.57-7.51 (m, 1H), 7.47-7.39 (m, 1H), 4.39 (q, 2H), 2.29 (s, 3H), 1.32 (t, 3H).

### Intermediate 215A

### 6-(3-Fluoro-4-methylphenyl)-4-oxo-3-(trifluoromethyl)-4,5-dihydropyrazolo[1,5-a]pyrazine-2-carboxylic acid

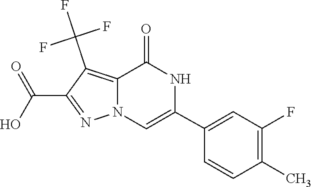 [SMILES: Cc1ccc(-c2cn3nc(C(=O)O)c(C(F)(F)F)c3c(=O)[nH]2)cc1F]

A mixture of ethyl 6-(3-fluoro-4-methylphenyl)-4-oxo-3-(trifluoromethyl)-4,5-dihydropyrazolo-[1,5-a]pyrazine-2-carboxylate (Intermediate 214A, 628 mg, 1.64 mmol) and lithium hydroxide (392 mg, 16.4 mmol) in ethanol (6.6 ml) and water (3.3 ml) was stirred at RT for 4 h. The ethanol was removed under reduced pressure and the resulting aqueous phase was adjusted to pH 2 by addition of 1.0 M hydrochloric acid. The precipitate was collected by filtration and dried to afford the title compound (531 mg, 80% of theory, 88% purity) which was used in the next step without further purification.

LC/MS [Method 3]: Rₜ=1.27 min; MS (ESIpos): m/z=356 [M+H]⁺.

¹H-NMR (500 MHz, DMSO-d₆): δ [ppm]=14.05 (br s, 1H), 12.08 (br s, 1H), 8.28 (s, 1H), 7.64-7.59 (m, 1H), 7.56-7.52 (m, 1H), 7.48-7.39 (m, 1H), 2.29 (s, 3H).

### Intermediate 216A

### Diethyl 1-[2-(3-chloro-4-methylphenyl)-2-oxoethyl]-4-methyl-1H-pyrazole-3,5-dicarboxylate

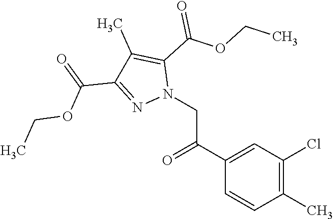 [SMILES: CCOC(=O)c1nn(CC(=O)c2ccc(C)c(Cl)c2)c(C(=O)OCC)c1C]

A mixture of diethyl 4-methyl-1H-pyrazole-3,5-dicarboxylate (Intermediate 54A, 600 mg, 2.65 mmol), 2-bromo-1-(3-chloro-4-methylphenyl)ethan-1-one (1.13 g, 70% purity, 3.18 mmol) and potassium carbonate (916 mg, 6.63 mmol) in acetone (23 ml) was stirred at RT overnight. The insoluble materials were filtered off and washed with acetone. The filtrate was concentrated and dried under reduced pressure to afford the crude title compound (1.25 g, 98% of theory, 82% purity) which was used in the next step without further purification.

LC/MS [Method 3]: Rₜ=2.28 min; MS (ESIpos): m/z=393 [M+H]⁺.

### Intermediate 217A

### Ethyl 6-(3-chloro-4-methylphenyl)-3-methyl-4-oxo-4,5-dihydropyrazolo[1,5-a]pyrazine-2-carboxylate

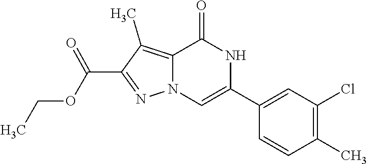 [SMILES: CCOC(=O)c1nn2cc(-c3ccc(C)c(Cl)c3)[nH]c(=O)c2c1C]

A mixture of diethyl 1-[2-(3-chloro-4-methylphenyl)-2-oxoethyl]-4-methyl-1H-pyrazole-3,5-dicarboxylate (Intermediate 216A, 1.24 g, 82% purity, 2.59 mmol) and ammonium acetate (7.98 g, 104 mmol) in acetic acid (52 ml) was stirred at 110° C. overnight. After cooling to RT, the mixture was poured into water (600 ml) and the precipitate was collected by filtration and dried to afford the title compound (890 mg, 99% of theory) which was used in the next step without further purification.

LC/MS [Method 3]: 1.99 min; MS (ESIneg): m/z=344 [M−H]⁻.

¹H-NMR (400 MHz, DMSO-d₆): δ [ppm]=11.57 (br s, 1H), 8.16 (s, 1H), 7.84 (d, 1H), 7.63 (dd, 1H), 7.47 (d, 1H), 4.34 (q, 2H), 2.64 (s, 3H), 2.38 (s, 3H), 1.33 (t, 3H).

### Intermediate 218A

### 6-(3-Chloro-4-methylphenyl)-3-methyl-4-oxo-4,5-dihydropyrazolo[1,5-a]pyrazine-2-carboxylic acid

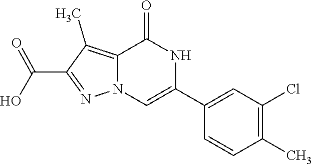 [SMILES: Cc1ccc(-c2cn3nc(C(=O)O)c(C)c3c(=O)[nH]2)cc1Cl]

A mixture of ethyl 6-(3-chloro-4-methylphenyl)-3-methyl-4-oxo-4,5-dihydropyrazolo[1,5-a]-pyrazine-2-carboxylate (Intermediate 217A, 890 mg, 2.57 mmol) and lithium hydroxide (616 mg, 25.7 mmol) in methanol (100 ml) and water (25 ml) was stirred at RT for 4 h. The methanol was removed under reduced pressure and the resulting aqueous phase was adjusted to pH 2 by addition of 1.0 M hydrochloric acid. The precipitate was collected by filtration and dried to afford the title compound (700 mg, 86% of theory).

LC/MS [Method 7]: Rₜ=0.81 min; MS (ESIneg): m/z=316 [M−H]⁻.

¹H-NMR (400 MHz, DMSO-d₆): δ [ppm]=13.08 (br s, 1H), 11.52 (s, 1H), 8.10 (s, 1H), 7.84 (d, 1H), 7.63 (dd, 1H), 7.46 (d, 1H), 2.63 (s, 3H), 2.38 (s, 3H).

### Intermediate 219A

### Diethyl 1-[2-(3-chloro-4-methylphenyl)-2-oxoethyl]-4-(trifluoromethyl)-1H-pyrazole-3,5-dicarboxylate

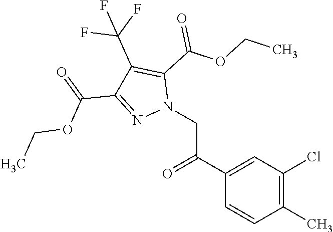 [SMILES: CCOC(=O)c1nn(CC(=O)c2ccc(C)c(Cl)c2)c(C(=O)OCC)c1C(F)(F)F]

A mixture of diethyl 4-(trifluoromethyl)-1H-pyrazole-3,5-dicarboxylate (Intermediate 32A, 600 mg, 2.14 mmol), 2-bromo-1-(3-chloro-4-methylphenyl)ethan-1-one (909 mg, 70% purity, 2.57 mmol) and potassium carbonate (740 mg, 5.35 mmol) in acetone (19 ml) was stirred at RT overnight. The insoluble materials were filtered off and washed with acetone. The filtrate was concentrated and dried under reduced pressure to afford the crude title compound (1.20 g, 99% of theory, 79% purity) which was used in the next step without further purification.

LC/MS [Method 3]: Rₜ=2.37 min; MS (ESIpos): m/z=447 [M+H]⁺.

¹H-NMR (400 MHz, DMSO-d₆): δ [ppm]=8.06 (d, 1H), 7.91 (dd, 1H), 7.61 (d, 1H), 6.27 (s, 2H), 4.36 (q, 2H), 4.26 (q, 2H), 2.45 (s, 3H), 1.32-1.28 (m, 3H), 1.13 (t, 3H).

### Intermediate 220A

### Ethyl 6-(3-chloro-4-methylphenyl)-4-oxo-3-(trifluoromethyl)-4,5-dihydropyrazolo[1,5-a]pyrazine-2-carboxylate

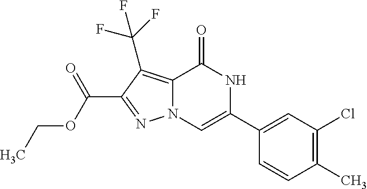 [SMILES: CCOC(=O)c1nn2cc(-c3ccc(C)c(Cl)c3)[nH]c(=O)c2c1C(F)(F)F]

A mixture of diethyl 1-[2-(3-chloro-4-methylphenyl)-2-oxoethyl]-4-(trifluoromethyl)-1H-pyrazole-3,5-dicarboxylate (Intermediate 219A, 1.20 g, 79% purity, 2.12 mmol) and Ammonium acetate (6.54 g, 84.9 mmol) in acetic acid (42 ml) was stirred at 110° C. overnight. After cooling to RT, the mixture was poured into water (600 ml) and the precipitate was collected by filtration and dried to afford the title compound (920 mg, quant., 93% purity) which was used in the next step without further purification.

LC/MS [Method 3]: Rₜ=2.11 min; MS (ESIpos): m/z=400 [M+H]⁺.

### Intermediate 221A

### 6-(3-Chloro-4-methylphenyl)-4-oxo-3-(trifluoromethyl)-4,5-dihydropyrazolo[1,5-a]pyrazine-2-carboxylic acid

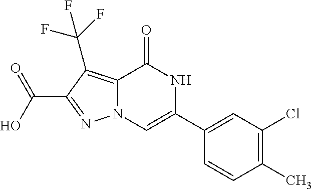 [SMILES: Cc1ccc(-c2cn3nc(C(=O)O)c(C(F)(F)F)c3c(=O)[nH]2)cc1Cl]

A mixture of ethyl 6-(3-chloro-4-methylphenyl)-4-oxo-3-(trifluoromethyl)-4,5-dihydropyrazolo-[1,5-a]pyrazine-2-carboxylate (Intermediate 220A, 920 mg, 93% purity, 2.14 mmol) and lithium hydroxide (513 mg, 21.4 mmol) in methanol (100 ml) and water (20 ml) was stirred at RT for 4 h. The methanol was removed under reduced pressure and the resulting aqueous phase was adjusted to pH 2 by addition of 1.0 M hydrochloric acid. The precipitate was collected by filtration and dried to afford the title compound (700 mg, 88% of theory).

LC/MS [Method 7]: Rₜ=0.81 min; MS (ESIneg): m/z=370 [M−H]⁻.

¹H-NMR (400 MHz, DMSO-d₆): δ [ppm]=14.00 (br s, 1H), 12.10 (s, 1H), 8.28 (s, 1H), 7.87 (d, 1H), 7.66 (dd, 1H), 7.49 (d, 1H), 2.39 (s, 3H).

### Intermediate 222A

### Diethyl 1-[2-(5-methylquinolin-3-yl)-2-oxoethyl]-1H-pyrazole-3,5-dicarboxylate

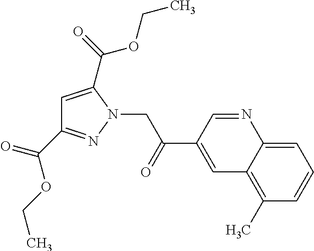 [SMILES: CCOC(=O)c1cc(C(=O)OCC)n(CC(=O)c2cnc3cccc(C)c3c2)n1]

To a solution of 2-bromo-1-(5-methylquinolin-3-yl)ethanone (Intermediate 74A, 5.81 g, 22.0 mmol) in acetone (300 ml) were added diethyl 1H-pyrazole-3,5-dicarboxylate (4.67 g, 22.0 mmol) and potassium carbonate (9.12 g, 66.00 mmol). The mixture was stirred at room temperature overnight. After filtering off the solids, the filtrate was concentrated under reduced pressure to provide the crude product. The residue was purified by flash chromatography on silica gel (eluent: petroleum ether-ethyl acetate 6:1) to give the title compound (7.23 g, 81% of theory, 97% purity).

LC/MS [Method 20]: Rₜ=1.04 min; MS (ESIpos): m/z=396 [M+H]⁺.

¹H-NMR (300 MHz, DMSO-d₆): δ [ppm] 9.38 (s, 1H), 9.17 (s, 1H), 8.00 (d, 1H), 7.84-7.98 (m, 1H), 7.60-7.63 (m, 1H), 7.40 (s, 1H), 2.81 (s, 3H), 6.52 (s, 2H), 4.33 (q, 2H), 4.23 (q, 2H), 1.30-1.35 (t, 3H), 1.20 (t, 3H).

### Intermediate 223A

### Ethyl 6-(5-methylquinolin-3-yl)-4-oxo-4,5-dihydropyrazolo[1,5-a]pyrazine-2-carboxylate

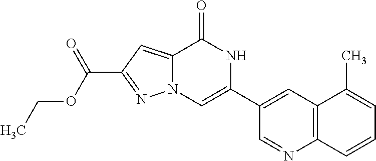 [SMILES: CCOC(=O)c1cc2c(=O)[nH]c(-c3cnc4cccc(C)c4c3)cn2n1]

A solution of diethyl 1-[2-(5-methylquinolin-3-yl)-2-oxoethyl]-1H-pyrazole-3,5-dicarbonlate (Intermediate 222A, 7.23 g, purity 97%, 17.7 mmol) and ammonium acetate (27.3 g, 355 mmol) in acetic acid (100 ml) was stirred overnight at 110° C. The solution was poured into ice-water, then filtered and the filter cake washed with water. Drying of the solids in air gave 6.15 g (quant.) of the title compound.

¹H-NMR (300 MHz, DMSO-d₆): δ [ppm] 12.15 (br s, 1H), 9.28 (s, 1H), 8.63 (s, 1H), 7.93-7.90 (m, 1H), 7.75-7.70 (m, 1H), 7.55-7.52 (m, 1H), 7.48 (s, 1H), 4.37 (q, 2H), 8.86 (s, 1H), 2.77 (s, 3H), 1.36(t, 3H).

### Intermediate 224A

### 6-(5-Methylquinolin-3-yl)-4-oxo-4,5-dihydropyrazolo[1,5-a]pyrazine-2-carboxylic acid hydrochloride

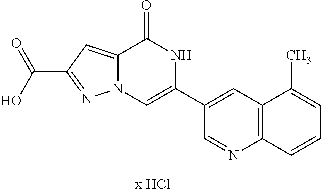 [SMILES: Cc1cccc2ncc(-c3cn4nc(C(=O)O)cc4c(=O)[nH]3)cc12]

To a solution of ethyl 6-(5-methylquinolin-3-yl)-4-oxo-4,5-dihydropyrazolo[1,5-a]pyrazine-2-carboxylate (Intermediate 223A, 6.15 g, 17.65 mmol) in ethanol (300 ml), aqueous sodium hydroxide (20 ml, 3 N) was added. After stirring for 4 hours at room temperature the mixture was extracted with ethyl acetate (100 ml). The pH value of aqueous phase was adjusted to 1 with diluted hydrochloric acid (3 M). The precipitate was filtered, washed with water and dried in air to give the title compound (6.30 g, 97% of theory, 97% purity).

LC/MS [Method 39]: Rₜ=0.85 min; MS (ESIpos): m/z=321 [M+H]⁺.

¹H-NMR (300 MHz, DMSO-d₆): δ [ppm] 12.17 (br s, 1H), 9.40 (s, 1H), 9.03 (s, 1H), 8.63 (s, 1H), 8.00 (d, 1H), 7.84-7.78 (m, 1H), 7.61 (d, 1H), 7.43 (s, 1H), 2.80 (s, 3H).

### Intermediate 225A

### [(2R)-2-(tert-Butoxycarbonylamino)-2-phenyl-ethyl]methanesulfonate

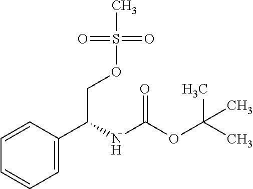 [SMILES: CC(C)(C)OC(=O)N[C@@H](COS(C)(=O)=O)c1ccccc1]

The title compound was prepared according to J. Med. Chem. 1994, 37, 1810 from N-boc-D-phenylglycinole (2.00 g). Yield: 2.31 g (90% of theory; pure by LC, contains some impurities by NMR). This material was not further purified.

¹H-NMR (400 MHz, DMSO-d₆): δ [ppm]=7.66 (d, 1H), 7.38-7.27 (m, 5H), 4.88 (m, 1H), 4.26 (m, 2H), 3.15 (s, 3H), 1.38 (s, 9H).

### Intermediate 226A

### tert-Butyl N-[(1R)-2-azido-1-phenyl-ethyl]carbamate

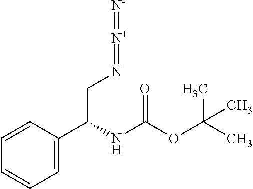 [SMILES: CC(C)(C)OC(=O)N[C@@H](CN=[N+]=[N-])c1ccccc1]

to Sodium azide (216 mg, 3.33 mmol) was added at RT to a solution of [(2R)-2-(tert-butoxycarbonylamino)-2-phenyl-ethyl] methanesulfonate (Intermediate 225A, 500 mg, 1.59 mmol) in DMF (10 ml). This mixture was stirred overnight at RT, then heated to 60° C. and stirred overnight again. The mixture was cooled to RT and diluted with water. The precipitate was washed with water and dried under reduced pressure at RT, yielding the title compound (310 mg, 73% of theory).

LC/MS [Method 3]: Rₜ=1.94 min; MS (ESIpos): m/z=163 (M+H-COOtBu)⁺.

¹H-NMR (400 MHz, DMSO-d₆): δ [ppm]=7.64 (d, 1H), 7.37-7.25 (m, 5H), 4.73 (m, 1H), 4.49 (m, 2H), 1.38 (s, 9H).

### Intermediate 227A

### (1R)-2-Azido-1-phenyl-ethanamine hydrochloride

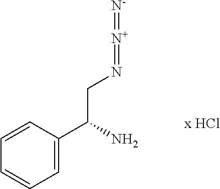 [SMILES: [N-]=[N+]=NC[C@H](N)c1ccccc1]

A solution of tert-butyl N-[(1R)-2-azido-1-phenyl-ethyl]carbamate (Intermediate 226A, 300 mg, 1.44 mmol) and methoxybenzene (0.62 ml; 5.72 mmol) in 1,4-dioxane (5 ml) was treated at RT with 4 N hydrogen chloride solution in dioxane (2.86 ml, 11.4 mmol). After stirring overnight, the solution was evaporated. The residue was taken up with ethanol and evaporated (three times). The solid was dried in high vacuum. This material (239 mg, quantitative yield) was used without further purification, some impurities detectable by NMR.

¹H-NMR (400 MHz, DMSO-d₆): δ [ppm]=8.8 (bs, 3H), 7.55 (m, 2H), 7.44 (m, 3H), 4.49 (m, 1H), 3.86 (m, 2H).

### Intermediate 228A

### (3R)-3-(1,3-Dioxoisoindolin-2-yl)-3-(4-fluorophenyl)propanoic acid

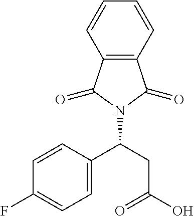 [SMILES: O=C(O)C[C@H](c1ccc(F)cc1)N1C(=O)c2ccccc2C1=O]

A mixture of phtalic anhydride (400 mg, 2.18 mmol) and (3R)-3-amino-3-(4-fluorophenyl)-propanoic acid (323 mg, 2.18 mmol) in DMF (2.8 ml) was heated to 120° for 110 min in a microwave oven. After cooling, the mixture was filtered and the solids were washed with water and dried at 40° in vacuo overnight to afford the title compound (600 mg, 83% of theory, 95% purity).

¹⁹F-NMR (376.6 MHz, DMSO-d₆): δ [ppm]=−114.7 (m).

### Intermediate 229A

### tert-Butyl N-[(2S)-2-(1,3-dioxoisoindolin-2-yl)-2-(4-fluorophenyl)ethyl]carbamate

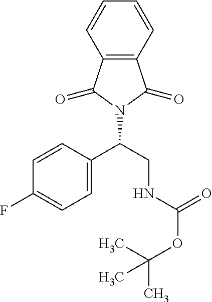 [SMILES: CC(C)(C)OC(=O)NC[C@H](c1ccc(F)cc1)N1C(=O)c2ccccc2C1=O]

In an oven-dried flask (3R)-3-(1,3-dioxoisoindolin-2-yl)-3-(4-fluorophenyl)propanoic acid (Intermediate 228A, 3.50 g, 11.2 mmol) in t-butanol (35 ml) was treated with diphenylphosporyl azide (4.61 g, 16.8 mmol) and trimethylamine (3.34 ml, 16.8 mmol) and heated for 90 min under argon to 80° C. The crude mixture was combined with a previous batch, prepared from 300 mg (3R)-3-(1,3-dioxoisoindolin-2-yl)-3-(4-fluorophenyl)propanoic acid. The combined crude reaction mixtures were evaporated and taken up with methyl t-butyl ether. After aqueous work-up and a final washing with brine the ethereal solution was filtered through a water-repellent filter and evaporated. The crude product was purified by silica chromatography (eluent: gradient of hexane/ethylacetat) to afford the title compound (2.06 g, 43% of theory, 90% purity).

¹⁹F-NMR (376.6 MHz, DMSO-d₆): δ [ppm]=−115.0 (m).

### Intermediate 230A

### 2-[(1S)-2-Amino-1-(4-fluorophenyl)ethyl]isoindoline-1,3-dione; 2,2,2-trifluoroacetic acid salt

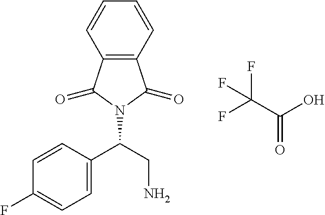

tert-Butyl N-[(2S)-2-(1,3-dioxoisoindolin-2-yl)-2-(4-fluorophenyl)ethyl]carbamate (Intermediate 229A, 2.06 g, 5.36 mmol) was stirred for 2 h at RT in 20 ml of a dichloromethane/trifluoroacetic acid mixture (1:1; v/v). The mixture was evaporated, the residue treated several times with dichloromethane and taken up with toluene and evaporated to dryness. This yielded, after drying in vacuo, the title compound (2.18 g, 97% of theory, 95% purity) which was used without further purification.

¹⁹F-NMR (376.6 MHz, DMSO-d₆): δ [ppm]=−73.9 (s), −113.9 (m).

### Intermediate 231A

### 2-[(1S)-1-(4-Fluorophenyl)-2-morpholino-ethyl]isoindoline-1,3-dione

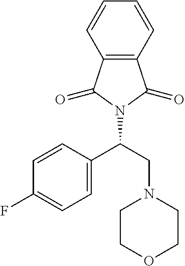 [SMILES: O=C1c2ccccc2C(=O)N1[C@H](CN1CCOCC1)c1ccc(F)cc1]

2-[(1S)-2-Amino-1-(4-fluorophenyl)ethyl]isoindoline-1,3-dione 2,2,2-trifluoroacetic acid salt (Intermediate 230A, 300 mg, 1.06 mmol) and potassium carbonate (437.5 mg, 3.17 mmol) in acetonitrile (4 ml) were treated with 1-bromo-2-(2-bromoethoxy)ethane. The mixture was heated for 90 min at 110° C. in the microwave oven. This mixture was combined with 3 identical batches for work-up. After evaporation to approx. 1/5 of the initial volume, the mixture was diluted with water and dichloromethane. After aqueous work-up and a final washing with brine, the organic phase was filtered through a water repellent filter and evaporated to afford the title compound (1.66 g). The crude product was used in the next step without further purification.

### Intermediate 232A

### (1S)-1-(4-Fluorophenyl)-2-morpholino-ethanamine

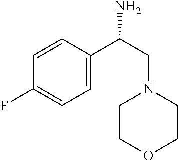 [SMILES: N[C@H](CN1CCOCC1)c1ccc(F)cc1]

In a microwave vial, a solution of 2-[(1S)-1-(4-fluorophenyl)-2-morpholino-ethyl]isoindoline-1,3-dione (Intermediate 231A, 320 mg, 0.90 mmol) in ethanol (3.3 ml) was treated with hydrazine hydrate (0.22 ml, 4.52 mmol) and heated to 100° C. in the microwave oven. The mixture was diluted with ethanol and filtered. The solids were washed with ethanol. The filtrate was combined with the filtrates from identical runs (in total: 1.60 g starting material employed) and concentrated in vacuo. This was diluted with dichloromethane, stirred for 5 min and filtered. The filtrate was concentrated to afford the title compound (680 mg) which was used in the next steps without further purification.

¹H-NMR (400 MHz, DMSO-d₆): δ [ppm]=7.43 (dd, 2H), 7.10 (m, 2H), 4.08 (dd, 1H), 3.6-3.35 (m, 7H), 2.75-2-73 (m, 1H), 2.34-2.21 (m, 3H) and signals of impurities between 2.5 and 4 ppm.

### Intermediate 233A

### tert-Butyl 3-[methoxy(methyl)carbamoyl]azetidine-1-carboxylate

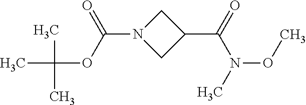 [SMILES: CON(C)C(=O)C1CN(C(=O)OC(C)(C)C)C1]

Carbonyldiimidazole (24.4 g, 150 mmol) was added in portions to a solution of 1-tert-butoxycarbonylazetidine-3-carboxylic acid (23.2 g, 115 mmol) in THF (250 ml) and the mixture was stirred at room temperature for 1.5 h. A suspension of N,O-dimethylhydroxylamine hydrochloride (15.0 g, 154 mmol) in a mixture of acetonitrile (300 ml) and triethylamine (22.0 ml, 162 mmol) was added and the reaction was stirred at room temperature for 24 h. The solvents were evaporated and the residue was partitioned between water (300 ml) and ethyl acetate (800 ml). The organic layer was separated, washed with a 5% aqueous citric acid solution (400 ml), water (300 ml) and brine (300 ml), dried over anhydrous magnesium sulfate, and concentrated in vacuo to afford the title compound (28.2 g, quant).

### Intermediate 234A

### tert-Butyl 3-(4-fluorobenzoyl)azetidine-1-carboxylate

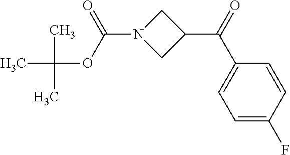 [SMILES: CC(C)(C)OC(=O)N1CC(C(=O)c2ccc(F)cc2)C1]

n-Butyl lithium (76.8 ml, 123 mmol, 1.6M solution of in hexane) was added dropwise to a solution of 1-bromo-4-fluorobenzene (20.0 g, 114 mmol) in dry THF (200 ml) at −78° C. under nitrogen and the reaction was stirred at the same temperature for 1 h. Then, a solution of tent-butyl 3-[methoxy(methyl)carbamoyl]azetidine-1-carboxylate (Intermediate 233A, 20.0 g. 81.9 mmol) in THF (75 ml) was added. The reaction mixture was stirred for 1 h at −78° C. and then quenched with ice. The product was extracted with ethyl acetate (2×100 ml). The combined organic layers were dried over sodium sulfate and evaporated under reduced pressure. The residue was purified by silica gel column chromatography (petroleum ether/ethyl acetate 80:20) to afford the title compound (21.9 g, 96% of theory).

### Intermediate 235A

### tert-butyl 3-[(Z)-C-(4-fluorophenyl)-N-hydroxy-carbonimidoyl]azetidine-1-carboxylate

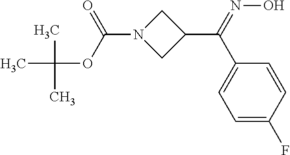 [SMILES: CC(C)(C)OC(=O)N1CC(/C(=N/O)c2ccc(F)cc2)C1]

To a solution of tert-butyl 3-(4-fluorobenzoyl)azetidine-1-carboxylate (Intermediate 234A, 14.0 g, 50.1 mmol) in a mixture of methanol (120 ml) and water (20 ml), sodium acetate (10.2 g, 125 mmol) was added followed by hydroxyl amine hydrochloride (6.90 g, 100 mmol) at room temperature. The reaction mixture was stirred at room temperature for 12 h and then concentrated under reduced pressure. The residue was dissolved in ethyl acetate (200 ml), washed with 10% aqueous solution of sodium bicarbonate (100 ml), water (100 ml), and brine (100 ml), dried over sodium sulfate, and evaporated under reduced pressure. The crude material was purified by column chromatography (petroleum ether/ethyl acetate 70:30) to afford the title compound (13.0 g, 88% of theory).

### Intermediate 236A

### tert-Butyl 3-[amino-(4-fluorophenyl)methyl]azetidine-1-carboxylate (Racemate)

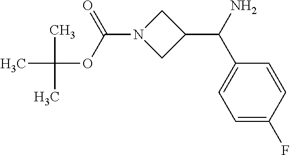 [SMILES: CC(C)(C)OC(=O)N1CC(C(N)c2ccc(F)cc2)C1]

tert-Butyl 3-[(Z)-C-(4-fluorophenyl)-N-hydroxy-carbonimidoyl]azetidine-1-carboxylate (Intermediate 235A, 12.0 g, 40.7 mmol) was stirred in degassed methanol (300 ml) under a pressure of hydrogen (10 atm.) in the presence of Pd/C (3.00 g, 10% wt.) for 12 hours at room temperature. The suspension was filtered and the resultant solution was concentrated under reduced pressure. The residue was taken up in 1% aqueous solution of citric acid (50 ml) and washed with ethyl acetate (2×100 ml). The separated aqueous layer was basified with 10% aqueous solution of sodium bicarbonate (40 ml) and extracted with dichloromethane (2×100 ml). The dichloromethane layer was washed with brine, dried over sodium sulfate and evaporated under reduced pressure to afford the title compound (8.60 g, 74% of theroy).

¹H-NMR (400 MHz, DMSO-d₆): δ [ppm]=7.42-7.35 (m, 2H), 7.14-7.07 (m, 2H), 3.89-3.72 (m, 3H), 3.66-3.53 (m, 1H), 3.52-3.35 (m, 1H), 2.71-2.58 (m, 1H), 2.14-1.93 (m, 2H), 1.35 (s, 9H).

### Intermediate 237A

Diethyl 1-[2-(4-chloro-3-methylphenyl)-2-oxoethyl]-4-(trifluoromethyl)-1H-pyrazole-3,5-dicarboxylate

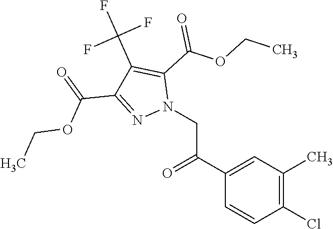 [SMILES: CCOC(=O)c1nn(CC(=O)c2ccc(Cl)c(C)c2)c(C(=O)OCC)c1C(F)(F)F]

To a solution of diethyl 4-(trifluoromethyl)-1H-pyrazole-3,5-dicarboxylate (Intermediate 32A, 500 mg, 1.78 mmol) in acetone (16 ml) were added 2-bromo-1-(4-chloro-3-methylphenyl)ethanone (546 mg, 2.14 mmol) and potassium carbonate (616 mg, 4.46 mmol). The mixture was stirred at RT for 1 h. The reaction mixture was then filtered, the filter cake was washed with acetone, and the combined filtrates were evaporated to dryness. The crude product was purified by flash chromatography on silica gel (cyclohexan/ethylacetat gradient). Yield: 640 mg (80% of theory).

LC/MS [Method 3]: Rₜ=2.40 min; MS (ESIpos): m/z=447 [M+H]⁺.

¹H-NMR (500 MHz, DMSO-d₆): δ [ppm]=8.04 (s, 1H), 7.88 (dd, 1H), 7.67 (d, 1H), 6.26 (s, 2H), 4.36 (q, 2H), 4.26 (q, 2H), 2.44 (s, 3H), 1.30 (t, 3H), 1.14 (t, 3H).

### Intermediate 238A

### Ethyl 6-(4-chloro-3-methylphenyl)-4-oxo-3-(trifluoromethyl)-4,5-dihydropyrazolo[1,5-a]pyrazine-2-carboxylate

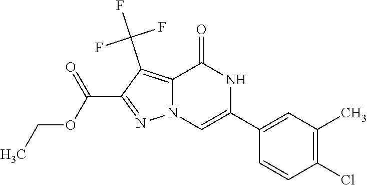 [SMILES: CCOC(=O)c1nn2cc(-c3ccc(Cl)c(C)c3)[nH]c(=O)c2c1C(F)(F)F]

A mixture of diethyl 1-[2-(4-chloro-3-methylphenyl)-2-oxoethyl]-4-(trifluoromethyl)-1H-pyrazole-3,5-dicarboxylate (Intermediate 237A, 640 mg, 1.43 mmol) and ammonium acetate (1.10 g, 14.3 mmol) in acetic acid (6 ml) was heated to 110° C. for 2 days. After cooling to RT, the mixture was poured into water (100 ml), and the precipitate was collected by filtration, washed with water and dried to afford the title compound. Yield: 525 mg (92% of theory).

LC/MS [Method 7]: Rₜ=1.11 min; MS (ESIpos): m/z=400 [M+H]⁺.

¹H-NMR (500 MHz, DMSO-d₆): δ [ppm]=12.12 (s, 1H), 8.29 (s, 1H), 7.80 (s, 1H), 7.62 (dd, 1H), 7.56 (d, 1H), 4.40 (q, 2H), 2.40 (s, 3H), 1.32 (t, 3H).

### Intermediate 239A

### 6-(4-Chloro-3-methylphenyl)-4-oxo-3-(trifluoromethyl)-4,5-dihydropyrazolo[1,5-a]pyrazine-2-carboxylic acid

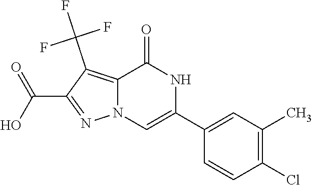 [SMILES: Cc1cc(-c2cn3nc(C(=O)O)c(C(F)(F)F)c3c(=O)[nH]2)ccc1Cl]

A mixture of ethyl 6-(3,4-dimethylphenyl)-4-oxo-3-(trifluoromethyl)-4,5-dihydropyrazolo[1,5-a]-pyrazine-2-carboxylate (Intermediate 238A, 525 mg, 1.13 mmol) and lithium hydroxide (157 mg, 6.57 mmol) in methanol (25 ml) and water (5.0 ml) was stirred at RT for 3 h. The methanol was then removed under reduced pressure. The remaining aqueous phase was diluted with water brought to pH 2 by addition of 1.0 M hydrochloric acid. The precipitate was collected by filtration and dried to afford the title compound. Yield 490 mg (100% of theory).

LC/MS [Method 3]: Rₜ=1.48 min; MS (ESIpos): m/z=372 [M+H]⁺.

¹H-NMR (500 MHz, DMSO-d₆): δ [ppm]=14.01 (br s, 1H), 12.08 (s, 1H), 8.24 (s, 1H), 7.80 (s, 1H), 7.62 (dd, 1H), 7.55 (d, 1H), 2.40 (s, 3H).

### Intermediate 240A

### Diethyl 4-cyclopropyl-1-{2-[3-fluoro-4-methylphenyl]-2-oxoethyl}-1H-pyrazole-3,5-dicarboxylate

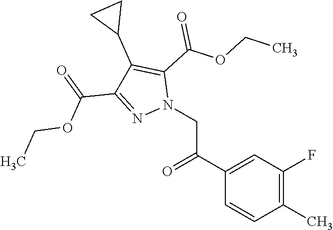 [SMILES: CCOC(=O)c1nn(CC(=O)c2ccc(C)c(F)c2)c(C(=O)OCC)c1C1CC1]

A mixture of diethyl 4-cyclopropyl-1H-pyrazole-3,5-dicarboxylate (Intermediate 187A, 1500 mg, 5.95 mmol), 2-bromo-1-[3-fluoro-4-methylphenyl]ethan-1-one (1.65 g, 7.14 mmol) and potassium carbonate (2.05 g, 14.8 mmol) in acetone (52 ml) was stirred at RT for 1 hour. The insoluble material was filtered off, washed with acetone and the filtrate was concentrated under reduced pressure to give the crude title compound which was directly used in the next step without further purification. Yield: 2.6 g (97% of theory, 90% purity).

LC/MS [Method 3]: Rₜ=2.25 min; MS (ESIpos): m/z=403 [M+H]⁺.

### Intermediate 241A

### Ethyl 3-cyclopropyl-6-[3-fluoro-4-methylphenyl]-4-oxo-4,5-dihydropyrazolo[1,5-a]pyrazine-2-carboxylate

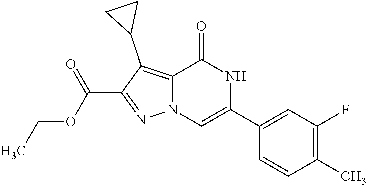 [SMILES: CCOC(=O)c1nn2cc(-c3ccc(C)c(F)c3)[nH]c(=O)c2c1C1CC1]

A mixture of diethyl 4-cyclopropyl-1-{2-[3-fluoro-4-methylphenyl]-2-oxoethyl}-1H-pyrazole-3,5-dicarboxylate (Intermediate 240A, 1.72 g, 90% purity, 3.9 mmol) and ammonium acetate (12.2 g, 158 mmol) in acetic acid (60 ml) was heated to 110° C. for 3 days. After cooling to RT, the reaction mixture was poured into water (300 ml). The precipitate was collected by filtration, washed with water (50 ml) and dried to afford the title compound. Yield: 1.20 g (85% of theory).

LC/MS [Method 3]: Rₜ=1.99 min; MS (ESIpos): m/z=356 [M+H]⁺.

### Intermediate 242A

### 3-Cyclopropyl-6-[3-fluoro-4-methylphenyl]-4-oxo-4,5-dihydropyrazolo[1,5-a]pyrazine-2-carboxylic acid

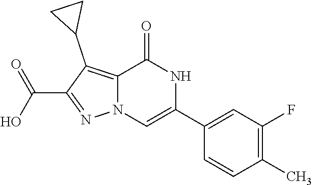 [SMILES: Cc1ccc(-c2cn3nc(C(=O)O)c(C4CC4)c3c(=O)[nH]2)cc1F]

A mixture of ethyl 3-cyclopropyl-6-[3-fluoro-4-methylphenyl]-4-oxo-4,5-dihydropyrazolo[1,5-a]pyrazine-2-carboxylate (Intermediate 241A, 1.20 g, 3.38 mmol) and 1 N aqueous lithium hydroxide solution (16.9 ml, 16.9 mmol) in 25.5 ml of a mixture of tetrahydrofuran and methanol (5:1 v/v was stirred at RT for 1 h. The solvents were distilled off under reduced pressure, and the residue was diluted with water and acidified by addition of 1.0 M hydrochloric acid. The precipitate was collected by filtration and dried to afford the title compound. Yield: 1.10 g (97% of theory).

LC/MS [Method 3]: Rₜ=1.47 min; MS (ESIpos): m/z=328 [M+H]⁺.

¹H-NMR (500 MHz, DMSO-d₆): δ [ppm]=13.12 (br. s, 1H), 11.45 (s, 1H), 8.08 (s, 1H), 7.59 (dd, 1H), 7.52 (dd, 1H), 7.39 (t, 1H), 2.76-2.70 (m, 1H), 1.27-1.23 (m, 2H), 0.95-0.90 (m, 2H).

### Intermediate 243A

### Diethyl 4-methyl-1-{2-[3-fluoro-4-methylphenyl]-2-oxoethyl}-1H-pyrazole-3,5-dicarboxylate

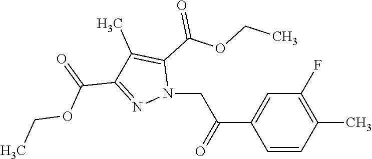 [SMILES: CCOC(=O)c1nn(CC(=O)c2ccc(C)c(F)c2)c(C(=O)OCC)c1C]

A mixture of diethyl 4-methyl-1H-pyrazole-3,5-dicarboxylate (Intermediate 54A, 300 mg, 1.33 mmol), 2-bromo-1-[3-fluoro-4-methylphenyl]ethan-1-one (368 mg, 1.59 mmol) and potassium carbonate (458 mg, 3.31 mmol) in acetone (12 ml) was stirred at RT overnight. The insoluble material was filtered off, washed with acetone and the filtrate was concentrated under reduced pressure to give the crude title compound which was directly used in the next step without further purification. Yield: 509 mg (93% of theory, 91% purity).

LC/MS [Method 3]: Rₜ=2.16 min; MS (ESIpos): m/z=377 [M+H]⁺.

### Intermediate 244A

### Ethyl 3-methyl-6-[3-fluoro-4-methylphenyl]-4-oxo-4,5-dihydropyrazolo[1,5-a]pyrazine-2-carboxylate

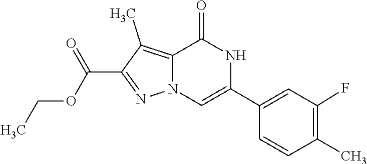 [SMILES: CCOC(=O)c1nn2cc(-c3ccc(C)c(F)c3)[nH]c(=O)c2c1C]

A mixture of diethyl 4-methyl-1-{2-[3-fluoro-4-methylphenyl]-2-oxoethyl}-1H-pyrazole-3,5-dicarboxylate (Intermediate 243A, 509 mg, 91% purity, 1.24 mmol) and ammonium acetate (3.81 g, 49.4 mmol) in acetic acid (25 ml) was heated to 110° C. for 3 days. After cooling to RT, the reaction mixture was poured into water (100 ml). The precipitate was collected by filtration, washed with water (30 ml) and dried to afford the title compound, which was directly used in the next step without further purification. Yield: 364 mg (80% of theory, 89% purity).

LC/MS [Method 3]: Rₜ=1.82 min; MS (ESIpos): m/z=330 [M+H]⁺.

### Intermediate 245A

### 3-Methyl-6-[3-fluoro-4-methylphenyl]-4-oxo-4,5-dihydropyrazolo[1,5-a]pyrazine-2-carboxylic acid

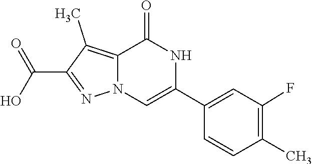 [SMILES: Cc1ccc(-c2cn3nc(C(=O)O)c(C)c3c(=O)[nH]2)cc1F]

A mixture of ethyl 3-methyl-6-[3-fluoro-4-methylphenyl]-4-oxo-4,5-dihydropyrazolo[1,5-a]pyrazine-2-carboxylate (Intermediate 244A, 364 mg, 1.11 mmol) and 1 N aqueous lithium hydroxide solution (5.53 ml, 5.53 mmol) in 8.40 ml of a mixture of tetrahydrofuran and methanol (5:1 v/v %) was stirred at RT for 3 h. The solvents were distilled off under reduced pressure, and the residue was diluted with water and acidified by addition of 1.0 M hydrochloric acid. The precipitate was collected by filtration, washed with water and dried to afford the title compound. Yield: 330 mg (99% of theory).

LC/MS [Method 3]: Rₜ=1.35 min; MS (ESIpos): m/z=302 [M+H]⁺.

¹H-NMR (500 MHz, DMSO-d₆): δ [ppm]=13.12 (br. s, 1H), 11.50 (s, 1H), 8.09 (s, 1H), 7.59 (dd, 1H), 7.52 (dd, 1H), 7.39 (t, 1H), 2.63 (s, 3H), 2.28 (s, 3H).

### Intermediate 246A

### 2-Bromo-1-(2,3-difluoro-4-methylphenyl)ethanone

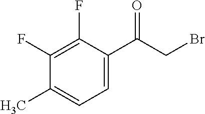

A solution of trimethylphenylammonium tribromide (2.21 g, 5.88 mmol) in 9.0 ml of THF was added in portions to a solution of 1-(2,3-difluoro-4-methylphenyl)ethanone (1.00 g, 5.88 mmol) in THF (9.0 ml) at 50° C. The mixture was subsequently stirred at 50° C. for 1 hour. After completion of the reaction, the mixture was filtered, and the filtrate was concentrated to afford the crude product which was used in the next step without further purification. Yield: 2.83 g quant., 78% purity). GC/MS [Method 35]: Rₜ=4.46 min; MS (ESIpos): m/z=248 [M+H]⁺.

### Intermediate 247A

### Diethyl 4-methyl-1-{2-[2,3-difluoro-4-methylphenyl]-2-oxoethyl}-1H-pyrazole-3,5-dicarboxylate

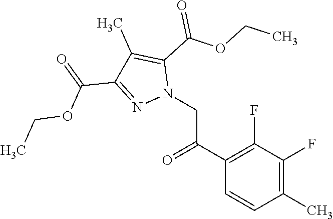 [SMILES: CCOC(=O)c1nn(CC(=O)c2ccc(C)c(F)c2F)c(C(=O)OCC)c1C]

A mixture of diethyl 4-methyl-1H-pyrazole-3,5-dicarboxylate (Intermediate 54A, 469 mg, 2.08 mmol), 2-bromo-1-(2,3-difluoro-4-methylphenyl)ethanone (Intermediate 246A, 795 mg, 78% purity, 2.49 mmol) and potassium carbonate (717 mg, 5.19 mmol) in acetone (18 ml) was stirred at RT overnight. The insoluble material was filtered off, washed with acetone and the filtrate was concentrated under reduced pressure to give the crude title compound which was directly used in the next step without further purification. Yield: 850 mg (92% of theory, 88% purity).

LC/MS [Method 3]: Rₜ=2.23 min; MS (ESIpos): m/z=395 [M+H]⁺.

### Intermediate 248A

### Ethyl 3-methyl-6-[2,3-difluoro-4-methylphenyl]-4-oxo-4,5-dihydropyrazolo[1,5-a]pyrazine-2-carboxylate

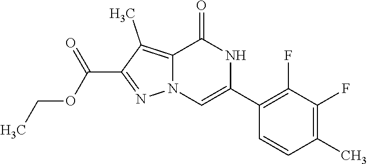 [SMILES: CCOC(=O)c1nn2cc(-c3ccc(C)c(F)c3F)[nH]c(=O)c2c1C]

A mixture of diethyl 4-methyl-1-{2-[2,3-difluoro-4-methylphenyl]-2-oxoethyl}-1H-pyrazole-3,5-dicarboxylate (Intermediate 247A, 850 mg, 88% purity, 1.90 mmol) and ammonium acetate (5.88 g, 76.2 mmol) in acetic acid (38 ml) was heated to 110° C. for 3 days. After cooling to RT, the reaction mixture was poured into water (150 ml). The precipitate was collected by filtration, washed with water (30 ml) and dried to afford the title compound, which was directly used in the next step without further purification. Yield: 615 mg (88% of theory, 95% purity).

LC/MS [Method 3]: Rₜ=1.86 min; MS (ESIpos): m/z=348 [M+H]⁺.

### Intermediate 249A

### 3-Methyl-6-[2,3-difluoro-4-methylphenyl]-4-oxo-4,5-dihydropyrazolo[1,5-a]pyrazine-2-carboxylic acid

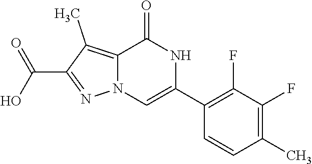 [SMILES: Cc1ccc(-c2cn3nc(C(=O)O)c(C)c3c(=O)[nH]2)c(F)c1F]

A mixture of ethyl 3-methyl-6-[2,3-difluoro-4-methylphenyl]-4-oxo-4,5-dihydropyrazolo[1,5-a]pyrazine-2-carboxylate (Intermediate 248A, 139 mg, 0.42 mmol) and 1 N aqueous lithium hydroxide solution (2.08 ml, 2.08 mmol) in 3.16 ml of a mixture of tetrahydrofuran and methanol (5:1 v/v was stirred at RT for 1 h. The solvents were distilled off under reduced pressure, and the residue was diluted with water and acidified by addition of 1.0 M hydrochloric acid. The precipitate was collected by filtration, washed with water and dried to afford the title compound. Yield: 111 mg (87% of theory).

LC/MS [Method 3]: Rₜ=1.32 min; MS (ESIpos): m/z=320 [M+H]⁺.

¹H-NMR (500 MHz, DMSO-d₆): δ [ppm]=11.59 (br s, 1H), 7.88 (s, 1H), 7.34 (t, 1H), 7.24 (t, 1H), 2.63 (s, 3H), 2.35 (s, 3H).

### Intermediate 250A

### 1-(2-Fluoro-3,4-dimethylphenyl)ethanone

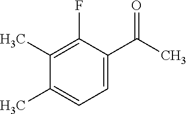

To a 1.4 M solution of sec-butyllithium in cyclohexane (12.1 ml, 16.9 mmol) was slowly added at −70° C. a solution of 1-fluoro-2,3-dimethylbenzene (2.0 g, 16.1 mmol) in THF (45 ml) and stirred for 1 hour at −70° C. Then, N-methoxy-N-methylacetamide was added and stirred for 20 minutes at −70° C. After that, the reaction mixture was carefully quenched with water (10 ml) and extracted three times with ethyl acetate. The combined organic phases were dried over sodium sulfate, filtered and the solvents were removed under reduced pressure. The crude product was purified by flash chromatography (cyclohexane/ethylacetate gradient). Yield: 909 mg (34% of theory, assumed purity of 70%).

GC/MS [Method 35]: Rₜ=3.70 min; MS (EIpos): m/z=166 [M]⁺.

### Intermediate 251A

### 2-Bromo-1-(2-fluoro-3,4-dimethylphenyl)ethanone

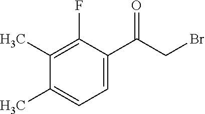

Trimethylphenylammonium tribromide (452 mg, 1.20 mmol) was added in portions to a solution of 1-(2-fluoro-3,4-dimethylphenyl)ethanone (Intermediate 250A, 200 mg, 1.20 mmol) in THF (2.0 ml) and stirred for 1 hour at RT. After completion of the reaction, the mixture was filtered, and the filtrate was concentrated to afford the crude product which was used in the next step without further purification. Yield: 434 mg quant., assumed purity of 70%).

LC/MS [Method 3]: Rₜ=1.98 min; MS (ESIpos): m/z=245 [M+H]⁺.

### Intermediate 252A

### Diethyl 4-methyl-1-{2-fluoro-3,4-dimethylphenyl]-2-oxoethyl}-1H-pyrazole-3,5-dicarboxylate

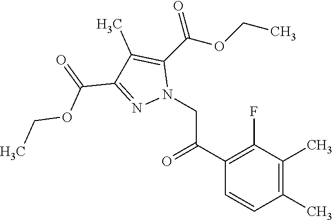 [SMILES: CCOC(=O)c1nn(CC(=O)c2ccc(C)c(C)c2F)c(C(=O)OCC)c1C]

A mixture of diethyl 4-methyl-1H-pyrazole-3,5-dicarboxylate (Intermediate 54A, 498 mg, 2.08 mmol), 2-bromo-1-(2-fluoro-3,4-dimethylphenyl)ethanone (Intermediate 251A, 924 mg, 70% purity, 2.64 mmol) and potassium carbonate (760 mg, 5.50 mmol) in acetone (19 ml) was stirred at RT for 1 hour. The insoluble material was filtered off, washed with Aceton and the filtrate was concentrated under reduced pressure to give the crude title compound which was directly used in the next step without further purification. Yield: 1.13 g (104% of theory, 79% purity).

LC/MS [Method 3]: Rₜ=2.33 min; MS (ESIpos): m/z=391 [M+H]⁺.

### Intermediate 253A

### Ethyl 3-methyl-6-[2-fluoro-3,4-dimethylphenyl]-4-oxo-4,5-dihydropyrazolo[1,5-a]pyrazine-2-carboxylate

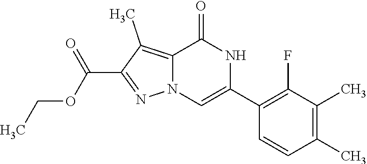 [SMILES: CCOC(=O)c1nn2cc(-c3ccc(C)c(C)c3F)[nH]c(=O)c2c1C]

A mixture of diethyl 4-methyl-1-{2-[2-fluoro-3,4-dimethylphenyl]-2-oxoethyl}-1H-pyrazole-3,5-dicarboxylate (Intermediate 252A, 1.13 g, 2.90 mmol) and ammonium acetate (4.47 g, 58.0 mmol) in acetic acid (16 ml) was heated to 110° C. for 3 days. After cooling to RT, the reaction mixture was poured into water (100 ml). The precipitate was collected by filtration, and dried to afford the title compound, which was directly used in the next step without further purification.

Yield: 643 mg (41% of theory, 63% purity).

LC/MS [Method 7]: Rₜ=1.02 min; MS (ESIpos): m/z=344 [M+H]⁺.

### Intermediate 254A

### 3-Methyl-6-[2-fluoro-3,4-dimethylphenyl]-4-oxo-4,5-dihydropyrazolo[1,5-a]pyrazine-2-carboxylic acid

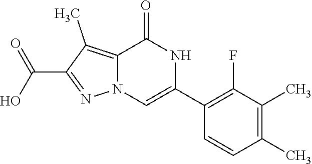 [SMILES: Cc1ccc(-c2cn3nc(C(=O)O)c(C)c3c(=O)[nH]2)c(F)c1C]

A mixture of ethyl 3-methyl-6-[2-fluoro-3,4-dimethylphenyl]-4-oxo-4,5-dihydropyrazolo[1,5-a]pyrazine-2-carboxylate (Intermediate 253A, 643 mg, 1.87 mmol) and 1 N aqueous lithium hydroxide solution (9.36 ml, 9.36 mmol) in 14.2 ml of a mixture of tetrahydrofuran and methanol (5:1 v/v %) was stirred at RT for 1 h. The solvents were distilled off under reduced pressure, and the residue was diluted with water and acidified by addition of 1.0 M hydrochloric acid. The precipitate was collected by filtration, washed with water and dried to afford the title compound. Yield: 572 mg (92% of theory).

LC/MS [Method 3]: Rₜ=1.43 min; MS (ESIpos): m/z=316 [M+H]⁺.

¹H-NMR (500 MHz, DMSO-d₆): δ [ppm]=13.07 (br s, 1H), 11.48 (s, 1H), 7.76 (s, 1H), 7.31 (t, 1H), 7.12 (d, 1H), 2.63 (s, 3H). 2.32 (s, 3H), 2.20 (s, 3H).

### Intermediate 255A

### 2-Bromo-1-(4-chloro-3-fluorophenyl)ethan-1-one

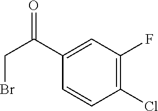 [SMILES: O=C(CBr)c1ccc(Cl)c(F)c1]

A mixture of 1-(4-chloro-3-fluorophenyl)ethan-1-one (3.00 g, 17.4 mmol) and phenyltrimethylammonium tribromide (7.19 g, 19.1 mmol) in dichloromethane (150 ml) was stirred overnight at RT. The insoluble materials were filtered off and the filter cake was washed with ethyl acetate. The combined filtrates were concentrated under reduced pressure and the residue was dried to afford the title compound (4.3 g, 75% of theory, 76% purity) which was used for the next step without further purification.

¹H-NMR (300 MHz, DMSO-d₆): δ [ppm]=8.02 (dd, 1H), 7.88-7.80 (m, 2H), 4.97 (s, 2H), 2.75-2.54 (m, 1H) ppm.

### Intermediate 256A

### Diethyl 1-[2-(4-chloro-3-fluoro-phenyl)-2-oxo-ethyl]-4-cyclopropyl-pyrazole-3,5-dicarboxylate

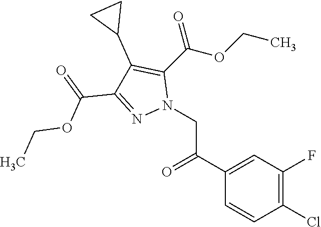 [SMILES: CCOC(=O)c1nn(CC(=O)c2ccc(Cl)c(F)c2)c(C(=O)OCC)c1C1CC1]

To a solution of 2-bromo-1-(4-chloro-3-fluorophenyl)ethan-1-one (Intermediate 255A, 4.84 g (75% purity, 14.4 mmol) in ethanol (150 ml) were added diethyl 4-cyclopropyl-1H-pyrazole-3,5-dicarboxylate (Intermediate 187A, 2.84 g, 95% purity, 10. 7 mmol) and potassium carbonate (4.40 g, 36.1 mmol). The mixture was stirred for 2 h at RT. The insoluble material was filtered off and the filtrate was concentrated under reduced pressure. The resulting crude product was suspended in ethyl acetate/petroleum ether (200 ml) (1:20) and stirred for 30 minutes. The formed precipitate was collected by filtrated to give the tile compound (3.88 g, 76% of theory 89% purity) of the title compound.

1H-NMR (300 MHz, DMSO-d₆): δ [ppm]=8.08 (dd, 1H), 7.95-7.84 (m, 2H), 6.16 (s, 2H), 4.30 (q, 2H), 4.20 (q, 2H), 2.13-2.01 (m, 1H), 1.31 (t, 3H), 1.16 (t, 3H), 0.99-0.87 (m, 2H), 0.73-0.63 (m, 2H).

### Intermediate 257A

### Ethyl 6-(4-chloro-3-fluorophenyl)-3-cyclopropyl-4-oxo-4,5-dihydropyrazolo[1,5-a]pyrazine-2-carboxylate

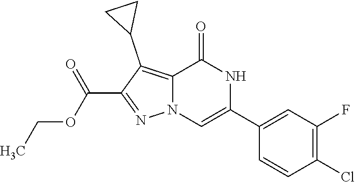 [SMILES: CCOC(=O)c1nn2cc(-c3ccc(Cl)c(F)c3)[nH]c(=O)c2c1C1CC1]

To a solution of diethyl 1-[2-(4-chloro-3-fluoro-phenyl)-2-oxo-ethyl]-4-cyclopropyl-pyrazole-3,5-dicarboxylate (Intermediate 256A, 3.88 g, 8.17 mmol, 89% purity) in acetic acid (150 ml) was added ammonium acetate (19.23 g, 24.50 mmol) and the resulting mixture was heated to 110° C. overnight. After cooling to room temperature, the reaction mixture was poured into ice-water. The precipitate was collected by filtration, washed with water and dried in vacuo to give the title compound (2.94 g, 91% of theory, 95% purity).

¹H-NMR (400 MHz, DMSO-d₆): δ [ppm]=11.94 (br s, 1H), 8.23 (s, 1H), 7.86 (dd, 1H), 7.74-7.68 (m, 1H), 7.68-7.62 (m, 1H), 4.34 (q, 2H), 2.69-2.59 (m, 1H), 1.33 (t, 3H), 1.27-1.17 (m, 2H), 1.01-0.91 (m, 2H).

### Intermediate 258A

### 6-(4-Chloro-3-fluorophenyl)-3-cyclopropyl-4-oxo-4,5-dihydropyrazolo[1,5-a]pyrazine-2-carboxylic acid

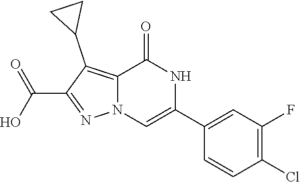 [SMILES: O=C(O)c1nn2cc(-c3ccc(Cl)c(F)c3)[nH]c(=O)c2c1C1CC1]

Sodium hydroxide (4.63 g,146.12 mmol) was added to a solution of ethyl 6-(4-chloro-3-fluoro-phenyl)-3-cyclopropyl-4-oxo-4,5-dihydropyrazolo[1,5-a]pyrazine-2-carboxylate (Intermediate 257A, 2.89 g, 95% purity, 7.31 mmol) in) in ethanol (50 ml) and water (33 ml). After stirring at room temperature for 2 hours, the reaction mixture was concentrated under reduced pressure. The residue was diluted with water and the pH value of the mixture was adjusted to 3-4 with hydrochloric acid. The precipitate was collected by filtration, washed with water and dried in air to give 2.28 g (89% of theory, 99% purity) of the title compound.

¹H-NMR (300 MHz, DMSO-d₆): δ [ppm]=13.12 (br s, 1H), 11.51 (s, 1H), 8.16 (s, 1H), 7.85 (dd, 1H), 7.76-7.55 (m, 2H), 2.78-2.63 (m, 1H), 1.30-1.14 (m, 2H), 1.00-0.82 (m, 2H).

### Intermediate 259A

### Diethyl 4-cyclopropyl-1-[2-(3,4-dichlorophenyl)-2-oxoethyl]-1H-pyrazole-3,5-dicarboxylate

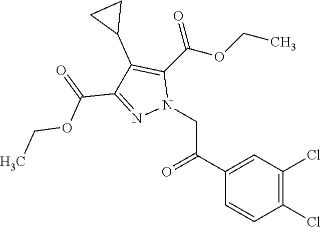 [SMILES: CCOC(=O)c1nn(CC(=O)c2ccc(Cl)c(Cl)c2)c(C(=O)OCC)c1C1CC1]

To a solution of diethyl 4-cyclopropyl-1H-pyrazole-3,5-dicarboxylate (Intermediate 187A, 1.30 g, 90% purity, 4.64 mmol) in acetone (30 ml) were added 2-bromo-1-(3,4-dichlorophenyl)ethanone (1.37 g, 5.10 mmol) and potassium carbonate (1.92 g, 13.91 mmol). The mixture was stirred for 2 hours at room temperature. After filtering off the solids, the filtrate was concentrated under reduced pressure to provide the crude product. The crude product was suspended in ethyl acetate/petroleum ether (200 ml) (1:20) and stirred for 30 minutes. The solid was collected by filtrated and dried to give 2.70 g (96% of theory, 80% purity) of the title compound.

¹H-NMR (300 MHz, DMSO-d₆): δ [ppm]=8.28 (d, 1H), 8.03-7.96 (m, 1H), 7.93-7.88 (m, 1H), 6.17 (s, 2H), 4.30 (q, 2H), 4.21 (q, 2H), 2.11-2.01 (m, 1H), 1.31 (t, 3H), 1.17 (t, 3H), 1.04-0.83 (m, 2H), 0.80-0.55 (m, 2H).

### Intermediate 260A

### Ethyl 3-cyclopropyl-6-(3,4-dichlorophenyl)-4-oxo-4,5-dihydropyrazolo[1,5-a]pyrazine-2-carboxylate

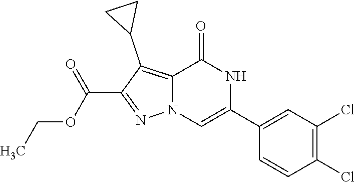 [SMILES: CCOC(=O)c1nn2cc(-c3ccc(Cl)c(Cl)c3)[nH]c(=O)c2c1C1CC1]

Ammonium acetate (9.39 g, 156.73 mmol) was added to a solution of diethyl 4-cyclopropyl-1-[2-(3,4-dichlorophenyl)-2-oxoethyl]-1H-pyrazole-3,5-dicarboxylate (Intermediate 259A, 2.70 g, 5.22 mmol, 85% purity) in acetic acid (150 ml) and the resulting mixture was heated to 110° C. overnight. After cooling to RT, the reaction mixture was poured into ice-water, the solid was collected by filtration, washed with water and dried in vacuo to afford the title compound (2.20 g, 96% of theory, 90% purity).

¹H-NMR (400 MHz, DMSO-d₆): δ [ppm]=8.22 (s, 1H), 8.08-8.03 (m, 1H), 7.78-7.71 (m, 2H), 4.33 (q, 2H), 2.71-2.61 (m, 1H), 1.33 (t, 3H), 1.25-1.16 (m, 2H), 0.99-0.89 (m, 2H).

### Intermediate 261A

### 3-Cyclopropyl-6-(3,4-dichlorophenyl)-4-oxo-4,5-dihydropyrazolo[1,5-a]pyrazine-2-carboxylic acid

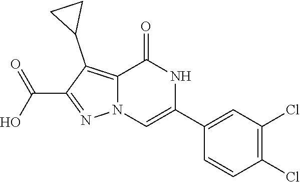 [SMILES: O=C(O)c1nn2cc(-c3ccc(Cl)c(Cl)c3)[nH]c(=O)c2c1C1CC1]

Sodium hydroxide (2.02 g, 50.48 mmol, dissolved in 15 ml water) was added to a solution of ethyl 3-cyclopropyl-6-(3,4-dichlorophenyl)-4-oxo-4,5-dihydropyrazolo[1,5-a]pyrazine-2-carboxylate (Intermediate 260A, 2.20 g, 90% purity, 5.05 mmol) in ethanol (45 ml). After stirring at room temperature for 2 hours, the reaction mixture was concentrated under reduce pressure to remove the ethanol. Then, the rest of mixture was diluted with water (150 ml) and acidified to pH 1 by addition of 2N hydrochloric acid. The solid was collected by filtration, washed with water and dried in air to give the title compound (1.85 g, 99% of theory).

¹H-NMR (400 MHz, DMSO-d₆): δ [ppm]=11.46 (br s, 1H), 8.17 (br s, 1H), 8.04 (br s, 1H), 7.74 (br s, 2H), 2.99-2.73 (m, 1H), 1.32-1.13 (m, 2H), 0.92-0.66 (m, 2H).

### Intermediate 262A

### (S)—N-[(4-Fluorophenyl)methylene]-2-methyl-propane-2-sulfinamide

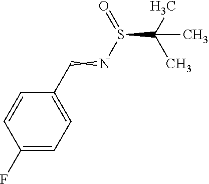

To a solution of 4-fluorobenzaldehyde (30 g, 241.72 mmol) and CuSO₄ (120 g, 751.8 mmol) in DCM (300 mL), were added 2-methylpropane-2-sulfinamide (29.3 g, 241.7 mmol) and PTSA (1.5 g, 8.7 mmol) and the mixture was stirred at 30° C. for 60 h under N₂ atmosphere. The reaction mixture was filtered and the filter cake was washed with DCM. The filtrate was washed with saturated Na—HCO₃ solution (120 mL) and saturated NaCl solution (50 mL), dried over anhydrous Na₂SO₄, filtered and concentrated under reduced pressure to give (S)—N-[(4-fluorophenyl)methylene]-2-methyl-propane-2-sulfinamide (44.5 g, 195.7 mmol, 81% of theory) as a viscous yellow oil.

¹H-NMR (400 MHz, CHCl₃-d): δ [ppm]=8.55 (s, 1H), 7.87 (m, 2H), 7.17 (m, 2H), 1.69 (s, 1H), 1.27 (s, 9H).

### Intermediate 263A

### Ethyl (3S)-3-[[(S)-tert-butylsulfinyl]amino]-2,2-difluoro-3-(4-fluorophenyl)propanoate

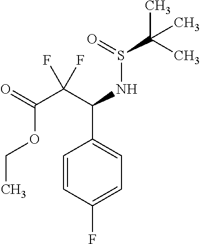 [SMILES: CCOC(=O)C(F)(F)[C@@H](N[S@@](=O)C(C)(C)C)c1ccc(F)cc1]

A suspension of Zn (153.0 g, 2.3 mol) in THF (600 mL) was stirred in 30° C. at N₂ atmosphere. TMSCl (6.8 g, 63 mmol) was added and the mixture was stirred for 15 min. Then ethyl 2-bromo-2,2-difluoro-acetate (236.6 g, 1.17 mol, 149.8 mL) in THF (700 mL) was added dropwise and the temperature of the reaction was kept below 50° C. The mixture was stirred and cooled to room temperature and (S)—N-[(4-fluorophenyl)methylene]-2-methyl-propane-2-sulfinamide (Intermediate 262A, 53 g, 233.2 mmol) in THF (200 mL) was added. The mixture was stirred at 30° C. for 20 h under N₂ atmosphere. The mixture was filtered and the filtrate was concentrated, quenched with saturated solution of NH₄Cl (1600 mL), washed with brine (1000 mL) and dried with anhydrous Na₂SO₄. The mixture was purified by column chromatography on silica gel (eluent: gradient of ethyl acetate in cyclohexane) to afford the title compound as yellow oil with 86% purity. The compound was purified for second time by column chromatography on silica gel (eluent: gradient of ethyl acetate in cyclohexane), to give ethyl (3S)-3-[[(S)-tert-butylsulfinyl]amino]-2,2-difluoro-3-(4-fluorophenyl)propanoate (47.7 g, 135.8 mmol) as yellow oil.

¹H-NMR (400 MHz, CHCI₃-d): δ [ppm]=7.37 (m, 2H), 7.06-7.10 (m, 2H), 5.01-4.94 (dd, 1H), 4.4 (m, 1H), 4.26 (m, 2H), 1.29 (dd, 3H), 1.24 (s, 9H).

### Intermediate 264A

### Ethyl (3S)-3-amino-2,2-difluoro-3-(4-fluorophenyl)propanoate hydrochloride

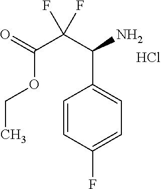 [SMILES: CCOC(=O)C(F)(F)[C@@H](N)c1ccc(F)cc1 ; Cl]

To a solution of ethyl (3S)-3-[[(S)-tert-butylsulfinyl]amino]-2,2-difluoro-3-(4-fluorophenyl) propanoate (Intermediate 263A, 46 g, 130.9 mmol) in EtOH (300 mL) was added HCl/dioxane (4 M, 350 mL) and the mixture was stirred at 30° C. for 2h. The mixture was concentrated under reduced pressure. Then 500 mL MTBE was added and the suspension was stirred for 1 h and filtered to to give the title compound (29.3 g, 102.3 mmol, 78% of theory, 99% purity, 91.9% ee) as white solid. From this, a part was recrystallized (14.2 g, 50.1 mmol) from a mixture of ethyl acetate/methanol (1.5:1, 125 mL) to give ethyl (3S)-3-amino-2,2-difluoro-3-(4-fluorophenyl)propanoate (3.28 g, 11.6 mmol, 23% of theory, 96.7% ee) as white solid.

¹H-NMR (400 MHz, DMSO-d₆): δ [ppm]=9.42-9.38 (br s, 2H), 7.61 (br s, 2H), 7.39-7.33 (m, 2H), 5.36-5.29 (m, 1H), 4.24-4.19 (dd, 2H), 1.12 (dd, 3H).

### Intermediate 265A

### (3S)-3-Amino-2,2-difluoro-3-(4-fluorophenyl)propan-1-ol

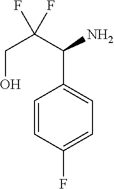 [SMILES: N[C@@H](c1ccc(F)cc1)C(F)(F)CO]

To a suspension of LiBH₄ (1.43 g, 65.6 mmol,) in THF (100 mL) at 0° C., was added ethyl (3S)-3-amino-2,2-difluoro-3-(4-fluorophenyl)propanoate (Intermediate 264A, 6.2 g, 21.8 mmol). The mixture was warmed to rt and stirred for 2 hours. The reaction mixture was poured to a solution of NH₄OH (20 mL) and H₂O (50 mL). The mixture was stirred for 1 h, then extracted with ethyl acetate (3×150 mL), washed with saturated NaCl (100 mL), dried over Na₂SO₄, filtered and the filtrate was evaporated in vacuo. The residue was purified by preparative HPLC (Method P15) to give tittle compound (3.5 g, 16.4 mmol, 77.56% yield, 96% of theory) as colorless oil.

¹H-NMR (400 MHz, DMSO-d₆): δ [ppm]=7.46-7.42 (dd, 2H), 7.18-7.14 (m, 2H), 5.46 (br s, 1H), 4.28-4.22 (m, 1H), 3.77 (m, 1H), 3.54 (m, 1H), 2.21 (br s, 2H).

### Intermediate 266A

### Diethyl 1-[2-(4-chloro-3-methylphenyl)-2-oxoethyl]-4-cyclopropyl-1H-pyrazole-3,5-dicarboxylate

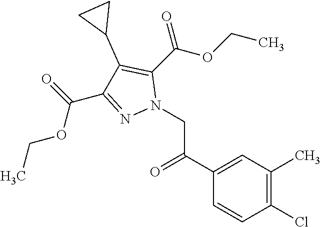 [SMILES: CCOC(=O)c1nn(CC(=O)c2ccc(Cl)c(C)c2)c(C(=O)OCC)c1C1CC1]

To a solution of diethyl 4-cyclopropyl-1H-pyrazole-3,5-dicarboxylate (Intermediate 187A, 2.5 g, 9.91 mmol) in acetone (87 ml) were added 2-bromo-1-(4-chloro-3-methylphenyl)ethanone (3.0 g, 11.89 mmol, 97% purity) and potassium carbonate (3.4 g, 24.77 mmol). The mixture was stirred for 1 hour at room temperature. After filtering off the solids, the filtrate was concentrated under reduced pressure to give 5.05 g (100% of theory, 83% purity) of the title compound. LC/MS [Method 3]: Rₜ=2.38 min; MS (ESIpos): m/z=419 [M+H]⁺.

### Intermediate 267A

### Ethyl 6-(4-chloro-3-methylphenyl)-3-cyclopropyl-4-oxo-4,5-dihydropyrazolo[1,5-a]pyrazine-2-carboxylate

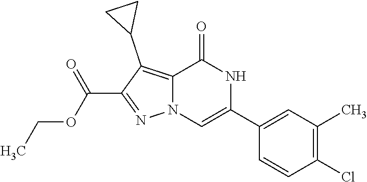 [SMILES: CCOC(=O)c1nn2cc(-c3ccc(Cl)c(C)c3)[nH]c(=O)c2c1C1CC1]

Ammonium acetate (7.71 g, 100 mmol) was added to a solution of diethyl 1-[2-(4-chloro-3-methylphenyl)-2-oxoethyl]-4-cyclopropyl-1H-pyrazole-3,5-dicarboxylate (Intermediate 266A, 5.05 g, 10 mmol, 83% purity) in acetic acid (42 ml) and the resulting mixture was heated to 110° C. for 3 days. After cooling to room temperature, the reaction mixture was poured into ice-water, the solid was collected by filtration, washed with water and dried in vacuo to afford the title compound (3.29 g, 88% of theory).

LC/MS [Method 3]: Rₜ=2.16 min; MS (ESIpos): m/z=372 [M+H]⁺.

### Intermediate 268A

### 6-(4-Chloro-3-methylphenyl)-3-cyclopropyl-4-oxo-4,5-dihydropyrazolo[1,5-a]pyrazine-2-carboxylic acid

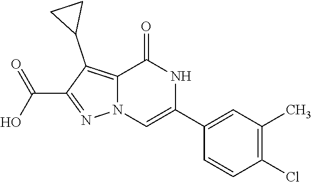 [SMILES: Cc1cc(-c2cn3nc(C(=O)O)c(C4CC4)c3c(=O)[nH]2)ccc1Cl]

Lithium hydroxide (51.48 ml of a 1M solution,51.49 mmol) was added to a solution of ethyl 6-(4-chloro-3-methylphenyl)-3-cyclopropyl-4-oxo-4,5-dihydropyrazolo[1,5-a]pyrazine-2-carboxylate (Intermediate 267A, 3.82 g, 10.23 mmol) in a mixture THF/methanol 5/1 (78 ml). After stirring at room temperature for 6 hours, the reaction mixture was concentrated under reduced pressure.

The residue was diluted with water and the pH value of the mixture was adjusted to 3-4 with hydrochloric acid. The precipitate was collected by filtration, washed with water and dried in air to give 2.66 g (75% of theory) of the title compound.

LC/MS [Method 3]: Rₜ=1.61 min; MS (ESIpos): m/z=344 [M+H]⁺.

¹H-NMR (400 MHz, DMSO-d₆): δ [ppm]=12.96-13.23 (br s, 1H), 11.44 (s, 1H), 8.04 (s, 1H), 7.78 (s, 1H), 7.60 (dd, 1H), 7.52 (d, 1H), 2.73 (m, 1H), 2.38 (s, 3H), 1.19-1.30 (m, 2H), 0.86-0.99 (m, 2H).

### Intermediate 269A

### 2-Bromo-1-(4-chloro-3,5-difluorophenyl)ethan-1-one

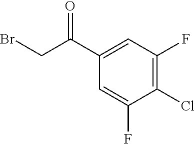 [SMILES: O=C(CBr)c1cc(F)c(Cl)c(F)c1]

To a solution of 1-(4-chloro-3,5-difluorophenyl)ethan-1-one (5.25 g, 27.5 mmol) in THF (53 ml) was added phenyltrimethylammonium tribromide (10.4 g, 27.5 mmol) in portions at RT and the mixture was stirred over night at this temperature. The insoluble material was filtered off and the filtrate was evaporated to leave the crude product (10.3 g, quant., 75% purity) which was used in the next step without further purification.

¹H-NMR (400 MHz, DMSO-d₆): δ [ppm]=7.96-7.91 (m, 2H), 4.97 (s, 2H).

### Intermediate 270A

### Diethyl 1-[2-(4-chloro-3,5-difluorophenyl)-2-oxoethyl]-4-cyclopropyl-1H-pyrazole-3,5-dicarboxylate

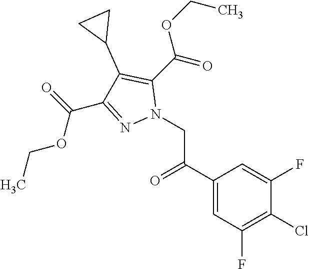 [SMILES: CCOC(=O)c1nn(CC(=O)c2cc(F)c(Cl)c(F)c2)c(C(=O)OCC)c1C1CC1]

2-Bromo-1-(4-chloro-3,5-difluorophenyl)ethan-1-one (5.20 g, 75% purity, 14.5 mmol, Intermediate 269A) and potassium carbonate (4.17 g, 30.2 mmol) were added to a mixture of diethyl 4-cyclopropyl-1H-pyrazole-3,5-dicarboxylate (Intermediate 187A, 3.04 g, 12.1 mmol) in acetone (95 ml) and the mixture was stirred at RT overnight. The insoluble material was filtered off, the solids were washed with acetone and the combined filtrates were evaporated to leave the crude product (7.67 g, quant, 84% purity) which was used in the next step without further purification.

LC/MS [Method 3]: Rₜ=2.35 min; MS (ESIpos): m/z=441 [M+H]⁺.

### Intermediate 271A

### Ethyl 6-(4-chloro-3,5-difluorophenyl)-3-cyclopropyl-4-oxo-4,5-dihydropyrazolo[1,5-a]pyrazine-2-carboxylate

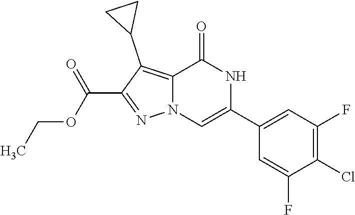 [SMILES: CCOC(=O)c1nn2cc(-c3cc(F)c(Cl)c(F)c3)[nH]c(=O)c2c1C1CC1]

A mixture of diethyl 1-[2-(4-chloro-3,5-difluorophenyl)-2-oxoethyl]-4-cyclopropyl-1H-pyrazole-3,5-dicarboxylate (Intermediate 270A, 7.67 g, 17.4 mmol) and ammonium acetate (53.6 g, 696 mmol) in acetic acid (120 ml) was heated to 110° C. overnight. After cooling to RT, the mixture was poured into water (200 ml) and the precipitate was collected by filtration, washed with water and MTBE (50 ml) and dried to afford the title compound (4.25 g, 62% of theory).

LC/MS [Method 3]: Rₜ=2.11 min; MS (ESIpos): m/z=394 [M+H]⁺.

¹H-NMR (400 MHz, DMSO-d₆): δ [ppm]=11.55 (br s, 1H), 8.34 (s, 1H), 7.84-7.79 (m, 2H), 4.34 (q, 2H), 2.68-2.61 (m, 1H), 1.33 (t, 3H), 1.26-1.17 (m, 2H), 0.99-0.93 (m, 2H).

### Intermediate 272A

### 6-(4-Chloro-3,5-difluorophenyl)-3-cyclopropyl-4-oxo-4,5-dihydropyrazolo[1,5-a]pyrazine-2-carboxylic acid

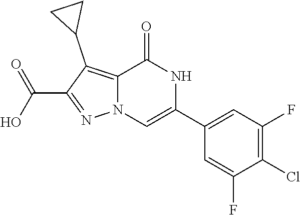 [SMILES: O=C(O)c1nn2cc(-c3cc(F)c(Cl)c(F)c3)[nH]c(=O)c2c1C1CC1]

A mixture of ethyl 6-(4-chloro-3,5-difluorophenyl)-3-cyclopropyl-4-oxo-4,5-dihydropyrazolo[1,5-a]-pyrazine-2-carboxylate (Intermediate 271A, 4.25 g, 10.8 mmol) and lithium hydroxide (1.29 g, 54.0 mmol) in water (54 ml) and ethanol (110 ml) was stirred at RT overnight. The ethanol was distilled off under reduced pressure and the residue was diluted with water and acidified by addition of 1.0 M hydrochloric acid. The precipitate was collected by filtration and dried to afford the title compound. Yield: 3.85 g (98% of theory).

LC/MS [Method 7]: Rₜ=0.86 min; MS (ESIneg): m/z=364 [M−H]⁻.

¹H-NMR (400 MHz, DMSO-d₆): δ [ppm]=13.18 (br s, 1H), 11.51 (br s, 1H), 8.28 (s, 1H), 7.86-7.80 (m, 2H), 2.77-2.66 (m, 1H), 1.33-1.16 (m, 2H), 1.06-0.84 (m, 2H).

### Intermediate 273A

### Diethyl 1-[2-(4-chloro-3-fluorophenyl)-2-oxoethyl]-4-(propan-2-yl)-1H-pyrazole-3,5-dicarboxylate

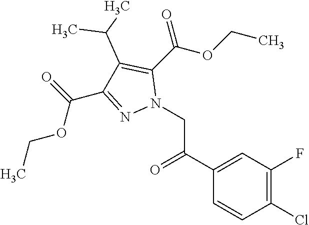 [SMILES: CCOC(=O)c1nn(CC(=O)c2ccc(Cl)c(F)c2)c(C(=O)OCC)c1C(C)C]

A mixture of diethyl 4-(propan-2-yl)-1H-pyrazole-3,5-dicarboxylate (Intermediate 16A, 1.10 g, 4.33 mmol), 2-bromo-1-(4-chloro-3-fluorophenyl)ethan-1-one (Intermediate 255A, 1.31 g, 5.19 mmol) and potassium carbonate (1.49 g, 10.8 mmol) in acetone (44 ml) was stirred at RT overnight. The insoluble material was filtered off, the solids were washed with acetone and the combined filtrates were evaporated to leave the crude product (1.90 g, 93% of theory, 90% purity) which was used in the next step without further purification.

LC/MS [Method 3]: Rₜ=2.40 min; MS (ESIpos): m/z=425 [M+H]⁺.

### Intermediate 274A

### Ethyl 6-(4-chloro-3-fluorophenyl)-4-oxo-3-(propan-2-yl)-4,5-dihydropyrazolo[1,5-a]pyrazine-2-carboxylate

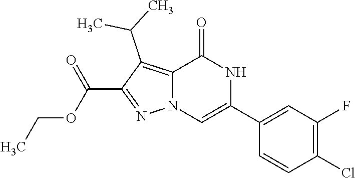 [SMILES: CCOC(=O)c1nn2cc(-c3ccc(Cl)c(F)c3)[nH]c(=O)c2c1C(C)C]

A mixture of diethyl 1-[2-(4-chloro-3-fluorophenyl)-2-oxoethyl]-4-(propan-2-yl)-1H-pyrazole-3,5-dicarboxylate (Intermediate 273A, 2.00 g, 90% purity, 4.24 mmol) and ammonium acetate (13.1 g, 169 mmol) in acetic acid (54 ml) was heated to 110° C. overnight. After cooling to RT, the mixture was poured into water (100 ml) and the precipitate was collected by filtration, washed with water and dried to afford the title compound (1.80 g, 98% of theory, 87% purity). LC/MS [Method 3]: Rₜ=2.15 min; MS (ESIpos): m/z=378 [M+H]⁺.

¹H-NMR (600 MHz, DMSO-d₆): δ [ppm]=11.64 (br s, 1H), 8.26 (s, 1H), 7.87 (dd, 1H), 7.77-7.69 (m, 1H), 7.65 (dd, 1H), 4.34 (q, 2H), 4.12 (spt, 1H), 1.36 (d, 6H), 1.33 (t, 3H).

### Intermediate 275A

### 6-(4-Chloro-3-fluorophenyl)-4-oxo-3-(propan-2-yl)-4,5-dihydropyrazolo[1,5-a]pyrazine-2-carboxylic acid

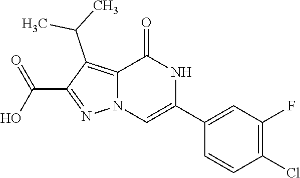 [SMILES: CC(C)c1c(C(=O)O)nn2cc(-c3ccc(Cl)c(F)c3)[nH]c(=O)c12]

A mixture of ethyl 6-(4-chloro-3-fluorophenyl)-4-oxo-3-(propan-2-yl)-4,5-dihydropyrazolo[1,5-a]pyrazine-2-carboxylate (Intermediate 274A, 1.80 g, 87% purity, 4.14 mmol) and lithium hydroxide (993 mg, 41.4 mmol) in water (22 ml) and ethanol (43 ml) was stirred at RT for three days. The ethanol was distilled off under reduced pressure and the residue was diluted with water and acidified by addition of 1.0 M hydrochloric acid. The precipitate was collected by filtration and dried to afford the title compound. Yield: 1.45 g (98% of theory).

LC/MS [Method 3]: Rₜ=1.64 min; MS (ESIneg): m/z=348 [M−H]⁻.

¹H-NMR (400 MHz, DMSO-d₆): δ [ppm]=13.17 (br s, 1H), 11.59 (s, 1H), 8.19 (s, 1H), 7.88 (dd, 1H), 7.75-7.68 (m, 1H), 7.68-7.63 (m, 1H), 4.12 (spt, 1H), 1.36 (d, 6H).

### Intermediate 276A

### 2-Bromo-1-(4-chloro-3-methoxyphenyl)ethan-1-one

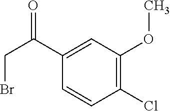

To a solution of 1-(4-chloro-3-methoxyphenyl)ethan-1-one (4.34 g, 23.5 mmol) in THF (38 ml) was added phenyltrimethylammonium tribromide (8.84 g, 23.5 mmol) in portions at RT and the mixture was stirred at this temperature for two days. The insoluble material was filtered off and the filtrate was evaporated to leave the crude product (9.42 g, quant., 66% purity) which was used in the next step without further purification.

LC/MS [Method 3]: Rₜ=1.86 min; MS (ESIpos): m/z=263 [M+H]⁺.

### Intermediate 277A

### Diethyl 1-[2-(4-chloro-3-methoxyphenyl)-2-oxoethyl]-4-cyclopropyl-1H-pyrazole-3,5-dicarboxylate

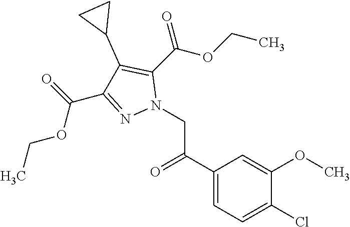 [SMILES: CCOC(=O)c1nn(CC(=O)c2ccc(Cl)c(OC)c2)c(C(=O)OCC)c1C1CC1]

A mixture of diethyl 4-cyclopropyl-1H-pyrazole-3,5-dicarboxylate (Intermediate 187A, 4.94 g, 19.6 mmol), 2-bromo-1-(4-chloro-3-methoxyphenyl)ethan-1-one (Intermediate 276A, 6.19 g, 23.5 mmol) and potassium carbonate (4.06 g, 29.4 mmol) in acetone (150 ml) was stirred at RT overnight. The insoluble material was filtered off, the solids were washed with acetone and the combined filtrates were evaporated to leave the crude product (12.8 g, 99% of theory, 66% purity) which was used in the next step without further purification.

LC/MS [Method 3]: Rₜ=2.26 min; MS (ESIpos): m/z=435 [M+H]⁺.

### Intermediate 278A

### Ethyl 6-(4-chloro-3-methoxyphenyl)-3-cyclopropyl-4-oxo-4,5-dihydropyrazolo[1,5-a]pyrazine-2-carboxylate

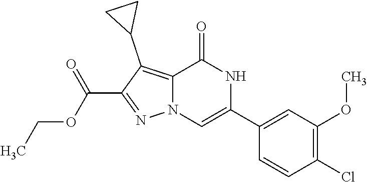 [SMILES: CCOC(=O)c1nn2cc(-c3ccc(Cl)c(OC)c3)[nH]c(=O)c2c1C1CC1]

A mixture of diethyl 1-[2-(4-chloro-3-methoxyphenyl)-2-oxoethyl]-4-cyclopropyl-1H-pyrazole-3,5-dicarboxylate (Intermediate 277A, 8.52 g, 19.6 mmol) and ammonium acetate (60.4 g, 784 mmol) in acetic acid (150 ml) was heated to 110° C. for three days. After cooling to RT, the mixture was poured into water (800 ml) and the precipitate was collected by filtration, washed with water and dried to afford the title compound (7.60 g, 85% of theory, 97% purity).

LC/MS [Method 3]: Rₜ=2.16 min; MS (ESIpos): m/z=388 [M+H]⁺.

### Intermediate 279A

### 6-(4-Chloro-3-methoxyphenyl)-3-cyclopropyl-4-oxo-4,5-dihydropyrazolo[1,5-a]pyrazine-2-carboxylic acid

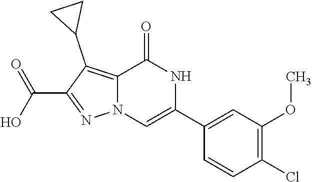 [SMILES: COc1cc(-c2cn3nc(C(=O)O)c(C4CC4)c3c(=O)[nH]2)ccc1Cl]

A mixture of ethyl 6-(4-chloro-3-methoxphenyl)-3-cyclopropyl-4-oxo-4,5-dihydropyrazolo[1,5-a]-pyrazine-2-carboxylate (Intermediate 278A, 7.60 g, 19.6 mmol) and lithium hydroxide (2.35 g, 98.0 mmol) in water (100 ml) and ethanol (200 ml) was stirred at RT for two days. The ethanol was distilled off under reduced pressure and the residue was diluted with water and acidified by addition of 1.0 M hydrochloric acid. The precipitate was collected by filtration and dried to afford the title compound. Yield: 5.37 g (76% of theory).

LC/MS [Method 3]: Rₜ=1.53 min; MS (ESIpos): m/z=360 [M+H]⁺.

¹H-NMR (400 MHz, DMSO-d₆): δ [ppm]=13.10 (br s, 1H), 11.53 (s, 1H), 8.17 (s, 1H), 7.52 (d, 1H), 7.50-7.49 (m, 1H), 7.35 (dd, 1H), 3.97 (s, 3H), 2.78-2.67 (m, 1H), 1.29-1.22 (m, 2H), 0.97-0.89 (m, 2H).

### Intermediate 280A

### Ethyl 4,4,5,5,5-pentafluoro-3-oxo-2-(triphenylphosphoranylidene)pentanoate

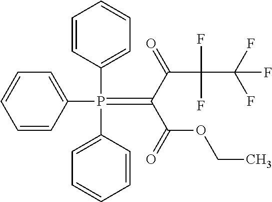 [SMILES: CCOC(=O)C(C(=O)C(F)(F)C(F)(F)F)=P(c1ccccc1)(c1ccccc1)c1ccccc1]

To a solution of ethyl (triphenylphosphoranylidene)acetate (45.00 g, 129.2 mmol) and triethylamine (21.6 mL, 155.0 mmol) in tetrahydrofuran (600 ml) was added pentafluoropropanoic anhydride (44.05 g, 142.1 mmol) at 0° C. After stirring at 0° C. for 2 h, the reaction mixture was quenched with water (500 ml) and extracted with ethyl acetate (3×800 ml). The combined organic layers were washed with brine (2×800 ml) and dried over anhydrous sodium sulfate. The solid was filtered off. The filtrate was concentrated. The residue was triturated with petroleum ether/ethyl acetate (20:1, 200 ml). The solid was collected by filtration and dried under vacuum to give the title compound. Yield: 63.2 g (98% of theory, 99% purity).

LC/MS [Method 40]: Rₜ=1.33 min; MS (ESIpos): m/z=495 [M+H]⁺.

¹H-NMR (400 MHz, CDCl₃): δ [ppm]=7.71-7.49 (m, 15H), 3.85 (q, 2H), 0.95 (t, 3H).

### Intermediate 281A

### Ethyl 4,4,5,5,5-pentafluoropent-2-ynoate

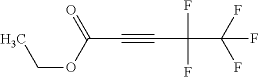

Ethyl 4,4,5,5,5-pentafluoro-3-oxo-2-(triphenylphosphoranylidene)pentanoate (Intermediate 280A, 54.00 g, 109.2 mmol) was placed in a 250-ml round-bottom flask. The solid was heated in a sand bath under reduced pressure (˜23 torr) with a water circulating multi-purpose vacuum pump. Once the distillation pot reached 120° C., the solid phosphorane began to melt and evolution of acetylene started. The mixture was heated to 135˜260° C. and the acetylene was collected in an ethanol-dry ice bath. This resulted in 19.50 g (81% of theory, 99% purity) of the title compound.

¹H-NMR (400 MHz, CDCl₃): δ [ppm]=4.36 (q, 2H), 1.38 (t, 3H).

### Intermediate 282A

### Diethyl 4-(pentafluoroethyl)-1H-pyrazole-3,5-dicarboxylate

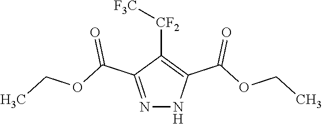 [SMILES: CCOC(=O)c1n[nH]c(C(=O)OCC)c1C(F)(F)C(F)(F)F]

To a solution of ethyl 4,4,5,5,5-pentafluoropent-2-ynoate (Intermediate 281A, 21.20 g, 98.1 mmol) in diethyl ether (200 ml) was added ethyl diazoacetate (11.19 g, 98.1 mmol) at 0° C. under a nitrogen atmosphere. The mixture was stirred overnight at room temperature. The solvent was removed under reduced pressure. The residue was purified by flash chromatography on silica gel (eluent: petroleum ether-ethyl acetate 100%, 88:12) to give 19.00 g (97% of theory, 97% purity) of the title compound.

LC/MS [Method 41]: Rₜ=1.51 min; MS (ESIpos): m/z=331 [M+H]⁺.

¹H-NMR (400 MHz, DMSO-d₆): δ [ppm]=15.30 (s, 1H), 4.35 (q, 4H), 1.30 (t, 6H).

### Intermediate 283A

### Diethyl 1-[2-(3-chloro-4-methylphenyl)-2-oxoethyl]-4-(pentafluoroethyl)-1H-pyrazole-3,5-dicarboxylate

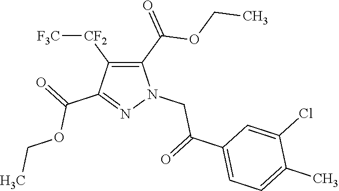 [SMILES: CCOC(=O)c1nn(CC(=O)c2ccc(C)c(Cl)c2)c(C(=O)OCC)c1C(F)(F)C(F)(F)F]

To a solution of diethyl 4-(pentafluoroethyl)-1H-pyrazole-3,5-dicarboxylate (Intermediate 282A, 3.20 g, 9.4 mmol, 97% purity) and 2-bromo-1-(3-chloro-4-methylphenyl)ethanone (2.50 g, 9.4 mmol, 93% purity) in acetone (30 ml) was added potassium carbonate (3.90 g, 28.2 mmol). The resulting mixture was stirred overnight at room temperature. After filtering off the solids, the filter cake was washed with ethyl acetate and the filtrate was concentrated under reduced pressure. The residue was purified by flash chromatography on silica gel (eluent: petroleum ether-ethyl acetate, 9:1) to give 4.70 g (94% of theory, 93% purity) of the title compound.

LC/MS [Method 42]: Rₜ=1.43 min; MS (ESIpos): m/z=497 [M+H]⁺.

¹H-NMR (300 MHz, DMSO-d₆): δ [ppm]=8.07 (d, 1H), 7.91 (dd, 1H), 7.62 (t, 1H), 6.29 (s, 2H), 4.35 (q, 2H), 4.24 (q, 2H), 2.45 (s, 3H), 1.29 (t, 3H), 1.12 (t, 3H).

### Intermediate 284A

### Ethyl 6-(3-chloro-4-methylphenyl)-4-oxo-3-(pentafluoroethyl)-4,5-dihydropyrazolo[1,5-a]pyrazine-2-carboxylate

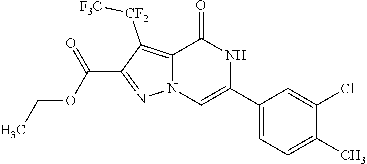 [SMILES: CCOC(=O)c1nn2cc(-c3ccc(C)c(Cl)c3)[nH]c(=O)c2c1C(F)(F)C(F)(F)F]

To a solution of diethyl 1-[2-(3-chloro-4-methylphenyl)-2-oxoethyl]-4-(pentafluoroethyl)-1H-pyrazole-3,5-dicarboxylate (Intermediate 283A, 4.70 g, 8.8 mmol, 93% purity) in acetic acid (60 ml) was added ammonium acetate (27.13 g, 351.9 mmol). The resulting mixture was heated to 110° C. overnight. After cooling to room temperature, the reaction mixture was poured into ice-water. The solid was collected by filtration, washed with water and dried in vacuum to give 4.20 g (99% of theory, 94% purity) of the title compound.

¹H-NMR (300 MHz, DMSO-d₆): δ [ppm]=12.13 (s, 1H), 8.34 (s, 1H), 7.87 (d, 1H), 7.66 (dd, 1H), 7.50 (d, 1H), 4.38 (q, 2H), 2.40 (s, 3H), 1.31 (t, 3H).

### Intermediate 285A

### 6-(3-Chloro-4-methylphenyl)-4-oxo-3-(pentafluoroethyl)-4,5-dihydropyrazolo[1,5-a]pyrazine-2-carboxylic acid

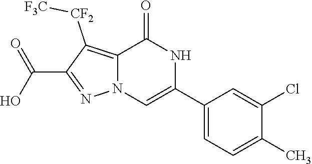 [SMILES: Cc1ccc(-c2cn3nc(C(=O)O)c(C(F)(F)C(F)(F)F)c3c(=O)[nH]2)cc1Cl]

To a solution of ethyl 6-(3-chloro-4-methylphenyl)-4-oxo-3-(pentafluoroethyl)-4,5-dihydropyrazolo-[1,5-a]pyrazine-2-carboxylate (Intermediate 284A, 4.10 g, 8.6 mmol, 94% purity) in ethanol (60 ml) was added a solution of sodium hydroxide (3.43 g, 85.7 mmol) in water (20 ml). After stirring overnight at 40° C., the reaction mixture was concentrated under reduced pressure to remove ethanol. Then, the remaining mixture was diluted with water (20 ml) and extracted with methyl tert-butyl ether (2×50 ml). The aqueous layer was acidified with 2M hydrochloric acid to pH=1. The product was collected by filtration, washed with water and dried in air to give 3.59 g (96% of theory) of the title compound.

LC/MS [Method 43]: Rₜ=1.68 min; MS (ESIpos): m/z=422 [M+H]⁺.

¹H-NMR (400 MHz, DMSO-d₆): δ [ppm]=11.99 (s, 1H), 8.22 (s, 1H), 7.86 (s, 1H), 7.65 (d, 1H), 7.48 (d, 1H), 2.38 (d, 3H).

### Intermediate 286A

### Diethyl 1-[2-(3-fluoro-4-methylphenyl)-2-oxoethyl]-4-(pentafluoroethyl)-1H-pyrazole-3,5-dicarboxylate

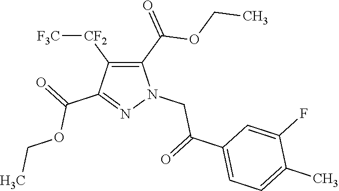 [SMILES: CCOC(=O)c1nn(CC(=O)c2ccc(C)c(F)c2)c(C(=O)OCC)c1C(F)(F)C(F)(F)F]

This compound was synthesized by the same method as described for Intermediate 283A except that 2-bromo-1-(3-fluoro-4-methylphenyl)ethanone (2.24 g, 8.8 mmol, 91% purity) was used.

Yield: 3.00 g (69% of theory, 97% purity).

LC/MS [Method 12]: Rₜ=1.25 min; MS (ESIpos): m/z=481 [M+H]⁺.

¹H-NMR (300 MHz, DMSO-d₆): δ [ppm]=7.83-7.79 (m, 2H), 7.57 (t, 1H), 6.28 (s, 2H), 4.37 (q, 2H), 4.25 (q, 2H), 2.37 (d, 3H), 1.30 (t, 3H), 1.13 (t, 3H).

### Intermediate 287A

### Ethyl 6-(3-fluoro-4-methylphenyl)-4-oxo-3-(pentafluoroethyl)-4,5-dihydropyrazolo[1,5-a]pyrazine-2-carboxylate

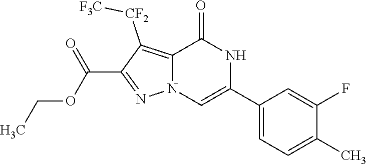 [SMILES: CCOC(=O)c1nn2cc(-c3ccc(C)c(F)c3)[nH]c(=O)c2c1C(F)(F)C(F)(F)F]

This compound was synthesized by the same method as described for Intermediate 284A except that diethyl 1-[2-(3-fluoro-4-methylphenyl)-2-oxoethyl]-4-(pentafluoroethyl)-1H-pyrazole-3,5-dicarboxylate (Intermediate 286A, 3.00 g, 6.1 mmol, 97% purity) was used. Yield: 2.50 g (86% of theory, 90% purity).

LC/MS [Method 44]: Rₜ=1.13 min; MS (ESIpos): m/z=434 [M+H]⁺.

¹H-NMR (300 MHz, DMSO-d6): δ [ppm]=12.12 (s, 1H), 8.34 (s, 1H), 7.65-7.41 (m, 3H), 4.39 (q, 2H), 2.30 (d, 3H), 1.32 (t, 3H).

### Intermediate 288A

### 6-(3-Fluoro-4-methylphenyl)-4-oxo-3-(pentafluoroethyl)-4,5-dihydropyrazolo[1 ,5-a]pyrazine-2-carboxylic acid

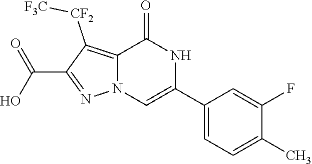 [SMILES: Cc1ccc(-c2cn3nc(C(=O)O)c(C(F)(F)C(F)(F)F)c3c(=O)[nH]2)cc1F]

This compound was synthesized by the same method as described for Intermediate 285A except that ethyl 6-(3-fluoro-4-methylphenyl)-4-oxo-3-(pentafluoroethyl)-4,5-dihydropyrazolo[1,5-a]-pyrazine-2-carboxylate (Intermediate 287A, 2.40 g, 5.0 mmol, 90% purity) was used. Yield: 1.91 g (94% of theory, 99% purity).

LC/MS [Method 43]: Rₜ=1.60 min; MS (ESIpos): m/z=406 [M+H]⁺.

¹H-NMR (400 MHz, DMSO-d6): δ [ppm]=11.91 (s, 1H), 8.23-8.19 (m, 1H), 7.61 (dd, 1H), 7.54 (dd, 1H), 7.40 (d, 1H), 2.33 (d, 3H).

### Intermediate 289A

### Diethyl 1-[2-(3,4-dimethylphenyl)-2-oxoethyl]-4-(pentafluoroethyl)-1H-pyrazole-3,5-dicarboxylate

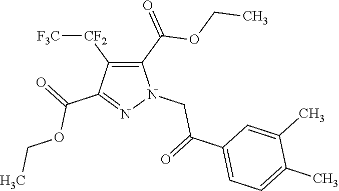 [SMILES: CCOC(=O)c1nn(CC(=O)c2ccc(C)c(C)c2)c(C(=O)OCC)c1C(F)(F)C(F)(F)F]

This compound was synthesized by the same method as described for Intermediate 283A except that 2-bromo-1-(3,4-dimethylphenyl)ethanone (Intermediate 20A, 2.50 g, 8.8 mmol, 80% purity) was used. Yield: 3.30 g (77% of theory, 98% purity).

LC/MS [Method 44]: Rₜ=1.26 min; MS (ESIpos): m/z=477 [M+H]⁺.

¹H-NMR (300 MHz, DMSO-d₆): δ [ppm]=7.83-7.77 (m, 2H), 7.38 (d, 1H), 6.23 (s, 2H), 4.37 (q, 2H), 4.25 (q, 2H), 2.33 (d, 6H), 1.31 (t, 3H), 1.14 (t, 3H).

### Intermediate 290A

### Ethyl 6-(3,4-dimethylphenyl)-4-oxo-3-(pentafluoroethyl)-4,5-dihydropyrazolo[1,5-a]pyrazine-2-carboxylate

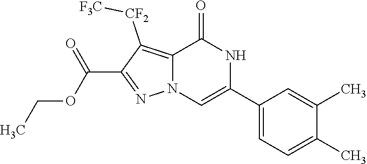 [SMILES: CCOC(=O)c1nn2cc(-c3ccc(C)c(C)c3)[nH]c(=O)c2c1C(F)(F)C(F)(F)F]

This compound was synthesized by the same method as described for Intermediate 284A except that diethyl 1-[2-(3,4-dimethylphenyl)-2-oxoethyl]-4-(pentafluoroethyl)-1H-pyrazole-3,5-dicarboxylate (Intermediate 289A, 3.30 g, 6.8 mmol, 98% purity) was used. Yield: 3.20 g (93% of theory, 85% purity).

LC/MS [Method 12]: Rₜ=1.22 min; MS (ESIpos): m/z=430 [M+H]⁺.

¹H-NMR (300 MHz, DMSO-d₆): δ [ppm]=12.02 (s, 1H), 8.20 (s, 1H), 7.58 (d, 1H), 7.50 (dd, 1H), 7.27 (d, 1H), 4.38 (q, 2H), 2.29 (s, 3H), 2.28 (s, 3H), 1.31 (t, 3H).

### Intermediate 291A

### 6-(3,4-Dimethylphenyl)-4-oxo-3-(pentafluoroethyl)-4,5-dihydropyrazolo[1,5-a]pyrazine-2-carboxylic acid

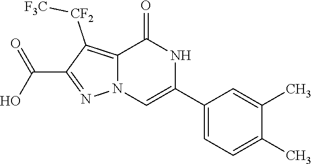 [SMILES: Cc1ccc(-c2cn3nc(C(=O)O)c(C(F)(F)C(F)(F)F)c3c(=O)[nH]2)cc1C]

This compound was synthesized by the same method as described for Intermediate 285A except that ethyl 6-(3,4-dimethylphenyl)-4-oxo-3-(pentafluoroethyl)-4,5-dihydropyrazolo[1,5-a]pyrazine-2-carboxylate (Intermediate 290A, 3.10 g, 6.1 mmol, 85% purity) was used. Yield: 2.47 g (95% of theory, 95% purity).

LC/MS [Method 43]: Rₜ=1.63 min; MS (ESIpos): m/z=402 [M+H]⁺.

¹H-NMR (400 MHz, DMSO-d₆): δ [ppm]=11.90 (s, 1H), 8.10 (s, 1H), 7.57 (s, 1H), 7.49 (dd, 1H), 7.26 (d, 1H), 2.29 (s, 3H), 2.28 (s, 3H).

### Intermediate 292A

### Diethyl 1-[2-(4-chloro-3-methylphenyl)-2-oxoethyl]-4-(pentafluoroethyl)-1H-pyrazole-3,5-dicarboxylate

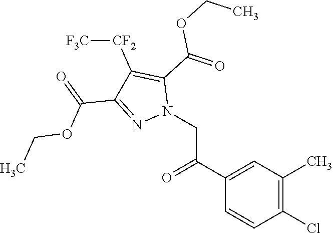 [SMILES: CCOC(=O)c1nn(CC(=O)c2ccc(Cl)c(C)c2)c(C(=O)OCC)c1C(F)(F)C(F)(F)F]

Potassium carbonate (1.05 g, 7.57 mmol) was added to a mixture of diethyl 4-(pentafluoroethyl)-1H-pyrazole-3,5-dicarboxylate (Intermediate 282A, 1.00 g, 3.03 mmol) and 2-bromo-1-(4-chloro-3-methylphenyl)ethan-1-one (1.20 g, 75% purity, 3.63 mmol) in acetone (24 ml). After stirring at RT overnight, the mixture was filtered and the filter cake was washed with acetone. The combined filtrates were evaporated and dried under reduced pressure to afford the title compound. Yield: 1.90 g (quant., 96% purity).

LC/MS [Method 7]: Rₜ=1.31 min; MS (ESIpos): m/z=497/499 [M+H]⁺.

¹H-NMR (400 MHz, DMSO-d₆): δ [ppm]=8.04 (d, 1H), 7.87 (dd, 1H), 7.67 (d, 1H), 6.26 (s, 2H), 4.34 (q, 2H), 4.24 (q, 2H), 2.44 (s, 3H), 1.29 (t, 3H), 1.12 (t, 3H).

### Intermediate 293A

### Ethyl 6-(4-chloro-3-methylphenyl)-4-oxo-3-(pentafluoroethyl)-4,5-dihydropyrazolo[1,5-a]pyrazine-2-carboxylate

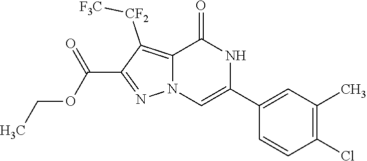 [SMILES: CCOC(=O)c1nn2cc(-c3ccc(Cl)c(C)c3)[nH]c(=O)c2c1C(F)(F)C(F)(F)F]

A mixture of diethyl 1-[2-(4-chloro-3-methylphenyl)-2-oxoethyl]-4-(pentafluoroethyl)-1H-pyrazole-3,5-dicarboxylate (Intermediate 292A, 1.90 g, 3.82 mmol) and ammonium acetate (11.8 g, 153 mmol) in acetic acid (27 ml) was heated to 110° C. overnight. After cooling to RT, the mixture was poured into water (200 ml). The precipitate was collected by filtration, washed with water and MTBE and dried to afford the title compound. Yield: 1.28 g (71% of theory, 95% purity).

LC/MS [Method 3]: Rₜ=2.25 min; MS (ESIpos): m/z=450 [M+H]⁺.

¹H-NMR (400 MHz, DMSO-d₆): δ [ppm]=12.10 (s, 1H), 8.29 (s, 1H), 7.80 (d, 1H), 7.62 (dd, 1H), 7.56 (d, 1H), 4.38 (q, 2H), 2.40 (s, 3H), 1.31 (t, 3H).

### Intermediate 294A

### 6-(4-Chloro-3-methylphenyl)-4-oxo-3-(pentafluoroethyl)-4,5-dihydropyrazolo[1,5-a]pyrazine-2-carboxylic acid

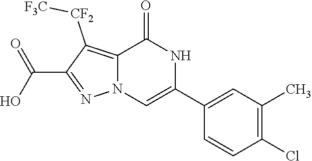 [SMILES: Cc1cc(-c2cn3nc(C(=O)O)c(C(F)(F)C(F)(F)F)c3c(=O)[nH]2)ccc1Cl]

A mixture of ethyl 6-(4-chloro-3-methylphenyl)-4-oxo-3-(pentafluoroethyl)-4,5-dihydropyrazolo-[1,5-a]pyrazine-2-carboxylate (Intermediate 293A, 1.28 g, 2.85 mmol) and lithium hydroxide (341 mg, 14.2 mmol) in water (14 ml) and ethanol (29 ml) was stirred at RT overnight. The ethanol was distilled off under reduced pressure and the residue was diluted with water and acidified by addition of 1.0 M hydrochloric acid. The precipitate was collected by filtration and dried to afford the title compound. Yield: 1.13 g (94% of theory).

LC/MS [Method 7]: Rₜ=0.89 min; MS (ESIpos): m/z=422 [M+H]⁺.

¹H-NMR (400 MHz, DMSO-d₆): δ [ppm]=13.87 (br s, 1H), 12.02 (br s, 1H), 8.22 (s, 1H), 7.84-7.76 (m, 1H), 7.62 (dd, 1H), 7.55 (d, 1H), 2.40 (s, 3H).

### Intermediate 295A

### Diethyl 1-[2-(4-chloro-3-fluorophenyl)-2-oxoethyl]-4-(pentafluoroethyl)-1H-pyrazole-3,5-dicarboxylate

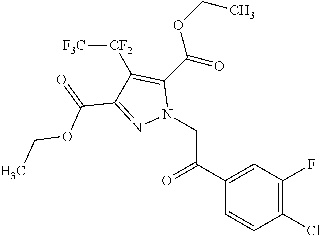 [SMILES: CCOC(=O)c1nn(CC(=O)c2ccc(Cl)c(F)c2)c(C(=O)OCC)c1C(F)(F)C(F)(F)F]

Potassium carbonate (1.05 g, 7.57 mmol) was added to a mixture of diethyl 4-(pentafluoroethyl)-1H-pyrazole-3,5-dicarboxylate (Intermediate 282A, 1.00 g, 3.03 mmol) and 2-bromo-1-(4-chloro-3-fluorophenyl)ethan-1-one (Intermediate 255A, 1.23 g, 74% purity, 3.63 mmol) in acetone (24 ml) and the mixture was stirred at RT overnight. Then, the mixture was filtered and the filter cake was washed with acetone. The combined filtrates were evaporated and dried under reduced pressure to afford the crude title compound which was used in the next step without further purification. Yield: 1.96 g (quant., 94% purity).

LC/MS [Method 3]: Rₜ=2.43 min; MS (ESIneg): m/z=499 [M−H]⁻.

### Intermediate 296A

### Ethyl 6-(4-chloro-3-fluorophenyl)-4-oxo-3-(pentafluoroethyl)-4,5-dihydropyrazolo[1,5-a]pyrazine-2-carboxylate

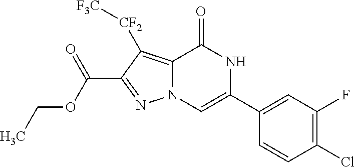 [SMILES: CCOC(=O)c1nn2cc(-c3ccc(Cl)c(F)c3)[nH]c(=O)c2c1C(F)(F)C(F)(F)F]

A mixture of diethyl 1-[2-(4-chloro-3-fluorophenyl)-2-oxoethyl]-4-(pentafluoroethyl)-1H-pyrazole-3,5-dicarboxylate (Intermediate 295A, 1.90 g, 3.79 mmol) and ammonium acetate (11.7 g, 152 mmol) in acetic acid (27 ml) was heated to 110° C. overnight. After cooling to RT, the mixture was poured into water (200 ml). The precipitate was collected by filtration, washed with water and MTBE and dried to afford the title compound. Yield: 1.50 g (87% of theory).

LC/MS [Method 3]: Rₜ=2.14 min; MS (ESIpos): m/z=454 [M+H]⁺.

¹H-NMR (400 MHz, DMSO-d₆): δ [ppm]=12.18 (s, 1H), 8.40 (s, 1H), 7.89 (dd, 1H), 7.81-7.72 (m, 1H), 7.67 (dd, 1H), 4.38 (q, 2H), 1.31 (t, 3H).

### Intermediate 297A

### 6-(4-Chloro-3-fluorophenyl)-4-oxo-3-(pentafluoroethyl)-4,5-dihydropyrazolo[1,5-a]pyrazine-2-carboxylic acid

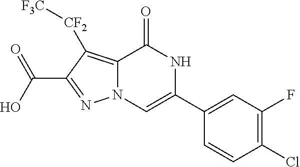 [SMILES: O=C(O)c1nn2cc(-c3ccc(Cl)c(F)c3)[nH]c(=O)c2c1C(F)(F)C(F)(F)F]

A mixture of ethyl 6-(4-chloro-3-fluorophenyl)-4-oxo-3-(pentafluoroethyl)-4,5-dihydropyrazolo-[1,5-a]pyrazine-2-carboxylate (Intermediate 296A, 1.50 g, 3.31 mmol) and lithium hydroxide (396 mg, 16.5 mmol) in water (17 ml) and ethanol (33 ml) was stirred at RT overnight. The ethanol was distilled off under reduced pressure and the residue was diluted with water and acidified by addition of 1.0 M hydrochloric acid. The precipitate was collected by filtration and dried to afford the title compound. Yield: 1.30 g (92% of theory).

LC/MS [Method 3]: Rₜ=1.52 min; MS (ESIpos): m/z=426 [M+H]⁺.

### Intermediate 298A

### Diethyl 1-{2-[3-methyl-4-(trifluoromethyl)phenyl]-2-oxoethyl}-4-(trifluoromethyl)-1H-pyrazole-3,5-dicarboxylate

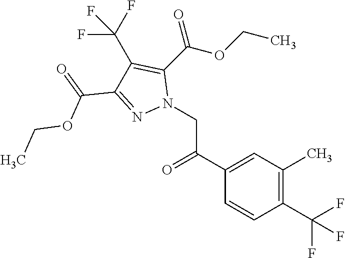 [SMILES: CCOC(=O)c1nn(CC(=O)c2ccc(C(F)(F)F)c(C)c2)c(C(=O)OCC)c1C(F)(F)F]

Potassium carbonate (697 mg, 5.04 mmol) was added to a mixture of diethyl 4-(trifluoromethyl)-1H-pyrazole-3,5-dicarboxylate (Intermediate 32A, 588 mg, 96% purity, 2.02 mmol) and 2-bromo-1-[3-methyl-4-(trifluoromethyl)phenyl]ethan-1-one (Intermediate 188A, 1.00 g, 68% purity, 2.42 mmol) in acetone (22 ml) and the mixture was stirred at RT overnight. Then, the mixture was filtered and the filter cake was washed with acetone. The combined filtrates were evaporated and dried under reduced pressure to afford the crude title compound which was used in the next step without further purification. Yield: 1.20 g (98% of theory, 79% purity).

LC/MS [Method 3]: Rₜ=2.43 min; MS (ESIneg): m/z=479 [M−H]⁻.

### Intermediate 299A

### Ethyl 6-[3-methyl-4-(trifluoromethyl)phenyl]-4-oxo-3-(trifluoromethyl)-4,5-dihydropyrazolo-[1,5-a]pyrazine-2-carboxylate

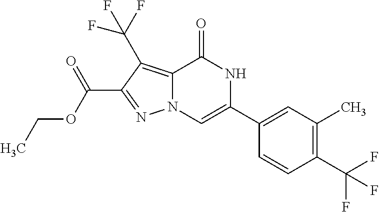 [SMILES: CCOC(=O)c1nn2cc(-c3ccc(C(F)(F)F)c(C)c3)[nH]c(=O)c2c1C(F)(F)F]

A mixture of diethyl 1-{2-[3-methyl-4-(trifluoromethyl)phenyl]-2-oxoethyl}-4-(trifluoromethyl)-1H-pyrazole-3,5-dicarboxylate (Intermediate 298A, 1.20 g, 79% purity, 1.97 mmol) and ammonium acetate (3.80 g, 49.3 mmol) in acetic acid (20 ml) was heated to 110° C. for three days. After cooling to RT, the mixture was poured into water (250 ml). The precipitate was collected by filtration to afford the title compound. Yield: 990 mg (quant, 86% purity).

LC/MS [Method 3]: Rₜ=2.15 min; MS (ESIpos): m/z=434 [M+H]⁺.

¹H-NMR (400 MHz, DMSO-d₆): δ [ppm]=12.22 (s, 1H), 8.40 (s, 1H), 7.89 (s, 1H), 7.83-7.76 (m, 2H), 4.40 (q, 2H), 1.33 (t, 3H).

### Intermediate 300A

### 6-[3-Methyl-4-(trifluoromethyl)phenyl]-4-oxo-3-(trifluoromethyl)-4,5-dihydropyrazolo[1,5-a]-pyrazine-2-carboxylic acid

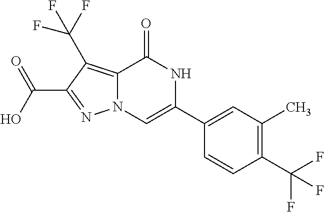 [SMILES: Cc1cc(-c2cn3nc(C(=O)O)c(C(F)(F)F)c3c(=O)[nH]2)ccc1C(F)(F)F]

A mixture of ethyl 6-[3-methyl-4-(trifluoromethyl)phenyl]-4-oxo-3-(trifluoromethyl)-4,5-dihydropyrazolo[1,5-a]pyrazine-2-carboxylate (Intermediate 299A, 990 mg, 86% purity, 1.96 mmol) and lithium hydroxide (235 mg, 9.82 mmol) in water (25 ml) and methanol (100 ml) was stirred at RT overnight. The methanol was distilled off under reduced pressure and the residue was diluted with water and acidified by addition of 1.0 M hydrochloric acid. The precipitate was collected by filtration and dried to afford the title compound. Yield: 710 mg (89% of theory).

LC/MS [Method 3]: Rₜ=1.57 min; MS (ESIpos): m/z=406 [M+H]⁺.

¹H-NMR (400 MHz, DMSO-d₆): δ [ppm]=14.04 (br s, 1H), 12.17 (br s, 1H), 8.35 (s, 1H), 7.89 (s, 1H), 7.82-7.77 (m, 2H).

### Intermediate 301A

### Diethyl 1-[2-(5-methylquinolin-3-yl)-2-oxoethyl]-4-(trifluoromethyl)-1H-pyrazole-3,5-dicarboxylate

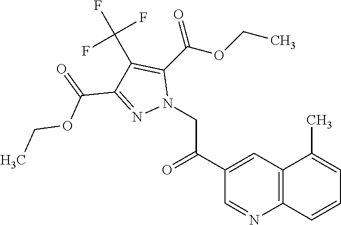 [SMILES: CCOC(=O)c1nn(CC(=O)c2cnc3cccc(C)c3c2)c(C(=O)OCC)c1C(F)(F)F]

Potassium carbonate (986 mg, 7.14 mmol) was added to a mixture of diethyl 4-(trifluoromethyl)-1H-pyrazole-3,5-dicarboxylate (Intermediate 32A, 800 mg, 2.86 mmol) and 2-bromo-1-(5-methylquinolin-3-yl)ethan-1-one (Intermediate 74A, 1.39 g, 65% purity, 3.43 mmol) in acetone (25 ml) and the mixture was stirred at RT overnight. Then, the mixture was filtered and the filter cake was washed with acetone. The combined filtrates were evaporated and dried under reduced pressure to afford the crude title compound which was used in the next step without further purification. Yield: 1.85 g (quant, 74% purity).

LC/MS [Method 7]: Rₜ=1.15 min; MS (ESIpos): m/z=464 [M+H]⁺.

### Intermediate 302A

### Ethyl 6-(5-methylquinolin-3-yl)-4-oxo-3-(trifluoromethyl)-4,5-dihydropyrazolo[1,5-a]pyrazine-2-carboxylate

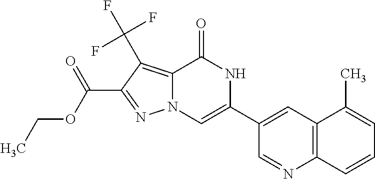 [SMILES: CCOC(=O)c1nn2cc(-c3cnc4cccc(C)c4c3)[nH]c(=O)c2c1C(F)(F)F]

A mixture of diethyl 1-[2-(5-methylquinolin-3-yl)-2-oxoethyl]-4-(trifluoromethyl)-1H-pyrazole-3,5-dicarboxylate (Intermediate 301A, 1.80 g, 74% purity, 2.87 mmol) and ammonium acetate (8.86 g, 115 mmol) in acetic acid (57 ml) was heated to 110° C. for three days. After cooling to RT, the mixture was poured into water (400 ml). The precipitate was collected by filtration, washed with ethyl acetate (10 ml) and MTBE (50 ml) and dried. The obtained solid was triturated with MTBE and dried to afford the title compound. Yield: 1.60 g (quant., 77% purity).

LC/MS [Method 3]: Rₜ=1.84 min; MS (ESIpos): m/z=417 [M+H]⁺.

### Intermediate 303A

### 6-(5-Methylquinolin-3-yl)-4-oxo-3-(trifluoromethyl)-4,5-dihydropyrazolo[1,5-a]pyrazine-2-carboxylic acid

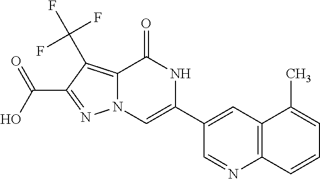 [SMILES: Cc1cccc2ncc(-c3cn4nc(C(=O)O)c(C(F)(F)F)c4c(=O)[nH]3)cc12]

A mixture of ethyl 6-(5-methylquinolin-3-yl)-4-oxo-3-(trifluoromethyl)-4,5-dihydropyrazolo[1,5-a]-pyrazine-2-carboxylate (Intermediate 302A, 1.60 g, 3.84 mmol) and lithium hydroxide (920 mg, 38.4 mmol) in water (7.7 ml) and ethanol (15 ml) was stirred at RT for 4 h. The ethanol was distilled off under reduced pressure and the residue was diluted with water and acidified by addition of 1.0 M hydrochloric acid to pH 2. The precipitate was collected by filtration and dried to afford the title compound. Yield: 1.06 g (61% of theory, 86% purity).

LC/MS [Method 3]: Rₜ=1.12 min; MS (ESIpos): m/z=389 [M+H]⁺.

### Intermediate 304A

### Diethyl 1-[2-(2,3-dihydro-1,4-benzodioxin-6-yl)-2-oxoethyl]-4-(trifluoromethyl)-1H-pyrazole-3,5-dicarboxylate

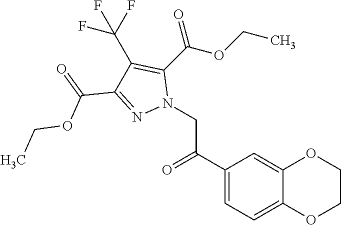 [SMILES: CCOC(=O)c1nn(CC(=O)c2ccc3c(c2)OCCO3)c(C(=O)OCC)c1C(F)(F)F]

To a solution of 2-bromo-1-(2,3-dihydro-1,4-benzodioxin-6-yl)ethan-1-one (Intermediate 99A, 3.56 g, 85% purity, 11.8 mmol) in acetone (80 ml) were added diethyl 4-(trifluoromethyl)-1H-pyrazole-3,5-dicarboxylate (Intermediate 32A, 3.00 g, 10.7 mmol) and potassium carbonate (3.69 g, 26.8 mmol). The reaction mixture was stirred at RT overnight. After filtering off the solids, the filtrate was concentrated under reduced pressure to provide the crude product. The crude product was purified by flash chromatography on silica gel (eluent: petroleum ether-ethyl acetate 4:1) to give the title compound. Yield: 4.50 g (98% purity, 90% of theory).

LC/MS [Method 5]: Rₜ=1.24 min; MS (ESIpos): m/z=479 [M+Na]⁺.

¹H-NMR (400 MHz, DMSO-d₆): δ [ppm]=7.58-7.55 (m, 2H), 7.07-7.04 (m, 1H), 6.18 (s, 2H), 4.39-4.22 (m, 8H), 1.30 (t, 3H), 1.14 (t, 3H).

### Intermediate 305A

### Ethyl 6-(2,3-dihydro-1,4-benzodioxin-6-yl)-4-oxo-3-(trifluoromethyl)-4,5-dihydropyrazolo[1,5-a]-pyrazine-2-carboxylate

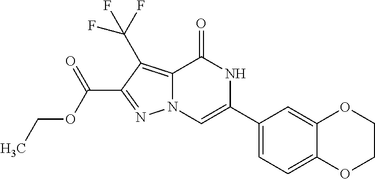 [SMILES: CCOC(=O)c1nn2cc(-c3ccc4c(c3)OCCO4)[nH]c(=O)c2c1C(F)(F)F]

To a solution of diethyl 1-[2-(2,3-dihydro-1,4-benzodioxin-6-yl)-2-oxoethyl]-4-(trifluoromethyl)-1H-pyrazole-3,5-dicarboxylate (Intermediate 304A, 4.50 g, 9.86 mmol) in acetic acid (60 ml) was added ammonium acetate (15.2 g, 197 mmol) and the mixture was heated to 110° C. overnight. After cooling to RT, the mixture was poured into ice-water (80 ml). The precipitate was collected by filtration, washed with water and dried to afford the title compound. Yield: 3.30 g (81% of theory).

LC/MS [Method 12]: Rₜ=1.11 min; MS (ESIpos): m/z=410 [M+H]⁺.

¹H-NMR (400 MHz, DMSO-d₆): δ [ppm]=8.17 (s, 1H), 7.33 (s, 1H), 7.28-7.25 (m, 1H), 6.98 (d, 1H), 4.39 (q, 2H), 4.31-4.28 (m, 4H), 1.33 (t, 3H).

### Intermediate 306A

### 6-(2,3-Dihydro-1,4-benzodioxin-6-yl)-4-oxo-3-(trifluoromethyl)-4,5-dihydropyrazolo[1,5-a]-pyrazine-2-carboxylic acid

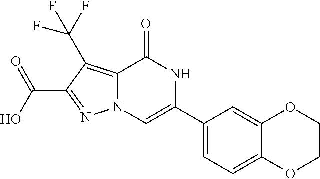 [SMILES: O=C(O)c1nn2cc(-c3ccc4c(c3)OCCO4)[nH]c(=O)c2c1C(F)(F)F]

To a solution of ethyl 6-(2,3-dihydro-1,4-benzodioxin-6-yl)-4-oxo-3-(trifluoromethyl)-4,5-dihydropyrazolo[1,5-a]pyrazine-2-carboxylate (Intermediate 305A, 3.30 g, 8.06 mmol) in ethanol (54 ml) and water (6.0 ml) was added sodium hydroxide (3.22 g, 80.6 mmol) and the resulting mixture was stirred at RT for 4 h. The solution was diluted with water (30 ml) and extracted with ethyl acetate (2×30 ml). Then, the aqueous layer was adjusted to pH =3 with 1N hydrochloric acid solution and the product precipitated. The solid was collected by filtration and dried to afford the title compound.

Yield: 2.43 g (77% of theory, 98% purity).

LC/MS [Method 24]: Rₜ=2.36 min; MS (ESIpos): m/z=382 [M+H]⁺.

¹H-NMR (400 MHz, DMSO-d₆): δ [ppm]=13.95 (brs, 1H), 11.95 (s, 1H), 8.11 (s, 1H), 7.32-7.24 (m, 2H), 6.97 (d, 1H), 4.30-4.25 (m, 4H).

### Intermediate 307A

### Diethyl 1-[2-(3,4-dichlorophenyl)-2-oxoethyl]-3-(trifluoromethyl)-1H-pyrrole-2,4-dicarboxylate

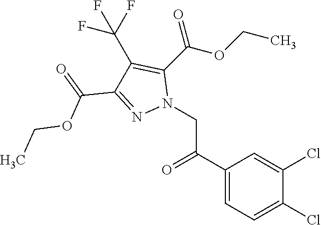 [SMILES: CCOC(=O)c1nn(CC(=O)c2ccc(Cl)c(Cl)c2)c(C(=O)OCC)c1C(F)(F)F]

To a solution of 2-bromo-1-(3,4-dichlorophenyl)ethanone (3.00 g, 11.20 mmol) in acetone (150 ml) were added diethyl 4-(trifluoromethyl)-1H-pyrazole-3,5-dicarboxylate (Intermediate 32A, 1.41 g, 5.60 mmol) and potassium carbonate (2.32 g, 16.80 mmol). The mixture was stirred for 2 hours at room temperature. After filtering off the solid, the filtrate was concentrated under reduced pressure. The crude product was triturated in ethyl acetate/petroleum ether (200 ml, 1:20). The solid was collected by filtration and dried to afford the title compound. Yield: 3.0 g (49% of theory, 84% purity).

¹H-NMR (400 MHz, DMSO-d₆): δ [ppm]=8.30 (d, 1H), 8.00 (d, 1H), 7.93 (d, 1H), 6.31 (s, 2H), 4.34 (q, 2H), 4.24 (q, 2H), 1.30 (t, 3H), 1.19 (t, 3H).

### Intermediate 308A

### Ethyl 6-(3,4-dichlorophenyl)-4-oxo-3-(trifluoromethyl)-4,5-dihydropyrazolo[1,5-a]pyrazine-2-carboxylate

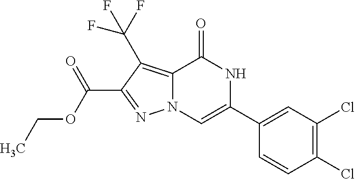 [SMILES: CCOC(=O)c1nn2cc(-c3ccc(Cl)c(Cl)c3)[nH]c(=O)c2c1C(F)(F)F]

To a solution of diethyl 1-[2-(3,4-dichlorophenyl)-2-oxoethyl]-4-(trifluoromethyl)-1H-pyrazole-3,5-dicarboxylate (Intermediate 307A, 3.00 g, 84% purity, 5.42 mmol) in acetic acid (50 ml) was added ammonium acetate (14.85 g, 162.58 mmol). The resulting mixture was heated to 110° C. overnight. After cooling to room temperature, the reaction mixture was poured into ice-water. The precipitate was collected by filtration, washed with water and dried in vacuo to give the title compound. Yield: 1.10 g (45% of theory, 93% purity).

LC/MS [Method 45]: Rₜ=1.48 min; MS (ESIpos): m/z=420 [M+H]⁺.

¹H-NMR (400 MHz, DMSO-d₆): δ [ppm]=12.21 (s, 1H), 8.41 (s, 1H), 8.09 (d, 1H), 7.83-7.75 (m, 2H), 4.40 (q, 2H), 1.33 (t, 3H).

### Intermediate 309A

### 6-(3,4-Dichlorophenyl)-4-oxo-3-(trifluoromethyl)-4,5-dihydropyrazolo[1,5-a]pyrazine-2-carboxylic acid

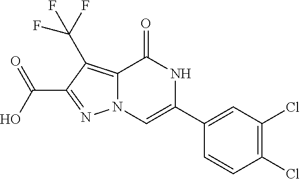 [SMILES: O=C(O)c1nn2cc(-c3ccc(Cl)c(Cl)c3)[nH]c(=O)c2c1C(F)(F)F]

To a solution of ethyl 6-(3,4-dichlorophenyl)-4-oxo-3-(trifluoromethyl)-4,5-dihydropyrazolo[1,5-a]-pyrazine-2-carboxylate (Intermediate 308A, 1.10 g, 93% purity, 2.45 mmol) in ethanol (50 ml) and water (13 ml) was added sodium hydroxide (2.09 g, 49.06 mmol). After stirring at room temperature for 2 hours, the reaction mixture was concentrated under reduced pressure. The residue was diluted with water and the pH value of the mixture was adjusted to 3-4 with hydrochloric acid. The formed precipitate was collected by filtration, washed with water and dried in air to give the title compound. Yield: 760 mg (65% of theory, 83% purity).

LC/MS [Method 41]: Rₜ=1.32 min; MS (ESIpos): m/z=414 [M+H]⁺.

¹H-NMR (400 MHz, DMSO-d₆): δ [ppm]=14.05 (s, 1H), 12.18 (s, 1H), 8.37 (s, 1H), 8.14-8.09 (m, 1H), 7.89-7.76 (m, 2H).

### Intermediate 310A

### Diethyl 4-cyclopropyl-1-{2-[4-methyl-3-(trifluoromethyl)phenyl]-2-oxoethyl}-1H-pyrazole-3,5-dicarboxylate

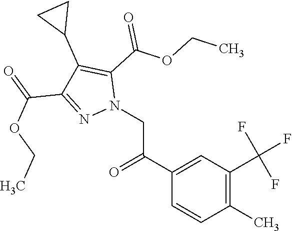 [SMILES: CCOC(=O)c1nn(CC(=O)c2ccc(C)c(C(F)(F)F)c2)c(C(=O)OCC)c1C1CC1]

To a solution of 2-bromo-1-[4-methyl-3-(trifluoromethyl)phenyl]ethan-1-one (Intermediate 194A, 3.70 g, 84% purity, 11.1 mmol) in acetone (150 ml) were added potassium carbonate (4.17 g, 30.2 mmol) and diethyl 4-cyclopropyl-1H-pyrazole-3,5-dicarboxylate (Intermediate 187A, 2.70 g, 94% purity, 10.1 mmol) and the mixture was stirred for 2 hours at room temperature. After filtering off the solids, the filtrate was concentrated under reduced pressure. The resulting crude product was suspended in ethyl acetate/petroleum ether (200 ml, 1:20) and stirred for 30 minutes. The solid was collected by filtration and dried to afford the title compound. Yield: 4.30 g (76% of theory, 81% purity).

LC/MS [Method 47]: Rₜ=2.39 min; MS (ESIpos): m/z=453 [M+H]⁺.

¹H-NMR (300 MHz, DMSO-d₆): δ [ppm]=8.25-8.22 (m, 2H), 7.71 (d, 1H), 6.20 (s, 2H), 4.34-4.27 (m, 2H), 4.24-4.16 (m, 2H), 2.57-2.56 (m, 3H), 2.10-2.02 (m, 1H), 1.33-1.29 (m, 3H), 1.19-1.13 (m, 3H), 0.97-0.90 (m, 2H),0.70-0.65 (m, 2H).

### Intermediate 311A

### Ethyl 3-cyclopropyl-6-[4-methyl-3-(trifluoromethyl)phenyl]-4-oxo-4,5-dihydropyrazolo[1,5-a]pyrazine-2-carboxylate

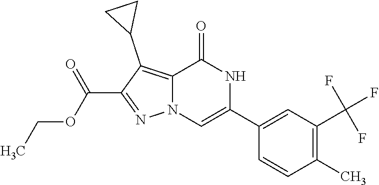 [SMILES: CCOC(=O)c1nn2cc(-c3ccc(C)c(C(F)(F)F)c3)[nH]c(=O)c2c1C1CC1]

To a solution of diethyl 4-cyclopropyl-1-{2-[4-methyl-3-(trifluoromethyl)phenyl]-2-oxoethyl}-1H-pyrazole-3,5-dicarboxylate (Intermediate 310A, 3.60 g, 82% purity, 6.51 mmol) in acetic acid (150 ml) was added ammonium acetate (15.5 g, 195 mmol). The resulting mixture was heated to 110° C. overnight. After cooling to room temperature, the reaction mixture was poured into ice-water. The precipitate was collected by filtration, washed with water and dried to afford the title compound. Yield: 2.74 g (93% of theory, 90% purity).

LC/MS [Method 46]: Rₜ=1.66 min; MS (ESIpos): m/z=406 [M+H]⁺.

¹H-NMR (300 MHz, DMSO-d₆): δ [ppm]=11.67 (s, 1H), 8.23 (s, 1H), 8.04 (d, 1H), 7.92 (dd, 1H), 7.56 (d, 1H), 4.32 (q, 2H), 2.49 (s, 3H), 2.71-2.64 (m, 1H), 1.33 (t, 3H), 1.28-1.21 (m, 2H), 0.99-0.92 (m, 2H).

### Intermediate 312A

### 3-Cyclopropyl-6-[4-methyl-3-(trifluoromethyl)phenyl]-4-oxo-4,5-dihydropyrazolo[1 ,5-a]pyrazine-2-carboxylic acid

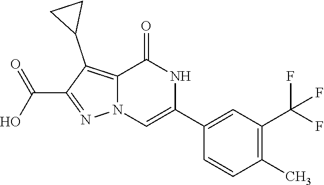 [SMILES: Cc1ccc(-c2cn3nc(C(=O)O)c(C4CC4)c3c(=O)[nH]2)cc1C(F)(F)F]

To a solution of ethyl 3-cyclopropyl-6-[4-methyl-3-(trifluoromethyl)phenyl]-4-oxo-4,5-dihydropyrazolo[1,5-a]pyrazine-2-carboxylate (Intermediate 311A, 2.74 g, 90% purity, 6.08 mmol) in ethanol (50 ml) was added sodium hydroxide solution (4.87 g, 122 mmol, dissolved in 20 ml of water). After stirring at room temperature for 2 hours, the reaction mixture was concentrated under reduced pressure. The residue was diluted with water and the pH value of the mixture was adjusted to 3˜4 with hydrochloric acid. The precipitate was collected by filtration, washed with water and dried to afford the title compound. Yield: 2.24 g (96% of theory, 98% purity).

LC/MS [Method 48]: Rₜ=0.93 min; MS (ESIpos): m/z=378 [M+H]⁺.

¹H-NMR (300 MHz, DMSO-d₆): δ [ppm]=13.06 (br s, 1H), 11.61 (s, 1H), 8.14 (s, 1H), 8.03 (s, 1H), 7.92 (d, 1H), 7.46 (d, 1H), 2.82-2.70 (m, 1H), 2.48 (s, 3H), 1.30-1.22 (m, 2H), 0.97-0.95 (m, 2H).

### Intermediate 313A

### Diethyl 4-cyclopropyl-1-[2-(2,3-dihydro-1,4-benzodioxin-6-yl)-2-oxoethyl]-1H-pyrazole-3,5-dicarboxylate

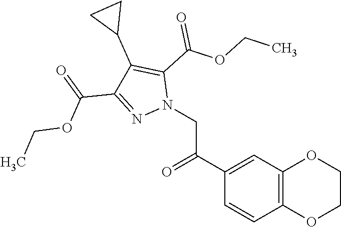 [SMILES: CCOC(=O)c1nn(CC(=O)c2ccc3c(c2)OCCO3)c(C(=O)OCC)c1C1CC1]

Potassium carbonate (2.24 g, 16.2 mmol) was added to a mixture of diethyl 4-cyclopropyl-1H-pyrazole-3,5-dicarboxylate (Intermediate 187A, 1.70 g, 96% purity, 6.48 mmol) and 2-bromo-1-(2,3-dihydro-1,4-benzodioxin-6-yl)ethan-1-one (Intermediate 99A, 2.00 g, 7.78 mmol) in acetone (70 ml) and the mixture was stirred at RT overnight. The mixture was filtered and the filter cake was washed with acetone. The combined filtrates were evaporated and dried under reduced pressure to afford the crude title compound which was used in the next step without further purification. Yield: 3.10 g (98% of theory, 88% purity).

LC/MS [Method 3]: Rₜ=2.09 min; MS (ESIpos): m/z=429 [M+H]⁺.

### Intermediate 314A

### Ethyl 3-cyclopropyl-6-(2,3-dihydro-1,4-benzodioxin-6-yl)-4-oxo-4,5-dihydropyrazolo[1,5-a]pyrazine-2-carboxylate

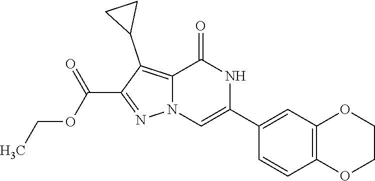 [SMILES: CCOC(=O)c1nn2cc(-c3ccc4c(c3)OCCO4)[nH]c(=O)c2c1C1CC1]

A mixture of diethyl 4-cyclopropyl-1-[2-(2,3-dihydro-1,4-benzodioxin-6-yl)-2-oxoethyl]-1H-pyrazole-3,5-dicarboxylate (Intermediate 313A, 3.10 g, 88% purity, 6.37 mmol) and ammonium acetate (12.3 g, 159 mmol) in acetic acid (60 ml) was heated to 110° C. for three days. After cooling to RT, the mixture was poured into water (700 ml). The precipitate was collected by filtration and dried to afford the title compound. Yield: 2.65 g (98% of theory, 90% purity).

LC/MS [Method 3]: Rₜ=1.85 min; MS (ESIpos): m/z=382 [M+H]⁺.

¹H-NMR (400 MHz, DMSO-d₆): δ [ppm]=11.37 (s, 1H), 7.98 (d, 1H), 7.29 (d, 1H), 7.23 (dd, 1H), 6.95 (d, 1H), 4.37-4.21 (m, 6H), 2.73-2.59 (m, 1H), 1.32 (t, 3H), 1.26-1.14 (m, 2H), 1.05-0.82 (m, 2H).

### Intermediate 315A

### 3-Cyclopropyl-6-(2,3-dihydro-1,4-benzodioxin-6-yl)-4-oxo-4,5-dihydropyrazolo[1,5-a]pyrazine-2-carboxylic acid

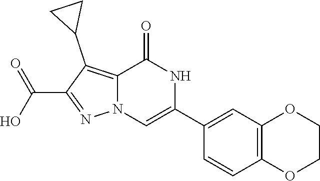 [SMILES: O=C(O)c1nn2cc(-c3ccc4c(c3)OCCO4)[nH]c(=O)c2c1C1CC1]

A mixture of ethyl 3-cyclopropyl-6-(2,3-dihydro-1,4-benzodioxin-6-yl)-4-oxo-4,5-dihydropyrazolo-[1,5-a]pyrazine-2-carboxylate (Intermediate 314A, 2.65 g, 90% purity, 6.25 mmol) and lithium hydroxide (749 mg, 31.3 mmol) in methanol (91 ml) and water (30 ml) was stirred at 50° C. for 3 h. More methanol (200 ml) and 1.0 M aqueous sodium hydroxide solution (10 ml) was added and stirring was continued at 50° C. overnight. The methanol was distilled off under reduced pressure and the residue was acidified by addition of 1.0 M hydrochloric acid. The precipitate was collected by filtration and dried to afford the title compound. Yield: 2.50 g (quant.).

LC/MS [Method 3]: Rₜ=1.32 min; MS (ESIpos): m/z=354 [M+H]⁺.

¹H-NMR (400 MHz, DMSO-d₆): δ [ppm]=13.06 (br s, 1H), 11.33 (s, 1H), 7.91 (d, 1H), 7.29 (d, 1H), 7.23 (dd, 1H), 6.95 (d, 1H), 4.32-4.20 (m, 4H), 2.77-2.67 (m, 1H), 1.32-1.17 (m, 2H), 0.98-0.80 (m, 2H).

### Intermediate 316A

### Diethyl 1-[2-(3-chloro-4-methylphenyl)-2-oxoethyl]-4-cyclopropyl-1H-pyrazole-3,5-dicarboxylate

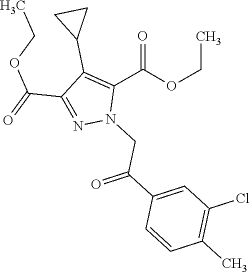 [SMILES: CCOC(=O)c1nn(CC(=O)c2ccc(C)c(Cl)c2)c(C(=O)OCC)c1C1CC1]

To a solution of 2-bromo-1-(3-chloro-4-methylphenyl)ethanone (19.75 g, 85% purity, 67.82 mmol) in acetone (400 ml) were added diethyl 4-cyclopropyl-1H-pyrazole-3,5-dicarboxylate (Intermediate 187A, 18.30 g, 85% purity, 61.66 mmol) and potassium carbonate (25.53 g, 184.97 mmol). After stirring for 2 h at room temperature, the solid was filtered off and the filtrate was evaporated under reduced pressure to give the title compound which was used in the next step without further purification. Yield: 29.00 g (96% of theory, 95% purity).

¹H-NMR (300 MHz, DMSO-d₆): δ [ppm]=8.06 (d, 1H), 7.91 (dd, 1H), 7.60 (d, 1H), 6.14 (s, 2H), 4.30 (q, 2H), 4.20 (q, 2H), 2.45 (s, 3H), 2.10-2.02 (m, 1H), 1.31 (t, 3H), 1.16 (t, 3H), 0.99-0.89 (m, 2H), 0.73-0.63 (m, 2H).

### Intermediate 317A

### Ethyl 6-(3-chloro-4-methylphenyl)-3-cyclopropyl-4-oxo-4,5-dihydropyrazolo[1,5-a]pyrazine-2-carboxylate

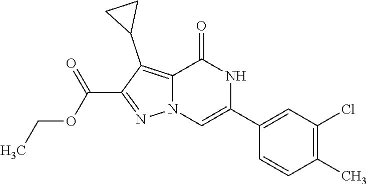 [SMILES: CCOC(=O)c1nn2cc(-c3ccc(C)c(Cl)c3)[nH]c(=O)c2c1C1CC1]

To a solution of diethyl 1-[2-(3-chloro-4-methylphenyl)-2-oxoethyl]-4-cyclopropyl-1H-pyrazole-3,5-dicarboxylate (Intermediate 316A, 27.00 g, 95% purity, 61.24 mmol) in acetic acid (400 ml) was added ammonium acetate (142 g, 1.84 mol). The resulting mixture was stirred at 100° C. overnight. After cooling to room temperature, the reaction mixture was poured into ice-water. The solid was collected by filtration, washed with water and dried in air to give the title compound. to Yield: 23.0 g (95% of theory, 95% purity).

¹H-NMR (400 MHz, DMSO-d₆): δ [ppm]=11.53 (s, 1H), 8.16 (s, 1H), 7.85 (s, 1H), 7.64 (dd, 1H), 7.47 (d, 1H), 4.34 (q, 2H), 2.76-2.60 (m, 1H), 2.38 (s, 3H), 1.34 (t, 3H), 1.28-1.15 (m, 2H), 1.08-0.82 (m, 2H).

### Intermediate 318A

### 6-(3-Chloro-4-methylphenyl)-3-cyclopropyl-4-oxo-4,5-dihydropyrazolo[1,5-a]pyrazine-2-carboxylic acid

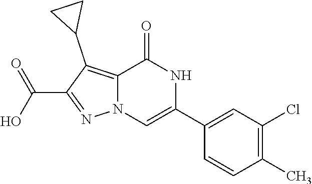 [SMILES: Cc1ccc(-c2cn3nc(C(=O)O)c(C4CC4)c3c(=O)[nH]2)cc1Cl]

To a solution of ethyl 6-(3-chloro-4-methylphenyl)-3-cyclopropyl-4-oxo-4,5-dihydropyrazolo-[1,5-a]pyrazine-2-carboxylate (Intermediate 317A, 23.00 g, 95% purity, 58.76 mmol) in ethanol (450 ml) and water (300 ml) was added sodium hydroxide (23.5 g, 588 mmol). After stirring overnight at 40° C., the reaction mixture was concentrated under reduced pressure. The residue was diluted with water (500 ml) and wash with MTBE. The pH value of the aqueous phase was adjusted to 3 with 2N hydrochloric acid. The precipitate was collected by filtration, washed with water and dried in air to give the title compound. Yield: 13.8 g (63% of theory, 93% purity).

¹H-NMR (300 MHz, DMSO-d₆): 6 [ppm]=13.13 (br s, 1H), 11.49 (s, 1H), 8.09 (s, 1H), 7.84 (d, 1H), 7.63 (dd, 1H), 7.46 (d, 1H), 2.90-2.54 (m, 1H), 2.38 (s, 3H), 1.34-1.17 (m, 2H), 1.02-0.84 (m, 2H).

### Intermediate 319A

### Diethyl 4-chloro-1-[2-(4-chloro-3-methylphenyl)-2-oxoethyl]-1H-pyrazole-3,5-dicarboxylate

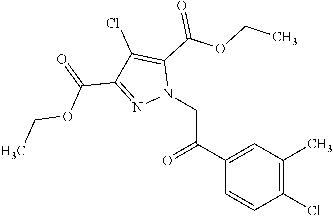 [SMILES: CCOC(=O)c1nn(CC(=O)c2ccc(Cl)c(C)c2)c(C(=O)OCC)c1Cl]

Potassium carbonate (2.02 g, 14.6 mmol) was added to a mixture of diethyl 4-chloro-1H-pyrazole-3,5-dicarboxylate (Intermediate 118A, 1.70 g, 85% purity, 5.86 mmol) and 2-bromo-1-(4-chloro-3-methylphenyl)ethan-1-one (1.60 g, 6.44 mmol) in acetone (53 ml) and the mixture was stirred at RT for three days. Then, the mixture was filtered and the filter cake was washed with acetone. The combined filtrates were evaporated and dried under reduced pressure to afford the crude title compound which was used in the next step without further purification. Yield: 2.32 g (80% of theory, 83% purity).

LC/MS [Method 3]: Rₜ=2.30 min; MS (ESIpos): m/z=413 [M+H]⁺.

### Intermediate 320A

### Ethyl 3-chloro-6-(4-chloro-3-methylphenyl)-4-oxo-4,5-dihydropyrazolo[1,5-a]pyrazine-2-carboxylate

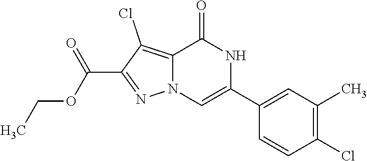 [SMILES: CCOC(=O)c1nn2cc(-c3ccc(Cl)c(C)c3)[nH]c(=O)c2c1Cl]

A mixture of diethyl 4-chloro-1-[2-(4-chloro-3-methylphenyl)-2-oxoethyl]-1H-pyrazole-3,5-dicarboxylate (Intermediate 319A, 2.32 g, 83% purity, 4.66 mmol) and ammonium acetate (14.4 g, 186 mmol) in acetic acid (88 ml) was heated to 110° C. for 6 d. More ammonium acetate (3.59 g, 46.5 mmol) was added and stirring was continued at 110° C. overnight. After cooling to room temperature, the reaction mixture was poured into water (600 ml). The solid was collected by filtration, washed with water and dried to give the title compound. Yield: 2.0 g (85% of theory, 70% purity).

LC/MS [Method 3]: Rₜ=1.93 min; MS (ESIpos): m/z=366 [M+H]⁺.

### Intermediate 321A

### 3-Chloro-6-(4-chloro-3-methylphenyl)-4-oxo-4,5-dihydropyrazolo[1,5-a]pyrazine-2-carboxylic acid

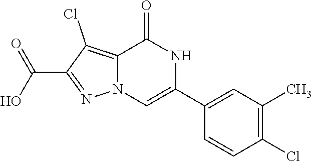 [SMILES: Cc1cc(-c2cn3nc(C(=O)O)c(Cl)c3c(=O)[nH]2)ccc1Cl]

A mixture of ethyl 3-chloro-6-(4-chloro-3-methylphenyl)-4-oxo-4,5-dihydropyrazolo[1,5-a]pyrazine-2-carboxylate (Intermediate 320A, 2.00 g, 70% purity, 3.82 mmol) and lithium hydroxide in ethanol (15 ml) and water (7.5 ml) was stirred at RT overnight. The ethanol was distilled off under reduced pressure and the residue was diluted with water and acidified by addition of 1.0 M hydrochloric acid.

The precipitate was collected by filtration and dried to afford the title compound. Yield: 769 mg (59% of theory).

LC/MS [Method 3]: Rₜ=1.44 min; MS (ESIpos): m/z=338 [M+H]⁺.

### Intermediate 322A

### Diethyl 4-chloro-1-[2-(3-chloro-4-methylphenyl)-2-oxoethyl]-1H-pyrazole-3,5-dicarboxylate

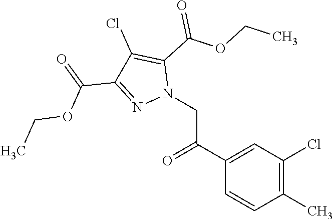 [SMILES: CCOC(=O)c1nn(CC(=O)c2ccc(C)c(Cl)c2)c(C(=O)OCC)c1Cl]

Potassium carbonate (3.15 g, 22.8 mmol) was added to a mixture of diethyl 4-chloro-1H-pyrazole-3,5-dicarboxylate (Intermediate 118A, 2.50 g, 90% purity, 9.12 mmol) and 2-bromo-1-(3-chloro-4-methylphenyl)ethan-1-one (3.55 g, 70% purity, 10.0 mmol) in acetone (82 ml) and the mixture was stirred at RT overnight. Then, the mixture was filtered and the filter cake was washed with acetone. The combined filtrates were evaporated and dried under reduced pressure to afford the crude title compound which was used in the next step without further purification. Yield: 4.01 g (74% of theory, 70% purity).

LC/MS [Method 3]: Rₜ=2.32 min; MS (ESIpos): m/z=413 [M+H]⁺.

### Intermediate 323A

### Ethyl 3-chloro-6-(3-chloro-4-methylphenyl)-4-oxo-4,5-dihydropyrazolo[1,5-a]pyrazine-2-carboxylate

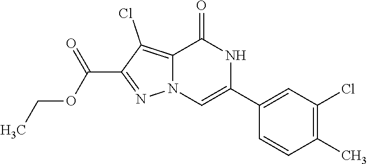 [SMILES: CCOC(=O)c1nn2cc(-c3ccc(C)c(Cl)c3)[nH]c(=O)c2c1Cl]

A mixture of diethyl 4-chloro-1-[2-(3-chloro-4-methylphenyl)-2-oxoethyl]-1H-pyrazole-3,5-dicarboxylate (Intermediate 322A, 4.09 g, 70% purity, 6.93 mmol) and ammonium acetate: (21.4 g, 277 mmol) in acetic acid (130 ml) was heated to 110° C. for three days. After cooling to room temperature, the reaction mixture was poured into water (600 ml). The solid was collected by filtration, washed with water and dried to give the title compound. Yield: 2.50 g (79% of theory, 80% purity).

LC/MS [Method 3]: Rₜ=1.93 min; MS (ESIpos): m/z=366 [M+H]⁺.

### Intermediate 324A

### 3-Chloro-6-(3-chloro-4-methylphenyl)-4-oxo-4,5-dihydropyrazolo[1,5-a]pyrazine-2-carboxylic acid

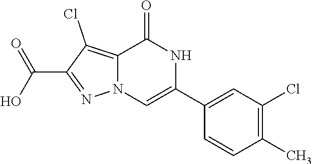 [SMILES: Cc1ccc(-c2cn3nc(C(=O)O)c(Cl)c3c(=O)[nH]2)cc1Cl]

A mixture of ethyl 3-chloro-6-(3-chloro-4-methylphenyl)-4-oxo-4,5-dihydropyrazolo[1,5-a]pyrazine-2-carboxylate (Intermediate 323A, 2.50 g, 6.83 mmol) and lithium hydroxide (817 mg, 34.1 mmol) in ethanol (27 ml) and water (13 ml) was stirred at RT overnight. The ethanol was distilled off under reduced pressure and the residue was diluted with water and acidified by addition of 1.0 M hydrochloric acid. The precipitate was collected by filtration and dried to afford the title compound. Yield: 1.13 g (45% of theory, 93% purity).

LC/MS [Method 3]: Rₜ=1.43 min; MS (ESIpos): m/z=338 [M+H]⁺.

¹H-NMR (600 MHz, DMSO-d₆): δ [ppm]=13.44 (br s, 1H), 11.79 (s, 1H), 8.18 (s, 1H), 7.84 (s, 1H), 7.64 (dd, 1H), 7.48 (d, 1H), 2.38 (s, 3H).

### Intermediate 325A

### Diethyl 1-[2-(4-chloro-3-methylphenyl)-2-oxoethyl]-4-(propan-2-yl)-1H-pyrazole-3,5-dicarboxylate

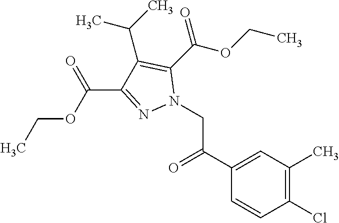 [SMILES: CCOC(=O)c1nn(CC(=O)c2ccc(Cl)c(C)c2)c(C(=O)OCC)c1C(C)C]

Potassium carbonate (1.36 g, 9.83 mmol) was added to a mixture of diethyl 4-(propan-2-yl)-1H-pyrazole-3,5-dicarboxylate (Intermediate 16A, 1.00 g, 3.93 mmol) and 2-bromo-1-(4-chloro-3-methylphenyl)ethan-1-one (1.44 g, 81% purity, 4.72 mmol) in acetone (40 ml) and the mixture was stirred at RT overnight. Then, the mixture was filtered and filtrate was evaporated and dried under reduced pressure to afford the crude title compound which was used in the next step without further purification. Yield: 1.80 g (99% of theory, 91% purity).

LC/MS [Method 3]: Rₜ=2.48 min; MS (ESIpos): m/z=421 [M+H]⁺.

### Intermediate 326A

### Ethyl 6-(4-chloro-3-methylphenyl)-4-oxo-3-(propan-2-yl)-4,5-dihydropyrazolo[1,5-a]pyrazine-2-carboxylate

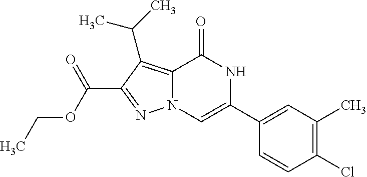 [SMILES: CCOC(=O)c1nn2cc(-c3ccc(Cl)c(C)c3)[nH]c(=O)c2c1C(C)C]

A mixture of diethyl 1-[2-(4-chloro-3-methylphenyl)-2-oxoethyl]-4-(propan-2-yl)-1H-pyrazole-3,5-dicarboxylate (Intermediate 325A, 1.80 g, 91% purity, 3.89 mmol) and ammonium acetate (12.0 g, 156 mmol) in acetic acid (70 ml) was heated to 110° C. overnight. After cooling to room temperature, the reaction mixture was poured into water (100 ml). The solid was collected by filtration and dried to give the title compound. Yield: 1.55 g (99% of theory, 93% purity).

LC/MS [Method 3]: Rₜ=2.27 min; MS (ESIpos): m/z=374 [M+H]⁺.

¹H-NMR (400 MHz, DMSO-d₆): δ [ppm]=11.56 (s, 1H), 8.14 (s, 1H), 7.79 (d, 1H), 7.60 (dd, 1H), 7.53 (d, 1H), 4.34 (q, 2H), 4.18-4.07 (m, 1H), 2.39 (s, 3H), 1.36 (d, 6H), 1.33 (t, 3H).

### Intermediate 327A

### 6-(4-Chloro-3-methylphenyl)-4-oxo-3-(propan-2-yl)-4,5-dihydropyrazolo[1,5-a]pyrazine-2-carboxylic acid

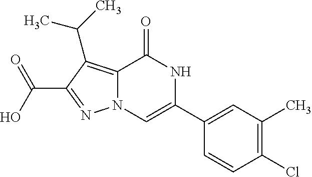 [SMILES: Cc1cc(-c2cn3nc(C(=O)O)c(C(C)C)c3c(=O)[nH]2)ccc1Cl]

A mixture of ethyl 6-(4-chloro-3-methylphenyl)-4-oxo-3-(propan-2-yl)-4,5-dihydropyrazolo[1,5-a]-pyrazine-2-carboxylate (Intermediate 326A, 1.55 g, 93% purity, 3.86 mmol) and lithium hydroxide in ethanol (39 ml) and water (20 ml) was stirred at RT for three days. The ethanol was distilled off under reduced pressure and the residue was diluted with water and acidified by addition of 1.0 M hydrochloric acid. The precipitate was collected by filtration and dried to afford the title compound. Yield: 1.45 g (98% of theory, 90% purity).

LC/MS [Method 3]: Rₜ=1.75 min; MS (ESIneg): m/z=344 [M−H]⁻.

¹H-NMR (400 MHz, DMSO-d₆): δ [ppm]=13.00 (br s, 1H), 11.50 (br s, 1H), 8.06 (s, 1H), 7.79 (d, 1H), 7.60 (dd, 1H), 7.52 (d, 1H), 4.12 (spt, 1H), 2.39 (s, 3H), 1.36 (d, 6H).

### Intermediate 328A

### 2-Bromo-1-(4-chloro-3,5-difluorophenyl)ethan-1-one

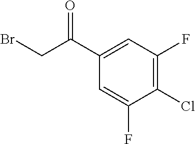 [SMILES: O=C(CBr)c1cc(F)c(Cl)c(F)c1]

Trimethylphenylammonium tribromide (10.4 g, 27.5 mmol) was added portion wise to a solution of 1-(4-chloro-3,5-difluorophenyl)ethan-1-one (5.25 g, 27.5 mmol) in THF (53 ml). The reaction mixture was stirred at RT overnight, the insoluble material was removed by filtration and the filtrate was concentrated under reduced pressure to afford the title compound which was used in the next step without further purification. Yield: 10.3 g (quant., 75% purity).

GC/MS [Method 35]: Rₜ=4.62 min; MS (ESIneg): m/z=269 [M+H]⁺.

### Intermediate 329A

### Diethyl 1-[2-(4-chloro-3,5-difluorophenyl)-2-oxoethyl]-4-methyl-1H-pyrazole-3,5-dicarboxylate

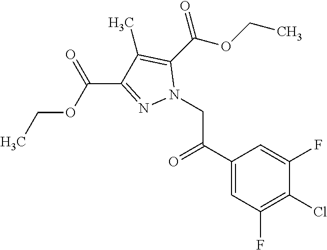 [SMILES: CCOC(=O)c1nn(CC(=O)c2cc(F)c(Cl)c(F)c2)c(C(=O)OCC)c1C]

Potassium carbonate (4.33 g, 31.3 mmol) was added to a mixture of diethyl 4-methyl-1H-pyrazole-3,5-dicarboxylate (Intermediate 54A, 2.83 g, 12.5 mmol) and 2-bromo-1-(4-chloro-3,5-difluorophenyl)ethan-1-one (Intermediate 328A, 5.40 g, 75% purity, 15.0 mmol) in acetone (110 ml) and the mixture was stirred at RT overnight. Then, the mixture was filtered and the filter cake was washed with acetone. The combined filtrates were evaporated and dried under reduced pressure to afford the crude title compound which was used in the next step without further purification. Yield: 6.67 g (91% of theory, 71% purity).

LC/MS [Method 11]: Rₜ=3.98 min; MS (ESIpos): m/z=415 [M+H]⁺.

### Intermediate 330A

### Ethyl 6-(4-chloro-3,5-difluorophenyl)-3-methyl-4-oxo-4,5-dihydropyrazolo[1,5-a]pyrazine-2-carboxylate

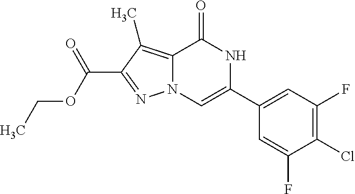 [SMILES: CCOC(=O)c1nn2cc(-c3cc(F)c(Cl)c(F)c3)[nH]c(=O)c2c1C]

A mixture of diethyl 1-[2-(4-chloro-3,5-difluorophenyl)-2-oxoethyl]-4-methyl-1H-pyrazole-3,5-dicarboxylate (Intermediate 329A, 6.67 g, 16.1 mmol) and ammonium acetate (49.6 g, 643 mmol) in acetic acid (260 ml) was heated to 110° C. for three days. After cooling to room temperature, the reaction mixture was poured into water (400 ml). The solid was collected by filtration, washed with ethyl acetate (10 ml) and MTBE (50 ml) and dried to give the title compound. Yield: 3.47 g (59% of theory).

LC/MS [Method 3]: Rₜ=1.92 min; MS (ESIneg): m/z=366 [M−H]⁻.

¹H-NMR (400 MHz, DMSO-d₆): δ [ppm]=11.61 (br s, 1H), 8.35 (s, 1H), 7.82 (d, 2H), 4.34 (q, 2H), 2.64 (s, 3H), 1.34 (t, 3H).

### Intermediate 331A

### 6-(4-Chloro-3,5-difluorophenyl)-3-methyl-4-oxo-4,5-dihydropyrazolo[1,5-a]pyrazine-2-carboxylic acid

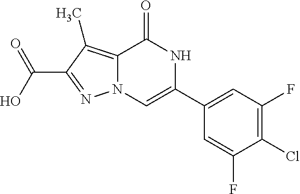 [SMILES: Cc1c(C(=O)O)nn2cc(-c3cc(F)c(Cl)c(F)c3)[nH]c(=O)c12]

A mixture of ethyl 6-(4-chloro-3,5-difluorophenyl)-3-methyl-4-oxo-4,5-dihydropyrazolo[1,5-a]pyrazine-2-carboxylate (Intermediate 330A, 3.47 g, 9.44 mmol) and lithium hydroxide (2.26 g, 94.4 mmol) in ethanol (38 ml) and water (19 ml) was stirred at RT for 4 h. The ethanol was distilled off under reduced pressure and the residue was diluted with water and acidified by addition of 1.0 M hydrochloric acid. The precipitate was collected by filtration and dried to afford the title compound. Yield: 3.55 g (quant.).

LC/MS [Method 3]: Rₜ=1.44 min; MS (ESIpos): m/z=340 [M+H]⁺.

¹H-NMR (400 MHz, DMSO-d₆): δ [ppm]=13.16 (br s, 1H), 11.53 (br s, 1H), 8.28 (s, 1H), 7.86-7.80 (m, 2H), 2.62 (s, 3H).

### Intermediate 332A

### 2-Bromo-1-(3-bromo-4-methylphenyl)ethanone

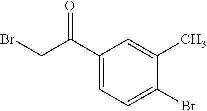

A solution of 1-(4-bromo-3-methylphenyl)ethanone (10.0 g, 46.9 mmol) and phenyltrimethylammonium tribromide (19.5 g, 51.6 mmol) in dichloromethane (300 ml) was stirred overnight at room temperature. The ammonium salts were filtered off and the filter cake was washed with dichloromethane. The filtrate was evaporated under reduced pressure to give 15.00 g (94% of theory, 84% purity) of the title compound.

LC/MS [Method 49]: Rₜ=1.54 min; MS (ESIpos): m/z=291 [M+H]⁺.

¹H-NMR (400 MHz, DMSO-d₆): δ [ppm]=8.06-7.64 (m, 3H), 4.93 (s, 2H), 2.40-2.45 (m, 3H).

### Intermediate 333A

### Diethyl 1-[2-(4-bromo-3-methylphenyl)-2-oxoethyl]-1H-pyrazole-3,5-dicarboxylate

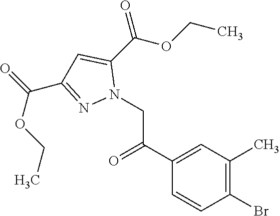 [SMILES: CCOC(=O)c1cc(C(=O)OCC)n(CC(=O)c2ccc(Br)c(C)c2)n1]

To a solution of diethyl 1H-pyrazole-3,5-dicarboxylate (8.52 g, 40.2 mmol) in acetone (150 ml) were added 2-bromo-1-(4-bromo-3-methylphenyl)ethanone (Intermediate 332A, 15.0 g, 86% purity, 44.2 mmol) and potassium carbonate 8.31 g (60.3 mmol). The mixture was stirred for 2 hours at room temperature. After filtering off the solids, the filtrate was concentrated under reduced pressure. The residue was purified by column chromatography (eluent: ethyl acetate/petroleum ether 9:1) to afford 12.0 g (58% of theory, 83% purity) of the title compound.

¹H-NMR (400 MHz, DMSO-d₆): δ [ppm]=8.05 (d, 1H), 7.84-7.78 (m, 2H), 7.36 (s, 1H), 6.24 (s, 2H), 4.32 (q, 2H), 4.21 (q, 2H), 2.47 (s, 3H), 1.31 (t, 3H), 1.20 (t, 3H).

### Intermediate 334A

### Ethyl 6-(4-bromo-3-methylphenyl)-4-oxo-4,5-dihydropyrazolo[1,5-a]pyrazine-2-carboxylate

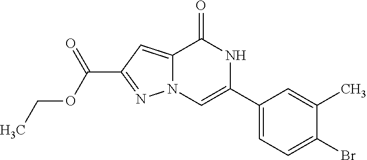 [SMILES: CCOC(=O)c1cc2c(=O)[nH]c(-c3ccc(Br)c(C)c3)cn2n1]

To a solution of diethyl 1-[2-(4-bromo-3-methylphenyl)-2-oxoethyl]-1H-pyrazole-3,5-dicarboxylate (Intermediate 333A, 12.0 g, 23.5 mmol, 83% purity) in acetic acid (300 ml) was added ammonium acetate (54.4 g, 0.706 mol). The resulting mixture was heated overnight at 110° C. After cooling to room temperature, the reaction mixture was poured into ice-water. The solid was collected by filtration, washed with water and dried in air to give 7.77 g (92% of theory, 93% purity) of the title compound.

LC/MS [Method 45]: Rₜ=1.38 min; MS (ESIpos): m/z=376 [M+H]⁺.

¹H-NMR (400 MHz, DMSO-d₆): δ [ppm]=11.79 (s, 1H), 8.22 (d, 1H), 7.78 (d, 1H), 7.70 (d, 1H), 7.53-7.43 (m, 1H), 7.41 (s, 1H), 4.35 (q, 2H), 2.48 (s, 3H), 1.34 (d, 3H).

### Intermediate 335A

### Ethyl 6-(4-cyclopropyl-3-methylphenyl)-4-oxo-4,5-dihydropyrazolo[1,5-a]pyrazine-2-carboxylate

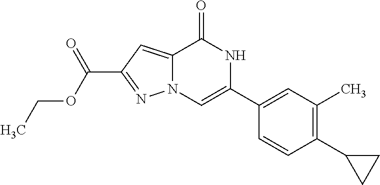 [SMILES: CCOC(=O)c1cc2c(=O)[nH]c(-c3ccc(C4CC4)c(C)c3)cn2n1]

To a solution of ethyl 6-(4-bromo-3-methylphenyl)-4-oxo-4,5-dihydropyrazolo[1,5-a]pyrazine-2-carboxylate (Intermediate 334A, 500 mg, 1.33 mmol) in THF (24 ml) was added [(2-dicyclohexylphosphino-2′,6′-bis(N,N-dimethylamino)-1,1′-biphenyl)-2-(2′-amino-1,1-biphenyl)] palladium(II) methanesulfonate (CPhos-Pd-G3, 214 mg, 266 μmol) under argon. At 0° C., bromido(cyclopropyl)zinc (20 ml, 0.50 M solution in THF, 10 mmol) was added dropwise. After complete addition, the cooling bath was removed and the mixture was allowed to stir at RT for 3 days. The mixture was directly purified by preparative HPLC (Method P14) to afford the title compound. Yield: 160 mg (27% of theory, 75% purity).

LC/MS [Method 3]: Rₜ=1.92 min; MS (ESIpos): m/z=338 [M+H]⁺.

### Intermediate 336A

### 6-(4-Cyclopropyl-3-methylphenyl)-4-oxo-4,5-dihydropyrazolo[1,5-a]pyrazine-2-carboxylic acid

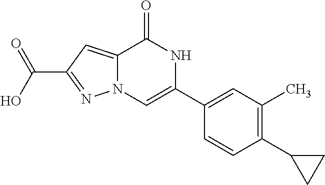 [SMILES: Cc1cc(-c2cn3nc(C(=O)O)cc3c(=O)[nH]2)ccc1C1CC1]

A mixture of ethyl 6-(4-cyclopropyl-3-methylphenyl)-4-oxo-4,5-dihydropyrazolo[1,5-a]pyrazine-2-carboxylate (Intermediate 335A, 160 mg, 75% purity, 356 μmol) and lithium hydroxide (85.2 mg, 3.56 mmol) in ethanol (3.0 ml) and water (1.0 ml) was stirred at RT for overnight. The reaction mixture was brought to pH 5 by addition of hydrochloric acid and the solution was directly purified by preparative HPLC (Method P14) to afford the title compound. Yield: 87.4 mg (79% of theory.).

LC/MS [Method 3]: Rₜ=1.47 min; MS (ESIpos): m/z=310 [M+H]⁺.

¹H-NMR (400 MHz, DMSO-d₆): δ [ppm]=13.21 (br s, 1H), 11.65 (s, 1H), 8.08 (s, 1H), 7.58 (s, 1H), 7.49 (d, 1H), 7.34 (s, 1H), 7.02 (d, 1H), 2.44 (s, 3H), 2.00-1.92 (m, 1H), 1.00-0.92 (m, 2H), 0.70-0.62 (m, 2H).

### Intermediate 337A

### 2-Bromo-1-(3-bromo-4-methylphenyl)ethan-1-one

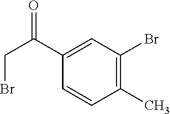

A solution of 1-(3-bromo-4-methylphenyl)ethanone (9.00 g, 42.2 mmol) and phenyltrimethylammonium tribromide (17.00 g, 46.5 mmol) in dichloromethane (200 ml) was stirred overnight at room temperature. The ammonium salts were filtered off and the filter cake was washed with ethyl acetate. The filtrate was evaporated under reduced pressure to give 13.00 g (85% of theory, 81% purity) of the title compound, which was used for next step directly without further purification.

LC/MS [Method 49]: Rₜ=1.51 min; MS (ESIpos): m/z=291 [M+H]⁺.

¹H-NMR (400 MHz, DMSO-d₆): δ [ppm]=8.26 (d, 1H), 7.93-7.86 (m, 1H), 7.57-7.51 (m, 1H), 4.95 (s, 2H), 2.46-2.42 (m, 3H).

### Intermediate 338A

### Diethyl 1-[2-(3-bromo-4-methylphenyl)-2-oxoethyl]-1H-pyrazole-3,5-dicarboxylate

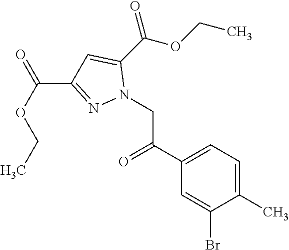 [SMILES: CCOC(=O)c1cc(C(=O)OCC)n(CC(=O)c2ccc(C)c(Br)c2)n1]

To a solution of diethyl 1H-pyrazole-3,5-dicarboxylate (7.07 g, 33.3 mmol) in acetone (120 ml) were added 2-bromo-1-(3-bromo-4-methylphenyl)ethanone (Intermediate 337A, 13.2 g (81% purity, 36.6 mmol) and potassium carbonate (6.89 g, 49.9 mmol). The mixture was stirred at RT for 2 h. After filtering off the solids, the filtrate was concentrated under reduced pressure to provide the crude product. The crude product was suspended in ethyl acetate/petroleum ether (300 ml, 1:20) and stirred for 30 minutes. The solid was collected by filtration and dried to give the title compound. Yield: 10.7 g (75% of theory, 99% purity).

¹H-NMR (400 MHz, DMSO-d₆): δ [ppm]=8.20 (d, 1H), 7.95 (dd, 1H), 7.58 (d, 1H), 7.34 (s, 1H), 6.23 (s, 2H), 4.29 (q, 2H), 4.18 (q, 2H), 2.44 (s, 3H), 1.29 (t, 3H), 1.17 (t, 3H).

### Intermediate 339A

### Ethyl 6-(3-bromo-4-methylphenyl)-4-oxo-4,5-dihydropyrazolo[1,5-a]pyrazine-2-carboxylate

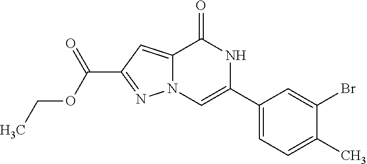 [SMILES: CCOC(=O)c1cc2c(=O)[nH]c(-c3ccc(C)c(Br)c3)cn2n1]

To a solution of diethyl 1-[2-(3-bromo-4-methylphenyl)-2-oxoethyl]-1H-pyrazole-3,5-dicarboxylate (Intermediate 338A, 10.7 g, 25.0 mmol, 99% purity) in acetic acid (350 ml) was added ammonium acetate (57.81 g, 751 mmol).The resulting mixture was heated to 110° C. overnight. After cooling to room temperature, the reaction mixture was poured into ice-water. The solid was collected by filtration, washed with water and dried in vacuo to give the title compound. Yield: 6.20 g (64% of theory, 98% purity)

LC/MS [Method 49]: Rₜ=1.37 min; MS (ESIpos): m/z=376 [M+H]⁺.

¹H-NMR (300 MHz, DMSO-d₆): δ [ppm]=11.82 (s, 1H), 8.26 (s, 1H), 8.01 (d, 1H), 7.67 (d, 1H), 7.48 (d, 1H), 7.41 (d, 1H), 4.35 (q, 2H), 2.40 (s, 3H), 1.34 (t, 3H).

### Intermediate 340A

### Ethyl 6-(3-cyclopropyl-4-methylphenyl)-4-oxo-4,5-dihydropyrazolo[1,5-a]pyrazine-2-carboxylate

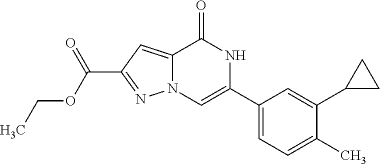 [SMILES: CCOC(=O)c1cc2c(=O)[nH]c(-c3ccc(C)c(C4CC4)c3)cn2n1]

To a solution of ethyl ethyl 6-(3-bromo-4-methylphenyl)-4-oxo-4,5-dihydropyrazolo[1,5-a]-pyrazine-2-carboxylate (Intermediate 339A, 500 mg, 1.33 mmol) in THF (24 ml) was added [(2-dicyclohexylphosphino-2′,6′-bis(N,N-dimethylamino)-1,1′-biphenyl)-2-(2′-amino-1,1-biphenyl)] palladium(II) methanesulfonate (CPhos-Pd-G3, 214 mg, 266 μmol) under argon. At 0° C., bromido(cyclopropyl)zinc (20 ml, 0.50 M solution in THF, 10 mmol) was added dropwise. After complete addition, the cooling bath was removed and the mixture was allowed to stir at RT for 3 days. The mixture was directly purified by preparative HPLC (Method P14) to afford the title compound. Yield: 160 mg (27% of theory, 75% purity).

LC/MS [Method 27]: Rₜ=1.32 min; MS (ESIneg): m/z=336 [M−H]⁻.

### Intermediate 341A

### 6-(3-Cyclopropyl-4-methylphenyl)-4-oxo-4,5-dihydropyrazolo[1,5-a]pyrazine-2-carboxylic acid

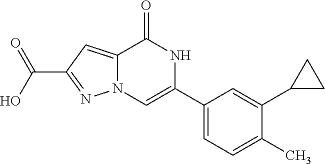 [SMILES: Cc1ccc(-c2cn3nc(C(=O)O)cc3c(=O)[nH]2)cc1C1CC1]

A mixture of ethyl 6-(3-cyclopropyl-4-methylphenyl)-4-oxo-4,5-dihydropyrazolo[1,5-a]pyrazine-2-carboxylate (Intermediate 340A, 330 mg, 978 μmol) and lithium hydroxide (234 mg, 9.78 mmol) in methanol (4.0 ml) and water (2.0 ml) was stirred at RT for overnight. The methanol was distilled off under reduced pressure and the residue was diluted with water and acidified by addition of 1.0 M hydrochloric acid. The precipitate was collected by filtration and dried to afford the title compound. Yield: 340 mg (quant., 88% purity).

LC/MS [Method 3]: Rₜ=1.45 min; MS (ESIpos): m/z=310 [M+H]⁺.

¹H-NMR (400 MHz, DMSO-d₆): δ [ppm]=11.67 (br s, 1H), 8.11 (s, 1H), 7.48-7.44 (m, 1H), 7.32-7.23 (m, 3H), 2.42 (s, 3H), 1.99-1.91 (m, 1H), 0.96-0.91 (m, 2H), 0.85-0.77 (m, 2H).

### Intermediate 342A

### Diethyl 4-propyl-1H-pyrazole-3,5-dicarboxylate

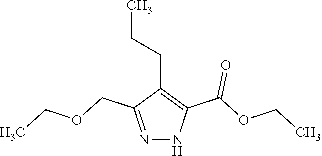 [SMILES: CCCc1c(COCC)n[nH]c1C(=O)OCC]

To a solution of ethyl 3-oxohexanoate (3.00 g, 19.0 mmol) in toluene (120 ml) was added a solution of lithium hydroxide (3.41 g, 142 mmol) in water (35 ml) at 10° C. The mixture was stirred for 5 min before trifluoromethanesulfonic anhydride (6.4 ml, 38 mmol) was added dropwise while the temperature was kept below 25° C. After stirring at RT for 2 h, the mixture was poured into water (150 ml) and extracted with ethyl acetate. The combined organic layers were washed with water and brine, dried over sodium sulfate and concentrated under reduced pressure to leave crude ethyl-3-[(trifluoromethanesulfonyl)oxy]hex-2-enoate (6.00 g, 20.7 mmol). The crude ethyl-3-[(trifluoromethanesulfonyl)oxy]hex-2-enoate (6.00 g, 20.7 mmol) was taken up in DMF (61 ml). Ethyl diazoacetate (3.54 g, 31.0 mmol) and tetrakis(triphenylphosphin)palladium(0) (1.19 g, 1.03 mmol) were added and the mixture was degassed for 5 min using a stream of argon. Then, N-methylmorpholine (4.5 ml, 41 mmol) was added dropwise and the mixture was stirred at RT for 3 h and overnight at 60° C. After cooling to RT, the reaction mixture was directly purified by preparative HPLC (method P14) to afford the title compound. Yield: 3.00 g (57% of theory).

LC/MS [Method 3]: Rₜ=1.76 min; MS (ESIneg): m/z=253 [M−H]⁻.

¹H-NMR (500 MHz, DMSO-d₆): δ [ppm]=14.31 (br s, 1H), 4.37-4.24 (m, 4H), 2.93 (t, 2H), 1.55-1.46 (m, 2H), 1.36-1.27 (m, 6H), 0.87 (t, 3H).

### Intermediate 343A

### Diethyl 1-[2-(3,4-dimethylphenyl)-2-oxoethyl]-4-propyl-1H-pyrazole-3,5-dicarboxylate

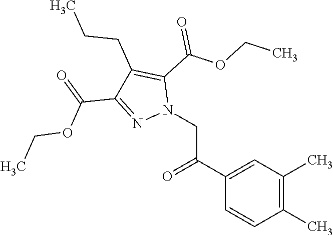 [SMILES: CCCc1c(C(=O)OCC)nn(CC(=O)c2ccc(C)c(C)c2)c1C(=O)OCC]

A mixture of 2-bromo-1-(3,4-dimethylphenyl)ethan-1-one (Intermediate 20A, 1.53 g, 70% purity, 4.72 mmol), diethyl 4-propyl-1H-pyrazole-3,5-dicarboxylate (Intermediate 342A, 1.00 g, 3.93 mmol) and potassium carbonate (1.36 g, 9.83 mmol) in acetone (35 ml) was stirred at RT overnight. After filtering off the solids, the filtrate was concentrated under reduced pressure to provide the crude product which was used in the next step without further purification. Yield: 2.20 g (quant., 72% purity).

LC/MS [Method 3]: Rₜ=2.41 min; MS (ESIpos): m/z=401 [M+H]⁺.

### Intermediate 344A

### Ethyl 6-(3,4-dimethylphenyl)-4-oxo-3-propyl-4,5-dihydropyrazolo[1,5-a]pyrazine-2-carboxylate

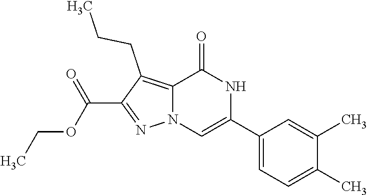 [SMILES: CCCc1c(C(=O)OCC)nn2cc(-c3ccc(C)c(C)c3)[nH]c(=O)c12]

A mixture of diethyl 1-[2-(3,4-dimethylphenyl)-2-oxoethyl]-4-propyl-1H-pyrazole-3,5-dicarboxylate (Intermediate 343A, 2.20 g, 72% purity, 3.96 mmol) and ammonium acetate (12.2 g, 158 mmol) in acetic acid (80 ml) was heated to 110° C. for four days. After cooling to RT, the mixture was poured into water (300 ml) and the precipitate was collected by filtration and dried to afford the crude title compound which was used in the next step without further purification. Yield: 1.70 g (97% of theory, 80% purity).

LC/MS [Method 3]: Rₜ=2.19 min; MS (ESIneg): m/z=352 [M−H]⁻.

¹H-NMR (500 MHz, DMSO-d₆): δ [ppm]=11.45 (s, 1H), 8.02 (s, 1H), 7.56 (s, 1H), 7.47 (d, 1H), 7.24 (d, 1H), 4.33 (q, 2H), 3.13 (t, 2H), 2.28 (s, 3H), 2.27 (s, 3H), 1.65-1.56 (m, 2H), 1.33 (t, 3H), 0.90 (t, 3H).

### Intermediate 345A

### 6-(3,4-Dimethylphenyl)-4-oxo-3-propyl-4,5-dihydropyrazolo[1,5-a]pyrazine-2-carboxylic acid

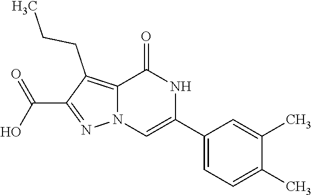 [SMILES: CCCc1c(C(=O)O)nn2cc(-c3ccc(C)c(C)c3)[nH]c(=O)c12]

A mixture of ethyl 6-(3,4-dimethylphenyl)-4-oxo-3-propyl-4,5-dihydropyrazolo[1,5-a]pyrazine-2-carboxylate (Intermediate 344A, 1.70 g, 80% purity, 3.85 mmol) and lithium hydroxide (461 mg, 19.2 mmol) in methanol (40 ml) and water (10 ml) was stirred at 50° C. overnight. After cooling to RT, the methanol was distilled off under reduced pressure and the residue was diluted with water and acidified by addition of 1.0 M hydrochloric acid. The precipitate was collected by filtration and dried to afford the title compound. Yield: 1.14 g (81% of theory, 89% purity).

LC/MS [Method 3]: Rₜ=1.66 min; MS (ESIpos): m/z=326 [M+H]⁺.

¹H-NMR (500 MHz, DMSO-d₆): δ [ppm]=13.05 (br s, 1H), 11.41 (s, 1H), 7.96 (s, 1H), 7.56 (s, 1H), 7.47 (dd, 1H), 7.24 (d, 1H), 3.17-3.09 (m, 2H), 2.28 (s, 3H), 2.27 (s, 3H), 1.65-1.55 (m, 2H), 0.89 (t, 3H).

### Intermediate 346A

### Diethyl 1-[2-(2,3-dihydro-1,4-benzodioxin-6-yl)-2-oxoethyl]-4-iodo-1H-pyrazole-3,5-dicarboxylate

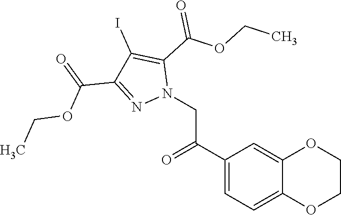 [SMILES: CCOC(=O)c1nn(CC(=O)c2ccc3c(c2)OCCO3)c(C(=O)OCC)c1I]

To a solution of 2-bromo-1-(2,3-dihydro-1,4-benzodioxin-6-yl)ethan-1-one (Intermediate 99A, 5.40 g, 82% purity, 17.2 mmol) in acetone (150 ml) were added diethyl 4-iodo-1H-pyrazole-3,5-dicarboxylate (Intermediate 78A, 5.29 g, 15.7 mmol) and potassium carbonate (5.41 g, 39.2 mmol). After stirring at room temperature overnight, the solids were filtered off and the filtrate was concentrated under reduced pressure. The residue was purified by flash chromatography on silica gel (240 g silica gel, eluent: petroleum ether-ethyl acetate 3:1) to give the title compound. Yield: 7.00 g (85% of theory, 98% purity).

LC/MS [Method 18]: Rₜ=1.06 min; MS (ESIpos): m/z=515 [M+H]⁺.

¹H-NMR (300 MHz, DMSO-d₆): δ [ppm]=7.58-7.54 (m, 2H), 7.05 (d, 1H), 6.17 (s, 2H), 4.40-4.28 (m, 6H), 4.21 (q, 2H), 1.31 (t, 3H), 1.13 (t, 3H).

### Intermediate 347A

### Ethyl 6-(2,3-dihydro-1,4-benzodioxin-6-yl)-3-iodo-4-oxo-4,5-dihydropyrazolo[1,5-a]pyrazine-2-carboxylate

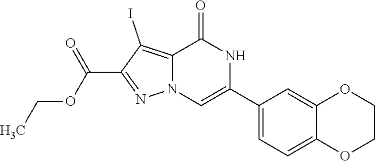 [SMILES: CCOC(=O)c1nn2cc(-c3ccc4c(c3)OCCO4)[nH]c(=O)c2c1I]

To a solution of diethyl 1-[2-(2,3-dihydro-1,4-benzodioxin-6-yl)-2-oxoethyl]-4-iodo-1H-pyrazole-3,5-dicarboxylate (Intermediate 346A, 7.00 g, 13.4 mmol) in acetic acid (120 ml) at room temperature was added ammonium acetate (20.6 g, 267 mmol) .The reaction was gradually warmed to 110° C. and the mixture was stirred at this temperature for 18 h. After cooling to RT, the solution was diluted with water (200 ml), the precipitate was collected by filtration, washed with water (50 ml) and dried to afford the title compound. Yield: 6.60 g (75% of theory, 71% purity).

LC/MS [Method 20]: Rₜ=0.94 min; MS (ESIpos): m/z=468 [M+H]⁺.

¹H-NMR (300 MHz, DMSO-d₆): δ [ppm]=11.61 (s, 1H), 8.11 (s, 1H), 7.29 (s, 1H), 7.25-7.22 (m, 1H), 6.96 (d, 1H), 4.35-4.29 (m, 6H), 1.34 (t, 3H).

### Intermediate 348A

### Ethyl 3-cyano-6-(2,3-dihydro-1,4-benzodioxin-6-yl)-4-oxo-4,5-dihydropyrazolo[1,5-a]pyrazine-2-carboxylate

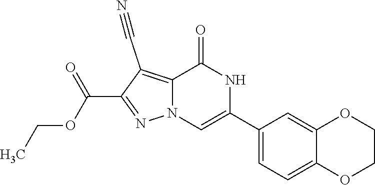 [SMILES: CCOC(=O)c1nn2cc(-c3ccc4c(c3)OCCO4)[nH]c(=O)c2c1C#N]

To a solution of ethyl ethyl 6-(2,3-dihydro-1,4-benzodioxin-6-yl)-3-iodo-4-oxo-4,5-dihydropyrazolo-[1,5-a]pyrazine-2-carboxylate (Intermediate 347A, 6.60 g, 9.96 mmol, 71% purity) in N,N-dimethylformamide (100 ml) was added copper(I) cyanide (1.78 g, 19.9 mmol). The reaction was gradually warmed to 150° C. and the mixture was stirred for 1 h. After cooling to RT, the insoluble material was filtered off. The filtrate was diluted with water (100 ml) and the precipitate was collected by filtration, washed with water (100 ml) and dried to afford the title compound. Yield: 6.20 g (99% of theory, 58% purity).

LC/MS [Method 10]: Rₜ=0.97 min; MS (ESIpos): m/z=367 [M+H]⁺.

### Intermediate 349A

### 3-Cyano-6-(2,3-dihydro-1,4-benzodioxin-6-yl)-4-oxo-4,5-dihydropyrazolo[1,5-a]pyrazine-2-carboxylic acid

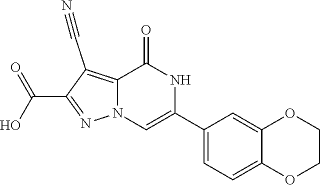 [SMILES: N#Cc1c(C(=O)O)nn2cc(-c3ccc4c(c3)OCCO4)[nH]c(=O)c12]

To a solution of ethyl 3-cyano-6-(2,3-dihydro-1,4-benzodioxin-6-yl)-4-oxo-4,5-dihydropyrazolo-[1,5-a]pyrazine-2-carboxylate (Intermediate 348A, 6.20 g, 9.82 mmol) in ethanol (50 ml) and water (40 ml) was added lithium hydroxide (2.35 g, 98.2 mmol) and the mixture was stirred at room temperature for 4 h. The solution was diluted with water (50 ml) and extracted with ethyl acetate (3×50 ml). The aqueous layer was adjusted to pH=3 with 3.0 M hydrochloric acid solution and the precipitate was collected by filtration and dried to afford the title compound. Yield: 1.00 g (29% of theory, 96% purity).

LC/MS [Method 39]: Rₜ=0.84 min; MS (ESIpos): m/z=339 [M+H]⁺.

¹H-NMR (400 MHz, DMSO-d₆): δ [ppm]=14.07 (br s, 1H), 12.16 (s, 1H), 8.26-8.18 (m, 1H), 7.39-7.11 (m, 2H), 6.95-6.91 (m, 1H), 4.31-4.25 (m, 4H).

### Intermediate 350A

### 3-Cyclopropyl-6-(3,4-dimethylphenyl)-7-iodo-4-oxo-4,5-dihydropyrazolo[1,5-a]pyrazine-2-carboxylic acid

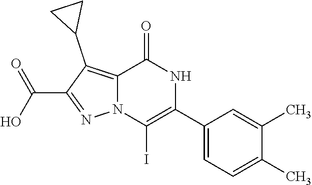 [SMILES: Cc1ccc(-c2[nH]c(=O)c3c(C4CC4)c(C(=O)O)nn3c2I)cc1C]

To a suspension of 3-cyclopropyl-6-(3,4-dimethylphenyl)-4-oxo-4,5-dihydropyrazolo[1,5-a]pyrazine-2-carboxylic acid (Intermediate 92A, 300 mg, 928 μmol) in DMF (18 ml) were added N-methylmorpholine (310 μl, 2.8 mmol) and N-iodosuccinimde (209 mg, 928 μmol). After stirring overnight at RT, the mixture was poured into water and 1.0 M hydrochloric acid was added until pH=3. The mixture was extracted with ethyl acetate and the combined organic layers were dried over magnesium sulfate and concentrated under reduced pressure. The residue was purified by preparative HPLC (Method P14) to afford the title compound. Yield: 88.3 mg (16% of theory, 75% purity).

LC/MS [Method 3]: Rₜ=1.67 min; MS (ESIpos): m/z=450 [M+H]⁺.

¹H-NMR (400 MHz, DMSO-d₆): δ [ppm]=13.17 (br s, 1H), 11.58 (s, 1H), 7.31-7.23 (m, 2H), 7.21 (d, 1H), 2.74-2.63 (m, 1H), 2.29 (s, 3H), 2.28 (s, 3H), 1.27-1.16 (m, 2H), 0.97-0.86 (m, 2H).

### Intermediate 351A

### 7-Cyano-3-cyclopropyl-6-(3,4-dimethylphenyl)-4-oxo-4,5-dihydropyrazolo[1,5-a]pyrazine-2-carboxylic acid

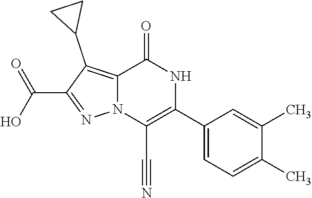 [SMILES: Cc1ccc(-c2[nH]c(=O)c3c(C4CC4)c(C(=O)O)nn3c2C#N)cc1C]

A solution of 3-cyclopropyl-6-(3,4-dimethylphenyl)-7-iodo-4-oxo-4,5-dihydropyrazolo[1,5-a]pyrazine-2-carboxylic acid (Intermediate 350A, 88.0 mg, 196 μmol) in N-methylpyrrolidone (1.7 ml) was treated with copper(I) cyanide and the mixture was heated to 150° C. in a microwave reactor for 1 h. After cooling to RT, the mixture was diluted with water and filtered. The filtrate was directly submitted to purification by preparative HPLC (Method P2) to afford the title compound. Yield: 10.0 mg (12% of theory, 75% purity).

LC/MS [Method 3]: Rt =1.59 min; MS (ESIneg): m/z=347 [M−H]⁻.

### Intermediate 352A

### (1S)-1-amino-1-(3,4-difluorophenyl)-2-methylpropan-2-ol

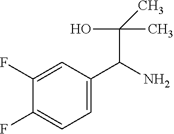

Methylmagnesium bromide (24 ml, 1.0 M solution in THF, 24 mmol) was added dropwise to a solution of methyl-amino(3,4-difluorophenyl)acetate (803 mg, 3.99 mmol) in THF (18 ml) at 0° C. and the mixture was allowed to warm to RT overnight. Then, 1.0 M hydrochloric acid was added carefully and the aqueous layer was washed with MTBE three times. The organic layer was discarded and the aqueous layer was basified by addition of 1.0 M aqueous sodium hydroxide solution. The precipitate was collected by filtration and washed with ethyl acetate. The aqueous layer was extracted with ethyl acetate and all ethyl acetate fractions were combined, dried over sodium sulfate and evaporated to dryness. The residue was purified by column chromatography (eluent: dichloromethane/7N ammonia in methanol 60:1 to 20:1) to afford the title compound (92.0 mg, 11% of theory).

LC/MS [Method 4]: Rₜ=1.10 min; MS (ESIpos): m/z=202 [M+H]⁺.

### Intermediate 353A

### 5-[(2,2-Difluorocyclopropyl)carbonyl]-2,2-dimethyl-1,3-dioxane-4,6-dione

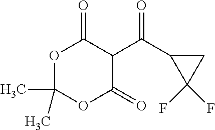 [SMILES: CC1(C)OC(=O)C(C(=O)C2CC2(F)F)C(=O)O1]

To a stirred solution of 2,2-difluorocyclopropanecarboxylic acid (14.0 g, 114.7 mmol) in DCM (150 mL) were added 4-dimethylaminopyridine (14.0 g, 114.7 mmol), 2,2-dimethyl-1,3-dioxane-4,6-dione (16.5 g, 114.7 mmol) and 1-(3-dimethylaminopropyl)-3-ethylcarbodiimide hydrochloride (21.9 g, 114.69 mmol) at 0° C. The reaction temperature was allowed to rise to RT. After stirring overnight, the reaction mixture was quenched with water (120 ml) and extracted with DCM (2×150 ml). The organic phase was washed with 1M sodium hydrogen sulfate (2×150 ml) and brine (2×150 ml) and dried over anhydrous sodium sulfate. The solid was filtered off. The filtrate was concentrated under reduced pressure. The residue was purified by flash chromatography on silica gel (eluent: DCM 100%) to give 20.0 g (63% of theory, 90% purity) of the title compound.

LC/MS [Method 42]: Rₜ=1.03 min; MS (ESIpos): m/z=247 [M−H]⁻.

¹H-NMR (300 MHz, DMSO-d₆): δ [ppm]=3.87-4.18 (m, 1H), 2.11-2.35 (m, 2H), 1.85-2.07 (m, 1H), 1.69 (s, 6H).

### Intermediate 354A

### Ethyl 3-(2,2-difluorocyclopropyl)-3-oxopropanoate

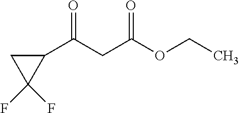

A solution of 5-[(2,2-difluorocyclopropyl)carbonyl]-2,2-dimethyl-1,3-dioxane-4,6-dione (Intermediate 353A, 20.0 g, 72.5 mmol, 90% purity) in ethanol (200 ml) was stirred for 3 h at 85° C. Then the reaction mixture was concentrated under reduced pressure. The residue was purified by flash chromatography on silica gel (eluent: DCM 100%) to give 17.4 g (99% of theory, 80% purity) of the title compound.

¹H-NMR (300 MHz, DMSO-d₆): δ [ppm]=4.04-4.17 (m, 2H), 3.58-3.88 (m, 2H), 3.18-3.27 (m, 1H), 1.97-2.10 (m, 2H), 1.19 (t, 3H).

### Intermediate 355A

### Ethyl (2Z)-3-(2,2-difluorocyclopropyl)-3-{[(trifluoromethyl)sulfonyl]oxy}acrylate

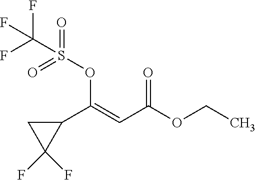 [SMILES: CCOC(=O)/C=C(\OS(=O)(=O)C(F)(F)F)C1CC1(F)F]

To a stirred solution of ethyl 3-(2,2-difluorocyclopropyl)-3-oxopropanoate (Intermediate 354A, 16.4 g, 76.8 mmol, 90% purity) in toluene (200 ml) was added a solution of lithium hydroxide (13.8 g, 576.1 mmol) in water (95 ml) at 0° C. After stirring for 10 minutes, trifluoromethanesulfonic anhydride (26 ml, 153.6 mmol) was added dropwise. After stirring for 2 h at RT, the reaction mixture was quenched with water (150 ml) and extracted with ethyl acetate (2×200 ml). The organic phase was washed with brine (2×200 ml) and dried over anhydrous sodium sulfate. The to solid was filtered off. The filtrate was concentrated under reduced pressure to give 22.0 g (72% of theory, 81% purity) of the title compound.

LC/MS [Method 42]: Rₜ=1.26 min; MS (ESIpos): m/z=325 [M+H]⁺.

¹H-NMR (400 MHz, DMSO-d₆): δ [ppm]=6.46 (d, 1H), 4.06-4.23 (m, 2H), 2.88-2.98 (td, 1H), 2.31-2.40 (m, 1H), 2.01-2.19 (m, 1H), 1.16-1.26 (m, 3H).

### Intermediate 356A

### Diethyl 4-(2,2-difluorocyclopropyl)-1H-pyrazole-3,5-dicarboxylate

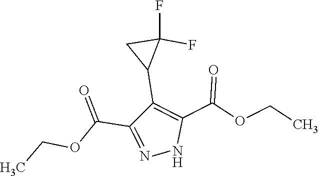 [SMILES: CCOC(=O)c1n[nH]c(C(=O)OCC)c1C1CC1(F)F]

To a stirred solution of ethyl (2Z)-3-(2,2-difluorocyclopropyl)-3-{[(trifluoromethyl)sulfonyl]oxy}-acrylate (Intermediate 355A, 22.0 g, 54.9 mmol, 81% purity) in N,N-dimethylformamide (200 ml) were added ethyl diazoacetate (9.4 g, 82.4 mmol) and 4-methylmorpholine (12.1 ml, 109.9 mmol) followed by tetrakis(triphenylphosphine)palladium(0) (3.2 g, 2.8 mmol) at RT. The resulting mixture was stirred for 3 h at RT under nitrogen and overnight at 60° C. The reaction mixture was quenched with water (150 ml) and extracted with ethyl acetate (2×200 ml). The organic phase was washed with brine and dried over anhydrous sodium sulfate. The solid was filtered off. The filtrate was concentrated under reduced pressure. The residue was purified by flash chromatography on silica gel (eluent: petroleum ether-ethyl acetate 100%, 85:15) to give 7.5 g of crude compound.

The crude product was triturated in petroleum ether-ethyl acetate (1:20) and stirred for 30 min. The solid was collected by filtration to give 5.75 g (36% of theory, 98% purity) of the title compound.

LC/MS [Method 43]: Rₜ=1.46 min; MS (ESIpos): m/z=289 [M+H]⁺.

¹H-NMR (400 MHz, DMSO-d₆): δ [ppm]=6.46 (d, 1H), 4.06-4.23 (m, 2H), 2.88-2.98 (td, 1H), 2.31-2.40 (m, 1H), 2.01-2.19 (m, 1H), 1.16-1.26 (m, 3H).

### Intermediate 357A

### Diethyl 1-[2-(4-chloro-3-methylphenyl)-2-oxoethyl]-4-(2,2-difluorocyclopropyl)-1H-pyrazole-3,5-dicarboxylate

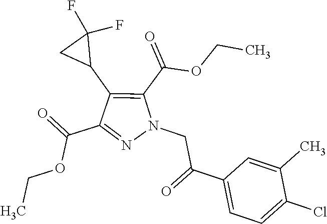 [SMILES: CCOC(=O)c1nn(CC(=O)c2ccc(Cl)c(C)c2)c(C(=O)OCC)c1C1CC1(F)F]

To a solution of diethyl 4-(2,2-difluorocyclopropyl)-1H-pyrazole-3,5-dicarboxylate (Intermediate 356A, 2.0 g, 6.8 mmol, 98% purity) and 2-bromo-1-(4-chloro-3-methylphenyl)ethanone (2.18 g, 7.48 mmol, 85% purity) in acetone (20 ml) was added potassium carbonate (2.8 g, 20.4 mmol). The resulting mixture was stirred overnight at RT. After filtering off the solids, the filter cake was washed with ethyl acetate and the filtrate was concentrated under reduced pressure. The residue was purified by flash chromatography on silica gel (eluent: petroleum ether-ethyl acetate 100%, 5: 1) to give 2.4 g (75% of theory, 97% purity) of the title compound.

LC/MS [Method 40]: Rₜ=1.34 min; MS (ESIpos): m/z=455 [M+H]⁺.

¹H-NMR (300 MHz, DMSO-d₆): δ [ppm]=8.05 (d, 1H), 7.89 (dd, 1H), 7.66 (d, 1H), 6.20 (s, 2H), 4.14-4.37 (m, 4H), 2.84-2.95 (m, 1H), 2.44 (s, 3H), 2.03-2.16 (m, 1H), 1.54-1.64 (m, 1H), 1.32 (t, 3H), 1.16-1.22 (m, 3H).

### Intermediate 358A

### Ethyl 6-(4-chloro-3-methylphenyl)-3-(2,2-difluorocyclopropyl)-4-oxo-4,5-dihydropyrazolo[1,5-a]pyrazine-2-carboxylate

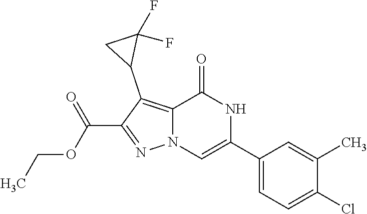 [SMILES: CCOC(=O)c1nn2cc(-c3ccc(Cl)c(C)c3)[nH]c(=O)c2c1C1CC1(F)F]

To a solution of diethyl 1-[2-(4-chloro-3-methylphenyl)-2-oxoethyl]-4-(2,2-difluorocyclopropyl)-1H-pyrazole-3,5-dicarboxylate (Intermediate 357A, 1.85 g, 3.95 mmol, 97% purity) in acetic acid (50 ml) was added ammonium acetate (9.1 g, 118.3 mmol). The resulting mixture was heated overnight at 110° C. After cooled to RT, the reaction mixture was poured into ice-water. The solid was collected by filtration, washed with water and dried in vacuum to give 1.3 g (75% of theory, 92% purity) of the title compound.

¹H-NMR (400 MHz, DMSO-d₆): δ [ppm]=11.68 (s, 1H), 8.17 (s, 1H), 7.79 (d, 1H), 7.60 (dd, 1H), 7.54 (d, 1H), 4.36 (q, 2H), 3.02-3.10 (m, 1H), 2.39 (s, 3H), 2.03-2.16 (m, 2H), 1.35 (t, 3H).

¹⁹F-NMR (376 MHz, DMSO-d₆): δ [ppm]=−128.09 (d, 1F), -137.29 (d, 1F).

### Intermediate 359A

### 6-(4-Chloro-3-methylphenyl)-3-(2,2-difluorocyclopropyl)-4-oxo-4,5-dihydropyrazolo[1,5-a]-pyrazine-2-carboxylic acid

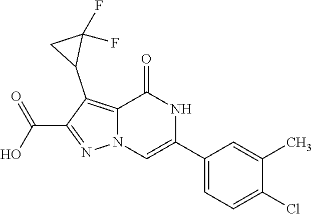 [SMILES: Cc1cc(-c2cn3nc(C(=O)O)c(C4CC4(F)F)c3c(=O)[nH]2)ccc1Cl]

To a mixture of ethyl 6-(4-chloro-3-methylphenyl)-3-(2,2-difluorocyclopropyl)-4-oxo-4,5-dihydro-pyrazolo[1,5-a]pyrazine-2-carboxylate (Intermediate 358A, 1.3 g, 2.9 mmol, 92% purity) in ethanol (15 ml) was added a solution of sodium hydroxide (1.2 g, 29.8 mmol) in water (4 ml). After stirring at RT for 3 h, the reaction mixture was concentrated under reduced pressure to remove the ethanol. Then the rest of mixture was diluted with water (10 ml) and extracted with MTBE (2×10 mL). The aqueous was acidified with 2M hydrochloric acid to pH=1. The product was collected by filtration, washed with water and dried in air to give 1.08 g (91% of theory, 95% purity) of the title compound.

LC/MS [Method 50]: Rₜ=1.49 min; MS (ESIpos): m/z=402 [M+Na]⁺.

¹H-NMR (400 MHz, DMSO-d₆): δ [ppm]=11.59 (s, 1H), 8.11 (s, 1H), 7.81 (d, 1H), 7.60 (dd, 1H), 7.52 (d, 1H), 3.13-3.29 (m, 1H), 2.41 (s, 3H), 2.23-2.39 (m, 1H), 2.02-2.09 (m, 1H).

¹⁹F-NMR (376 MHz, DMSO-d₆): δ [ppm]=−127.70 (d, 1F), -137.90 (d, 1F).

### Intermediate 360A

### 5-[(3,3-Difluorocyclobutyl)carbonyl]-2,2-dimethyl-1,3-dioxane-4,6-dione

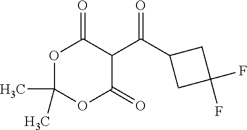 [SMILES: CC1(C)OC(=O)C(C(=O)C2CC(F)(F)C2)C(=O)O1]

To a stirred solution of 3,3-difluorocyclobutanecarboxylic acid (14.7 g, 108.0 mmol) in DCM (150 ml) were added 4-dimethylaminopyridine (13.2 g, 108.0 mmol), 2,2-dimethyl-1,3-dioxane-4,6-dione (15.5 g, 108.0 mmol) and 1-(3-dimethylaminopropyl)-3-ethylcarbodiimide hydrochloride (20.7 g, 108.0 mmol) at 0° C. The reaction temperature was allowed to rise to RT. After stirring overnight, the reaction mixture was quenched with water (100 ml) and extracted with DCM (2×100 ml). The organic phase was washed with 1M sodium hydrogen sulfate (2×100 ml) and brine (2×100 ml) and dried over anhydrous sodium sulfate. The solid was filtered off. The filtrate was concentrated under reduced pressure. The residue was purified by flash chromatography on silica gel (eluent: DCM 100%) to give 13.0 g (41% of theory, 90% purity) of the title compound.

LC/MS [Method 42]: Rₜ=1.15 min; MS (ESIpos): m/z=261 [M−H]⁻.

### Intermediate 361A

### Ethyl 3-(3,3-difluorocyclobutyl)-3-oxopropanoate

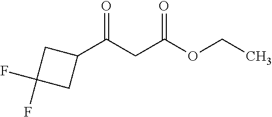

A solution of 5-[(3,3-difluorocyclobutyhcarbonyI]-2,2-dimethyl-1,3-dioxane-4,6-dione (Intermediate 360A, 13.0 g, 44.6 mmol, 90% purity) in ethanol (120 ml) was stirred for 3 h at 85° C. Then the reaction mixture was concentrated under reduced pressure. The residue was purified by flash chromatography on silica gel (eluent: DCM 100%) to give 8.0 g (83% of theory, 96% purity) of the title compound.

LC/MS [Method 43]: Rₜ=1.38 min; MS (ESIpos): m/z=207 [M+H]⁺.

¹H-NMR (300 MHz, DMSO-d₆): δ [ppm]=4.11 (q, 2H), 3.68 (s, 2H), 3.28-3.37 (m, 1H), 2.68-2.84 (m, 4H), 1.18-1.25 (m, 3H).

### Intermediate 362A

### Ethyl (2Z)-3-(3,3-difluorocyclobutyl)-3-{[(trifluoromethyl)sulfonyl]oxy}acrylate

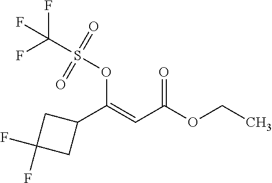 [SMILES: CCOC(=O)/C=C(\OS(=O)(=O)C(F)(F)F)C1CC(F)(F)C1]

To a stirred solution of ethyl 3-(3,3-difluorocyclobutyl)-3-oxopropanoate (Intermediate 361A, 8.00 g, 37.25 mmol, 96% purity) in toluene (100 ml) was added a solution of lithium hydroxide (11.7 g, 279.3 mmol) in water (48 ml) at 0° C. After stirring for 10 min, trifluoromethanesulfonic anhydride (12.6 ml, 74.5 mmol) was added dropwise. After stirring for 2 h at RT, the reaction mixture was quenched with water (80 ml) and extracted with ethyl acetate (2×100 ml). The organic phase was washed with brine (2×100 ml) and dried over anhydrous sodium sulfate. The solid was filtered off. The filtrate was concentrated under reduced pressure to give 11.0 g (83% of theory, 95% purity) of the title compound.

LC/MS [Method 42]: Rₜ=1.28 min; MS (ESIpos): m/z=339 [M+H]⁺.

### Intermediate 363A

### Diethyl 4-(3,3-difluorocyclobutyl)-1H-pyrazole-3,5-dicarboxylate

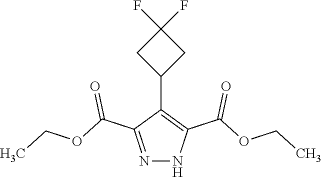 [SMILES: CCOC(=O)c1n[nH]c(C(=O)OCC)c1C1CC(F)(F)C1]

To a stirred solution of ethyl (2Z)-3-(3,3-difluorocyclobutyl)-3-{[(trifluoromethyl)sulfonyl]oxy}-acrylate (Intermediate 362A, 11.0 g, 29.3 mmol, 90% purity) in N,N-dimethylformamide (100 ml) is were added ethyl diazoacetate (5.0 g, 43.9 mmol) and 4-methylmorpholine (6.4 ml, 58.5 mmol) followed by tetrakis(triphenylphosphine)palladium(0) (1.7 g, 1.5 mmol) at RT. The resulting mixture was stirred for 3 h at RT under nitrogen and overnight at 60° C. The reaction mixture was quenched with water (100 ml) and extracted with ethyl acetate (2×100 ml). The organic phase was washed with brine and dried over anhydrous sodium sulfate. The solid was filtered off. The filtrate was concentrated under reduced pressure. The residue was purified by flash chromatography on silica gel (eluent: petroleum ether-ethyl acetate 100%, 77:23) to give 5.1 g (52% of theory, 91% purity) of the title compound.

LC/MS [Method 51]: Rₜ=1.36 min; MS (ESIpos): m/z=303 [M+H]⁺.

¹H-NMR (300 MHz, DMSO-d₆): δ [ppm]=14.44 (s, 1H), 4.13-4.35 (m, 4H), 4.10-4.20 (m, 1H), 3.01-3.19 (m, 2H), 2.71-2.83 (m, 2H), 1.09-1.33 (m, 6H).

¹⁹F-NMR (282 MHz, DMSO-d₆): δ [ppm]=−80.45 (d, 1F), −100.59 (d, 1F).

### Intermediate 364A

### Diethyl 1-[2-(3,4-dichlorophenyl)-2-oxoethyl]-4-(3,3-difluorocyclobutyl)-1H-pyrazole-3,5-dicarboxylate

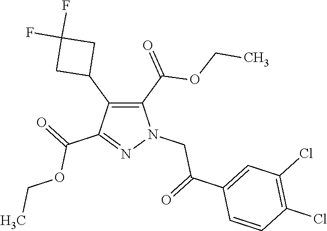 [SMILES: CCOC(=O)c1nn(CC(=O)c2ccc(Cl)c(Cl)c2)c(C(=O)OCC)c1C1CC(F)(F)C1]

To a solution of diethyl 4-(3,3-difluorocyclobutyl)-1H-pyrazole-3,5-dicarboxylate (Intermediate 363A, 600.0 mg, 1.9 mmol) and 2-bromo-1-(3,4-dichlorophenyl)ethanone (651.2 mg, 2.4 mmol, 98% purity) in acetone (17.4 ml) was added potassium carbonate (685.8 mg, 4.9 mmol). The resulting mixture was stirred for 1 h at RT. After filtering off the solids, the filter cake was washed with acetone and the filtrate was concentrated under reduced pressure. The residue was purified by flash chromatography on silica gel (eluent: cyclohexane-ethyl acetate 7:3) to give 886 mg (91% of theory, 100% purity) of the title compound.

LC/MS [Method 3]: Rₜ=2.45 min; MS (ESIpos): m/z=489 [M+H]⁺.

¹H-NMR (400 MHz, DMSO-d₆): δ [ppm]=8.29 (d, 1H), 8.00 (dd, 1H), 7.90 (d, 1H), 6.23 (s, 2H), 4.34 (q, 2H), 4.21 (q, 2H), 4.18-4.09 (m, 1H), 3.16-3.02 (m, 2H), 2.87-2.77 (m, 2H), 1.31 (t, 3H), 1.10 (t, 3H).

### Intermediate 365A

### Ethyl 6-(3,4-dichlorophenyl)-3-(3,3-difluorocyclobutyl)-4-oxo-4,5-dihydropyrazolo[1,5-a]pyrazine-2-carboxylate

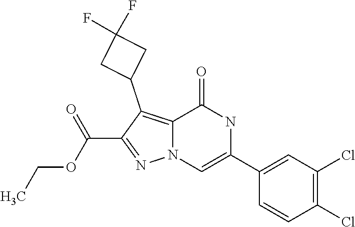 [SMILES: CCOC(=O)c1nn2cc(-c3ccc(Cl)c(Cl)c3)[nH]c(=O)c2c1C1CC(F)(F)C1]

To a solution of diethyl 1-[2-(3,4-dichlorophenyl)-2-oxoethyl]-4-(3,3-difluorocyclobutyl)-1H-pyrazole-3,5-dicarboxylate (Intermediate 364A, 886 mg, 1.8 mmol) in acetic acid (15 ml) was added ammonium acetate (1.4 g, 18.1 mmol). The resulting mixture was heated overnight at 110° C. After cooled to RT, the reaction mixture was quenched with water. The solid was collected by filtration, washed with water and dried in vacuum to give 748.0 mg (92% of theory, 98% purity) of the title compound.

LC/MS [Method 3]: Rₜ=2.28 min; MS (ESIpos): m/z=442 [M+H]⁺.

¹H-NMR (600 MHz, DMSO-d₆): δ [ppm]=11.81 (s, 1H), 8.31 (s, 1H), 8.07 (d, 1H), 7.77 8q, 2H), 4.42-4.35 (m, 3H), 3.35-3.25 (m, 1H), 2.85-2.79 (m, 2H), 1.91 (s, 1H), 1.35-1.31 (m, 3H).

### Intermediate 366A

### 6-(3,4-dichlorophenyl)-3-(3,3-difluorocyclobutyl)-4-oxo-4,5-dihydropyrazolo[1 ,5-a]pyrazine-2-carboxylic acid

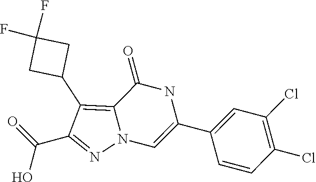 [SMILES: O=C(O)c1nn2cc(-c3ccc(Cl)c(Cl)c3)[nH]c(=O)c2c1C1CC(F)(F)C1]

To a mixture of ethyl 6-(3,4-dichlorophenyl)-3-(3,3-difluorocyclobutyl)-4-oxo-4,5-dihydropyrazolo-[1,5-a]pyrazine-2-carboxylate (Intermediate 365A, 745.0 mg, 1.6 mmol, 98% purity) in a mixture THF/methanol (18 ml, 5/1) was added a solution 1M of lithium hydroxide (8.2 ml, 8.2 mmol). After stirring at RT for 3.5 h, the reaction mixture was concentrated under reduced pressure. Then the rest of mixture was diluted with water and acidified with 2M hydrochloric acid. The product was collected by filtration, washed with water and dried in air to give 671.0 mg (98% of theory, 100% purity) of the title compound.

LC/MS [Method 3]: Rₜ=1.83 min; MS (ESIpos): m/z=412 [M−H]⁻.

¹H-NMR (600 MHz, DMSO-d₆): δ [ppm]=11.77 (s, 1H), 8.24 (s, 1H), 8.07 (s, 1H), 7.77 (q, 2H), 4.42 (t, 1H), 3.29 (m, 2H), 2.81 (m, 2H).

### Intermediate 367A

### Diethyl 1-[2-(4-chloro-3-fluorophenyl)-2-oxoethyl]-4-(3,3-difluorocyclobutyl)-1H-pyrazole-3,5-dicarboxylate

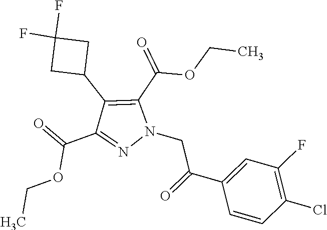 [SMILES: CCOC(=O)c1nn(CC(=O)c2ccc(Cl)c(F)c2)c(C(=O)OCC)c1C1CC(F)(F)C1]

To a solution of diethyl 4-(3,3-difluorocyclobutyl)-1H-pyrazole-3,5-dicarboxylate (Intermediate 363A, 300.0 mg, 0.9 mmol) and 2-bromo-1-(4-chloro-3-fluorophenyl)ethanone (Intermediate 255A, 406.9 mg, 1.2 mmol, 74% purity) in acetone (8.7 ml) was added potassium carbonate (342.9 mg, 2.5 mmol). The resulting mixture was stirred for 1 h at RT. After filtering off the solids, the filter cake was washed with acetone and the filtrate was concentrated under reduced pressure to give 348 mg (60% of theory, 81% purity) of the title compound.

LC/MS [Method 3]: Rₜ=2.35 min; MS (ESIpos): m/z=473 [M+H]⁺.

### Intermediate 368A

### Ethyl 6-(4-chloro-3-fluorophenyl)-3-(3,3-difluorocyclobutyl)-4-oxo-4,5-dihydropyrazolo[1,5-a]pyrazine-2-carboxylate

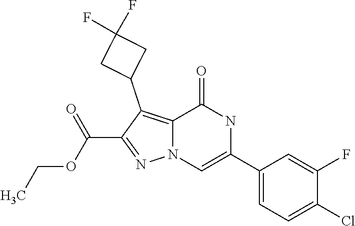 [SMILES: CCOC(=O)c1nn2cc(-c3ccc(Cl)c(F)c3)[nH]c(=O)c2c1C1CC(F)(F)C1]

To a solution of diethyl 1-[2-(4-chloro-3-fluorophenyl)-2-oxoethyl]-4-(3,3-difluorocyclobutyl)-1H-pyrazole-3,5-dicarboxylate (Intermediate 367A, 348.0 mg, 0.7 mmol) in acetic acid (6.2 ml) was to added ammonium acetate (567.3 mg, 7.4 mmol). The resulting mixture was heated overnight at 110° C. After cooled to RT, the reaction mixture was quenched with water. The solid was collected by filtration, washed with water and dried in vacuum to give 227.0 mg (71% of theory, 98% purity) of the title compound.

LC/MS [Method 11]: Rₜ=1.12 min; MS (ESIpos): m/z=426 [M+H]⁺.

### Intermediate 369A

### 6-(4-Chloro-3-fluorophenyl)-3-(3,3-difluorocyclobutyl)-4-oxo-4,5-dihydropyrazolo[1,5-a]pyrazine-2-carboxylic acid

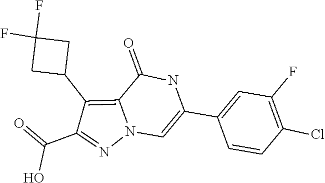 [SMILES: O=C(O)c1nn2cc(-c3ccc(Cl)c(F)c3)[nH]c(=O)c2c1C1CC(F)(F)C1]

To a mixture of ethyl 6-(4-chloro-3-fluorophenyl)-3-(3,3-difluorocyclobutyl)-4-oxo-4,5-dihydropyrazolo[1,5-a]pyrazine-2-carboxylate (Intermediate 368A, 227.0 mg, 0.5 mmol, 98% purity) in a mixture THF/methanol (4 ml, 5/1) was added a solution 1M of lithium hydroxide (2.7 ml, 2.7 mmol). After stirring overnight at RT, the reaction mixture was concentrated under reduced pressure. Then the rest of mixture was diluted with water and acidified with 1N hydrochloric acid. The product was collected by filtration, washed with water and dried in air to give 156.6 mg (72% of theory, 98% purity) of the title compound.

LC/MS [Method 3]: Rₜ=1.73 min; MS (ESIpos): m/z=396 [M−H]⁻.

¹H-NMR (400 MHz, DMSO-d₆): δ [ppm]=13.39 (bs, 1H), 11.76 (s, 1H), 8.25 (d, 1H), 7.90-7.86 (dd, 1H), 7.74 (m, 1H), 7.66 (m, 1H), 4.41 (m, 1H), 3.28 (m, 2H), 2.85-2.76 (m, 2H).

### Intermediate 370A

### Diethyl 1-[2-(4-chloro-3-methylphenyl)-2-oxoethyl]-4-(3,3-difluorocyclobutyl)-1H-pyrazole-3,5-dicarboxylate

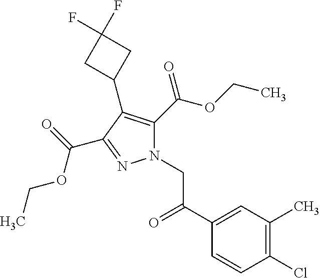 [SMILES: CCOC(=O)c1nn(CC(=O)c2ccc(Cl)c(C)c2)c(C(=O)OCC)c1C1CC(F)(F)C1]

To a solution of diethyl 4-(3,3-difluorocyclobutyl)-1H-pyrazole-3,5-dicarboxylate (Intermediate 363A, 600.0 mg, 1.9 mmol) and 2-bromo-1-(4-chloro-3-methylphenyl)ethanone (1.6 g, 6.1 mmol, 95% purity) in acetone (17 ml) was added potassium carbonate (685.8 mg, 4.9 mmol). The resulting mixture was stirred at RT for 1 h. After filtering off the solids, the filter cake was washed with acetone and the filtrate was concentrated under reduced pressure to give 1.0 g (97% of theory, 90% purity) of the title compound.

LC/MS [Method 3]: Rₜ=2.43 min; MS (ESIpos): m/z=469 [M+H]⁺.

### Intermediate 371A

### Ethyl 6-(4-chloro-3-methylphenyl)-3-(3,3-difluorocyclobutyl)-4-oxo-4,5-dihydropyrazolo[1,5-a]-pyrazine-2-carboxylate

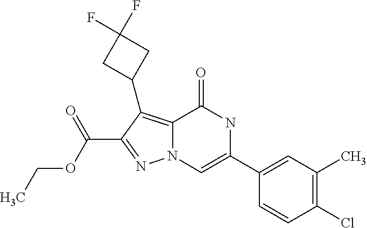 [SMILES: CCOC(=O)c1nn2cc(-c3ccc(Cl)c(C)c3)[nH]c(=O)c2c1C1CC(F)(F)C1]

To a solution of diethyl 1-[2-(4-chloro-3-methylphenyl)-2-oxoethyl]-4-(3,3-difluorocyclobutyl)-1H-pyrazole-3,5-dicarboxylate (Intermediate 370A, 1.0 g, 1.9 mmol) in acetic acid (8.1 ml) was added ammonium acetate (1.5 g, 19.2 mmol). The resulting mixture was heated for two days at 110° C. After cooled to RT, the reaction mixture was quenched with water. The solid was collected by filtration, washed with water and dried in vacuum to give 432.0 mg (52% of theory, 98% purity) of the title compound.

LC/MS [Method 3]: Rₜ=2.28 min; MS (ESIpos): m/z=420 [M−H]⁻.

### Intermediate 372A

### 6-(4-Chloro-3-methylphenyl)-3-(3,3-difluorocyclobutyl)-4-oxo-4,5-dihydropyrazolo[1,5-a]pyrazine-2-carboxylic acid

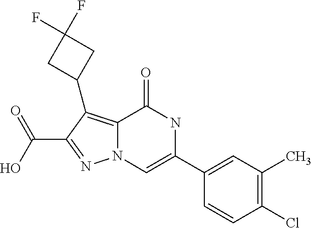 [SMILES: Cc1cc(-c2cn3nc(C(=O)O)c(C4CC(F)(F)C4)c3c(=O)[nH]2)ccc1Cl]

To a mixture of ethyl 6-(4-chloro-3-methylphenyl)-3-(3,3-difluorocyclobutyl)-4-oxo-4,5-dihydropyrazolo[1,5-a]pyrazine-2-carboxylate (Intermediate 371A, 432.0 mg, 1.0 mmol, 98% purity) in a mixture THF/methanol (7.7 ml, 5/1) was added a solution 1M of lithium hydroxide (5.1 ml, 5.1 mmol). After stirring overnight at RT, the reaction mixture was concentrated under reduced pressure. Then the rest of mixture was diluted with water and acidified with 1N hydrochloric acid. The product was collected by filtration, washed with water and dried in high vacuum to give 340.0 mg (80% of theory, 95% purity) of the title compound.

LC/MS [Method 3]: Rt =1.79 min; MS (ESIpos): m/z=392 [M−H]⁻.

¹H-NMR (600 MHz, DMSO-d₆): δ [ppm]=13.38 (bs, 1H), 11.66 (s, 1H), 8.10 (bs, 1H), 7.77 (bs, 1H), 7.59 (bd, 1H), 7.51 (bd, 1H), 4.43 (m, 1H), 3.30 (m, 2H), 2.81 (m, 2H), 2.38 (s, 3H).

### Intermediate 373A

### Diethyl 1-[2-(3-chloro-4-methylphenyl)-2-oxoethyl]-4-(3,3-difluorocyclobutyl)-1H-pyrazole-3,5-dicarboxylate

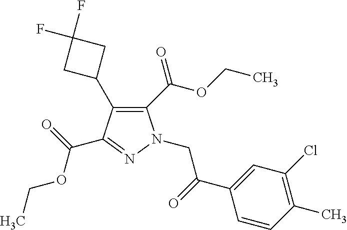 [SMILES: CCOC(=O)c1nn(CC(=O)c2ccc(C)c(Cl)c2)c(C(=O)OCC)c1C1CC(F)(F)C1]

To a solution of diethyl 4-(3,3-difluorocyclobutyl)-1H-pyrazole-3,5-dicarboxylate (Intermediate 363A, 1.8 g, 5.6 mmol, 91% purity) and 2-bromo-1-(3-chloro-4-methylphenyl)ethanone (1.6 g, 6.1 mmol, 95% purity) in acetone (40 ml) was added potassium carbonate (2.3 g, 16.7 mmol). The resulting mixture was stirred at RT for 2 h. After filtering off the solids, the filter cake was washed with acetone and the filtrate was suspended in a mixture ethyl acetate-n-Hexane (60 ml, 1:20) and stirred for 30 minutes. The solid was collected by filtration to give 1.8 g (61% of theory, 90% purity) of the title compound.

LC/MS [Method 43]: Rₜ=2.08 min; MS (ESIpos): m/z=469 [M+H]⁺.

¹H-NMR (300 MHz, DMSO-d₆): δ [ppm]=8.03 (d, 1H), 7.89 (dd, 1H), 7.57 (d, 1H), 6.17 (s, 2H), 4.31 (q, 2H), 4.05-4.21 (m, 3H), 2.97-3.12 (m, 2H), 2.74-2.87 (m, 2H), 2.42 (s, 3H), 1.29 (t, 3H), 1.07 (t, 3H).

### Intermediate 374A

### 6-(3-Chloro-4-methylphenyl)-3-(3,3-difluorocyclobutyl)-4-oxo-4,5-dihydropyrazolo[1,5-a]pyrazine-2-carboxylate

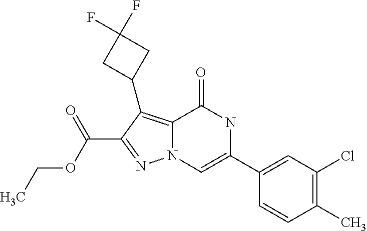 [SMILES: CCOC(=O)c1nn2cc(-c3ccc(C)c(Cl)c3)[nH]c(=O)c2c1C1CC(F)(F)C1]

To a solution of diethyl 1-[2-(3-chloro-4-methylphenyl)-2-oxoethyl]-4-(3,3-difluorocyclobutyl)-1H-pyrazole-3,5-dicarboxylate (Intermediate 373A, 1.9 g, 3.6 mmol, 90% purity) in acetic acid (60 ml) was added ammonium acetate (8.2 g, 106.5 mmol). The resulting mixture was heated overnight at 110° C. After cooled to RT, the reaction mixture was quenched with water. The solid was collected by filtration, washed with water and dried in vacuum to give 1.6 g (96% of theory, 90% purity) of the title compound.

LC/MS [Method 43]: Rₜ=1.97 min; MS (ESIpos): m/z=422 [M+H]⁺.

¹H-NMR (300 MHz, DMSO-d₆): δ [ppm]=11.72 (s, 1H), 8.19 (s, 1H), 7.82 (d, 1H), 7.61 (d, 1H), 7.44 (d, 1H), 4.32-4.37 (m, 3H), 2.77-2.81 (m, 2H), 2.35 (s, 3H), 1.88 (s, 2H), 1.31 (t, 3H).

### Intermediate 375A

### 6-(3-Chloro-4-methylphenyl)-3-(3,3-difluorocyclobutyl)-4-oxo-4,5-dihydropyrazolo[1,5-a]pyrazine-2-carboxylic acid

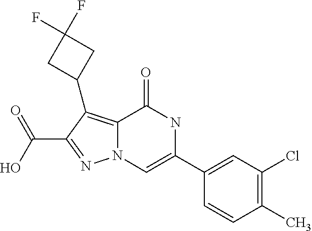 [SMILES: Cc1ccc(-c2cn3nc(C(=O)O)c(C4CC(F)(F)C4)c3c(=O)[nH]2)cc1Cl]

To a mixture of 6-(3-chloro-4-methylphenyl)-3-(3,3-difluorocyclobutyl)-4-oxo-4,5-dihydropyrazolo-[1,5-a]pyrazine-2-carboxylate (Intermediate 374A, 1.6 g, 3.4 mmol, 90% purity) in ethanol (60 ml) was added a solution of sodium hydroxide (1.4 g, 34.1 mmol) in water (20 ml). After stirring at RT for 2 h, the reaction mixture was concentrated under reduced pressure. Then the rest of mixture was diluted with water and acidified with 4N hydrochloric acid. The product was collected by filtration, washed with water and dried in high vacuum to give 1.37 g (99% of theory, 98% purity) of the title compound.

LC/MS [Method 43]: Rₜ=1.67 min; MS (ESIpos): m/z=416 [M+Na]⁺.

¹H-NMR (300 MHz, DMSO-d₆): δ [ppm]=11.71 (s, 1H), 8.14 (s, 1H), 7.84 (d, 1H), 7.62 (dd, 1H), 7.45(d, 1H), 4.33-4.48 (m, 1H), 3.20-3.37 (m, 2H), 2.77-2.85 (m, 2H), 2.37 (s, 3H).

¹⁹F-NMR (282 MHz, DMSO-d₆): δ [ppm]=−101.16 (d, 1F), -8039 (d, 1F).

### Intermediate 376A

### Diethyl 1-[2-(3-chloro-4-methylphenyl)-2-oxoethyl]-4-(2,2-difluorocyclopropyl)-1H-pyrazole-3,5-dicarboxylate

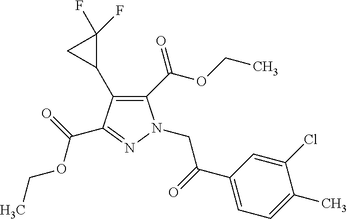 [SMILES: CCOC(=O)c1nn(CC(=O)c2ccc(C)c(Cl)c2)c(C(=O)OCC)c1C1CC1(F)F]

To a solution of diethyl 4-(2,2-difluorocyclopropyl)-1H-pyrazole-3,5-dicarboxylate (Intermediate 356A, 2.0 g, 5.9 mmol, 85% purity) and 2-bromo-1-(3-chloro-4-methylphenyl)ethanone (1.57 g, 5.9 mmol, 93% purity) in acetone (20 ml) was added potassium carbonate (1.6 g, 11.8 mmol). The resulting mixture was stirred overnight at RT. After filtering off the solids, the filter cake was washed with ethyl acetate and the filtrate was concentrated under reduced pressure. The residue was purified by flash chromatography on silica gel (eluent: petroleum ether-ethyl acetate 100%, 88:12) to give 2.1 g (73% of theory, 93% purity) of the title compound. LC/MS [Method 12]: Rₜ=1.27 min; MS (ESIpos): m/z=455 [M+H]⁺.

¹H-NMR (300 MHz, DMSO-d₆): δ [ppm]=8.04 (d, 1H), 7.90 (dd, 1H), 7.58 (d, 1H), 6.19 (s, 2H), 4.15-4.34 (m, 4H), 2.82-2.93 (m, 1H), 2.43 (s, 3H), 2.04-2.13 (m, 1H), 1.51-1.61 (m, 1H), 1.30 (t, 3H), 1.15-1.20 (m, 3H).

### Intermediate 377A

### Ethyl 6-(3-chloro-4-methylphenyl)-3-(2,2-difluorocyclopropyl)-4-oxo-4,5-dihydropyrazolo[1,5-a]-pyrazine-2-carboxylate

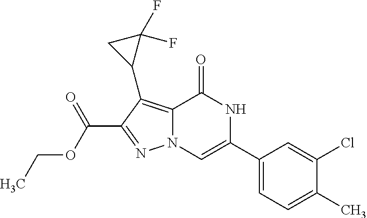 [SMILES: CCOC(=O)c1nn2cc(-c3ccc(C)c(Cl)c3)[nH]c(=O)c2c1C1CC1(F)F]

To a solution of diethyl 1-[2-(3-chloro-4-methylphenyl)-2-oxoethyl]-4-(2,2-difluorocyclopropyl)-1H-pyrazole-3,5-dicarboxylate (Intermediate 376A, 2.1 g, 4.3 mmol, 93% purity) in acetic acid (20 ml) was added ammonium acetate (13.2 g, 171.7 mmol). The resulting mixture was heated overnight at 110° C. After cooled to RT, the reaction mixture was quenched with water. The solid was collected by filtration, washed with water and dried in vacuum to give 1.6 g (83% of theory, 91% purity) of the title compound.

LC/MS [Method 44]: Rₜ=1.13 min; MS (ESIpos): m/z=408 [M+H]⁺.

¹H-NMR (300 MHz, DMSO-d₆): δ [ppm]=11.69 (s, 1H), 8.19 (s, 1H), 7.83 (d, 1H), 7.62 (dd, 1H), 7.45 (d, 1H), 4.34 (q, 2H), 2.98-3.09 (m, 1H), 2.36 (s, 3H), 2.00-2.15 (m, 2H), 1.32 (t, 3H).

### Intermediate 378A

### 6-(3-Chloro-4-methylphenyl)-3-(2,2-difluorocyclopropyl)-4-oxo-4,5-dihydropyrazolo[1,5-a]pyrazine-2-carboxylic acid

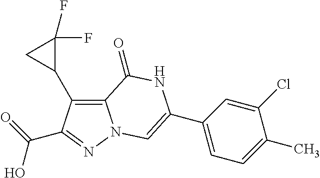 [SMILES: Cc1ccc(-c2cn3nc(C(=O)O)c(C4CC4(F)F)c3c(=O)[nH]2)cc1Cl]

To a mixture of ethyl 6-(3-chloro-4-methylphenyl)-3-(2,2-difluorocyclopropyl)-4-oxo-4,5-dihydropyrazolo[1,5-a]pyrazine-2-carboxylate (Intermediate 377A, 1.6 g, 3.6 mmol, 91% purity) in ethanol (30 ml) was added a solution of sodium hydroxide (1.4 g, 35.7 mmol) in water (10 ml). After stirring overnight at 40° C., the reaction mixture was concentrated under reduced pressure to remove ethanol. Then the rest of mixture was diluted with water (10 ml) and extracted with MTBE (2×30 mL). The aqueous was acidified with 2M hydrochloric acid to pH=1. The product was collected by filtration, washed with water and dried in air to give 1.2 g (91% of theory, 99% purity) of the title compound.

LC/MS [Method 52]: Rₜ=1.34 min; MS (ESIpos): m/z=380 [M+H]⁺.

¹H-NMR (400 MHz, DMSO-d₆): δ [ppm]=11.64 (s, 1H), 8.17 (s, 1H), 7.84 (s, 1H), 7.64 (d, 1H), 7.46 (d, 1H), 3.08-3.19 (m, 1H), 2.38 (s, 3H), 2.20 (s, 1H), 2.02-2.11 (m, 1H).

¹⁹F-NMR (376 MHz, DMSO-d₆): δ [ppm]=−132.72 (d, 1F), −127.80 (d, 1F).

### Intermediate 379A

### Diethyl 1-[2-(4-chloro-3-fluorophenyl)-2-oxoethyl]-4-(2,2-difluorocyclopropyl)-1H-pyrazole-3,5-dicarboxylate

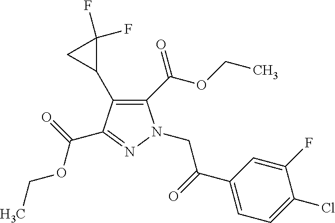 [SMILES: CCOC(=O)c1nn(CC(=O)c2ccc(Cl)c(F)c2)c(C(=O)OCC)c1C1CC1(F)F]

To a solution of diethyl 4-(2,2-difluorocyclopropyl)-1H-pyrazole-3,5-dicarboxylate (Intermediate 356A, 250.0 mg, 0.86 mmol) and 2-bromo-1-(4-chloro-3-fluorophenyl)ethanone (Intermediate 255A, 261.7 mg, 1.04 mmol) in acetone (7.6 ml) was added potassium carbonate (299.6 mg, 2.2 mmol). The resulting mixture was stirred at RT for 2h. After filtering off the solids, the filter cake was washed with acetone and the filtrate was concentrated under reduced pressure. The residue was purified by flash chromatography on silica gel (eluent: cyclohexane-ethyl acetate 100%, 7:3) to give 383.0 mg (96% of theory, 100% purity) of the title compound.

LC/MS [Method 11]: Rt =1.16 min; MS (ESIpos): m/z=459 [M+H]⁺.

### Intermediate 380A

### Ethyl 6-(4-chloro-3-fluorophenyl)-3-(2,2-difluorocyclopropyl)-4-oxo-4,5-dihydropyrazolo[1,5-a]pyrazine-2-carboxylate

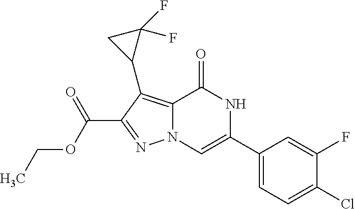 [SMILES: CCOC(=O)c1nn2cc(-c3ccc(Cl)c(F)c3)[nH]c(=O)c2c1C1CC1(F)F]

To a solution of diethyl 1-[2-(4-chloro-3-fluorophenyl)-2-oxoethyl]-4-(2,2-difluorocyclopropyl)-1H-pyrazole-3,5-dicarboxylate (Intermediate 379A, 383.0 mg, 0.8 mmol) in acetic acid (7 ml) was added ammonium acetate (643.4 mg, 8.3 mmol). The resulting mixture was heated overnight at 110° C. After cooled to RT, the reaction mixture was quenched with water. The solid was collected by filtration, washed with water and dried in vacuum to give 284.2 mg (83% of theory, 100% purity) of the title compound.

LC/MS [Method 3]: Rₜ=2.09 min; MS (ESIpos): m/z=410 [M−H]⁻.

### Intermediate 381A

### 6-(4-Chloro-3-fluorophenyl)-3-(2,2-difluorocyclopropyl)-4-oxo-4,5-dihydropyrazolo[1,5-a]pyrazine-2-carboxylic acid

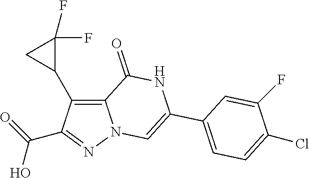 [SMILES: O=C(O)c1nn2cc(-c3ccc(Cl)c(F)c3)[nH]c(=O)c2c1C1CC1(F)F]

To a solution of ethyl 6-(4-chloro-3-fluorophenyl)-3-(2,2-difluorocyclopropyl)-4-oxo-4,5-dihydropyrazolo[1,5-a]pyrazine-2-carboxylate (Intermediate 380A, 284.2 mg, 0.69 mmol) in ethanol (5 ml) was added a solution 1N of lithium hydroxide (3.45 ml, 3.45 mmol). After stirring overnight at RT, the reaction mixture was concentrated under reduced pressure to remove ethanol. Then the rest of mixture was acidified with 1N hydrochloric acid. The product was collected by filtration, washed with water and dried in air to give 232.0 mg (86% of theory, 98% purity) of the title compound.

LC/MS [Method 3]: Rt =1.52 min; MS (ESIpos): m/z=382 [M−H]⁻.

¹H-NMR (400 MHz, DMSO-d₆): δ [ppm]=11.32 (bs, 1H), 11.70 (bs, 1H), 8.23 (s, 1H), 7.87 (dd, 1H), 7.73 (m, 1H), 7.65 (m, 1H), 3.14-3.05 (m, 1H), 2.16-2.04 (m, 2H).

### Intermediate 382A

Ethyl (2Z)-3-cyclobutyl-3-{[(trifluoromethyl)sulfonyl]oxy}acrylate

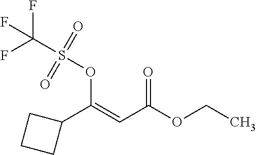 [SMILES: CCOC(=O)/C=C(\OS(=O)(=O)C(F)(F)F)C1CCC1]

To a stirred solution of ethyl 3-cyclobutyl-3-oxopropanoate (1.8 g, 10.6 mmol) in toluene (66 ml) was added a solution of lithium hydroxide (1.9 g, 79.3 mmol) in water (20 ml) at 10° C. After stirring for 5 min, trifluoromethanesulfonic anhydride (3.6 ml, 21.1 mmol) was added dropwise. After stirring for 2 h at RT, the reaction mixture was quenched with water (60 ml) and extracted with ethyl acetate (3×60 ml). The organic phase was washed with brine (1×60 ml) and dried over anhydrous sodium sulfate. The solid was filtered off. The filtrate was concentrated under reduced pressure to give 2.4 g (71% of theory, 93% purity) of the title compound.

LC/MS [Method 3]: Rₜ=2.25 min; MS (ESIpos): m/z=303 [M+H]⁺.

### Intermediate 383A

### Diethyl 4-cyclobutyl-1H-pyrazole-3,5-dicarboxylate

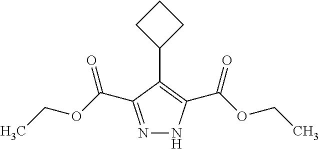 [SMILES: CCOC(=O)c1n[nH]c(C(=O)OCC)c1C1CCC1]

To a stirred solution of ethyl (2Z)-3-cyclobutyl-3-{[(trifluoromethyl)sulfonyl]oxy}acrylate (Intermediate 382A, 2.4 g, 8.06 mmol, 93% purity), ethyl diazoacetate (1.6 g, 12.1 mmol, 87% purity) and tetrakis(triphenylphosphine)palladium(0) in N,N-dimethylformamide (24 ml) was added 4-methylmorpholine (1.8 ml, 16.1 mmol) dropwise over 5 min. The resulting mixture was stirred overnight overnight at 60° C. The reaction mixture was quenched with water (40 ml) and extracted with ethyl acetate (3×40 ml). The organic phase was washed with brine and dried over anhydrous sodium sulfate. The solid was filtered off. The filtrate was concentrated under reduced pressure. The residue was purified by flash chromatography on silica gel (eluent: cyclohexane-ethyl acetate, 6:4) to give 1.28 g (60% of theory, 100% purity) of the title compound.

LC/MS [Method 3]: Rₜ=1.86 min; MS (ESIpos): m/z=267 [M+H]⁺.

¹H-NMR (400 MHz, DMSO-d₆): δ [ppm]=14.20 (bs, 1H), 4.38-4.24 (m, 5H), 2.48-2.40 (m, 2H), 2.19-2.12 (m, 2H), 1.99-1.90 (m, 1H), 1.87-1.78 (m, 1H), 1.36-1.29 (m, 6H).

### Intermediate 384A

### Diethyl 1-[2-(4-chloro-3-methylphenyl)-2-oxoethyl]-4-cyclobutyl-1H-pyrazole-3,5-dicarboxylate

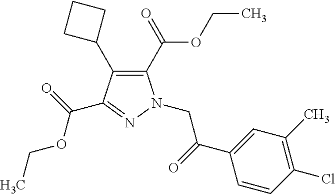 [SMILES: CCOC(=O)c1nn(CC(=O)c2ccc(Cl)c(C)c2)c(C(=O)OCC)c1C1CCC1]

To a solution of diethyl 4-cyclobutyl-1H-pyrazole-3,5-dicarboxylate (Intermediate 383A, 427 mg, 1.6 mmol) and 2-bromo-1-(4-chloro-3-methylphenyl)ethanone (501 mg, 1.9 mmol, 95% purity) in acetone (14 ml) was added potassium carbonate (554 mg, 4.0 mmol). The resulting mixture was stirred at RT for 1 h. After filtering off the solids, the filter cake was washed with acetone. The filtrate was concentrated under reduced pressure. The residue was purified by flash chromatography on silica gel (eluent: cyclohexane-ethyl acetate, 7:3) to give 708.0 mg (96% of theory, 94% purity) of the title compound.

LC/MS [Method 3]: Rₜ=2.53 min; MS (ESIpos): m/z=433 [M+H]⁺.

### Intermediate 385A

### Ethyl 6-(4-chloro-3-methylphenyl)-3-cyclobutyl-4-oxo-4,5-dihydropyrazolo[1,5-a]pyrazine-2-carboxylate

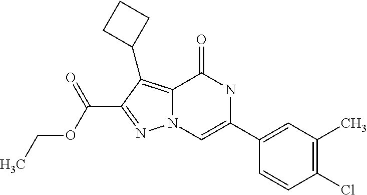 [SMILES: CCOC(=O)c1nn2cc(-c3ccc(Cl)c(C)c3)[nH]c(=O)c2c1C1CCC1]

To a solution of diethyl 1-[2-(4-chloro-3-methylphenyl)-2-oxoethyl]-4-cyclobutyl-1H-pyrazole-3,5-dicarboxylate (Intermediate 384A, 708.0 mg, 1.5 mmol, 94% purity) in acetic acid (13 ml) was added ammonium acetate (1.18 g, 15.4 mmol). The resulting mixture was heated for three days at 110° C. After cooling to RT, the reaction mixture was quenched with water. The solid was collected by filtration, washed with water and dried in vacuum to give 563.0 mg (82% of theory, 86% purity) of the title compound.

LC/MS [Method 3]: Rₜ=2.33 min; MS (ESIpos): m/z=386 [M+H]⁺.

### Intermediate 386A

### 6-(4-Chloro-3-methylphenyl)-3-cyclobutyl-4-oxo-4,5-dihydropyrazolo[1,5-a]pyrazine-2-carboxylic acid

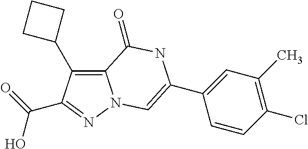 [SMILES: Cc1cc(-c2cn3nc(C(=O)O)c(C4CCC4)c3c(=O)[nH]2)ccc1Cl]

To a mixture of ethyl 6-(4-chloro-3-methylphenyl)-3-cyclobutyl-4-oxo-4,5-dihydropyrazolo[1,5-a]-pyrazine-2-carboxylate (Intermediate 385A, 562.0 mg, 1.25 mmol, 86% purity) in a mixture THF/methanol (13.6 ml, 5/1) was added a solution 1M of lithium hydroxide (6.3 ml). After stirring for two days at RT, the reaction mixture was concentrated under reduced pressure. Then the rest of mixture was diluted with water and acidified with 2N hydrochloric acid. The product was collected by filtration, washed with water and dried in high vacuum to give 464.0 mg (100% of theory, 100% purity) of the title compound.

LC/MS [Method 3]: Rₜ=1.83 min; MS (ESIpos): m/z=358 [M+H]⁺.

¹H-NMR (600 MHz, DMSO-d₆): δ [ppm]=11.53 (bs, 1H), 8.05 (s, 1H), 7.79 (d, 1H), 7.61 (dd, 1H), 7.52 (d, 1H), 4.61-4.55 (m, 1H), 2.73-2.67 (m, 2H), 2.51 (s, 3H), 2.19-2.15 (m, 2H), 1.99-1.92 (m, 1H), 1.90-1.85 (m, 1H).

### Intermediate 387A

### Diethyl 4-cyclobutyl-1-[2-(3,4-dichlorophenyl)-2-oxoethyl]-1H-pyrazole-3,5-dicarboxylate

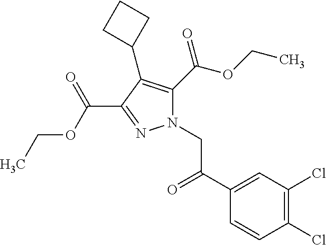 [SMILES: CCOC(=O)c1nn(CC(=O)c2ccc(Cl)c(Cl)c2)c(C(=O)OCC)c1C1CCC1]

To a solution of diethyl 4-cyclobutyl-1H-pyrazole-3,5-dicarboxylate (Intermediate 383A, 427 mg, 1.6 mmol) and 2-bromo-1-(3,4-dichlorophenyl)ethanone (515 mg, 1.9 mmol) in acetone (14 ml) was added potassium carbonate (554 mg, 4.0 mmol). The resulting mixture was stirred at RT for 1.5 h. After filtering off the solids, the filter cake was washed with acetone. The filtrate was concentrated under reduced pressure. The residue was purified by flash chromatography on silica gel (eluent: cyclohexane-ethyl acetate, 7:3) to give 778.0 mg (100% of theory, 96% purity) of the title compound.

LC/MS [Method 3]: Rₜ=2.54 min; MS (ESIpos): m/z=453 [M+H]⁺.

### Intermediate 388A

### Ethyl 3-cyclobutyl-6-(3,4-dichlorophenyl)-4-oxo-4,5-dihydropyrazolo[1,5-a]pyrazine-2-carboxylate

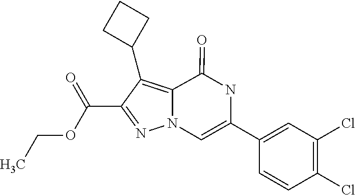 [SMILES: CCOC(=O)c1nn2cc(-c3ccc(Cl)c(Cl)c3)[nH]c(=O)c2c1C1CCC1]

To a solution of diethyl 4-cyclobutyl-1-[2-(3,4-dichlorophenyl)-2-oxoethyl]-1H-pyrazole-3,5-dicarboxylate (Intermediate 387A, 778 mg, 1.6 mmol, 96% purity) in acetic acid (14 ml) was added ammonium acetate (1.27 g, 16.5 mmol). The resulting mixture was heated overnight at 110° C. After cooling to RT, the reaction mixture was poured into ice-water. The solid was collected by filtration, washed with water and dried in vacuum to give 648.0 mg (92% of theory, 95% purity) of the title compound.

LC/MS [Method 3]: Rₜ=2.32 min; MS (ESIpos): m/z=406 [M+H]⁺.

### Intermediate 389A

### 3-Cyclobutyl-6-(3,4-dichlorophenyl)-4-oxo-4,5-dihydropyrazolo[1,5-a]pyrazine-2-carboxylic acid

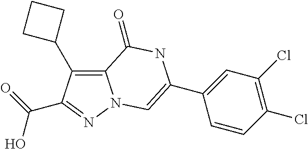 [SMILES: O=C(O)c1nn2cc(-c3ccc(Cl)c(Cl)c3)[nH]c(=O)c2c1C1CCC1]

To a mixture of ethyl 3-cyclobutyl-6-(3,4-dichlorophenyl)-4-oxo-4,5-dihydropyrazolo[1,5-a]pyrazine-2-carboxylate (Intermediate 388A, 645.0 mg, 1.5 mmol, 95% purity) in a mixture THF/methanol (16.4 ml, 5/1) was added a solution 1M of lithium hydroxide (7.5 ml). After stirring over two days at RT, the reaction mixture was concentrated under reduced pressure. Then the rest of mixture was diluted with water and acidified with 2N hydrochloric acid. The product was collected by filtration, washed with water and dried in high vacuum to give 542.0 mg (91% of theory, 96% purity) of the title compound.

LC/MS [Method 3]: Rₜ=1.83 min; MS (ESIpos): m/z=378 [M+H]⁺.

¹H-NMR (600 MHz, DMSO-d₆): δ [ppm]=11.62 (bs, 1H), 8.17 (s, 1H), 8.07 (s, 1H), 7.76 (s, 2H), 4.60-4.54 (m, 1H), 2.73-2.66 (m, 2H), 2.19-2.15 (m, 2H), 1.97-1.93 (m, 1H), 1.92-1.85 (m, 1H).

### Intermediate 390A

### Diethyl 1-[2-(4-chloro-3-fluorophenyl)-2-oxoethyl]-4-cyclobutyl-1H-pyrazole-3,5-dicarboxylate

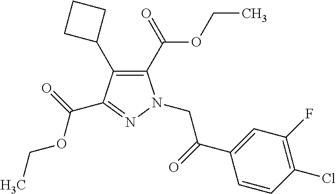 [SMILES: CCOC(=O)c1nn(CC(=O)c2ccc(Cl)c(F)c2)c(C(=O)OCC)c1C1CCC1]

To a solution of diethyl 4-cyclobutyl-1H-pyrazole-3,5-dicarboxylate (Intermediate 383A, 427 mg, 1.6 mmol) and 2-bromo-1-(4-chloro-3-fluorophenyl)ethanone (Intermediate 255A, 654 mg, 1.9 mmol, 74% purity) in acetone (14 ml) was added potassium carbonate (554 mg, 4.0 mmol). The resulting mixture was stirred at RT for 1 h. After filtering off the solids, the filter cake was washed with acetone. The filtrate was concentrated under reduced pressure. The residue was purified by flash chromatography on silica gel (eluent: cyclohexane-ethyl acetate, 7:3) to give 593 mg (63% of theory, 74% purity) of the title compound.

LC/MS [Method 3]: Rₜ=2.46 min; MS (ESIpos): m/z=437 [M+H]⁺.

### Intermediate 391A

### Ethyl 6-(4-chloro-3-fluorophenyl)-3-cyclobutyl-4-oxo-4,5-dihydropyrazolo[1,5-a]pyrazine-2-carboxylate

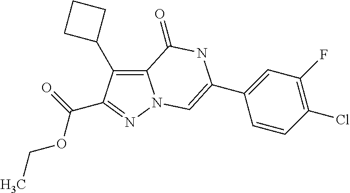 [SMILES: CCOC(=O)c1nn2cc(-c3ccc(Cl)c(F)c3)[nH]c(=O)c2c1C1CCC1]

To a solution of diethyl 1-[2-(4-chloro-3-fluorophenyl)-2-oxoethyl]-4-cyclobutyl-1H-pyrazole-3,5-dicarboxylate (Intermediate 390A, 593 mg, 1.4 mmol, 74% purity) in acetic acid (11 ml) was added ammonium acetate (1.05 g, 13.6 mmol). The resulting mixture was heated for two days at 110° C. After cooling to RT, the reaction mixture was quenched with water. The solid was collected by filtration, washed with water and dried in vacuum to give 502.0 mg (95% of theory, 100% purity) of the title compound.

LC/MS [Method 11]: Rₜ=1.15 min; MS (ESIpos): m/z=390 [M+H]⁺.

### Intermediate 392A

### 6-(4-Chloro-3-fluorophenyl)-3-cyclobutyl-4-oxo-4,5-dihydropyrazolo[1,5-a]pyrazine-2-carboxylic acid

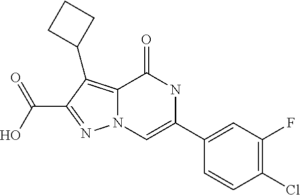 [SMILES: O=C(O)c1nn2cc(-c3ccc(Cl)c(F)c3)[nH]c(=O)c2c1C1CCC1]

To a mixture of ethyl 6-(4-chloro-3-fluorophenyl)-3-cyclobutyl-4-oxo-4,5-dihydropyrazolo[1,5-a]pyrazine-2-carboxylate (Intermediate 391A, 502 mg, 1.28 mmol) in a mixture THF/methanol (18 ml, 5/1) was added a solution 1M of lithium hydroxide (6.4 ml). After stirring overnight at RT, the reaction mixture was concentrated under reduced pressure. Then the rest of mixture was diluted with water and acidified with 2N hydrochloric acid. The product was collected by filtration, washed with water and dried in high vacuum to give 394.0 mg (85% of theory, 100% purity) of the title compound.

LC/MS [Method 3]: Rₜ=1.75 min; MS (ESIpos): m/z=362 [M+H]⁺.

¹H-NMR (400 MHz, DMSO-d₆): δ [ppm]=11.58 (bs, 1H), 8.17 (s, 1H), 7.89-7.86 (dd, 1H), 7.72 (t, 1H), 7.67-7.64 (dd, 1H), 4.61-4.52 (m, 1H), 2.74-2.64 (m, 2H), 2.20-2.13 (m, 2H), 2.00-1.84 (m, 2H)

### Intermediate 393A

### 2-Bromo-1-(3-fluoro-4-methylphenyl)ethanone

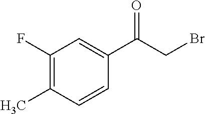

A solution of 1-(3-fluoro-4-methylphenyl)ethanone (10.0 g, 52.6 mmol, 80% purity) and phenyltrimethylammonium tribromide (24.4 g, 63.1 mmol, 97% purity) in THF (200 ml) was stirred for 1 h at 50° C. The ammonium salts were filtered off, and the filter cake was washed with THF. The filtrate was evaporated under reduced pressure and purified by flash chromatography on silica gel (eluent: cyclohexane-ethyl acetate 8:2) to give 10.9 g (73% of theory, 82% purity) of the title compound.

LC/MS [Method 3]: Rₜ=1.83 min; MS (ESIpos): m/z=230 [M+H]⁺.

### Intermediate 394A

### Diethyl 4-cyclobutyl-1-[2-(3-fluoro-4-methylphenyl)-2-oxoethyl]-1H-pyrazole-3,5-dicarboxylate

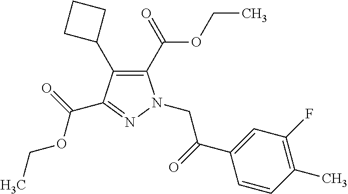 [SMILES: CCOC(=O)c1nn(CC(=O)c2ccc(C)c(F)c2)c(C(=O)OCC)c1C1CCC1]

To a solution of diethyl 4-cyclobutyl-1H-pyrazole-3,5-dicarboxylate (Intermediate 383A, 292.5 mg, 1.09 mmol) and 2-bromo-1-(3-fluoro-4-methylphenyl)ethanone (Intermediate 393A, 371.4 mg, 1.3 mmol, 82% purity) in acetone (10 ml) was added potassium carbonate (379 mg, 2.7 mmol). The resulting mixture was stirred at RT for 1 h. After filtering off the solids, the filter cake was washed with acetone. The filtrate was concentrated under reduced pressure to give 579.2 mg (91% of theory, 72% purity) of the title compound.

LC/MS [Method 3]: Rₜ=2.42 min; MS (ESIpos): m/z=417 [M+H]⁺.

### Intermediate 395A

### Ethyl 3-cyclobutyl-6-(3-fluoro-4-methylphenyl)-4-oxo-4,5-dihydropyrazolo[1,5-a]pyrazine-2-carboxylate

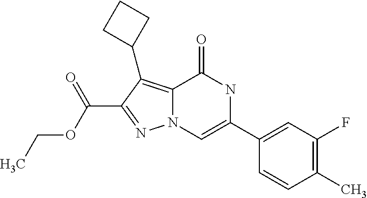 [SMILES: CCOC(=O)c1nn2cc(-c3ccc(C)c(F)c3)[nH]c(=O)c2c1C1CCC1]

To a solution of diethyl 4-cyclobutyl-1-[2-(3-fluoro-4-methylphenyl)-2-oxoethyl]-1H-pyrazole-3,5-dicarboxylate (Intermediate 394A, 579.2 mg, 1.27 mmol, 92% purity) in acetic acid (5.3 ml) was added ammonium acetate (983 mg, 12.7 mmol). The resulting mixture was heated for two days at 110° C. After cooling to RT, the reaction mixture was quenched with water. The solid was collected by filtration, washed with water and dried in vacuum to give 325.5 mg (62% of theory, 90% purity) of the title compound.

LC/MS [Method 3]: Rₜ=2.17 min; MS (ESIpos): m/z=370 [M+H]⁺.

### Intermediate 396A

### 3-Cyclobutyl-6-(3-fluoro-4-methylphenyl)-4-oxo-4,5-dihydropyrazolo[1,5-a]pyrazine-2-carboxylic acid

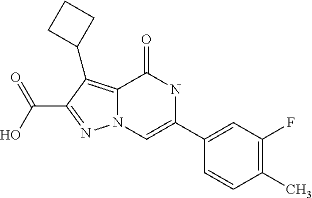 [SMILES: Cc1ccc(-c2cn3nc(C(=O)O)c(C4CCC4)c3c(=O)[nH]2)cc1F]

To a mixture of ethyl 3-cyclobutyl-6-(3-fluoro-4-methylphenyl)-4-oxo-4,5-dihydropyrazolo[1,5-a]-pyrazine-2-carboxylate (Intermediate 395A, 325.5 mg, 0.79 mmol, 90% purity) in a mixture THF/methanol (6 ml, 5/1) was added a solution 1M of lithium hydroxide (3.9 ml). After stirring for 6 h at RT, the reaction mixture was concentrated under reduced pressure. Then the rest of mixture was diluted with water and acidified with 2N hydrochloric acid. The product was collected by filtration, washed with water and dried in high vacuum to give 241.0 mg (89% of theory, 100% purity) of the title compound.

LC/MS [Method 3]: Rₜ=1.67 min; MS (ESIpos): m/z=342 [M+H]⁺.

### Intermediate 397A

### Diethyl 4-cyclobutyl-1-[2-(3,4-dimethylphenyl)-2-oxoethyl]-1H-pyrazole-3,5-dicarboxylate

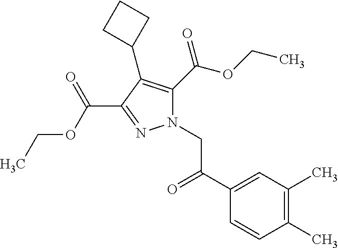 [SMILES: CCOC(=O)c1nn(CC(=O)c2ccc(C)c(C)c2)c(C(=O)OCC)c1C1CCC1]

To a solution of diethyl 4-cyclobutyl-1H-pyrazole-3,5-dicarboxylate (Intermediate 383A, 292.5 mg, 1.09 mmol) and 2-bromo-1-(3,4-dimethylphenyl)ethanone (Intermediate 20A, 299.3 mg, 1.3 mmol) in acetone (9.6 ml) was added potassium carbonate (379 mg, 2.7 mmol). The resulting mixture was stirred at RT for 1 h. After filtering off the solids, the filter cake was washed with acetone. The filtrate was concentrated under reduced pressure to give 543.0 mg (83% of theory, 69% purity) of the title compound.

LC/MS [Method 3]: Rₜ=2.47 min; MS (ESIpos): m/z=413 [M+H]⁺.

### Intermediate 398A

### Ethyl 3-cyclobutyl-6-(3,4-dimethylphenyl)-4-oxo-4,5-dihydropyrazolo[1,5-a]pyrazine-2-carboxylate

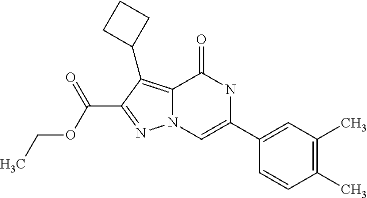 [SMILES: CCOC(=O)c1nn2cc(-c3ccc(C)c(C)c3)[nH]c(=O)c2c1C1CCC1]

To a solution of diethyl 4-cyclobutyl-1-[2-(3,4-dimethylphenyl)-2-oxoethyl]-1H-pyrazole-3,5-dicarboxylate (Intermediate 398A, 543.0 mg, 0.9 mmol, 69% purity) in acetic acid (3.8 ml) was added ammonium acetate (703 mg, 9.1 mmol). The resulting mixture was heated for two days at 110° C. After cooling to RT, the reaction mixture was quenched with water. The solid was collected by filtration, washed with water and dried in vacuum to give 166.0 mg (50% of theory, 100% purity) of the title compound.

LC/MS [Method 3]: Rₜ=2.26 min; MS (ESIpos): m/z=366 [M+H]⁺.

### Intermediate 399A

### 3-Cyclobutyl-6-(3,4-dimethylphenyl)-4-oxo-4,5-dihydropyrazolo[1,5-a]pyrazine-2-carboxylic acid

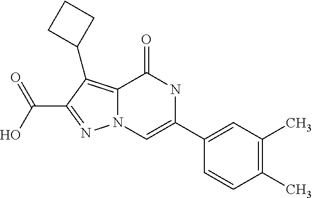 [SMILES: Cc1ccc(-c2cn3nc(C(=O)O)c(C4CCC4)c3c(=O)[nH]2)cc1C]

To a mixture of ethyl ethyl 3-cyclobutyl-6-(3,4-dimethylphenyl)-4-oxo-4,5-dihydropyrazolo[1,5-a]-pyrazine-2-carboxylate (Intermediate 398A, 166.0 mg, 0.45 mmol) in a mixture THF/methanol (3.4 ml, 5/1) was added a solution 1M of lithium hydroxide (2.3 ml). After stirring for 6 h at RT, the reaction mixture was concentrated under reduced pressure. Then the rest of mixture was diluted with water and acidified with 3N hydrochloric acid. The product was collected by filtration, washed with water and dried in high vacuum to give 129.8 mg (85% of theory, 100% purity) of the title compound.

LC/MS [Method 3]: Rₜ=1.75 min; MS (ESIpos): m/z=338 [M+H]⁺.

### Intermediate 400A

### 2-Bromo-1-(5-chloro-6-methylpyridin-3-yl)ethanone

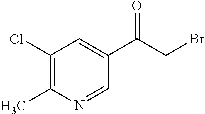

A solution of 1-(5-chloro-6-methylpyridin-3-yl)ethanone (2.0 g, 11.8 mmol) and phenyltrimethylammonium tribromide (4.4 g, 11.8 mmol) in THF (40 ml) was stirred for 3 h at 50° C. The ammonium salts were filtered off, and the filter cake was washed with acetonitrile. The filtrate was evaporated under reduced pressure to give 6.6 g (81% of theory, 48% purity) of the title compound which was used in the next step without further purification.

LC/MS [Method 3]: Rₜ=1.60 min; MS (ESIpos): m/z=247 [M+H]⁺.

### Intermediate 401A

### Diethyl 1-[2-(5-chloro-6-methylpyridin-3-yl)-2-oxoethyl]-4-cyclopropyl-1H-pyrazole-3,5-dicarboxylate

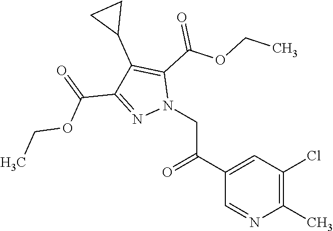 [SMILES: CCOC(=O)c1nn(CC(=O)c2cnc(C)c(Cl)c2)c(C(=O)OCC)c1C1CC1]

To a solution of diethyl 4-cyclopropyl-1H-pyrazole-3,5-dicarboxylate (Intermediate 187A, 1.1 g, 3.9 mmol, 92% purity) and 2-bromo-1-(5-chloro-6-methylpyridin-3-yl)ethanone (Intermediate 400A, 1.6 g, 4.8 mmol, 48% purity) in acetone (35 ml) was added potassium carbonate (1.9 g, 13.9 mmol). The resulting mixture was stirred at RT overnight. After filtering off the solids, the filter cake was washed with acetone. The filtrate was evaporated under reduced pressure and purified by flash chromatography on silica gel (eluent: cyclohexane-ethyl acetate 7/3) to give 791.0 mg (47% of theory, 100% purity) of the title compound.

LC/MS [Method 3]: Rₜ=2.15 min; MS (ESIpos): m/z=420 [M+H]⁺.

### Intermediate 402A

### Ethyl 6-(5-chloro-6-methylpyridin-3-yl)-3-cyclopropyl-4-oxo-4,5-dihydropyrazolo[1,5-a]pyrazine-2-carboxylate

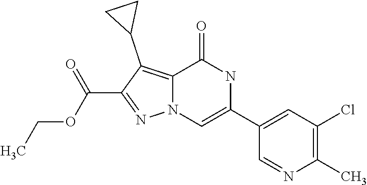

To a solution of diethyl 1-[2-(5-chloro-6-methylpyridin-3-yl)-2-oxoethyl]-4-cyclopropyl-1H-pyrazole-3,5-dicarboxylate (Intermediate 401A, 791.0 mg, 1.9 mmol) in acetic acid (15.8 ml) was added ammonium acetate (1.4 g, 18.8 mmol). The resulting mixture was heated overnight at 110° C. After cooling to RT, the reaction mixture was quenched with water. The solid was collected by filtration, washed with water and dried in vacuum to give 600.0 mg (85% of theory, 100% purity) of the title compound.

LC/MS [Method 3]: Rₜ=1.80 min; MS (ESIpos): m/z=373 [M+H]⁺.

¹H-NMR (400 MHz, DMSO-d₆): δ [ppm]=11.63 (bs, 1H), 8.78 (d, 1H), 8.28 (d, 1H), 8.26 (d, 1H), 4.36-4.31 (q, 2H), 2.69-2.62 (m, 1H), 2.60 (s, 3H), 1.33 (t, 3H), 1.23-1.19 (m, 2H), 0.98-0.93 (m, 2H).

### Intermediate 403A

### 6-(5-Chloro-6-methylpyridin-3-yl)-3-cyclopropyl-4-oxo-4,5-dihydropyrazolo[1,5-a]pyrazine-2-carboxylic acid

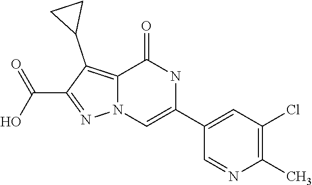

To a mixture of ethyl 6-(5-chloro-6-methylpyridin-3-yl)-3-cyclopropyl-4-oxo-4,5-dihydropyrazolo-[1,5-a]pyrazine-2-carboxylate (Intermediate 402A, 600.0 mg, 1.6 mmol) in a mixture THF/methanol (18 ml, 5/1) was added a solution 1M of lithium hydroxide (8.0 ml). After stirring overnight at RT, the reaction mixture was concentrated under reduced pressure. Then the rest of mixture was diluted with water and acidified with 2N hydrochloric acid. The product was collected by filtration, washed with water and dried in high vacuum to give 678.0 mg (100% of theory, 100% purity) of the title compound.

LC/MS [Method 11]: Rₜ=0.74 min; MS (ESIpos): m/z=345 [M+H]⁺.

¹H-NMR (400 MHz, DMSO-d₆): δ [ppm]=13.14 (bs, 1H), 11.59 (s, 1H), 8.79 (d, 1H), 8.27 (d, 1H), 8.22 (d, 1H), 2.76-2.70 (m, 1H), 2.60 (s, 3H), 1.26-1.26 (m, 2H), 0.95-0.91 (m, 2H).

### Intermediate 404A

### 2-Bromo-1-(4,5-dichloro-2-fluorophenyl)ethanone

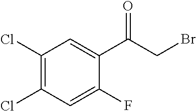 [SMILES: O=C(CBr)c1cc(Cl)c(Cl)cc1F]

A solution of 1-(4,5-dichloro-2-fluorophenyl)ethanone (1.0 g, 4.8 mmol) and phenyltrimethylammonium tribromide (1.8 g, 4.8 mmol) in THF (20 ml) was stirred for 2 h at 50° C. The ammonium salts were filtered off, and the filter cake was washed with THF. The filtrate was evaporated under reduced pressure to give 1.3 g (75% of theory, 77% purity) of the title compound which was used in the next step without further purification.

GC/MS [Method 53]: Rₜ=5.82 min; MS (ESIpos): m/z=286 [M+H]⁺.

### Intermediate 405A

### Diethyl 4-cyclopropyl-1-[2-(4,5-dichloro-2-fluorophenyl)-2-oxoethyl]-1H-pyrazole-3,5-dicarboxylate

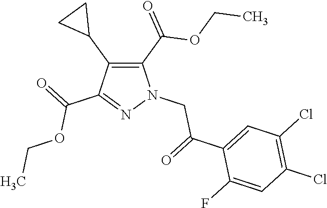 [SMILES: CCOC(=O)c1nn(CC(=O)c2cc(Cl)c(Cl)cc2F)c(C(=O)OCC)c1C1CC1]

To a solution of diethyl 4-cyclopropyl-1H-pyrazole-3,5-dicarboxylate (Intermediate 187A, 500.0 mg, 1.9 mmol) and 2-bromo-1-(4,5-dichloro-2-fluorophenyl)ethanone (Intermediate 404A, 802.9 mg, 2.4 mmol, 77% purity) in acetone (17 ml) was added potassium carbonate (684.8 mg, 4.9 mmol). The resulting mixture was stirred at RT for 1 h. After filtering off the solids, the filter cake was washed with acetone. The filtrate was evaporated under reduced pressure to give 1.38 g (100% of theory, 92% purity) of the title compound.

LC/MS [Method 3]: Rₜ=2.45 min; MS (ESIpos): m/z=457 [M+H]⁺.

### Intermediate 406A

### Ethyl 3-cyclopropyl-6-(4,5-dichloro-2-fluorophenyl)-4-oxo-4,5-dihydropyrazolo[1,5-a]pyrazine-2-carboxylate

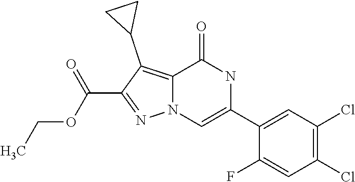

To a solution of diethyl 4-cyclopropyl-1-[2-(4,5-dichloro-2-fluorophenyl)-2-oxoethyl]-1H-pyrazole-3,5-dicarboxylate (Intermediate 405A, 1.38 g, 2.8 mmol, 92% purity) in acetic acid (11.6 ml) was added ammonium acetate (2.1 g, 27.6 mmol). The resulting mixture was heated for 7 days at 110° C. After cooling to RT, the reaction mixture was quenched with water. The solid was collected by filtration, washed with MTBE and dried in vacuum to give 537.0 mg (47% of theory, 100% purity) of the title compound.

LC/MS [Method 3]: Rₜ=2.14 min; MS (ESIpos): m/z=410 [M+H]⁺.

### Intermediate 407A

### 3-Cyclopropyl-6-(4,5-dichloro-2-fluorophenyl)-4-oxo-4,5-dihydropyrazolo[1 ,5-a]pyrazine-2-carboxylic acid

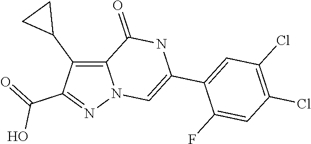

To a mixture of ethyl 3-cyclopropyl-6-(4,5-dichloro-2-fluorophenyl)-4-oxo-4,5-dihydropyrazolo-[1,5-a]pyrazine-2-carboxylate (Intermediate 406A, 537.0 mg, 1.3 mmol) in a mixture THF/methanol (10 ml, 5/1) was added a solution 1M of lithium hydroxide (6.5 ml). After stirring overnight at RT, the reaction mixture was concentrated under reduced pressure. Then the rest of mixture was diluted with water and acidified with 1N hydrochloric acid. The product was collected by filtration, washed with water and dried in high vacuum to give 498.0 mg (98% of theory, 98% purity) of the title compound.

LC/MS [Method 11]: Rₜ=1.62 min; MS (ESIpos): m/z=380 [M+H]⁺.

¹H-NMR (600 MHz, DMSO-d₆): δ [ppm]=11.56 (bs, 1H), 7.97 (d, 1H), 7.91 (m, 2H), 2.74-2.70 (m, 1H), 1.25-1.22 (m, 2H), 0.94-0.91 (m, 2H).

### Intermediate 408A

### 2-Bromo-1-(4-chloro-2,5-difluorophenyl)ethanone

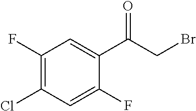 [SMILES: O=C(CBr)c1cc(F)c(Cl)cc1F]

A solution of 1-(4-chloro-2,5-difluorophenyl)ethanone (1.0 g, 5.2 mmol) and phenyltrimethylammonium tribromide (2.4 g, 6.3 mmol) in THF (18 ml) was stirred for 1 h at 50° C. The ammonium salts were filtered off, and the filter cake was washed with THF. The filtrate was evaporated under reduced pressure to give 2.4 g (>100% of theory, 64% purity) of the title compound which was used in the next step without further purification.

### Intermediate 409A

### Diethyl 1-[2-(4-chloro-2,5-difluorophenyl)-2-oxoethyl]-4-cyclopropyl-1H-pyrazole-3,5-dicarboxylate

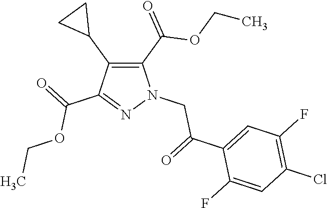 [SMILES: CCOC(=O)c1nn(CC(=O)c2cc(F)c(Cl)cc2F)c(C(=O)OCC)c1C1CC1]

To a solution of diethyl 4-cyclopropyl-1H-pyrazole-3,5-dicarboxylate (Intermediate 187A, 1.1 g, 4.2 mmol) and 2-bromo-1-(4-chloro-2,5-difluorophenyl)ethanone (Intermediate 408A, 2.1 g, 5.1 mmol, 64% purity) in acetone (38 ml) was added potassium carbonate (1.47 g, 10.6 mmol). The resulting mixture was stirred at RT for 1.5 h. After filtering off the solids, the filter cake was washed with acetone. The filtrate was evaporated under reduced pressure to give 2.7 g (100% of theory, 72% purity) of the title compound.

LC/MS [Method 3]: Rₜ=2.36 min; MS (ESIpos): m/z=441 [M+H]⁺.

### Intermediate 410A

### Ethyl 6-(4-chloro-2,5-difluorophenyl)-3-cyclopropyl-4-oxo-4,5-dihydropyrazolo[1,5-a]pyrazine-2-carboxylate

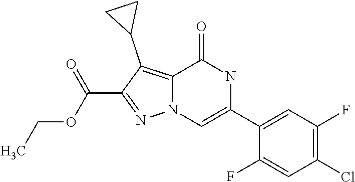

To a solution of diethyl 1-[2-(4-chloro-2,5-difluorophenyl)-2-oxoethyl]-4-cyclopropyl-1H-pyrazole-3,5-dicarboxylate (Intermediate 409A, 3.0 g, 4.9 mmol, 72% purity) in acetic acid (30 ml) was added ammonium acetate (3.8 g, 48.7 mmol). The resulting mixture was heated overnight at 110° C. After cooling to RT, the reaction mixture was quenched with water. The solid was collected by filtration, washed with water and dried in vacuum to give 1.2 g (54% of theory, 86% purity) of the title compound.

LC/MS [Method 11]: Rₜ=1.05 min; MS (ESIpos): m/z=394 [M+H]⁺.

### Intermediate 411A

### 6-(4-Chloro-2,5-difluorophenyl)-3-cyclopropyl-4-oxo-4,5-dihydropyrazolo[1,5-a]pyrazine-2-carboxylic acid

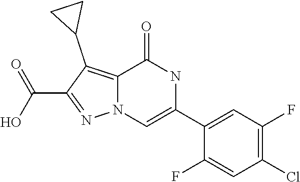

To a mixture of ethyl 6-(4-chloro-2,5-difluorophenyl)-3-cyclopropyl-4-oxo-4,5-dihydropyrazolo-[1,5-a]pyrazine-2-carboxylate (Intermediate 410A, 1.2 g, 3.1 mmol) in a mixture THF/methanol (23 ml, 5/1) was added a solution 1M of lithium hydroxide (15.3 ml). After stirring overnight at RT, the reaction mixture was concentrated under reduced pressure. Then the rest of mixture was diluted with water and acidified with 1N hydrochloric acid. The product was collected by filtration, washed with water and dried in high vacuum to give 917.0 mg (82% of theory, 100% purity) of the title compound.

LC/MS [Method 3]: Rₜ=1.52 min; MS (ESIpos): m/z=364 [M−H]⁻.

### Intermediate 412A

### 2-Bromo-1-(3-fluoro-4-methoxphenyl)ethanone

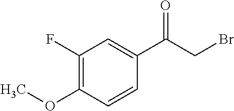

A solution of 1-(3-fluoro-4-methoxyphenyl)ethanone (5.0 g, 29.7 mmol) and phenyltrimethylammonium tribromide (11.2 g, 29.7 mmol) in THF (110 ml) was stirred for 2 h at 50° C. The ammonium salts were filtered off, and the filter cake was washed with THF. The filtrate was evaporated under reduced pressure to give 11.2 g (89% of theory, 58% purity) of the title compound which was used in the next step without further purification.

GC/MS [Method 53]: Rₜ=6.06 min; MS (ESIpos): m/z=247 [M+H]⁺.

### Intermediate 413A

### Diethyl 4-cyclopropyl-1-[2-(3-fluoro-4-methoxyphenyl)-2-oxoethyl]-1H-pyrazole-3,5-dicarboxylate

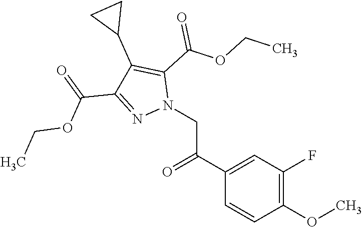 [SMILES: CCOC(=O)c1nn(CC(=O)c2ccc(OC)c(F)c2)c(C(=O)OCC)c1C1CC1]

To a solution of diethyl 4-cyclopropyl-1H-pyrazole-3,5-dicarboxylate (Intermediate 187A, 2.0 g, 7.9 mmol) and 2-bromo-1-(3-fluoro-4-methoxyphenyl)ethanone (Intermediate 412A, 3.7 g, 8.7 mmol, 58% purity) in acetone (70 ml) was added potassium carbonate (2.7 g, 19.8 mmol). The resulting mixture was stirred at RT for 1 h. After filtering off the solids, the filter cake was washed with acetone. The filtrate was evaporated under reduced pressure and purified by flash chromatography on silica gel (eluent: cyclohexane-ethyl acetate 6/4) to give 3.25 g (98% of theory) of the title compound.

LC/MS [Method 3]: Rₜ=2.11 min; MS (ESIpos): m/z=419 [M+H]⁺.

### Intermediate 414A

### Ethyl 3-cyclopropyl-6-(3-fluoro-4-methoxphenyl)-4-oxo-4,5-dihydropyrazolo[1,5-a]pyrazine-2-carboxylate

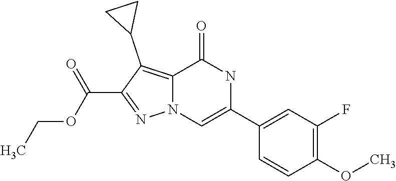

To a solution of diethyl 4-cyclopropyl-1-[2-(3-fluoro-4-methoxphenyl)-2-oxoethyl]-1H-pyrazole-3,5-dicarboxylate (Intermediate 413A, 3.25 g, 7.8 mmol) in acetic acid (51 ml) was added ammonium acetate (5.9 g, 77.7 mmol). The resulting mixture was heated for 48 h at 110° C. After cooling to RT, the reaction mixture was quenched with water. The solid was collected by filtration, washed with water and dried in vacuum to give 2.85 g (83% of theory, 84% purity) of the title compound. LC/MS [Method 3]: Rₜ=1.86 min; MS (ESIpos): m/z=372 [M+H]⁺.

### Intermediate 415A

### 3-Cyclopropyl-6-(3-fluoro-4-methoxyphenyl)-4-oxo-4,5-dihydropyrazolo[1 ,5-a]pyrazine-2-carboxylic acid

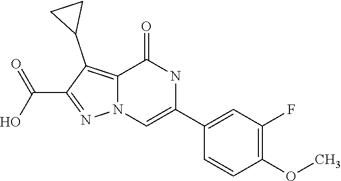 [SMILES: COc1ccc(-c2cn3nc(C(=O)O)c(C4CC4)c3c(=O)[nH]2)cc1F]

To a mixture of ethyl 3-cyclopropyl-6-(3-fluoro-4-methoxphenyl)-4-oxo-4,5-dihydropyrazolo-[1,5-a]pyrazine-2-carboxylate (Intermediate 414A, 2.85 g, 7.7 mmol) in a mixture THF/methanol (55.6 ml, 5/1) was added a solution 1M of lithium hydroxide (38.4 ml). After stirring overnight at RT, the reaction mixture was concentrated under reduced pressure. Then the rest of mixture was diluted with water and acidified with 3N hydrochloric acid. The product was collected by filtration, washed with water and dried in high vacuum to give 1.99 g (76% of theory, 100% purity) of the title compound. LC/MS [Method 3]: Rₜ=1.36 min; MS (ESIpos): m/z=342 [M−H]⁻.

### Intermediate 416A

### Diethyl 4-cyclopropyl-1-{2-[3-fluoro-4-(trifluoromethyl)phenyl]-2-oxoethyl}-1H-pyrazole-3,5-dicarboxylate

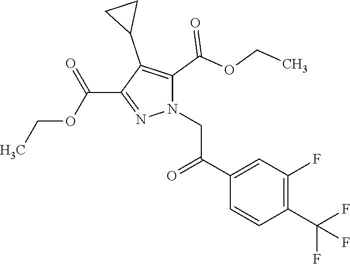 [SMILES: CCOC(=O)c1nn(CC(=O)c2ccc(C(F)(F)F)c(F)c2)c(C(=O)OCC)c1C1CC1]

To a solution of diethyl 4-cyclopropyl-1H-pyrazole-3,5-dicarboxylate (Intermediate 187A, 1.0 g, 3.9 mmol) and 2-bromo-1-[3-fluoro-4-(trifluoromethyl)phenyl]ethanone (3.7 g, 4.8 mmol, 50% purity) in acetone (35 ml) was added potassium carbonate (1.37 g, 9.9 mmol). The resulting mixture was stirred at RT for 2 h. After filtering off the solids, the filter cake was washed with acetone. The filtrate was evaporated under reduced pressure to give 3.28 g (>100% of theory, 74% purity) of the title compound.

LC/MS [Method 3]: Rₜ=2.35 min; MS (ESIpos): m/z=457 [M+H]⁺.

### Intermediate 417A

### Ethyl 3-cyclopropyl-6-[3-fluoro-4-(trifluoromethyl)phenyl]-4-oxo-4,5-dihydropyrazolo[1,5-a]pyrazine-2-carboxylate

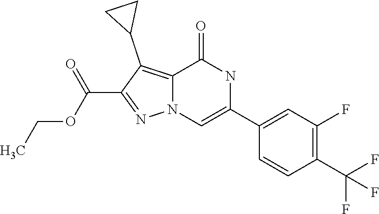 [SMILES: CCOC(=O)c1nn2cc(-c3ccc(C(F)(F)F)c(F)c3)[nH]c(=O)c2c1C1CC1]

To a solution of diethyl 4-cyclopropyl-1-{2-[3-fluoro-4-(trifluoromethyl)phenyl]-2-oxoethyl}-1H-pyrazole-3,5-dicarboxylate (Intermediate 416A, 3.28 g, 5.3 mmol, 74% purity) in acetic acid (45 ml) was added ammonium acetate (4.1 g, 53.3 mmol). The resulting mixture was heated for 48 h at 110° C. After cooling to RT, the reaction mixture was quenched with water. The solid was collected by filtration, washed with water and dried in vacuum to give 1.77 g (71% of theory, 88% purity) of the title compound.

LC/MS [Method 3]: Rₜ=2.07 min; MS (ESIpos): m/z=410 [M+H]⁺.

### Intermediate 418A

### 3-Cyclopropyl-6-[3-fluoro-4-(trifluoromethyl)phenyl]-4-oxo-4,5-dihydropyrazolo[1 ,5-a]pyrazine-2-carboxylic acid

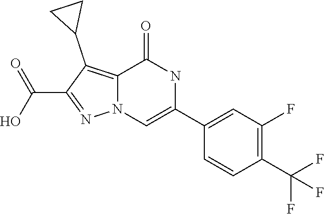 [SMILES: O=C(O)c1nn2cc(-c3ccc(C(F)(F)F)c(F)c3)[nH]c(=O)c2c1C1CC1]

To a mixture of ethyl 3-cyclopropyl-6-[3-fluoro-4-(trifluoromethyl)phenyl]-4-oxo-4,5-dihydropyrazolo[1,5-a]pyrazine-2-carboxylate (Intermediate 417A, 1.77 g, 4.3 mmol, 88% purity) in a mixture THF/methanol (34.6 ml, 5/1) was added a solution 1M of lithium hydroxide (21.6 ml). After stirring for 48 h at RT, the reaction mixture was concentrated under reduced pressure. Then the rest of mixture was diluted with water and acidified with 4N hydrochloric acid. The product was collected by filtration, washed with water and dried in high vacuum to give 1.29 g (78% of theory, 100% purity) of the title compound.

LC/MS [Method 3]: Rₜ=1.64 min; MS (ESIpos): m/z=382 [M+H]⁺.

### Intermediate 419A

### 2-Bromo-1-(5-chloro-6-methylpyridin-2-yl)ethanone

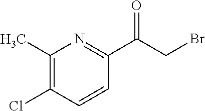

A solution of 1-(5-chloro-6-methylpyridin-2-yl)ethanone (1.0 g, 5.9 mmol) and phenyltrimethylammonium tribromide (2.2 g, 5.9 mmol) in THF (20 ml) was stirred for 2 h at 50° C. The ammonium salts were filtered off, and the filter cake was washed with acetonitrile. The filtrate was evaporated under reduced pressure to give 3.2 g (>100% of theory, 76% purity) of the title compound which was used in the next step without further purification.

LC/MS [Method 3]: Rₜ=1.99 min; MS (ESIpos): m/z=247 [M+H]⁺.

### Intermediate 420A

### Diethyl 1-[2-(5-chloro-6-methylpyridin-2-yl)-2-oxoethyl]-4-cyclopropyl-1H-pyrazole-3,5-dicarboxylate

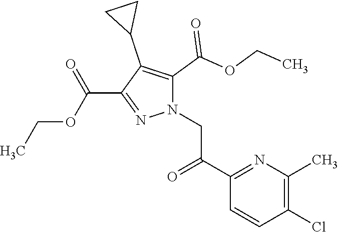 [SMILES: CCOC(=O)c1nn(CC(=O)c2ccc(Cl)c(C)n2)c(C(=O)OCC)c1C1CC1]

To a solution of diethyl 4-cyclopropyl-1H-pyrazole-3,5-dicarboxylate (Intermediate 187A, 344 mg, 1.37 mmol) and 2-bromo-1-(5-chloro-6-methylpyridin-2-yl)ethanone (Intermediate 419A, 540 mg, 1.6 mmol, 76% purity) in acetone (12 ml) was added potassium carbonate (661 mg, 4.8 mmol). The resulting mixture was stirred at RT for 48 h. After filtering off the solids, the filter cake was washed with acetone. The filtrate was evaporated under reduced pressure and purified by flash chromatography on silica gel (eluent: cyclohexane-ethyl acetate 6/4) to give 623.9 mg (73% of theory, 68% purity) of the title compound.

LC/MS [Method 3]: Rₜ=2.13 min; MS (ESIpos): m/z=420 [M+H]⁺.

### Intermediate 421A

### Ethyl 6-(5-chloro-6-methylpyridin-2-yl)-3-cyclopropyl-4-oxo-4,5-dihydropyrazolo[1,5-a]pyrazine-2-carboxylate

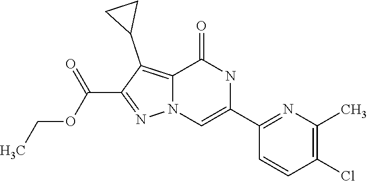 [SMILES: CCOC(=O)c1nn2cc(-c3ccc(Cl)c(C)n3)[nH]c(=O)c2c1C1CC1]

To a solution of diethyl 1-[2-(5-chloro-6-methylpyridin-2-yl)-2-oxoethyl]-4-cyclopropyl-1H-pyrazole-3,5-dicarboxylate (Intermediate 420A, 623.9 mg, 1.0 mmol, 68% purity) in acetic acid (8.4 ml) was added ammonium acetate (773.1 mg, 10.0 mmol). The resulting mixture was heated for 48 h at 110° C. After cooling to RT, the reaction mixture was quenched with water. The solid was collected by filtration, washed with water and dried in vacuum to give 376.7 mg (80% of theory, 80% purity) of the title compound.

LC/MS [Method 3]: Rₜ=1.92 min; MS (ESIpos): m/z=373 [M+H]⁺.

### Intermediate 422A

### 6-(5-Chloro-6-methylpyridin-2-yl)-3-cyclopropyl-4-oxo-4,5-dihydropyrazolo[1,5-a]pyrazine-2-carboxylic acid

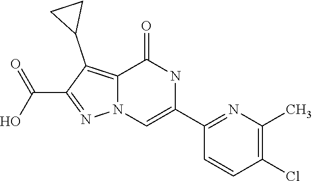 [SMILES: Cc1nc(-c2cn3nc(C(=O)O)c(C4CC4)c3c(=O)[nH]2)ccc1Cl]

To a mixture of ethyl 6-(5-chloro-6-methylpyridin-2-yl)-3-cyclopropyl-4-oxo-4,5-dihydropyrazolo-[1,5-a]pyrazine-2-carboxylate (Intermediate 421A, 367.7 mg, 0.79 mmol, 80% purity) in a mixture THF/methanol (5.7 ml, 5/1) was added a solution 1M of lithium hydroxide (3.9 ml). After stirring overnight at RT, the reaction mixture was concentrated under reduced pressure. Then the rest of mixture was diluted with water and acidified with 1N hydrochloric acid. The product was collected by filtration, washed with water and dried in high vacuum to give 289.0 mg (99% of theory, 93% purity) of the title compound.

LC/MS [Method 3]: Rₜ=1.27 min; MS (ESIpos): m/z=345 [M−H]⁻.

### Intermediate 423A

### 2-Bromo-1-[4-chloro-3-(trifluoromethoxy)phenyl]ethanone

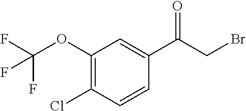 [SMILES: O=C(CBr)c1ccc(Cl)c(OC(F)(F)F)c1]

A solution of 1-[4-chloro-3-(trifluoromethoxy)phenyl]ethanone (2.0 g, 8.4 mmol) and phenyltrimethylammonium tribromide (3.2 g, 8.3 mmol) in THF (40 ml) was stirred for 2 h at 50° C. The ammonium salts were filtered off, and the filter cake was washed with THF. The filtrate was evaporated under reduced pressure to give 3.7 g (>100% of theory, 76% purity) of the title compound which was used in the next step without further purification.

LC/MS [Method 11]: Rₜ=1.13 min; MS (ESIpos): m/z=316 [M−H]⁻.

### Intermediate 424A

### Diethyl 1-{2-[4-chloro-3-(trifluoromethoxy)phenyl]-2-oxoethyl}-4-cyclopropyl-1H-pyrazole-3,5-dicarboxylate

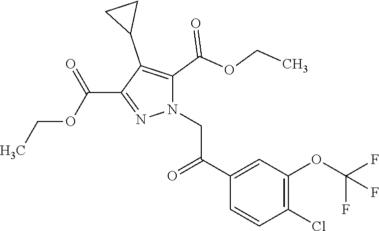 [SMILES: CCOC(=O)c1nn(CC(=O)c2ccc(Cl)c(OC(F)(F)F)c2)c(C(=O)OCC)c1C1CC1]

To a solution of diethyl 4-cyclopropyl-1H-pyrazole-3,5-dicarboxylate (Intermediate 187A, 2.0 g, 7.9 mmol) and 2-bromo-1-[4-chloro-3-(trifluoromethoxy)phenyl]ethanone (Intermediate 423A, 2.7 g, 8.7 mmol, 76% purity) in acetone (70 ml) was added potassium carbonate (2.7 g, 19.8 mmol). The resulting mixture was stirred at RT for 1.5 h. At this point in time, the LC/MS showed no full conversion to the product, and additional 2-bromo-1-[4-chloro-3-(trifluoromethoxy)phenyl]ethanone (Intermediate 423A, 800.0 mg, 76% purity) was added. The reaction was stirred at RT overnight. After filtering off the solids, the filter cake was washed with acetone. The filtrate was evaporated under reduced pressure and purified by flash chromatography on silica gel (eluent: cyclohexane-ethyl acetate 7/3) to give 2.6 g (65% of theory, 95% purity) of the title compound. LC/MS [Method 3]: Rₜ=2.44 min; MS (ESIpos): m/z=489 [M+H]⁺.

### Intermediate 425A

### Ethyl 6-[4-chloro-3-(trifluoromethoxy)phenyl]-3-cyclopropyl-4-oxo-4,5-dihydropyrazolo[1,5-a]-pyrazine-2-carboxylate

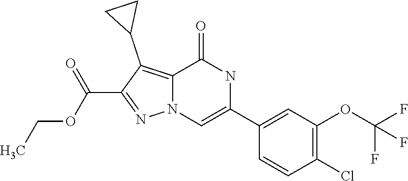 [SMILES: CCOC(=O)c1nn2cc(-c3ccc(Cl)c(OC(F)(F)F)c3)[nH]c(=O)c2c1C1CC1]

To a solution of diethyl 1-{2-[4-chloro-3-(trifluoromethoxy)phenyl]-2-oxoethyl}-4-cyclopropyl-1H-pyrazole-3,5-dicarboxylate (Intermediate 424A, 2.6 g, 5.4 mmol, 95% purity) in acetic acid (45 ml) was added ammonium acetate (4.1 g, 53.6 mmol). The resulting mixture was heated for 4 days at 110° C. After cooling to RT, the reaction mixture was quenched with water. The solid was collected by filtration, washed with water and dried in vacuum to give 1.9 g (77% of theory, 93% purity) of the title compound.

LC/MS [Method 3]: Rₜ=2.24 min; MS (ESIpos): m/z=440 [M−H]⁻.

### Intermediate 426A

### 6-[4-Chloro-3-(trifluoromethoxy)phenyl]-3-cyclopropyl-4-oxo-4,5-dihydropyrazolo[1,5-a]pyrazine-2-carboxylic acid

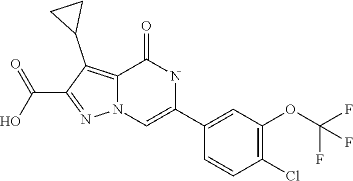 [SMILES: O=C(O)c1nn2cc(-c3ccc(Cl)c(OC(F)(F)F)c3)[nH]c(=O)c2c1C1CC1]

To a mixture of ethyl 6-[4-chloro-3-(trifluoromethoxy)phenyl]-3-cyclopropyl-4-oxo-4,5-dihydropyrazolo[1,5-a]pyrazine-2-carboxylate (Intermediate 425A, 1.9 g, 4.1 mmol, 93% purity) in ethanol (30 ml) was added a solution 1M of lithium hydroxide (20.6 ml). After stirring overnight at RT, the reaction mixture was concentrated under reduced pressure. Then the rest of mixture was diluted with water and acidified with 1N hydrochloric acid. The product was collected by filtration, washed with water and dried in high vacuum to give 1.86 g (quant., 100% purity) of the title compound.

LC/MS [Method 3]: Rₜ=1.75 min; MS (ESIpos): m/z=412 [M−H]⁻.

¹H-NMR (400 MHz, DMSO-d₆): δ [ppm]=13.16 (bs, 1H), 11.62 (bs, 1H), 8.20 (s, 1H), 7.98 (s, 1H), 7.86-7.81 (m, 2H), 2.77-2.67 (m, 1H), 1.27-1.23 (m, 2H), 0.95-0.91 (m, 2H).

### Intermediate 427A

### Diethyl 1-[2-(4-chloro-2-fluoro-5-methylphenyl)-2-oxoethyl]-4-cyclopropyl-1H-pyrazole-3,5-dicarboxylate

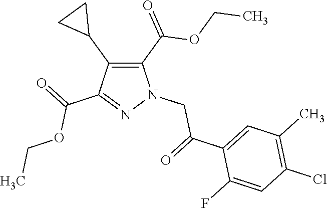 [SMILES: CCOC(=O)c1nn(CC(=O)c2cc(C)c(Cl)cc2F)c(C(=O)OCC)c1C1CC1]

To a solution of diethyl 4-cyclopropyl-1H-pyrazole-3,5-dicarboxylate (Intermediate 187A, 1.0 g, 3.96 mmol) and 2-bromo-1-(4-chloro-2-fluoro-5-methylphenyl)ethanone (1.3 g, 4.8 mmol) in acetone (35 ml) was added potassium carbonate (1.4 g, 9.9 mmol). The resulting mixture was stirred at RT for 1 h. After filtering off the solids, the filter cake was washed with acetone. The filtrate was evaporated under reduced pressure to give 1.5 g (79% of theory, 91% purity) of the title compound.

LC/MS [Method 3]: Rₜ=2.45 min; MS (ESIpos): m/z=437 [M+H]⁺.

### Intermediate 428A

### Ethyl 6-(4-chloro-2-fluoro-5-methylphenyl)-3-cyclopropyl-4-oxo-4,5-dihydropyrazolo[1,5-a]-pyrazine-2-carboxylate

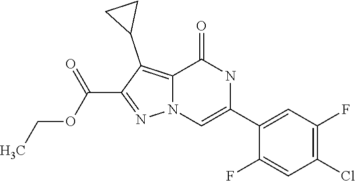 [SMILES: CCOC(=O)c1nn2cc(-c3cc(F)c(Cl)cc3F)[nH]c(=O)c2c1C1CC1]

To a solution of diethyl 1-[2-(4-chloro-2-fluoro-5-methylphenyl)-2-oxoethyl]-4-cyclopropyl-1H-pyrazole-3,5-dicarboxylate (Intermediate 427A, 1.5 g, 3.1 mmol, 91% purity) in acetic acid (18.2 ml) was added ammonium acetate (2.4 g, 31.4 mmol). The resulting mixture was heated for 48 h at 110° C. After cooling to RT, the reaction mixture was poured into ice-water. The solid was collected by filtration, washed with MTBE and dried in vacuum to give 1.5 g (91% of theory, 76% purity) of the title compound.

LC/MS [Method 3]: Rₜ=2.15 min; MS (ESIpos): m/z=390 [M+H]⁺.

### Intermediate 429A

### 6-(4-Chloro-2-fluoro-5-methylphenyl)-3-cyclopropyl-4-oxo-4,5-dihydropyrazolo[1 ,5-a]pyrazine-2-carboxylic acid

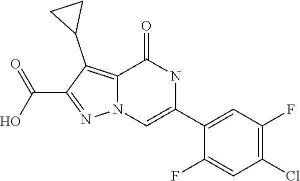 [SMILES: O=C(O)c1nn2cc(-c3cc(F)c(Cl)cc3F)[nH]c(=O)c2c1C1CC1]

To a mixture of ethyl 6-(4-chloro-2-fluoro-5-methylphenyl)-3-cyclopropyl-4-oxo-4,5-dihydropyrazolo[1,5-a]pyrazine-2-carboxylate (Intermediate 428A, 1.5 g, 2.8 mmol, 76% purity) in a mixture THF/methanol (21.5 ml, 5/1) was added a solution 1M of lithium hydroxide (14.2 ml). After stirring overnight at RT, the reaction mixture was concentrated under reduced pressure. Then the rest of mixture was diluted with water and acidified with 1N hydrochloric acid. The product was collected by filtration, washed with water and dried in high vacuum to give 1.2 g (>100% of theory, 100% purity) of the title compound.

LC/MS [Method 3]: Rₜ=1.65 min; MS (ESIpos): m/z=362 [M+H]⁺.

### Intermediate 430A

### Diethyl 4-cyclopropyl-1-[2-(2,3-difluoro-4-methylphenyl)-2-oxoethyl]-1H-pyrazole-3,5-dicarboxylate

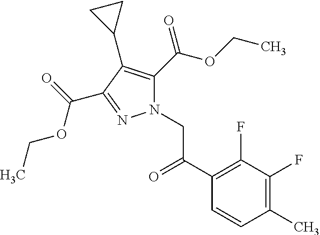 [SMILES: CCOC(=O)c1nn(CC(=O)c2ccc(C)c(F)c2F)c(C(=O)OCC)c1C1CC1]

To a solution of diethyl 4-cyclopropyl-1H-pyrazole-3,5-dicarboxylate (Intermediate 187A, 1.0 g, 3.96 mmol) and 2-bromo-1-(2,3-difluoro-4-methylphenyl)ethanone (2.9 g, 4.7 mmol, 50% purity) in acetone (35 ml) was added potassium carbonate (1.4 g, 9.9 mmol). The resulting mixture was stirred at RT for 2 h. After filtering off the solids, the filter cake was washed with acetone. The filtrate was evaporated under reduced pressure to give 3.45 g (87% of theory, 65% purity) of the title compound.

LC/MS [Method 3]: Rₜ=2.29 min; MS (ESIpos): m/z=421 [M+H]⁺.

### Intermediate 431A

### Ethyl 3-cyclopropyl-6-(2,3-difluoro-4-methylphenyl)-4-oxo-4,5-dihydropyrazolo[1,5-a]pyrazine-2-carboxylate

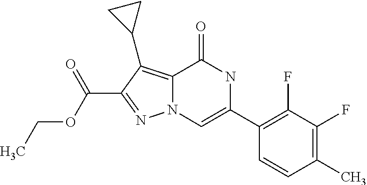 [SMILES: CCOC(=O)c1nn2cc(-c3ccc(C)c(F)c3F)[nH]c(=O)c2c1C1CC1]

To a solution of diethyl 4-cyclopropyl-1-[2-(2,3-difluoro-4-methylphenyl)-2-oxoethyl]-1H-pyrazole-3,5-dicarboxylate (Intermediate 430A, 3.45 g, 5.3 mmol, 65% purity) in acetic acid (45 ml) was added ammonium acetate (4.1 g, 53.3 mmol). The resulting mixture was heated overnight at 110° C. After cooling to RT, the reaction mixture was quenched with water. The solid was collected by filtration, washed with water and dried in vacuum to give 1.6 g (51% of theory, 62% purity) of the title compound.

LC/MS [Method 3]: Rₜ=1.99 min; MS (ESIpos): m/z=372 [M−H]⁻.

### Intermediate 432A

### 3-Cyclopropyl-6-(2,3-difluoro-4-methylphenyl)-4-oxo-4,5-dihydropyrazolo[1,5-a]pyrazine-2-carboxylic acid

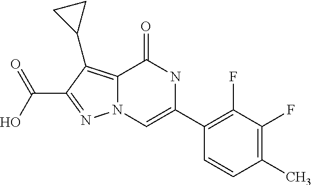 [SMILES: Cc1ccc(-c2cn3nc(C(=O)O)c(C4CC4)c3c(=O)[nH]2)c(F)c1F]

To a mixture of ethyl 3-cyclopropyl-6-(2,3-difluoro-4-methylphenyl)-4-oxo-4,5-dihydropyrazolo-[1,5-a]pyrazine-2-carboxylate (Intermediate 431A, 1.6 g, 4.4 mmol, 62% purity) in a mixture THF/methanol (35.3 ml, 5/1) was added a solution 1M of lithium hydroxide (22.1 ml). After stirring at RT for 48 h, the reaction mixture was concentrated under reduced pressure. Then the rest of mixture was diluted with water and acidified with formic acid. The product was collected by filtration, washed with water and dried in high vacuum to give 1.2 g (63% of theory, 95% purity) of the title compound.

LC/MS [Method 3]: Rₜ=1.50 min; MS (ESIpos): m/z=346 [M+H]⁺.

### Intermediate 433A

### 2-Bromo-1-(5,6-dimethylpyridin-2-yl)ethanone

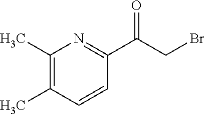

A solution of 1-(5,6-dimethylpyridin-2-yl)ethanone (775 mg, 5.2 mmol) and phenyltrimethylammonium tribromide (1.9 g, 5.2 mmol) in THF (15.5 ml) was stirred at 50° C. overnight. The ammonium salts were filtered off, and the filter cake was washed with acetonitrile. The filtrate was evaporated under reduced pressure to give 248.7 mg (31% of theory, 49% purity) of the title compound which was used in the next step without further purification.

LC/MS [Method 3]: Rₜ=1.84 min; MS (ESIpos): m/z=228 [M+H]⁺.

### Intermediate 434A

### Diethyl 4-cyclopropyl-1-[2-(5,6-dimethylpyridin-2-yl)-2-oxoethyl]-1H-pyrazole-3,5-dicarboxylate

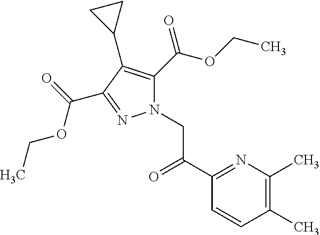 [SMILES: CCOC(=O)c1nn(CC(=O)c2ccc(C)c(C)n2)c(C(=O)OCC)c1C1CC1]

To a solution of diethyl 4-cyclopropyl-1H-pyrazole-3,5-dicarboxylate (Intermediate 187A, 184.6 mg, 0.7 mmol) and 2-bromo-1-(5,6-dimethylpyridin-2-yl)ethanone (Intermediate 433A, 248.7 mg, 0.8 mmol, 49% purity) in acetone (6.4 ml) was added potassium carbonate (353.9 mg, 2.6 mmol). The resulting mixture was stirred at RT overnight. After filtering off the solids, the filter cake was washed with acetone. The filtrate was evaporated under reduced pressure and purified by preparative HPLC (Method P16) to give 180.0 mg (57% of theory, 92% purity) of the title compound.

LC/MS [Method 3]: Rₜ=2.27 min; MS (ESIpos): m/z=400 [M+H]⁺.

### Intermediate 435A

### Ethyl 3-cyclopropyl-6-(5,6-dimethylpyridin-2-yl)-4-oxo-4,5-dihydropyrazolo[1,5-a]pyrazine-2-carboxylate

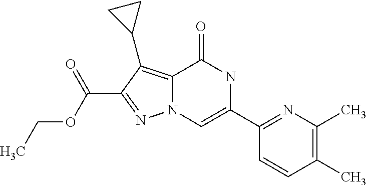 [SMILES: CCOC(=O)c1nn2cc(-c3ccc(C)c(C)n3)[nH]c(=O)c2c1C1CC1]

To a solution of diethyl 4-cyclopropyl-1-[2-(5,6-dimethylpyridin-2-yl)-2-oxoethyl]-1H-pyrazole-3,5-dicarboxylate (Intermediate 434A, 180.0 mg, 0.4 mmol, 92% purity) in acetic acid (2.0 ml) was added ammonium acetate (319.5 mg, 4.1 mmol). The resulting mixture was heated for 2 days at 110° C. After cooling to RT, the reaction mixture was poured into ice-water. The solid was collected by filtration and washed with water. The pH was adjusted to 5-6 with a solution 1N of sodium hydroxide, and dried in vacuum to give 132.0 mg (90% of theory, 92% purity) of the title compound.

LC/MS [Method 3]: Rₜ=2.14 min; MS (ESIpos): m/z=353 [M+H]⁺.

### Intermediate 436A

### 3-Cyclopropyl-6-(5,6-dimethylpyridin-2-yl)-4-oxo-4,5-dihydropyrazolo[1,5-a]pyrazine-2-carboxylic acid

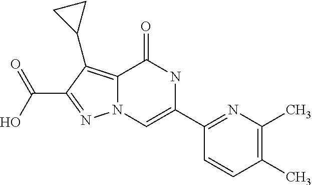 [SMILES: Cc1ccc(-c2cn3nc(C(=O)O)c(C4CC4)c3c(=O)[nH]2)nc1C]

To a mixture of ethyl 3-cyclopropyl-6-(5,6-dimethylpyridin-2-yl)-4-oxo-4,5-dihydropyrazolo[1,5-a]-pyrazine-2-carboxylate (Intermediate 435A, 132 mg, 0.38 mmol, 92% purity) in a mixture THF/methanol (3 ml, 5/1) was added a solution 1M of lithium hydroxide (1.87 ml). After stirring overnight at RT, the reaction mixture was concentrated under reduced pressure. Then the rest of mixture was diluted with water and acidified with formic acid. The product was collected by filtration, washed with water and dried in high vacuum to give 121.0 mg (100% of theory, 100% purity) of the title compound.

LC/MS [Method 3]: Rₜ=1.56 min; MS (ESIpos): m/z=325 [M+H]⁺.

### Intermediate 437A

### 2-Bromo-1-[3-chloro-4-(trifluoromethyl)phenyl]ethanone

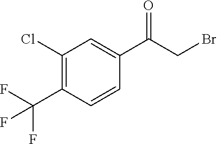 [SMILES: O=C(CBr)c1ccc(C(F)(F)F)c(Cl)c1]

A solution of 1-[3-chloro-4-(trifluoromethyl)phenyl]ethanone (2.0 g, 8.9 mmol) and phenyltrimethylammonium tribromide (4.2 g, 10.8 mmol) in THF (34.2 ml) was stirred at 50° C. for 1 h. The ammonium salts were filtered off, and the filter cake was washed with THF. The filtrate was evaporated under reduced pressure to give 3.8 g (98% of theory, 50% purity) of the title compound which was used in the next step without further purification.

### Intermediate 438A

### Diethyl 1-{2-[3-chloro-4-(trifluoromethyl)phenyl]-2-oxoethyl}-4-cyclopropyl-1H-pyrazole-3,5-dicarboxylate

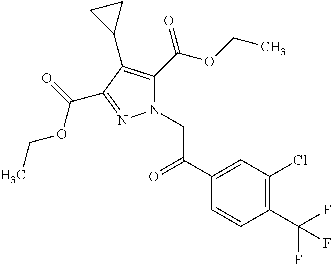 [SMILES: CCOC(=O)c1nn(CC(=O)c2ccc(C(F)(F)F)c(Cl)c2)c(C(=O)OCC)c1C1CC1]

To a solution of diethyl 4-cyclopropyl-1H-pyrazole-3,5-dicarboxylate (Intermediate 187A, 1.0 g, 3.9 mmol) and 2-bromo-1-[3-chloro-4-(trifluoromethyl)phenyl]ethanone (Intermediate 437A, 2.8 g, 4.8 mmol, 50% purity) in acetone (35 ml) was added potassium carbonate (1.37 g, 9.9 mmol). The resulting mixture was stirred at RT for 1 h. After filtering off the solids, the filter cake was washed with acetone. The filtrate was evaporated under reduced pressure to give 2.8 g (>100% of theory, 70% purity) of the title compound.

LC/MS [Method 3]: Rₜ=2.44 min; MS (ESIpos): m/z=473 [M+H]⁺.

### Intermediate 439A

### Ethyl 6-[3-chloro-4-ftrifluoromethyflphenyl]-3-cyclopropyl-4-oxo-4,5-dihydropyrazolo[1,5-a]pyrazine-2-carboxylate

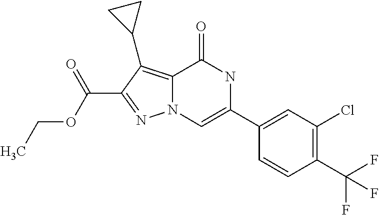 [SMILES: CCOC(=O)c1nn2cc(-c3ccc(C(F)(F)F)c(Cl)c3)[nH]c(=O)c2c1C1CC1]

To a solution of diethyl 1-{2-[3-chloro-4-(trifluoromethyl)phenyl]-2-oxoethyl}-4-cyclopropyl-1H-pyrazole-3,5-dicarboxylate (Intermediate 438A, 3.2 g, 4.7 mmol, 70% purity) in acetic acid (40 ml) was added ammonium acetate (3.6 g, 47.5 mmol). The resulting mixture was heated for 48 h at 110° C. After cooling to RT, the reaction mixture was quenched with water. The solid was collected by filtration, washed with water and dried in vacuum to give 1.99 g (98% of theory, 100% purity) of the title compound.

LC/MS [Method 3]: Rₜ=2.20 min; MS (ESIpos): m/z=424 [M−H]⁻.

### Intermediate 440A

### 6-[3-Chloro-4-(trifluoromethyl)phenyl]-3-cyclopropyl-4-oxo-4,5-dihydropyrazolo[1,5-a]pyrazine-2-carboxylic acid

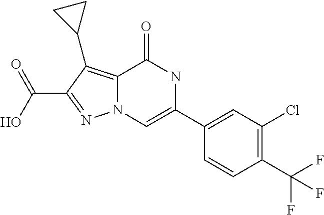 [SMILES: O=C(O)c1nn2cc(-c3ccc(C(F)(F)F)c(Cl)c3)[nH]c(=O)c2c1C1CC1]

To a mixture of ethyl 6-[3-chloro-4-(trifluoromethyl)phenyl]-3-cyclopropyl-4-oxo-4,5-dihydropyrazolo[1,5-a]pyrazine-2-carboxylate (Intermediate 439A, 1.99 g, 4.7 mmol) in a mixture THF/methanol (35 ml, 5/1) was added a solution 1M of lithium hydroxide (23.4 ml). After stirring for 6 h at RT, the reaction mixture was concentrated under reduced pressure. Then the rest of mixture was diluted with water and acidified with 1N hydrochloric acid. The product was collected by filtration, washed with water and dried in high vacuum to give 1.47 g (71% of theory, 90% purity) of the title compound.

LC/MS [Method 3]: Rₜ=1.70 min; MS (ESIpos): m/z=396 [M−H]⁻.

### Intermediate 441A

### Diethyl 4-cyclopropyl-1-[2-(1-methyl-1H-benzimidazol-5-yl)-2-oxoethyl]-1H-pyrazole-3,5-dicarboxylate

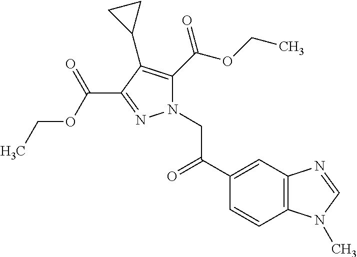 [SMILES: CCOC(=O)c1nn(CC(=O)c2ccc3c(c2)ncn3C)c(C(=O)OCC)c1C1CC1]

To a solution of diethyl 4-cyclopropyl-1H-pyrazole-3,5-dicarboxylate (Intermediate 187A, 327.8 mg, 1.2 mmol, 96% purity) and 2-bromo-1-(1-methyl-1H-benzimidazol-5-yl)ethanone (500.0 mg, 1.5 mmol) in acetone (11 ml) was added potassium carbonate (431.0 mg, 1.5 mmol). The resulting mixture was stirred at RT for 24 h. After filtering off the solids, the filter cake was washed with acetone. The filtrate was evaporated under reduced pressure to give 508.0 mg (82% of theory, 85% purity) of the title compound.

LC/MS [Method 11]: Rₜ=0.93 min; MS (ESIpos): m/z=425 [M+H]⁺.

### Intermediate 442A

### Ethyl 3-cyclopropyl-6-(1-methyl-1H-benzimidazol-5-yl)-4-oxo-4,5-dihydropyrazolo[1,5-a]pyrazine-2-carboxylate

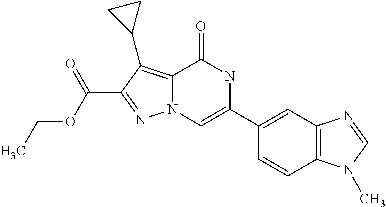 [SMILES: CCOC(=O)c1nn2cc(-c3ccc4c(c3)ncn4C)[nH]c(=O)c2c1C1CC1]

To a solution of diethyl 4-cyclopropyl-1-[2-(1-methyl-1H-benzimidazol-5-yl)-2-oxoethyl]-1H-pyrazole-3,5-dicarboxylate (Intermediate 441A, 508.0 mg, 1.0 mmol, 85% purity) in acetic acid (4.4 ml) was added ammonium acetate (784.1 mg, 10.1 mmol). The resulting mixture was heated for 48 h at 110° C. After cooling to RT, the reaction mixture was quenched with water. The solid was collected by filtration, washed with water and dried in vacuum to give 227.0 mg (64% of theory, 90% purity) of the title compound.

LC/MS [Method 3]: Rₜ=1.32 min; MS (ESIpos): m/z=378 [M+H]⁺.

### Intermediate 443A

### 3-Cyclopropyl-6-(1-methyl-1H-benzimidazol-5-yl)-4-oxo-4,5-dihydropyrazolo[1 ,5-a]pyrazine-2-carboxylic acid

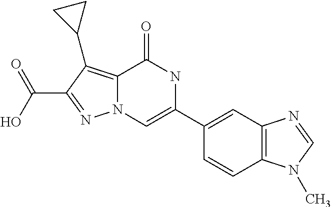 [SMILES: Cn1cnc2cc(-c3cn4nc(C(=O)O)c(C5CC5)c4c(=O)[nH]3)ccc21]

To a mixture of ethyl 3-cyclopropyl-6-(1-methyl-1H-benzimidazol-5-yl)-4-oxo-4,5-dihydropyrazolo[1,5-a]pyrazine-2-carboxylate (Intermediate 442A, 227.0 mg, 0.6 mmol) in a mixture THF/methanol (6 ml, 5/1) was added a solution 1M of lithium hydroxide (3.0 ml). After stirring for 24 h at RT, the reaction mixture was concentrated under reduced pressure. Then the rest of mixture was diluted with water and acidified with 3N hydrochloric acid. The product was collected by filtration, washed with water and dried in high vacuum to give 215.0 mg (>100% of theory, 100% purity) of the title compound.

LC/MS [Method 3]: Rₜ=0.80 min; MS (ESIpos): m/z=350 [M+H]⁺.

### Intermediate 444A

### Diethyl 4-cyclopropyl-1-{2-[2,3-difluoro-4-(trifluoromethyl)phenyl]-2-oxoethyl}-1H-pyrazole-3,5-dicarboxylate

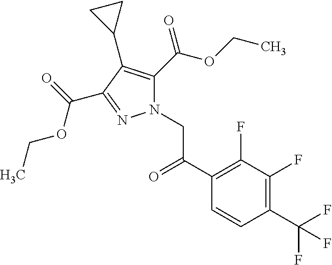 [SMILES: CCOC(=O)c1nn(CC(=O)c2ccc(C(F)(F)F)c(F)c2F)c(C(=O)OCC)c1C1CC1]

To a solution of diethyl 4-cyclopropyl-1H-pyrazole-3,5-dicarboxylate (Intermediate 187A, 1.0 g, 3.9 mmol) and 2-bromo-1-[3-chloro-4-(trifluoromethyl)phenyl]ethanone (2.9 g, 4.8 mmol, 50% purity) in acetone (35 ml) was added potassium carbonate (1.37 g, 9.9 mmol). The resulting mixture was stirred at RT for 2 h. After filtering off the solids, the filter cake was washed with acetone. The filtrate was evaporated under reduced pressure to give 1.7 g (88% of theory, 100% purity) of the title compound.

LC/MS [Method 3]: Rₜ=2.40 min; MS (ESIpos): m/z=475 [M+H]⁺.

### Intermediate 445A

### Ethyl 3-cyclopropyl-6-[2,3-difluoro-4-(trifluoromethyl)phenyl]-4-oxo-4,5-dihydropyrazolo[1,5-a]pyrazine-2-carboxylate

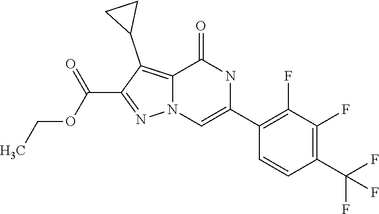

To a solution of diethyl 4-cyclopropyl-1-{2-[2,3-difluoro-4-(trifluoromethyl)phenyl]-2-oxoethyl}-1H-pyrazole-3,5-dicarboxylate (Intermediate 444A, 1.7 g, 3.5 mmol) in acetic acid (20.6 ml) was added ammonium acetate (2.7 g, 34.9 mmol). The resulting mixture was heated for 3 days at 110° C. After cooling to RT, the reaction mixture was quenched with water. The solid was collected by filtration, washed with water and dried in vacuum to give 1.30 g (85% of theory, 98% purity) of the title compound.

LC/MS [Method 3]: Rₜ=2.10 min; MS (ESIpos): m/z=426 [M−H]⁻.

### Intermediate 446A

### 3-Cyclopropyl-6-[2,3-difluoro-4-(trifluoromethyl)phenyl]-4-oxo-4,5-dihydropyrazolo[1,5-a]-pyrazine-2-carboxylic acid

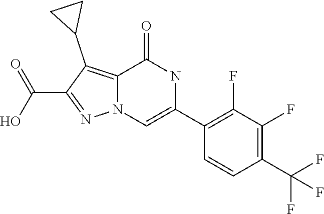

To a mixture of ethyl 3-cyclopropyl-6-[2,3-difluoro-4-(trifluoromethyl)phenyl]-4-oxo-4,5-dihydropyrazolo[1,5-a]pyrazine-2-carboxylate (Intermediate 445A, 1.30 g, 2.9 mmol) in a mixture THF/methanol (22.6 ml, 5/1) was added a solution 1M of lithium hydroxide (14.9 ml). After stirring at RT overnight, the reaction mixture was concentrated under reduced pressure. Then the rest of mixture was diluted with water and acidified with 1N hydrochloric acid. The product was collected by filtration, washed with water and dried in high vacuum to give 1.38 g (>100% of theory, 100% purity) of the title compound.

LC/MS [Method 3]: Rₜ=1.64 min; MS (ESIpos): m/z=398 [M−H]⁻.

### Intermediate 447A

### 3-Ethyl 5-methyl 1-[2-(4-chloro-3-methylphenyl)-2-oxoethyl]-4-(methoxymethyl)-1H-pyrazole-3,5-dicarboxylate and 5-ethyl 3-methyl 1-[2-(4-chloro-3-methylphenyl)-2-oxoethyl]-4-(methoxymethyl)-1H-pyrazole-3,5-dicarboxylate (mixture of unknown ratio)

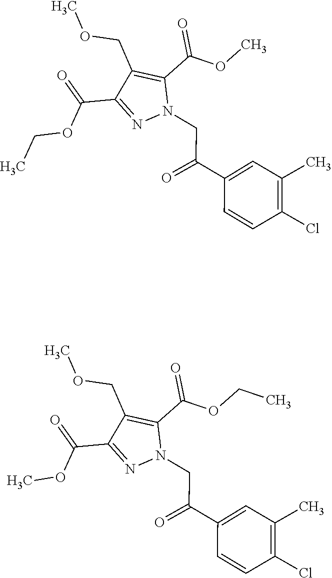 [SMILES: CCOC(=O)c1nn(CC(=O)c2ccc(Cl)c(C)c2)c(C(=O)OC)c1COC ; CCOC(=O)c1c(COC)c(C(=O)OC)nn1CC(=O)c1ccc(Cl)c(C)c1]

To a solution of 5-ethyl 3-methyl 4-(methoxymethyl)-1H-pyrazole-3,5-dicarboxylate (Intermediate 38A, 1.6 g, 4.1 mmol, 62% purity) and 2-bromo-1-(4-chloro-3-methylphenyl)ethanone (1.2 g, 4.9 mmol) in acetone (36 ml) was added potassium carbonate (1.4 g, 4.9 mmol). The resulting mixture was stirred at RT for 2 h. After filtering off the solids, the filter cake was washed with acetone. The filtrate was evaporated under reduced pressure to give 2.58 g (89% of theory, 58% purity, mixture of unknown ratio) of the title compounds.

LC/MS [Method 3]: Rₜ=2.08 min; MS (ESIpos): m/z=409 [M+H]⁺.

### Intermediate 448A

### Ethyl 6-(4-chloro-3-methylphenyl)-3-(methoxymethyl)-4-oxo-4,5-dihydropyrazolo[1,5-a]pyrazine-2-carboxylate and methyl 6-(4-chloro-3-methylphenyl)-3-(methoxymethyl)-4-oxo-4,5-dihydropyrazolo[1,5-a]pyrazine-2-carboxylate (mixture 1:1)

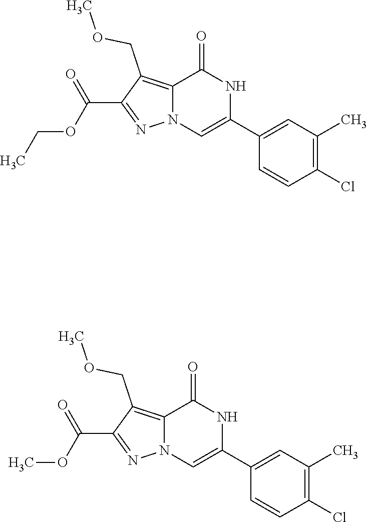

To a solution of the 3-ethyl 5-methyl 1-[2-(4-chloro-3-methylphenyl)-2-oxoethyl]-4-(methoxymethyl)-1H-pyrazole-3,5-dicarboxylate and 5-ethyl 3-methyl 1-[2-(4-chloro-3-methylphenyl)-2-oxoethyl]-4-(methoxymethyl)-1H-pyrazole-3,5-dicarboxylate (Intermediate Intermediate 447A, 2.58 g, 3.7 mmol, 58% purity, mixture of unknown ratio) in acetic acid (30.9 ml) was added ammonium acetate (2.84 g, 36.8 mmol). The resulting mixture was heated overnight at 110° C. After cooling to RT, the reaction mixture was quenched with water. The solid was collected by filtration, washed with water and dried in vacuum to give 1.2 g (54% of theory, 61% purity, mixture 1:1) of the title compounds.

LC/MS [Method 3]: Rₜ=1.73 min; MS (ESIpos): m/z=360 [M−H]⁻1.88 min; MS (ESIpos): m/z=374 [M−H]⁻.

### Intermediate 449A

### 6-(4-Chloro-3-methylphenyl)-3-(methoxymethyl)-4-oxo-4,5-dihydropyrazolo[1 ,5-a]pyrazine-2-carboxylic acid

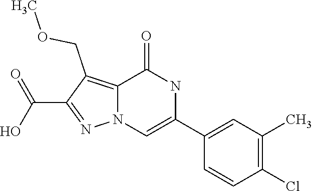 [SMILES: COCc1c(C(=O)O)nn2cc(-c3ccc(Cl)c(C)c3)[nH]c(=O)c12]

To a solution of ethyl 6-(4-chloro-3-methylphenyl)-3-(methoxymethyl)-4-oxo-4,5-dihydropyrazolo-[1,5-a]pyrazine-2-carboxylate and methyl 6-(4-chloro-3-methylphenyl)-3-(methoxymethyl)-4-oxo-4,5-dihydropyrazolo[1,5-a]pyrazine-2-carboxylate (mixture 1:1) (Intermediate 448A, 1.2 g, 3.2 mmol) in a mixture THF/methanol (24.1 ml, 5/1) was added a 1.0 M solution of lithium hydroxide (15.9 ml). After stirring at RT overnight, the reaction mixture was concentrated under reduced pressure. Then the rest of mixture was diluted with water and acidified with 3N hydrochloric acid. The product was collected by filtration, washed with water and dried in high vacuum to give 888.6 mg (62% of theory, 77% purity) of the title compound.

LC/MS [Method 11]: Rₜ=0.77 min; MS (ESIpos): m/z=346 [M−H]⁻.

### Intermediate 450A

### 3-Ethyl 5-methyl 1-[2-(4-chloro-3-fluorophenyl)-2-oxoethyl]-4-(methoxymethyl)-1H-pyrazole-3,5-dicarboxylate and 5-ethyl 3-methyl 1-[2-(4-chloro-3-fluorophenyl)-2-oxoethyl]-4-(methoxymethyl)-1H-pyrazole-3,5-dicarboxylate (mixture of unknown ratio)

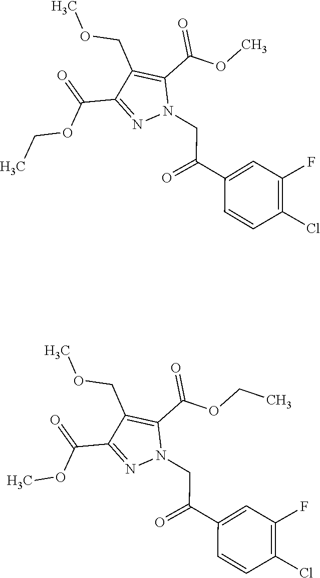 [SMILES: CCOC(=O)c1nn(CC(=O)c2ccc(Cl)c(F)c2)c(C(=O)OC)c1COC ; CCOC(=O)c1c(COC)c(C(=O)OC)nn1CC(=O)c1ccc(Cl)c(F)c1]

To a solution of 5-ethyl 3-methyl 4-(methoxymethyl)-1H-pyrazole-3,5-dicarboxylate (Intermediate 38A, 900.0 mg, 1.2 mmol, 33% purity) and 2-bromo-1-(4-chloro-3-fluorophenyl)ethanone (Intermediate 255A, 510.3 mg, 1.5 mmol, 74% purity) in acetone (11 ml) was added potassium carbonate (430.0 mg, 3.1 mmol). The resulting mixture was stirred at RT for 2 h. After filtering off the solids, the filter cake was washed with acetone. The filtrate was evaporated under reduced pressure to give 1.1 g (98% of theory, 46% purity, mixture of unknown ratio) of the title compounds.

LC/MS [Method 11]: Rₜ=3.32 min; MS (ESIpos): m/z=411 [M−H]⁻.

### Intermediate 451A

### Ethyl 6-(4-chloro-3-fluorophenyl)-3-(methoxymethyl)-4-oxo-4,5-dihydropyrazolo[1,5-a]pyrazine-2-carboxylate and methyl 6-(4-chloro-3-fluorophenyl)-3-(methoxymethyl)-4-oxo-4,5-dihydropyrazolo[1,5-a]pyrazine-2-carboxylate (mixture 1:1)

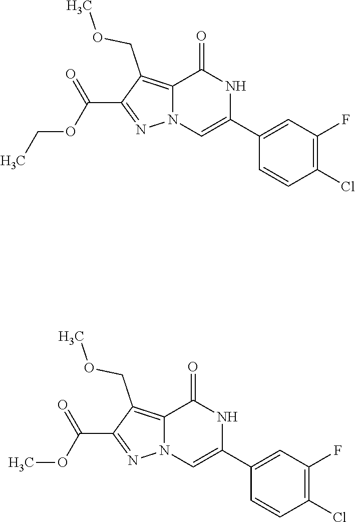 [SMILES: CCOC(=O)c1nn2cc(-c3ccc(Cl)c(F)c3)[nH]c(=O)c2c1COC ; COCc1c(C(=O)OC)nn2cc(-c3ccc(Cl)c(F)c3)[nH]c(=O)c12]

To a solution of the mixture 3-ethyl 5-methyl 1-[2-(4-chloro-3-fluorophenyl)-2-oxoethyl]-4-(methoxymethyl)-1H-pyrazole-3,5-dicarboxylate and 5-ethyl 3-methyl 1-[2-(4-chloro-3-fluorophenyl)-2-oxoethyl]-4-(methoxymethyl)-1H-pyrazole-3,5-dicarboxylate (Intermediate Intermediate 450A, 1.1 g, 1.2 mmol, 46% purity, unknown ratio) in acetic acid (10.3 ml) was added ammonium acetate (938.9 mg, 12.2 mmol). The resulting mixture was heated for two days at 110° C. After cooling to RT, the reaction mixture was quenched with water. The solid was collected by filtration, washed with water and dried in vacuum to give 301.0 mg (27% of theory, 87% purity, mixture 1:1) of the title compounds.

LC/MS [Method 3]: Rₜ=1.61min; MS (ESIpos): m/z=364 [M−H]⁻; 1.75 min; MS (ESIpos): m/z=378 [M−H]⁻.

### Intermediate 452A

### 6-(4-Chloro-3-fluorophenyl)-3-(methoxymethyl)-4-oxo-4,5-dihydropyrazolo[1,5-a]pyrazine-2-carboxylic acid

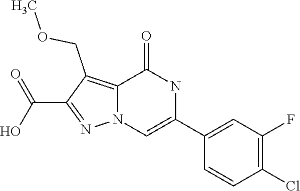 [SMILES: COCc1c(C(=O)O)nn2cc(-c3ccc(Cl)c(F)c3)[nH]c(=O)c12]

To a solution of ethyl 6-(4-chloro-3-fluorophenyl)-3-(methoxymethyl)-4-oxo-4,5-dihydropyrazolo-[1,5-a]pyrazine-2-carboxylate and methyl 6-(4-chloro-3-fluorophenyl)-3-(methoxymethyl)-4-oxo-4,5-dihydropyrazolo[1,5-a]pyrazine-2-carboxylate (Intermediate Intermediate 451A, 301.0 mg, 0.8 mmol, 87% purity, mixture 1:1) in a mixture THF/methanol (6 ml, 5/1) was added a solution 1M of lithium hydroxide (3.9 ml). After stirring at RT overnight, the reaction mixture was concentrated under reduced pressure. Then the rest of mixture was diluted with water and acidified with 1N hydrochloric acid. The product was collected by filtration, washed with water and dried in high vacuum to give 225.9 mg (71% of theory, 88% purity) of the title compound.

LC/MS [Method 3]: Rₜ=1.31 min; MS (ESIpos): m/z=352 [M+H]⁺.

### Intermediate 453A

### 3-Ethyl 5-methyl 1-[2-(3-chloro-4-methylphenyl)-2-oxoethyl]-4-(methoxymethyl)-1H-pyrazole-3,5-dicarboxylate and 5-ethyl 3-methyl 1-[2-(3-chloro-4-methylphenyl)-2-oxoethyl]-4-(methoxymethyl)-1H-pyrazole-3,5-dicarboxylate (mixture of unknown ratio)

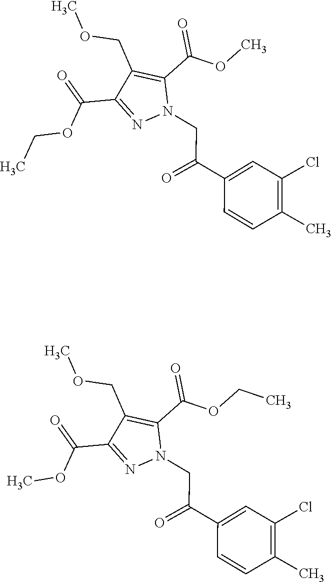

To a solution of 5-ethyl 3-methyl 4-(methoxymethyl)-1H-pyrazole-3,5-dicarboxylate (Intermediate 38A, 592.0 mg, 1.3 mmol, 55% purity) and 2-bromo-1-(3-chloro-4-methylphenyl)ethanone (397.0 mg, 1.6 mmol) in acetone (11.8 ml) was added potassium carbonate (461.9 mg, 3.3 mmol). The resulting mixture was stirred at RT for 2 h. After filtering off the solids, the filter cake was washed with acetone. The filtrate was evaporated under reduced pressure to give 802.8 mg (quant., 49% purity, mixture of unknown ratio) of the title compounds.

LC/MS [Method 11]: Rₜ=2.07 min; MS (ESIpos): m/z=407 [M−H]⁻.

### Intermediate 454A

### Ethyl 6-(3-chloro-4-methylphenyl)-3-(methoxymethyl)-4-oxo-4,5-dihydropyrazolo[1,5-a]pyrazine-2-carboxylate and methyl 6-(3-chloro-4-methylphenyl)-3-(methoxymethyl)-4-oxo-4,5-dihydropyrazolo[1,5-a]pyrazine-2-carboxylate (mixture 1:1)

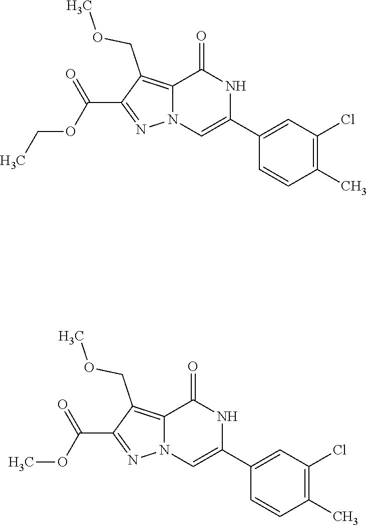 [SMILES: CCOC(=O)c1nn2cc(-c3ccc(C)c(Cl)c3)[nH]c(=O)c2c1COC ; COCc1c(C(=O)OC)nn2cc(-c3ccc(C)c(Cl)c3)[nH]c(=O)c12]

To a solution of diethyl 1-[2-(3-chloro-4-methylphenyl)-2-oxoethyl]-4-(methoxymethyl)-1H-pyrazole-3,5-dicarboxylate (Intermediate 453A, 802.8 mg, 0.9 mmol, 49% purity, mixture of unknown ratio) in acetic acid (8.1 ml) was added ammonium acetate (741.6 mg, 9.6 mmol). The resulting mixture was heated overnight at 110° C. After cooling to RT, the reaction mixture was quenched with water. The solid was collected by filtration, washed with water and dried in vacuum to give 260.0 mg (24% of theory, 68% purity, mixture 1:1) of the title compounds.

LC/MS [Method 3]: Rₜ=1.72 min; MS (ESIpos): m/z=360 [M−H]⁻; 1.86 min; MS (ESIpos): m/z=374 [M−H]⁻.

### Intermediate 455A

### 6-(3-Chloro-4-methylphenyl)-3-(methoxymethyl)-4-oxo-4,5-dihydropyrazolo[1 ,5-a]pyrazine-2-carboxylic acid

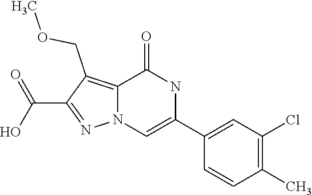 [SMILES: COCc1c(C(=O)O)nn2cc(-c3ccc(C)c(Cl)c3)[nH]c(=O)c12]

To a solution of ethyl 6-(3-chloro-4-methylphenyl)-3-(methoxymethyl)-4-oxo-4,5-dihydropyrazolo-[1,5-a]pyrazine-2-carboxylate and methyl 6-(3-chloro-4-methylphenyl)-3-(methoxymethyl)-4-oxo-4,5-dihydropyrazolo[1,5-a]pyrazine-2-carboxylate (Intermediate 454A, 260.0 mg, 0.7 mmol, 68% purity, mixture 1:1) in a mixture THF/methanol (15.8 ml, 5/1) was added a 1.0 M solution of lithium hydroxide (3.4 ml). After stirring at RT for 6 h, the reaction mixture was concentrated under reduced pressure. Then the rest of mixture was diluted with water and acidified with 1N hydrochloric acid. The product was collected by filtration and washed with water. The resulting product was stirred in MTBE, filtered and dried in high vacuum to give 174.0 mg (55% of theory, 76% purity) of the title compound.

LC/MS [Method 3]: Rₜ=1.40 min; MS (ESIpos): m/z=346 [M−H]⁻.

### Intermediate 456A

### Dimethyl 1-[2-(2,3-dihydro-1,4-benzodioxin-6-yl)-2-oxoethyl]-4-(methoxymethyl)-1H-pyrazole-3,5-dicarboxylate

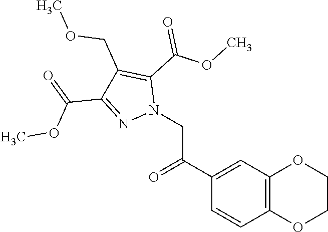 [SMILES: COCc1c(C(=O)OC)nn(CC(=O)c2ccc3c(c2)OCCO3)c1C(=O)OC]

To a solution of dimethyl 4-(methoxymethyl)-1H-pyrazole-3,5-dicarboxylate (Intermediate 39A, 2.0 g, 8.8 mmol) and 2-bromo-1-(2,3-dihydro-1,4-benzodioxin-6-yl)ethanone (Intermediate 99A, 2.9 g, 9.6 mmol, 85% purity) in acetone (50.0 ml) was added potassium carbonate (2.4 g, 17.5 mmol). The resulting mixture was stirred at RT overnight. After filtering off the solids, the filter cake was washed with acetone. The filtrate was evaporated under reduced pressure and purified by flash chromatography on silica gel (eluent: petroleum ether-ethyl acetate 100%, 3:1) to give 3.7 g (68% of theory, 65% purity) of the title compound.

LC/MS [Method 18]: Rₜ=0.90 min; MS (ESIpos): m/z=405 [M+H]⁺.

¹H-NMR (300 MHz, DMSO-d₆): δ [ppm]=7.44-7.60 (m, 2H), 7.03-7.08 (m, 1H), 6.12 (s, 2H), 4.76 (s, 2H), 4.28-4.39 (m, 4H), 3.87 (s, 3H), 3.76 (s, 3H), 3.26 (s, 3H).

### Intermediate 457A

### Methyl 6-(2,3-dihydro-1,4-benzodioxin-6-yl)-3-(methoxymethyl)-4-oxo-4,5-dihydropyrazolo[1 ,5-a]-pyrazine-2-carboxylate

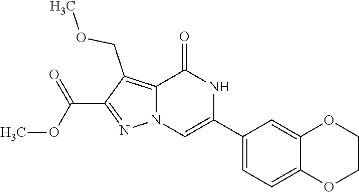 [SMILES: COCc1c(C(=O)OC)nn2cc(-c3ccc4c(c3)OCCO4)[nH]c(=O)c12]

To a solution of dimethyl 1-[2-(2,3-dihydro-1,4-benzodioxin-6-yl)-2-oxoethyl]-4-(methoxymethyl)-1H-pyrazole-3,5-dicarboxylate (Intermediate 456A, 3.7 g, 5.9 mmol, 65% purity) in acetic acid (50.0 ml) was added ammonium acetate (9.1 g, 118.9 mmol). The resulting mixture was heated overnight at 110° C. After cooling to RT, the reaction mixture was quenched with water. The solid was collected by filtration, washed with water and dried in vacuum to give 2.5 g (61% of theory, 54% purity) of the title compound.

LC/MS [Method 18]: Rₜ=0.90 min; MS (ESIpos): m/z=372 [M−H]⁻.

¹H-NMR (400 MHz, DMSO-d₆): δ [ppm]=11.66 (s, 1H), 8.06 (s, 1H), 7.23-7.32 (m, 2H), 6.97 (d, 1H), 4.91 (s, 2H), 4.28-4.32 (m, 4H), 3.85 (s, 3H), 3.26 (s, 3H).

### Intermediate 458A

### 6-(2,3-Dihydro-1,4-benzodioxin-6-yl)-3-(methoxymethyl)-4-oxo-4,5-dihydropyrazolo[1,5-a]-pyrazine-2-carboxylic acid

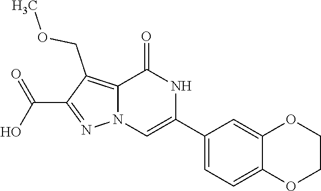 [SMILES: COCc1c(C(=O)O)nn2cc(-c3ccc4c(c3)OCCO4)[nH]c(=O)c12]

To a mixture of methyl 6-(2,3-dihydro-1,4-benzodioxin-6-yl)-3-(methoxymethyl)-4-oxo-4,5-dihydropyrazolo[1,5-a]pyrazine-2-carboxylate (Intermediate 457A, 2.5 g, 3.6 mmol, 54% purity) in a mixture ethanol/water (15.8 ml, 9/1) was added sodium hydroxide (1.4 g, 36.3 mmol). After stirring at RT for 4 h, the reaction mixture was diluted with water (80 ml) and extracted with ethyl acetate (2×40 ml). Then the aqueous layer was acidified with 1N hydrochloric acid. The product was collected by filtration, washed with water and dried in high vacuum to give 829.6 mg (57% of theory, 89% purity) of the title compound.

LC/MS [Method 18]: Rₜ=0.94 min; MS (ESIpos): m/z=358 [M+H]⁺.

¹H-NMR (400 MHz, DMSO-d₆): δ [ppm]=13.15 (bs, 1H), 11.58 (s, 1H), 8.01 (s, 1H), 7.23-7.31 (m, 2H), 6.96 (d, 1H), 4.91 (s, 2H), 4.28-4.31 (m, 4H), 3.25 (s, 3H).

### Intermediate 459A

### Ethyl 4,4-difluoro-3-oxo-2-(triphenylphosphoranylidene)butanoate

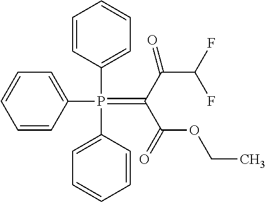 [SMILES: CCOC(=O)C(C(=O)C(F)F)=P(c1ccccc1)(c1ccccc1)c1ccccc1]

To a solution of ethyl (triphenylphosphoranylidene)acetate (81.9 g, 235.0 mmol) and triethylamine (39.3 mL, 282.0 mmol) in tetrahydrofuran (1.5 L) was added difluoroacetic anhydride (45.0 g, 258.5 mmol) dropwise at 0° C. After stirring for 2 hours at 0° C., the reaction mixture was quenched with water (1 L) and extracted with ethyl acetate (1 L×3). The combined organic layers were washed with water (800 ml×2) and brine (800 ml×2) and dried over anhydrous sodium sulfate. The solid was filtrated off. The filtrate was concentrated. The residue was triturated with petroleum ether/ethyl acetate (20:1) (200 mL). The solid was collected by filtration and dried under vacuum to give 60.2 g (55% of theory, 93% purity) of the title compound.

LC/MS [Method 40]: Rₜ=1.20 min; MS (ESIpos): m/z=427 [M+H]⁺.

¹H-NMR (300 MHz, CDCl₃): δ [ppm]=7.47-7.74 (m, 15H), 6.98 (t, 1H), 3.75-3.84 (m, 2H), 0.73 (t, 3H).

¹⁹F-NMR (282 MHz, DMSO-d₆): δ [ppm]=−126.55 (d, 2F).

### Intermediate 460A

### Ethyl 4,4-difluorobut-2-ynoate

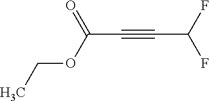

Into a 250-mL round-bottom flask, was placed ethyl 4,4-difluoro-2-[(2E,4Z)-hepta-2,4,6-trien-3-yl(diphenyl)phosphoranylidene]-3-oxobutanoate (Intermediate 459A, 50.0 g, 105.1 mmol, 93% purity). It was thermolyzed heating by sand bath under reduced pressure (˜23 torr) with water Circulating Multi-purpose Vacuum Pump. Once the distillation pot reached to 130° C., the solid phosphorane began to melt and evolution of acetylene. The mixture was heated to 145˜260° C. and the acetylene was collected in the ethanol-dry ice bath to give 13.2 g (59% of theory, 70% purity) of the title compound.

¹H-NMR (400 MHz, CDCl₃): δ [ppm]=6.30 (t, 1H), 4.32 (q, 2H), 1.36 (t, 3H).

¹⁹F-NMR (376 MHz, DMSO-d₆): δ [ppm]=−109.86 (s, 2F).

### Intermediate 461A

### Diethyl 4-(difluoromethyl)-1H-pyrazole-3,5-dicarboxylate

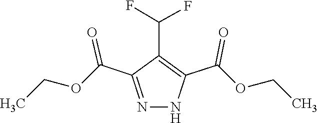 [SMILES: CCOC(=O)c1n[nH]c(C(=O)OCC)c1C(F)F]

To a solution of ethyl 4,4-difluorobut-2-ynoate (Intermediate 460A, 13.0 g, 61.44 mmol, 70% purity) in diethyl ether (150 ml) was added ethyl diazoacetate (7.0 g, 61.4 mmol) under nitrogen atmosphere. After stirring overnight at RT, the reaction mixture was concentrated. The residue was suspended in ethyl acetate/n-hexane (100 ml) (1/20) and stirred for 30 minutes at 0° C. The solid was collected by filtration to give 10.5 g (64% of theory, 99% purity) of the title compound.

LC/MS [Method 43]: Rₜ=1.41 min; MS (ESIpos): m/z=525 [2M+H]⁺.

¹H-NMR (300 MHz, DMSO-d₆): δ [ppm]=15.01 (s, 1H), 7.39 (t, 1H), 4.35 (s, 4H), 1.32 (t, 6H).

¹⁹F-NMR (282 MHz, DMSO-d₆): δ [ppm]=−109.42 (s, 2F).

### Intermediate 462A

### Diethyl 1-[2-(4-chloro-3-fluorophenyl)-2-oxoethyl]-4-(difluoromethyl)-1H-pyrazole-3,5-dicarboxylate

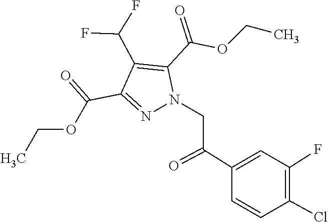 [SMILES: CCOC(=O)c1nn(CC(=O)c2ccc(Cl)c(F)c2)c(C(=O)OCC)c1C(F)F]

To a solution of diethyl 4-(difluoromethyl)-1H-pyrazole-3,5-dicarboxylate (Intermediate 461A, 250.0 mg, 0.96 mmol) and 2-bromo-1-(4-chloro-3-fluorophenyl)ethanone (Intermediate 255A, 287.7 mg, 1.1 mmol) in acetone (8.4 ml) was added potassium carbonate (329.4 mg, 2.4 mmol). The resulting mixture was stirred at RT for 2 h. After filtering off the solids, the filter cake was washed with acetone. The filtrate was evaporated under reduced pressure to give 358.0 mg (79% of theory, 91% purity) of the title compound.

LC/MS [Method 3]: Rₜ=2.25 min; MS (ESIpos): m/z=433 [M+H]⁺.

### Intermediate 463A

### Ethyl 6-(4-chloro-3-fluorophenyl)-3-(difluoromethyl)-4-oxo-4,5-dihydropyrazolo[1,5-a]pyrazine-2-carboxylate

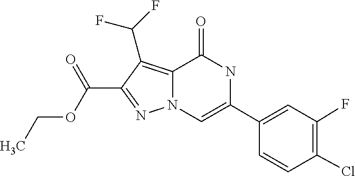

To a solution of diethyl 1-[2-(4-chloro-3-fluorophenyl)-2-oxoethyl]-4-(difluoromethyl)-1H-pyrazole-3,5-dicarboxylate (Intermediate 462A, 358.0 mg, 0.75 mmol, 91% purity) in acetic acid (6.3 ml) was added ammonium acetate (580.9 mg, 7.5 mmol). The resulting mixture was heated overnight at 110° C. After cooling to RT, the reaction mixture was quenched with water. The solid was collected by filtration, washed with water and dried in vacuum to give 236.9 mg (73% of theory, 90% purity) of the title compound.

LC/MS [Method 11]: Rₜ=0.97 min; MS (ESIpos): m/z=384 [M−H]⁻.

### Intermediate 464A

### 6-(4-Chloro-3-fluorophenyl)-3-(difluoromethyl)-4-oxo-4,5-dihydropyrazolo[1,5-a]pyrazine-2-carboxylic acid

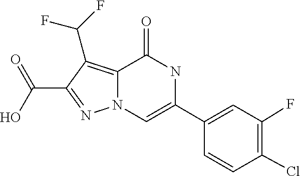

To a mixture of ethyl 6-(4-chloro-3-fluorophenyl)-3-(difluoromethyl)-4-oxo-4,5-dihydropyrazolo-[1,5-a]pyrazine-2-carboxylate (Intermediate 463A, 211.9 mg, 0.55 mmol, 90% purity) in ethanol (15.0 ml) was added a solution 1N of sodium hydroxide (2.7 ml). After stirring at RT for 15 min, the reaction mixture was concentrated under reduced pressure. Then the rest of mixture was diluted with water and acidified with 4N hydrochloric acid. The product was collected by filtration, washed with water and dried in high vacuum to give 169.0 mg (66% of theory, 77% purity) of the title compound.

LC/MS [Method 3]: Rₜ=1.35 min; MS (ESIpos): m/z=356 [M−H]⁻.

### Intermediate 465A

### Ethyl (2Z)-5,5,5-trifluoro-3-{[(trifluoromethyl)sulfonyl]oxy}pent-2-enoate

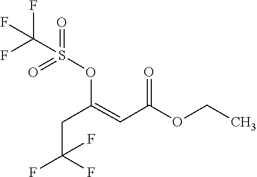 [SMILES: CCOC(=O)/C=C(/CC(F)(F)F)OS(=O)(=O)C(F)(F)F]

To a stirred solution of ethyl 5,5,5-trifluoro-3-oxopentanoate (1.0 g, 5.0 mmol) in toluene (31.5 ml) was added a solution of lithium hydroxide (906.5 mg, 37.9 mmol) in water (9.4 ml) at 0° C. After stirring for 10 min at 10° C., trifluoromethanesulfonic anhydride (1.7 ml, 10.1 mmol) was added dropwise. After stirring overnight at RT, the reaction mixture was quenched with water (80 ml) and extracted with ethyl acetate (3×80 ml). The organic phase was washed with brine and dried over anhydrous sodium sulfate. The solid was filtered off. The filtrate was concentrated under reduced pressure to give 886.0 mg (53% of theory, 95% purity) of the title compound.

¹H-NMR (400 MHz, DMSO-d₆): δ [ppm]=6.64 (s, 1H), 4.23-4.17 (m, 2H), 3.77-3.69 (m, 2H), 1.24 (t, 3H).

### Intermediate 466A

### Diethyl 4-(2,2,2-trifluoroethyl)-1H-pyrazole-3,5-dicarboxylate

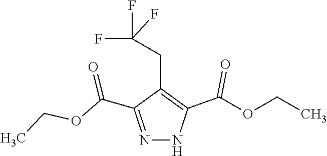 [SMILES: CCOC(=O)c1n[nH]c(C(=O)OCC)c1CC(F)(F)F]

To a stirred solution of ethyl (2Z)-5,5,5-trifluoro-3-{[(trifluoromethyl)sulfonyl]oxy}pent-2-enoate (Intermediate 465A, 500.0 mg, 1.5 mmol, 95% purity) in N,N-dimethylformamide (4.5 ml) were added ethyl diazoacetate (0.24 ml, 2.3 mmol) and 4-methylmorpholine (0.3 ml, 3.1 mmol) followed by tetrakis(triphenylphosphine)palladium(0) (87.5 mg, 0.07 mmol) at RT. The resulting mixture was stirred for 5 minutes at RT under Argon and overnight at 60° C. The reaction mixture was quenched with water (15 ml) and extracted with three times with ethyl acetate. The organic phase was washed with brine and dried over anhydrous sodium sulfate. The solid was filtered off. The filtrate was concentrated under reduced pressure to give 639 mg (>100% of theory, 47% purity) of the title compound.

LC/MS [Method 11]: Rₜ=0.87 min; MS (ESIpos): m/z=279 [M+H]⁺.

### Intermediate 467A

### Diethyl 1-[2-(3-fluoro-4-methylphenyl)-2-oxoethyl]-4-(2,2,2-trifluoroethyl)-1H-pyrazole-3,5-dicarboxylate

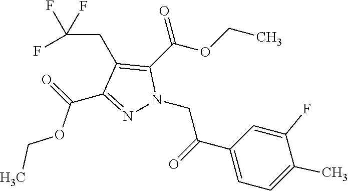 [SMILES: CCOC(=O)c1nn(CC(=O)c2ccc(C)c(F)c2)c(C(=O)OCC)c1CC(F)(F)F]

To a solution of diethyl 4-(2,2,2-trifluoroethyl)-1H-pyrazole-3,5-dicarboxylate (Intermediate 466A, 445.5 mg, 1.5mmol, 47% purity) and 2-bromo-1-(3-fluoro-4-methylphenyl)ethanone (419.8 mg, 1.8 mmol) in acetone (13.3 ml) was added potassium carbonate (523.1 mg, 3.8 mmol). The resulting mixture was stirred for 2 h at RT. After filtering off the solids, the filter cake was washed with ethyl acetate and the filtrate was concentrated under reduced pressure. The residue was purified by preparative HPLC (Method P16) to give 38.0 mg (4% of theory, 62% purity) of the title compound.

LC/MS [Method 11]: Rₜ=4.10 min; MS (ESIpos): m/z=445 [M+H]⁺.

### Intermediate 468A

### Ethyl 6-(3-fluoro-4-methylphenyl)-4-oxo-3-(2,2,2-trifluoroethyl)-4,5-dihydropyrazolo[1,5-a]-pyrazine-2-carboxylate

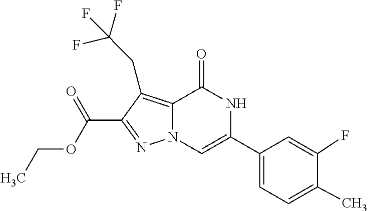 [SMILES: CCOC(=O)c1nn2cc(-c3ccc(C)c(F)c3)[nH]c(=O)c2c1CC(F)(F)F]

To a solution of diethyl 1-[2-(3-fluoro-4-methylphenyl)-2-oxoethyl]-4-(2,2,2-trifluoroethyl)-1H-pyrazole-3,5-dicarboxylate (Intermediate 467A, 62 mg, 0.14 mmol) in acetic acid (0.7 ml) was added ammonium acetate (107.5 mg, 1.4 mmol). The resulting mixture was heated for 7 days at 110° C. After cooled to RT, the reaction mixture was quenched with water. The solid was collected by filtration, washed with water and dried in vacuum to give 35.0 mg (63% of theory, 100% purity) of the title compound.

LC/MS [Method 3]: Rₜ=2.05 min; MS (ESIpos): m/z=398 [M+H]⁺.

### Intermediate 469A

### 6-(3-Fluoro-4-methylphenyl)-4-oxo-3-(2,2,2-trifluoroethyl)-4,5-dihydropyrazolo[1 ,5-a]pyrazine-2-carboxylic acid

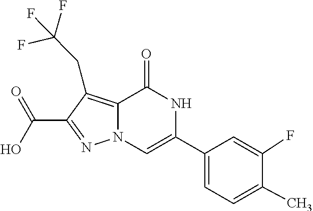 [SMILES: Cc1ccc(-c2cn3nc(C(=O)O)c(CC(F)(F)F)c3c(=O)[nH]2)cc1F]

To a mixture of ethyl 6-(3-fluoro-4-methylphenyl)-4-oxo-3-(2,2,2-trifluoroethyl)-4,5-dihydropyrazolo[1,5-a]pyrazine-2-carboxylate (Intermediate 468A, 35.0 mg, 0.09 mmol) in a mixture THF/methanol (1.5 ml, 5/1) was added a solution of 1M of lithium hydroxide (0.44 ml). After stirring at RT for 3 hours, the reaction mixture was concentrated under reduced and acidified with 2N hydrochloric acid. The product was collected by filtration, washed with water and dried in air to give 26.0 mg (73% of theory, 91% purity) of the title compound.

LC/MS [Method 3]: Rₜ=1.56 min; MS (ESIpos): m/z=370 [M+H]⁺.

### Intermediate 470A

### Diethyl 1-[2-(5,6-dimethylpyridin-2-yl)-2-oxoethyl]-4-methyl-1H-pyrazole-3,5-dicarboxylate

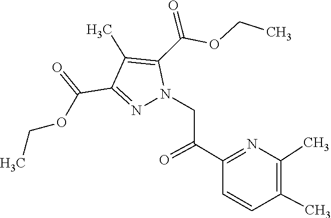 [SMILES: CCOC(=O)c1nn(CC(=O)c2ccc(C)c(C)n2)c(C(=O)OCC)c1C]

To a solution of diethyl 4-methyl-1H-pyrazole-3,5-dicarboxylate (Intermediate 54A, 441.2 mg, 1.9 mmol) and 2-bromo-1-(5,6-dimethylpyridin-2-yl)ethanone (Intermediate 82A, 723.1 mg, 2.3 mmol) in acetone (17.1 ml) was added potassium carbonate (673.7 mg, 4.9 mmol). The resulting mixture was stirred overnight at RT. Afterwards, the reaction was stirred overnight at 50° C. After filtering off the solids, the filter cake was washed with acetone. The filtrate was evaporated under reduced pressure to give 2.8 g (>100% of theory, 63% purity) of the title compound.

LC/MS [Method 3]: Rₜ=2.22 min; MS (ESIpos): m/z=374 [M+H]⁺.

### Intermediate 471A

### Ethyl 6-(5,6-dimethylpyridin-2-yl)-3-methyl-4-oxo-4,5-dihydropyrazolo[1,5-a]pyrazine-2-carboxylate

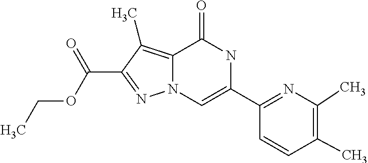

To a solution of diethyl 1-[2-(5,6-dimethylpyridin-2-yl)-2-oxoethyl]-4-methyl-1H-pyrazole-3,5-dicarboxylate (Intermediate 470A, 728.2 mg, 1.9 mmol, 63% purity) in acetic acid (38.9 ml) was added ammonium acetate (6.0 g, 78.0 mmol). The resulting mixture was heated for 2 days at 110° C. After cooling to RT, the reaction mixture was quenched with water. The solid was collected by filtration and washed with water. The pH was adjusted to 5-6 with a solution 1N of sodium hydroxide, and dried in vacuum. The residue was purified by preparative HPLC (Method P16) to give 491.0 mg (54% of theory, 70% purity) of the title compound.

LC/MS [Method 3]: Rₜ=1.95 min; MS (ESIpos): m/z=327 [M+H]⁺.

### Intermediate 472A

### 6-(5,6-Dimethylpyridin-2-yl)-3-methyl-4-oxo-4,5-dihydropyrazolo[1,5-a]pyrazine-2-carboxylic acid

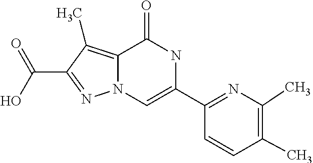

To a mixture of ethyl 6-(5,6-dimethylpyridin-2-yl)-3-methyl-4-oxo-4,5-dihydropyrazolo[1,5-a]-pyrazine-2-carboxylate (Intermediate 472A, 136.0 mg, 0.42 mmol, 70% purity) in a mixture THF/methanol (3.3 ml, 5/1) was added a solution 1M of lithium hydroxide (2.1 ml). After stirring overnight at RT, the reaction mixture was concentrated under reduced pressure. Then the rest of mixture was diluted with water and acidified with formic acid. The product was collected by filtration, washed with water and dried in high vacuum to give 135.0 mg (84% of theory, 77% purity) of the title compound.

LC/MS [Method 3]: Rₜ=1.38 min; MS (ESIpos): m/z=299 [M+H]⁺.

### Intermediate 473A

### Diethyl 1-[2-(3,4-dichlorophenyl)-2-oxoethyl]-1H-pyrazole-3,5-dicarboxylate

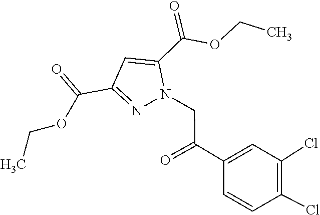 [SMILES: CCOC(=O)c1cc(C(=O)OCC)n(CC(=O)c2ccc(Cl)c(Cl)c2)n1]

To a solution of diethyl 1H-pyrazole-3,5-dicarboxylate (1.00 g, 4.71 mmol) in acetone (40 ml) were added 2-bromo-1-(3,4-dichlorophenyl)ethanone (1.51 g, 5.65 mmol) and potassium carbonate (1.63 g, 11.8 mmol). The mixture was stirred at RT for 1 h. After filtering off the solids, the filtrate was concentrated and the residue dried in vacuo to give 2.18 g (quant., 90% purity) of the title compound. This compound was used without further purification.

LC/MS [Method 7]: Rₜ=1.17 min; MS (ESIpos): m/z=399 [M+H]⁺.

### Intermediate 474A

### Ethyl 6-(3,4-dichlorophenyl)-4-oxo-4,5-dihydropyrazolo[1,5-a]pyrazine-2-carboxylate

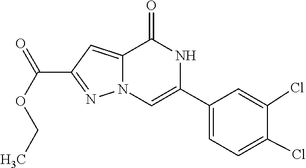 [SMILES: CCOC(=O)c1cc2c(=O)[nH]c(-c3ccc(Cl)c(Cl)c3)cn2n1]

To a solution of diethyl 1-[2-(3,4-dichlorophenyl)-2-oxoethyl]-1H-pyrazole-3,5-dicarboxylate (Intermediate 473A, 2.18 g, 4.92 mmol, 90% purity) in acetic acid (98 ml) was added ammonium acetate (15.2 g, 197 mmol). After refluxing overnight, the reaction mixture was cooled to RT and diluted with 100 ml water. The precipitate was collected by filtration, washed with water and dried in vacuo to give 1.72 g (99% of theory, 100% purity) of the title compound.

LC/MS [Method 3]: Rₜ=1.79 min; MS (ESIpos): m/z=352 [M+H]⁺.

### Intermediate 475A

### 6-(3,4-dichlorophenyl)-4-oxo-4,5-dihydropyrazolo[1,5-a]pyrazine-2-carboxylic acid

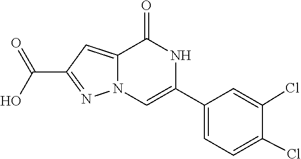 [SMILES: O=C(O)c1cc2c(=O)[nH]c(-c3ccc(Cl)c(Cl)c3)cn2n1]

To a suspension of ethyl 6-(3,4-dichlorophenyl)-4-oxo-4,5-dihydropyrazolo[1,5-a]pyrazine-2-carboxylate (Intermediate 474A, 1.72 g, 4.89 mmol) in THF (30 ml) and methanol (6 ml) was added 1 M lithium hydroxide solution (24.4 ml, 24.4 mmol). After stirring at RT for 0.5 h, volatiles were partly removed under reduced pressure, the remaining mixture was adjusted to pH 3 with 1 M hydrochloric acid under ice-bath cooling. The solid was collected by filtration, washed with water and dried in vacuo to give 1.63 g (99% of theory, purity 97%) of the title compound.

LC/MS [Method 7]: Rₜ=0.77 min; MS (ESIpos): m/z=323.9 [M+H]⁺.

¹H-NMR (500 MHz, DMSO-d₆): δ [ppm]=13.30 (s, 1H), 11.80 (s, 1H), 8.29 (s, 1H), 8.08 (s, 1H), 7.79-7.75 (m, 2H), 7.37 (s, 1H).

### Intermediate 476A

### Diethyl1-[2-(3-fluoro-4-methylphenyl)-2-oxoethyl]-1H-pyrazole-3,5-dicarboxylate

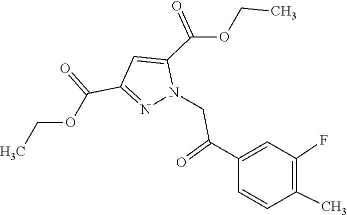 [SMILES: CCOC(=O)c1cc(C(=O)OCC)n(CC(=O)c2ccc(C)c(F)c2)n1]

To a solution of diethyl 1H-pyrazole-3,5-dicarboxylate (0.25 g, 1.18 mmol) in acetone (10 ml) were added 2-bromo-1-(3-fluoro-4-methylphenyl)ethanone (0.33 g, 1.41 mmol) and potassium carbonate (0.41 g, 2.95 mmol). The mixture was stirred at RT over night. After filtering off the solids, the filtrate was concentrated and the residue dried in vacuo to give 0.48 g (99% of theory, 87% purity) of the title compound. This compound was used without further purification.

LC/MS [Method 3]: Rₜ=2.07 min; MS (ESIpos): m/z=363 [M+H]⁺.

### Intermediate 477A

### Ethyl 6-(3-fluoro-4-methylphenyl)-4-oxo-4,5-dihydropyrazolo[1,5-a]pyrazine-2-carboxylate

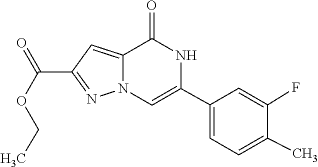 [SMILES: CCOC(=O)c1cc2c(=O)[nH]c(-c3ccc(C)c(F)c3)cn2n1]

To a solution of diethyl1-[2-(3-fluoro-4-methylphenyl)-2-oxoethyl]-1H-pyrazole-3,5-dicarboxylate (Intermediate 476A, 0.47 g, 1.17 mmol, 87% purity) in acetic acid (23 ml) was added ammonium acetate (3.61 g, 46.8 mmol). After refluxing overnight, the reaction mixture was cooled to RT and diluted with 100 ml water. The precipitate was collected by filtration, washed with water and dried in vacuo to give 0.25 g (53% of theory, 78% purity) of the title compound.

LC/MS [Method 3]: Rₜ=1.65 min; MS (ESIpos): m/z=316 [M+H]⁺.

### Intermediate 478A

### 6-(3-Fluoro-4-methylphenyl)-4-oxo-4,5-dihydropyrazolo[1,5-a]pyrazine-2-carboxylic acid

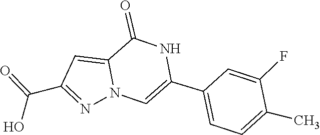 [SMILES: Cc1ccc(-c2cn3nc(C(=O)O)cc3c(=O)[nH]2)cc1F]

To a suspension of ethyl 6-(3-fluoro-4-methylphenyl)-4-oxo-4,5-dihydropyrazolo[1,5-a]pyrazine-2-carboxylate (Intermediate 477A, 0.25 g, 0.62 mmol, 78% purity) in 4,7 ml THF/methanol (5:1) was added 1 M lithium hydroxide solution (3.1 ml, 3.1 mmol). After stirring at RT for 0.5 h, volatiles were partly removed under reduced pressure, the remaining mixture was adjusted to pH 2 with 2 M hydrochloric acid. The solid was collected by filtration, washed with water and dried in vacuo to give 0.24 g (135% of theory, purity 99%) of the title compound.

LC/MS [Method 3]: Rₜ=1.20 min; MS (ESIpos): m/z=287.2 [M+H]⁺.

¹H-NMR (600 MHz, DMSO-d₆): δ [ppm]=13.30 (s, br 1H), 11.72 (s, 1H), 8.20 (s, 1H), 7.61 (d, 1H), 7.54 (d, 1H), 7.41 (t, 1H), 7.35 (s, 1H), 2.29 (s, 3H).

### Intermediate 479A

### Diethyl 1-[2-(2-fluoro-3,4-dimethylphenyl)-2-oxoethyl]-1H-pyrazole-3,5-dicarboxylate

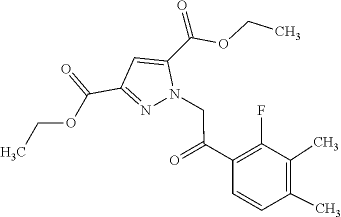 [SMILES: CCOC(=O)c1cc(C(=O)OCC)n(CC(=O)c2ccc(C)c(C)c2F)n1]

To a solution of diethyl 1H-pyrazole-3,5-dicarboxylate (0.51 g, 2.40 mmol) in acetone (20 ml) were added Intermediate 251A (0.92 g, 2.64 mmol, 70% purity) and potassium carbonate (0.83 g, 6.00 mmol). The mixture was stirred at RT for 1h. After filtering off the solids, the filtrate was concentrated and the residue dried in vacuo to give 1.15 g (95% of theory, 75% purity) of the title compound. This compound was used without further purification.

LC/MS [Method 3]: Rₜ=2.23 min; MS (ESIpos): m/z=377 [M+H]⁺.

### Intermediate 480A

### Ethyl 6-(2-fluoro-3,4-dimethylphenyl)-4-oxo-4,5-dihydropyrazolo[1,5-a]pyrazine-2-carboxylate

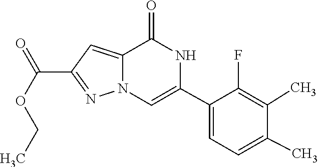 [SMILES: CCOC(=O)c1cc2c(=O)[nH]c(-c3ccc(C)c(C)c3F)cn2n1]

To a solution of diethyl 1-[2-(2-fluoro-3,4-dimethylphenyl)-2-oxoethyl]-1H-pyrazole-3,5-dicarboxylate (Intermediate 479A, 1.15 g, 2.29 mmol, 75% purity) in acetic acid (12 ml) was added ammonium acetate (3.52 g, 45.7 mmol). After refluxing for 72 h the reaction mixture was cooled to RT and diluted with 100 ml water. The precipitate was collected by filtration, washed with water and dried in vacuo to give 0.63 g (58% of theory, 69% purity) of the title compound. This compound was used without further purification.

LC/MS [Method 7]: Rₜ=0.94 min; MS (ESIpos): m/z=330 [M+H]⁺.

### Intermediate 481A

### 6-(2-Fluoro-3,4-dimethylphenyl)-4-oxo-4,5-dihydropyrazolo[1,5-a]pyrazine-2-carboxylic acid

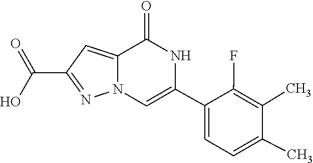 [SMILES: Cc1ccc(-c2cn3nc(C(=O)O)cc3c(=O)[nH]2)c(F)c1C]

To a suspension of ethyl 6-(2-fluoro-3,4-dimethylphenyl)-4-oxo-4,5-dihydropyrazolo[1,5-a]pyrazine-2-carboxylate (Intermediate 480A, 0.63 g, 1.91 mmol) in 14.5 ml THF/methanol (5:1) was added 1 M lithium hydroxide solution (9.6 ml, 9.6 mmol). After stirring at RT for 1 h, volatiles were partly removed under reduced pressure, the remaining mixture was adjusted to pH 3 with 1 M hydrochloric acid. The solid was collected by filtration, washed with water and dried in vacuo to give 0.56 g (97% of theory) of the title compound.

LC/MS [Method 3]: Rₜ=1.27 min; MS (ESIpos): m/z=302 [M+H]⁺.

¹H-NMR (400 MHz, DMSO-d₆): δ [ppm]=13.30 (s, br 1H), 11.71 (s, 1H), 7.88 (s, 1H), 7.33 (m, 2H), 7.13 (d, 1H), 2.33 (s, 3H), 2.20 (s, 3H).

### Intermediate 482A

### 2-Bromo-1-(4-fluoro-3-methylphenyl)ethanone

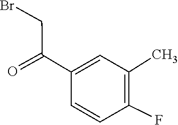

To a solution of 1-(4-fluoro-3-methylphenyl)ethanone (2.00 g, 13.1 mmol) in THF (10 ml) was added dropwise a solution of Phenyltrimethylammoniumtribromid (6.11 g, 15.7 mmol) in THF (10 ml). The mixture was stirred at 50° C. for 1h. After filtering off the solids, the filtrate was concentrated and the residue dried in vacuo to give 4.22 g (98% of theory, 70% purity) of the title compound. This compound was used without further purification.

LC/MS [Method 3]: Rₜ=1.85 min; MS (ESIpos): m/z=230.9 [M+H]⁺.

### Intermediate 483A

### Diethyl1-[2-(4-fluoro-3-methylphenyl)-2-oxoethyl]-4-(trifluoromethyl)-1H-pyrazole-3,5-dicarboxylate

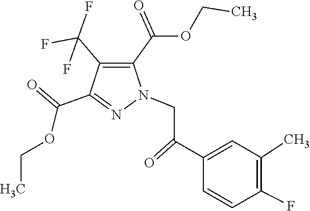 [SMILES: CCOC(=O)c1nn(CC(=O)c2ccc(F)c(C)c2)c(C(=O)OCC)c1C(F)(F)F]

To a solution of diethyl 4-(trifluoromethyl)-1H-pyrazole-3,5-dicarboxylate (Intermediate 32A, 1.00 g, 3.56 mmol) in acetone (31 ml) were added Intermediate 482A (1.40 g, 4.27 mmol, 70% purity) and potassium carbonate (1.23 g, 8.89 mmol). The mixture was stirred at RT for 1 h. After filtering off the solids, the filtrate was concentrated and the residue dried in vacuo to give 1.48 g (97% of theory) of the title compound.

LC/MS [Method 3]: Rₜ=2.29 min; MS (ESIpos): m/z=431 [M+H]⁺.

### Intermediate 484A

### Ethyl 6-(4-fluoro-3-methylphenyl)-4-oxo-3-(trifluoromethyl)-4,5-dihydropyrazolo[1,5-a]pyrazine-2-carboxylate

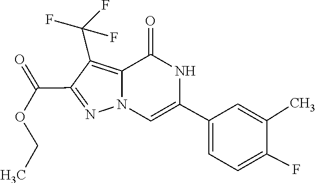 [SMILES: CCOC(=O)c1nn2cc(-c3ccc(F)c(C)c3)[nH]c(=O)c2c1C(F)(F)F]

To a solution of diethyl1-[2-(4-fluoro-3-methylphenyl)-2-oxoethyl]-4-(trifluoromethyl)-1H-pyrazole-3,5-dicarboxylat (Intermediate483A, 1.48 g, 3.44 mmol) in acetic acid (15 ml) was added ammonium acetate (2.65 g, 34.4 mmol). After refluxing for 16 h the reaction mixture was cooled to RT and diluted with 100 ml water. The precipitate was collected by filtration, washed with acetone and dried in vacuo to give 0.50 g (38% of theory) of the title compound.

LC/MS [Method 3]: Rₜ=1.98 min; MS (ESIpos): m/z=384 [M+H]⁺.

### Intermediate 485A

### 6-(4-Fluoro-3-methylphenyl)-4-oxo-3-(trifluoromethyl)-4,5-dihydropyrazolo[1,5-a]pyrazine-2-carboxylic acid

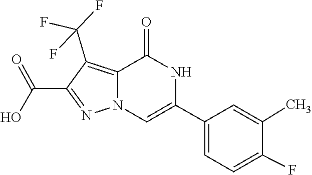 [SMILES: Cc1cc(-c2cn3nc(C(=O)O)c(C(F)(F)F)c3c(=O)[nH]2)ccc1F]

To a suspension of ethyl 6-(4-fluoro-3-methylphenyl)-4-oxo-3-(trifluoromethyl)-4,5-dihydropyrazolo[1,5-a]pyrazine-2-carboxylate (Intermediate 484A, 503 mg, 1.31 mmol) in 9.93 ml THF/methanol (5:1) was added 1 M lithium hydroxide solution (6.56 ml, 6.56 mmol). After stirring at RT for 1 h, volatiles were partly removed under reduced pressure, the remaining mixture was adjusted to pH 3 with 1 M hydrochloric acid. The solid was collected by filtration, washed with water and dried in vacuo to give 0.45 g (97% of theory) of the title compound.

LC/MS [Method 7]: Rₜ=0.77 min; MS (ESIpos): m/z=355 [M+H]⁺.

¹H-NMR (400 MHz, DMSO-d₆): δ [ppm]=13.9 (s, br 1H), 12.06 (s, 1H), 8.18 (s, 1H), 7.74 (dd, 1H), 7.63 (m, 1H), 7.29 (t, 1H), 2.30 (s, 3H).

### Intermediate 486A

### Diethyl 1-[2-(4-chloro-3-fluorophenyl)-2-oxoethyl]-4-(trifluoromethyl)-1H-pyrazole-3,5-dicarboxylate

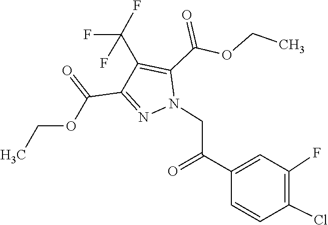 [SMILES: CCOC(=O)c1nn(CC(=O)c2ccc(Cl)c(F)c2)c(C(=O)OCC)c1C(F)(F)F]

To a solution of diethyl 4-(trifluoromethyl)-1H-pyrazole-3,5-dicarboxylate (Intermediate 32A, 0.88 g, 3.14 mmol) in acetone (28 ml) were added 2-bromo-1-(4-chloro-3-fluorophenyl)ethanone (Intermediate 255A, 1.20 g, 3.77 mmol, 79% purity) and potassium carbonate (1.08 g, 7.85 mmol). The mixture was stirred at RT for 1 h. After filtering off the solids, the filtrate was concentrated and the residue dried in vacuo to give 1.62 g (96% of theory, 84% purity) of the title compound. This compound was used without further purification.

LC/MS [Method 3]: Rₜ=2.33 min; MS (ESIpos): m/z=451 [M+H]⁺.

### Intermediate 487A

### Ethyl 6-(4-chloro-3-fluorophenyl)-4-oxo-3-(trifluoromethyl)-4,5-dihydropyrazolo[1,5-a]pyrazine-2-carboxylate

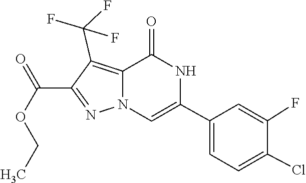 [SMILES: CCOC(=O)c1nn2cc(-c3ccc(Cl)c(F)c3)[nH]c(=O)c2c1C(F)(F)F]

To a solution of diethyl 1-[2-(4-chloro-3-fluorophenyl)-2-oxoethyl]-4-(trifluoromethyl)-1H-pyrazole-3,5-dicarbo-xylate (Intermediate 486A, 1.62 g, 3.03 mmol, 84% purity) in acetic acid (27 ml) was added ammonium acetate (4.67 g, 60.6 mmol). After refluxing for 72 h the reaction mixture was cooled to RT and diluted with 100 ml water. The precipitate was collected by filtration and dried in vacuo to give 0.99 g (80% of theory) of the title compound.

LC/MS [Method 3]: Rₜ=1.98 min; MS (ESIpos): m/z=404 [M+H]⁺.

### Intermediate 488A

### 6-(4-Chloro-3-fluorophenyl)-4-oxo-3-(trifluoromethyl)-4,5-dihydropyrazolo[1,5-a]pyrazine-2-carboxylic acid

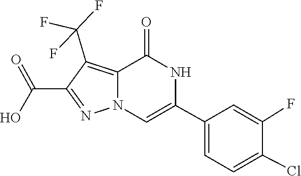 [SMILES: O=C(O)c1nn2cc(-c3ccc(Cl)c(F)c3)[nH]c(=O)c2c1C(F)(F)F]

To a suspension of ethyl 6-(4-chloro-3-fluorophenyl)-4-oxo-3-(trifluoromethyl)-4,5-dihydropyrazolo[1,5-a]pyrazine-2-carboxylate (Intermediate 487A, 1.00 g, 2.41 mmol) in 18.3 ml THF/methanol (5:1) was added 1 M lithium hydroxide solution (12.1 ml, 12.1 mmol). After stirring at RT for 1 h, volatiles were partly removed under reduced pressure, the remaining mixture was adjusted to pH 3 with 1 M hydrochloric acid. The solid was collected by filtration, washed with water and dried in vacuo to give 0.90 g (97% of theory) of the title compound.

LC/MS [Method 3]: Rₜ=1.32 min; MS (ESIpos): m/z=375 [M+H]⁺.

1H-NMR (400 MHz, DMSO-d6): δ [ppm]=14.03 (s, br 1H), 12.16 (s, 1H), 8.36 (s, 1H), 7.89 (dd, 1H), 7.76 (t, 1H), 7.67 (dd, 1H).

### Intermediate 489A

### Diethyl 4-cyclopropyl-1-[2-(4-fluoro-3-methylphenyl)-2-oxoethyl]-1H-pyrazole-3,5-dicarboxylate

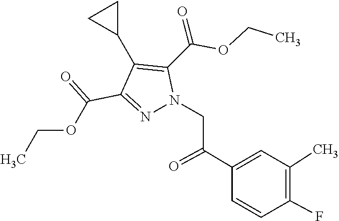 [SMILES: CCOC(=O)c1nn(CC(=O)c2ccc(F)c(C)c2)c(C(=O)OCC)c1C1CC1]

To a solution of diethyl 4-cyclopropyl-1H-pyrazole-3,5-dicarboxylate (Intermediate 187A, 1.79 g, 7.11 mmol) in acetone (62 ml) were added Intermediate 482A (2.80 g, 8.53 mmol, 70% purity) and potassium carbonate (2.46 g, 17.8 mmol). The mixture was stirred at RT for 1 h. After filtering off the solids, the filtrate was concentrated and the residue dried in vacuo to give 4.10 g (99% of theory, 70% purity) of the title compound. This compound was used without further purification.

LC/MS [Method 3]: Rₜ=2.26 min; MS (ESIpos): m/z=403 [M+H]⁺.

### Intermediate 490A

### Ethyl 3-cyclopropyl-6-(4-fluoro-3-methylphenyl)-4-oxo-4,5-dihydropyrazolo[1,5-a]pyrazine-2-carboxylate

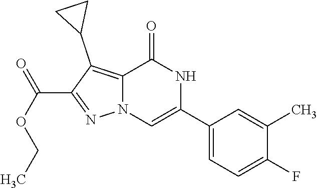 [SMILES: CCOC(=O)c1nn2cc(-c3ccc(F)c(C)c3)[nH]c(=O)c2c1C1CC1]

To a solution of diethyl 4-cyclopropyl-1-[2-(4-fluoro-3-methylphenyl)-2-oxoethyl]-1H-pyrazole-3,5-dicarboxylate (Intermediate 489A, 4.10 g, 7.11 mmol, 70% purity) in acetic acid (31 ml) was added ammonium acetate (5.46 g, 70.1 mmol). After refluxing for 16 h the reaction mixture was cooled to RT and diluted with 100 ml water. The precipitate was collected by filtration, washed with Aceton and dried in vacuo to give 1.23 g (49% of theory) of the title compound. LC/MS [Method 3]: Rₜ=2.00 min; MS (ESIpos): m/z=356 [M+H]⁺.

### Intermediate 491A

### 3-Cyclopropyl-6-(4-fluoro-3-methylphenyl)-4-oxo-4,5-dihydropyrazolo[1,5-a]pyrazine-2-carboxylic acid

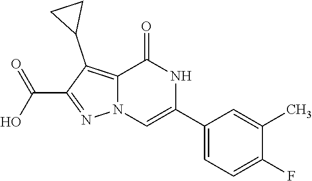 [SMILES: Cc1cc(-c2cn3nc(C(=O)O)c(C4CC4)c3c(=O)[nH]2)ccc1F]

To a suspension of ethyl 3-cyclopropyl-6-(4-fluoro-3-methylphenyl)-4-oxo-4,5-dihydropyrazolo-[1,5-a]pyrazine-2-carboxylate (Intermediate 490A, 1.23 g, 3.45 mmol) in 26 ml THF/methanol (5:1) was added 1 M lithium hydroxide solution (17.3 ml, 17.3 mmol). After stirring at RT for 1 h, volatiles were partly removed under reduced pressure, the remaining mixture was adjusted to pH 3 with 1 M hydrochloric acid. The solid was collected by filtration, washed with water and dried in vacuo to give 1.10 g (95% of theory) of the title compound.

LC/MS [Method 7]: Rₜ=0.84 min; MS (ESIpos): m/z=328 [M+H]⁺.

¹H-NMR (400 MHz, DMSO-d₆): δ [ppm]=13.1 (s, br 1H), 11.41 (s, 1H), 7.96 (s, 1H), 7.71 (dd, 1H), 7.60 (m, 1H), 7.26 (t, 1H), 2.74 (m, 1H), 3.21 (m, 3H), 1.26 (m, 2H), 0.92 (m, 2H).

### Intermediate 492A

### Diethyl 1-[2-(1-methyl-1H-benzimidazol-5-yl)-2-oxoethyl]-4-(trifluoromethyl)-1H-pyrazole-3,5-dicarboxylate

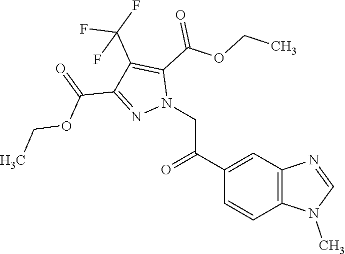 [SMILES: CCOC(=O)c1nn(CC(=O)c2ccc3c(c2)ncn3C)c(C(=O)OCC)c1C(F)(F)F]

To a solution of diethyl 4-(trifluoromethyl)-1H-pyrazole-3,5-dicarboxylate (Intermediate 32A, 0.29 g, 1.04 mmol) in acetone (9 ml) were added 2-bromo-1-(1-methyl-1H-benzimidazol-5-yl)ethanone hydrobromide (0.41 g, 1.24 mmol) and potassium carbonate (0.36 g, 2.59 mmol). The mixture was stirred at RT for 1 h. After filtering off the solids, the filtrate was concentrated and the residue dried in vacuo to give 0.49 g (97% of theory, 91% purity) of the title compound. This compound was used without further purification.

LC/MS [Method 3]: Rₜ=1.77 min; MS (ESIpos): m/z=453 [M+H]⁺.

### Intermediate 493A

### Ethyl 6-(1-methyl-1H-benzimidazol-5-yl)-4-oxo-3-(trifluoromethyl)-4,5-dihydropyrazolo[1,5-a]pyrazine-2-carboxylate

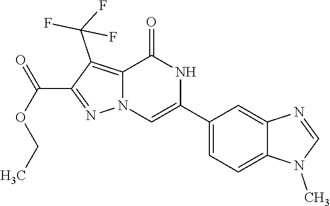 [SMILES: CCOC(=O)c1nn2cc(-c3ccc4c(c3)ncn4C)[nH]c(=O)c2c1C(F)(F)F]

To a solution of diethyl 1-[2-(1-methyl-1H-benzimidazol-5-yl)-2-oxoethyl]-4-(trifluoromethyl)-1H-pyrazole-3,5-dicarboxylate (Intermediate 492A, 0.49 g, 1.01 mmol, 91% purity) in acetic acid (20 ml) was added ammonium acetate (3.10 g, 40.2 mmol). After refluxing for 4 d more ammonium acetate (0.31 g, 4.02 mmol) was added and the the mixture was stirred at 110° C. for another 16 h. The reaction mixture was cooled to RT and diluted with 100 ml water. The precipitate was collected by filtration, washed with water and dried in vacuo to give 0.26 g (57% of theory, 89 purity) of the title compound.

LC/MS [Method 3]: Rₜ=1.40 min; MS (ESIpos): m/z=406 [M+H]⁺.

### Intermediate 494A

### 6-(1-Methyl-1H-benzimidazol-5-yl)-4-oxo-3-(trifluoromethyl)-4,5-dihydropyrazolo[1,5-a]pyrazine-2-carboxylic acid

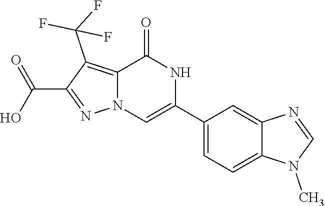 [SMILES: Cn1cnc2cc(-c3cn4nc(C(=O)O)c(C(F)(F)F)c4c(=O)[nH]3)ccc21]

To a suspension of ethyl 6-(1-methyl-1H-benzimidazol-5-yl)-4-oxo-3-(trifluoromethyl)-4,5-dihydropyrazolo[1,5-a]pyrazine-2-carboxylate (Intermediate 493A, 0.26 g, 0.65 mmol) in 4.9 ml THF/methanol (5:1) was added 1 M lithium hydroxide solution (3.25 ml, 3.25 mmol). After stirring at RT for 1 h, volatiles were partly removed under reduced pressure, the remaining mixture was adjusted to pH 3 with 1 M hydrochloric acid. The solid was collected by filtration, washed with water and dried in vacuo to give 0.20 g (81% of theory) of the title compound.

LC/MS [Method 7]: Rₜ=0.56 min; MS (ESIpos): m/z=378 [M+H]⁺.

¹H-NMR (400 MHz, DMSO-d₆): δ [ppm]=14.0 (s, br 1H), 12.16 (s, 1H), 8.56 (s, 1H), 8.24 (s, 1H), 8.12 (s, 1H), 7.79 (d, 1H), 7.74 (d, 1H), 3.93 (s, 3H).

### Intermediate 495A

### Diethyl 1-[2-(1-methyl-1H-benzimidazol-5-yl)-2-oxoethyl]-1H-pyrazole-3,5-dicarboxylate

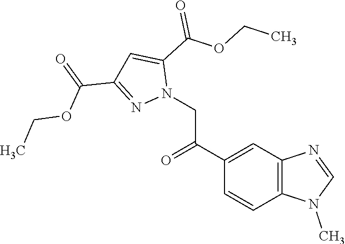 [SMILES: CCOC(=O)c1cc(C(=O)OCC)n(CC(=O)c2ccc3c(c2)ncn3C)n1]

To a solution of diethyl 1H-pyrazole-3,5-dicarboxylate (0.30 g, 1.41 mmol) in acetone (12 ml) were added 2-bromo-1-(1-methyl-1H-benzimidazol-5-yl)ethanone hydrobromide (0.57 g, 1.70 mmol) and potassium carbonate (0.88 g, 6.36 mmol). The mixture was stirred at RT for 1 h. After filtering off the solids, the filtrate was concentrated and the residue dried in vacuo to give 0.57 g (94% of theory, 87% purity) of the title compound. This compound was used without further purification.

LC/MS [Method 7]: Rₜ=0.78 min; MS (ESIpos): m/z=385 [M+H]⁺.

### Intermediate 496A

### Ethyl 6-(1-methyl-1H-benzimidazol-5-yl)-4-oxo-4,5-dihydropyrazolo[1,5-a]pyrazine-2-carboxylate

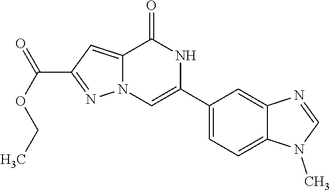 [SMILES: CCOC(=O)c1cc2c(=O)[nH]c(-c3ccc4c(c3)ncn4C)cn2n1]

To a solution of diethyl 1-[2-(1-methyl-1H-benzimidazol-5-yl)-2-oxoethyl]-1H-pyrazole-3,5-dicarboxylate (Intermediate 495A, 0.57 g, 1.33 mmol, 87% purity) in acetic acid (26 ml) was added ammonium acetate (4.10 g, 53.0 mmol). After refluxing for 16 h the reaction mixture was cooled to RT and diluted with 100 ml water. The solvents were distilled off and the residue purified by column chromatography over silica gel with a dichlormethane/methanol gradient (60:1→40:1) to give 2.65 g (>>100% of theory, 100% LC-MS purity) of the title compound. This compound was used without further purification. The maximum theoretical yield was 0.45 g. This amount was used in the following reaction.

LC/MS [Method 7]: Rₜ=0.54 min; MS (ESIpos): m/z=338 [M+H]⁺.

### Intermediate 497A

### 6-(1-Methyl-1H-benzimidazol-5-yl)-4-oxo-4,5-dihydropyrazolo[1,5-a]pyrazine-2-carboxylic acid

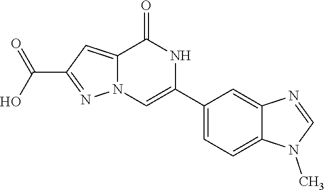 [SMILES: Cn1cnc2cc(-c3cn4nc(C(=O)O)cc4c(=O)[nH]3)ccc21]

To a suspension of ethyl 6-(1-methyl-1H-benzimidazol-5-yl)-4-oxo-4,5-dihydropyrazolo[1,5-a]-pyrazine-2-carboxylate (Intermediate 496A, 0.45 g, 1.33 mmol) in 12.3 ml THF/methanol (5:1) was added 1 M lithium hydroxide solution (6.63 ml, 6.63 mmol). After stirring at RT for 0.5 h, the mixture was acidified with formic acid . The solid was collected by filtration, washed with water and dried in vacuo to give 0.29 g (60% of theory, 85% LC-MS purity) of the title compound. This compound was used without further purification.

LC/MS [Method 7]: Rₜ=0.56 min; MS (ESIpos): m/z=378 [M+H]⁺.

### Intermediate 498A

### 6-(3-Chloro-4-methylphenyl)-3-cyclopropyl-7-fluoro-4-oxo-4,5-dihydropyrazolo[1,5-a]pyrazine-2-carboxylic acid

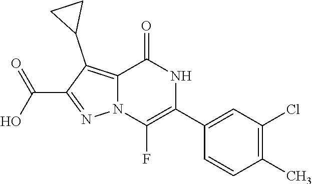 [SMILES: Cc1ccc(-c2[nH]c(=O)c3c(C4CC4)c(C(=O)O)nn3c2F)cc1Cl]

A round bottom flask was charged with 6-(3-chloro-4-methylphenyl)-3-cyclopropyl-4-oxo-4,5-dihydropyrazolo[1,5-a]pyrazine-2-carboxylic acid (Intermediate 318A, 200 mg, 582 μmol) and acetonitrile (25 ml) and 1-chloromethyl-4-fluoro-1,4-diazoniabicyclo[2.2.2]octane bis(tetrafluoroborate) (247 mg, 698 μmol) was added under ice-cooling. The mixture was stirred at RT overnight. More 1-chloromethyl-4-fluoro-1,4-diazoniabicyclo[2.2.2]octane bis(tetrafluoroborate) (247 mg, 698 μmol) was added and stirring was continued overnight. The mixture was concentrated and the residue was taken up in ethyl acetate and washed with water and brine. The organic layer was dried over sodium sulfate and evaporated to dryness. The residue was taken up in pyridine (6.0 ml), before 1-propanephosphonic anhydride (1.0 ml, 50% solution in ethyl acetate, 1.7 mmol) was added the mixture was heated to 90° C. for 3 h. After cooling to RT, the mixture was concentrated. Ethyl acetate and water were added to the residue and the insoluble material was removed by filtration. The layers were separated and the aqueous layer was extracted with ethyl acetate. The combined organic layers were washed with brine, dried over sodium sulfate and evaporated to dryness to afford the title compound (179 mg, 75% of theory, 88% purity) which was used in the next step without further purification.

LC/MS [Method 3]: Rₜ=1.66 min; MS (ESIpos): m/z=362 [M+H]⁺.

### Intermediate 499A

### 4-Chloro-3-(difluoromethyl)benzonitrile

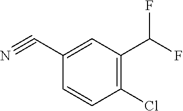 [SMILES: N#Cc1ccc(Cl)c(C(F)F)c1]

N-ethyl-N-(trifluoro-lambda⁴-sulfanyl)ethanamine (DAST, 4.8 ml, 36 mmol) was added dropwise to a solution of 4-chloro-3-formylbenzonitrile (2.00 g, 12.1 mmol) in dichloromethane (40 ml) at 0° C. After complete addition, the reaction mixture was stirred at 0° C. for 1 h and at RT for three days. The reaction mixture was poured into ice-water and extracted with dichloromethane. The combined organic layers were washed with saturated aqueous sodium bicarbonate solution, dried over sodium sulfate and concentrated under reduced pressure to afford the crude title compound which was used in the next step without further purification. Yield: 1.97 g (87% of theory).

¹H-NMR (400 MHz, DMSO-d₆): δ [ppm]=8.22-8.18 (m, 1H), 8.12-8.04 (m, 1H), 7.87 (d, 1H), 7.25 (t, 1H).

### Intermediate 500A

### 1-[4-Chloro-3-(difluoromethyl)phenyl]ethan-1-one

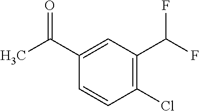

Bromido(methyl)magnesium (21 ml, 1.0 M, 21 mmol) was added to a solution of 4-chloro-3-(difluoromethyl)benzonitrile (Intermediate 499A, 1.97 g, 10.5 mmol) in THF (40 ml) at 0° C. After complete addition, the mixture was allowed to warm to RT and stirred at this temperature for 2 h. The reaction mixture was poured into saturated aqueous ammonium chloride solution, diluted with water and extracted with ethyl acetate. The combined organic layers were dried over sodium sulfate and concentrated under reduced pressure. The residue was purified by column chromatography on silica gel with a cyclohexane/ethyl acetate gradient to give the title compound. Yield: 550 mg (26% of theory).

¹H-NMR (600 MHz, DMSO-d₆): δ [ppm]=8.18 (s, 1H), 8.13 (d, 1H), 7.77 (d, 1H), 7.28 (t, 1H), 2.64 (s, 3H).

### Intermediate 501A

### 2-Bromo-1-[4-chloro-3-(difluoromethyl)phenyl]ethan-1-one

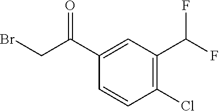 [SMILES: O=C(CBr)c1ccc(Cl)c(C(F)F)c1]

Phenyltrimethylammoniumtribromide (1.01 g, 2.69 mmol) was added in portions to a solution of 1-[4-chloro-3-(difluoromethyl)phenyl]ethan-1-one (Intermediate 500A, 550 mg, 2.69 mmol) in THF (10 ml) and the mixture was stirred at RT for 1 h. The reaction mixture was filtered and the filtrate was concentrated under reduced pressure to afford the crude title compound, which was used in the next step without further purification. Yield: 1.08 g (99% of theory, 70% purity).

¹H-NMR (600 MHz, DMSO-d₆): δ [ppm]=8.24 (d, 1H), 8.19-8.16 (m, 1H), 7.82 (d, 1H), 7.29 (t, 1H), 5.00 (s, 2H).

### Intermediate 502A

### Diethyl 1-{2-[4-chloro-3-(difluoromethyl)phenyl]-2-oxoethyl}-4-cyclopropyl-1H-pyrazole-3,5-dicarboxylate

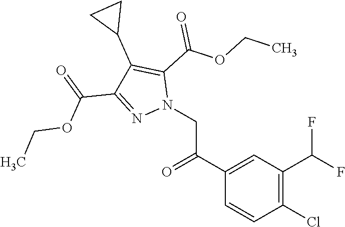 [SMILES: CCOC(=O)c1nn(CC(=O)c2ccc(Cl)c(C(F)F)c2)c(C(=O)OCC)c1C1CC1]

A mixture of diethyl 4-cyclopropyl-1H-pyrazole-3,5-dicarboxylate (Intermediate 187A, 673 mg, 2.67 mmol), 2-bromo-1-[4-chloro-3-(difluoromethyl)phenyl]ethan-1-one (Intermediate 501A, 1.08 g, 70% purity, 2.67 mmol) and potassium carbonate (921 mg, 6.67 mmol) in acetone (23 ml) was stirred at RT overnight. The insoluble material was removed by filtration and the filtrate was concentrated under reduced pressure to afford the crude title compound which was used in the next step without further purification. Yield: 1.40 g (96% of theory, 83% purity).

LC/MS [Method 3]: Rₜ=2.32 min; MS (ESIpos): m/z=455 [M+H]⁺.

### Intermediate 503A

### Ethyl 6-[4-chloro-3-(difluoromethyl)phenyl]-3-cyclopropyl-4-oxo-4,5-dihydropyrazolo[1,5-a]pyrazine-2-carboxylate

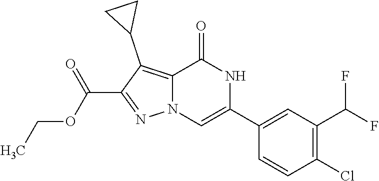 [SMILES: CCOC(=O)c1nn2cc(-c3ccc(Cl)c(C(F)F)c3)[nH]c(=O)c2c1C1CC1]

A mixture of diethyl 1-{2-[4-chloro-3-(difluoromethyl)phenyl]-2-oxoethyl}-4-cyclopropyl-1H-pyrazole-3,5-dicarboxylate (Intermediate 502A, 1.40 g, 83% purity, 2.55 mmol) and ammonium acetate (7.88 g, 102 mmol) in acetic acid (51 ml) was heated to 110° C. and stirred overnight at this temperature. After cooling to RT, the reaction mixture was poured into water. The precipitate was collected by filtration, washed with water and dried to afford the title compound. Yield: 1.10 g (95% of theory, 90% purity).

LC/MS [Method 11]: Rₜ=3.50 min; MS (ESIpos): m/z=408 [M+H]⁺.

### Intermediate 504A

### 6-[4-Chloro-3-(difluoromethyl)phenyl]-3-cyclopropyl-4-oxo-4,5-dihydropyrazolo[1 ,5-a]pyrazine-2-carboxylic acid

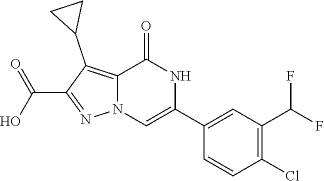 [SMILES: O=C(O)c1nn2cc(-c3ccc(Cl)c(C(F)F)c3)[nH]c(=O)c2c1C1CC1]

A mixture of ethyl 6-[4-chloro-3-(difluoromethyl)phenyl]-3-cyclopropyl-4-oxo-4,5-dihydropyrazolo[1,5-a]pyrazine-2-carboxylate (Intermediate 503A, 1.10 g, 90% purity, 2.43 mmol) and lithium hydroxide ((581 mg, 24.3 mmol) in methanol (20 ml) and water (10 ml) was stirred at RT overnight. The methanol was removed under reduced pressure and the remaining solution was acidified with 1.0 M hydrochloric acid. The precipitate was collected by filtration and dried to afford the title compound. Yield: 770 mg (84% of theory).

LC/MS [Method 3]: Rₜ=1.58 min; MS (ESIpos): m/z=380 [M+H]⁺.

¹H-NMR (600 MHz, DMSO-d₆): 6 [ppm]=13.03 (br s, 1H), 11.62 (s, 1H), 8.13 (s, 1H), 8.06 (d, 1H), 7.92 (dd, 1H), 7.72 (d, 1H), 7.27 (t, 1H), 2.76-2.65 (m, 1H), 1.27-1.22 (m, 2H), 0.97-0.90 (m, 2H).

### Intermediate 505A

### Diethyl 1-[2-(4-chlorophenyl)-2-oxoethyl]-4-(propan-2-yl)-1H-pyrazole-3,5-dicarboxylate

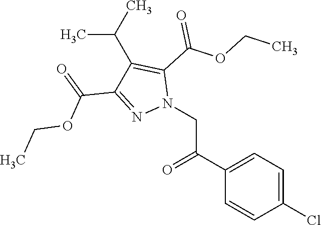 [SMILES: CCOC(=O)c1nn(CC(=O)c2ccc(Cl)cc2)c(C(=O)OCC)c1C(C)C]

A mixture of diethyl 4-(propan-2-yl)-1H-pyrazole-3,5-dicarboxylate (Intermediate 16A, 740 mg, 97% purity, 2.82 mmol), 2-bromo-1-(4-chlorophenyl)ethan-1-one (725 mg, 3.11 mmol) and potassium carbonate (975 mg, 7.06 mmol) in acetone (25 ml) was stirred at RT overnight. The insoluble material was removed by filtration. The filtrate was concentrated under reduced pressure to afford the crude title compound which was used in the next step without further purification. Yield: 1.27 g (quant., 90% purity).

LC/MS [Method 3]: Rₜ =Rₜ=2.37 min; MS (ESIpos): m/z=407 [M+H]⁺.

### Intermediate 506A

### Ethyl 6-(4-chlorophenyl)-4-oxo-3-(propan-2-yl)-4,5-dihydropyrazolo[1,5-a]pyrazine-2-carboxylate

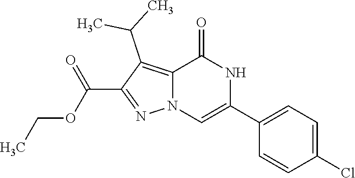 [SMILES: CCOC(=O)c1nn2cc(-c3ccc(Cl)cc3)[nH]c(=O)c2c1C(C)C]

A mixture of diethyl 1-[2-(4-chlorophenyl)-2-oxoethyl]-4-(propan-2-yl)-1H-pyrazole-3,5-dicarboxylate (Intermediate 505A, 1.27 g, 90% purity, 2.82 mmol) and ammonium acetate (8.70 g, 113 mmol) in acetic acid (60 ml) was heated to 110° C. overnight. After cooling to RT, the mixture was poured into water and the precipitate was collected by filtration, washed with water and MTBE and dried to afford the title compound. Yield: 787 mg (78% of theory, 100% purity).

LC/MS [Method 3]: Rₜ=2.12 min; MS (ESIpos): m/z=360 [M+H]⁺.

### Intermediate 507A

### 6-(4-Chlorophenyl)-4-oxo-3-(propan-2-yl)-4,5-dihydropyrazolo[1,5-a]pyrazine-2-carboxylic acid

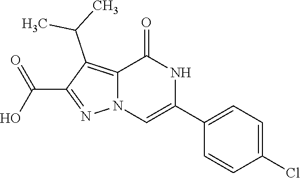 [SMILES: CC(C)c1c(C(=O)O)nn2cc(-c3ccc(Cl)cc3)[nH]c(=O)c12]

A mixture of ethyl 6-(4-chlorophenyl)-4-oxo-3-(propan-2-yl)-4,5-dihydropyrazolo[1,5-a]pyrazine-2-carboxylate (Intermediate 506A, 787 mg, 2.19 mmol) and lithium hydroxide (524 mg, 21.9 mmol) in methanol (20 ml) and water (10 ml) was stirred at RT overnight. The methanol was removed under reduced pressure and the remaining solution was acidified with 1.0 M hydrochloric acid. The precipitate was collected by filtration and dried to afford the title compound.

Yield: 600 mg (83% of theory).

LC/MS [Method 3]: Rₜ=1.58 min; MS (ESIneg): m/z=330 [M−H]⁻.

¹H-NMR (600 MHz, DMSO-d₆): δ [ppm]=13.09 (br s, 1H), 11.56 (s, 1H), 8.07-8.05 (m, 1H), 7.79-7.76 (m, 2H), 7.58-7.54 (m, 2H), 4.13 (spt, 1H), 1.37 (d, 6H).

### Intermediate 508A

### Diethyl 1-[2-(4-chlorophenyl)-2-oxoethyl]-4-cyclopropyl-1H-pyrazole-3,5-dicarboxylate

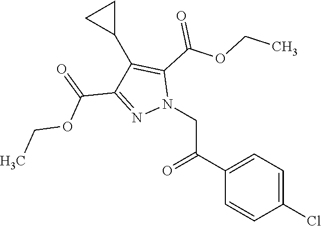 [SMILES: CCOC(=O)c1nn(CC(=O)c2ccc(Cl)cc2)c(C(=O)OCC)c1C1CC1]

A mixture of diethyl 4-cyclopropyl-1H-pyrazole-3,5-dicarboxylate (Intermediate 187A, 540 mg, 2.14 mmol), 2-bromo-1-(4-chlorophenyl)ethan-1-one (550 mg, 2.36 mmol) and potassium carbonate (740 mg, 5.35 mmol) in acetone (20 ml) was stirred at RT overnight. The insoluble material was removed by filtration. The filtrate was concentrated under reduced pressure to afford the crude title compound which was used in the next step without further purification. Yield: 940 mg (98% of theory, 90% purity).

LC/MS [Method 3]: Rₜ=2.27 min; MS (ESIpos): m/z=405 [M+H]⁺.

### Intermediate 509A

### Ethyl 6-(4-chlorophenyl)-3-cyclopropyl-4-oxo-4,5-dihydropyrazolo[1,5-a]pyrazine-2-carboxylate

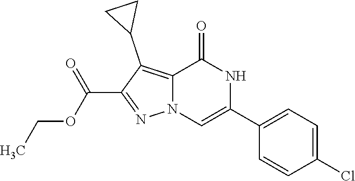 [SMILES: CCOC(=O)c1nn2cc(-c3ccc(Cl)cc3)[nH]c(=O)c2c1C1CC1]

A mixture of diethyl 1-[2-(4-chlorophenyl)-2-oxoethyl]-4-cyclopropyl-1H-pyrazole-3,5-dicarboxylate (Intermediate 508A, 940 mg, 90% purity, 2.09 mmol) and ammonium acetate (6.44 g, 83.6 mmol) in acetic acid (40 ml) was heated to 110° C. overnight. After cooling to RT, the mixture was poured into water and the precipitate was collected by filtration, washed with water and MTBE and dried to afford the title compound. Yield: 450 mg (60% of theory).

LC/MS [Method 3]: Rₜ=2.00 min; MS (ESIpos): m/z=358 [M+H]⁺.

### Intermediate 510A

### 6-(4-Chlorophenyl)-3-cyclopropyl-4-oxo-4,5-dihydropyrazolo[1,5-a]pyrazine-2-carboxylic acid

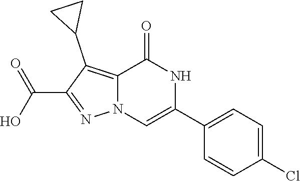 [SMILES: O=C(O)c1nn2cc(-c3ccc(Cl)cc3)[nH]c(=O)c2c1C1CC1]

A mixture of ethyl 6-(4-chlorophenyl)-3-cyclopropyl-4-oxo-4,5-dihydropyrazolo[1,5-a]pyrazine-2-carboxylate (Intermediate 509A, 450 mg, 1.26 mmol) and lithium hydroxide (301 mg, 12.6 mmol) in methanol (10 ml) and water (5.0 ml) was stirred at RT overnight. The methanol was removed under reduced pressure and the remaining solution was acidified with 1.0 M hydrochloric acid. The precipitate was collected by filtration and dried to afford the title compound. Yield: 385 mg (93% of theory).

LC/MS [Method 3]: Rₜ=1.48 min; MS (ESIneg): m/z=328 [M−H]⁻.

¹H-NMR (600 MHz, DMSO-d₆): δ [ppm]=13.04 (br s, 1H), 11.49 (s, 1H), 8.05-8.03 (m, 1H), 7.78-7.75 (m, 2H), 7.58-7.53 (m, 2H), 2.75-2.70 (m, 1H), 1.26-1.23 (m, 2H), 0.94-0.90 (m, 2H).

### Intermediate 511A

### Diethyl 1-[2-(3-chlorophenyl)-2-oxoethyl]-4-cyclopropyl-1H-pyrazole-3,5-dicarboxylate

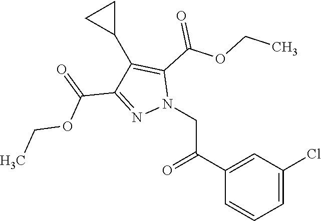 [SMILES: CCOC(=O)c1nn(CC(=O)c2cccc(Cl)c2)c(C(=O)OCC)c1C1CC1]

A mixture of diethyl 4-cyclopropyl-1H-pyrazole-3,5-dicarboxylate (Intermediate 187A, 500 mg, 1.98 mmol), 2-bromo-1-(3-chlorophenyl)ethan-1-one (555 mg, 2.38 mmol) and potassium carbonate (685 mg, 4.95 mmol) in acetone (15 ml) was stirred at RT overnight. The insoluble material was removed by filtration. The filtrate was concentrated under reduced pressure to afford the crude title compound which was used in the next step without further purification. Yield: 950 mg (99% of theory, 84% purity).

LC/MS [Method 3]: Rₜ=2.29 min; MS (ESIneg): m/z=403 [M−H]⁻.

### Intermediate 512A

### Ethyl 6-(3-chlorophenyl)-3-cyclopropyl-4-oxo-4,5-dihydropyrazolo[1,5-a]pyrazine-2-carboxylate

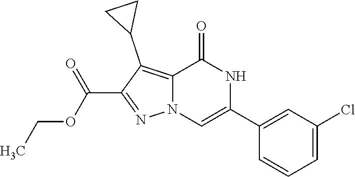 [SMILES: CCOC(=O)c1nn2cc(-c3cccc(Cl)c3)[nH]c(=O)c2c1C1CC1]

A mixture of diethyl 1-[2-(3-chlorophenyl)-2-oxoethyl]-4-cyclopropyl-1H-pyrazole-3,5-dicarboxylate (Intermediate 511A, 950 mg, 2.35 mmol) and ammonium acetate (7.23 g, 93.9 mmol) in acetic acid (26 ml) was heated to 110° C. overnight. After cooling to RT, the mixture was poured into water and the precipitate was collected by filtration, washed with water and MTBE and dried to afford the title compound. Yield: 691 mg (70% of theory, 85% purity).

LC/MS [Method 7]: Rₜ=1.00 min; MS (ESIpos): m/z=358 [M+H]⁺.

### Intermediate 513A

### 6-(3-Chlorophenyl)-3-cyclopropyl-4-oxo-4,5-dihydropyrazolo[1,5-a]pyrazine-2-carboxylic acid

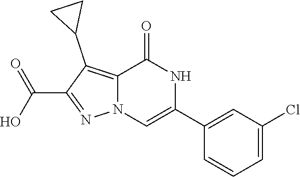 [SMILES: O=C(O)c1nn2cc(-c3cccc(Cl)c3)[nH]c(=O)c2c1C1CC1]

A mixture of ethyl ethyl 6-(3-chlorophenyl)-3-cyclopropyl-4-oxo-4,5-dihydropyrazolo[1,5-a]-pyrazine-2-carboxylate (Intermediate 512A, 690 mg, 1.93 mmol) and lithium hydroxide (231 mg, 9.64 mmol) in methanol (7.5 ml) and water (3.8 ml) was stirred at RT overnight. The methanol was removed under reduced pressure and the remaining solution was acidified with 1.0 M hydrochloric acid. The precipitate was collected by filtration, washed with MTBE and dried to afford the title compound. Yield: 376 mg (54% of theory, 91% purity).

LC/MS [Method 3]: Rₜ=1.48 min; MS (ESIpos): m/z=330 [M+H]⁺.

### Intermediate 514A

### Diethyl 1-[2-(3-chlorophenyl)-2-oxoethyl]-4-(propan-2-yl)-1H-pyrazole-3,5-dicarboxylate

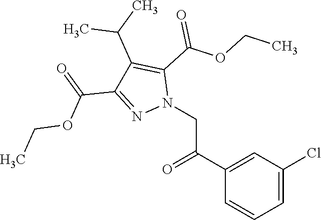 [SMILES: CCOC(=O)c1nn(CC(=O)c2cccc(Cl)c2)c(C(=O)OCC)c1C(C)C]

A mixture of diethyl 4-(propan-2-yl)-1H-pyrazole-3,5-dicarboxylate (Intermediate 16A, (500 mg, 1.97 mmol), 2-bromo-1-(3-chlorophenyl)ethan-1-one (551 mg, 2.36 mmol) and potassium carbonate (679 mg, 4.92 mmol) in acetone (15 ml) was stirred at RT overnight. The insoluble material was removed by filtration. The filtrate was concentrated under reduced pressure to afford the crude title compound which was used in the next step without further purification. Yield: 900 mg (99% of theory, 88% purity).

LC/MS [Method 3]: Rₜ=2.40 min; MS (ESIpos): m/z=407 [M+H]⁺.

### Intermediate 515A

### Ethyl 6-(3-chlorophenyl)-4-oxo-3-(propan-2-yl)-4,5-dihydropyrazolo[1,5-a]pyrazine-2-carboxylate

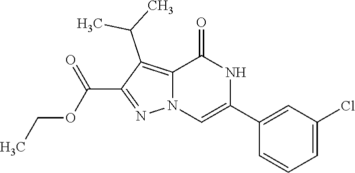 [SMILES: CCOC(=O)c1nn2cc(-c3cccc(Cl)c3)[nH]c(=O)c2c1C(C)C]

A mixture of diethyl 1-[2-(3-chlorophenyl)-2-oxoethyl]-4-(propan-2-yl)-1H-pyrazole-3,5-dicarboxylate (Intermediate 514A, 950 mg, 2.33 mmol) and ammonium acetate (7.20 g, 93.4 mmol) in acetic acid (26 ml) was heated to 110° C. overnight. After cooling to RT, the mixture was poured into water and the precipitate was collected by filtration, washed with water and MTBE and dried to afford the title compound. Yield: 710 mg (85% of theory).

LC/MS [Method 7]: Rₜ=1.10 min; MS (ESIpos): m/z=360 [M+H]⁺.

### Intermediate 516A

### 6-(3-Chlorophenyl)-4-oxo-3-(propan-2-yl)-4,5-dihydropyrazolo[1,5-a]pyrazine-2-carboxylic acid

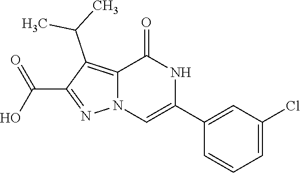 [SMILES: CC(C)c1c(C(=O)O)nn2cc(-c3cccc(Cl)c3)[nH]c(=O)c12]

A mixture of ethyl 6-(3-chlorophenyl)-4-oxo-3-(propan-2-yl)-4,5-dihydropyrazolo[1,5-a]pyrazine-2-carboxylate (Intermediate 509A, 710 mg, 1.97 mmol) and lithium hydroxide (236 mg, 9.87 mmol) in methanol (8.0 ml) and water (4.0 ml) was stirred at RT overnight. The methanol was removed under reduced pressure and the remaining solution was acidified with 1.0 M hydrochloric acid. The precipitate was collected by filtration and dried to afford the title compound.

Yield: 598 mg (91% of theory).

LC/MS [Method 3]: Rₜ=1.58 min; MS (ESIpos): m/z=332 [M+H]⁺.

### EXAMPLE COMPOUNDS

General Procedure 1-A: Amide Coupling using HATU and DIPEA at 50° C.

N,N-Diisopropylethylamine (DIPEA, 3.0 eq) and HATU (1.2 eq) were added to a solution/suspension of the corresponding carboxylic acid (1.0 eq) in N-methylpyrrolidone (NMP, 5-20 ml per mmol carboxylic acid) or N,N-dimethylformamide (DMF, 5-20 ml per mmol carboxylic acid) or 1,2-dichloroethane (10-20 ml per mmol carboxylic acid). The mixture was stirred at 50° C. After 1 h, the corresponding amine (1.1-2.0 eq) was added, and stirring was continued at 50° C. overnight. After cooling to RT, the reaction mixture was treated as described in the individual examples.

General Procedure 1-B: Amide Coupling using CDI and DMAP

DMAP (0.5 eq) and CDI (1.0-2.0 eq) were added to a solution/suspension of the corresponding carboxylic acid (1.0 eq) in N-methylpyrrolidone (NMP, 5-20 ml per mmol carboxylic acid). The mixture was stirred at 50° C. After 1 h, the corresponding amine (1.0-1.1 eq) was added, and stirring was continued at 50° C. overnight. After cooling to RT, the reaction mixture was treated as described in the individual examples.

General Procedure 1-C: Amide Coupling using HATU and DIPEA at RT

N,N-Diisopropylethylamine (DIPEA, 2.0-3.0 eq) and HATU (1.1-1.3 eq) were added to a solution/suspension of the corresponding carboxylic acid (1.0 eq) in N-methylpyrrolidone (NMP, 5-20 ml per mmol carboxylic acid) or N,N-dimethylformamide (DMF, 5-20 ml per mmol carboxylic acid) or 1,2-dichloroethane (10-20 ml per mmol carboxylic acid). The corresponding amine (1.0-1.2 eq) was added, and the mixture was stirred at RT overnight. After this, the reaction mixture was treated as described in the individual examples.

### Example 1-1

### 6-(Naphthalen-2-yl)-4-oxo-N-{(1R)-1-[4-(trifluoromethyl)phenyl]ethyl}-4,5-dihydropyrazolo[1,5-a]-pyrazine-2-carboxamide

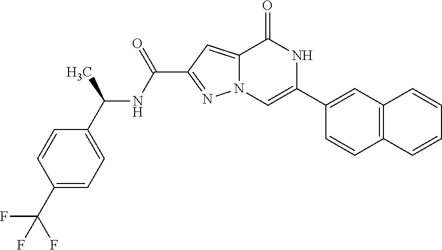 [SMILES: C[C@@H](NC(=O)c1cc2c(=O)[nH]c(-c3ccc4ccccc4c3)cn2n1)c1ccc(C(F)(F)F)cc1]

The title compound was prepared according to general procedure 1-A starting from 6-(naphthalen-2-yl)-4-oxo-4,5-dihydropyrazolo[1,5-a]pyrazine-2-carboxylic acid (Intermediate 3A, 100 mg, 328 μmol), (1R)-1-[4-(trifluoromethyl)phenyl]ethanamine (74.4 mg, 393 μmol), HATU (149 mg, 393 μmol) and N,N-diisopropylethylamine (170 μl, 980 μmol) in 1,2-dichloroethane (5.0 ml). The reaction mixture was poured into water and extracted with dichloromethane. The combined organic layers were dried over magnesium sulfate, and the solvent was evaporated under reduced pressure. The residue was purified by silica gel column chromatography (eluent: dichloromethane/methanol 20:1). Yield: 98.7 mg (62% of theory).

LC/MS [Method 3]: Rₜ=2.08 min; MS (ESIpos): m/z=477 [M+H]⁺.

¹H-NMR (400 MHz, DMSO-d₆): δ [ppm]=11.87 (br. s, 1H), 9.00 (d, 1H), 8.38 (s, 1H), 8.18 (s, 1H), 8.06-7.96 (m, 3H), 7.88 (dd, 1H), 7.73-7.68 (m, 2H), 7.68-7.63 (m, 2H), 7.63-7.58 (m, 2H), 7.43 (s, 1H), 5.32-5.18 (m, 1H), 1.54 (d, 3H).

### Example 1-2

### N-[(1R)-2-Methoxy-1-phenylethyl]-6-(naphthalen-2-yl)-4-oxo-4,5-dihydropyrazolo[1,5-a]pyrazine-2-carboxamide

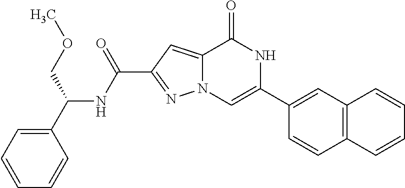 [SMILES: COC[C@H](NC(=O)c1cc2c(=O)[nH]c(-c3ccc4ccccc4c3)cn2n1)c1ccccc1]

The title compound was prepared according to general procedure 1-B starting from 6-(naphthalen-2-yl)-4-oxo-4,5-dihydropyrazolo[1,5-a]pyrazine-2-carboxylic acid (Intermediate 3A, 100 mg, 328 μmol), (1R)-2-methoxy-1-phenylethanamine (49.5 mg, 328 μmol), CDI (106 mg, 655 μmol) and DMAP (20.0 mg, 164 μmol) in N-methylpyrrolidone (5.0 ml, 52 mmol). The reaction mixture was directly purified by preparative HPLC (Method P1). Yield: 89.9 mg (63% of theory).

LC/MS [Method 3]: Rₜ=1.90 min; MS (ESIpos): m/z=439 [M+H]⁺.

¹H-NMR (400 MHz, DMSO-d₆): δ [ppm]=11.88 (br. s, 1H), 8.76 (d, 1H), 8.39 (s, 1H), 8.23 (s, 1H), 8.04 (d, 1H), 8.02-7.96 (m, 2H), 7.89 (dd, 1H), 7.64-7.57 (m, 2H), 7.47-7.39 (m, 3H), 7.39-7.30 (m, 2H), 7.30-7.24 (m, 1H), 5.31-5.24 (m, 1H), 3.78 (dd, 1H), 3.61 (dd, 1H), 3.31 (s, 3H).

### Example 1-3

### N-[(1S)-2-Hydroxy-2-methyl-1-phenylpropyl]-6-(naphthalen-2-yl)-4-oxo-4,5-dihydropyrazolo-[1,5-a]pyrazine-2-carboxamide

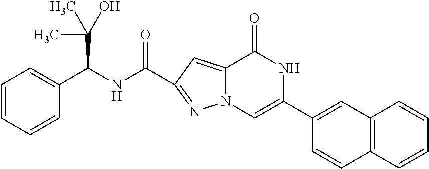 [SMILES: CC(C)(O)[C@@H](NC(=O)c1cc2c(=O)[nH]c(-c3ccc4ccccc4c3)cn2n1)c1ccccc1]

The title compound was prepared according to general procedure 1-B starting from 6-(naphthalen-2-yl)-4-oxo-4,5-dihydropyrazolo[1,5-a]pyrazine-2-carboxylic acid (Intermediate 3A, 100 mg, 328 μmol), (1S)-1-amino-2-methyl-1-phenylpropan-2-ol (Intermediate 4A, 59.5 mg, 360 μmol), CDI (58.4 mg, 360 μmol) and DMAP (20.0 mg, 164 μmol) in NMP (2.3 ml). The reaction mixture was directly purified by preparative HPLC (Method P2). Yield: 43.0 mg (28% of theory).

LC/MS [Method 3]: Rₜ=1.83 min; MS (ESIneg): m/z=451 [M−H]⁻.

¹H-NMR (400 MHz, DMSO-d₆): δ [ppm]=11.88 (s, 1H), 8.41 (s, 1H), 8.38 (s, 1H), 8.31 (d, 1H), 8.04 (d, 1H), 8.01-7.95 (m, 2H), 7.91 (dd, 1H), 7.63-7.58 (m, 2H), 7.44-7.39 (m, 2H), 7.34-7.27 (m, 3H), 7.27-7.22 (m, 1H), 4.97 (s, 1H), 4.86 (d, 1H), 1.28 (s, 3H), 0.99 (s, 3H).

### Example 1-4

### rac-N-[2-(Dimethylamino)-1-phenylethyl]-6-(naphthalen-2-yl)-4-oxo-4,5-dihydropyrazolo[1,5-a]-pyrazine-2-carboxamide

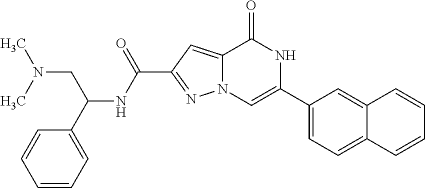 [SMILES: CN(C)CC(NC(=O)c1cc2c(=O)[nH]c(-c3ccc4ccccc4c3)cn2n1)c1ccccc1]

To a solution of 6-(naphthalen-2-yl)-4-oxo-4,5-dihydropyrazolo[1,5-a]pyrazine-2-carboxylic acid (Intermediate 3A, 1.50 g, 4.91 mmol) in DMF (50.0 ml) was added DIPEA (2.6 ml, 15 mmol) followed by 1-(3-dimethylaminopropyl)-3-ethylcarbodiimide hydrochloride (1.04 g, 5.40 mmol) and 1-hydroxy-7-azabenzotriazole (334 mg, 2.46 mmol). The mixture was stirred at RT for 1 h. N-(2-Amino-2-phenylethyl)-N,N-dimethylamine (888 mg, 5.40 mmol) was added, and the mixture was stirred at 50° C. overnight. After cooling to RT, the reaction mixture was poured into water, and the precipitate was filtered off and washed with water and ethyl acetate. The solid was suspended in water and treated with diluted hydrochloric acid until a clear solution was obtained. The aqueous phase was washed with ethyl acetate three times and the organic washings were discarded. The residual ethyl acetate was removed from the aqueous layer by distillation, and the aqueous phase was treated with DIPEA until basic pH was reached. The precipitate was filtered off, washed with water and dried at 50° C. The obtained solid was taken up in DMSO and water until most of the material had dissolved, and the mixture was lyophilized to afford the title compound. Yield: 1.21 g (55% of theory).

LC/MS [Method 3]: Rₜ=1.20 min; MS (ESIpos): m/z=452 [M+H]⁺.

¹H-NMR (400 MHz, DMSO-d₆): δ [ppm]=11.86 (br. s, 1H), 8.62 (d, 1H), 8.39 (s, 1H), 8.23 (s, 1H), 8.04 (d, 1H), 8.02-7.96 (m, 2H), 7.93-7.87 (m, 1H), 7.64-7.57 (m, 2H), 7.43 (d, 2H), 7.39 (s, 1H), 7.37-7.31 (m, 2H), 7.28-7.22 (m, 1H), 5.18-5.10 (m, 1H), 2.90-2.80 (m, 1H), 2.47-2.38 (m, 1H), 2.22 (s, 6H).

### Example 1-5

### tert.-Butyl 3-ethyl-3-({[6-(naphthalen-2-yl)-4-oxo-4,5-dihydropyrazolo[1,5-a]pyrazin-2-yl]carbonyl}-amino)azetidine-1-carboxylate

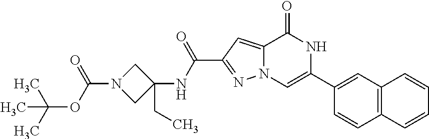 [SMILES: CCC1(NC(=O)c2cc3c(=O)[nH]c(-c4ccc5ccccc5c4)cn3n2)CN(C(=O)OC(C)(C)C)C1]

The title compound was prepared according to general procedure 1-B starting from 6-(naphthalen-2-yl)-4-oxo-4,5-dihydropyrazolo[1,5-a]pyrazine-2-carboxylic acid (Intermediate 3A, 100 mg, 328 μmol), tert.-butyl 3-amino-3-ethylazetidine-1-carboxylate (Intermediate 8A, 65.6 mg, 328 μmol), CDI (106 mg, 655 μmol) and DMAP (20.0 mg, 164 μmol) in N-methylpyrrolidone (5.0 ml). After cooling to RT, the reaction mixture was directly purified by preparative HPLC (Method P1). Yield: 58.2 mg (33% of theory, 91% purity).

LC/MS [Method 3]: Rₜ=2.01 min; MS (ESIneg): m/z=486 [M−H]⁻.

¹H-NMR (400 MHz, DMSO-d₆): δ [ppm]=11.88 (s, 1H), 8.87 (s, 1H), 8.38 (s, 1H), 8.15 (s, 1H), 8.04 (d, 1H), 8.02-7.96 (m, 2H), 7.93-7.82 (m, 1H), 7.64-7.57 (m, 2H), 7.40 (s, 1H), 4.11-3.95 (m, 2H), 3.84-3.71 (m, 2H), 1.94 (q, 2H), 1.39 (s, 9H), 0.86 (t, 3H).

### Example 1-6

### tert.-Butyl 3-(6-methoxypyridin-3-yl)-3-({[6-(naphthalen-2-yl)-4-oxo-4,5-dihydropyrazolo[1,5-a]-pyrazin-2-yl]carbonyl}amino)azetidine-1-carboxylate

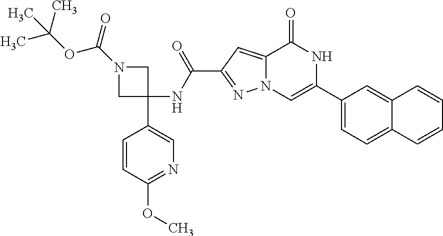 [SMILES: COc1ccc(C2(NC(=O)c3cc4c(=O)[nH]c(-c5ccc6ccccc6c5)cn4n3)CN(C(=O)OC(C)(C)C)C2)cn1]

DIPEA (350 μl, 2.0 mmol), 1-(3-dimethylaminopropyl)-3-ethylcarbodiimide hydrochloride (140 mg, 732 μmol) and 1-hydroxy-7-azabenzotriazole (550 μl, 0.60 M in DMF, 330 μmol) were added to a mixture of 6-(naphthalen-2-yl)-4-oxo-4,5-dihydropyrazolo[1,5-a]pyrazine-2-carboxylic acid (Intermediate 3A, 203 mg, 666 μmol) and DMF (11.6 ml) at RT. After stirring at RT for 1.5 h, a solution of tert.-butyl 3-amino-3-(6-methoxypyridin-3-yl)azetidine-1-carboxylate (Intermediate 11A, 220 mg, 93% purity, 732 μmol) in DMF (2.0 ml) was added, and stirring at RT was continued overnight. The mixture was diluted with water and extracted with ethyl acetate. The organic layers were combined and upon standing, a solid precipitated. The precipitate was filtered off and dried to afford the title compound. Yield: 192 mg (51% of theory).

LC/MS [Method 3]: Rₜ=1.94 min; MS (ESIpos): m/z=567 [M+H]⁺.

¹H-NMR (400 MHz, DMSO-d₆): δ [ppm]=11.90 (s, 1H), 9.63 (s, 1H), 8.38 (s, 1H), 8.26 (d, 1H), 8.15 (s, 1H), 8.05 (d, 1H), 8.02-7.96 (m, 2H), 7.89-7.86 (m, 1H), 7.83-7.80 (m, 1H), 7.64-7.58 (m, 2H), 7.40 (s, 1H), 6.85 (d, 1H), 4.38 (br. d, 2H), 4.30-4.14 (m, 2H), 3.84 (s, 3H), 1.41 (s, 9H).

### Example 1-7

### N-[(1S)-2-(Morpholin-4-yl)-1-phenylethyl]-6-(naphthalen-2-yl)-4-oxo-4,5-dihydropyrazolo[1,5-a]-pyrazine-2-carboxamide formic acid salt

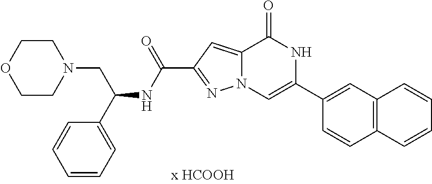 [SMILES: O=C(N[C@H](CN1CCOCC1)c1ccccc1)c1cc2c(=O)[nH]c(-c3ccc4ccccc4c3)cn2n1]

CDI (83.9 mg, 517 μmol) and DMAP (15.8 mg, 129 μmol) were added to a solution of 6-(naphthalen-2-yl)-4-oxo-4,5-dihydropyrazolo[1,5-a]pyrazine-2-carboxylic acid (Intermediate 3A, 78.9 mg, 259 μmol) in DMF (2.11 ml), and the mixture was stirred at 50° C. for 1 h. In a separate flask, (1S)-2-(morpholin-4-yl)-1-phenylethanamine dihydrochloride (Intermediate 14A, 95.0 mg, 76% purity, 259 μmol) was dissolved in DMF (2.11 ml), and triethylamine (110 μl, 780 μmol) was added. This mixture was stirred for 1 h at RT before it was added to the previously prepared solution of the carboxylic acid. The resulting mixture was stirred at 50° C. overnight. After cooling to RT, water was added, and the mixture was extracted with ethyl acetate. The combined organic layers were washed with brine and dried over magnesium sulfate, and the solvent was evaporated under reduced pressure. The residue was taken up in acetonitrile (2.00 ml), and the solid was filtered off and washed with an additional amount of acetonitrile (0.50 ml). The obtained filter cake was dissolved in DMSO and purified by RP-HPLC to afford the title compound. Yield: 8.60 mg (6% of theory).

LC/MS [Method 7]: Rₜ=0.72 min; MS (ESIpos): m/z=494 [M+H]⁺.

¹H-NMR (400 MHz, DMSO-d₆): δ [ppm]=11.88 (br. s, 1H), 8.72 (d, 1H), 8.39 (s, 1H), 8.22 (s, 1H), 8.04 (d, 1H), 8.02-7.97 (m, 2H), 7.92-7.88 (m, 1H), 7.63-7.58 (m, 2H), 7.44 (d, 2H), 7.40 (s, 1H), 7.38-7.31 (m, 2H), 7.27-7.22 (m, 1H), 5.26-5.20 (m, 1H), 3.60-3.51 (m, 4H), 2.96-2.85 (m, 1H), 2.47-2.38 (m, 2H).

### Example 1-8

### N-[(1R)-1-(4-Fluorophenyl)ethyl]-6-(naphthalen-2-yl)-4-oxo-3-(propan-2-yl)-4,5-dihydropyrazolo-[1,5-a]pyrazine-2-carboxamide

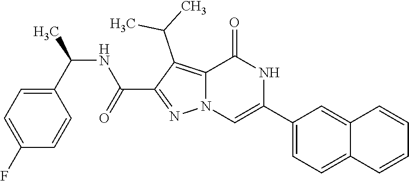 [SMILES: CC(C)c1c(C(=O)N[C@H](C)c2ccc(F)cc2)nn2cc(-c3ccc4ccccc4c3)[nH]c(=O)c12]

6-(Naphthalen-2-yl)-4-oxo-3-(propan-2-yl)-4,5-dihydropyrazolo[1,5-a]pyrazine-2-carboxylic acid (Intermediate 19A, 200 mg, 576 μmol) was dissolved in DMF (14.0 ml), and (1R)-1-(4-fluorophenyl)ethanamine (80.1 mg, 576 μmol), N,N-diisopropylethylamine (300 μl, 1.7 mmol) and HATU (241 mg, 633 μmol) were added. The mixture was stirred at RT overnight. The reaction mixture was diluted with water and dichloromethane, the layers were separated, and the aqueous layer was extracted with dichloromethane. The combined organic layers were washed with brine, dried over sodium sulfate and evaporated. The residue was purified by preparative HPLC (Method P12) to afford the title compound. Yield: 141 mg (52% of theory).

LC/MS [Method 33]: Rₜ=1.44 min; MS (ESIpos): m/z=469 [M+H]⁺.

¹H-NMR (400 MHz, DMSO-d₆): δ [ppm]=11.66 (s, 1H), 8.76 (d, 1H), 8.37 (s, 1H), 8.13 (s, 1H), 8.02 (d, 1H), 8.00-7.94 (m, 2H), 7.92-7.82 (m, 1H), 7.63-7.55 (m, 2H), 7.51-7.42 (m, 2H), 7.20-7.13 (m, 2H), 5.21-5.13 (m, 1H), 4.12-4.02 (m, 1H), 1.48 (d, 3H), 1.34 (d, 3H), 1.32 (d, 3H).

### Example 1-9

### N-[(1S)-1-(4-Fluorophenyl)ethyl]-6-(naphthalen-2-yl)-4-oxo-3-(propan-2-yl)-4,5-dihydropyrazolo-[1,5-a]pyrazine-2-carboxamide

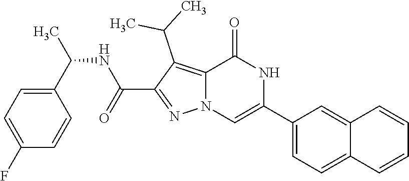 [SMILES: CC(C)c1c(C(=O)N[C@@H](C)c2ccc(F)cc2)nn2cc(-c3ccc4ccccc4c3)[nH]c(=O)c12]

6-(Naphthalen-2-yl)-4-oxo-3-(propan-2-yl)-4,5-dihydropyrazolo[1,5-a]pyrazine-2-carboxylic acid (Intermediate 19A, 200 mg, 576 μmol) was dissolved in DMF (14.0 ml), and (1S)-1-(4-fluoro-phenyl)ethanamine (80.1 mg, 576 μmol), N,N-diisopropylethylamine (300 μl, 1.7 mmol) and HATU (241 mg, 633 μmol) were added. The mixture was stirred at RT overnight. The reaction mixture was diluted with water and dichloromethane, the layers were separated, and the aqueous layer was extracted with dichloromethane. The combined organic layers were washed with brine, dried over sodium sulfate and evaporated. The residue was purified by preparative HPLC (Method P12) to afford the title compound. Yield: 144 mg (53% of theory).

LC/MS [Method 33]: Rₜ=1.45 min; MS (ESIpos): m/z=469 [M+H]⁺.

¹H-NMR (400 MHz, DMSO-d₆): δ [ppm]=11.66 (s, 1H), 8.76 (d, 1H), 8.37 (s, 1H), 8.13 (s, 1H), 8.02 (d, 1H), 8.00-7.94 (m, 2H), 7.92-7.82 (m, 1H), 7.63-7.55 (m, 2H), 7.51-7.42 (m, 2H), 7.20-7.13 (m, 2H), 5.21-5.13 (m, 1H), 4.12-4.02 (m, 1H), 1.48 (d, 3H), 1.40-1.26 (m, 6H).

### Example 1-10

### N-(3,4-Dimethoxybenzyl)-6-(naphthalen-2-yl)-4-oxo-3-(propan-2-yl)-4,5-dihydropyrazolo[1,5-a]-pyrazine-2-carboxamide

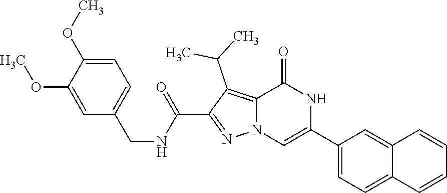 [SMILES: COc1ccc(CNC(=O)c2nn3cc(-c4ccc5ccccc5c4)[nH]c(=O)c3c2C(C)C)cc1OC]

6-(Naphthalen-2-yl)-4-oxo-3-(propan-2-yl)-4,5-dihydropyrazolo[1,5-a]pyrazine-2-carboxylic acid (Intermediate 19A, 200 mg, 576 μmol) was dissolved in DMF (14.0 ml), and 1-(3,4-dimethoxyphenyl)methanamine (96.3 mg, 576 μmol), N,N-diisopropylethylamine (300 μl, 1.7 mmol) and HATU (241 mg, 633 μmol) were added. The mixture was stirred at RT overnight. The reaction mixture was diluted with water and dichloromethane, the layers were separated, and the aqueous layer was extracted with dichloromethane. The combined organic layers were washed with brine, dried over sodium sulfate and evaporated. The residue was purified by preparative HPLC (Method P12) to afford the title compound. Yield: 153 mg (54% of theory).

LC/MS [Method 34]: Rₜ=1.34 min; MS (ESIpos): m/z=497 [M+H]⁺.

¹H-NMR (400 MHz, DMSO-d₆): δ [ppm]=11.66 (br. s, 1H), 8.81-8.72 (m, 1H), 8.36 (s, 1H), 8.07 (s, 1H), 8.01 (d, 1H), 7.99-7.93 (m, 2H), 7.91-7.80 (m, 1H), 7.63-7.54 (m, 2H), 7.02-6.95 (m, 1H), 6.93-6.85 (m, 2H), 4.41 (d, 2H), 4.21-4.09 (m, 1H), 3.74 (s, 3H), 3.73 (s, 3H), 1.37 (d, 6H).

### Example 1-11

### rac-6-(3,4-Dimethylphenyl)-N-[3-hydroxy-1-(4-methoxyphenyl)propyl]-4-oxo-5H-pyrazolo[1,5-a]-pyrazine-2-carboxamide

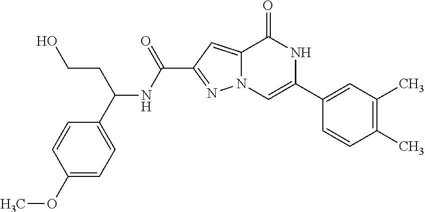

N, N-Diisopropylethylamine (92 μl, 530 μmol), N-(3-dimethylaminopropyl)-N′-ethylcarbodiimide hydrochloride (40.6 mg, 212 μmol) and 1-hydroxy-7-azabenzotriazole (150 μl, 0.60 M solution in DMF, 88 μmol) were added to a solution of 6-(3,4-dimethylphenyl)-4-oxo-4,5-dihydropyrazolo-[1,5-a]pyrazine-2-carboxylic acid (Intermediate 23A, 50.0 mg, 176 μmol) in DMF (2.5 ml), and the mixture was stirred at 50° C. for 30 min. Then, 3-amino-3-(4-methoxyphenyl)propan-1-ol (32.0 mg, 176 μmol) was added, and stirring was continued at 50° C. for 2 h. After cooling to room temperature, the reaction mixture was diluted with water and acetonitrile and directly purified by preparative HPLC (Method P1) to afford the title compound. Yield: 25.1 mg (32% of theory).

LC/MS [Method 7]: Rₜ=0.87 min; MS (ESIpos): m/z=447 [M+H]⁺.

¹H-NMR (400 MHz, DMSO-d₆): δ [ppm]=11.60 (br. s, 1H), 8.74 (d, 1H), 7.96 (s, 1H), 7.56 (s, 1H), 7.48 (d, 1H), 7.34-7.29 (m, 3H), 7.25 (d, 1H), 6.88 (d, 2H), 5.16-5.10 (m, 1H), 4.60-4.55 (m, 1H), 3.73 (s, 3H), 3.45-3.34 (m, 2H), 2.29 (s, 3H), 2.27 (s, 3H), 2.09-2.01 (m, 1H), 1.96-1.87 (m, 1H).

### Example 1-12

### rac-6-(3,4-Dimethylphenyl)-4-oxo-N-[1-phenyl-3-(1,2,4-triazol-1-yl)propyl]-5H-pyrazolo[1,5-a]-pyrazine-2-carboxamide

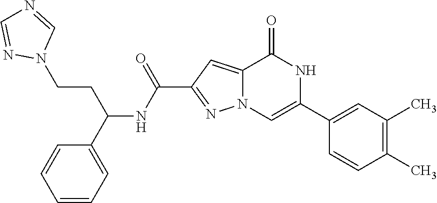 [SMILES: Cc1ccc(-c2cn3nc(C(=O)NC(CCn4cncn4)c4ccccc4)cc3c(=O)[nH]2)cc1C]

The title compound was prepared according to general procedure 1-B starting from 1-phenyl-3-(1H-1,2,4-triazol-1-yl)propan-1-amine (71.4 mg, 353 μmol), 6-(3,4-dimethylphenyl)-4-oxo-4,5-dihydropyrazolo[1,5-a]pyrazine-2-carboxylic acid (Intermediate 23A, 100 mg, 353 μmol), DMAP (21.6 mg, 176 μmol) and CDI (114 mg, 706 μmol) in NMP (5.0 ml). The reaction mixture was stirred at 50° C. for 6 h, then cooled to RT and directly purified by preparative HPLC (Method P1) to afford the title compound. Yield: 99.1 mg (60% of theory).

LC/MS [Method 3]: Rₜ=1.62 min; MS (ESIpos): m/z=468 [M+H]⁺.

¹H-NMR (400 MHz, DMSO-d₆): δ [ppm]=11.66 (s, 1H), 9.02 (d, 1H), 8.47 (s, 1H), 7.99 (s, 1H), 7.95 (s, 1H), 7.56 (s, 1H), 7.47 (d, 1H), 7.43-7.32 (m, 5H), 7.26 (d, 2H), 5.03-4.96 (m, 1H), 4.29-4.19 (m, 2H), 2.41-2.28 (m, 5H), 2.28 (s, 3H).

### Example 1-13

### 6-(3,4-Dimethylphenyl)-N-[(1S)-2-methoxy-1-phenylethyl]-4-oxo-4,5-dihydropyrazolo[1,5-a]-pyrazine-2-carboxamide

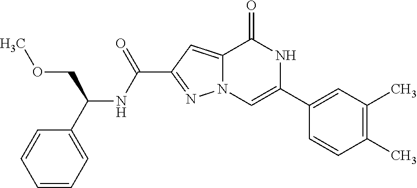 [SMILES: COC[C@@H](NC(=O)c1cc2c(=O)[nH]c(-c3ccc(C)c(C)c3)cn2n1)c1ccccc1]

The title compound was prepared according to general procedure 1-B starting from (1S)-2-methoxy-1-phenylethanamine (53.4 mg, 353 μmol), 6-(3,4-dimethylphenyl)-4-oxo-4,5-dihydropyrazolo[1,5-a]pyrazine-2-carboxylic acid (Intermediate 23A, 100 mg, 353 μmol), CDI (114 mg, 706 μmol) and DMAP (21.6 mg, 176 μmol) in NMP (5.0 ml). After cooling to RT, the reaction mixture was directly purified by preparative HPLC (Method P1) to afford the title compound. Yield: 47.7 mg (32% of theory).

LC/MS [Method 3]: Rₜ=1.88 min; MS (ESIpos): m/z=417 [M+H]⁺.

¹H-NMR (400 MHz, DMSO-d₆): δ [ppm]=11.65 (br. s, 1H), 8.72 (d, 1H), 8.00 (s, 1H), 7.57 (s, 1H), 7.50-7.46 (m, 1H), 7.46-7.41 (m, 2H), 7.37-7.30 (m, 3H), 7.30-7.24 (m, 2H), 5.29-5.23 (m, 1H), 3.76 (dd, 1H), 3.60 (dd, 1H), 3.30 (s, 3H), 2.29 (s, 3H), 2.28 (s, 3H).

### Example 1-14

### 6-(3,4-Dimethylphenyl)-N-[(1R)-3-hydroxy-1-phenylpropyl]-4-oxo-4,5-dihydropyrazolo[1,5-a]-pyrazine-2-carboxamide

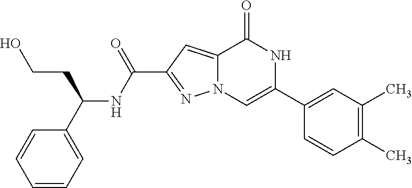 [SMILES: Cc1ccc(-c2cn3nc(C(=O)N[C@H](CCO)c4ccccc4)cc3c(=O)[nH]2)cc1C]

The title compound was prepared according to general procedure 1-B starting from 6-(3,4-dimethylphenyl)-4-oxo-4,5-dihydropyrazolo[1,5-a]pyrazine-2-carboxylic acid (Intermediate 23A, 100 mg, 353 μmol), (3R)-3-amino-3-phenylpropan-1-ol (58.7 mg, 388 μmol), CDI (63.0 mg, 388 μmol) and DMAP (21.6 mg, 176 μmol) in NMP (3.0 ml). After cooling to RT, the reaction mixture was directly purified by preparative HPLC (Method P2) to afford the title compound. Yield: 50.4 mg (34% of theory).

LC/MS [Method 3]: Rₜ=1.64 min; MS (ESIpos): m/z=417 [M+H]⁺.

¹H-NMR (400 MHz, DMSO-d₆): δ [ppm]=11.65 (s, 1H), 8.83 (d, 1H), 7.97 (s, 1H), 7.56 (s, 1H), 7.48 (dd, 1H), 7.42-7.37 (m, 2H), 7.37-7.29 (m, 3H), 7.29-7.21 (m, 2H), 5.22-5.15 (m, 1H), 4.61 (br. s, 1H), 3.47-3.38 (m, 2H), 2.29 (s, 3H), 2.27 (s, 3H), 2.13-2.03 (m, 1H), 1.98-1.89 (m, 1H).

### Example 1-15

### 6-(3,4-Dimethylphenyl)-4-oxo-N-{(1R)-1-[4-(trifluoromethyl)phenyl]ethyl}-4,5-dihydropyrazolo-[1,5-a]pyrazine-2-carboxamide

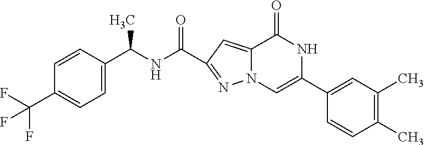 [SMILES: Cc1ccc(-c2cn3nc(C(=O)N[C@H](C)c4ccc(C(F)(F)F)cc4)cc3c(=O)[nH]2)cc1C]

The title compound was prepared according to general procedure 1-B starting from 6-(3,4-dimethylphenyl)-4-oxo-4,5-dihydropyrazolo[1,5-a]pyrazine-2-carboxylic acid (Intermediate 23A, 95.0 mg, 335 μmol), (1R)-1-[4-(trifluoromethyl)phenyl]ethanamine (63.4 mg, 335 μmol), CDI (109 mg, 671 μmol) and DMAP (20.5 mg, 168 μmol) in NMP (2.0 ml). After cooling to RT, the reaction mixture was directly purified by preparative HPLC (Method P2) to afford the title compound. Yield: 84.2 mg (55% of theory).

LC/MS [Method 11]: Rₜ=3.59 min; MS (ESIpos): m/z=455 [M+H]⁺.

¹H-NMR (400 MHz, DMSO-d₆): δ [ppm]=11.66 (s, 1H), 8.97 (d, 1H), 7.95 (s, 1H), 7.73-7.67 (m, 2H), 7.67-7.61 (m, 2H), 7.55 (s, 1H), 7.47 (dd, 1H), 7.38 (s, 1H), 7.26 (d, 1H), 5.28-5.19 (m, 1H), 2.29 (s, 3H), 2.28 (s, 3H), 1.53 (d, 3H).

### Example 1-16

### N-(3,4-Dimethoxybenzyl)-6-(4-methyl-3,4-dihydro-2H-1,4-benzoxazin-6-yl)-4-oxo-4,5-dihydropyrazolo[1,5-a]pyrazine-2-carboxamide

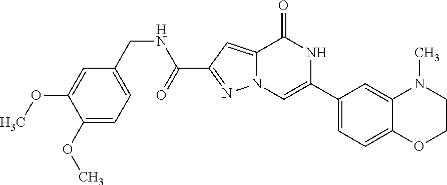 [SMILES: COc1ccc(CNC(=O)c2cc3c(=O)[nH]c(-c4ccc5c(c4)N(C)CCO5)cn3n2)cc1OC]

N,N-Diisopropylethylamine (180 μl, 1.0 mmol), HATU (141 mg, 371 μmol) and 1-(3,4-dimethoxyphenyl)methanamine (56.4 mg, 337 μmol) were added to a solution of 6-(4-methyl-3,4-dihydro-2H-1,4-benzoxazin-6-yl)-4-oxo-4,5-dihydropyrazolo[1,5-a]pyrazine-2-carboxylic acid (Intermediate 31A, 200 mg, 55% purity, 337 μmol) in DMF (8.1 ml). The mixture was stirred at RT overnight. More 1-(3,4-dimethoxyphenyl)methanamine (11.3 mg, 67.4 μmol) was added, and stirring at RT was continued for three days. Additional 1-(3,4-dimethoxyphenyl)methanamine (28.2 mg, 169 μmol) and HATU (70.5 mg, 186 μmol) were added, and stirring at RT was continued for 4 h. Then, the reaction mixture was diluted with water and extracted with dichloromethane. The combined organic layers were washed with brine, dried over sodium sulfate and filtered. The filtrate was evaporated, and the residue was purified by preparative HPLC (Method P13) to afford the title compound. Yield: 127 mg (79% of theory).

LC/MS [Method 34]: Rₜ=1.01 min; MS (ESIpos): m/z=476 [M+H]⁺.

¹H-NMR (400 MHz, DMSO-d₆): δ [ppm]=11.63 (br. s, 1H), 8.88 (t, 1H), 7.90 (d, 1H), 7.34 (d, 1H), 7.03 (d, 1H), 6.98-6.94 (m, 2H), 6.91-6.87 (m, 1H), 6.87-6.83 (m, 1H), 6.75 (d, 1H), 4.40 (d, 2H), 4.31-4.22 (m, 2H), 3.73 (s, 3H), 3.72 (s, 3H), 3.29-3.24 (m, 2H), 2.92 (s, 3H).

### Example 1-17

### N-[(1R)-1-(4-Fluorophenyl)-3-hydroxpropyl]-6-(naphthalen-2-yl)-4-oxo-3-(trifluoromethyl)-4,5-dihydropyrazolo[1,5-a]pyrazine-2-carboxamide

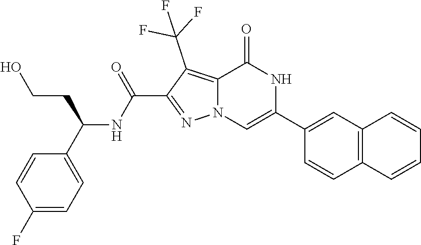 [SMILES: O=C(N[C@H](CCO)c1ccc(F)cc1)c1nn2cc(-c3ccc4ccccc4c3)[nH]c(=O)c2c1C(F)(F)F]

The title compound was prepared according to general procedure 1-B starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), (3R)-3-amino-3-(4-fluorophenyl)propan-1-ol (49.9 mg, 295 μmol), CDI (47.8 mg, 295 μmol) and DMAP (16.4 mg, 134 μmol) in N-methylpyrrolidone (2.0 ml). After cooling to RT, the reaction mixture was directly purified by preparative HPLC (Method P2) to afford the title compound. Yield: 29.0 mg (20% of theory).

LC/MS [Method 7]: Rₜ=0.96 min; MS (ESIneg): m/z=523 [M−H]⁻.

¹H-NMR (400 MHz, DMSO-d₆): δ [ppm]=12.23 (s, 1H), 9.24 (d, 1H), 8.40 (d, 2H), 8.05 (d, 1H), 8.02-7.97 (m, 2H), 7.90 (dd, 1H), 7.64-7.59 (m, 2H), 7.44-7.39 (m, 2H), 7.22-7.16 (m, 2H), 5.19-5.12 (m, 1H), 4.58 (br. s, 1H), 3.49-3.36 (m, 2H), 2.03-1.94 (m, 1H), 1.90-1.82 (m, 1H).

### Example 1-18

### N-[(1S)-2-Hydroxy-2-methyl-1-phenylpropyl]-6-(naphthalen-2-yl)-4-oxo-3-(trifluoromethyl)-4,5-dihydropyrazolo[1,5-a]pyrazine-2-carboxamide

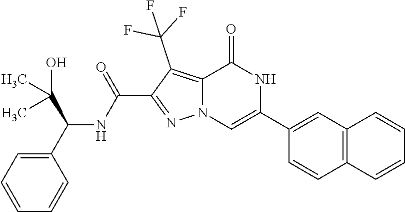 [SMILES: CC(C)(O)[C@@H](NC(=O)c1nn2cc(-c3ccc4ccccc4c3)[nH]c(=O)c2c1C(F)(F)F)c1ccccc1]

The title compound was prepared according to general procedure 1-B starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), (1S)-1-amino-2-methyl-1-phenylpropan-2-ol (Intermediate 4A, 48.7 mg, 295 μmol), CDI (47.8 mg, 295 μmol) and DMAP (16.4 mg, 134 μmol) in N-methylpyrrolidone (2.5 ml). After cooling to RT, the reaction mixture was directly purified by preparative HPLC (Method P2) to afford the title compound. Yield: 69.8 mg (50% of theory).

LC/MS [Method 7]: Rₜ=1.02 min; MS (ESIpos): m/z=521 [M+H]⁺.

¹H-NMR (400 MHz, DMSO-d₆): δ [ppm]=12.21 (s, 1H), 8.89 (d, 1H), 8.43 (dd, 2H), 8.07-8.02 (m, 1H), 8.02-7.97 (m, 2H), 7.95-7.89 (m, 1H), 7.64-7.58 (m, 2H), 7.42 (d, 2H), 7.35-7.29 (m, 2H), 7.29-7.23 (m, 1H), 4.93 (d, 1H), 4.61 (br. s, 1H), 1.20 (s, 3H), 1.06 (s, 3H).

### Example 1-19

### N-[(1S)-1-(4-Fluorophenyl)-2-hydroxy-2-methylpropyl]-6-(naphthalen-2-yl)-4-oxo-3-(trifluoromethyl)-4,5-dihydropyrazolo[1,5-a]pyrazine-2-carboxamide

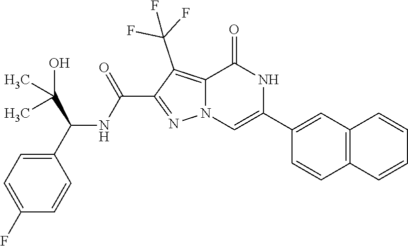 [SMILES: CC(C)(O)[C@@H](NC(=O)c1nn2cc(-c3ccc4ccccc4c3)[nH]c(=O)c2c1C(F)(F)F)c1ccc(F)cc1]

The title compound was prepared according to general procedure 1-A starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), (1S)-1-amino-1-(4-fluorophenyl)-2-methylpropan-2-ol (Intermediate 36A, 58.9 mg, 321 μmol), N,N-diisopropylethylamine (140 μl, 800 μmol) and HATU (122 mg, 321 μmol) in DMF (2.3 ml). After cooling to RT, the reaction mixture was directly purified by preparative HPLC (Method P2) to afford the title compound. Yield: 47.6 mg (32% of theory).

LC/MS [Method 7]: Rₜ=1.95 min; MS (ESIneg): m/z=537 [M−H]⁻.

¹H-NMR (600 MHz, DMSO-d₆): δ [ppm]=12.20 (s, 1H), 8.91 (d, 1H), 8.43 (s, 2H), 8.04 (d, 1H), 8.01-7.97 (m, 2H), 7.91 (dd, 1H), 7.65-7.58 (m, 2H), 7.48-7.43 (m, 2H), 7.17-7.12 (m, 2H), 4.95 (d, 1H), 4.63 (br. s, 1H), 1.20 (s, 3H), 1.05 (s, 3H).

### Example 1-20

### N-[(1R)-1-(4-Fluorophenyl)ethyl]-3-(methoxymethyl)-6-(naphthalen-2-yl)-4-oxo-4,5-dihydropyrazolo[1,5-a]pyrazine-2-carboxamide

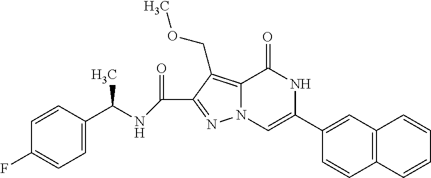 [SMILES: COCc1c(C(=O)N[C@H](C)c2ccc(F)cc2)nn2cc(-c3ccc4ccccc4c3)[nH]c(=O)c12]

3-(Methoxymethyl)-6-(naphthalen-2-yl)-4-oxo-4,5-dihydropyrazolo[1,5-a]pyrazine-2-carboxylic acid (Intermediate 42A, 120 mg, 343 μmol) was suspended in DMF (4.4 ml), and N,N-diisopropylethylamine (180 μl, 1.0 mmol), 1-(3-dimethylaminopropyl)-3-ethylcarbodiimide hydrochloride (72.4 mg, 378 μmol) and 1-hydroxy-7-azabenzotriazole (340 μl, 0.50 M solution in DMF, 170 μmol) were added. The mixture was stirred at RT for 90 min before (1R)-1-(4-fluorophenyl)ethanamine (46 μl, 340 μmol) was added. The mixture was stirred at RT overnight and then poured into water. The precipitate was collected by filtration. The filtrate was extracted with dichloromethane, and the combined organic layers were dried over magnesium sulfate, filtered and evaporated. The residue and the previously collected precipitate were combined, dissolved in dichloromethane and methanol and purified by column chromatography on silica gel (10 g silica gel, eluent: gradient of ethyl acetate in cyclohexane) to afford the title compound. Yield: 63.5 mg (39% of theory).

LC/MS [Method 7]: Rₜ=1.07 min; MS (ESIpos): m/z=471 [M+H]⁺.

¹H-NMR (400 MHz, DMSO-d₆): δ [ppm]=11.71 (br. s, 1H), 8.76 (d, 1H), 8.38 (s, 1H), 8.17 (s, 1H), 8.04 (d, 1H), 8.01-7.95 (m, 2H), 7.88 (dd, 1H), 7.65-7.55 (m, 2H), 7.51-7.42 (m, 2H), 7.20-7.13 (m, 2H), 5.21-5.13 (m, 1H), 4.97-4.88 (m, 2H), 3.23 (s, 3H), 1.50 (d, 3H).

### Example 1-21

### N-[3-(4-Fluorophenyl)-1-methylazetidin-3-yl]-3-(methoxymethyl)-6-(naphthalen-2-yl)-4-oxo-4,5-dihydropyrazolo[1,5-a]pyrazine-2-carboxamide

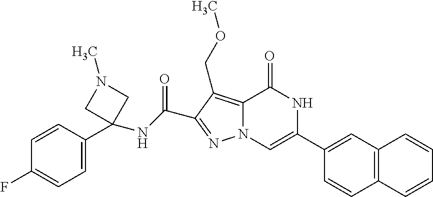 [SMILES: COCc1c(C(=O)NC2(c3ccc(F)cc3)CN(C)C2)nn2cc(-c3ccc4ccccc4c3)[nH]c(=O)c12]

The title compound was prepared according to general procedure 1-B starting from 3-(methoxymethyl)-6-(naphthalen-2-yl)-4-oxo-4,5-dihydropyrazolo[1,5-a]pyrazine-2-carboxylic acid (Intermediate 42A, 100 mg, 286 μmol), 3-(4-fluorophenyl)-1-methylazetidin-3-amine (Intermediate 46A, 51.6 mg, 286 μmol), CDI (92.8 mg, 572 μmol) and DMAP (17.5 mg, 143 μmol) in N-methyl-pyrrolidone (2.0 ml). After cooling to RT, the reaction mixture was directly purified by preparative HPLC (Method P2). The product-containing fractions were combined and evaporated. The residue was further purified by column chromatography on basic silica gel (eluent: gradient of methanol in dichloromethane) to afford the title compound. Yield: 11.0 mg (8% of theory).

LC/MS [Method 3]: Rₜ=1.26 min; MS (ESIpos): m/z=512 [M+H]⁺.

¹H-NMR (400 MHz, DMSO-d₆): δ [ppm]=12.05 (br. s, 1H), 9.38 (s, 1H), 8.40-8.37 (m, 1H), 8.14 (s, 1H), 8.04 (d, 1H), 8.02-7.97 (m, 2H), 7.88 (dd, 1H), 7.68-7.58 (m, 4H), 7.20-7.14 (m, 2H), 4.89 (s, 2H), 3.71 (d, 2H), 3.46 (d, 2H), 3.23 (s, 3H), 2.34 (s, 3H).

### Example 1-22

### 3-(Methoxymethyl)-N-[(1R)-1-(6-methoxypyridin-3-yl)ethyl]-6-(naphthalen-2-yl)-4-oxo-4,5-dihydropyrazolo[1,5-a]pyrazine-2-carboxamide

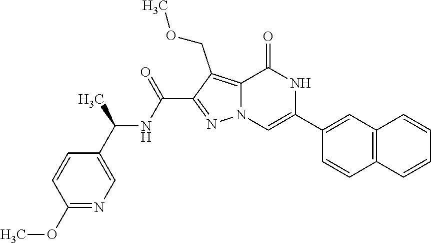 [SMILES: COCc1c(C(=O)N[C@H](C)c2ccc(OC)nc2)nn2cc(-c3ccc4ccccc4c3)[nH]c(=O)c12]

The title compound was prepared according to general procedure 1-B starting from 3-(methoxymethyl)-6-(naphthalen-2-yl)-4-oxo-4,5-dihydropyrazolo[1,5-a]pyrazine-2-carboxylic acid (Intermediate 42A, 100 mg, 286 μmol), (1R)-1-(6-methoxypyridin-3-yl)ethanamine (Intermediate 49A, 47.9 mg, 315 μmol), CDI (51.1 mg, 315 μmol) and DMAP (17.5 mg, 143 μmol) in N-methylpyrrolidone (2.4 ml). After cooling to RT, the reaction mixture was directly purified by preparative HPLC (Method P2) to afford the title compound. Yield: 97.5 mg (69% of theory).

LC/MS [Method 7]: Rₜ=0.96 min; MS (ESIpos): m/z=484 [M+H]⁺.

¹H-NMR (400 MHz, DMSO-d₆): δ [ppm]=11.84 (s, 1H), 8.78 (d, 1H), 8.38 (s, 1H), 8.21 (d, 1H), 8.16 (s, 1H), 8.04 (d, 1H), 8.01-7.96 (m, 2H), 7.87 (dd, 1H), 7.80 (dd, 1H), 7.63-7.58 (m, 2H), 6.81 (d, 1H), 5.20-5.11 (m, 1H), 4.96-4.89 (m, 2H), 3.83 (s, 3H), 3.23 (s, 3H), 1.51 (d, 3H).

### Example 1-23

### N-[(1R)-1-(4-Fluorophenyl)ethyl]-3,7-dimethyl-6-(naphthalen-2-yl)-4-oxo-4,5-dihydropyrazolo-[1,5-a]pyrazine-2-carboxamide

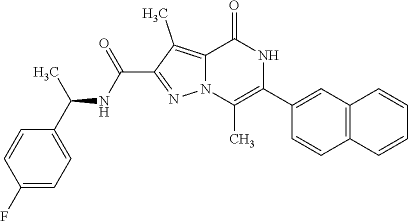 [SMILES: Cc1c(C(=O)N[C@H](C)c2ccc(F)cc2)nn2c(C)c(-c3ccc4ccccc4c3)[nH]c(=O)c12]

The title compound was prepared according to general procedure 1-B starting from 3,7-dimethyl-6-(naphthalen-2-yl)-4-oxo-4,5-dihydropyrazolo[1,5-a]pyrazine-2-carboxylic acid (Intermediate 57A, 250 mg, 750 μmol), (1R)-1-(4-fluorophenyl)ethanamine (100 μl, 750 μmol), CDI (243 mg, 1.50 mmol) and DMAP (45.8 mg, 375 μmol) in N-methylpyrrolidone (8.0 ml). The mixture was stirred at 50° C. for three days. After cooling to RT, the reaction mixture was directly purified by preparative HPLC (Method P1) to afford the title compound. Yield: 180 mg (50% of theory).

LC/MS [Method 3]: Rₜ=1.16 min; MS (ESIpos): m/z=455 [M+H]⁺.

¹H-NMR (400 MHz, DMSO-d₆): δ [ppm]=11.43 (s, 1H), 8.55 (d, 1H), 8.11-7.99 (m, 4H), 7.65-7.58 (m, 3H), 7.51-7.44 (m, 2H), 7.20-7.13 (m, 2H), 5.26-5.17 (m, 1H), 2.64 (s, 3H), 2.46-2.40 (m, 3H), 1.52 (d, 3H).

### Example 1-24

### 6-(3,4-Dihydro-1H-isochromen-7-yl)-N-[(1R)-1-(4-fluorophenyl)-3-hydroxypropyl]-4-oxo-4,5-dihydropyrazolo[1,5-a]pyrazine-2-carboxamide

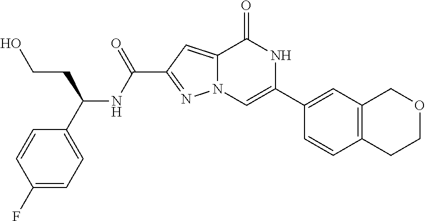 [SMILES: O=C(N[C@H](CCO)c1ccc(F)cc1)c1cc2c(=O)[nH]c(-c3ccc4c(c3)COCC4)cn2n1]

The title compound was prepared according to general procedure 1-B starting from 6-(3,4-dihydro-1H-isochromen-7-yl)-4-oxo-4,5-dihydropyrazolo[1,5-a]pyrazine-2-carboxylic acid (Intermediate 66A, 100 mg, 321 μmol), (3R)-3-amino-3-(4-fluorophenyl)propan-1-ol (59.8 mg, 353 μmol), CDI (57.3 mg, 353 μmol) and DMAP (19.6 mg, 161 μmol) in N-methylpyrrolidone (4.6 ml, 47 mmol). After cooling to RT, the reaction mixture was directly purified by preparative HPLC (Method P2) to afford the title compound. Yield: 64.8 mg (44% of theory).

LC/MS [Method 3]: Rₜ=1.44 min; MS (ESIpos): m/z=463 [M+H]⁺.

¹H-NMR (400 MHz, DMSO-d₆): δ [ppm]=11.69 (s, 1H), 8.88 (d, 1H), 7.97 (s, 1H), 7.54 (d, 1H), 7.47-7.41 (m, 3H), 7.34 (s, 1H), 7.26 (d, 1H), 7.18-7.12 (m, 2H), 5.22-5.15 (m, 1H), 4.73 (s, 2H), 4.54 (br. s, 1H), 3.91 (t, 2H), 3.48-3.39 (m, 2H), 2.83 (t, 2H), 2.12-2.02 (m, 1H), 1.97-1.87 (m, 1H).

### Example 1-25

### N-[3-(4-Fluorophenyl)-1-methylazetidin-3-yl]-6-(5-methylquinolin-3-yl)-4-oxo-3-(propan-2-yl)-4,5-dihydropyrazolo[1,5-a]pyrazine-2-carboxamide

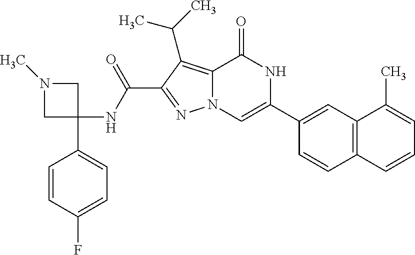 [SMILES: Cc1cccc2ccc(-c3cn4nc(C(=O)NC5(c6ccc(F)cc6)CN(C)C5)c(C(C)C)c4c(=O)[nH]3)cc12]

The title compound was prepared according to general procedure 1-A starting from 6-(5-methylquinolin-3-yl)-4-oxo-3-(propan-2-yl)-4,5-dihydropyrazolo[1,5-a]pyrazine-2-carboxylic acid (Intermediate 77A, 100 mg, 276 μmol), 3-(4-fluorophenyl)-1-methylazetidin-3-amine (Intermediate 46A, 54.7 mg, 304 μmol), HATU (126 mg, 331 μmol) and N,N-diisopropylethylamine (140 μl, 830 μmol) in N-methylpyrrolidone (4.5 ml). After cooling to RT, the reaction mixture was filtered, and the filtrate was purified by preparative HPLC (Method P3) to afford the title compound. Yield: 32.0 mg (21% of theory).

LC/MS [Method 3]: Rₜ=1.37 min; MS (ESIneg): m/z=523 [M−H]⁻.

¹H-NMR (400 MHz, DMSO-d₆): δ [ppm]=11.89 (br. s, 1H), 9.38 (s, 1H), 9.26 (d, 1H), 8.84 (d, 1H), 8.35 (s, 1H), 7.91 (d, 1H), 7.74-7.61 (m, 3H), 7.52 (d, 1H), 7.22-7.13 (m, 2H), 4.08-3.98 (m, 1H), 3.70 (d, 2H), 3.44 (d, 2H), 2.77 (s, 3H), 2.33 (s, 3H), 1.32 (d, 6H).

### Example 1-26

### 3-Cyclopropyl-N-[2-[ethyl(2-hydroxyethyl)amino]-1-phenylethyl]-6-(2-naphthyl)-4-oxo-5H-pyrazolo[1,5-a]pyrazine-2-carboxamide

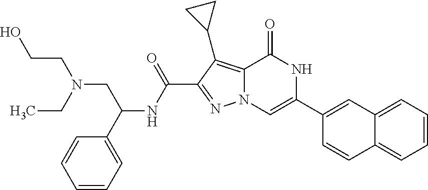 [SMILES: CCN(CCO)CC(NC(=O)c1nn2cc(-c3ccc4ccccc4c3)[nH]c(=O)c2c1C1CC1)c1ccccc1]

The title compound was prepared according to general procedure 1-A starting from 3-cyclopropyl-6-(naphthalen-2-yl)-4-oxo-4,5-dihydropyrazolo[1,5-a]pyrazine-2-carboxylic acid (Intermediate 82A, 75.0 mg, 217 μmol), (rac)-2-[(2-amino-2-phenylethyl)ethylamino]ethanol (Intermediate 85A, 49.8 mg, 239 μmol), HATU (99.1 mg, 261 μmol) and N,N-diisopropylethylamine (110 μl, 650 μmol) in N-methylpyrrolidone (2.5 ml). After cooling to RT, the reaction mixture was directly purified by preparative HPLC (Method P5). The product-containing fractions were combined, the volatiles were removed, and the residue was further purified by preparative HPLC (Method P6) to afford the title compound. Yield: 11.8 mg (10% of theory).

LC/MS [Method 3]: Rₜ=1.44 min; MS (ESIpos): m/z=536 [M+H]⁺.

¹H-NMR (400 MHz, DMSO-d₆): δ [ppm]=11.57 (br. s, 1H), 8.60 (d, 1H), 8.38 (s, 1H), 8.13 (s, 1H), 8.02 (d, 1H), 8.00-7.95 (m, 2H), 7.88 (dd, 1H), 7.62-7.56 (m, 2H), 7.46-7.38 (m, 2H), 7.38-7.30 (m, 2H), 7.28-7.22 (m, 1H), 5.11-5.05 (m, 1H), 4.31 (t, 1H), 3.48-3.42 (m, 2H), 2.90-2.71 (m, 3H), 2.67-2.56 (m, 4H), 1.27-1.16 (m, 2H), 0.95 (t, 3H), 0.90-0.81 (m, 2H).

### Example 1-27

### N-[2-[Ethyl(2-hydroxyethyl)amino]-1-phenylethyl]-6-(2-naphthyl)-4-oxo-5H-pyrazolo[1,5-a]-pyrazine-2-carboxamide

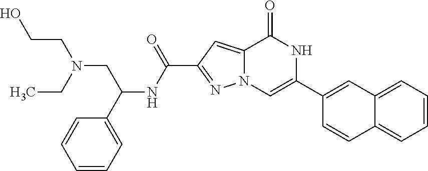 [SMILES: CCN(CCO)CC(NC(=O)c1cc2c(=O)[nH]c(-c3ccc4ccccc4c3)cn2n1)c1ccccc1]

The title compound was prepared according to general procedure 1-A starting from 6-(naphthalen-2-yl)-4-oxo-4,5-dihydropyrazolo[1,5-a]pyrazine-2-carboxylic acid (Intermediate 3A, 80.0 mg, 262 μmol), (rac)-2-[(2-amino-2-phenylethyl)ethylamino]ethanol (Intermediate 85A, 60.0 mg, 288 μmol), HATU (120 mg, 314 μmol) and N,N-diisopropylethylamine (140 μl, 790 μmol) in N-methylpyrrolidone (3.0 ml). After cooling to RT, the reaction mixture was diluted with DMSO (5.0 ml) and directly purified by preparative HPLC (Method P5). The product-containing fractions were combined, the volatiles were removed, and the residue was further purified by preparative HPLC (Method P7) to afford the title compound. Yield: 25.0 mg (19% of theory).

LC/MS [Method 3]: Rₜ=1.26 min; MS (ESIpos): m/z=496 [M+H]⁺.

¹H-NMR (400 MHz, DMSO-d₆): δ [ppm]=11.80 (br. s, 1H), 8.67 (d, 1H), 8.39 (s, 1H), 8.22 (s, 1H), 8.04 (d, 1H), 8.02-7.96 (m, 2H), 7.89 (dd, 1H), 7.63-7.58 (m, 2H), 7.46-7.36 (m, 3H), 7.36-7.28 (m, 2H), 7.28-7.22 (m, 1H), 5.14-5.07 (m, 1H), 4.34 (t, 1H), 3.51-3.40 (m, 2H), 2.96-2.86 (m, 1H), 2.84-2.76 (m, 1H), 2.71-2.57 (m, 4H), 0.94 (t, 3H).

### Example 1-28

### N-[2-[Ethyl(2-hydroxyethyl)amino]-1-phenylethyl]-6-(2-naphthyl)-4-oxo-3-(trifluoromethyl)-5H-pyrazolo[1,5-a]pyrazine-2-carboxamide

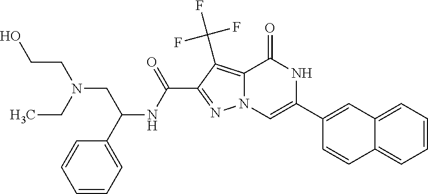 [SMILES: CCN(CCO)CC(NC(=O)c1nn2cc(-c3ccc4ccccc4c3)[nH]c(=O)c2c1C(F)(F)F)c1ccccc1]

The title compound was prepared according to general procedure 1-A starting from 6-(naphthalen-2-yl)-4-oxo-3-(trifluoromethyl)-4,5-dihydropyrazolo[1,5-a]pyrazine-2-carboxylic acid (Intermediate 35A, 80.0 mg, 214 μmol), (rac)-2-[(2-amino-2-phenylethyl)ethylamino]ethanol (Intermediate 85A, 49.1 mg, 236 μmol), HATU (97.8 mg, 257 μmol) and N,N-diisopropylethylamine (110 μl, 640 μmol) in N-methylpyrrolidone (2.5 ml). After cooling to RT, the reaction mixture was diluted with DMSO (5.0 ml) and directly purified by preparative HPLC (Method P5). The product-containing fractions were combined, the volatiles were removed, and the residue was further purified by preparative HPLC (Method P8) to afford the title compound. Yield: 30.0 mg (25% of theory).

LC/MS [Method 7]: Rₜ=0.76 min; MS (ESIpos): m/z=564 [M+H]⁺.

¹H-NMR (400 MHz, DMSO-d₆): δ [ppm]=11.99 (br. s, 1H), 9.09 (d, 1H), 8.43 (s, 1H), 8.37 (s, 1H), 8.04 (d, 1H), 8.01-7.95 (m, 2H), 7.92 (dd, 1H), 7.65-7.55 (m, 2H), 7.44-7.38 (m, 2H), 7.38-7.32 (m, 2H), 7.30-7.20 (m, 1H), 5.14-5.07 (m, 1H), 4.27 (t, 1H), 3.46-3.39 (m, 2H), 2.85-2.70 (m, 2H), 2.64-2.56 (m, 4H), 0.93 (t, 3H).

### Example 1-29

### N-[(1S)-1-(4-Fluorophenyl)-2-hydroxy-2-methylpropyl]-6-(naphthalen-2-yl)-4-oxo-4,5-dihydropyrazolo[1,5-a]pyrazine-2-carboxamide

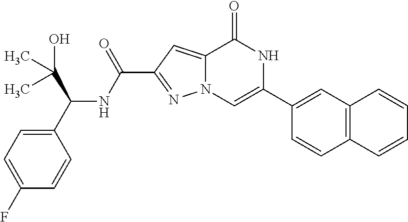 [SMILES: CC(C)(O)[C@@H](NC(=O)c1cc2c(=O)[nH]c(-c3ccc4ccccc4c3)cn2n1)c1ccc(F)cc1]

to The title compound was prepared according to general procedure 1-A starting from 6-(naphthalen-2-yl)-4-oxo-4,5-dihydropyrazolo[1,5-a]pyrazine-2-carboxylic acid (Intermediate 3A, 100 mg, 328 μmol), (1S)-1-amino-1-(4-fluorophenyl)-2-methylpropan-2-ol (Intermediate 36A, 72.0 mg, 393 μmol), HATU (149 mg, 393 μmol) and N,N-diisopropylethylamine (170 μl, 980 μmol) in DMF (2.8 ml). After cooling to RT, the reaction mixture was poured into water. The precipitate was filtered off, washed with water and dried. The obtained solid was purified by column chromatography on basic silica gel (eluent: gradient of methanol in dichloromethane). Yield: 38.5 mg (25% of theory).

LC/MS [Method 3]: Rₜ=1.81 min; MS (ESIpos): m/z=471 [M+H]⁺.

¹H-NMR (400 MHz, DMSO-d₆): δ [ppm]=11.88 (s, 1H), 8.41 (s, 1H), 8.37 (s, 1H), 8.32 (d, 1H), 8.04 (d, 1H), 8.02-7.94 (m, 2H), 7.91 (dd, 1H), 7.64-7.57 (m, 2H), 7.49-7.42 (m, 2H), 7.34 (s, 1H), 7.18-7.10 (m, 2H), 5.00 (s, 1H), 4.88 (d, 1H), 1.27 (s, 3H), 0.98 (s, 3H).

### Example 1-30

### 6-(2-Naphthyl)-4-oxo-N-[1-[5-(2,2,2-trifluoroethoxy)-2-pyridyl]ethyl]-3-(trifluoromethyl)-5H-pyrazolo[1,5-a]pyrazine-2-carboxamide trifluoroacetic acid salt

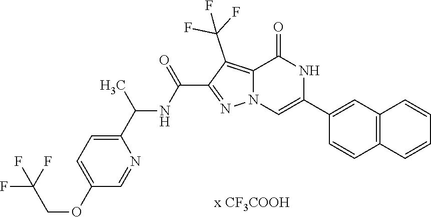 [SMILES: CC(NC(=O)c1nn2cc(-c3ccc4ccccc4c3)[nH]c(=O)c2c1C(F)(F)F)c1ccc(OCC(F)(F)F)cn1]

The title compound was prepared according to general procedure 1-A starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), 1-[5-(2,2,2-trifluoroethoxy)pyridin-2-yl]ethanamine dihydrochloride (94.2 mg, 321 μmol), HATU (122 mg, 321 μmol) and N,N-diisopropylethylamine (140 μl, 800 μmol) in N-methylpyrrolidone (2.5 ml). After cooling to RT, the reaction mixture was directly purified by preparative HPLC (Method P2). Yield: 106 mg (57% of theory).

LC/MS [Method 3]: Rₜ=2.06 min; MS (ESIpos): m/z=576 [M+H]⁺.

¹H-NMR (400 MHz, DMSO-d₆): δ [ppm]=12.23 (s, 1H), 9.18 (d, 1H), 8.43-8.39 (m, 2H), 8.37 (d, 1H), 8.05 (d, 1H), 8.03-7.97 (m, 2H), 7.90 (dd, 1H), 7.65-7.56 (m, 3H), 7.44 (d, 1H), 5.22-5.15 (m, 1H), 4.88 (q, 2H), 1.47 (d, 3H).

### Example 1-31

### N-[3-(4-Fluorophenyl)-1-methylazetidin-3-yl]-6-(naphthalen-2-yl)-4-oxo-3-(trifluoromethyl)-5H-pyrazolo[1,5-a]pyrazine-2-carboxamide trifluoroacetic acid salt

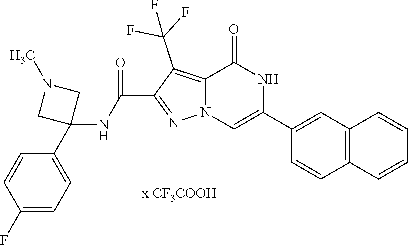 [SMILES: CN1CC(NC(=O)c2nn3cc(-c4ccc5ccccc5c4)[nH]c(=O)c3c2C(F)(F)F)(c2ccc(F)cc2)C1]

The title compound was prepared according to general procedure 1-A starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), 3-(4-fluorophenyl)-1-methylazetidin-3-amine (Intermediate 46A, 57.9 mg, 321 μmol), HATU (122 mg, 321 μmol) and N,N-diisopropylethylamine (140 μl, 800 μmol) in NMP (3.0 ml). After cooling to RT, the reaction mixture was directly purified by preparative HPLC (Method P2). Yield: 107 mg (57% of theory).

LC/MS [Method 3]: Rₜ=1.31 min; MS (ESIpos): m/z=536 [M+H]⁺.

¹H-NMR (400 MHz, DMSO-d₆): δ [ppm]=12.29 (br. s, 1H), 10.56-10.45 (m, 0.5H), 10.17-10.02 (m, 1.5H), 8.39 (s, 1H), 8.31-8.26 (m, 1H), 8.06 (d, 1H), 8.03-7.97 (m, 2H), 7.88 (dd, 1H), 7.71-7.59 (m, 3H), 7.59-7.52 (m, 1H), 7.40-7.24 (m, 2H), 4.84-4.71 (m, 2H), 4.64-4.40 (m, 2H), 3.05-2.89 (m, 3H).

### Example 1-32

### N-[3-(4-Fluorophenyl)-1-methylazetidin-3-yl]-6-(naphthalen-2-yl)-4-oxo-3-(trifluoromethyl)-4,5-dihydropyrazolo[1,5-a]pyrazine-2-carboxamide

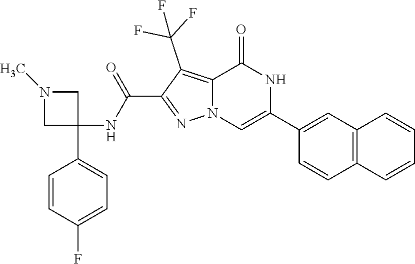 [SMILES: CN1CC(NC(=O)c2nn3cc(-c4ccc5ccccc5c4)[nH]c(=O)c3c2C(F)(F)F)(c2ccc(F)cc2)C1]

The title compound was prepared according to general procedure 1-A starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), 3-(4-fluorophenyl)-1-methylazetidin-3-amine (Intermediate 46A, 57.9 mg, 321 μmol), HATU (122 mg, 321 μmol) and N,N-diisopropylethylamine (140 μl, 800 μmol) in DMF (3.0 ml). After cooling to RT, the mixture was poured into water and extracted with dichloromethane. The combined organic layers were washed with brine, dried over magnesium sulfate and evaporated to dryness. The residue was purified by column chromatography on basic silica gel (eluent: gradient of methanol in dichloromethane). Yield: 57.5 mg (40% of theory).

LC/MS [Method 7]: Rₜ=0.75 min; MS (ESIpos): m/z=536 [M+H]⁺.

¹H-NMR (400 MHz, DMSO-d₆): δ [ppm]=12.24 (br. s, 1H), 9.77 (s, 1H), 8.43-8.39 (m, 2H), 8.05 (d, 1H), 8.03-7.97 (m, 2H), 7.90 (dd, 1H), 7.68-7.58 (m, 4H), 7.25-7.18 (m, 2H), 3.69 (d, 2H), 3.43 (d, 2H), 2.34 (s, 3H).

### Example 1-33

### N-[3-(4-Fluorophenyl)-1-methylazetidin-3-yl]-6-(naphthalen-2-yl)-4-oxo-3-(trifluoromethyl)-4,5-dihydropyrazolo[1,5-a]pyrazine-2-carboxamide hydrochloride

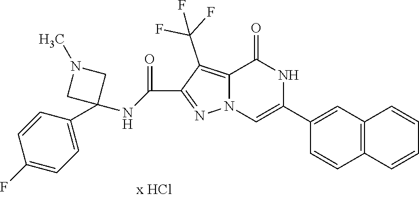 [SMILES: CN1CC(NC(=O)c2nn3cc(-c4ccc5ccccc5c4)[nH]c(=O)c3c2C(F)(F)F)(c2ccc(F)cc2)C1]

To a solution of N-[3-(4-fluorophenyl)-1-methylazetidin-3-yl]-6-(naphthalen-2-yl)-4-oxo-3-(trifluoro-methyl)-4,5-dihydropyrazolo[1,5-a]pyrazine-2-carboxamide (Example 1-32, 20.0 mg, 37.3 μmol) in acetonitrile (2.0 ml) was added hydrogen chloride (19 μl, 4.0 M solution in 1,4-dioxane, 75 μmol). The mixture was stirred at RT for 1 h and then lyophilized overnight to afford the title compound. Yield: 21.3 mg (quant.).

LC/MS [Method 7]: Rₜ=0.74 min; MS (ESIpos): m/z=536 [M+H]⁺.

¹H-NMR (400 MHz, DMSO-d₆): δ [ppm]=12.28 (br. s, 1H), 10.78-10.37 (m, 1H), 10.20-10.09 (m, 1H), 8.44-8.36 (m, 1H), 8.33-8.28 (m, 1H), 8.06 (d, 1H), 8.04-7.97 (m, 2H), 7.88 (dd, 1H), 7.72-7.65 (m, 1H), 7.65-7.59 (m, 2H), 7.59-7.52 (m, 1H), 7.37-7.26 (m, 2H), 4.83-4.71 (m, 2H), 4.57-4.41 (m, 2H), 3.04-2.84 (m, 3H).

### Example 1-34

### N-[3-(4-Fluorophenyl)-1-methylazetidin-3-yl]-3-methyl-6-(naphthalen-2-yl)-4-oxo-4,5-dihydropyrazolo[1,5-a]pyrazine-2-carboxamide formic acid salt

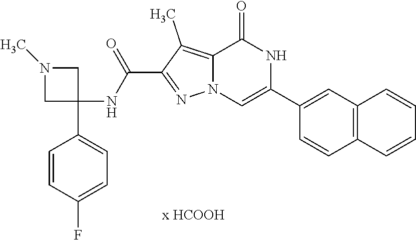 [SMILES: Cc1c(C(=O)NC2(c3ccc(F)cc3)CN(C)C2)nn2cc(-c3ccc4ccccc4c3)[nH]c(=O)c12]

To a suspension of 3-methyl-6-(naphthalen-2-yl)-4-oxo-4,5-dihydropyrazolo[1,5-a]pyrazine-2-carboxylic acid (Intermediate 88A, 200 mg, 626 μmol) were added N,N-diisopropylethylamine (330 μl, 1.9 mmol), 1-hydroxy-7-azabenzotriazole (310 μl, 1.0 M solution in DMF, 310 μmol) and N-(3-dimethylaminopropyl)-N′-ethylcarbodiimide hydrochloride (132 mg, 689 μmol). The mixture was stirred at RT for 1 h until a clear solution was obtained. 3-(4-Fluorophenyl)-1-methylazetidin-3-amine (Intermediate 46A, 113 mg, 626 μmol) was added, and stirring was continued at RT. After completion of the reaction, the reaction mixture was poured into water, and the precipitate was collected by filtration. The solid was dissolved in acetonitrile and formic acid, and the solution was purified by preparative HPLC (Method P1). Yield: 96.6 mg (28% of theory).

LC/MS [Method 3]: Rₜ=1.35 min; MS (ESIpos): m/z=482 [M+H]⁺.

¹H-NMR (400 MHz, DMSO-d₆): δ [ppm]=11.66 (br. s, 1H), 9.30 (s, 1H), 8.37 (s, 1H), 8.17 (s, 1H), 8.05-7.96 (m, 4H), 7.86 (dd, 1H), 7.67-7.57 (m, 4H), 7.20-7.14 (m, 2H), 3.74 (d, 2H), 3.50 (d, 2H), 2.59 (s, 3H), 2.35 (s, 3H).

### Example 1-35

### N-[3-(4-Fluorophenyl)-1-methylazetidin-3-yl]-3-methyl-6-(naphthalen-2-yl)-4-oxo-4,5-dihydropyrazolo[1,5-a]pyrazine-2-carboxamide hydrochloride

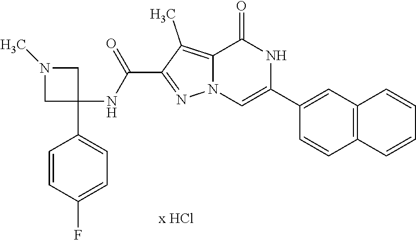 [SMILES: Cc1c(C(=O)NC2(c3ccc(F)cc3)CN(C)C2)nn2cc(-c3ccc4ccccc4c3)[nH]c(=O)c12]

N-[3-(4-Fluorophenyl)-1-methylazetidin-3-yl]-3-methyl-6-(naphthalen-2-yl)-4-oxo-4,5-dihydropyrazolo[1,5-a]pyrazine-2-carboxamide formic acid salt (Example 1-34, 40.0 mg, 75.8 μmol) was dissolved in 1,4-dioxane (15.0 ml) under gentle heating. Then, hydrogen chloride (4.0 ml, 4.0 M solution in 1,4-dioxane, 16 mmol) was added, and the mixture was stirred at RT for 4 h. The volatiles were removed, and the residue was re-dissolved in acetonitrile and water and lyophilized overnight to afford the title compound. Yield: 40.4 mg (quant.).

LC/MS [Method 3]: Rₜ=1.29 min; MS (ESIpos): m/z=482 [M+H]⁺.

¹H-NMR (400 MHz, DMSO-d₆): δ [ppm]=11.71 (s, 1H), 10.69-10.27 (m, 1H), 9.79-9.61 (m, 1H), 8.36 (s, 1H), 8.06-7.93 (m, 4H), 7.85 (dd, 1H), 7.73-7.53 (m, 4H), 7.35-7.24 (m, 2H), 4.90-4.41 (m, 4H), 2.90 (br. s, 3H), 2.61 (s, 3H).

### Example 1-36

### 3-Cyclopropyl-6-(3,4-dimethylphenyl)-N-[3-(4-fluorophenyl)-1-methylazetidin-3-yl]-4-oxo-4,5-dihydropyrazolo[1,5-a]pyrazine-2-carboxamide

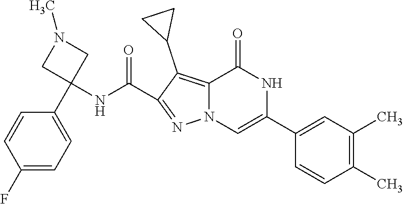 [SMILES: Cc1ccc(-c2cn3nc(C(=O)NC4(c5ccc(F)cc5)CN(C)C4)c(C4CC4)c3c(=O)[nH]2)cc1C]

The title compound was prepared according to general procedure 1-A starting from 3-cyclopropyl-6-(3,4-dimethylphenyl)-4-oxo-4,5-dihydropyrazolo[1,5-a]pyrazine-2-carboxylic acid (Intermediate 92A, 60.0 mg, 186 μmol), 3-(4-fluorophenyl)-1-methylazetidin-3-amine (Intermediate 46A, 36.8 mg, 204 μmol), HATU (84.7 mg, 223 μmol) and N,N-diisopropylethylamine (97 μl, 560 μmol) in N-methylpyrrolidone (3.0 ml). After cooling to RT, the reaction mixture was diluted with DMSO (5.0 ml) and directly purified by preparative HPLC (Method P5). The product-containing fractions were combined, the volatiles were removed, and the residue was further purified by preparative HPLC (Method P3) to afford the title compound. Yield: 11.0 mg (12% of theory).

LC/MS [Method 3]: Rₜ=1.38 min; MS (ESIpos): m/z=486 [M+H]⁺.

¹H-NMR (600 MHz, DMSO-d₆): δ [ppm]=11.34 (br. s, 1H), 9.28 (s, 1H), 7.85 (s, 1H), 7.67-7.60 (m, 2H), 7.55 (br. s, 1H), 7.46 (dd, 1H), 7.24 (d, 1H), 7.21-7.13 (m, 2H), 3.68 (d, 2H), 3.42 (d, 2H), 2.75-2.70 (m, 1H), 2.32 (s, 3H), 2.28 (s, 3H), 2.27 (s, 3H), 1.14-1.08 (m, 2H), 0.86-0.80 (m, 2H).

### Example 1-37

### N-[(1R)-1-(4-Fluorophenyl)-3-hydroxopyl]-6-(4-methyl-3,4-dihydro-2H-1,4-benzoxazin-7-yl)-4-oxo-4,5-dihydropyrazolo[1,5-a]pyrazine-2-carboxamide

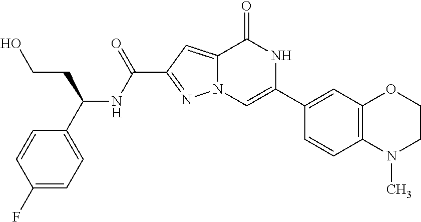 [SMILES: CN1CCOc2cc(-c3cn4nc(C(=O)N[C@H](CCO)c5ccc(F)cc5)cc4c(=O)[nH]3)ccc21]

The title compound was prepared according to general procedure 1-A starting from 6-(4-methyl-3,4-dihydro-2H-1,4-benzoxazin-7-yl)-4-oxo-4,5-dihydropyrazolo[1 ,5-a]pyrazine-2-carboxylic acid (Intermediate 98A, 75.0 mg, 230 μmol), (3R)-3-amino-3-(4-fluorophenyl)propan-1-ol (42.8 mg, 253 μmol), HATU (105 mg, 276 μmol) and N,N-diisopropylethylamine (120 μl, 690 μmol) in N-methylpyrrolidone (1.5 ml). After cooling to RT, the reaction mixture was filtered, and the filtrate was purified by preparative HPLC (Method P9) to afford the title compound. Yield: 36.0 mg (33% of theory).

LC/MS [Method 3]: Rₜ=1.53 min; MS (ESIpos): m/z=478 [M+H]⁺.

¹H-NMR (400 MHz, DMSO-d₆): δ [ppm]=11.50 (br. s, 1H), 8.84 (d, 1H), 7.83 (s, 1H), 7.47-7.40 (m, 2H), 7.29 (s, 1H), 7.21 (dd, 1H), 7.19-7.11 (m, 2H), 7.10 (d, 1H), 6.76 (d, 1H), 5.21-5.14 (m, 1H), 4.61 (t, 1H), 4.28-4.22 (m, 2H), 3.47-3.34 (m, 2H), 2.90 (s, 3H), 2.11-2.02 (m, 1H), 1.96-1.87 (m, 1H).

### Example 1-38

### N-[(1S)-2-(Dimethylamino)-1-phenylethyl]-6-(naphthalen-2-yl)-4-oxo-4,5-dihydropyrazolo[1,5-a]-pyrazine-2-carboxamide formic acid salt

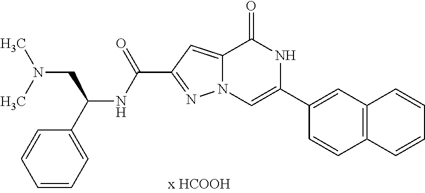 [SMILES: CN(C)C[C@@H](NC(=O)c1cc2c(=O)[nH]c(-c3ccc4ccccc4c3)cn2n1)c1ccccc1]

To a solution of 6-(naphthalen-2-yl)-4-oxo-4,5-dihydropyrazolo[1,5-a]pyrazine-2-carboxylic acid (Intermediate 3A, 200 mg, 655 μmol) in N-methylpyrrolidone (5.0 ml) were added 1-hydroxy-7-azabenzotriazole (660 μl, 0.50 M solution in DMF, 330 μmol), N-(3-dimethylaminopropyl)-N′-ethylcarbodiimide hydrochloride (138 mg, 721 μmol) and N,N-diisopropylethylamine (340 μl, 2.0 mmol). After stirring at RT for 1 h, (1S)-N²,N²-dimethyl-1-phenylethane-1,2-diamine (108 mg, 655 μmol) was added, and the mixture was heated to 50° C. overnight. After cooling to RT, the reaction mixture was directly purified by preparative HPLC (Method P1) to afford the title compound. Yield: 137 mg (42% of theory).

LC/MS [Method 7]: Rₜ=0.73 min; MS (ESIpos): m/z=452 [M+H]⁺.

¹H-NMR (400 MHz, DMSO-d₆): δ [ppm]=11.90 (br. s, 1H), 8.65 (d, 1H), 8.39 (s, 1H), 8.23 (s, 1H), 8.16 (s, 1H), 8.04 (d, 1H), 8.02-7.95 (m, 2H), 7.89 (dd, 1H), 7.64-7.57 (m, 2H), 7.45-7.41 (m, 2H), 7.39 (s, 1H), 7.37-7.31 (m, 2H), 7.28-7.22 (m, 1H), 5.20-5.12 (m, 1H), 2.96-2.82 (m, 1H), 2.25 (s, 6H).

### Example 1-39

### 6-(3,4-Dimethylphenyl)-N-[(1R)-1-(4-fluorophenyl)-3-hydroxpropyl]-4-oxo-4,5-dihydropyrazolo-[1,5-a]pyrazine-2-carboxamide

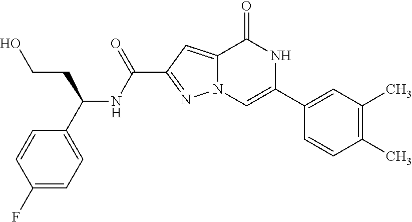 [SMILES: Cc1ccc(-c2cn3nc(C(=O)N[C@H](CCO)c4ccc(F)cc4)cc3c(=O)[nH]2)cc1C]

The title compound was prepared according to general procedure 1-A starting from 6-(3,4-dimethylphenyl)-4-oxo-4,5-dihydropyrazolo[1,5-a]pyrazine-2-carboxylic acid (Intermediate 23A, 300 mg, 1.06 mmol), (3R)-3-amino-3-(4-fluorophenyl)propan-1-ol (215 mg, 1.27 mmol), N,N-diisopropylethylamine (550 μl, 3.2 mmol) and HATU (483 mg, 1.27 mmol) in DMF (9.0 ml). After cooling to RT, the reaction mixture was poured into water (100 ml), and the precipitate was filtered off, washed with water and dried under reduced pressure. The crude product was purified by column chromatography on basic silica gel (eluent: gradient of methanol in dichloromethane). Yield: 243 mg (52% of theory).

LC/MS [Method 3]: Rₜ=1.64 min; MS (ESIpos): m/z=435 [M+H]⁺.

¹H-NMR (400 MHz, DMSO-d₆): δ [ppm]=11.63 (br. s, 1H), 8.87 (d, 1H), 7.95 (s, 1H), 7.56 (s, 1H), 7.50-7.41 (m, 3H), 7.33 (s, 1H), 7.25 (d, 1H), 7.19-7.12 (m, 2H), 5.22-5.15 (m, 1H), 4.62 (t, 1H), 3.47-3.36 (m, 2H), 2.29 (s, 3H), 2.27 (s, 3H), 2.13-2.02 (m, 1H), 1.97-1.88 (m, 1H).

### Example 1-40

### N-[1-(4-Fluorophenyl)-2-morpholinoethyl]-6-(2-naphthyl)-4-oxo-5H-pyrazolo[1,5-a]pyrazine-2-carboxamide

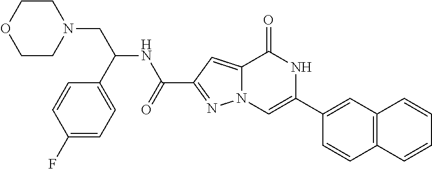 [SMILES: O=C(NC(CN1CCOCC1)c1ccc(F)cc1)c1cc2c(=O)[nH]c(-c3ccc4ccccc4c3)cn2n1]

To a solution of 6-(naphthalen-2-yl)-4-oxo-4,5-dihydropyrazolo[1,5-a]pyrazine-2-carboxylic acid (Intermediate 3A, 20.5 mg, 98% purity, 65.6 μmol) in DMF (1.0 ml) were added 1-hydroxy-7-azabenzotriazole (55 μl, 0.60 M solution in DMF, 33 μmol), N-(3-dimethylaminopropyl)-N′-ethyl-carbodiimide hydrochloride (13.8 mg, 72.2 μmol) and N,N-diisopropylethylamine (46 μl, 260 μmol). After stirring at RT for 1 h, 1-(4-fluorophenyl)-2-(morpholin-4-yl)ethanamine dihydrochloride (21.4 mg, 91% purity, 65.6 μmol) was added, and the mixture was heated to 50° C. overnight. After cooling to room temperature, the reaction mixture was poured into water and extracted with ethyl acetate. The combined organic layers were dried over sodium sulfate and filtered. The filtrate was concentrated, and the residue was dried under reduced pressure. The residue was triturated with acetonitrile, and the precipitate was filtered off, washed with acetonitrile and dried. The solid was dissolved in DMSO and the solution was purified by preparative HPLC (Method P4) to afford the title compound. Yield: 36.3 mg (quant.).

LC/MS [Method 3]: Rₜ=1.34 min; MS (ESIpos): m/z=512 [M+H]⁺.

¹H-NMR (400 MHz, DMSO-d₆): δ [ppm]=11.88 (s, 1H), 8.76 (d, 1H), 8.39 (s, 1H), 8.20 (s, 1H), 8.04 (d, 1H), 8.02-7.95 (m, 2H), 7.89 (dd, 1H), 7.64-7.57 (m, 2H), 7.52-7.45 (m, 2H), 7.39 (s, 1H), 7.19-7.13 (m, 2H), 5.27-5.19 (m, 1H), 3.60-3.48 (m, 4H), 2.94-2.84 (m, 1H).

### Example 1-41

### N-[2-[Ethyl(2-hydroxyethyl)amino]-1-phenylethyl]-3-isopropyl-6-(5-methyl-3-quinolyl)-4-oxo-5H-pyrazolo[1,5-a]pyrazine-2-carboxamide

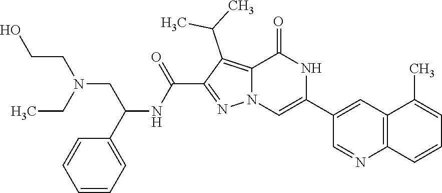 [SMILES: CCN(CCO)CC(NC(=O)c1nn2cc(-c3cnc4cccc(C)c4c3)[nH]c(=O)c2c1C(C)C)c1ccccc1]

The title compound was prepared according to general procedure 1-A starting from 6-(5-methylquinolin-3-yl)-4-oxo-3-(propan-2-yl)-4,5-dihydropyrazolo[1,5-a]pyrazine-2-carboxylic acid (Intermediate 77A, 100 mg, 276 μmol), 2-[(2-amino-2-phenylethyl)ethylamino]ethanol (Intermediate 85A, 63.2 mg, 304 μmol), HATU (126 mg, 331 μmol) and N,N-diisopropylethylamine (140 μl, 830 μmol) in N-methylpyrrolidone (4.5 ml). After cooling to RT, the reaction mixture was filtered, and the filtrate was purified by preparative HPLC (Method P3) to afford the title compound. Yield: 48.0 mg (30% of theory).

LC/MS [Method 3]: Rₜ=1.43 min; MS (ESIpos): m/z=551 [M+H]⁺.

¹H-NMR (400 MHz, DMSO-d₆): δ [ppm]=11.87 (br. s, 1H), 9.27 (d, 1H), 8.84 (d, 1H), 8.60 (d, 1H), 8.39 (s, 1H), 7.90 (d, 1H), 7.74-7.68 (m, 1H), 7.52 (d, 1H), 7.42 (d, 2H), 7.38-7.31 (m, 2H), 7.28-7.22 (m, 1H), 5.14-5.07 (m, 1H), 4.30 (t, 1H), 4.14-4.05 (m, 1H), 3.48-3.41 (m, 2H), 2.92-2.84 (m, 1H), 2.82-2.74 (m, 4H), 2.65-2.55 (m, 4H), 1.35 (d, 3H), 1.33 (d, 3H), 0.94 (t, 3H).

### Example 1-42

### N-[3-(4-Fluorophenyl)-1-methylazetidin-3-yl]-6-(4-methyl-3,4-dihydro-2H-1,4-benzoxazin-7-yl)-4-oxo-4,5-dihydropyrazolo[1,5-a]pyrazine-2-carboxamide

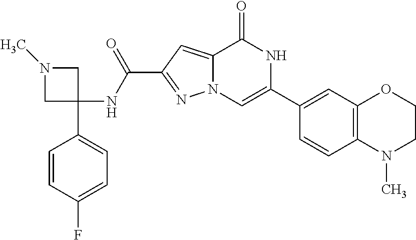 [SMILES: CN1CC(NC(=O)c2cc3c(=O)[nH]c(-c4ccc5c(c4)OCCN5C)cn3n2)(c2ccc(F)cc2)C1]

The title compound was prepared according to general procedure 1-A starting from 6-(4-methyl-3,4-dihydro-2H-1,4-benzoxazin-7-yl)-4-oxo-4,5-dihydropyrazolo[1,5-a]pyrazine-2-carboxylic acid (Intermediate 98A, 75.0 mg, 230 μmol), 3-(4-fluorophenyl)-1-methylazetidin-3-amine (Intermediate 46A, 45.6 mg, 253 μmol), HATU (105 mg, 276 μmol) and N,N-diisopropylethylamine (120 μl, 690 μmol) in N-methylpyrrolidone (1.5 ml). After cooling to RT, the reaction mixture was filtered, and the filtrate was purified by preparative HPLC (Method P7) to afford the title compound. Yield: 28.0 mg (25% of theory).

LC/MS [Method 3]: Rₜ=1.09 min; MS (ESIpos): m/z=489 [M+H]⁺.

¹H-NMR (400 MHz, DMSO-d₆): δ [ppm]=11.52 (br. s, 1H), 9.39 (s, 1H), 7.81 (s, 1H), 7.65-7.58 (m, 2H), 7.28 (d, 1H), 7.21 (dd, 1H), 7.19-7.12 (m, 2H), 7.10 (d, 1H), 6.77 (d, 1H), 4.28-4.22 (m, 2H), 3.69 (d, 2H), 3.48-3.41 (m, 2H), 2.90 (s, 3H), 2.31 (s, 3H).

### Example 1-43

### N-(Cyclohexylmethyl)-6-(2,3-dihydro-1,4-benzodioxin-6-yl)-4-oxo-4,5-dihydropyrazolo[1,5-a]-pyrazine-2-carboxamide

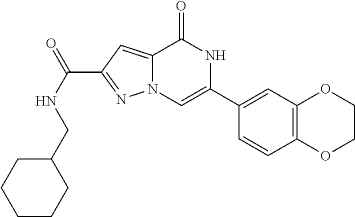 [SMILES: O=C(NCC1CCCCC1)c1cc2c(=O)[nH]c(-c3ccc4c(c3)OCCO4)cn2n1]

The title compound was prepared according to general procedure 1-B starting from 6-(2,3-dihydro-1,4-benzodioxin-6-yl)-4-oxo-4,5-dihydropyrazolo[1,5-a]pyrazine-2-carboxylic acid (Intermediate 102A, 100 mg, 319 μmol), 1-cyclohexylmethanamine (42 μl, 320 μmol), CDI (104 mg, 638 μmol) and DMAP (19.5 mg, 160 μmol) in N-methylpyrrolidone (5.0 ml). After cooling to RT, the mixture was partitioned between brine and ethyl acetate. The layers were separated, and the aqueous layer was extracted with ethyl acetate. The combined organic layers were dried over magnesium sulfate and filtered. The filtrate was concentrated, and the residue was crystallized from acetonitrile to afford the title compound. Yield: 55.4 mg (42% of theory).

LC/MS [Method 7]: Rₜ=0.97 min; MS (ESIpos): m/z=407 [M+H]⁺.

¹H-NMR (400 MHz, DMSO-d₆): δ [ppm]=11.61 (s, 1H), 8.35 (t, 1H), 7.90 (s, 1H), 7.31-7.28 (m, 2H), 7.23 (dd, 1H), 6.96 (d, 1H), 4.32-4.27 (m, 4H), 3.12 (t, 2H), 1.73-1.65 (m, 4H), 1.65-1.52 (m, 2H), 1.25-1.10 (m, 3H), 0.98-0.86 (m, 2H).

### Example 1-44

### N-[1-(4-Fluorophenyl)-2-(pyrrolidin-1-yl)ethyl]-6-(2-naphthyl)-4-oxo-5H-pyrazolo[1,5-a]pyrazine-2-carboxamide

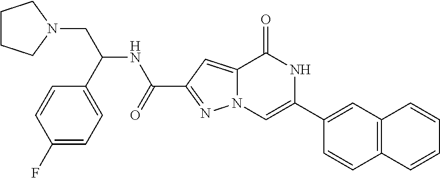 [SMILES: O=C(NC(CN1CCCC1)c1ccc(F)cc1)c1cc2c(=O)[nH]c(-c3ccc4ccccc4c3)cn2n1]

To 6-(naphthalen-2-yl)-4-oxo-4,5-dihydropyrazolo[1,5-a]pyrazine-2-carboxylic acid (Intermediate 3A, 75.0 mg, 98% purity, 240 μmol) in DMF (3.7 ml) was added N,N-diisopropylethylamine (170 μl, 960 μmol) followed by 1-hydroxy-7-azabenzotriazole (200 μl, 0.60 M solution in DMF, 120 μmol) and N-(3-dimethylaminopropyl)-N′-ethylcarbodiimide hydrochloride (50.5 mg, 263 μmol). The mixture was stirred at RT for 1 h. Then, 1-(4-fluorophenyl)-2-(pyrrolidin-1-yl)ethanamine dihydrochloride (73.7 mg, 91% purity, 240 μmol) was added, and the reaction mixture was heated to 50° C. overnight. After cooling to RT, water was added, and the mixture was extracted with ethyl acetate. The combined organic layers were dried over sodium sulfate and filtered, and the filtrate was concentrated under reduced pressure. The residue was triturated with acetonitrile, and the solid was filtered off, washed with a small portion of acetonitrile and dried. The solid material was further purified by preparative HPLC (Method P3) to afford the title compound. Yield: 48.0 mg (40% of theory).

LC/MS [Method 7]: Rₜ=0.75 min; MS (ESIpos): m/z=496 [M+H]⁺.

¹H-NMR (400 MHz, DMSO-d₆): δ [ppm]=11.87 (br. s, 1H), 8.70 (d, 1H), 8.39 (s, 1H), 8.21 (s, 1H), 8.04 (d, 1H), 8.02-7.96 (m, 2H), 7.89 (dd, 1H), 7.63-7.58 (m, 2H), 7.51-7.45 (m, 2H), 7.39 (s, 1H), 7.18-7.13 (m, 2H), 5.18-5.11 (m, 1H), 3.01 (dd, 1H), 2.67-2.61 (m, 1H), 1.69-1.63 (m, 4H).

### Example 1-45

### N-[(1R)-1-(4-Fluorophenyl)ethyl]-3-methyl-6-(naphthalen-2-yl)-4-oxo-4,5-dihydropyrazolo[1,5-a]-pyrazine-2-carboxamide

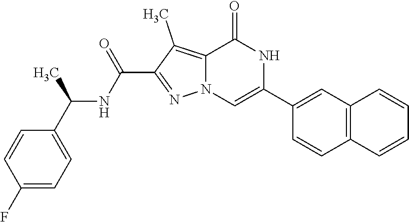 [SMILES: Cc1c(C(=O)N[C@H](C)c2ccc(F)cc2)nn2cc(-c3ccc4ccccc4c3)[nH]c(=O)c12]

To a suspension of 3-methyl-6-(naphthalen-2-yl)-4-oxo-4,5-dihydropyrazolo[1,5-a]pyrazine-2-carboxylic acid (Intermediate 88A, 500 mg, 1.57 mmol) in DMF (20.0 ml) was added N,N-diisopropylethylamine (820 μl, 4.7 mmol) followed by 1-hydroxy-7-azabenzotriazole (1.3 ml, 0.60 M solution in DMF, 780 μmol) and N-(3-dimethylaminopropyl)-N′-ethylcarbodiimide hydrochloride (330 mg, 1.72 mmol). The mixture was stirred at RT for 1 h. Then, (1R)-1-(4-fluorophenyl)ethanamine (218 mg, 1.57 mmol) was added, and stirring at RT was continued for 3 days. The mixture was poured into water, and the precipitate was filtered off. The solid was suspended in ethyl acetate, and the suspension was washed with aqueous sodium bicarbonate solution. The solid material was filtered off and dried to afford the title compound. Yield: 302 mg (42% of theory).

LC/MS [Method 3]: Rₜ=2.15 min; MS (ESIpos): m/z=439 [M+H]⁺.

¹H-NMR (400 MHz, DMSO-d₆): δ [ppm]=11.56 (br. s, 1H), 8.45 (s, 1H), 8.38 (br. s, 1H), 8.03 (s, 1H), 7.99-7.90 (m, 4H), 7.57-7.45 (m, 4H), 7.19-7.12 (m, 2H), 5.20-5.12 (m, 1H), 2.60 (s, 3H), 1.49 (d, 3H).

### Example 1-46

### N-[(1R)-2-(Dimethylamino)-1-phenylethyl]-3,7-dimethyl-6-(naphthalen-2-yl)-4-oxo-4,5-dihydropyrazolo[1,5-a]pyrazine-2-carboxamide formic acid salt

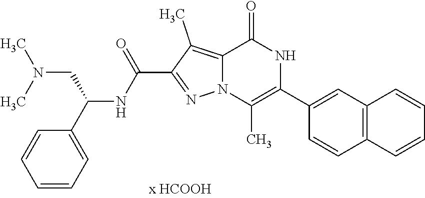 [SMILES: Cc1c(C(=O)N[C@@H](CN(C)C)c2ccccc2)nn2c(C)c(-c3ccc4ccccc4c3)[nH]c(=O)c12]

The title compound was prepared according to general procedure 1-B starting from 3,7-dimethyl-6-(naphthalen-2-yl)-4-oxo-4,5-dihydropyrazolo[1,5-a]pyrazine-2-carboxylic acid (Intermediate 57A, 100 mg, 300 μmol), (1R)—N²,N²-dimethyl-1-phenylethane-1,2-diamine (49.3 mg, 300 μmol), CDI (97.3 mg, 600 μmol) and DMAP (18.3 mg, 150 μmol) in N-methylpyrrolidone (2.9 ml). After cooling to RT, the reaction mixture was directly purified by preparative HPLC (Method P1) to afford the title compound. Yield: 88.5 mg (54% of theory).

LC/MS [Method 3]: Rₜ=1.38 min; MS (ESIpos): m/z=480 [M+H]⁺.

¹H-NMR (400 MHz, DMSO-d₆): δ [ppm]=11.44 (br. s, 1H), 8.43 (d, 1H), 8.15 (s, 1H), 8.10 (s, 1H), 8.08-7.99 (m, 3H), 7.66-7.58 (m, 3H), 7.44 (d, 2H), 7.38-7.31 (m, 2H), 7.29-7.22 (m, 1H), 5.23-5.15 (m, 1H), 2.93-2.85 (m, 1H), 2.64 (s, 3H), 2.44 (s, 3H), 2.26 (s, 6H).

### Example 1-47

### tert.-Butyl 3-(4-fluorophenyl)-3-({[6-(naphthalen-2-yl)-4-oxo-4,5-dihydropyrazolo[1,5-a]pyrazin-2-yl]carbonyl}amino)azetidine-1-carboxylate

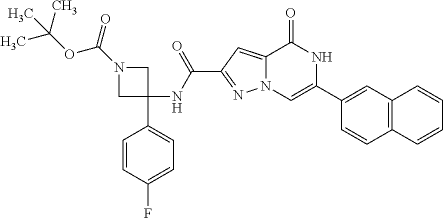 [SMILES: CC(C)(C)OC(=O)N1CC(NC(=O)c2cc3c(=O)[nH]c(-c4ccc5ccccc5c4)cn3n2)(c2ccc(F)cc2)C1]

The title compound was prepared according to general procedure 1-B starting from 6-(naphthalen-2-yl)-4-oxo-4,5-dihydropyrazolo[1,5-a]pyrazine-2-carboxylic acid (Intermediate 3A, 500 mg, 1.64 mmol), tert.-butyl 3-amino-3-(4-fluorophenyl)azetidine-1-carboxylate (Intermediate 103A, 436 mg, 1.64 mmol), CDI (531 mg, 3.28 mmol) and DMAP (100 mg, 819 μmol) in N-methylpyrrolidone (25.0 ml). After cooling to RT, the mixture was poured into water, and the precipitate was filtered off, washed with water and dried under reduced pressure to afford the title compound. Yield: 198 mg (22% of theory).

LC/MS [Method 3]: Rₜ=2.09 min; MS (ESIpos): m/z=576 [M+Na]⁺.

¹H-NMR (400 MHz, DMSO-d₆): δ [ppm]=12.14 (br. s, 1H), 9.63 (s, 1H), 8.38 (s, 1H), 8.15 (s, 1H), 8.05 (d, 1H), 8.02-7.97 (m, 2H), 7.94-7.82 (m, 1H), 7.65-7.57 (m, 2H), 7.56-7.47 (m, 2H), 7.40 (s, 1H), 7.21 (t, 2H), 4.39 (br. d, 2H), 4.24-4.12 (m, 2H), 1.41 (s, 9H).

### Example 1-48

### N-[3-(4-Fluorophenyl)azetidin-3-yl]-6-(naphthalen-2-yl)-4-oxo-4,5-dihydropyrazolo[1,5-a]-pyrazine-2-carboxamide hydrochloride

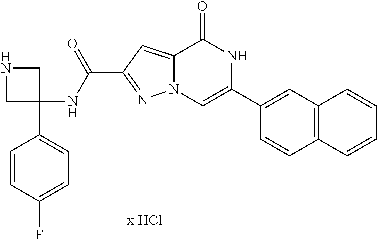 [SMILES: O=C(NC1(c2ccc(F)cc2)CNC1)c1cc2c(=O)[nH]c(-c3ccc4ccccc4c3)cn2n1]

To a solution of ted.-butyl 3-(4-fluorophenyl)-3-({[6-(naphthalen-2-yl)-4-oxo-4,5-dihydropyrazolo-[1,5-a]pyrazin-2-yl]carbonyl}amino)azetidine-1-carboxylate (Example 1-47, 221 mg, 399 μmol) in dichloromethane (10 ml) were added anisole (220 μl, 2.0 mmol) and hydrogen chloride (500 μl, 4.0 M solution in 1,4-dioxane, 2.0 mmol). The mixture was stirred at RT for 2 h. Then, the volatiles were removed under reduced pressure, and the residue was re-dissolved in ethanol and again evaporated to dryness. This procedure was repeated two more times. The residue was triturated with acetonitrile, and the solid was collected by filtration and dried to afford the title compound. Yield: 170 mg (85% of theory).

LC/MS [Method 3]: Rₜ=1.19 min; MS (ESIpos): m/z=454 [M+H]⁺.

¹H-NMR (400 MHz, DMSO-d₆): δ [ppm]=11.94 (s, 1H), 9.86 (s, 1H), 9.57-9.34 (m, 1H), 9.34-9.11 (m, 1H), 8.38 (s, 1H), 8.13 (s, 1H), 8.05 (d, 1H), 8.03-7.96 (m, 2H), 7.88 (dd, 1H), 7.66-7.58 (m, 4H), 7.44 (s, 1H), 7.33-7.21 (m, 2H), 4.64-4.49 (m, 2H), 4.49-4.38 (m, 2H).

### Example 1-49

### N-[3-(4-Fluorophenyl)-1-methylazetidin-3-yl]-6-(naphthalen-2-yl)-4-oxo-4,5-dihydropyrazolo-[1,5-a]pyrazine-2-carboxamide formic acid salt

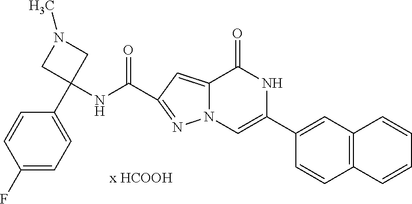 [SMILES: CN1CC(NC(=O)c2cc3c(=O)[nH]c(-c4ccc5ccccc5c4)cn3n2)(c2ccc(F)cc2)C1]

Paraformaldehyde (4.9 mg, 163 μmol) was added to a mixture of N-[3-(4-fluorophenyl)azetidin-3-yl]-6-(naphthalen-2-yl)-4-oxo-4,5-dihydropyrazolo[1,5-a]pyrazine-2-carboxamide hydrochloride (Example 1-48, 80.0 mg, 163 μmol) and acetic acid (93 μl, 1.6 mmol) in dichloromethane (10 ml), and the mixture was stirred at RT for 1 h. Sodium triacetoxyborohydride (51.9 mg, 245 μmol) was added, and stirring at RT was continued overnight. More sodium triacetoxyborohydride (34.6 mg, 163 μmol) was added, and stirring at RT was continued for 5 days. Then, aqueous formaldehyde solution (excess) and sodium triacetoxyborohydride (34.6 mg, 163 μmol) were added, and stirring at RT was further continued overnight. Water was added, and the mixture was neutralized with aqueous sodium bicarbonate solution. The layers were separated, and the aqueous layer was extracted with dichloromethane. The combined organic layers were dried over magnesium sulfate, filtered and concentrated under reduced pressure. The residue was purified by preparative HPLC (Method P1) to afford the title compound. Yield: 31.6 mg (36% of theory).

LC/MS [Method 7]: Rₜ=0.72 min; MS (ESIpos): m/z=468 [M+H]⁺.

¹H-NMR (400 MHz, DMSO-d₆): δ [ppm]=11.86 (br. s, 1H), 9.45 (s, 1H), 8.38 (s, 1H), 8.20-8.17 (m, 1H), 8.15 (s, 1H), 8.05 (d, 1H), 8.02-7.96 (m, 2H), 7.88 (dd, 1H), 7.67-7.57 (m, 4H), 7.38 (s, 1H), 7.21-7.12 (m, 2H), 3.72 (br. d, 2H), 3.48 (br. d, 2H), 2.33 (s, 3H).

### Example 1-50

### Methyl 3-(4-fluorophenyl)-3-({[6-(naphthalen-2-yl)-4-oxo-4,5-dihydropyrazolo[1,5-a]pyrazin-2-yl]-carbonyl}amino)azetidine-1-carboxylate

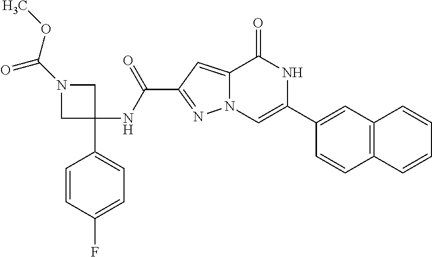 [SMILES: COC(=O)N1CC(NC(=O)c2cc3c(=O)[nH]c(-c4ccc5ccccc5c4)cn3n2)(c2ccc(F)cc2)C1]

To a solution of N-[3-(4-fluorophenyl)azetidin-3-yl]-6-(naphthalen-2-yl)-4-oxo-4,5-dihydropyrazolo-[1,5-a]pyrazine-2-carboxamide hydrochloride (Example 1-48, 100 mg, 88% purity, 180 μmol) in dichloromethane (10 ml) was added N,N-diisopropylethylamine (130 μl, 720 μmol) followed by methyl carbonochloridate (15 μl, 200 μmol). The mixture was stirred at RT overnight. The volatiles were removed under reduced pressure, and the residue was re-dissolved in methanol (5.0 ml). DMAP (11.0 mg, 89.8 μmol) and 1-aminopropane (9 μl, 720 μmol) were added, and the mixture was stirred at RT for 1 h and then concentrated. The residue was partitioned between water and ethyl acetate, the layers were separated, and the aqueous layer was extracted with ethyl acetate. The combined organic layers were dried over magnesium sulfate and filtered, and the filtrate was concentrated. The residue was purified by preparative HPLC (Method P10) to afford the title compound. Yield: 8.0 mg (9% of theory).

LC/MS [Method 3]: Rₜ=1.78 min; MS (ESIpos): m/z=512 [M+H]⁺.

¹H-NMR (400 MHz, DMSO-d₆): δ [ppm]=11.58 (br. s, 1H), 9.65 (s, 1H), 8.38 (s, 1H), 8.15 (s, 1H), 8.05 (d, 1H), 8.02-7.97 (m, 2H), 7.88 (dd, 1H), 7.64-7.56 (m, 2H), 7.56-7.49 (m, 2H), 7.40 (s, 1H), 7.28-7.14 (m, 2H), 4.46 (br. d, 2H), 4.24 (br. d, 2H), 3.61 (s, 3H).

### Example 1-51

### 6-(3,4-Dihydro-1H-isochromen-7-yl)-N-[(1R)-1-(6-methoxypyridin-3-yl)ethyl]-4-oxo-4,5-dihydropyrazolo[1,5-a]pyrazine-2-carboxamide

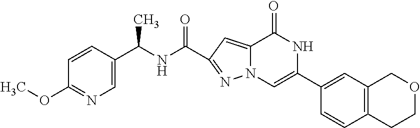 [SMILES: COc1ccc([C@@H](C)NC(=O)c2cc3c(=O)[nH]c(-c4ccc5c(c4)COCC5)cn3n2)cn1]

The title compound was prepared according to general procedure 1-B starting from 6-(3,4-dihydro-1H-isochromen-7-yl)-4-oxo-4,5-dihydropyrazolo[1,5-a]pyrazine-2-carboxylic acid (Intermediate 66A, 100 mg, 321 μmol), (1R)-1-(6-methoxypyridin-3-yl)ethanamine (Intermediate 49A, 53.8 mg, 353 μmol), CDI (57.3 mg, 353 μmol) and DMAP (19.6 mg, 161 μmol) in N-methylpyrrolidone (2.7 ml). After cooling to RT, the mixture was diluted with DMSO (5.0 ml) and directly purified by preparative HPLC (Method P11). The product-containing fractions were combined and lyophilized. The residue was dissolved in dichloromethane and further purified by column chromatography on silica gel (eluent: gradient of methanol in dichloromethane) to afford the title compound. Yield: 42.3 mg (30% of theory).

LC/MS [Method 3]: Rₜ=1.48 min; MS (ESIpos): m/z=446 [M+H]⁺.

¹H-NMR (400 MHz, DMSO-d₆): δ [ppm]=11.69 (s, 1H), 8.82 (d, 1H), 8.19 (d, 1H), 7.95 (s, 1H), 7.79 (dd, 1H), 7.53 (dd, 1H), 7.44 (s, 1H), 7.36 (s, 1H), 7.26 (d, 1H), 6.80 (d, 1H), 5.19-5.11 (m, 1H), 4.73 (s, 2H), 3.94-3.88 (m, 2H), 3.82 (s, 3H), 2.88-2.79 (m, 2H), 1.51 (d, 3H).

### Example 1-52

### N-[(1R)-1-(4-Fluorophenyl)-3-hydroxpropyl]-6-(naphthalen-2-yl)-4-oxo-4,5-dihydropyrazolo-[1,5-a]pyrazine-2-carboxamide

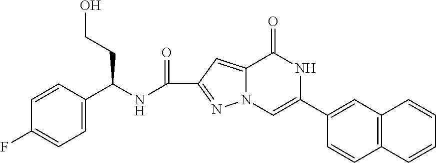 [SMILES: O=C(N[C@H](CCO)c1ccc(F)cc1)c1cc2c(=O)[nH]c(-c3ccc4ccccc4c3)cn2n1]

The title compound was prepared according to general procedure 1-B starting from 6-(naphthalen-2-yl)-4-oxo-4,5-dihydropyrazolo[1,5-a]pyrazine-2-carboxylic acid (Intermediate 3A, 90.0 mg, 295 μmol), (3R)-3-amino-3-(4-fluorophenyl)propan-1-ol (54.9 mg, 324 μmol), CDI (52.6 mg, 324 μmol) and DMAP (18.0 mg, 147 μmol) in N-methylpyrrolidone (2.5 ml). After cooling to RT, the reaction mixture was directly purified by preparative HPLC (Method P2) to afford the title compound. Yield: 59.2 mg (44% of theory).

LC/MS [Method 3]: Rₜ=1.69 min; MS (ESIpos): m/z=457 [M+H]⁺.

¹H-NMR (400 MHz, DMSO-d₆): δ [ppm]=11.87 (s, 1H), 8.91 (d, 1H), 8.40-8.37 (m, 1H), 8.18 (s, 1H), 8.04 (d, 1H), 8.02-7.96 (m, 2H), 7.88 (dd, 1H), 7.63-7.58 (m, 2H), 7.48-7.41 (m, 2H), 7.38 (s, 1H), 7.19-7.13 (m, 2H), 5.23-5.16 (m, 1H), 4.74-4.48 (m, 1H), 3.49-3.39 (m, 2H), 2.13-2.04 (m, 1H), 1.98-1.89 (m, 1H).

### Example 1-53

### N-[(1R)-1-(4-Fluorophenyl)ethyl]-6-(naphthalen-2-yl)-4-oxo-3-(trifluoromethyl)-4,5-dihydropyrazolo[1,5-a]pyrazine-2-carboxamide

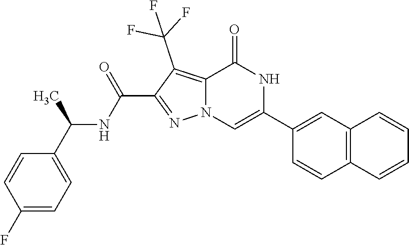 [SMILES: C[C@@H](NC(=O)c1nn2cc(-c3ccc4ccccc4c3)[nH]c(=O)c2c1C(F)(F)F)c1ccc(F)cc1]

The title compound was prepared according to general procedure 1-B starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), (1R)-1-(4-fluorophenyl)ethanamine (36 μl, 270 μmol), CDI (86.9 mg, 536 μmol) and DMAP (16.4 mg, 134 μmol) in N-methylpyrrolidone (4.0 ml). After cooling to RT, the mixture was poured into water and extracted with dichloromethane. The combined organic layers were washed with brine, dried over magnesium sulfate and filtered, and the filtrate was concentrated under reduced pressure. The residue was purified by preparative HPLC (Method P2) to afford the title compound. Yield: 64.6 mg (49% of theory).

LC/MS [Method 27]: Rₜ=1.40 min; MS (ESIpos): m/z=495 [M+H]⁺.

¹H-NMR (400 MHz, DMSO-d₆): δ [ppm]=12.24 (s, 1H), 9.27 (d, 1H), 8.40 (d, 2H), 8.05 (d, 1H), 8.03-7.97 (m, 2H), 7.89 (dd, 1H), 7.65-7.58 (m, 2H), 7.47-7.40 (m, 2H), 7.24-7.13 (m, 2H), 5.20-5.12 (m, 1H), 1.45 (d, 3H).

### Example 1-54

### 6-(3,4-Dimethylphenyl)-N-[(1S)-2-hydroxy-2-methyl-1-phenylpropyl]-4-oxo-4,5-dihydropyrazolo-[1,5-a]pyrazine-2-carboxamide

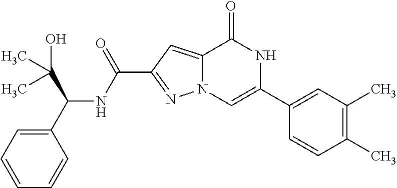 [SMILES: Cc1ccc(-c2cn3nc(C(=O)N[C@@H](c4ccccc4)C(C)(C)O)cc3c(=O)[nH]2)cc1C]

To a suspension of 6-(3,4-dimethylphenyl)-4-oxo-4,5-dihydropyrazolo[1,5-a]pyrazine-2-carboxylic acid (Intermediate 23A, 100 mg, 353 μmol) in DMF (3.0 ml) were added N,N-diisopropylethylamine (180 μl, 1.1 mmol), 1-hydroxy-7-azabenzotriazole (350 μl, 0.50 M solution in DMF, 180 μmol) and N-(3-dimethylaminopropyl)-N′-ethylcarbodiimide hydrochloride (74.4 mg, 388 μmol). The mixture was stirred at RT for 1 h. Then, DMAP (4.31 mg, 35.3 μmol) and (1S)-1-amino-2-methyl-1-phenylpropan-2-ol (Intermediate 4A, 64.2 mg, 388 μmol) were added, and the mixture was stirred at 50° C. overnight. After cooling to RT, the mixture was poured into water, and the precipitate was filtered off, washed with water and dried. The solid was dissolved in dichloromethane and purified by column chromatography on basic silica gel (eluent: gradient of methanol in dichloromethane) to afford the title compound. Yield: 17.2 mg (11% of theory).

LC/MS [Method 3]: Rₜ=1.81 min; MS (ESIpos): m/z=431 [M+H]⁺.

¹H-NMR (400 MHz, DMSO-d₆): δ [ppm]=11.67 (br. s, 1H), 8.28 (d, 1H), 8.15 (s, 1H), 7.61-7.58 (m, 1H), 7.53-7.47 (m, 1H), 7.40 (d, 2H), 7.34-7.23 (m, 5H), 4.95 (s, 1H), 4.84 (d, 1H), 2.29 (s, 3H), 2.28 (s, 3H), 1.26 (s, 3H), 0.98 (s, 3H).

### Example 1-55

### N-[(1R)-1-(4-Fluorophenyl)-3-hydroxopyl]-3-(methoxymethyl)-6-(naphthalen-2-yl)-4-oxo-4,5-dihydropyrazolo[1,5-a]pyrazine-2-carboxamide

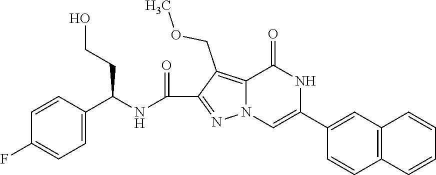 [SMILES: COCc1c(C(=O)N[C@H](CCO)c2ccc(F)cc2)nn2cc(-c3ccc4ccccc4c3)[nH]c(=O)c12]

The title compound was prepared according to general procedure 1-B starting from 3-(methoxymethyl)-6-(naphthalen-2-yl)-4-oxo-4,5-dihydropyrazolo[1,5-a]pyrazine-2-carboxylic acid (Intermediate 42A, 100 mg, 286 μmol), (3R)-3-amino-3-(4-fluorophenyl)propan-1-ol (53.3 mg, 315 μmol), CDI (51.1 mg, 315 μmol) and DMAP (17.5 mg, 143 μmol) in N-methylpyrrolidone (2.1 ml). After cooling to RT, the reaction mixture was directly purified by preparative HPLC (Method P2) to afford the title compound. Yield: 60.0 mg (42% of theory).

LC/MS [Method 3]: Rₜ=1.76 min; MS (ESIpos): m/z=501 [M+H]⁺.

¹H-NMR (400 MHz, DMSO-d₆): δ [ppm]=11.83 (s, 1H), 8.85 (d, 1H), 8.38 (s, 1H), 8.17 (d, 1H), 8.03 (d, 1H), 8.01-7.95 (m, 2H), 7.89 (dd, 1H), 7.63-7.56 (m, 2H), 7.48-7.40 (m, 2H), 7.20-7.13 (m, 2H), 5.23-5.15 (m, 1H), 4.92 (s, 2H), 3.51-3.42 (m, 2H), 3.23 (s, 3H), 2.10-1.98 (m, 1H), 1.98-1.88 (m, 1H).

### Example 1-56

N-[(1R)-1-(4-Fluorophenyl)-3-hydroxypropyl]-6-(5-methylquinolin-3-yl)-4-oxo-3-(propan-2-yl)-4,5-dihydropyrazolo[1,5-a]pyrazine-2-carboxamide

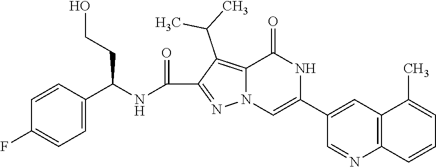 [SMILES: Cc1cccc2ncc(-c3cn4nc(C(=O)N[C@H](CCO)c5ccc(F)cc5)c(C(C)C)c4c(=O)[nH]3)cc12]

The title compound was prepared according to general procedure 1-A starting from 6-(5-methyl-quinolin-3-yl)-4-oxo-3-(propan-2-yl)-4,5-dihydropyrazolo[1,5-a]pyrazine-2-carboxylic acid (Intermediate 77A, 100 mg, 276 μmol), (3R)-3-amino-3-(4-fluorophenyl)propan-1-ol (51.4 mg, 304 μmol), N,N-diisopropylethylamine (140 μl, 830 μmol) and HATU (126 mg, 331 μmol) in N-methylpyrrolidone (4.5 ml, 46 mmol). After cooling to RT, the reaction mixture was filtered, and the filtrate was purified by preparative HPLC (Method P7) to afford the title compound. Yield: 61.0 mg (42% of theory).

LC/MS [Method 3]: Rₜ=1.82 min; MS (ESIpos): m/z=514 [M+H]⁺.

¹H-NMR (400 MHz, DMSO-d₆): δ [ppm]=11.87 (br. s, 1H), 9.26 (d, 1H), 8.86-8.81 (m, 2H), 8.38 (s, 1H), 7.90 (d, 1H), 7.73-7.68 (m, 1H), 7.52 (d, 1H), 7.49-7.42 (m, 2H), 7.20-7.14 (m, 2H), 5.22-5.16 (m, 1H), 4.62 (t, 1H), 4.11-4.02 (m, 1H), 3.48-3.37 (m, 2H), 2.77 (s, 3H), 2.09-2.00 (m, 1H), 1.96-1.87 (m, 1H), 1.34 (d, 3H), 1.31 (d, 3H).

### Example 1-57

### tert.-Butyl 3-(4-methoxyphenyl)-3-({[6-(naphthalen-2-yl)-4-oxo-4,5-dihydropyrazolo[1,5-a]pyrazin-2-yl]carbonyl}amino)azetidine-1-carboxylate

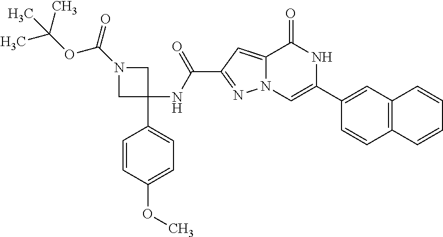 [SMILES: COc1ccc(C2(NC(=O)c3cc4c(=O)[nH]c(-c5ccc6ccccc6c5)cn4n3)CN(C(=O)OC(C)(C)C)C2)cc1]

The title compound was prepared according to general procedure 1-B starting from 6-(naphthalen-2-yl)-4-oxo-4,5-dihydropyrazolo[1,5-a]pyrazine-2-carboxylic acid (Intermediate 3A, 200 mg, 655 μmol), tert.-butyl 3-amino-3-(4-methoxyphenyl)azetidine-1-carboxylate (Intermediate 106A, 182 mg, 655 μmol), CDI (212 mg, 1.31 mmol) and DMAP (40.0 mg, 328 μmol) in N-methylpyrrolidone (10.0 ml). After cooling to RT, the reaction mixture was directly purified by preparative HPLC (Method P1). The product-containing fractions were combined and evaporated. The residue was triturated in acetonitrile, and the solid was collected by filtration, washed with acetonitrile and dried to afford the title compound. Yield: 160 mg (43% of theory).

LC/MS [Method 3]: Rₜ=2.06 min; MS (ESIpos): m/z=566 [M+H]⁺.

¹H-NMR (400 MHz, DMSO-d₆): δ [ppm]=11.90 (br. s, 1H), 9.53 (s, 1H), 8.38 (s, 1H), 8.16 (s, 1H), 8.05 (d, 1H), 8.02-7.95 (m, 2H), 7.88 (d, 1H), 7.65-7.56 (m, 2H), 7.44-7.35 (m, 3H), 6.94 (d, 2H), 4.38 (d, 2H), 4.24-4.05 (m, 2H), 3.75 (s, 3H), 1.41 (s, 9H).

### Example 1-58

### 3-Cyano-6-(3,4-dimethylphenyl)-N-[(1R)-1-(4-fluorophenyl)-3-hydroxpropyl]-4-oxo-4,5-dihydropyrazolo[1,5-a]pyrazine-2-carboxamide

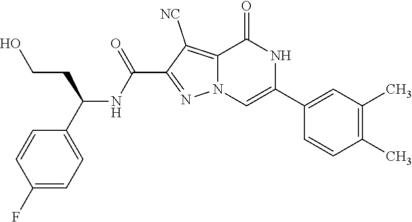 [SMILES: Cc1ccc(-c2cn3nc(C(=O)N[C@H](CCO)c4ccc(F)cc4)c(C#N)c3c(=O)[nH]2)cc1C]

The title compound was prepared according to general procedure 1-B starting from 3-cyano-6-(3,4-dimethylphenyl)-4-oxo-4,5-dihydropyrazolo[1,5-a]pyrazine-2-carboxylic acid (Intermediate 108A, 100 mg, 324 μmol), (3R)-3-amino-3-(4-fluorophenyl)propan-1-ol (60.4 mg, 357 μmol), CDI (57.9 mg, 357 μmol) and DMAP (19.8 mg, 162 μmol) in N-methylpyrrolidone (2.7 ml). After cooling to RT, the reaction mixture was diluted with DMSO (5.0 ml), and the solution was directly purified by preparative HPLC (Method P5) to afford the title compound. Yield: 25.0 mg (16% of theory).

LC/MS [Method 11]: Rₜ=2.83 min; MS (ESIpos): m/z=460 [M+H]⁺.

¹H-NMR (400 MHz, DMSO-d₆): δ [ppm]=12.19 (br. s, 1H), 9.18 (d, 1H), 8.05 (s, 1H), 7.57 (s, 1H), 7.51-7.43 (m, 3H), 7.28 (d, 1H), 7.20-7.13 (m, 2H), 5.22-5.16 (m, 1H), 4.65 (t, 1H), 3.47-3.33 (m, 2H), 2.29 (s, 3H), 2.28 (s, 3H), 2.13-2.04 (m, 1H), 1.97-1.88 (m, 1H).

### Example 1-59

### N-[3-(4-Methoxyphenyl)-1-methylazetidin-3-yl]-6-(naphthalen-2-yl)-4-oxo-4,5-dihydropyrazolo-[1,5-a]pyrazine-2-carboxamide formic acid salt

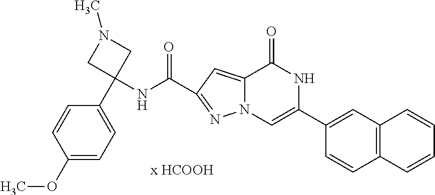 [SMILES: COc1ccc(C2(NC(=O)c3cc4c(=O)[nH]c(-c5ccc6ccccc6c5)cn4n3)CN(C)C2)cc1]

The title compound was prepared according to general procedure 1-B starting from 6-(naphthalen-2-yl)-4-oxo-4,5-dihydropyrazolo[1,5-a]pyrazine-2-carboxylic acid (Intermediate 3A, 200 mg, 655 μmol), 3-(4-methoxphenyl)-1-methylazetidin-3-amine (Intermediate 109A, 126 mg, 655 μmol), CDI (212 mg, 1.31 mmol) and DMAP (40.0 mg, 328 μmol) in N-methylpyrrolidone (10.0 ml). After cooling to RT, the reaction mixture was directly purified by preparative HPLC (Method P1) to afford the title compound. Yield: 207 mg (60% of theory).

LC/MS [Method 3]: Rₜ=1.19 min; MS (ESIpos): m/z=480 [M+H]⁺.

¹H-NMR (400 MHz, DMSO-d₆): δ [ppm]=11.52 (br. s, 1H), 9.38 (s, 1H), 8.38 (s, 1H), 8.17 (s, 1H), 8.15 (s, 1H), 8.05 (d, 1H), 8.02-7.97 (m, 2H), 7.88 (d, 1H), 7.64-7.58 (m, 2H), 7.53-7.48 (m, 2H), 7.38 (s, 1H), 6.93-6.88 (m, 2H), 3.83-3.75 (m, 2H), 3.74 (s, 3H), 3.65-3.57 (m, 2H), 2.39 (s, 2H), 2.35-2.35 (m, 1H).

### Example 1-60

### 3-Cyano-N-[(1S)-2-methoxy-1-phenylethyl]-6-(naphthalen-2-yl)-4-oxo-4,5-dihydropyrazolo[1,5-a]-pyrazine-2-carboxamide

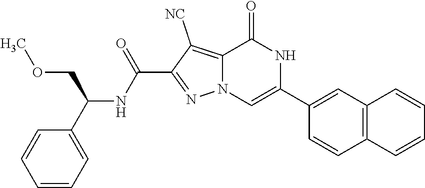 [SMILES: COC[C@@H](NC(=O)c1nn2cc(-c3ccc4ccccc4c3)[nH]c(=O)c2c1C#N)c1ccccc1]

The title compound was prepared according to general procedure 1-B starting from 3-cyano-6-(naphthalen-2-yl)-4-oxo-4,5-dihydropyrazolo[1,5-a]pyrazine-2-carboxylic acid (Intermediate 111A, 100 mg, 303 μmol), (1S)-2-methoxy-1-phenylethanamine (50 μl, 330 μmol), CDI (54.0 mg, 333 μmol) and DMAP (18.5 mg, 151 μmol) in N-methylpyrrolidone (2.5 ml). After cooling to RT, the reaction mixture was diluted with 1.0 M aqueous hydrochloric acid (1.0 ml) and DMSO (5.0 ml), and the resulting solution was directly purified by preparative HPLC (Method P5) to afford the title compound. Yield: 44.3 mg (32% of theory).

LC/MS [Method 7]: Rₜ=1.02 min; MS (ESIpos): m/z=464 [M+H]⁺.

¹H-NMR (400 MHz, DMSO-d₆): δ [ppm]=12.41 (s, 1H), 9.08 (d, 1H), 8.40 (s, 1H), 8.30 (s, 1H), 8.06 (d, 1H), 8.03-7.98 (m, 2H), 7.89 (dd, 1H), 7.65-7.59 (m, 2H), 7.46 (d, 2H), 7.39-7.32 (m, 2H), 7.32-7.26 (m, 1H), 5.30-5.23 (m, 1H), 3.83-3.77 (m, 1H), 3.70-3.55 (m, 1H).

### Example 1-61

### N-[1-(4-Fluorophenyl)-2-hydroxyethyl]-6-(2-naphthyl)-4-oxo-5H-pyrazolo[1 ,5-a]pyrazine-2-carboxamide

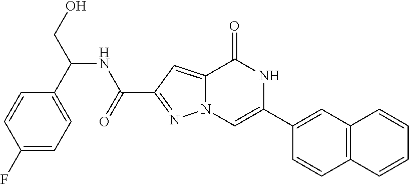 [SMILES: O=C(NC(CO)c1ccc(F)cc1)c1cc2c(=O)[nH]c(-c3ccc4ccccc4c3)cn2n1]

To a solution of 6-(naphthalen-2-yl)-4-oxo-4,5-dihydropyrazolo[1,5-a]pyrazine-2-carboxylic acid (Intermediate 3A, 100 mg, 328 μmol) and N,N-diisopropylethylamine (170 μl, 980 μmol) in DMF (5.0 ml) were added 1-(3-dimethylaminopropyl)-3-ethylcarbodiimide hydrochloride (69.1 mg, 360 μmol) and 1-hydroxy-7-azabenzotriazole (22.3 mg, 164 μmol). The mixture was stirred at RT for 1 h. Then, 2-amino-2-(4-fluorophenyl)ethanol (50.8 mg, 328 μmol) was added, and stirring was continued overnight at RT, followed by heating to 50° C. for 5 h. After cooling to RT, the mixture was poured into water, and the precipitate was collected by filtration, washed with water and dried to afford the title compound. Yield: 66.1 mg (44% of theory).

LC/MS [Method 7]: Rₜ=0.90 min; MS (ESIpos): m/z=443 [M+H]⁺.

¹H-NMR (400 MHz, DMSO-d₆): δ [ppm]=11.88 (br. s, 1H), 8.64 (d, 1H), 8.39 (s, 1H), 8.23 (s, 1H), 8.04 (d, 1H), 8.01-7.96 (m, 2H), 7.89 (dd, 1H), 7.64-7.57 (m, 2H), 7.48-7.39 (m, 3H), 7.20-7.12 (m, 2H), 5.11-5.01 (m, 2H), 3.79-3.67 (m, 2H).

### Example 1-62

### N-[1-(4-Fluorophenyl)-3-hydroxpropyl]-4-oxo-6-(7-quinolyl)-5H-pyrazolo[1,5-a]pyrazine-2-carboxamide

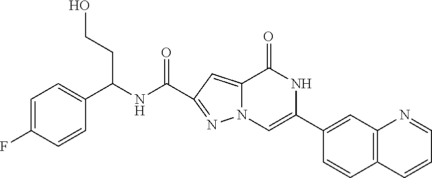 [SMILES: O=C(NC(CCO)c1ccc(F)cc1)c1cc2c(=O)[nH]c(-c3ccc4cccnc4c3)cn2n1]

Oxalyl chloride (280 μl, 3.3 mmol) was added to a suspension of 4-oxo-6-(quinolin-7-yl)-4,5-dihydropyrazolo[1,5-a]pyrazine-2-carboxylic acid (Intermediate 117A, 200 mg, 653 μmol) in dichloromethane (5.5 ml), followed by one drop of DMF. After stirring at RT for 1 h, the volatiles were removed under reduced pressure, and the residue was slowly added to a solution of 3-amino-3-(4-fluorophenyl)propan-1-ol (110 mg, 653 μmol) and N,N-diisopropylethylamine (340 μl, 2.0 mmol) in dichloromethane (5.0 ml). The mixture was stirred at RT overnight. Dichloromethane and water were added, the layers were separated, and the aqueous layer was extracted with dichloromethane. The combined organic layers were dried over magnesium sulfate, filtered and concentrated. The residue was purified by preparative HPLC (Method P9) to afford the title compound. Yield: 2.1 mg (1% of theory).

LC/MS [Method 7]: Rₜ=0.70 min; MS (ESIpos): m/z=458 [M+H]⁺.

¹H-NMR (500 MHz, DMSO-d₆): δ [ppm]=11.63 (br. s, 1H), 8.99 (dd, 1H), 8.91 (d, 1H), 8.45 (d, 1H), 8.43 (dd, 1H), 8.25 (s, 1H), 8.11 (d, 1H), 7.99 (dd, 1H), 7.61 (dd, 1H), 7.48-7.42 (m, 2H), 7.39 (d, 1H), 7.19-7.13 (m, 2H), 5.23-5.17 (m, 1H), 4.63 (t, 1H), 3.49-3.37 (m, 2H), 2.12-2.04 (m, 1H), 1.98-1.89 (m, 1H).

### Example 1-63

### 3-Chloro-6-(3,4-dimethylphenyl)-N-[(1R)-1-(4-fluorophenyl)-3-hydroxypropyl]-4-oxo-4,5-dihydropyrazolo[1,5-a]pyrazine-2-carboxamide

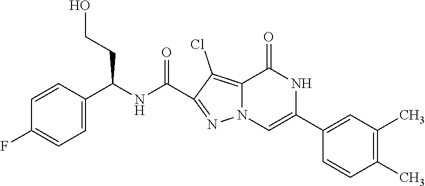 [SMILES: Cc1ccc(-c2cn3nc(C(=O)N[C@H](CCO)c4ccc(F)cc4)c(Cl)c3c(=O)[nH]2)cc1C]

The title compound was prepared according to general procedure 1-A starting from 3-chloro-6-(3,4-dimethylphenyl)-4-oxo-4,5-dihydropyrazolo[1,5-a]pyrazine-2-carboxylic acid (Intermediate 121A, 100 mg, 315 μmol), (3R)-3-amino-3-(4-fluorophenyl)propan-1-ol (63.9 mg, 378 μmol), N,N-diisopropylethylamine (160 μl, 940 μmol) and HATU (144 mg, 378 μmol) in DMF (2.7 ml). After cooling to RT, the mixture was poured into water (100 ml) and extracted with dichloromethane. The combined organic layers were washed with brine, dried over magnesium sulfate, filtered and concentrated. The residue was purified by column chromatography on basic silica gel (eluent: gradient of methanol in dichloromethane). Yield: 14.9 mg (10% of theory).

LC/MS [Method 3]: Rₜ=1.72 min; MS (ESIpos): m/z=469 [M+H]⁺.

¹H-NMR (500 MHz, DMSO-d₆): δ [ppm]=11.57 (br. s, 1H), 8.91 (d, 1H), 7.94 (s, 1H), 7.56 (s, 1H), 7.49-7.40 (m, 3H), 7.25 (d, 1H), 7.19-7.13 (m, 2H), 5.19-5.13 (m, 1H), 4.62 (t, 1H), 3.49-3.34 (m, 2H), 2.28 (s, 3H), 2.27 (s, 3H), 2.08-2.00 (m, 1H), 1.94-1.87 (m, 1H).

### Example 1-64

### tert.-Butyl 3-({[6-(3,4-dimethylphenyl)-4-oxo-4,5-dihydropyrazolo[1,5-a]pyrazin-2-yl]carbonyl}-amino)-3-(4-methoxyphenyl)azetidine-1-carboxylate

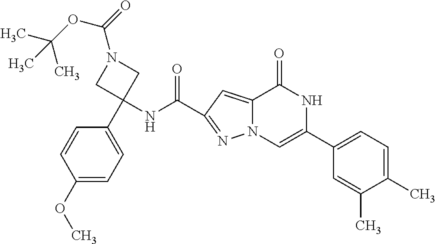 [SMILES: COc1ccc(C2(NC(=O)c3cc4c(=O)[nH]c(-c5ccc(C)c(C)c5)cn4n3)CN(C(=O)OC(C)(C)C)C2)cc1]

N,N-Diisopropylethylamine (1.1 ml, 6.4 mmol) was added to a suspension of 6-(3,4-dimethyl-phenyl)-4-oxo-4,5-dihydropyrazolo[1,5-a]pyrazine-2-carboxylic acid (Intermediate 23A, 600 mg, 2.12 mmol) in DMF (20.0 ml), followed by 1-(3-dimethylaminopropyl)-3-ethylcarbodiimide hydrochloride (447 mg, 2.33 mmol) and 1-hydroxy-7-azabenzotriazole (1.1 ml, 1.1 mmol, 1.0 M solution in DMF). The resulting mixture was stirred at RT for 1 h before tert.-butyl 3-amino-3-(4-methoxyphenyl)azetidine-1-carboxylate (Intermediate 106A, 590 mg, 2.12 mmol) was added. After the addition, stirring was continued at RT overnight. Then, DMAP (15 mg, 123 μmol) was added, and the mixture was heated to 50° C. for 3 h. After cooling to RT, the mixture was poured into water (150 ml), and the precipitate was collected by filtration and washed with water. The solid was dissolved in a mixture of acetonitrile and water and lyophilized. Yield: 670 mg (57% of theory, 99% purity).

LC/MS [Method 3]: Rₜ=2.04 min; MS (ESIpos): m/z=544 [M+H]⁺.

¹H-NMR (400 MHz, DMSO-d₆): δ [ppm]=11.68 (br. s, 1H), 9.50 (s, 1H), 7.93 (s, 1H), 7.56 (s, 1H), 7.47 (d, 1H), 7.41-7.33 (m, 3H), 7.26 (d, 1H), 6.93 (d, 2H), 4.37 (br. d, 2H), 4.24-3.98 (m, 2H), 3.74 (s, 3H), 2.29 (s, 3H), 2.28 (s, 3H), 1.40 (s, 9H).

### Example 1-65

### 6-(3,4-Dimethylphenyl)-N-[3-(4-methoxyphenyl)azetidin-3-yl]-4-oxo-4,5-dihydropyrazolo[1,5-a]-pyrazine-2-carboxamide hydrochloride

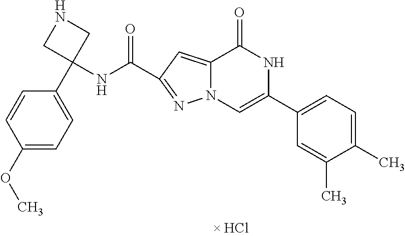 [SMILES: COc1ccc(C2(NC(=O)c3cc4c(=O)[nH]c(-c5ccc(C)c(C)c5)cn4n3)CNC2)cc1]

A solution of tert.-butyl 3-({[6-(3,4-dimethylphenyl)-4-oxo-4,5-dihydropyrazolo[1,5-a]pyrazin-2-yl]-carbonyl}amino)-3-(4-methoxyphenyl)azetidine-1-carboxylate (Example 1-64, 640 mg, 1.18 mmol) in dichloromethane (100 ml) was treated with hydrogen chloride (1.5 ml, 4.0 M solution in 1,4-dioxane, 5.9 mmol) and anisole (640 μl, 5.9 mmol) at RT for 4 h. The mixture was then concentrated under reduced pressure, and the residue was re-dissolved in ethanol and again concentrated to dryness. This procedure was repeated three more times. Finally, the residue was dried to afford the crude title compound which was used for the next steps without further purification. Yield: 580 mg (80% of theory, 78% purity).

LC/MS [Method 7]: Rₜ=0.65 min; MS (ESIpos): m/z=444 [M+H]⁺.

### Example 1-66

### Methyl 3-({[6-(3,4-dimethylphenyl)-4-oxo-4,5-dihydropyrazolo[1,5-a]pyrazin-2-yl]carbonyl}amino)-3-(4-methoxyphenyl)azetidine-1-carboxylate

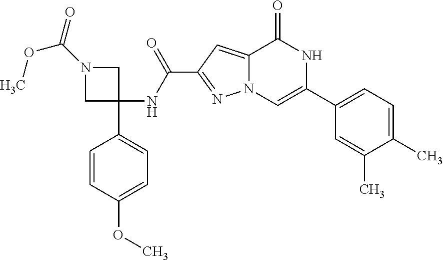

To a solution of 6-(3,4-dimethylphenyl)-N-[3-(4-methoxyphenyl)azetidin-3-yl]-4-oxo-4,5-dihydropyrazolo[1,5-a]pyrazine-2-carboxamide hydrochloride (Example 1-65, 80.0 mg, 167 μmol) and N,N-diisopropylethylamine (87 μl, 500 μmol) in dichloromethane (10.0 ml) was added methyl carbonochloridate (13 μl, 170 μmol), and the mixture was stirred at RT for 2 h. Then, water was added, and the mixture was extracted with dichloromethane. The combined organic layers were dried over magnesium sulfate, filtered and concentrated. The residue was purified by preparative HPLC (Method P1) to afford the title compound. Yield: 40.2 mg (48% of theory).

LC/MS [Method 3]: Rₜ=1.76 min; MS (ESIpos): m/z=502 [M+H]⁺.

¹H-NMR (400 MHz, DMSO-d₆): δ [ppm]=11.68 (s, 1H), 9.51 (s, 1H), 7.93 (s, 1H), 7.55 (s, 1H), 7.47 (dd, 1H), 7.42-7.34 (m, 3H), 7.26 (d, 1H), 6.93 (d, 2H), 4.44 (br. d, 2H), 4.21 (br. d, 2H), 3.74 (s, 3H), 3.60 (s, 3H), 2.29 (s, 3H), 2.28 (s, 3H).

### Example 1-67

### 6-(3,4-Dimethylphenyl)-N-[(1S)-1-(4-fluorophenyl)-2-hydroxy-2-methylpropyl]-4-oxo-3-(trifluoro-methyl)-4,5-dihydropyrazolo[1,5-a]pyrazine-2-carboxamide

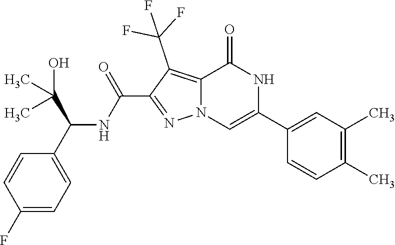 [SMILES: Cc1ccc(-c2cn3nc(C(=O)N[C@@H](c4ccc(F)cc4)C(C)(C)O)c(C(F)(F)F)c3c(=O)[nH]2)cc1C]

A solution of 6-(3,4-dimethylphenyl)-4-oxo-3-(trifluoromethyl)-4,5-dihydropyrazolo[1,5-a]pyrazine-2-carboxylic acid (Intermediate 125A, 83.1 mg, 237 μmol), HATU (108 mg, 284 μmol) and N,N-diisopropylethylamine (120 μl, 710 μmol) in DMF (2.1 ml) was stirred at RT for 10 min before (1S)-1-amino-1-(4-fluorophenyl)-2-methylpropan-2-ol (Intermediate 36A, 52.0 mg, 284 μmol) was added. After the addition, stirring was continued at RT overnight. Then, water (10 ml) was added, and the precipitate was collected by filtration and washed with water. The crude product was purified by column chromatography on silica gel (eluent: gradient of methanol in dichloromethane). Yield: 19.0 mg (15% of theory).

LC/MS [Method 7]: Rₜ=1.05 min; MS (ESIpos): m/z=517 [M+H]⁺.

¹H-NMR (500 MHz, DMSO-d₆): δ [ppm]=12.06 (br. s, 1H), 8.88 (d, 1H), 8.20 (s, 1H), 7.61 (s, 1H), 7.52 (dd, 1H), 7.43-7.42 (m, 1H), 7.46-7.41 (m, 1H), 7.25 (d, 1H), 7.18-7.10 (m, 2H), 4.93 (d, 1H), 4.65 (br. s, 1H), 2.29 (s, 3H), 2.27 (s, 3H), 1.23-1.16 (m, 3H), 1.04 (s, 3H).

### Example 1-68

### N-[(1-Ethyl-1H-pyrazol-5-yl)methyl]-3,7-dimethyl-6-(naphthalen-2-yl)-4-oxo-4,5-dihydropyrazolo-[1,5-a]pyrazine-2-carboxamide

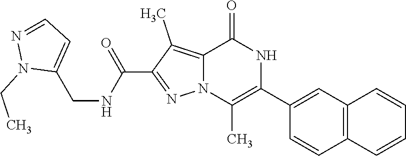 [SMILES: CCn1nccc1CNC(=O)c1nn2c(C)c(-c3ccc4ccccc4c3)[nH]c(=O)c2c1C]

A mixture of 3,7-dimethyl-6-(naphthalen-2-yl)-4-oxo-4,5-dihydropyrazolo[1,5-a]pyrazine-2-carboxylic acid (Intermediate 75A, 100 mg, 300 μmol), HATU (137 mg, 360 μmol) and N,N-diisopropylethylamine (160 μl, 900 μmol) in DMF (2.5 ml) was stirred at RT for 30 min. Then, 1-(1-ethyl-1H-pyrazol-5-yl)methanamine (41.3 mg, 330 μmol) was added, and stirring was continued at RT overnight. The reaction mixture was directly purified by preparative HPLC (Method P11) to afford the title compound. Yield: 87.6 mg (66% of theory).

LC/MS [Method 3]: Rₜ=1.77 min; MS (ESIpos): m/z=441 [M+H]⁺.

¹H-NMR (500 MHz, DMSO-d₆): δ [ppm]=11.44 (s, 1H), 8.78 (t, 1H), 8.09 (s, 1H), 8.06-7.98 (m, 3H), 7.64-7.58 (m, 3H), 7.33 (d, 1H), 6.17 (d, 1H), 4.54 (d, 2H), 4.20 (q, 2H), 2.68 (s, 3H), 2.40 (s, 3H), 1.32 (t, 3H).

### Example 1-69

### N-[(1S)-1-(4-Fluorophenyl)-3-hydroxypropyl]-6-(naphthalen-2-yl)-4-oxo-4,5-dihydropyrazolo-[1,5-a]pyrazine-2-carboxamide

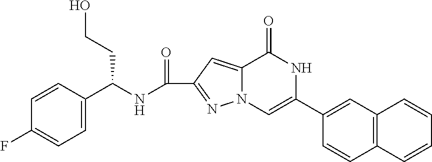 [SMILES: O=C(N[C@@H](CCO)c1ccc(F)cc1)c1cc2c(=O)[nH]c(-c3ccc4ccccc4c3)cn2n1]

The title compound was prepared according to general procedure 1-A starting from 6-(naphthalen-2-yl)-4-oxo-4,5-dihydropyrazolo[1,5-a]pyrazine-2-carboxylic acid (Intermediate 3A, 90.0 mg, 295 μmol), (3S)-3-amino-3-(4-fluorophenyl)propan-1-ol (54.9 mg, 324 μmol), HATU (52.6 mg, 324 μmol) and N,N-diisopropylethylamine (150 μl, 880 μmol) in N-methylpyrrolidone (2.0 ml). After cooling to RT, the mixture was directly purified by preparative HPLC (Method P2) to afford the title compound. Yield: 35.3 mg (25% of theory, 95% purity).

LC/MS [Method 3]: Rₜ=1.67 min; MS (ESIpos): m/z=457 [M+H]⁺.

¹H-NMR (400 MHz, DMSO-d₆): δ [ppm]=11.87 (s, 1H), 8.91 (d, 1H), 8.38 (s, 1H), 8.18 (s, 1H), 8.04 (d, 1H), 8.02-7.96 (m, 2H), 7.88 (dd, 1H), 7.64-7.57 (m, 2H), 7.48-7.41 (m, 2H), 7.38 (s, 1H), 7.19-7.13 (m, 2H), 5.23-5.17 (m, 1H), 4.58 (br. s, 1H), 3.49-3.40 (m, 2H), 2.13-2.03 (m, 1H), 1.98-1.89 (m, 1H).

### Example 1-70

### 6-(3,4-Dimethylphenyl)-N-[(1S)-1-(4-fluorophenyl)-3-hydroxpropyl]-4-oxo-4,5-dihydropyrazolo-[1,5-a]pyrazine-2-carboxamide

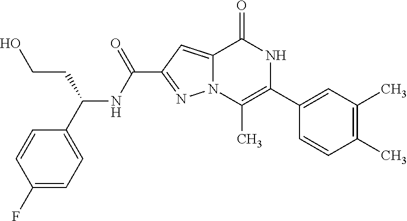 [SMILES: Cc1ccc(-c2[nH]c(=O)c3cc(C(=O)N[C@@H](CCO)c4ccc(F)cc4)nn3c2C)cc1C]

The title compound was prepared according to general procedure 1-B starting from 6-(3,4-di-methylphenyl)-4-oxo-4,5-dihydropyrazolo[1,5-a]pyrazine-2-carboxylic acid (Intermediate 23A, 100 mg, 353 μmol), (3S)-3-amino-3-(4-fluorophenyl)propan-1-ol (65.7 mg, 388 μmol), CDI (63.0 mg, 388 μmol) and DMAP (21.6 mg, 176 μmol) in N-methylpyrrolidone (3.0 ml, 31 mmol). After cooling to RT, the mixture was directly purified by preparative HPLC (Method P2) to afford the title compound. Yield: 55.0 mg (26% of theory).

LC/MS [Method 3]: Rₜ=1.67 min; MS (ESIpos): m/z=435 [M+H]⁺.

¹H-NMR (400 MHz, DMSO-d₆): δ [ppm]=11.65 (s, 1H), 8.87 (d, 1H), 7.95 (s, 1H), 7.56 (s, 1H), 7.49-7.41 (m, 3H), 7.33 (s, 1H), 7.26 (d, 1H), 7.18-7.12 (m, 2H), 5.22-5.15 (m, 1H), 4.62 (t, 1H), 3.46-3.37 (m, 2H), 2.29 (s, 3H), 2.27 (s, 3H), 2.12-2.02 (m, 1H), 1.97-1.88 (m, 1H).

### Example 1-71

### 3-Cyano-N-[(1R)-1-(4-fluorophenyl)-3-hydroxypropyl]-6-(naphthalen-2-yl)-4-oxo-4,5-dihydropyrazolo[1,5-a]pyrazine-2-carboxamide

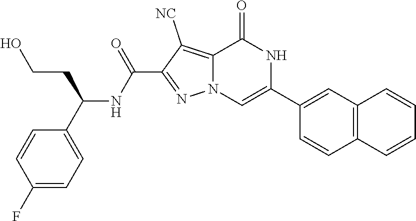 [SMILES: N#Cc1c(C(=O)N[C@H](CCO)c2ccc(F)cc2)nn2cc(-c3ccc4ccccc4c3)[nH]c(=O)c12]

The title compound was prepared according to general procedure 1-B starting from 3-cyano-6-(naphthalen-2-yl)-4-oxo-4,5-dihydropyrazolo[1,5-a]pyrazine-2-carboxylic acid (Intermediate 111A, 100 mg, 303 μmol), (3R)-3-amino-3-(4-fluorophenyl)propan-1-ol (56.3 mg, 333 μmol), CDI (54.0 mg, 333 μmol) and DMAP (18.5 mg, 151 μmol) in N-methylpyrrolidone (2.5 ml). After cooling to RT, the mixture was directly purified by preparative HPLC (Method P5) to afford the title compound. Yield: 44.0 mg (29% of theory, 96% purity).

LC/MS [Method 3]: Rₜ=1.73 min; MS (ESIneg): m/z=480 [M−H]⁻.

¹H-NMR (400 MHz, DMSO-d₆): δ [ppm]=12.45 (br. s, 1H), 9.21 (d, 1H), 8.40 (s, 1H), 8.26 (s, 1H), 8.06 (d, 1H), 8.03-7.97 (m, 2H), 7.89 (dd, 1H), 7.65-7.59 (m, 2H), 7.52-7.44 (m, 2H), 7.20-7.14 (m, 2H), 5.24-5.17 (m, 1H), 4.66 (t, 1H), 3.50-3.37 (m, 2H), 2.15-2.06 (m, 1H), 1.99-1.89 (m, 1H).

### Example 1-72

### 3-Cyano-N-[(1S)-1-(4-fluorophenyl)-3-hydroxpropyl]-6-(naphthalen-2-yl)-4-oxo-4,5-dihydropyrazolo[1,5-a]pyrazine-2-carboxamide

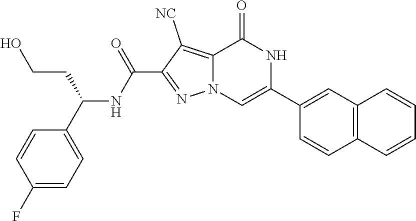 [SMILES: N#Cc1c(C(=O)N[C@@H](CCO)c2ccc(F)cc2)nn2cc(-c3ccc4ccccc4c3)[nH]c(=O)c12]

The title compound was prepared according to general procedure 1-B starting from 3-cyano-6-(naphthalen-2-yl)-4-oxo-4,5-dihydropyrazolo[1,5-a]pyrazine-2-carboxylic acid (Intermediate 111A, 100 mg, 303 μmol), (3S)-3-amino-3-(4-fluorophenyl)propan-1-ol (56.3 mg, 333 μmol), CDI (54.0 mg, 333 μmol) and DMAP (18.5 mg, 151 μmol) in N-methylpyrrolidone (2.5 ml). After cooling to RT, the mixture was directly purified by preparative HPLC (Method P5) to afford the title compound. Yield: 37.0 mg (24% of theory, 95% purity).

LC/MS [Method 3]: Rₜ=1.73 min; MS (ESIpos): m/z=482 [M+H]⁺.

¹H-NMR (400 MHz, DMSO-d₆): δ [ppm]=12.54 (br. s, 1H), 9.18 (d, 1H), 8.42 (s, 1H), 8.25 (s, 1H), 8.04 (d, 1H), 8.02-7.96 (m, 2H), 7.91 (dd, 1H), 7.64-7.58 (m, 2H), 7.50-7.42 (m, 2H), 7.20-7.13 (m, 2H), 5.24-5.16 (m, 1H), 4.66 (br. s, 1H), 3.47-3.39 (m, 2H), 2.15-2.04 (m, 1H), 1.99-1.89 (m, 1H).

### Example 1-73

### 3-Cyano-6-(3,4-dimethylphenyl)-N-[(1R)-1-(4-fluorophenyl)ethyl]-4-oxo-4,5-dihydropyrazolo-[1,5-a]pyrazine-2-carboxamide

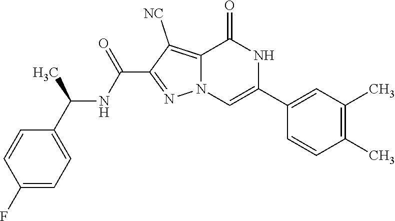 [SMILES: Cc1ccc(-c2cn3nc(C(=O)N[C@H](C)c4ccc(F)cc4)c(C#N)c3c(=O)[nH]2)cc1C]

The title compound was prepared according to general procedure 1-B starting from 3-cyano-6-(3,4-dimethylphenyl)-4-oxo-4,5-dihydropyrazolo[1,5-a]pyrazine-2-carboxylic acid (Intermediate 108A, 100 mg, 324 μmol), (1R)-1-(4-fluorophenyl)ethan-1-amine (49.7 mg, 357 μmol), CDI (57.9 mg, 357 μmol) and DMAP (19.8 mg, 162 μmol) in N-methylpyrrolidone (2.7 ml). After cooling to RT, the mixture was diluted with DMSO and directly purified by preparative HPLC (Method P5) to afford the title compound. Yield: 20.0 mg (13% of theory, 94% purity).

LC/MS [Method 11]: Rₜ=3.34 min; MS (ESIpos): m/z=430 [M+H]⁺.

¹H-NMR (400 MHz, DMSO-d₆): δ [ppm]=12.20 (br. s, 1H), 9.15 (d, 1H), 8.04 (s, 1H), 7.56 (s, 1H), 7.51-7.44 (m, 3H), 7.28 (d, 1H), 7.20-7.13 (m, 2H), 5.22-5.13 (m, 1H), 2.29 (s, 3H), 2.28 (s, 3H), 1.50 (d, 3H).

### Example 1-74

### 3-Cyano-6-(3,4-dimethylphenyl)-N-[(1S)-2-methoxy-1-phenylethyl]-4-oxo-4,5-dihydropyrazolo-[1,5-a]pyrazine-2-carboxamide

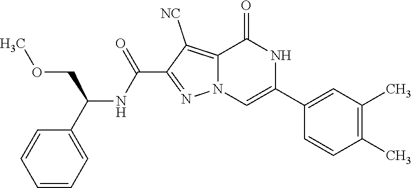 [SMILES: COC[C@@H](NC(=O)c1nn2cc(-c3ccc(C)c(C)c3)[nH]c(=O)c2c1C#N)c1ccccc1]

The title compound was prepared according to general procedure 1-B starting from 3-cyano-6-(3,4-dimethylphenyl)-4-oxo-4,5-dihydropyrazolo[1,5-a]pyrazine-2-carboxylic acid (Intermediate 108A, 100 mg, 324 μmol), (1S)-2-methoxy-1-phenylethan-1-amine (54.0 mg, 357 μmol), CDI (57.9 mg, 357 μmol) and DMAP (19.8 mg, 162 μmol) in N-methylpyrrolidone (2.7 ml). After cooling to RT, the mixture was diluted with DMSO and directly purified by preparative HPLC (Method P5) to afford the title compound. Yield: 34.5 mg (24% of theory).

LC/MS [Method 7]: Rₜ=1.01 min; MS (ESIpos): m/z=442 [M+H]⁺.

¹H-NMR (400 MHz, DMSO-d₆): δ [ppm]=12.19 (br. s, 1H), 9.04 (d, 1H), 8.09 (s, 1H), 7.58 (s, 1H), 7.49 (dd, 1H), 7.47-7.42 (m, 2H), 7.38-7.31 (m, 2H), 7.31-7.25 (m, 2H), 5.29-5.23 (m, 1H), 3.79 (dd, 1H), 3.59 (dd, 1H), 3.30 (s, 3H), 2.29 (s, 3H), 2.28 (s, 3H).

### Example 1-75

### 3-Cyano-6-(3,4-dimethylphenyl)-N-[(1S)-1-(4-fluorophenyl)-3-hydroxpropyl]-4-oxo-4,5-dihydropyrazolo[1,5-a]pyrazine-2-carboxamide

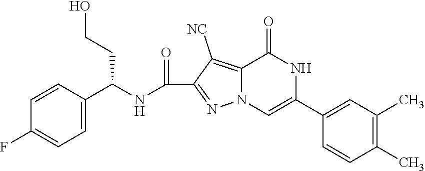 [SMILES: Cc1ccc(-c2cn3nc(C(=O)N[C@@H](CCO)c4ccc(F)cc4)c(C#N)c3c(=O)[nH]2)cc1C]

The title compound was prepared according to general procedure 1-B starting from 3-cyano-6-(3,4-dimethylphenyl)-4-oxo-4,5-dihydropyrazolo[1,5-a]pyrazine-2-carboxylic acid (Intermediate 108A, 100 mg, 324 μmol), (3S)-3-amino-3-(4-fluorophenyl)propan-1-ol (60.4 mg, 357 μmol), CDI (57.9 mg, 357 μmol) and DMAP (19.8 mg, 162 μmol) in N-methylpyrrolidone (2.7 ml). After cooling to RT, the mixture was diluted with DMSO and directly purified by preparative HPLC (Method P5) to afford the title compound. Yield: 25.0 mg (17% of theory).

LC/MS [Method 3]: Rₜ=1.70 min; MS (ESIneg): m/z=458 [M−H]⁻.

¹H-NMR (400 MHz, DMSO-d₆): δ [ppm]=12.19 (br. s, 1H), 9.18 (d, 1H), 8.05 (s, 1H), 7.57 (s, 1H), 7.52-7.42 (m, 3H), 7.28 (d, 1H), 7.20-7.13 (m, 2H), 5.22-5.16 (m, 1H), 4.65 (t, 1H), 3.48-3.34 (m, 2H), 2.29 (s, 3H), 2.28 (s, 3H), 2.14-2.04 (m, 1H), 1.98-1.88 (m, 1H).

### Example 1-76

### N-[(1R)-3-Hydroxy-1-phenylpropyl]-3-(methoxymethyl)-6-(naphthalen-2-yl)-4-oxo-4,5-dihydropyrazolo[1,5-a]pyrazine-2-carboxamide

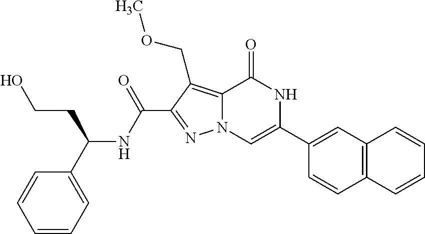 [SMILES: COCc1c(C(=O)N[C@H](CCO)c2ccccc2)nn2cc(-c3ccc4ccccc4c3)[nH]c(=O)c12]

The title compound was prepared according to general procedure 1-B starting from 3-(methoxymethyl)-6-(naphthalen-2-yl)-4-oxo-4,5-dihydropyrazolo[1,5-a]pyrazine-2-carboxylic acid (Intermediate 42A, 100 mg, 286 μmol), (3R)-3-amino-3-phenylpropan-1-ol (47.6 mg, 315 μmol), CDI (51.1 mg, 315 μmol) and DMAP (17.5 mg, 143 μmol) in N-methylpyrrolidone (2.1 ml). After cooling to RT, the mixture was directly purified by preparative HPLC (Method P2) to afford the title compound. Yield: 59.0 mg (43% of theory).

LC/MS [Method 3]: Rₜ=1.74 min; MS (ESIpos): m/z=483 [M+H]⁺.

¹H-NMR (400 MHz, DMSO-d₆): δ [ppm]=11.83 (s, 1H), 8.81 (d, 1H), 8.39 (s, 1H), 8.19 (s, 1H), 8.03 (d, 1H), 8.01-7.95 (m, 2H), 7.89 (dd, 1H), 7.64-7.56 (m, 2H), 7.44-7.38 (m, 2H), 7.38-7.31 (m, 2H), 7.29-7.18 (m, 1H), 5.23-5.15 (m, 1H), 4.93 (s, 2H), 3.59-3.32 (m, 3H), 3.23 (s, 3H), 2.11-1.90 (m, 2H).

### Example 1-77

### N-[(1S)-2-Hydroxy-1-phenylethyl]-6-(naphthalen-2-yl)-4-oxo-3-(trifluoromethyl)-4,5-dihydropyrazolo[1,5-a]pyrazine-2-carboxamide

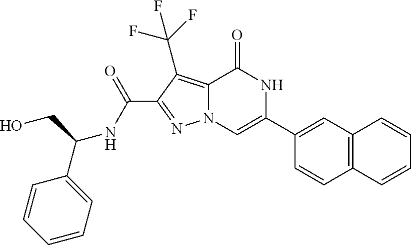 [SMILES: O=C(N[C@H](CO)c1ccccc1)c1nn2cc(-c3ccc4ccccc4c3)[nH]c(=O)c2c1C(F)(F)F]

The title compound was prepared according to general procedure 1-B starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), (2S)-2-amino-2-phenylethan-1-ol (40.4 mg, 295 μmol), CDI (47.8 mg, 295 μmol) and DMAP (16.4 mg, 134 μmol) in N-methylpyrrolidone (2.0 ml). After cooling to RT, the mixture was directly purified by preparative HPLC (Method P2) to afford the title compound. Yield: 79.0 mg (60% of theory).

LC/MS [Method 3]: Rₜ=1.75 min; MS (ESIneg): m/z=491 [M−H]⁻.

¹H-NMR (400 MHz, DMSO-d₆): δ [ppm]=12.23 (s, 1H), 9.10 (d, 1H), 8.41 (d, 2H), 8.05 (d, 1H), 8.02-7.97 (m, 2H), 7.90 (dd, 1H), 7.64-7.59 (m, 2H), 7.42-7.30 (m, 4H), 7.30-7.24 (m, 1H), 5.10-4.98 (m, 1H), 3.67 (d, 2H).

### Example 1-78

### N-[(1S,2S)-2-Hydroxy-2,3-dihydro-1H-inden-1-yl]-6-(naphthalen-2-yl)-4-oxo-3-(trifluoromethyl)-4,5-dihydropyrazolo[1,5-a]pyrazine-2-carboxamide

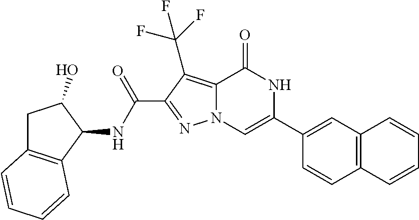 [SMILES: O=C(N[C@H]1c2ccccc2C[C@@H]1O)c1nn2cc(-c3ccc4ccccc4c3)[nH]c(=O)c2c1C(F)(F)F]

The title compound was prepared according to general procedure 1-B starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), (1S,2S)-1-amino-2,3-dihydro-1H-inden-2-ol (44.0 mg, 295 μmol), CDI (47.8 mg, 295 μmol) and DMAP (16.4 mg, 134 μmol) in N-methylpyrrolidone (2.0 ml). After cooling to RT, the mixture was directly purified by preparative HPLC (Method P2) to afford the title compound. Yield: 29.0 mg (21% of theory).

LC/MS [Method 3]: Rₜ=1.82 min; MS (ESIneg): m/z=503 [M−H]⁻.

¹H-NMR (400 MHz, DMSO-d₆): δ [ppm]=12.24 (s, 1H), 9.15 (d, 1H), 8.41 (s, 1H), 8.37 (s, 1H), 8.05 (d, 1H), 8.03-7.97 (m, 2H), 7.89 (dd, 1H), 7.64-7.58 (m, 2H), 7.26-7.18 (m, 4H), 5.38 (d, 1H), 5.29-5.23 (m, 1H), 4.42-4.34 (m, 1H), 3.22-3.15 (m, 1H), 2.76 (dd, 1H).

### Example 1-79

### N-[(1R)-1-(6-Methoxpyridin-3-yl)ethyl]-6-(naphthalen-2-yl)-4-oxo-3-(trifluoromethyl)-4,5-dihydro-pyrazolo[1,5-a]pyrazine-2-carboxamide

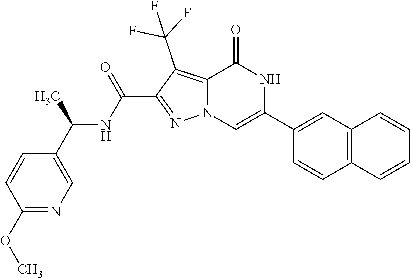 [SMILES: COc1ccc([C@@H](C)NC(=O)c2nn3cc(-c4ccc5ccccc5c4)[nH]c(=O)c3c2C(F)(F)F)cn1]

The title compound was prepared according to general procedure 1-B starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), (1R)-1-(6-methoxypyridin-3-yl)ethan-1-amine (Intermediate 49A, 44.8 mg, 295 μmol), CDI (47.8 mg, 295 μmol) and DMAP (16.4 mg, 134 μmol) in N-methylpyrrolidone (2.2 ml). After cooling to RT, the mixture was directly purified by preparative HPLC (Method P2) to afford the title compound. Yield: 64.7 mg (48% of theory).

LC/MS [Method 7]: Rₜ=0.99 min; MS (ESIpos): m/z=508 [M+H]⁺.

¹H-NMR (400 MHz, DMSO-d₆): δ [ppm]=12.24 (s, 1H), 9.28 (d, 1H), 8.42-8.37 (m, 2H), 8.19 (d, 1H), 8.05 (d, 1H), 8.02-7.97 (m, 2H), 7.89 (dd, 1H), 7.74 (dd, 1H), 7.64-7.59 (m, 2H), 6.83 (d, 1H), 5.18-5.11 (m, 1H), 3.84 (s, 3H), 1.46 (d, 3H).

### Example 1-80

### 6-(3,4-Dimethylphenyl)-N-[(1S,2S)-2-hydroxy-2,3-dihydro-1H-inden-1-yl]-4-oxo-4,5-dihydropyrazolo[1,5-a]pyrazine-2-carboxamide

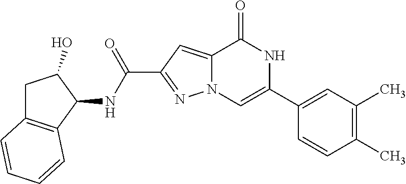 [SMILES: Cc1ccc(-c2cn3nc(C(=O)N[C@H]4c5ccccc5C[C@@H]4O)cc3c(=O)[nH]2)cc1C]

The title compound was prepared according to general procedure 1-B starting from 6-(3,4-dimethylphenyl)-4-oxo-4,5-dihydropyrazolo[1,5-a]pyrazine-2-carboxylic acid (Intermediate 23A, 100 mg, 353 μmol), (1S,2S)-1-amino-2,3-dihydro-1H-inden-2-ol (57.9 mg, 388 μmol), CDI (63.0 mg, 388 μmol) and DMAP (21.6 mg, 176 μmol) in N-methylpyrrolidone (2.6 ml). After cooling to RT, the mixture was directly purified by preparative HPLC (Method P2) to afford the title compound. Yield: 42.0 mg (29% of theory).

LC/MS [Method 3]: Rₜ=1.70 min; MS (ESIneg): m/z=413 [M−H]⁻.

¹H-NMR (400 MHz, DMSO-d₆): δ [ppm]=11.64 (br. s, 1H), 8.68 (d, 1H), 7.96 (s, 1H), 7.56 (s, 1H), 7.47 (dd, 1H), 7.43 (s, 1H), 7.25 (d, 1H), 7.23-7.10 (m, 4H), 5.35 (d, 1H), 5.32-5.26 (m, 1H), 4.53-4.46 (m, 1H), 3.23-3.14 (m, 1H), 2.75 (dd, 1H), 2.29 (s, 3H), 2.27 (s, 3H).

### Example 1-81

### 3-Cyclopropyl-N-[(1R)-1-(4-fluorophenyhethyl]-6-(naphthalen-2-yl)-4-oxo-4,5-dihydropyrazolo-[1,5-a]pyrazine-2-carboxamide

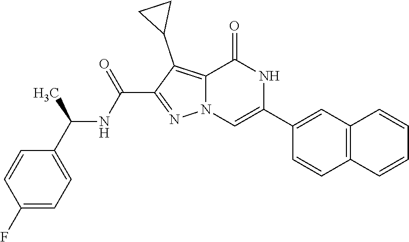 [SMILES: C[C@@H](NC(=O)c1nn2cc(-c3ccc4ccccc4c3)[nH]c(=O)c2c1C1CC1)c1ccc(F)cc1]

The title compound was prepared according to general procedure 1-B starting from 3-cyclopropyl-6-(naphthalen-2-yl)-4-oxo-4,5-dihydropyrazolo[1,5-a]pyrazine-2-carboxylic acid (Intermediate 82A, 100 mg, 290 μmol), CDI (51.6 mg, 319 μmol) and DMAP (17.7 mg, 145 μmol) in N-methyl-pyrrolidone (2.4 ml). After cooling to RT, the mixture was diluted with DMSO and directly purified by preparative HPLC (Method P11) to afford the title compound. Yield: 55.0 mg (41% of theory).

LC/MS [Method 3]: Rₜ=2.25 min; MS (ESIneg): m/z=465 [M−H]⁻.

¹H-NMR (400 MHz, DMSO-d₆): δ [ppm]=11.58 (br. s, 1H), 8.74 (d, 1H), 8.37 (s, 1H), 8.12 (s, 1H), 8.01 (d, 1H), 8.00-7.94 (m, 2H), 7.87 (dd, 1H), 7.62-7.56 (m, 2H), 7.49-7.43 (m, 2H), 7.20-7.14 (m, 2H), 5.19-5.11 (m, 1H), 2.80-2.72 (m, 1H), 1.48 (d, 3H), 1.21-1.07 (m, 2H), 0.88-0.83 (m, 2H).

### Example 1-82

### 3-Cyclopropyl-N-[3-(4-fluorophenyl)-1-methylazetidin-3-yl]-6-(naphthalen-2-yl)-4-oxo-4,5-dihydropyrazolo[1,5-a]pyrazine-2-carboxamide

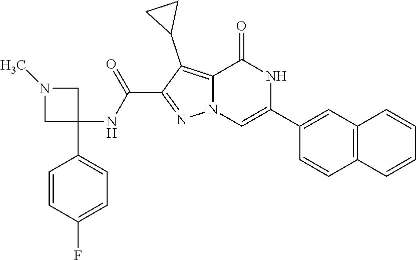 [SMILES: CN1CC(NC(=O)c2nn3cc(-c4ccc5ccccc5c4)[nH]c(=O)c3c2C2CC2)(c2ccc(F)cc2)C1]

The title compound was prepared according to general procedure 1-A starting from 3-cyclopropyl-6-(naphthalen-2-yl)-4-oxo-4,5-dihydropyrazolo[1,5-a]pyrazine-2-carboxylic acid (Intermediate 82A, 75.0 mg, 217 μmol), 3-(4-fluorophenyl)-1-methylazetidin-3-amine (Intermediate 46A, 43.1 mg, 239 μmol), HATU (99.1 mg, 260 μmol) and N,N-diisopropylethylamine (110 μl, 650 μmol) in N-methylpyrrolidone (2.5 ml). After cooling to RT, the mixture was diluted with DMSO and directly purified by preparative HPLC (Method P11). The product-containing fractions were combined and evaporated. The residue was further purified by preparative HPLC (Method P8) to afford the title compound. Yield: 8.9 mg (8% of theory).

LC/MS [Method 3]: Rₜ=1.44 min; MS (ESIneg): m/z=506 [M−H]⁻.

¹H-NMR (400 MHz, DMSO-d₆): δ [ppm]=11.60 (br. s, 1H), 9.34 (s, 1H), 8.37 (s, 1H), 8.10 (s, 1H), 8.02 (d, 1H), 8.00-7.95 (m, 2H), 7.87 (dd, 1H), 7.68-7.62 (m, 2H), 7.62-7.57 (m, 2H), 7.24-7.13 (m, 2H), 3.69 (d, 2H), 3.43 (d, 2H), 2.80-2.72 (m, 1H), 2.33 (s, 3H), 1.18-1.11 (m, 2H), 0.90-0.82 (m, 2H).

### Example 1-83

### 3-Cyclopropyl-6-(3,4-dimethylphenyl)-N-[(1R)-1-(4-fluorophenyl)-3-hydroxpropyl]-4-oxo-4,5-dihydropyrazolo[1,5-a]pyrazine-2-carboxamide

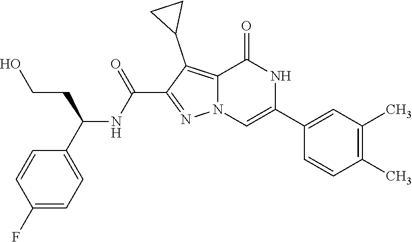 [SMILES: Cc1ccc(-c2cn3nc(C(=O)N[C@H](CCO)c4ccc(F)cc4)c(C4CC4)c3c(=O)[nH]2)cc1C]

The title compound was prepared according to general procedure 1-C starting from 3-cyclopropyl-6-(3,4-dimethylphenyl)-4-oxo-4,5-dihydropyrazolo[1 ,5-a]pyrazine-2-carboxylic acid (Intermediate 92A, 100 mg, 309 μmol), (3R)-3-amino-3-(4-fluorophenyl)propan-1-ol (52.3 mg, 309 μmol), HATU (147 mg, 387 μmol) and N,N-diisopropylethylamine (130 μl, 770 μmol) in DMF (5.0 ml). The reaction mixture was directly purified by preparative HPLC (Method P5) to afford the title compound. Yield: 68.0 mg (46% of theory).

LC/MS [Method 3]: Rₜ=1.91 min; MS (ESIpos): m/z=475 [M+H]⁺.

¹H-NMR (500 MHz, DMSO-d₆): δ [ppm]=11.36 (s, 1H), 8.77 (d, 1H), 7.88 (d, 1H), 7.55 (s, 1H), 7.48-7.40 (m, 3H), 7.23 (d, 1H), 7.19-7.13 (m, 2H), 5.18-5.12 (m, 1H), 4.60 (t, 1H), 3.46-3.34 (m, 2H), 2.78-2.71 (m, 1H), 2.28 (s, 3H), 2.26 (s, 3H), 2.07-1.99 (m, 1H), 1.93-1.86 (m, 1H), 1.17-1.09 (m, 2H), 0.85-0.79 (m, 2H).

### Example 1-84

### 3-Cyclopropyl-6-(3,4-dimethylphenyl)-N-[(1R)-1-(4-fluorophenyl)ethyl]-4-oxo-4,5-dihydropyrazolo[1,5-a]pyrazine-2-carboxamide

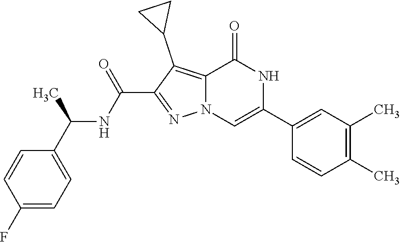 [SMILES: Cc1ccc(-c2cn3nc(C(=O)N[C@H](C)c4ccc(F)cc4)c(C4CC4)c3c(=O)[nH]2)cc1C]

The title compound was prepared according to general procedure 1-C starting from 3-cyclopropyl-6-(3,4-dimethylphenyl)-4-oxo-4,5-dihydropyrazolo[1 ,5-a]pyrazine-2-carboxylic acid (Intermediate 92A, 100 mg, 309 μmol), (1R)-1-(4-fluorophenyl)ethan-1-amine (43.0 mg, 309 μmol), HATU (147 mg, 387 μmol) and N,N-diisopropylethylamine (130 μl, 770 μmol) in DMF (5.0 ml). The reaction mixture was directly purified by preparative HPLC (Method P5) to afford the title compound. Yield: 55.0 mg (40% of theory).

LC/MS [Method 3]: Rₜ=2.25 min; MS (ESIpos): m/z=445 [M+H]⁺.

¹H-NMR (500 MHz, DMSO-d₆): δ [ppm]=11.36 (br. s, 1H), 8.69 (d, 1H), 7.88 (s, 1H), 7.55 (s, 1H), 7.50-7.40 (m, 3H), 7.23 (d, 1H), 7.20-7.12 (m, 2H), 5.17-5.11 (m, 1H), 2.77-2.71 (m, 1H), 2.28 (s, 3H), 2.26 (s, 3H), 1.47 (d, 3H), 1.18-1.09 (m, 2H), 0.87-0.80 (m, 2H).

### Example 1-85

### 6-(3,4-Dimethylphenyl)-N-[2-[ethyl(2-hydroxyethyl)amino]-1-phenylethyl]-4-oxo-5H-pyrazolo-[1,5-a]pyrazine-2-carboxamide

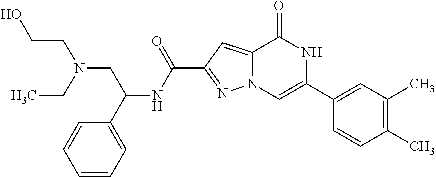 [SMILES: CCN(CCO)CC(NC(=O)c1cc2c(=O)[nH]c(-c3ccc(C)c(C)c3)cn2n1)c1ccccc1]

The title compound was prepared according to general procedure 1-A starting from 6-(3,4-dimethylphenyl)-4-oxo-4,5-dihydropyrazolo[1,5-a]pyrazine-2-carboxylic acid (Intermediate 23A, 80.0 mg, 282 μmol), 2-[(2-amino-2-phenylethyl)ethylamino]ethanol (Intermediate 84A, 64.7 mg, 311 μmol), HATU (129 mg, 340 μmol) and N,N-diisopropylethylamine (150 μl, 850 μmol) in N-methylpyrrolidone (3.2 ml). After cooling to RT, the mixture was diluted with DMSO and directly purified by preparative HPLC (Method P5). The product-containing fractions were combined and evaporated. The residue was further purified by preparative HPLC (Method P7). The product-containing fractions were again combined and evaporated, and the residue was finally purified by column chromatography on basic silica gel (eluent: gradient of methanol in dichloromethane) to afford the title compound. Yield: 10.5 mg (7% of theory).

LC/MS [Method 3]: Rₜ=1.26 min; MS (ESIpos): m/z=474 [M+H]⁺.

¹H-NMR (400 MHz, DMSO-d₆): δ [ppm]=11.65 (br. s, 1H), 8.63 (d, 1H), 7.99 (s, 1H), 7.57 (s, 1H), 7.48 (dd, 1H), 7.43-7.39 (m, 2H), 7.35-7.28 (m, 3H), 7.28-7.21 (m, 2H), 5.13-5.06 (m, 1H), 4.33 (t, 1H), 3.44 (q, 2H), 2.94-2.86 (m, 1H), 2.84-2.75 (m, 1H), 2.65-2.55 (m, 4H), 2.29 (s, 3H), 2.28 (s, 3H), 0.93 (t, 3H).

### Example 1-86

### 6-(3,4-Dimethylphenyl)-N-[3-hydroxy-1-[4-(trifluoromethyl)phenyl]propyl]-4-oxo-5H-pyrazolo-[1,5-a]pyrazine-2-carboxamide

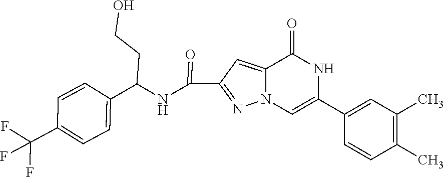 [SMILES: Cc1ccc(-c2cn3nc(C(=O)NC(CCO)c4ccc(C(F)(F)F)cc4)cc3c(=O)[nH]2)cc1C]

The title compound was prepared according to general procedure 1-B starting from 6-(3,4-dimethylphenyl)-4-oxo-4,5-dihydropyrazolo[1,5-a]pyrazine-2-carboxylic acid (Intermediate 23A, 95.0 mg, 335 μmol), 3-amino-3-[4-(trifluoromethyl)phenyl]propan-1-ol hydrochloride (94.3 mg, 369 μmol), CDI (59.8 mg, 369 μmol) and DMAP (20.5 mg, 168 μmol) in N-methylpyrrolidone (2.5 ml). After cooling to RT, the mixture was directly purified by preparative HPLC (Method P2) to afford the title compound. Yield: 77.6 mg (48% of theory).

LC/MS [Method 7]: Rₜ=0.99 min; MS (ESIpos): m/z=485 [M+H]⁺.

¹H-NMR (400 MHz, DMSO-d₆): δ [ppm]=11.66 (s, 1H), 9.01 (d, 1H), 7.96 (s, 1H), 7.73-7.68 (m, 2H), 7.65-7.59 (m, 2H), 7.56 (s, 1H), 7.48 (dd, 1H), 7.34 (s, 1H), 7.26 (d, 1H), 5.29-5.22 (m, 1H), 4.69 (t, 1H), 3.50-3.39 (m, 2H), 2.29 (s, 3H), 2.28 (s, 3H), 2.16-2.06 (m, 1H), 2.00-1.91 (m, 1H).

### Example 1-87

### N-[(1S)-2-Methoxy-1-phenylethyl]-6-(5-methylquinolin-3-yl)-4-oxo-3-(propan-2-yl)-4,5-dihydropyrazolo[1,5-a]pyrazine-2-carboxamide

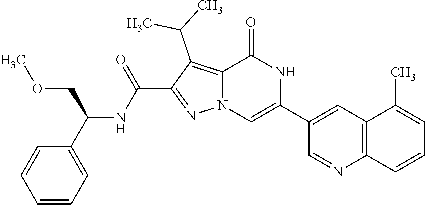 [SMILES: COC[C@@H](NC(=O)c1nn2cc(-c3cnc4cccc(C)c4c3)[nH]c(=O)c2c1C(C)C)c1ccccc1]

The title compound was prepared according to general procedure 1-A starting from 6-(5-methylquinolin-3-yl)-4-oxo-3-(propan-2-yl)-4,5-dihydropyrazolo[1,5-a]pyrazine-2-carboxylic acid (Intermediate 77A, 100 mg, 276 μmol), (1S)-2-methoxy-1-phenylethan-1-amine (45.9 mg, 304 μmol), HATU (126 mg, 331 μmol) and N,N-diisopropylethylamine (140 μl, 830 μmol) in N-methylpyrrolidone (4.5 ml). After cooling to RT, the mixture was filtered, and the filtrate was directly purified by preparative HPLC (Method P8) to afford the title compound. Yield: 32.0 mg (23% of theory, 98% purity).

LC/MS [Method 3]: Rₜ=2.10 min; MS (ESIpos): m/z=496 [M+H]⁺.

¹H-NMR (500 MHz, DMSO-d₆): δ [ppm]=11.88 (br. s, 1H), 9.27 (d, 1H), 8.84 (d, 1H), 8.65 (d, 1H), 8.41 (s, 1H), 7.90 (d, 1H), 7.71 (dd, 1H), 7.52 (d, 1H), 7.47-7.41 (m, 2H), 7.39-7.31 (m, 2H), 7.29-7.25 (m, 1H), 5.28-5.23 (m, 1H), 4.15-4.07 (m, 1H), 3.74 (dd, 1H), 3.61 (dd, 1H), 3.30 (s, 3H), 2.77 (s, 3H), 1.39-1.29 (m, 6H).

### Example 1-88

### N-[(1R)-3-Hydroxy-1-phenylpropyl]-6-(5-methylquinolin-3-yl)-4-oxo-3-(propan-2-yl)-4,5-dihydropyrazolo[1,5-a]pyrazine-2-carboxamide

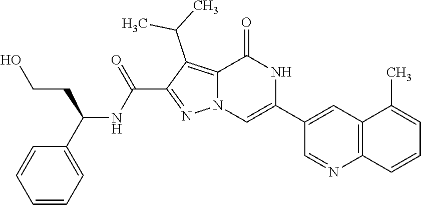 [SMILES: Cc1cccc2ncc(-c3cn4nc(C(=O)N[C@H](CCO)c5ccccc5)c(C(C)C)c4c(=O)[nH]3)cc12]

The title compound was prepared according to general procedure 1-A starting from 6-(5-methylquinolin-3-yl)-4-oxo-3-(propan-2-yl)-4,5-dihydropyrazolo[1,5-a]pyrazine-2-carboxylic acid (Intermediate 77A, 100 mg, 276 μmol), (3R)-3-amino-3-phenylpropan-1-ol (45.9 mg, 304 μmol), HATU (126 mg, 331 μmol) and N,N-diisopropylethylamine (140 μl, 830 μmol) in N-methylpyrrolidone (4.5 ml). After cooling to RT, the mixture was filtered, and the filtrate was directly purified by preparative HPLC (Method P7) to afford the title compound. Yield: 37.0 mg (26% of theory, 97% purity).

LC/MS [Method 3]: Rₜ=1.81 min; MS (ESIpos): m/z=496 [M+H]⁺.

¹H-NMR (400 MHz, DMSO-d₆): δ [ppm]=11.86 (br. s, 1H), 9.26 (d, 1H), 8.83 (d, 1H), 8.79 (d, 1H), 8.39 (s, 1H), 7.90 (d, 1H), 7.71 (dd, 1H), 7.52 (d, 1H), 7.44-7.38 (m, 2H), 7.38-7.31 (m, 2H), 7.28-7.21 (m, 1H), 5.22-5.15 (m, 1H), 4.61 (t, 1H), 4.11-4.02 (m, 1H), 3.49-3.39 (m, 2H), 2.77 (s, 3H), 2.10-2.00 (m, 1H), 1.98-1.88 (m, 1H), 1.39-1.27 (m, 6H).

### Example 1-89

### N-[(1S)-1-(4-Fluorophenyl)-2-hydroxy-2-methylpropyl]-6-(5-methylquinolin-3-yl)-4-oxo-3-(propan-2-yl)-4,5-dihydropyrazolo[1,5-a]pyrazine-2-carboxamide

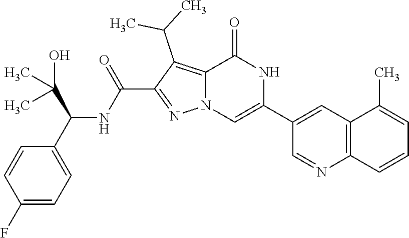 [SMILES: Cc1cccc2ncc(-c3cn4nc(C(=O)N[C@@H](c5ccc(F)cc5)C(C)(C)O)c(C(C)C)c4c(=O)[nH]3)cc12]

The title compound was prepared according to general procedure 1-C starting from 6-(5-methylquinolin-3-yl)-4-oxo-3-(propan-2-yl)-4,5-dihydropyrazolo[1,5-a]pyrazine-2-carboxylic acid (Intermediate 77A, 100 mg, 276 μmol), (1S)-1-amino-1-(4-fluorophenyl)-2-methylpropan-2-ol (Intermediate 36A, 60.7 mg, 331 μmol), HATU (126 mg, 331 μmol) and N,N-diisopropylethylamine (140 μl, 830 μmol) in DMF (2.4 ml). The reaction mixture was directly purified by preparative HPLC (Method P11) to afford the title compound. Yield: 68.2 mg (45% of theory, 95% purity).

LC/MS [Method 3]: Rₜ=2.01 min; MS (ESIpos): m/z=528 [M+H]⁺.

¹H-NMR (500 MHz, DMSO-d₆): δ [ppm]=11.90 (s, 1H), 9.32 (d, 1H), 8.89 (d, 1H), 8.53 (d, 1H), 8.37 (d, 1H), 7.92 (d, 1H), 7.73 (dd, 1H), 7.54 (d, 1H), 7.47-7.42 (m, 2H), 7.17-7.12 (m, 2H), 4.87 (d, 1H), 4.20-4.12 (m, 1H), 2.78 (s, 3H), 1.34 (d, 3H), 1.32 (d, 3H), 1.27 (s, 3H), 0.99 (s, 3H).

### Example 1-90

### 6-(3,4-Dimethylphenyl)-N-[(1R)-1-(4-fluorophenyl)ethyl]-4-oxo-3-(propan-2-yl)-4,5-dihydropyrazolo[1,5-a]pyrazine-2-carboxamide

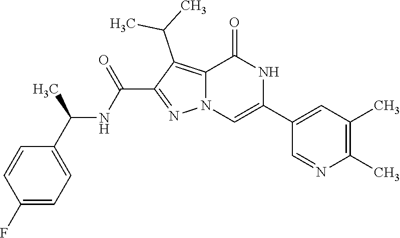 [SMILES: Cc1cc(-c2cn3nc(C(=O)N[C@H](C)c4ccc(F)cc4)c(C(C)C)c3c(=O)[nH]2)cnc1C]

The title compound was prepared according to general procedure 1-C starting from 6-(3,4-dimethylphenyl)-4-oxo-3-(propan-2-yl)-4,5-dihydropyrazolo[1,5-a]pyrazine-2-carboxylic acid (Intermediate 128A, 100 mg, 307 μmol), (1R)-1-(4-fluorophenyl)ethan-1-amine (42.8 mg, 307 μmol), HATU (146 mg, 384 μmol) and N,N-diisopropylethylamine (130 μl, 770 μmol) in DMF (3.3 ml). The reaction mixture was directly purified by preparative HPLC (Method P5) to afford the title compound. Yield: 28.0 mg (20% of theory, 96% purity).

LC/MS [Method 3]: Rₜ=2.33 min; MS (ESIpos): m/z=447 [M+H]⁺.

¹H-NMR (500 MHz, DMSO-d₆): δ [ppm]=11.42 (br. s, 1H), 8.68 (d, 1H), 7.89 (s, 1H), 7.55 (s, 1H), 7.49-7.42 (m, 3H), 7.24 (d, 1H), 7.19-7.13 (m, 2H), 5.19-5.12 (m, 1H), 4.08-4.00 (m, 1H), 2.28 (s, 3H), 2.27 (s, 3H), 1.47 (d, 3H), 1.31 (d, 3H), 1.30 (d, 3H).

### Example 1-91

### 6-(3,4-Dimethylphenyl)-N-[3-(4-fluorophenyl)-1-methylazetidin-3-yl]-4-oxo-3-(propan-2-yl)-4,5-dihydropyrazolo[1,5-a]pyrazine-2-carboxamide

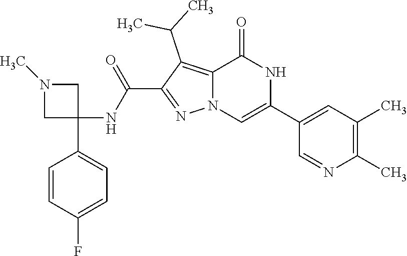

The title compound was prepared according to general procedure 1-C starting from 6-(3,4-di-methylphenyl)-4-oxo-3-(propan-2-yl)-4,5-dihydropyrazolo[1,5-a]pyrazine-2-carboxylic acid (Intermediate 128A, 100 mg, 307 μmol), 3-(4-fluorophenyl)-1-methylazetidin-3-amine (Intermediate 46A, 55.4 mg, 307 μmol), HATU (146 mg, 384 μmol) and N,N-diisopropylethylamine (130 μl, 770 μmol) in DMF (3.3 ml). The reaction mixture was directly purified by preparative HPLC (Method P5) to afford the title compound. Yield: 70.0 mg (47% of theory).

LC/MS [Method 3]: Rₜ=1.47 min; MS (ESIpos): m/z=488 [M+H]⁺.

¹H-NMR (500 MHz, DMSO-d₆): δ [ppm]=11.47 (br. s, 1H), 9.46 (s, 1H), 7.83 (s, 1H), 7.66-7.60 (m, 2H), 7.54 (s, 1H), 7.46 (dd, 1H), 7.28-7.18 (m, 3H), 4.09-3.98 (m, 3H), 3.92 (br. s, 2H), 2.61-2.52 (m, 3H), 2.29 (s, 3H), 2.27 (s, 3H), 1.29 (d, 6H).

### Example 1-92

### 6-(3,4-Dimethylphenyl)-N-[(1R)-1-(4-fluorophenyl)-3-hydroxpropyl]-4-oxo-3-(propan-2-yl)-4,5-dihydropyrazolo[1,5-a]pyrazine-2-carboxamide

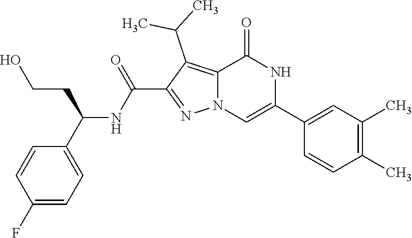 [SMILES: Cc1ccc(-c2cn3nc(C(=O)N[C@H](CCO)c4ccc(F)cc4)c(C(C)C)c3c(=O)[nH]2)cc1C]

The title compound was prepared according to general procedure 1-C starting from 6-(3,4-di-methylphenyl)-4-oxo-3-(propan-2-yl)-4,5-dihydropyrazolo[1,5-a]pyrazine-2-carboxylic acid (Intermediate 128A, 100 mg, 307 μmol), (3R)-3-amino-3-(4-fluorophenyl)propan-1-ol (52.0 mg, 307 μmol), HATU (146 mg, 384 μmol) and N,N-diisopropylethylamine (130 μl, 770 μmol) in DMF (3.3 ml). The reaction mixture was directly purified by preparative HPLC (Method P5) to afford the title compound. Yield: 77.0 mg (53% of theory).

LC/MS [Method 3]: Rₜ=2.01 min; MS (ESIpos): m/z=477 [M+H]⁺.

¹H-NMR (500 MHz, DMSO-d₆): δ [ppm]=11.41 (br. s, 1H), 8.77 (d, 1H), 7.89 (s, 1H), 7.56 (s, 1H), 7.49-7.41 (m, 3H), 7.24 (d, 1H), 7.19-7.13 (m, 2H), 5.20-5.14 (m, 1H), 4.64-4.58 (m, 1H), 4.08-4.00 (m, 1H), 3.47-3.38 (m, 2H), 2.28 (s, 3H), 2.27 (s, 3H), 2.08-1.99 (m, 1H), 1.94-1.86 (m, 1H), 1.31 (d, 3H), 1.29 (d, 3H).

### Example 1-93

### 6-(3,4-Dimethylphenyl)-N-[3-(4-fluorophenyl)-1-methylazetidin-3-yl]-4-oxo-3-(trifluoromethyl)-4,5-dihydropyrazolo[1,5-a]pyrazine-2-carboxamide

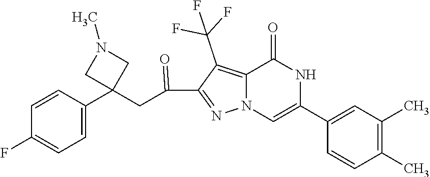 [SMILES: Cc1ccc(-c2cn3nc(C(=O)CC4(c5ccc(F)cc5)CN(C)C4)c(C(F)(F)F)c3c(=O)[nH]2)cc1C]

The title compound was prepared according to general procedure 1-C starting from 6-(3,4-dimethylphenyl)-4-oxo-3-(trifluoromethyl)-4,5-dihydropyrazolo[1,5-a]pyrazine-2-carboxylic acid (Intermediate 125A, 120 mg, 342 μmol), 3-(4-fluorophenyl)-1-methylazetidin-3-amine (Intermediate 46A, 67.7 mg, 376 μmol), HATU (156 mg, 410 μmol) and N,N-diisopropylethylamine (180 μl, 1.03 mmol) in DMF (3.0 ml). The reaction mixture was diluted with DMSO and directly purified by preparative HPLC (Method P11) to afford the title compound. Yield: 71.2 mg (41% of theory).

LC/MS [Method 3]: Rₜ=1.32 min; MS (ESIpos): m/z=514 [M+H]⁺.

¹H-NMR (500 MHz, DMSO-d₆): δ [ppm]=12.03 (br. s, 1H), 9.74 (s, 1H), 8.19 (s, 1H), 7.66-7.61 (m, 2H), 7.59 (s, 1H), 7.50 (dd, 1H), 7.27 (d, 1H), 7.23-7.17 (m, 2H), 3.69 (d, 2H), 3.43 (d, 2H), 2.34 (s, 3H), 2.29 (s, 3H), 2.28 (s, 3H).

### Example 1-94

### 6-(3,4-Dimethylphenyl)-N-[(1R)-1-(4-fluorophenyl)-3-hydroxpropyl]-4-oxo-3-(trifluoromethyl)-4,5-dihydropyrazolo[1,5-a]pyrazine-2-carboxamide

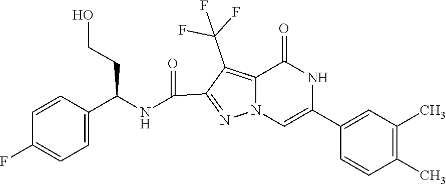 [SMILES: Cc1ccc(-c2cn3nc(C(=O)N[C@H](CCO)c4ccc(F)cc4)c(C(F)(F)F)c3c(=O)[nH]2)cc1C]

The title compound was prepared according to general procedure 1-C starting from 6-(3,4-dimethylphenyl)-4-oxo-3-(trifluoromethyl)-4,5-dihydropyrazolo[1,5-a]pyrazine-2-carboxylic acid (Intermediate 125A, 100 mg, 285 μmol), (3R)-3-amino-3-(4-fluorophenyl)propan-1-ol (57.8 mg, 342 μmol), HATU (130 mg, 342 μmol) and N,N-diisopropylethylamine (150 μl, 850 μmol) in DMF (2.5 ml). The reaction mixture was diluted with DMSO and directly purified by preparative HPLC (Method P11) to afford the title compound. Yield: 100 mg (70% of theory).

LC/MS [Method 3]: Rₜ=1.79 min; MS (ESIneg): m/z=501 [M−H]⁻.

¹H-NMR (500 MHz, DMSO-d₆): δ [ppm]=12.02 (br. s, 1H), 9.20 (d, 1H), 8.16 (s, 1H), 7.59 (s, 1H), 7.50 (dd, 1H), 7.44-7.36 (m, 2H), 7.26 (d, 1H), 7.21-7.15 (m, 2H), 5.17-5.11 (m, 1H), 4.57 (t, 1H), 3.48-3.35 (m, 2H), 2.29 (s, 3H), 2.28 (s, 3H), 2.01-1.93 (m, 1H), 1.89-1.81 (m, 1H).

### Example 1-95

### 6-(3,4-Dimethylphenyl)-N-[(1R)-1-(4-fluorophenyl)ethyl]-4-oxo-3-(trifluoromethyl)-4,5-dihydropyrazolo[1,5-a]pyrazine-2-carboxamide

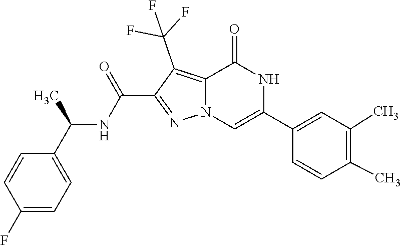 [SMILES: Cc1ccc(-c2cn3nc(C(=O)N[C@H](C)c4ccc(F)cc4)c(C(F)(F)F)c3c(=O)[nH]2)cc1C]

The title compound was prepared according to general procedure 1-C starting from 6-(3,4-dimethylphenyl)-4-oxo-3-(trifluoromethyl)-4,5-dihydropyrazolo[1,5-a]pyrazine-2-carboxylic acid (Intermediate 125A, 100 mg, 285 μmol), (1R)-1-(4-fluorophenyl)ethan-1-amine (46 μl, 340 μmol), HATU (130 mg, 342 μmol) and N,N-diisopropylethylamine (150 μl, 850 μmol) in DMF (2.5 ml). The reaction mixture was diluted with DMSO and directly purified by preparative HPLC (Method P11) to afford the title compound. Yield: 82.5 mg (61% of theory).

LC/MS [Method 3]: Rₜ=2.06 min; MS (ESIpos): m/z=473 [M+H]⁺. ¹H-NMR (500 MHz, DMSO-d₆): δ [ppm]=12.03 (br. s, 1H), 9.23 (d, 1H), 8.16 (s, 1H), 7.59 (s, 1H), 7.50 (dd, 1H), 7.46-7.39 (m, 2H), 7.26 (d, 1H), 7.21-7.15 (m, 2H), 5.18-5.12 (m, 1H), 2.29 (s, 3H), 2.28 (s, 3H), 1.44 (d, 3H).

### Example 1-96

### 6-(2,3-Dihydro-1H-inden-5-yl)-N-[3-(4-fluorophenyl)-1-methylazetidin-3-yl]-4-oxo-4,5-dihydropyrazolo[1,5-a]pyrazine-2-carboxamide

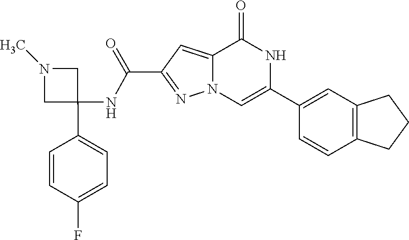 [SMILES: CN1CC(NC(=O)c2cc3c(=O)[nH]c(-c4ccc5c(c4)CCC5)cn3n2)(c2ccc(F)cc2)C1]

The title compound was prepared according to general procedure 1-C starting from 6-(2,3-dihydro-1H-inden-5-yl)-4-oxo-4,5-dihydropyrazolo[1,5-a]pyrazine-2-carboxylic acid (Intermediate 132A, 90.0 mg, 305 μmol), 3-(4-fluorophenyl)-1-methylazetidin-3-amine (Intermediate 46A, 54.9 mg, 305 μmol), HATU (145 mg, 381 μmol) and N,N-diisopropylethylamine (130 μl, 760 μmol) in DMF (3.2 ml). The reaction mixture was directly purified by preparative HPLC (Method P9). The product-containing fractions were combined and evaporated. The residue was further purified by preparative HPLC (Method P5) to afford the title compound. Yield: 7.9 mg (5% of theory).

LC/MS [Method 7]: Rₜ=0.73 min; MS (ESIpos): m/z=458 [M+H]⁺.

¹H-NMR (500 MHz, DMSO-d₆): δ [ppm]=11.50 (br. s, 1H), 9.41 (s, 1H), 7.89 (s, 1H), 7.67-7.56 (m, 3H), 7.49 (dd, 1H), 7.37-7.31 (m, 2H), 7.18-7.13 (m, 2H), 3.69 (d, 2H), 3.45 (d, 2H), 2.94-2.89 (m, 4H), 2.32 (s, 3H), 2.10-2.03 (m, 2H).

### Example 1-97

### 6-(2,3-Dihydro-1H-inden-5-yl)-N-[(1R)-1-(4-fluorophenyl)-3-hydroxpropyl]-4-oxo-4,5-dihydropyrazolo[1,5-a]pyrazine-2-carboxamide

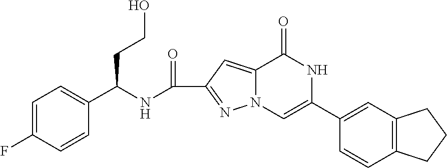 [SMILES: O=C(N[C@H](CCO)c1ccc(F)cc1)c1cc2c(=O)[nH]c(-c3ccc4c(c3)CCC4)cn2n1]

The title compound was prepared according to general procedure 1-C starting from 6-(2,3-dihydro-1H-inden-5-yl)-4-oxo-4,5-dihydropyrazolo[1,5-a]pyrazine-2-carboxylic acid (Intermediate 132A, 90.0 mg, 305 μmol), (3R)-3-amino-3-(4-fluorophenyl)propan-1-ol (51.6 mg, 305 μmol), HATU (145 mg, 381 μmol) and N,N-diisopropylethylamine (130 μl, 760 μmol) in DMF (3.2 ml). The reaction mixture was directly purified by preparative HPLC (Method P9) to afford the title compound. Yield: 19.0 mg (14% of theory).

LC/MS [Method 3]: Rₜ=1.73 min; MS (ESIpos): m/z=447 [M+H]⁺.

¹H-NMR (500 MHz, DMSO-d₆): δ [ppm]=11.67 (s, 1H), 8.88 (d, 1H), 7.92 (s, 1H), 7.60 (s, 1H), 7.49 (dd, 1H), 7.47-7.42 (m, 2H), 7.36-7.33 (m, 2H), 7.18-7.12 (m, 2H), 5.21-5.16 (m, 1H), 4.62 (t, 1H), 3.46-3.37 (m, 2H), 2.94-2.88 (m, 4H), 2.10-2.03 (m, 3H), 1.96-1.88 (m, 1H).

### Example 1-98

### 6-(2,3-Dihydro-1H-inden-5-yl)-N-[(1R)-1-(4-fluorophenyl)ethyl]-4-oxo-4,5-dihydropyrazolo[1,5-a]-pyrazine-2-carboxamide

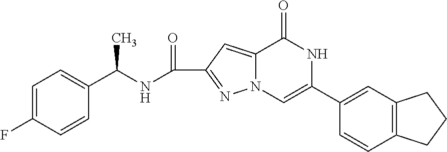 [SMILES: C[C@@H](NC(=O)c1cc2c(=O)[nH]c(-c3ccc4c(c3)CCC4)cn2n1)c1ccc(F)cc1]

The title compound was prepared according to general procedure 1-C starting from 6-(2,3-dihydro-1H-inden-5-yl)-4-oxo-4,5-dihydropyrazolo[1,5-a]pyrazine-2-carboxylic acid (Intermediate 132A, 90.0 mg, 305 μmol), (1R)-1-(4-fluorophenyl)ethan-1-amine (42.4 mg, 305 μmol), HATU (145 mg, 381 μmol) and N,N-diisopropylethylamine (130 μl, 760 μmol) in DMF (3.2 ml). The reaction mixture was directly purified by preparative HPLC (Method P9) to afford the title compound. Yield: 17.0 mg (12% of theory, 91% purity).

LC/MS [Method 3]: Rₜ=2.00 min; MS (ESIpos): m/z=417 [M+H]⁺.

¹H-NMR (500 MHz, DMSO-d₆): δ [ppm]=11.67 (br. s, 1H), 8.82 (d, 1H), 7.91 (s, 1H), 7.59 (s, 1H), 7.50-7.42 (m, 3H), 7.38-7.32 (m, 2H), 7.18-7.12 (m, 2H), 5.21-5.14 (m, 1H), 2.94-2.88 (m, 4H), 2.09-2.02 (m, 2H), 1.49 (d, 3H).

### Example 1-99

### N-[3-(4-Fluorophenyl)-1-methylazetidin-3-yl]-4-oxo-6-(5,6,7,8-tetrahydronaphthalen-2-yl)-4,5-dihydropyrazolo[1,5-a]pyrazine-2-carboxamide

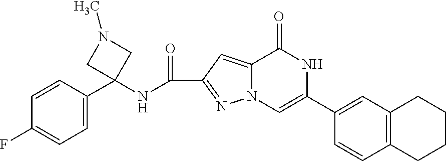 [SMILES: CN1CC(NC(=O)c2cc3c(=O)[nH]c(-c4ccc5c(c4)CCCC5)cn3n2)(c2ccc(F)cc2)C1]

The title compound was prepared according to general procedure 1-C starting from 4-oxo-6-(5,6,7,8-tetrahydronaphthalen-2-yl)-4,5-dihydropyrazolo[1,5-a]pyrazine-2-carboxylic acid (Intermediate 136A, 90.0 mg, 291 μmol), 3-(4-fluorophenyl)-1-methylazetidin-3-amine (Intermediate 46A, 52.4 mg, 291 μmol), HATU (138 mg, 364 μmol) and N,N-diisopropylethylamine (130 μl, 730 μmol) in DMF (3.1 ml). The reaction mixture was directly purified by preparative HPLC (Method P7) to afford the title compound. Yield: 26.0 mg (17% of theory, 90% purity).

LC/MS [Method 3]: Rₜ=1.37 min; MS (ESIpos): m/z=472 [M+H]⁺.

¹H-NMR (500 MHz, DMSO-d₆): δ [ppm]=11.67 (br. s, 1H), 9.41 (s, 1H), 7.91 (s, 1H), 7.66-7.58 (m, 2H), 7.49-7.42 (m, 2H), 7.33-7.31 (m, 1H), 7.20-7.12 (m, 3H), 3.69 (d, 2H), 3.45 (d, 2H), 2.77 (br. dd, 4H), 2.32 (s, 3H), 1.79-1.73 (m, 4H).

### Example 1-100

### N-[(1R)-1-(4-Fluorophenyl)-3-hydroxypropyl]-4-oxo-6-(5,6,7,8-tetrahydronaphthalen-2-yl)-4,5-dihydropyrazolo[1,5-a]pyrazine-2-carboxamide

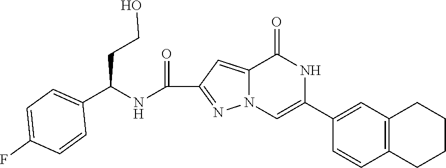 [SMILES: O=C(N[C@H](CCO)c1ccc(F)cc1)c1cc2c(=O)[nH]c(-c3ccc4c(c3)CCCC4)cn2n1]

The title compound was prepared according to general procedure 1-C starting from 4-oxo-6-(5,6,7,8-tetrahydronaphthalen-2-yl)-4,5-dihydropyrazolo[1,5-a]pyrazine-2-carboxylic acid (Intermediate 136A, 90.0 mg, 291 μmol), (3R)-3-amino-3-(4-fluorophenyl)propan-1-ol (49.2 mg, 291 μmol), HATU (138 mg, 364 μmol) and N,N-diisopropylethylamine (130 μl, 730 μmol) in DMF (3.1 ml). The reaction mixture was directly purified by preparative HPLC (Method P9) to afford the title compound. Yield: 32.5 mg (23% of theory, 95% purity).

LC/MS [Method 3]: Rₜ=1.83 min; MS (ESIpos): m/z=461 [M+H]⁺.

¹H-NMR (500 MHz, DMSO-d₆): δ [ppm]=11.66 (br. s, 1H), 8.87 (d, 1H), 7.93 (s, 1H), 7.49-7.41 (m, 4H), 7.33 (d, 1H), 7.20-7.11 (m, 3H), 5.21-5.15 (m, 1H), 4.62 (t, 1H), 3.48-3.37 (m, 2H), 2.81-2.73 (m, 4H), 2.11-2.01 (m, 1H), 1.96-1.87 (m, 1H), 1.80-1.72 (m, 4H).

### Example 1-101

### N-[(1R)-1-(4-Fluorophenyl)ethyl]-4-oxo-6-(5,6,7,8-tetrahydronaphthalen-2-yl)-4,5-dihydropyrazolo[1,5-a]pyrazine-2-carboxamide

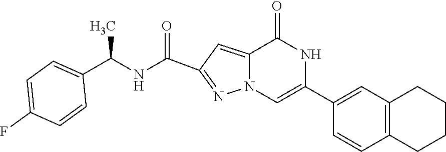 [SMILES: C[C@@H](NC(=O)c1cc2c(=O)[nH]c(-c3ccc4c(c3)CCCC4)cn2n1)c1ccc(F)cc1]

The title compound was prepared according to general procedure 1-C starting from 4-oxo-6-(5,6,7,8-tetrahydronaphthalen-2-yl)-4,5-dihydropyrazolo[1,5-a]pyrazine-2-carboxylic acid (Intermediate 136A, 90.0 mg, 291 μmol), (1R)-1-(4-fluorophenyl)ethan-1-amine (40.5 mg, 291 μmol), HATU (138 mg, 364 μmol) and N,N-diisopropylethylamine (130 μl, 730 μmol) in DMF (3.1 ml). The reaction mixture was directly purified by preparative HPLC (Method P7) to afford the title compound. Yield: 18.0 mg (14% of theory).

LC/MS [Method 7]: Rₜ=1.10 min; MS (ESIpos): m/z=431 [M+H]⁺.

¹H-NMR (500 MHz, DMSO-d₆): δ [ppm]=11.65 (s, 1H), 8.80 (d, 1H), 7.93 (s, 1H), 7.48-7.42 (m, 4H), 7.36 (d, 1H), 7.18-7.12 (m, 3H), 5.21-5.14 (m, 1H), 2.81-2.73 (m, 4H), 1.79-1.73 (m, 4H), 1.49 (d, 3H).

### Example 1-102

### N-[(1R)-1-(4-Fluorophenyl)ethyl]-6-(4-methyl-3,4-dihydro-2H-1,4-benzoxazin-6-yl)-4-oxo-4,5-dihydropyrazolo[1,5-a]pyrazin-2-carboxamide

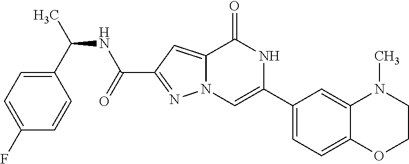 [SMILES: C[C@@H](NC(=O)c1cc2c(=O)[nH]c(-c3ccc4c(c3)N(C)CCO4)cn2n1)c1ccc(F)cc1]

6-(4-Methyl-3,4-dihydro-2H-1,4-benzoxazin-6-yl)-4-oxo-4,5-dihydropyrazolo[1,5-a]pyrazine-2-carboxylic acid (Intermediate 31A, 200 mg, 55% purity, 337 μmol) was dissolved in DMF (8.0 ml). N,N-Diisopropylethylamine (180 μl, 1.0 mmol), HATU (141 mg, 371 μmol) and (1R)-1-(4-fluorophenyl)-ethanamine (46.9 mg, 337 μmol) were added in this order, and the mixture was stirred overnight at ambient temperature. LC/MS indicated insufficient product formation, so additional (1R)-1-(4-fluorophenyl)ethanamine (9.4 mg, 67.4 μmol) was added, and the reaction mixture was further stirred at RT over the weekend. Product formation was still insufficient, so additional HATU (70 mg, 186 μmol) and (1R)-1-(4-fluorophenyl)ethanamine (23.5 mg, 168 μmol) were added, and the mixture was stirred for further 4 h. Then, the reaction mixture was partitioned between water and dichloromethane. The organic layer was recovered, and the aqueous phase was extracted with dichloromethane. The combined organic phases were washed with brine and dried over sodium sulfate. Removal of solvent under reduced pressure gave 592.3 mg of crude product which was dissolved in DMSO and purified by preparative HPLC (Method P13; gradient: A 30%/B 70% to A 0%/B 100% in 7 min, 150 ml/min, two runs). Combination and lyophilisation of the appropriate fractions gave the title compound. Yield: 146.5 mg (97% of theory).

LC/MS [Method 34]: Rₜ=1.15 min; MS (ESIpos): m/z=448 [M+H]⁺.

¹H-NMR (500 MHz, DMSO-d₆): δ [ppm]=11.63 (s, 1H), 8.81 (d, 1H), 7.93 (s, 1H), 7.42-7.49 (m, 2H), 7.34 (s, 1H), 7.10-7.19 (m, 2H), 7.03 (d, 1H), 6.96 (dd, 1H), 6.75 (d, 1H), 5.17 (dq, 1H), 4.22-4.30 (m, 2H), 3.24-3.33 (m, 2H), 2.92 (s, 3H), 1.49 (d, 3H).

### Example 1-103

### N-(3,4-Dimethoxybenzyl)-6-(2-naphthyl)-4-oxo-4,5-dihydropyrazolo[1,5-a]pyrazin-2-carboxamide

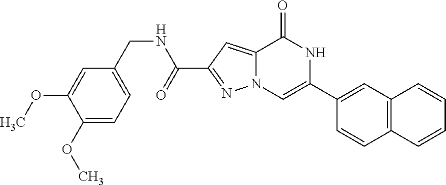 [SMILES: COc1ccc(CNC(=O)c2cc3c(=O)[nH]c(-c4ccc5ccccc5c4)cn3n2)cc1OC]

6-(2-Naphthyl)-4-oxo-4,5-dihydropyrazolo[1,5-a]pyrazine-2-carboxylic acid (Intermediate 3A, 200 mg, 655 μmol) was dissolved in DMF (10 ml). HATU (274 mg, 721 μmol) and N,N-diisopropylethylamine (340 μl, 2.0 mmol) were added, and the mixture was stirred at RT for 1 h. Then, 1-(3,4-dimethoxphenyl)methanamine (110 mg, 655 μmol) was added, and the mixture was further stirred at RT overnight. After this, the reaction mixture was partitioned between water and dichloromethane. The organic layer was recovered, and the aqueous phase was extracted with dichloromethane. The combined organic phases were washed with brine and dried over sodium sulfate. Removal of solvent under reduced pressure gave 353 mg of crude product which was dissolved in DMSO and purified by preparative HPLC (Method P12; gradient: A 85%/B 15% to A 0%/B 100% in 7 min, 150 ml/min, two runs). Combination and lyophilisation of the appropriate fractions gave the title compound. Yield: 47 mg (15% of theory).

LC/MS [Method 34]: Rₜ=1.11 min; MS (ESIpos): m/z=455 [M+H]⁺.

¹H-NMR (500 MHz, DMSO-d₆): δ [ppm]=11.89 (br. s, 1H), 8.94 (t, 1H), 8.37 (d, 1H), 8.17 (s, 1H), 8.03 (d, 1H), 7.95-8.00 (m, 2H), 7.87 (dd, 1H), 7.56-7.63 (m, 2H), 7.40 (d, 1H), 6.98 (d, 1H), 6.90 (d, 1H), 6.86 (dd, 1H), 4.42 (d, 2H), 3.74 (s, 3H), 3.72 (s, 3H).

### Example 1-104

### N-[1-(6-Methompyridin-3-yl)ethyl]-6-(2-naphthyl)-4-oxo-5H-pyrazolo[1,5-a]pyrazine-2-carboxamide

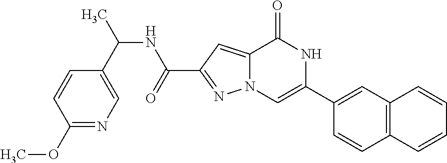 [SMILES: COc1ccc(C(C)NC(=O)c2cc3c(=O)[nH]c(-c4ccc5ccccc5c4)cn3n2)cn1]

6-(2-Naphthyl)-4-oxo-4,5-dihydropyrazolo[1 ,5-a]pyrazine-2-carboxylic acid (Intermediate 3A, 200 mg, 655 μmol) was dissolved in DMF (10.0 ml). N,N-Diisopropylethylamine (340 μl, 2.0 mmol), HATU (274 mg, 721 μmol) and 1-(6-methoxypyridin-3-yl)ethanamine (99.7 mg, 655 μmol) were added, and the mixture was stirred at RT overnight. Then, the reaction mixture was partitioned between water and dichloromethane. The organic layer was recovered, and the aqueous phase was extracted with dichloromethane. The combined organic phases were washed with brine and dried over sodium sulfate. Removal of solvent under reduced pressure gave 537 mg of crude product which was dissolved in DMSO and purified by preparative HPLC (Method P13; gradient: A 70%/B 30% to A 30%/B 70% in 6 min, 150 ml/min, three runs). Combination and lyophilisation of the appropriate fractions afforded the title compound. Yield: 122.7 mg (41% of theory).

LC/MS [Method 34]: Rₜ=1.13 min; MS (ESIpos): m/z=440 [M+H]⁺.

¹H-NMR (400 MHz, DMSO-d₆): δ [ppm]=11.89 (br. s, 1H), 8.89 (d, 1H), 8.37 (d, 1H), 8.20 (d, 1H), 8.17 (s, 1H), 8.03 (d, 1H), 7.95-8.01 (m, 2H), 7.86 (dd, 1H), 7.80 (dd, 1H), 7.56-7.63 (m, 2H), 7.40 (d, 1H), 6.80 (d, 1H), 5.17 (dq, 1H), 3.82 (s, 3H), 1.51 (d, 3H).

### Example 1-105

### N-[(1R)-1-(4-Fluorophenyl)ethyl]-4-oxo-6-(quinolin-2-yl)-4,5-dihydropyrazolo[1,5-a]pyrazine-2-carboxamide

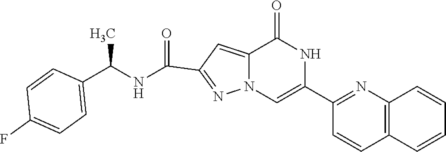 [SMILES: C[C@@H](NC(=O)c1cc2c(=O)[nH]c(-c3ccc4ccccc4n3)cn2n1)c1ccc(F)cc1]

A solution of 4-oxo-6-(quinolin-2-yl)-4,5-dihydropyrazolo[1,5-a]pyrazine-2-carboxylic acid (Intermediate 167A, 50.0 mg, 88% purity, 144 μmol) and (1R)-1-(4-fluorophenyl)ethan-1-amine (29 μl, 220 μmol) in pyridine (500 pl) was treated with T3P (50% in ethyl acetate, 260 μl, 430 μmol) at RT and then heated to 50° C. for 45 min. After this, the reaction mixture was diluted with 2 ml dichloromethane and filtered. The solid was washed with dichloromethane, water and acetone and dried under high vacuum to provide the title product. Yield: 36.1 mg (59% of theroy).

LC/MS [Method 3]: Rₜ=1.89 min; MS (ESIpos): m/z=428 [M+H]⁺.

¹H-NMR (400 MHz, DMSO-d₆): δ [ppm]=11.10 (br. s, 1H), 8.91-8.85 (m, 2H), 8.54 (d, 1H), 8.30 (d, 1H), 8.18 (d, 1H), 8.05 (d, 1H), 7.86 (t, 1H), 7.68 (t, 1H), 7.51-7.45 (m, 3H), 7.16 (t, 2H), 5.20 (quin, 1H), 1.51 (d, 3H).

### Example 1-106

### N-[1-(6-Methoxypyridin-3-yl)ethyl]-4-oxo-6-(quinolin-2-yl)-4,5-dihydropyrazolo[1,5-a]pyrazine-2-carboxamide

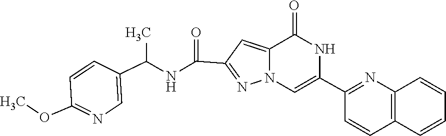 [SMILES: COc1ccc(C(C)NC(=O)c2cc3c(=O)[nH]c(-c4ccc5ccccc5n4)cn3n2)cn1]

A solution of 4-oxo-6-(quinolin-2-yl)-4,5-dihydropyrazolo[1,5-a]pyrazine-2-carboxylic acid (Intermediate 167A, 50.0 mg, 88% purity, 144 μmol) and 1-(6-methoxypyridin-3-yl)ethan-1-amine (32.8 mg, 215 μmol) in pyridine (500 μl) was treated with T3P (50% in ethyl acetate, 260 μl, 430 μmol) at RT and then heated to 50° C. for 30 min. After this, the reaction mixture was diluted with 2 ml dichloromethane and filtered. The solid was washed with dichloromethane, water and acetone and dried under high vacuum to provide the title product. Yield: 38.1 mg (60% of theory).

LC/MS [Method 7]: Rₜ=0.90 min; MS (ESIpos): m/z=441 [M+H]⁺.

¹H-NMR (400 MHz, DMSO-d₆): δ [ppm]=11.10 (s, 1H), 8.91-8.85 (m, 2H), 8.54 (d, 1H), 8.30 (d, 1H), 8.22-8.16 (m, 2H), 8.05 (d, 1H), 7.85 (t, 1H), 7.80 (dd, 1H), 7.68 (t, 1H), 7.48 (s, 1H), 6.81 (d, 1H), 5.18 (quin, 1H), 3.83 (s, 3H), 1.53 (d, 3H).

### Example 1-107

### 6-(4-Chloro-3-methylphenyl)-N-[1-(6-methoxypyridin-3-yl)ethyl]-4-oxo-4,5-dihydropyrazolo[1,5-a]-pyrazine-2-carboxamide

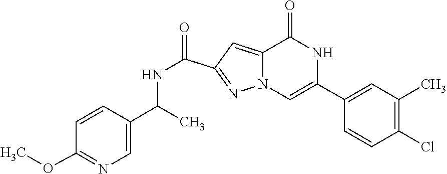 [SMILES: COc1ccc(C(C)NC(=O)c2cc3c(=O)[nH]c(-c4ccc(Cl)c(C)c4)cn3n2)cn1]

A solution of 6-(4-chloro-3-methylphenyl)-4-oxo-4,5-dihydropyrazolo[1,5-a]pyrazine-2-carboxylic acid (Intermediate 170A, 50.0 mg, 165 μmol) and 1-(6-methoxypyridin-3-yl)ethan-1-amine (37.6 mg, 247 μmol) was treated with T3P (50% in ethyl acetate, 290 μl, 490 μmol) at RT and then heated to 50° C. for 30 min. After this, the reaction mixture was directly purified by chromatography over silica gel (eluent: 0-10% methanol in dichloromethane) to afford the title compound. Yield: 47.1 mg (65% of theory).

LC/MS [Method 3]: Rₜ=1.75 min; MS (ESIpos): m/z=438 [M+H]⁺.

¹H-NMR (400 MHz, DMSO-d₆): δ [ppm]=11.71 (br. s, 1H), 8.83 (d, 1H), 8.19 (d, 1H), 8.04 (s, 1H), 7.81-7.76 (m, 2H), 7.62-7.56 (m, 1H), 7.56-7.52 (m, 1H), 7.37 (s, 1H), 6.80 (d, 1H), 5.16 (quin, 1H), 3.82 (s, 3H), 2.40 (s, 3H), 1.51 (d, 3H).

### Example 1-108

### 6-(5-Fluoro-2,3-dihydro-1,4-benzodioxin-6-yl)-N-[(1R)-1-(6-methoxpyridin-3-yl)ethyl]-4-oxo-4,5-dihydropyrazolo[1,5-a]pyrazine-2-carboxamide

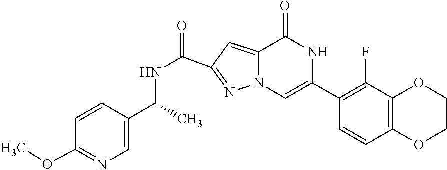 [SMILES: COc1ccc([C@@H](C)NC(=O)c2cc3c(=O)[nH]c(-c4ccc5c(c4F)OCCO5)cn3n2)cn1]

A solution of 6-(5-fluoro-2,3-dihydro-1,4-benzodioxin-6-yl)-4-oxo-4,5-dihydropyrazolo[1,5-a]-pyrazine-2-carboxylic acid (Intermediate 177A, 45.0 mg, 97% purity, 132 μmol) and (1R)-1-(6-methoxypyridin-3-yl)ethan-1-amine hydrochloride (Intermediate 178A, 44.5 mg, 198 μmol) in pyridine (1.0 ml) was treated with T3P (50% in ethyl acetate, 230 μl, 400 μmol) at RT and then heated to 50° C. for 30 min. Three more equivalents of T3P were added, and the mixture was further stirred for 30 min at 90° C. After cooling, the reaction mixture was directly purified by chromatography over silica gel eluting with dichloromethane/0-10% methanol to give the title compound. Yield: 26.3 mg (43% of theory).

LC/MS [Method 7]: Rₜ=0.80 min; MS (ESIpos): m/z=466 [M+H]⁺.

¹H-NMR (400 MHz, DMSO-d₆): δ [ppm]=11.60 (br. s, 1H), 8.84 (br. d, 1H), 8.18 (d, 1H), 7.78 (d, 1H), 7.76 (s, 1H), 7.31-7.24 (m, 1H), 7.16-7.08 (m, 1H), 6.83 (dd, 1H), 6.79 (d, 1H), 5.15 (quin, 1H), 4.35 (s, 4H), 3.82 (s, 3H), 1.50 (d, 3H).

### Example 1-109

### N-[(1S)-2-(Dimethylamino)-1-phenylethyl]-6-(5-fluoro-2,3-dihydro-1,4-benzodioxin-6-yl)-4-oxo-4,5-dihydropyrazolo[1,5-a]pyrazine-2-carboxamide

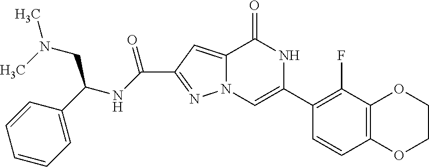 [SMILES: CN(C)C[C@@H](NC(=O)c1cc2c(=O)[nH]c(-c3ccc4c(c3F)OCCO4)cn2n1)c1ccccc1]

A solution of 6-(5-fluoro-2,3-dihydro-1,4-benzodioxin-6-yl)-4-oxo-4,5-dihydropyrazolo[1,5-a]pyrazine-2-carboxylic acid (Intermediate 177A, 45.0 mg, 97% purity, 132 μmol) and (1S)-N²,N²-dimethyl-1-phenylethane-1,2-diamine (32.5 mg, 198 μmol) in pyridine (1 ml) was treated with T3P (50% in ethyl acetate, 230 μl, 400 μmol) at RT and then heated to 90° C. for 30 min. After cooling, the reaction mixture was directly purified by chromatography over silica gel eluting with dichloromethane/0-10% methanol to give the title compound. Yield: 35.0 mg (56% of theory).

LC/MS [Method 7]: Rₜ=0.62 min; MS (ESIpos): m/z=478 [M+H]⁺.

¹H-NMR (400 MHz, DMSO-d₆): δ [ppm]=11.69 (br. s, 1H), 8.67 (br. d, 1H), 7.80 (s, 1H), 7.42 (d, 2H), 7.35-7.29 (m, 3H), 7.26-7.21 (m, 1H), 7.07 (t, 1H), 6.85 (dd, 1H), 5.17-5.10 (m, 1H), 4.36 (s, 4H), 2.85 (br. t, 1H), 2.41 (br. dd, 1H), 2.21 (s, 6H).

### Example 1-110

### N-(2,2-Difluoro-1-phenylethyl)-6-(5-fluoro-2,3-dihydro-1,4-benzodioxin-6-yl)-4-oxo-4,5-dihydro-pyrazolo[1,5-a]pyrazine-2-carboxamide

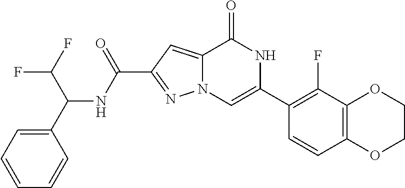 [SMILES: O=C(NC(c1ccccc1)C(F)F)c1cc2c(=O)[nH]c(-c3ccc4c(c3F)OCCO4)cn2n1]

A solution of 6-(5-fluoro-2,3-dihydro-1,4-benzodioxin-6-yl)-4-oxo-4,5-dihydropyrazolo[1,5-a]-pyrazine-2-carboxylic acid (Intermediate 177A, 43.0 mg, 97% purity, 126 μmol), 2,2-difluoro-1-phenylethan-1-amine (29.7 mg, 189 μmol) and N,N-diisopropylethylamine (59 μl, 340 μmol) in DMF (1.1 ml) was treated with HATU (95.8 mg, 252 μmol) and stirred at RT for 18 h. The reaction mixture was then treated with water and extracted with ethyl acetate three times. The organic phase was evaporated, and the residue was purified by preparative HPLC [Chromatorex C-18, 125×30 mm; eluent: gradient of acetonitrile/water (+0.2% ammonia)]. The material thus obtained was further purified by chromatography over silica gel eluting with dichloromethane/0-10% methanol to provide the title compound. Yield: 26 mg (43% of theory).

LC/MS [Method 7]: Rₜ=0.92 min; MS (ESIpos): m/z=471 [M+H]⁺.

¹H-NMR (400 MHz, DMSO-d₆): δ [ppm]=11.73 (br. s, 1H), 9.31 (d, 1H), 7.80 (s, 1H), 7.56 (d, 2H), 7.43-7.33 (m, 4H), 7.07 (t, 1H), 6.85 (dd, 1H), 6.57-6.22 (m, 1H), 5.51-5.41 (m, 1H), 4.36 (s, 4H).

### Example 1-111

### 7-Fluoro-6-(5-fluoro-2,3-dihydro-1,4-benzodioxin-6-yl)-N-[(1R)-1-(6-methoxypyridin-3-yl)ethyl]-4-oxo-4,5-dihydropyrazolo[1,5-a]pyrazine-2-carboxamide

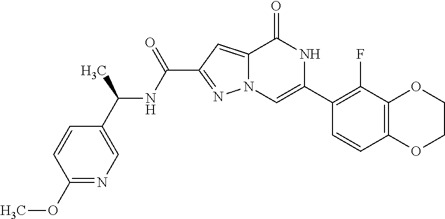 [SMILES: COc1ccc([C@@H](C)NC(=O)c2cc3c(=O)[nH]c(-c4ccc5c(c4F)OCCO5)cn3n2)cn1]

A solution of 7-fluoro-6-(5-fluoro-2,3-dihydro-1,4-benzodioxin-6-yl)-4-oxo-4,5-dihydropyrazolo-[1,5-a]pyrazine-2-carboxylic acid (Intermediate 181A, 11.0 mg, 94% purity, 29.6 μmol), (1R)-1-(6-methoxypyridin-3-yl)ethan-1-amine hydrochloride (Intermediate 178A, 6.14 mg, 32.6 μmol) and N,N-diisopropylethylamine (19 μl, 110 μmol) in DMF (1.0 ml) was treated with HATU (22.5 mg, 59.2 μmol) and stirred at RT for 18 h. The reaction mixture was then treated with water and directly purified by preparative HPLC [Chromatorex C-18, 125×30 mm; eluent: gradient of acetonitrile/water (+0.2% ammonia)]. The material thus obtained was further purified by chromatography over silica gel eluting with dichloromethane/0-10% methanol to provide the title compound. Yield: 5.80 mg (41% of theory.).

LC/MS [Method 3]: Rₜ=1.54 min; MS (ESIpos): m/z=484 [M+H]⁺.

¹H-NMR (400 MHz, DMSO-d₆): δ [ppm]=11.68 (br. s, 1H), 9.01 (d, 1H), 8.18 (d, 1H), 7.79 (dd, 1H), 7.44 (d, 1H), 7.05 (t, 1H), 6.90 (dd, 1H), 6.79 (d, 1H), 5.17 (quin, 1H), 4.37 (s, 4H), 3.82 (s, 3H), 1.52 (d, 3H).

### Example 1-112

### 6-(3,4-Dihydro-1H-2-benzopyran-7-yl)-N-[(1R)-1-(4-fluorophenyl)ethyl]-4-oxo-4,5-dihydropyrazolo[1,5-a]pyrazine-2-carboxamide

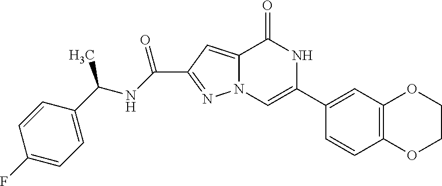 [SMILES: C[C@@H](NC(=O)c1cc2c(=O)[nH]c(-c3ccc4c(c3)OCCO4)cn2n1)c1ccc(F)cc1]

The title compound was prepared according to general procedure 1-B starting from 6-(3,4-dihydro-1H-2-benzopyran-7-yl)-4-oxo-4,5-dihydropyrazolo[1,5-a]pyrazine-2-carboxylic acid (Intermediate 66A, 100 mg, 321 μmol), (1R)-1-(4-fluorophenyl)ethan-1-amine (43 μl, 320 μmol), CDI (104 mg, 642 μmol) and DMAP (19.6 mg, 161 μmol) in N-methylpyrrolidone (4.6 ml). After cooling to RT, the mixture was directly purified by preparative HPLC (Method P2) to afford the title compound. Yield: 103 mg (74% of theory).

LC/MS [Method 3]: Rₜ=1.71 min; MS (ESIpos): m/z=433 [M+H]⁺.

¹H-NMR (500 MHz, DMSO-d₆): δ [ppm]=11.69 (s, 1H), 8.81 (d, 1H), 7.96 (s, 1H), 7.54 (dd, 1H), 7.48-7.42 (m, 3H), 7.37 (d, 1H), 7.26 (d, 1H), 7.18-7.12 (m, 2H), 5.21-5.14 (m, 1H), 4.73 (s, 2H), 3.91 (t, 2H), 2.83 (t, 2H), 1.49 (d, 3H).

### Example 1-113

### N-[(1R)-1-(4-Fluorophenyl)-3-hydroxypropyl]-6-(5-methylquinolin-3-yl)-4-oxo-4,5-dihydropyrazolo[1,5-a]pyrazine-2-carboxamide

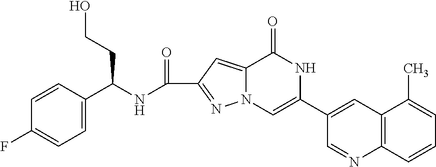 [SMILES: Cc1cccc2ncc(-c3cn4nc(C(=O)N[C@H](CCO)c5ccc(F)cc5)cc4c(=O)[nH]3)cc12]

to The title compound was prepared according to general procedure 1-B starting from 6-(5-methylquinolin-3-yl)-4-oxo-4,5-dihydropyrazolo[1,5-a]pyrazine-2-carboxylic acid (Intermediate 224A, 100 mg, 312 μmol), (3R)-3-amino-3-(4-fluorophenyl)propan-1-ol (58.1 mg, 343 μmol), CDI (55.7 mg, 343 μmol) and DMAP (19.1 mg, 156 μmol) in N-methylpyrrolidone (2.6 ml). After cooling to RT, the mixture was directly purified by preparative HPLC (Method P2) to afford the title compound. Yield: 32.9 mg (22% of theory).

LC/MS [Method 3]: Rₜ=1.49 min; MS (ESIpos): m/z=472 [M+H]⁺.

¹H-NMR (400 MHz, DMSO-d₆): δ [ppm]=12.09 (s, 1H), 9.28 (d, 1H), 8.93 (d, 1H), 8.86 (d, 1H), 8.44 (s, 1H), 7.91 (d, 1H), 7.72 (dd, 1H), 7.53 (d, 1H), 7.48-7.42 (m, 2H), 7.40 (s, 1H), 7.20-7.13 (m, 2H), 5.24-5.17 (m, 1H), 3.47-3.40 (m, 2H), 2.77 (s, 3H), 2.13-2.04 (m, 1H), 1.98-1.89 (m, 1H).

### Example 1-114

### N-[(1R)-3-Hydroxy-1-phenylpropyl]-6-(5-methylquinolin-3-yl)-4-oxo-4,5-dihydropyrazolo[1,5-a]-pyrazine-2-carboxamide

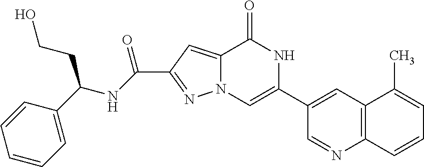 [SMILES: Cc1cccc2ncc(-c3cn4nc(C(=O)N[C@H](CCO)c5ccccc5)cc4c(=O)[nH]3)cc12]

The title compound was prepared according to general procedure 1-B starting from 6-(5-methylquinolin-3-yl)-4-oxo-4,5-dihydropyrazolo[1,5-a]pyrazine-2-carboxylic acid (Intermediate 224A, 100 mg, 312 μmol), (3R)-3-amino-3-phenylpropan-1-ol (51.9 mg, 343 μmol), CDI (55.7 mg, 343 μmol) and DMAP (19.1 mg, 156 μmol) in N-methylpyrrolidone (2.6 ml). After cooling to RT, the mixture was directly purified by preparative HPLC (Method P2) to afford the title compound. Yield: 18.9 mg (13% of theory).

LC/MS [Method 3]: Rₜ=1.46 min; MS (ESIpos): m/z=454 [M+H]⁺.

¹H-NMR (400 MHz, DMSO-d₆): δ [ppm]=12.09 (s, 1H), 9.29 (d, 1H), 8.91-8.84 (m, 2H), 8.45 (s, 1H), 7.92 (d, 1H), 7.73 (dd, 1H), 7.54 (d, 1H), 7.47-7.38 (m, 3H), 7.37-7.31 (m, 2H), 7.28-7.21 (m, 1H), 5.24-5.14 (m, 1H), 3.51-3.34 (m, 2H), 2.77 (s, 3H), 2.15-2.02 (m, 1H), 2.01-1.90 (m, 1H).

### Example 1-115

### N-[2-[Ethyl(2-hydroxyethyl)amino]-1-phenylethyl]-6-(5-methyl-3-quinolyl)-4-oxo-5H-pyrazolo-[1,5-a]pyrazine-2-carboxamide

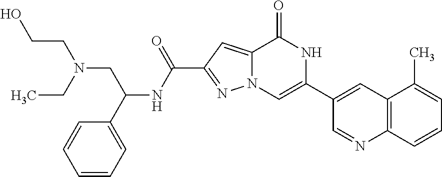 [SMILES: CCN(CCO)CC(NC(=O)c1cc2c(=O)[nH]c(-c3cnc4cccc(C)c4c3)cn2n1)c1ccccc1]

The title compound was prepared according to general procedure 1-A starting from 6-(5-methyl-quinolin-3-yl)-4-oxo-4,5-dihydropyrazolo[1,5-a]pyrazine-2-carboxylic acid (Intermediate 224A, 80.0 mg, 250 μmol), (rac)-2-[(2-Amino-2-phenylethyl)ethylamino]ethanol (Intermediate 85A, 57.2 mg, 275 μmol), HATU (114 mg, 300 μmol) and N,N-diisopropylethylamine (130 μl, 750 μmol) in N-methylpyrrolidone (2.9 ml). After cooling to RT, the reaction mixture was diluted with DMSO and purified by twofold preparative HPLC (first Method P5, then Method P7) to afford the title compound. Yield: 38.5 mg (31% of theory).

LC/MS [Method 3]: Rₜ=1.13 min; MS (ESIpos): m/z=511 [M+H]⁺.

¹H-NMR (400 MHz, DMSO-d₆): δ [ppm]=12.08 (br. s, 1H), 9.28 (d, 1H), 8.85 (d, 1H), 8.67 (d, 1H), 8.47 (s, 1H), 7.91 (d, 1H), 7.72 (dd, 1H), 7.53 (d, 1H), 7.45-7.39 (m, 3H), 7.37-7.30 (m, 2H), 7.28-7.21 (m, 1H), 5.15-5.08 (m, 1H), 4.33 (t, 1H), 3.51-3.39 (m, 2H), 2.98-2.86 (m, 1H), 2.83-2.74 (m, 4H), 2.64-2.56 (m, 4H), 0.94 (t, 3H).

### Example 1-116

### N-[4-(4-Fluorophenyl)-1-methylpiperidin-4-yl]-6-(naphthalen-2-yl)-4-oxo-3-(trifluoromethyl)-4,5-dihydropyrazolo[1,5-a]pyrazine-2-carboxamide

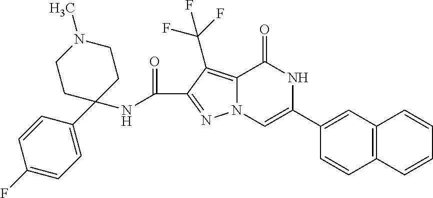 [SMILES: CN1CCC(NC(=O)c2nn3cc(-c4ccc5ccccc5c4)[nH]c(=O)c3c2C(F)(F)F)(c2ccc(F)cc2)CC1]

The title compound was prepared according to general procedure 1-A starting from 6-(naphthalen-2-yl)-4-oxo-3-(trifluoromethyl)-4,5-dihydropyrazolo[1,5-a]pyrazine-2-carboxylic acid (Intermediate 35A, 70.0 mg, 188 μmol), 4-(4-fluorophenyl)-1-methylpiperidin-4-amine (Intermediate 184A, 78.1 mg, 375 μmol), HATU (85.6 mg, 230 μmol) and N,N-diisopropylethylamine (98 μl, 560 μmol) in N-methylpyrrolidone (3.0 ml). After cooling to RT, the mixture was directly purified by preparative HPLC (Method P2). The product-containing fractions were combined and lyophilized. The material thus obtained was eluted over StratoSpheres™ PL-HCO₃ MP resin to afford the title compound. Yield: 57.0 mg (54% of theory).

LC/MS [Method 3]: Rₜ=1.34 min; MS (ESIpos): m/z=564 [M+H]⁺.

¹H-NMR (400 MHz, DMSO-d₆): δ [ppm]=12.21 (br. s, 1H), 8.78 (s, 1H), 8.44 (s, 2H), 8.06-7.92 (m, 4H), 7.64-7.58 (m, 2H), 7.54-7.47 (m, 2H), 7.20-7.13 (m, 2H), 2.71-2.60 (m, 2H), 2.46-2.42 (m, 2H), 2.37-2.24 (m, 2H), 2.21 (s, 3H), 2.01-1.88 (m, 2H).

### Example 1-117

### 6-(2-Naphthyl)-4-oxo-N-[1-[5-(2,2,2-trifluoroethoxy)pyrazin-2-yl]ethyl]-3-(trifluoromethyl)-5H-pyrazolo[1,5-a]pyrazine-2-carboxamide

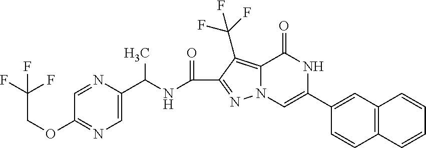 [SMILES: CC(NC(=O)c1nn2cc(-c3ccc4ccccc4c3)[nH]c(=O)c2c1C(F)(F)F)c1cnc(OCC(F)(F)F)cn1]

The title compound was prepared according to general procedure 1-A starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), 1-[5-(2,2,2-trifluoroethoxy)pyrazin-2-yl]ethan-1-amine (71.1 mg, 321 μmol), HATU (122 mg, 321 μmol) and N,N-diisopropylethylamine (140 μl, 800 μmol) in N-methylpyrrolidone (3.1 ml). After cooling to RT, the mixture was directly purified by preparative HPLC (Method P2) to afford the title compound. Yield: 80.2 mg (52% of theory).

LC/MS [Method 3]: Rₜ=2.12 min; MS (ESIpos): m/z=577 [M+H]⁺.

¹H-NMR (400 MHz, DMSO-d₆): δ [ppm]=12.24 (s, 1H), 9.32 (d, 1H), 8.49 (d, 1H), 8.42-8.39 (m, 2H), 8.30 (d, 1H), 8.05 (d, 1H), 8.02-7.97 (m, 2H), 7.89 (dd, 1H), 7.64-7.59 (m, 2H), 5.29-5.21 (m, 1H), 5.06 (q, 2H), 1.52 (d, 3H).

### Example 1-118

### N-[2-(3,5-Dichlorophenoxy)-1-methylethyl]-6-(2-naphthyl)-4-oxo-3-(trifluoromethyl)-5H-pyrazolo-[1,5-a]pyrazine-2-carboxamide

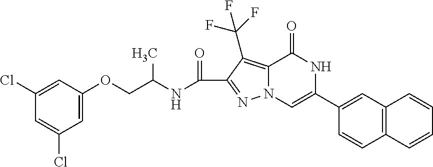 [SMILES: CC(COc1cc(Cl)cc(Cl)c1)NC(=O)c1nn2cc(-c3ccc4ccccc4c3)[nH]c(=O)c2c1C(F)(F)F]

The title compound was prepared according to general procedure 1-A starting from 6-(naphthalen-2-yl)-4-oxo-3-(trifluoromethyl)-4,5-dihydropyrazolo[1,5-a]pyrazine-2-carboxylic acid (Intermediate 35A, 100 mg, 268 μmol), 1-(3,5-dichlorophenoxy)propan-2-amine (70.8 mg, 321 μmol), HATU (122 mg, 321 μmol) and N,N-diisopropylethylamine (140 μl, 800 μmol) in N-methyl-pyrrolidone (3.1 ml). After cooling to RT, the mixture was directly purified by preparative HPLC (Method P2) to afford the title compound. Yield: 67.4 mg (44% of theory).

LC/MS [Method 7]: Rₜ=1.26 min; MS (ESIpos): m/z=575 [M+H]⁺.

¹H-NMR (400 MHz, DMSO-d₆): δ [ppm]=12.23 (s, 1H), 8.86 (d, 1H), 8.40 (s, 1H), 8.35 (s, 1H), 8.05 (d, 1H), 8.02-7.97 (m, 2H), 7.88 (dd, 1H), 7.65-7.58 (m, 2H), 7.19-7.16 (m, 1H), 7.09-7.05 (m, 2H), 4.41-4.31 (m, 1H), 4.07 (d, 2H), 1.25 (d, 3H).

### Example 1-119

### 3-Chloro-N-[3-(4-fluorophenyl)-1-methylazetidin-3-yl]-6-(naphthalen-2-yl)-4-oxo-4,5-dihydropyrazolo[1,5-a]pyrazine-2-carboxamide

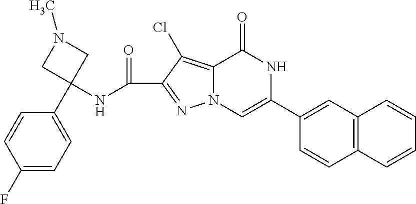 [SMILES: CN1CC(NC(=O)c2nn3cc(-c4ccc5ccccc5c4)[nH]c(=O)c3c2Cl)(c2ccc(F)cc2)C1]

The title compound was prepared according to general procedure 1-A starting from 3-chloro-6-(naphthalen-2-yl)-4-oxo-4,5-dihydropyrazolo[1,5-a]pyrazine-2-carboxylic acid (Intermediate 142A, 100 mg, 294 μmol), 3-(4-fluorophenyl)-1-methylazetidin-3-amine (Intermediate 46A, 58.4 mg, 324 μmol), HATU (134 mg, 353 μmol) and N,N-diisopropylethylamine (150 μl, 880 μmol) in DMF (2.1 ml). After cooling to RT, the mixture was poured into water (100 ml). The precipitate was collected by filtration, washed with water and dried. The solid material was dissolved in dichloromethane and methanol and purified by column chromatography on basic silica gel (eluent: gradient of methanol in dichloromethane) to afford the title compound. Yield: 62.2 mg (41% of theory).

LC/MS [Method 3]: Rₜ=1.31 min; MS (ESIpos): m/z=502 [M+H]⁺.

¹H-NMR (600 MHz, DMSO-d₆): δ [ppm]=11.88 (br. s, 1H), 9.45 (s, 1H), 8.38 (s, 1H), 8.12 (s, 1H), 8.04 (d, 1H), 8.02-7.97 (m, 2H), 7.87 (dd, 1H), 7.66-7.59 (m, 4H), 7.21-7.14 (m, 2H), 3.70 (d, 2H), 3.50-3.41 (m, 2H), 2.33 (s, 3H).

### Example 1-120

### 3-Chloro-N-[(1R)-1-(4-fluorophenyl)-3-hydroxypropyl]-6-(naphthalen-2-yl)-4-oxo-4,5-dihydropyrazolo[1,5-a]pyrazine-2-carboxamide

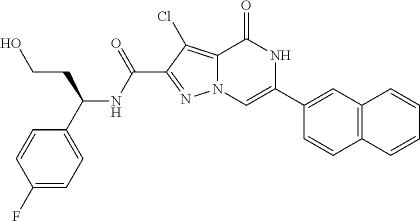 [SMILES: O=C(N[C@H](CCO)c1ccc(F)cc1)c1nn2cc(-c3ccc4ccccc4c3)[nH]c(=O)c2c1Cl]

The title compound was prepared according to general procedure 1-A starting from 3-chloro-6-(naphthalen-2-yl)-4-oxo-4,5-dihydropyrazolo[1,5-a]pyrazine-2-carboxylic acid (Intermediate 142A, 100 mg, 294 μmol), (3R)-3-amino-3-(4-fluorophenyl)propan-1-ol (59.8 mg, 353 μmol), HATU (134 mg, 353 μmol) and N,N-diisopropylethylamine (150 μl, 880 μmol) in DMF (2.1 ml). After cooling to RT, the mixture was poured into water (100 ml). The precipitate was collected by filtration, washed with water/acetonitrile (1:1) and dried. The solid material was dissolved in dichloromethane and methanol and purified by column chromatography on basic silica gel (eluent: gradient of methanol in dichloromethane) to afford the title compound. Yield: 23.8 mg (16% of theory).

LC/MS [Method 3]: Rₜ=1.76 min; MS (ESIneg): m/z=489 [M−H]⁻.

¹H-NMR (500 MHz, DMSO-d₆): δ [ppm]=11.89 (br. s, 1H), 8.95 (d, 1H), 8.38 (s, 1H), 8.17 (s, 1H), 8.03 (d, 1H), 8.01-7.96 (m, 2H), 7.87 (dd, 1H), 7.63-7.58 (m, 2H), 7.47-7.43 (m, 2H), 7.20-7.14 (m, 2H), 5.21-5.15 (m, 1H), 4.64 (t, 1H), 3.49-3.38 (m, 2H), 2.10-2.01 (m, 1H), 1.96-1.88 (m, 1H).

### Example 1-121

### N-[(1R)-1-(4-Fluorophenyl)-3-hydroxpropyl]-6-[3-methyl-4-(trifluoromethyl)phenyl]-4-oxo-4,5-dihydropyrazolo[1,5-a]pyrazine-2-carboxamide

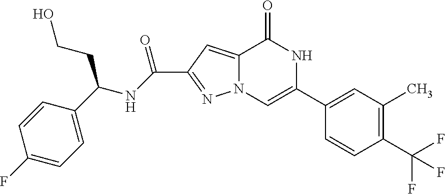 [SMILES: Cc1cc(-c2cn3nc(C(=O)N[C@H](CCO)c4ccc(F)cc4)cc3c(=O)[nH]2)ccc1C(F)(F)F]

The title compound was prepared according to general procedure 1-A starting from 6-[3-methyl-4-(trifluoromethyl)phenyl]-4-oxo-4,5-dihydropyrazolo[1,5-a]pyrazine-2-carboxylic acid (Intermediate 200A, 50.0 mg, 148 μmol), (3R)-3-amino-3-(4-fluorophenyl)propan-1-ol (30.1 mg, 178 μmol), HATU (67.6 mg, 178 μmol) and N,N-diisopropylethylamine (77 μl, 440 μmol) in DMF (1.3 ml). After cooling to RT, the reaction mixture was diluted with DMSO (2.0 ml) and purified by twofold preparative HPLC (first Method P2, then Method P7) to afford the title compound. Yield: 37.4 mg (51% of theory).

LC/MS [Method 3]: Rₜ=1.75 min; MS (ESIpos): m/z=489 [M+H]⁺.

¹H-NMR (500 MHz, DMSO-d₆): δ [ppm]=11.82 (s, 1H), 8.90 (d, 1H), 8.18 (s, 1H), 7.88 (s, 1H), 7.80-7.76 (m, 2H), 7.47-7.42 (m, 2H), 7.38 (s, 1H), 7.18-7.13 (m, 2H), 5.22-5.16 (m, 1H), 4.64 (t, 1H), 3.47-3.35 (m, 2H), 2.11-2.03 (m, 1H), 1.97-1.89 (m, 1H).

### Example 1-122

### 6-(3,4-Dimethylphenyl)-N-[(1R)-1-(4-fluorophenyl)-3-hydroxpropyl]-7-methyl-4-oxo-3-(trifluoromethyl)-4,5-dihydropyrazolo[1,5-a]pyrazine-2-carboxamide

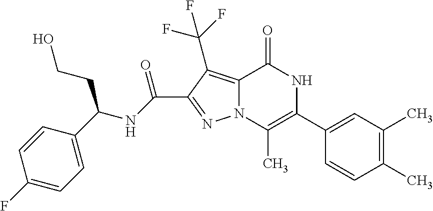 [SMILES: Cc1ccc(-c2[nH]c(=O)c3c(C(F)(F)F)c(C(=O)N[C@H](CCO)c4ccc(F)cc4)nn3c2C)cc1C]

The title compound was prepared according to general procedure 1-C starting from 6-(3,4-dimethylphenyl)-7-methyl-4-oxo-3-(trifluoromethyl)-4,5-dihydropyrazolo[1,5-a]pyrazine-2-carboxylic acid (Intermediate 151A, 100 mg, 274 μmol), (3R)-3-amino-3-(4-fluorophenyl)propan-1-ol (55.6 mg, 328 μmol), HATU (125 mg, 328 μmol) and N,N-diisopropylethylamine (140 μl, 820 μmol) in DMF (2.5 ml). After cooling to RT, the reaction mixture was diluted with DMSO (2.0 ml) and purified by preparative HPLC (Method P11) to afford the title compound. Yield: 67.2 mg (48% of theory).

LC/MS [Method 3]: Rₜ=1.85 min; MS (ESIpos): m/z=517 [M+H]⁺.

¹H-NMR (500 MHz, DMSO-d₆): δ [ppm]=11.86 (br. s, 1H), 9.18 (d, 1H), 7.44-7.38 (m, 2H), 7.31-7.27 (m, 2H), 7.24-7.21 (m, 1H), 7.21-7.16 (m, 2H), 5.17-5.12 (m, 1H), 4.58 (t, 1H), 3.48-3.34 (m, 2H), 2.34 (s, 3H), 2.32-2.27 (m, 6H), 2.02-1.95 (m, 1H), 1.89-1.82 (m, 1H).

### Example 1-123

### 6-(3,4-Dimethylphenyl)-N-[(1R)-1-(4-fluorophenyl)ethyl]-7-methyl-4-oxo-3-(trifluoromethyl)-4,5-dihydropyrazolo[1,5-a]pyrazine-2-carboxamide

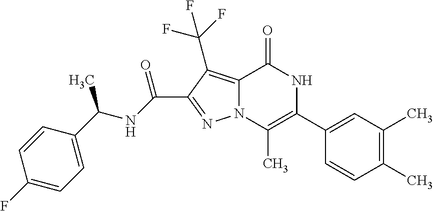 [SMILES: Cc1ccc(-c2[nH]c(=O)c3c(C(F)(F)F)c(C(=O)N[C@H](C)c4ccc(F)cc4)nn3c2C)cc1C]

The title compound was prepared according to general procedure 1-C starting from 6-(3,4-dimethylphenyl)-7-methyl-4-oxo-3-(trifluoromethyl)-4,5-dihydropyrazolo[1,5-a]pyrazine-2-carboxylic acid (Intermediate 151A, 100 mg, 274 μmol), (1R)-1-(4-fluorophenyl)ethan-1-amine (44 μl, 330 μmol), HATU (125 mg, 328 μmol) and N,N-diisopropylethylamine (140 μl, 820 μmol) in DMF (2.5 ml). After cooling to RT, the reaction mixture was diluted with DMSO (2.0 ml) and purified by preparative HPLC (Method P11) to afford the title compound. Yield: 65.5 mg (49% of theory).

LC/MS [Method 3]: Rₜ=2.14 min; MS (ESIpos): m/z=487 [M+H]⁺.

¹H-NMR (500 MHz, DMSO-d₆): δ [ppm]=11.86 (br. s, 1H), 9.20 (d, 1H), 7.46-7.41 (m, 2H), 7.31-7.26 (m, 2H), 7.26-7.21 (m, 1H), 7.21-7.16 (m, 2H), 5.19-5.12 (m, 1H), 2.35 (s, 3H), 2.31-2.27 (m, 6H), 1.45 (d, 3H).

### Example 1-124

### tert.-Butyl 3-(4-fluorophenyl)-3-{[6-(naphthalen-2-yl)-4-oxo-3-(trifluoromethyl)-4,5-dihydropyrazolo[1,5-a]pyrazine-2-carbonyl]amino}azetidine-1-carboxylate

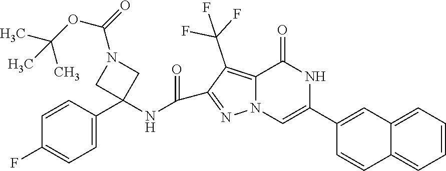 [SMILES: CC(C)(C)OC(=O)N1CC(NC(=O)c2nn3cc(-c4ccc5ccccc5c4)[nH]c(=O)c3c2C(F)(F)F)(c2ccc(F)cc2)C1]

The title compound was prepared according to general procedure 1-C starting from 6-(naphthalen-2-yl)-4-oxo-3-(trifluoromethyl)-4,5-dihydropyrazolo[1,5-a]pyrazine-2-carboxylic acid (Intermediate 35A, 701 mg, 1.88 mmol), tert.-butyl 3-amino-3-(4-fluorophenyl)azetidine-1-carboxylate (Intermediate 103A, 500 mg, 1.88 mmol), HATU (892 mg, 2.35 mmol) and N,N-diisopropylethylamine (820 μl, 4.7 mmol) in DMF (20 ml). The reaction mixture was stirred at RT for 6 days and then in portions directly purified by preparative HPLC (Method P5) to afford the title compound. Yield: 463 mg (40% of theory).

LC/MS [Method 3]: Rₜ=2.20 min; MS (ESIpos): m/z=566 [M+H−C₄H₈]⁺.

¹H-NMR (500 MHz, DMSO-d₆): δ [ppm]=12.28 (br. s, 1H), 9.98 (s, 1H), 8.42-8.38 (m, 2H), 8.06 (d, 1H), 8.02-7.97 (m, 2H), 7.90 (dd, 1H), 7.64-7.58 (m, 2H), 7.58-7.48 (m, 2H), 7.29-7.22 (m, 2H), 4.37-4.25 (m, 2H), 4.25-4.09 (m, 2H), 1.42 (s, 9H).

### Example 1-125

### N-[3-(4-Fluorophenyflazetidin-3-yl]-6-(naphthalen-2-yl)-4-oxo-3-(trifluoromethyl)-4,5-dihydropyrazolo[1,5-a]pyrazine-2-carboxamide hydrochloride

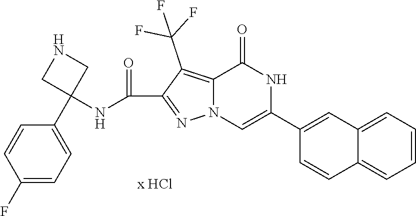 [SMILES: O=C(NC1(c2ccc(F)cc2)CNC1)c1nn2cc(-c3ccc4ccccc4c3)[nH]c(=O)c2c1C(F)(F)F]

A solution of ted.-butyl 3-(4-fluorophenyl)-3-{[6-(naphthalen-2-yl)-4-oxo-3-(trifluoromethyl)-4,5-dihydropyrazolo[1,5-a]pyrazine-2-carbonyl]amino}azetidine-1-carboxylate (Example 1-124, 460 mg, 740 μmol) in dioxane (2.0 ml) was treated with hydrogen chloride (2.0 ml, 4.0 M solution in dioxane, 8.0 mmol) at RT. The mixture was stirred overnight and then evaporated to afford the crude product which was used in the next step without further purification. Yield: 464 mg (quant.).

LC/MS [Method 4]: Rₜ=1.35 min; MS (ESIpos): m/z=522 [M+H]⁺.

¹H-NMR (500 MHz, DMSO-d₆): δ [ppm]=9.87 (s, 1H), 8.42 (s, 1H), 8.39 (s, 1H), 8.25 (s, 1H), 8.05 (d, 1H), 8.02-7.97 (m, 2H), 7.91 (dd, 1H), 7.67-7.54 (m, 4H), 7.29-7.20 (m, 2H), 4.16 (d, 2H), 3.93 (d, 2H).

### Example 1-126

### N-[1-Ethyl-3-(4-fluorophenyl)azetidin-3-yl]-6-(naphthalen-2-yl)-4-oxo-3-(trifluoromethyl)-4,5-dihydropyrazolo[1,5-a]pyrazine-2-carboxamide

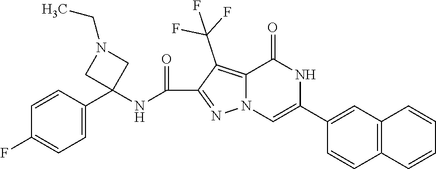 [SMILES: CCN1CC(NC(=O)c2nn3cc(-c4ccc5ccccc5c4)[nH]c(=O)c3c2C(F)(F)F)(c2ccc(F)cc2)C1]

A mixture of N-[3-(4-fluorophenyl)azetidin-3-yl]-6-(naphthalen-2-yl)-4-oxo-3-(trifluoromethyl)-4,5-dihydropyrazolo[1,5-a]pyrazine-2-carboxamide hydrochloride (Example 1-125, 50.0 mg, 89.6 μmol) and acetaldehyde (100 μl, 1.8 mmol) in acetic acid (1.0 ml) was stirred at RT for 30 min. Then, sodium cyanoborohydride (16.9 mg, 269 μmol) was added, and stirring was continued overnight. Methanol (1.0 ml) and hydrochloric acid (10 drops, 1.0 M solution) were added and after stirring for 5 min at RT, the mixture was directly purified by preparative HPLC (Method P11) to afford the title compound. Yield: 26.0 mg (53% of theory).

LC/MS [Method 3]: Rₜ=1.40 min; MS (ESIpos): m/z=550 [M+H]⁺.

¹H-NMR (500 MHz, DMSO-d₆): δ [ppm]=12.06 (br. s, 1H), 9.76 (s, 1H), 8.43-8.39 (m, 2H), 8.05 (d, 1H), 8.03-7.98 (m, 2H), 7.90 (dd, 1H), 7.67-7.59 (m, 4H), 7.24-7.19 (m, 2H), 3.66 (d, 2H), 3.39 (d, 2H), 0.93 (t, 3H).

### Example 1-127

### N-[3-(4-Fluorophenyl)-1-propylazetidin-3-yl]-6-(naphthalen-2-yl)-4-oxo-3-(trifluoromethyl)-4,5-dihydropyrazolo[1,5-a]pyrazine-2-carboxamide

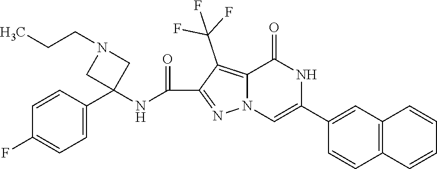 [SMILES: CCCN1CC(NC(=O)c2nn3cc(-c4ccc5ccccc5c4)[nH]c(=O)c3c2C(F)(F)F)(c2ccc(F)cc2)C1]

A mixture of N-[3-(4-fluorophenyl)azetidin-3-yl]-6-(naphthalen-2-yl)-4-oxo-3-(trifluoromethyl)-4,5-dihydropyrazolo[1,5-a]pyrazine-2-carboxamide hydrochloride (Example 1-125, 50.0 mg, 89.6 μmol) and propionaldehyde (130 μl, 1.8 mmol) in acetic acid (1.0 ml) was stirred at RT for 30 min. Then, sodium cyanoborohydride (16.9 mg, 269 μmol) was added, and stirring was continued overnight. Methanol (1.0 ml) and hydrochloric acid (10 drops, 1.0 M solution) were added and after stirring for 5 min at RT, the mixture was directly purified by preparative HPLC (Method P11) to afford the title compound. Yield: 35.0 mg (69% of theory).

LC/MS [Method 3]: Rₜ=1.44 min; MS (ESIpos): m/z=564 [M+H]⁺.

¹H-NMR (500 MHz, DMSO-d₆): δ [ppm]=12.26 (br. s, 1H), 9.76 (s, 1H), 8.41 (s, 2H), 8.05 (d, 1H), 8.03-7.97 (m, 2H), 7.90 (dd, 1H), 7.67-7.59 (m, 4H), 7.25-7.19 (m, 2H), 3.66 (d, 2H), 3.40 (d, 2H), 2.47-2.43 (m, 2H), 1.39-1.30 (m, 2H), 0.88 (t, 3H).

### Example 1-128

### N-[3-(4-Fluorophenyl)-1-(2-methylpropyl)azetidin-3-yl]-6-(naphthalen-2-yl)-4-oxo-3-(trifluoromethyl)-4,5-dihydropyrazolo[1,5-a]pyrazine-2-carboxamide

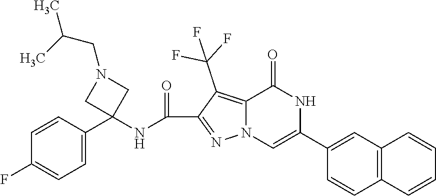 [SMILES: CC(C)CN1CC(NC(=O)c2nn3cc(-c4ccc5ccccc5c4)[nH]c(=O)c3c2C(F)(F)F)(c2ccc(F)cc2)C1]

A mixture of N-[3-(4-fluorophenyl)azetidin-3-yl]-6-(naphthalen-2-yl)-4-oxo-3-(trifluoromethyl)-4,5-dihydropyrazolo[1,5-a]pyrazine-2-carboxamide hydrochloride (Example 1-125, 50.0 mg, 89.6 μmol) and isobutyraldehyde (98 μl, 1.1 mmol) in acetic acid (1.0 ml) was stirred at RT for 30 min. Then, sodium cyanoborohydride (16.9 mg, 269 μmol) was added, and stirring was continued overnight. Methanol (1.0 ml) and hydrochloric acid (10 drops, 1.0 M solution) were added and after stirring for 5 min at RT, the mixture was directly purified by preparative HPLC (Method P11) to afford the title compound. Yield: 30.0 mg (56% of theory).

LC/MS [Method 3]: Rₜ=1.50 min; MS (ESIpos): m/z=578 [M+H]⁺.

¹H-NMR (500 MHz, DMSO-d₆): δ [ppm]=12.26 (br. s, 1H), 9.77 (s, 1H), 8.41 (s, 2H), 8.05 (d, 1H), 8.03-7.98 (m, 2H), 7.90 (dd, 1H), 7.69-7.64 (m, 2H), 7.64-7.60 (m, 2H), 7.25-7.20 (m, 2H), 3.66 (d, 2H), 3.42 (d, 2H), 2.33-2.29 (m, 2H), 1.63-1.55 (m, 1H), 0.88 (d, 6H).

### Example 1-129

### 6-(3-Chloro-4-methylphenyl)-N-(3,4-dimethoxybenzyl)-7-methyl-4-oxo-4,5-dihydropyrazolo-[1,5-a]pyrazine-2-carboxamide

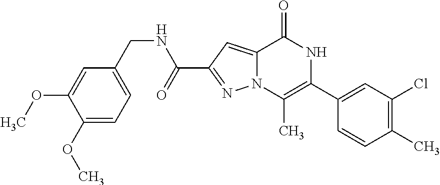

6-(3-Chloro-4-methylphenyl)-7-methyl-4-oxo-4,5-dihydropyrazolo[1,5-a]pyrazine-2-carboxylic acid (Intermediate 185A, 200 mg, 629 μmol) was dissolved in DMF (15.0 ml). N,N-Diisopropylethylamine (330 μl, 1.9 mmol), HATU (263 mg, 692 μmol) and 1-(3,4-dimethoxyphenyl)methanamine (105 mg, 629 μmol) were added, and the mixture was stirred at RT overnight. Then, the reaction mixture was partitioned between water and dichloromethane. The organic layer was recovered, and the aqueous phase was extracted with dichloromethane. The combined organic phases were washed with brine and dried over sodium sulfate. Removal of solvent under reduced pressure gave 650 mg of crude product which was dissolved in DMSO and purified by preparative HPLC (Method P13; gradient: A 70%/B 30% to A 30%/B 70% in 5 min, 150 ml/min, three runs). Combination and lyophilisation of the appropriate fractions gave the title compound. Yield: 263 mg (89% of theory).

LC/MS [Method 34]: Rₜ=1.18 min; (ESIpos): m/z=467 [M+H]⁺.

¹H-NMR (400 MHz, DMSO-d₆): δ [ppm]=11.56 (br. s, 1H), 8.93 (t, 1H), 7.47-7.61 (m, 2H), 7.36-7.42 (m, 2H), 6.97 (d, 1H), 6.90 (d, 1H), 6.86 (dd, 1H), 4.42 (d, 2H), 3.73 (s, 3H), 3.72 (s, 3H), 2.39 (s, 3H), 2.37 (s, 3H).

### Example 1-130

### N-[2-(3-Cyclopropyl-1H-pyrazol-1-yl)ethyl]-6-(3,4-dimethylphenyl)-7-methyl-4-oxo-3-(trifluoromethyl)-4,5-dihydropyrazolo[1,5-a]pyrazine-2-carboxamide

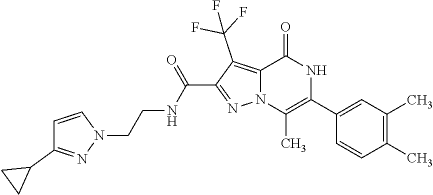 [SMILES: Cc1ccc(-c2[nH]c(=O)c3c(C(F)(F)F)c(C(=O)NCCn4ccc(C5CC5)n4)nn3c2C)cc1C]

The title compound was prepared according to general procedure 1-C starting from 6-(3,4-dimethylphenyl)-7-methyl-4-oxo-3-(trifluoromethyl)-4,5-dihydropyrazolo[1,5-a]pyrazine-2-carboxylic acid (Intermediate 151A, 100 mg, 274 μmol), 2-(3-cyclopropyl-1H-pyrazol-1-yl)ethan-1-amine (45.5 mg, 301 μmol), HATU (125 mg, 328 μmol) and N,N-diisopropylethylamine (140 μl, 820 μmol) in DMF (2.5 ml). The reaction mixture was diluted with DMSO (1.0 ml), and the resulting solution was directly purified by preparative HPLC (Method P11) to afford the title compound. Yield: 45.3 mg (32% of theory, 95% purity).

LC/MS [Method 3]: Rₜ=1.89 min; MS (ESIpos): m/z=499 [M+H]⁺.

¹H-NMR (500 MHz, DMSO-d₆): δ [ppm]=11.85 (s, 1H), 8.81 (t, 1H), 7.53 (d, 1H), 7.31-7.26 (m, 2H), 7.26-7.22 (m, 1H), 5.91 (d, 1H), 4.18 (t, 2H), 3.64-3.59 (m, 2H), 2.34 (s, 3H), 2.31-2.28 (m, 6H), 1.87-1.82 (m, 1H), 0.84-0.80 (m, 2H), 0.63-0.59 (m, 2H).

### Example 1-131

### 3-Cyclopropyl-N-[(1R)-1-(4-fluorophenyl)ethyl]-6-[3-methyl-4-(trifluoromethyl)phenyl]-4-oxo-4,5-dihydropyrazolo[1,5-a]pyrazine-2-carboxamide

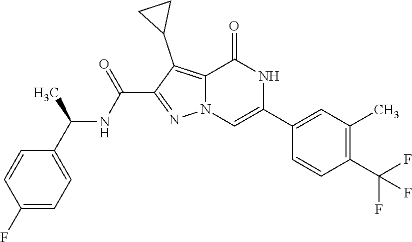 [SMILES: Cc1cc(-c2cn3nc(C(=O)N[C@H](C)c4ccc(F)cc4)c(C4CC4)c3c(=O)[nH]2)ccc1C(F)(F)F]

The title compound was prepared according to general procedure 1-C starting from 3-cyclopropyl-6-[3-methyl-4-(trifluoromethyl)phenyl]-4-oxo-4,5-dihydropyrazolo[1,5-a]pyrazine-2-carboxylic acid (Intermediate 191A, 70.0 mg, 186 μmol), (1R)-1-(4-fluorophenyl)ethan-1-amine (25.8 mg, 186 μmol), HATU (88.2 mg, 232 μmol) and N,N-diisopropylethylamine (81 μl, 460 μmol) in DMF (3.0 ml). The reaction mixture was directly purified by preparative HPLC (Method P5) to afford the title compound. Yield: 45.3 mg (32% of theory, 95% purity).

LC/MS [Method 3]: Rₜ=2.31 min; MS (ESIpos): m/z=499 [M+H]⁺.

¹H-NMR (500 MHz, DMSO-d₆): δ [ppm]=11.55 (br. s, 1H), 8.75 (d, 1H), 8.12 (s, 1H), 7.87 (s, 1H), 7.78-7.74 (m, 2H), 7.48-7.42 (m, 2H), 7.20-7.13 (m, 2H), 5.17-5.11 (m, 1H), 2.75-2.69 (m, 1H), 1.47 (d, 3H), 1.19-1.07 (m, 2H), 0.89-0.80 (m, 2H).

### Example 1-132

### 3-Cyclopropyl-N-[(1S)-1-(4-fluorophenyl)-2-hydroxy-2-methylpropyl]-6-[3-methyl-4-(trifluoromethyl)phenyl]-4-oxo-4,5-dihydropyrazolo[1,5-a]pyrazine-2-carboxamide

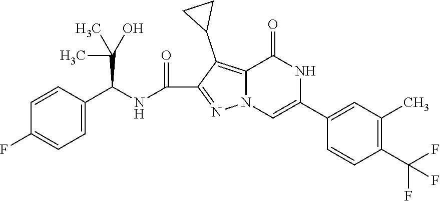

The title compound was prepared according to general procedure 1-C starting from 3-cyclopropyl-6-[3-methyl-4-(trifluoromethyl)phenyl]-4-oxo-4,5-dihydropyrazolo[1 ,5-a]pyrazine-2-carboxylic acid (Intermediate 191A, 70.0 mg, 186 μmol), (1S)-1-amino-1-(4-fluorophenyl)-2-methylpropan-2-ol (Intermediate 36A, 34.0 mg, 186 μmol), HATU (88.2 mg, 232 μmol) and N,N-diisopropylethylamine (81 μl, 460 μmol) in DMF (3.0 ml). The reaction mixture was directly purified by preparative HPLC (Method P5) to afford the title compound. Yield: 33.0 mg (31% of theory, 96% purity).

LC/MS [Method 3]: Rₜ=2.18 min; MS (ESIpos): m/z=543 [M+H]⁺.

¹H-NMR (500 MHz, DMSO-d₆): δ [ppm]=11.55 (br. s, 1H), 8.35 (d, 1H), 8.26 (s, 1H), 7.92 (s, 1H), 7.83-7.69 (m, 2H), 7.47-7.39 (m, 2H), 7.17-7.10 (m, 2H), 4.91 (s, 1H), 4.84 (d, 1H), 2.86-2.80 (m, 1H), 1.25 (s, 3H), 1.23-1.13 (m, 2H), 0.98 (s, 3H), 0.90-0.78 (m, 2H).

### Example 1-133

### N-[(1S)-1-(4-Fluorophenyl)-2-hydroxy-2-methylpropyl]-4-oxo-6-(5,6,7,8-tetrahydronaphthalen-2-yl)-4,5-dihydropyrazolo[1,5-a]pyrazine-2-carboxamide

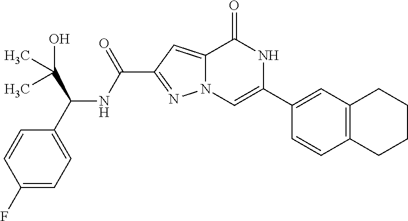 [SMILES: CC(C)(O)[C@@H](NC(=O)c1cc2c(=O)[nH]c(-c3ccc4c(c3)CCCC4)cn2n1)c1ccc(F)cc1]

The title compound was prepared according to general procedure 1-C starting from 4-oxo-6-(5,6,7,8-tetrahydronaphthalen-2-yl)-4,5-dihydropyrazolo[1,5-a]pyrazine-2-carboxylic acid (Intermediate 136A, 57.0 mg, 184 μmol), (1S)-1-amino-1-(4-fluorophenyl)-2-methylpropan-2-ol (Intermediate 36A, 37.1 mg, 203 μmol), HATU (87.6 mg, 230 μmol) and N,N-diisopropylethylamine (80 μl, 460 μmol) in DMF (2.3 ml). The reaction mixture was diluted with DMSO (2.0 ml), and the resulting solution was directly purified by preparative HPLC (Method P11) to afford the title compound. Yield: 13.1 mg (14% of theory, 93% purity).

LC/MS [Method 3]: Rₜ=1.98 min; MS (ESIpos): m/z=475 [M+H]⁺.

¹H-NMR (500 MHz, DMSO-d₆): δ [ppm]=11.66 (br. s, 1H), 8.29 (d, 1H), 8.12 (s, 1H), 7.52-7.41 (m, 4H), 7.29 (s, 1H), 7.18-7.09 (m, 3H), 5.00 (s, 1H), 4.87 (d, 1H), 2.81-2.74 (m, 4H), 1.80-1.72 (m, 4H), 1.26 (s, 3H), 0.97 (s, 3H).

### Example 1-134

### 6-(2,3-Dihydro-1H-inden-5-yl)-N-[(1S)-1-(4-fluorophenyl)-2-hydroxy-2-methylpropyl]-4-oxo-4,5-dihydropyrazolo[1,5-a]pyrazine-2-carboxamide

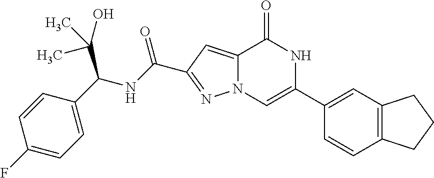 [SMILES: CC(C)(O)[C@@H](NC(=O)c1cc2c(=O)[nH]c(-c3ccc4c(c3)CCC4)cn2n1)c1ccc(F)cc1]

The title compound was prepared according to general procedure 1-C starting from 6-(2,3-dihydro-1H-inden-5-yl)-4-oxo-4,5-dihydropyrazolo[1,5-a]pyrazine-2-carboxylic acid (Intermediate 132A, 100 mg, 339 μmol), (1S)-1-amino-1-(4-fluorophenyl)-2-methylpropan-2-ol (Intermediate 36A, 68.3 mg, 373 μmol), HATU (161 mg, 423 μmol) and N,N-diisopropylethylamine (150 μl, 850 μmol) in DMF (2.8 ml). The reaction mixture was diluted with DMSO (2.0 ml), and the resulting solution was directly purified by preparative HPLC (Method P11) to afford the title compound. Yield: 17.0 mg (10% of theory, 95% purity).

LC/MS [Method 3]: Rₜ=1.88 min; MS (ESIpos): m/z=461 [M+H]⁺.

¹H-NMR (500 MHz, DMSO-d₆): δ [ppm]=11.67 (br. s, 1H), 8.29 (d, 1H), 8.10 (s, 1H), 7.62 (s, 1H), 7.51 (d, 1H), 7.48-7.41 (m, 2H), 7.36-7.25 (m, 2H), 7.17-7.08 (m, 2H), 5.00 (br. s, 1H), 4.87 (d, 1H), 2.96-2.85 (m, 4H), 2.10-2.01 (m, 2H), 1.26 (s, 3H), 0.97 (s, 3H).

### Example 1-135

### 6-(3,4-Dimethylphenyl)-N-[(1S)-1-(4-fluorophenyl)-2-hydroxy-2-methylpropyl]-4-oxo-3-(propan-2-yl)-4,5-dihydropyrazolo[1,5-a]pyrazine-2-carboxamide

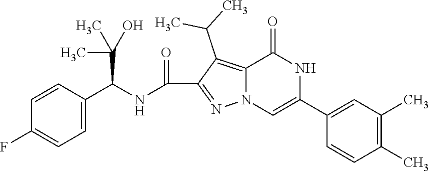 [SMILES: Cc1ccc(-c2cn3nc(C(=O)N[C@@H](c4ccc(F)cc4)C(C)(C)O)c(C(C)C)c3c(=O)[nH]2)cc1C]

The title compound was prepared according to general procedure 1-C starting from 6-(3,4-dimethylphenyl)-4-oxo-3-(propan-2-yl)-4,5-dihydropyrazolo[1,5-a]pyrazine-2-carboxylic acid (Intermediate 128A, 100 mg, 307 μmol), (1S)-1-amino-1-(4-fluorophenyl)-2-methylpropan-2-ol (Intermediate 36A, 61.9 mg, 338 μmol), HATU (146 mg, 384 μmol) and N,N-diisopropylethylamine (130 μl, 770 μmol) in DMF (2.5 ml). The reaction mixture was diluted with DMSO (2.0 ml), and the resulting solution was directly purified by preparative HPLC (Method P11) to afford the title compound. Yield: 79.0 mg (50% of theory, 95% purity).

LC/MS [Method 3]: Rₜ=2.16 min; MS (ESIpos): m/z=491 [M+H]⁺.

¹H-NMR (500 MHz, DMSO-d₆): δ [ppm]=11.43 (br. s, 1H), 8.32 (d, 1H), 8.05 (s, 1H), 7.60 (s, 1H), 7.50 (dd, 1H), 7.46-7.41 (m, 2H), 7.24 (d, 1H), 7.17-7.10 (m, 2H), 4.92 (s, 1H), 4.85 (d, 1H), 4.18-4.10 (m, 1H), 2.28 (s, 3H), 2.27 (s, 3H), 1.33-1.28 (m, 6H), 1.25 (s, 3H), 0.98 (s, 3H).

### Example 1-136

### N-[1-(4-Hydroxphenyl)ethyl]-6-(2-naphthyl)-4-oxo-5H-pyrazolo[1,5-a]pyrazine-2-carboxamide

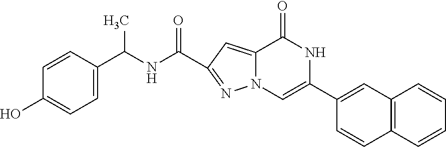 [SMILES: CC(NC(=O)c1cc2c(=O)[nH]c(-c3ccc4ccccc4c3)cn2n1)c1ccc(O)cc1]

The title compound was prepared according to general procedure 1-C starting from 6-(naphthalen-2-yl)-4-oxo-4,5-dihydropyrazolo[1,5-a]pyrazine-2-carboxylic acid (Intermediate 3A, 100 mg, 328 μmol), 4-(1-aminoethyl)phenol (49.4 mg, 360 μmol), HATU (156 mg, 409 μmol) and N,N-diisopropylethylamine (170 μl, 980 μmol) in DMF (2.5 ml). The reaction mixture was diluted with DMSO (2.0 ml), and the resulting solution was directly purified by preparative HPLC (Method P11) to afford the title compound. Yield: 48.3 mg (34% of theory, 97% purity).

LC/MS [Method 3]: Rₜ=1.62 min; MS (ESIpos): m/z=425 [M+H]⁺.

¹H-NMR (500 MHz, DMSO-d₆): δ [ppm]=11.86 (br. s, 1H), 9.29 (br. s, 1H), 8.58 (d, 1H), 8.38 (d, 1H), 8.18 (s, 1H), 8.03 (d, 1H), 8.01-7.96 (m, 2H), 7.88 (dd, 1H), 7.62-7.57 (m, 2H), 7.40 (s, 1H), 7.25-7.20 (m, 2H), 6.74-6.70 (m, 2H), 5.13-5.07 (m, 1H), 1.47 (d, 3H).

### Example 1-137

### 6-(3,4-Dimethylphenyl)-N-[1-(4-hydroxphenyl)ethyl]-4-oxo-5H-pyrazolo[1,5-a]pyrazine-2-carboxamide

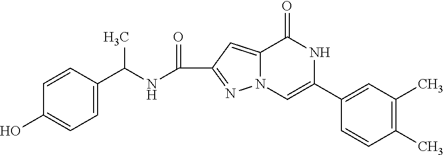 [SMILES: Cc1ccc(-c2cn3nc(C(=O)NC(C)c4ccc(O)cc4)cc3c(=O)[nH]2)cc1C]

The title compound was prepared according to general procedure 1-C starting from 6-(3,4-dimethylphenyl)-4-oxo-4,5-dihydropyrazolo[1,5-a]pyrazine-2-carboxylic acid (Intermediate 23A, 100 mg, 353 μmol), 4-(1-aminoethyl)phenol (53.3 mg, 388 μmol), HATU (168 mg, 441 μmol) and N,N-diisopropylethylamine (180 μl, 1.1 mmol) in DMF (2.5 ml). The reaction mixture was diluted with DMSO (2.0 ml), and the resulting solution was directly purified by preparative HPLC (Method P11) to afford the title compound. Yield: 37.9 mg (27% of theory).

LC/MS [Method 3]: Rₜ=1.60 min; MS (ESIpos): m/z=403 [M+H]⁺.

¹H-NMR (500 MHz, DMSO-d₆): δ [ppm]=11.64 (br. s, 1H), 9.26 (s, 1H), 8.55 (d, 1H), 7.95 (s, 1H), 7.57-7.53 (m, 1H), 7.49-7.44 (m, 1H), 7.36 (s, 1H), 7.25 (d, 1H), 7.23-7.18 (m, 2H), 6.73-6.69 (m, 2H), 5.12-5.05 (m, 1H), 2.28 (s, 3H), 2.27 (s, 3H), 1.46 (d, 3H).

### Example 1-138

### N-[(2R)-Butan-2-yl]-3-cyclopropyl-6-[3-methyl-4-(trifluoromethyl)phenyl]-4-oxo-4,5-dihydropyrazolo[1,5-a]pyrazine-2-carboxamide

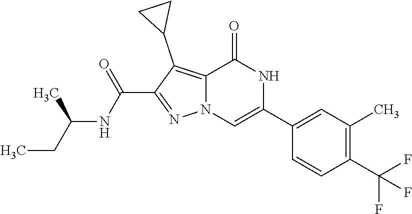 [SMILES: CC[C@@H](C)NC(=O)c1nn2cc(-c3ccc(C(F)(F)F)c(C)c3)[nH]c(=O)c2c1C1CC1]

The title compound was prepared according to general procedure 1-C starting from 3-cyclopropyl-6-[3-methyl-4-(trifluoromethyl)phenyl]-4-oxo-4,5-dihydropyrazolo[1,5-a]pyrazine-2-carboxylic acid (Intermediate 191A, 70.0 mg, 186 μmol), (2R)-butan-2-amine (13.6 mg, 186 μmol), HATU (88.2 mg, 232 μmol) and N,N-diisopropylethylamine (81 μl, 460 μmol) in DMF (3.0 ml). The reaction mixture was directly purified by preparative HPLC (Method P5) to afford the title compound. Yield: 30.0 mg (37% of theory).

LC/MS [Method 7]: Rₜ=1.14 min; MS (ESIpos): m/z=433 [M+H]⁺.

¹H-NMR (500 MHz, DMSO-d₆): δ [ppm]=11.53 (br. s, 1H), 8.11 (s, 1H), 8.03 (d, 1H), 7.87 (s, 1H), 7.78-7.73 (m, 2H), 3.94-3.86 (m, 1H), 2.79-2.73 (m, 1H), 1.58-1.44 (m, 2H), 1.23-1.16 (m, 2H), 1.14 (d, 3H), 0.92-0.85 (m, 5H).

### Example 1-139

### 3-Cyclopropyl-N-[(2R)-3-methylbutan-2-yl]-6-[3-methyl-4-(trifluoromethyl)phenyl]-4-oxo-4,5-dihydropyrazolo[1,5-a]pyrazine-2-carboxamide

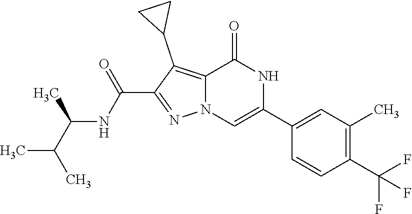 [SMILES: Cc1cc(-c2cn3nc(C(=O)N[C@H](C)C(C)C)c(C4CC4)c3c(=O)[nH]2)ccc1C(F)(F)F]

The title compound was prepared according to general procedure 1-C starting from 3-cyclopropyl-6-[3-methyl-4-(trifluoromethyl)phenyl]-4-oxo-4,5-dihydropyrazolo[1 ,5-a]pyrazine-2-carboxylic acid (Intermediate 191A, 70.0 mg, 186 μmol), (2R)-3-methylbutan-2-amine (16.2 mg, 186 μmol), HATU (88.2 mg, 232 μmol) and N,N-diisopropylethylamine (81 μl, 460 μmol) in DMF (3.0 ml). The reaction mixture was directly purified by preparative HPLC (Method P5) to afford the title compound. Yield: 31.0 mg (37% of theory).

LC/MS [Method 3]: Rₜ=2.33 min; MS (ESIpos): m/z=447 [M+H]⁺.

¹H-NMR (500 MHz, DMSO-d₆): δ [ppm]=11.52 (s, 1H), 8.14 (s, 1H), 8.00 (d, 1H), 7.88 (s, 1H), 7.79-7.73 (m, 2H), 3.85-3.78 (m, 1H), 2.78-2.72 (m, 1H), 1.80-1.73 (m, 1H), 1.21-1.14 (m, 2H), 1.10 (d, 3H), 0.92-0.87 (m, 8H).

### Example 1-140

### N-[(2R)-Butan-2-yl]-6-(3,4-dimethylphenyl)-4-oxo-3-(propan-2-yl)-4,5-dihydropyrazolo[1,5-a]-pyrazine-2-carboxamide

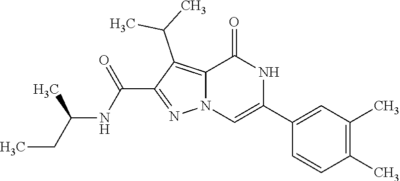 [SMILES: CC[C@@H](C)NC(=O)c1nn2cc(-c3ccc(C)c(C)c3)[nH]c(=O)c2c1C(C)C]

The title compound was prepared according to general procedure 1-C starting from 6-(3,4-di-methylphenyl)-4-oxo-3-(propan-2-yl)-4,5-dihydropyrazolo[1,5-a]pyrazine-2-carboxylic acid (Intermediate 128A, 70.0 mg, 215 μmol), (2R)-butan-2-amine (15.7 mg, 215 μmol), HATU (102 mg, 269 μmol) and N,N-diisopropylethylamine (94 μl, 540 μmol) in DMF (3.5 ml). The reaction mixture was directly purified by preparative HPLC (Method P5) to afford the title compound. Yield: 39.0 mg (48% of theory).

LC/MS [Method 7]: Rₜ=1.21 min; MS (ESIpos): m/z=381 [M+H]⁺.

¹H-NMR (500 MHz, DMSO-d₆): δ [ppm]=11.40 (br. s, 1H), 7.95 (d, 1H), 7.88 (s, 1H), 7.55 (s, 1H), 7.46 (dd, 1H), 7.24 (d, 1H), 4.11-4.04 (m, 1H), 3.95-3.87 (m, 1H), 2.28 (s, 3H), 2.27 (s, 3H), 1.58-1.45 (m, 2H), 1.34 (d, 6H), 1.14 (d, 3H), 0.88 (t, 3H).

### Example 1-141

### 6-(3,4-Dimethylphenyl)-N-[(2R)-3-methylbutan-2-yl]-4-oxo-3-(propan-2-yl)-4,5-dihydropyrazolo-[1,5-a]pyrazine-2-carboxamide

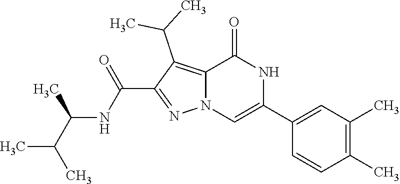 [SMILES: Cc1ccc(-c2cn3nc(C(=O)N[C@H](C)C(C)C)c(C(C)C)c3c(=O)[nH]2)cc1C]

The title compound was prepared according to general procedure 1-C starting from 6-(3,4-dimethylphenyl)-4-oxo-3-(propan-2-yl)-4,5-dihydropyrazolo[1,5-a]pyrazine-2-carboxylic acid (Intermediate 128A, 70.0 mg, 215 μmol), (2R)-3-methylbutan-2-amine (18.8 mg, 215 μmol), HATU (102 mg, 269 μmol) and N,N-diisopropylethylamine (94 μl, 540 μmol) in DMF (3.5 ml). The reaction mixture was directly purified by preparative HPLC (Method P5) to afford the title compound. Yield: 51.0 mg (60% of theory).

LC/MS [Method 3]: Rₜ=2.37 min; MS (ESIpos): m/z=395 [M+H]⁺.

¹H-NMR (500 MHz, DMSO-d₆): δ [ppm]=11.39 (s, 1H), 7.95-7.87 (m, 2H), 7.56 (s, 1H), 7.47 (dd, 1H), 7.23 (d, 1H), 4.10-4.02 (m, 1H), 3.86-3.79 (m, 1H), 2.28 (s, 3H), 2.26 (s, 3H), 1.81-1.73 (m, 1H), 1.34 (d, 6H), 1.11 (d, 3H), 0.90 (d, 6H).

### Example 1-142

### N-[(2R)-Butan-2-yl]-3-cyclopropyl-6-(3,4-dimethylphenyl)-4-oxo-4,5-dihydropyrazolo[1,5-a]-pyrazine-2-carboxamide

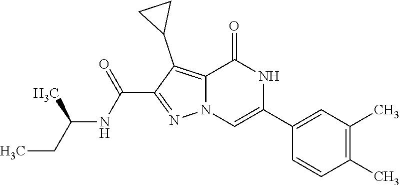 [SMILES: CC[C@@H](C)NC(=O)c1nn2cc(-c3ccc(C)c(C)c3)[nH]c(=O)c2c1C1CC1]

The title compound was prepared according to general procedure 1-C starting from 3-cyclopropyl-6-(3,4-dimethylphenyl)-4-oxo-4,5-dihydropyrazolo[1,5-a]pyrazine-2-carboxylic acid (Intermediate 92A, 70.0 mg, 216 μmol), (2R)-butan-2-amine (15.8 mg, 216 μmol), HATU (103 mg, 271 μmol) and N,N-diisopropylethylamine (94 μl, 540 μmol) in DMF (3.5 ml). The reaction mixture was directly purified by preparative HPLC (Method P5) to afford the title compound. Yield: 48.0 mg (59% of theory).

LC/MS [Method 3]: Rₜ=2.13 min; MS (ESIpos): m/z=379 [M+H]⁺.

¹H-NMR (500 MHz, DMSO-d₆): δ [ppm]=11.34 (s, 1H), 7.98 (d, 1H), 7.87 (s, 1H), 7.54 (s, 1H), 7.45 (dd, 1H), 7.23 (d, 1H), 3.94-3.86 (m, 1H), 2.81-2.75 (m, 1H), 2.27 (s, 3H), 2.26 (s, 3H), 1.56-1.45 (m, 2H), 1.21-1.16 (m, 2H), 1.14 (d, 3H), 0.91-0.84 (m, 5H).

### Example 1-143

### 3-Cyclopropyl-6-(3,4-dimethylphenyl)-N-[(2R)-3-methylbutan-2-yl]-4-oxo-4,5-dihydropyrazolo-[1,5-a]pyrazine-2-carboxamide

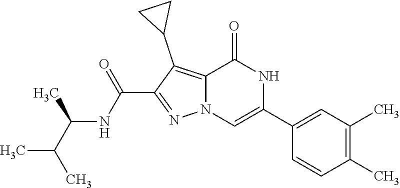 [SMILES: Cc1ccc(-c2cn3nc(C(=O)N[C@H](C)C(C)C)c(C4CC4)c3c(=O)[nH]2)cc1C]

The title compound was prepared according to general procedure 1-C starting from 3-cyclopropyl-6-(3,4-dimethylphenyl)-4-oxo-4,5-dihydropyrazolo[1,5-a]pyrazine-2-carboxylic acid (Intermediate 92A, 70.0 mg, 216 μmol), (2R)-3-methylbutan-2-amine (18.9 mg, 216 μmol), HATU (103 mg, 271 μmol) and N,N-diisopropylethylamine (94 μl, 540 μmol) in DMF (3.5 ml). The reaction mixture was directly purified by preparative HPLC (Method P5) to afford the title compound. Yield: 36.0 mg (42% of theory).

LC/MS [Method 3]: Rₜ=2.27 min; MS (ESIpos): m/z=393 [M+H]⁺.

¹H-NMR (500 MHz, DMSO-d₆): δ [ppm]=11.34 (s, 1H), 7.95 (d, 1H), 7.89 (s, 1H), 7.55 (s, 1H), 7.46 (dd, 1H), 7.23 (d, 1H), 3.85-3.77 (m, 1H), 2.80-2.74 (m, 1H), 2.27 (s, 3H), 2.26 (s, 3H), 1.80-1.72 (m, 1H), 1.24-1.14 (m, 2H), 1.10 (d, 3H), 0.92-0.88 (m, 6H), 0.88-0.84 (m, 2H).

### Example 1-144

### 3-Cyclopropyl-N-[(1R)-1-(4-fluorophenyl)-3-hydroxypropyl]-6-(naphthalen-2-yl)-4-oxo-4,5-dihydropyrazolo[1,5-a]pyrazine-2-carboxamide

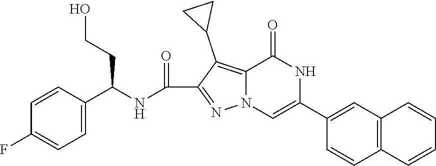 [SMILES: O=C(N[C@H](CCO)c1ccc(F)cc1)c1nn2cc(-c3ccc4ccccc4c3)[nH]c(=O)c2c1C1CC1]

The title compound was prepared according to general procedure 1-B starting from 3-cyclopropyl-6-(naphthalen-2-yl)-4-oxo-4,5-dihydropyrazolo[1,5-a]pyrazine-2-carboxylic acid (Intermediate 82A, 75.0 mg, 217 μmol), (3R)-3-amino-3-(4-fluorophenyl)propan-1-ol (40.4 mg, 239 μmol), CDI (38.7 mg, 239 μmol) and DMAP (13.3 mg, 109 μmol) in N-methylpyrrolidone (1.8 ml). After cooling to RT, the mixture was diluted with DMSO (5.0 ml), and the resulting solution was directly purified by preparative HPLC (Method P5) to afford the title compound. Yield: 22.0 mg (19% of theory).

LC/MS [Method 3]: Rₜ=1.42 min; MS (ESIpos): m/z=497 [M+H]⁺.

¹H-NMR (400 MHz, DMSO-d₆): δ [ppm]=8.39 (s, 1H), 8.24 (s, 1H), 8.02 (d, 1H), 8.00-7.94 (m, 2H), 7.88 (dd, 1H), 7.64-7.56 (m, 2H), 7.52-7.35 (m, 2H), 7.24-7.12 (m, 2H), 4.38-4.28 (m, 1H), 4.24-4.14 (m, 1H), 4.09 (t, 1H), 2.72-2.60 (m, 1H), 2.19-1.98 (m, 2H), 1.35-1.12 (m, 2H), 1.01-0.92 (m, 2H).

### Example 1-145

### N-[3-(4-Fluorophenyl)-1-ethylazetidin-3-yl]-6-(naphthalen-2-yl)-4-oxo-4,5-dihydropyrazolo[1,5-a]-pyrazine-2-carboxamide formic acid salt

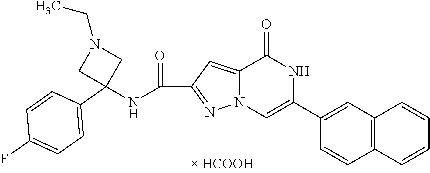 [SMILES: CCN1CC(NC(=O)c2cc3c(=O)[nH]c(-c4ccc5ccccc5c4)cn3n2)(c2ccc(F)cc2)C1]

Acetaldehyde (5.4 mg, 122 μmol) was added to a mixture of N-[3-(4-fluorophenyl)azetidin-3-yl]-6-(naphthalen-2-yl)-4-oxo-4,5-dihydropyrazolo[1,5-a]pyrazine-2-carboxamide hydrochloride (Example 1-48, 60.0 mg, 122 μmol) and acetic acid (70 μl, 1.2 mmol) in dichloromethane (10.0 ml), and the mixture was stirred at RT. After 1 h, sodium triacetoxyborohydride (38.9 mg, 184 μmol) was added, and stirring at RT was continued overnight. Water was added, and the mixture was neutralized with aqueous sodium bicarbonate solution. The layers were separated, and the aqueous layer was extracted with dichloromethane. The combined organic layers were dried over magnesium sulfate, filtered and concentrated under reduced pressure. The residue was purified by preparative HPLC (Method P1). After evaporation of the product-containing fractions, the residue was treated with isopropanol and evaporated again to afford the title compound (12.0 mg, 19% of theory).

LC/MS [Method 7]: Rₜ=0.74 min; MS (ESIpos): m/z=482 [M+H]⁺.

¹H-NMR (400 MHz, DMSO-d₆): δ [ppm]=11.8 (br. s, 1H), 9.45 (s, 1H), 8.38 (s, 1H), 8.15 (s, 1H), 8.06-7.96 (m, 3H), 7.88 (dd, 1H), 7.67-7.57 (m, 4H), 7.38 (s, 1H), 7.19-7.14 (m, 2H), 3.72 (br. d, 2H), 3.48 (br. d, 2H), 2.33 (s, 3H), 0.90 (t, 3H) [spectrum shows additional signals of residual isopropanol and minor unidentified impurities].

### Example 1-146

### N-[1-Acetyl-3-(4-fluorophenyl)azetidin-3-yl]-6-(2-naphthyl)-4-oxo-5H-pyrazolo[1,5-a]pyrazine-2-carboxamide

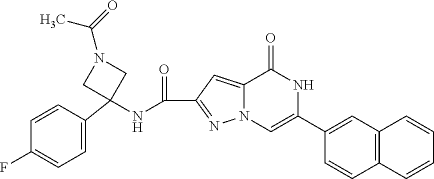 [SMILES: CC(=O)N1CC(NC(=O)c2cc3c(=O)[nH]c(-c4ccc5ccccc5c4)cn3n2)(c2ccc(F)cc2)C1]

Acetylchloride (14.1 mg, 180 μmol) was added to a mixture of N-[3-(4-fluorophenyl)azetidin-3-yl]-6-(naphthalen-2-yl)-4-oxo-4,5-dihydropyrazolo[1,5-a]pyrazine-2-carboxamide hydrochloride (Example 1-48, 200 mg, 163 μmol) and N,N-diisopropylethylamine (114 μl, 653 μmol) in dichloromethane (9.3 ml). The mixture was stirred at RT overnight. After aqueous work-up, drying of the organic phase over magnesium sulfate and evaporation, the crude product was purified by preparative HPLC (Method P1). After evaporation of the product-containing fractions, 20.5 mg (38% of theory) of the title compound were obtained.

LC/MS [Method 3]: Rₜ=1.61 min; MS (ESIpos): m/z=496 [M+H]⁺.

¹H-NMR (400 MHz, DMSO-d₆): δ [ppm]=11.9 (br. s, 1H), 9.66 (s, 1H), 8.38 (s, 1H), 8.16 (s, 1H), 8.06-7.95 (m, 3H), 7.89 (dd, 1H), 7.68-7.56 (m, 4H), 7.41 (s, 1H), 7.25-7.13 (m, 2H), 4.65-4.55 (m, 2H), 4.37 (d, 1H), 4.09 (d, 1H), 1.86 (s, 3H).

### Example 1-147

### 6-(3-Fluoro-4-methylphenyl)-N-[(15)-1-(4-fluorophenyl)-2-hydroxy-2-methylpropyl]-4-oxo-3-(trifluoromethyl)-4,5-dihydropyrazolo[1,5-a]pyrazine-2-carboxamide

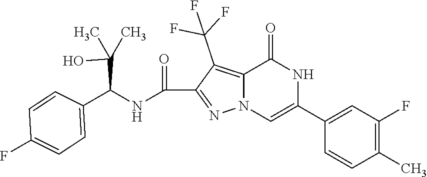 [SMILES: Cc1ccc(-c2cn3nc(C(=O)N[C@@H](c4ccc(F)cc4)C(C)(C)O)c(C(F)(F)F)c3c(=O)[nH]2)cc1F]

The title compound was prepared according to general procedure 1-C starting from 6-(3-fluoro-4-methylphenyl)-4-oxo-3-(trifluoromethyl)-4,5-dihydropyrazolo[1,5-a]pyrazine-2-carboxylic acid (Intermediate 215A, 100 mg, 88% purity, 248 μmol), (1S)-1-amino-1-(4-fluorophenyl)-2-methylpropan-2-ol (Intermediate 36A, 45.4 mg, 248 μmol), HATU (310 μmol) and N,N-diisopropylethylamine (130 μl, 740 μmol) in DMF (2.5 ml, 32 mmol). The reaction mixture was diluted with DMSO (2.0 ml) and directly purified by preparative HPLC (Method P14) to afford the title compound. Yield: 42.0 mg (33% of theory).

LC/MS [Method 3]: Rₜ=1.84 min; MS (ESIpos): m/z=521 [M+H]⁺.

¹H-NMR (400 MHz, DMSO-d₆): δ [ppm]=12.08 (s, 1H), 8.91 (d, 1H), 8.35 (s, 1H), 7.64 (d, 1H), 7.56 (d, 1H), 7.47-7.39 (m, 3H), 7.17-7.11 (m, 2H), 4.93 (d, 1H), 4.65 (br s, 1H), 2.29 (s, 3H), 1.18 (s, 3H), 1.04 (s, 3H).

### Example 1-148

### 6-(3-Chloro-4-methylphenyl)-N-[(1S)-1-(4-fluorophenyl)-2-hydroxy-2-methylpropyl]-3-methyl-4-oxo-4,5-dihydropyrazolo[1,5-a]pyrazine-2-carboxamide

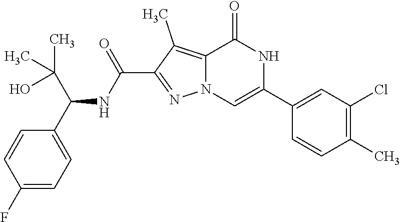 [SMILES: Cc1ccc(-c2cn3nc(C(=O)N[C@@H](c4ccc(F)cc4)C(C)(C)O)c(C)c3c(=O)[nH]2)cc1Cl]

The title compound was prepared according to general procedure 1-C starting from 6-(3-chloro-4-methylphenyl)-3-methyl-4-oxo-4,5-dihydropyrazolo[1,5-a]pyrazine-2-carboxylic acid (Intermediate 218A, 70.0 mg, 220 μmol), (1S)-1-amino-1-(4-fluorophenyl)-2-methylpropan-2-ol (Intermediate 36A, 44.4 mg, 242 μmol), HATU (101 mg, 264 μmol) and N,N-diisopropylethylamine (120 μl, 660 μmol) in DMF (5.0 ml). The reaction mixture was directly purified by preparative HPLC (Method P14) to afford the title compound. Yield: 17.8 mg (17% of theory).

LC/MS [Method 3]: Rₜ=2.02 min; MS (ESIpos): m/z=483 [M+H]⁺.

¹H-NMR (500 MHz, DMSO-d₆): δ [ppm]=11.53 (br s, 1H), 8.22 (d, 1H), 8.19 (s, 1H), 7.87 (d, 1H), 7.66 (dd, 1H), 7.46 (d, 1H), 7.45-7.36 (m, 2H), 7.17-7.09 (m, 2H), 4.99 (s, 1H), 4.82 (d, 1H), 2.59 (s, 3H), 2.38 (s, 3H), 1.26 (s, 3H), 0.96 (s, 3H).

### Example 1-149

### N-[(2R)-Butan-2-yl]-6-(3-chloro-4-methylphenyl)-3-methyl-4-oxo-4,5-dihydropyrazolo[1,5-a]-pyrazine-2-carboxamide

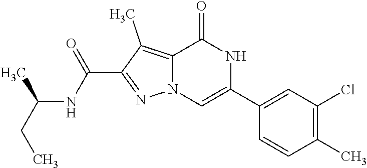 [SMILES: CC[C@@H](C)NC(=O)c1nn2cc(-c3ccc(C)c(Cl)c3)[nH]c(=O)c2c1C]

The title compound was prepared according to general procedure 1-C starting from 6-(3-chloro-4-methylphenyl)-3-methyl-4-oxo-4,5-dihydropyrazolo[1,5-a]pyrazine-2-carboxylic acid (Intermediate 218A, 70.0 mg, 220 μmol), (2R)-butan-2-amine (17.7 mg, 242 μmol), HATU (101 mg, 264 μmol) and N,N-diisopropylethylamine (120 μl, 660 μmol) in DMF (2.5 ml). The reaction mixture was directly purified by preparative HPLC (Method P14) to afford the title compound. Yield: 19.0 mg (22% of theory).

LC/MS [Method 7]: Rₜ=1.07 min; MS (ESIneg): m/z=371 [M−H]⁻.

¹H-NMR (400 MHz, DMSO-d₆): δ [ppm]=11.51 (s, 1H), 7.97 (d, 1H), 7.87-7.81 (m, 2H), 7.62 (dd, 1H), 7.46 (d, 1H), 3.98-3.85 (m, 1H), 2.61 (s, 3H), 2.38 (s, 3H), 1.61-1.43 (m, 2H), 1.15 (d, 3H), 0.87 (t, 3H).

### Example 1-150

### 6-(3-Chloro-4-methylphenyl)-N-[3-(4-fluorophenyl)-1-methylazetidin-3-yl]-3-methyl-4-oxo-4,5-dihydropyrazolo[1,5-a]pyrazine-2-carboxamide

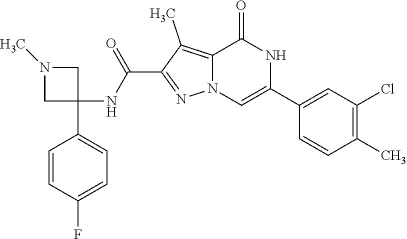 [SMILES: Cc1ccc(-c2cn3nc(C(=O)NC4(c5ccc(F)cc5)CN(C)C4)c(C)c3c(=O)[nH]2)cc1Cl]

The title compound was prepared according to general procedure 1-C starting from 6-(3-chloro-4-methylphenyl)-3-methyl-4-oxo-4,5-dihydropyrazolo[1,5-a]pyrazine-2-carboxylic acid (Intermediate 218A, 70.0 mg, 220 μmol), 3-(4-fluorophenyl)-1-methylazetidin-3-amine (Intermediate 46A,43.7 mg, 242 μmol), HATU (101 mg, 264 μmol) and N,N-diisopropylethylamine (120 μl, 660 μmol) in DMF (2.5 ml). The reaction mixture was directly purified by preparative HPLC (Method P14) to afford the title compound. Yield: 53.0 mg (49% of theory).

LC/MS [Method 3]: Rₜ=1.33 min; MS (ESIpos): m/z=480 [M+H]⁺.

¹H-NMR (400 MHz, DMSO-d₆): δ [ppm]=11.55 (br s, 1H), 9.32 (s, 1H), 7.91 (s, 1H), 7.83 (d, 1H), 7.66-7.59 (m, 3H), 7.47 (d, 1H), 7.22-7.14 (m, 2H), 3.94-3.75 (m, 2H), 3.75-3.52 (m, 2H), 2.56 (s, 3H), 2.42 (s, 3H), 2.39 (s, 3H).

### Example 1-151

### 6-(3-Chloro-4-methylphenyl)-N-[(1S)-1-(4-fluorophenyl)-2-hydroxy-2-methylpropyl]-4-oxo-3-(trifluoromethyl)-4,5-dihydropyrazolo[1,5-a]pyrazine-2-carboxamide

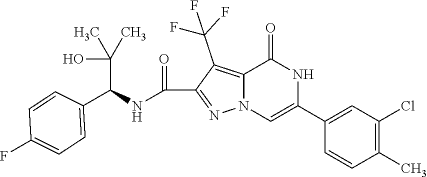 [SMILES: Cc1ccc(-c2cn3nc(C(=O)N[C@@H](c4ccc(F)cc4)C(C)(C)O)c(C(F)(F)F)c3c(=O)[nH]2)cc1Cl]

HATU (246 mg, 646 μmol) and N,N-diisopropylethylamine (280 μl, 1.6 mmol) were added to a solution of 6-(3-chloro-4-methylphenyl)-4-oxo-3-(trifluoromethyl)-4,5-dihydropyrazolo[1,5-a]pyrazine-2-carboxylic acid (Intermediate 221A, 200 mg, 538 μmol) in DMF (20 ml) at RT. After stirring for 5 min, (1S)-1-amino-1-(4-fluorophenyl)-2-methylpropan-2-ol (Intermediate 36A, 108 mg, 592 μmol) was added and stirring was continued for at RT for 2 h. The reaction mixture was directly purified by preparative HPLC (Method P14) to afford the title compound. Yield: 145 mg (50% of theory).

LC/MS [Method 3]: Rₜ=1.97 min; MS (ESIpos): m/z=537 [M+H]⁺.

¹H-NMR (400 MHz, DMSO-d₆): δ [ppm]=12.11 (s, 1H), 8.91 (d, 1H), 8.35 (s, 1H), 7.89 (d, 1H), 7.67 (dd, 1H), 7.50-7.41 (m, 3H), 7.17-7.11 (m, 2H), 4.93 (d, 1H), 4.65 (s, 1H), 2.39 (s, 3H), 1.18 (s, 3H), 1.04 (s, 3H).

### Example 1-152

### N-[(2R)-Butan-2-yl]-6-(3-chloro-4-methylphenyl)-4-oxo-3-(trifluoromethyl)-4,5-dihydropyrazolo-[1,5-a]pyrazine-2-carboxamide

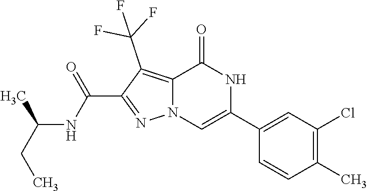 [SMILES: CC[C@@H](C)NC(=O)c1nn2cc(-c3ccc(C)c(Cl)c3)[nH]c(=O)c2c1C(F)(F)F]

The title compound was prepared according to general procedure 1-C starting from 6-(3-chloro-4-methylphenyl)-4-oxo-3-(trifluoromethyl)-4,5-dihydropyrazolo[1,5-a]pyrazine-2-carboxylic acid (Intermediate 221A, 70.0 mg, 188 μmol), (2R)-butan-2-amine (15.2 mg, 207 μmol), HATU (85.9 mg, 226 μmol) and N,N-diisopropylethylamine (98 μl, 560 μmol) in DMF (2.2 ml). The reaction mixture was directly purified by preparative HPLC (Method P14) to afford the title compound. Yield: 10.0 mg (12% of theory).

LC/MS [Method 3]: Rₜ=1.96 min; MS (ESIpos): m/z=427 [M+H]⁺.

¹H-NMR (500 MHz, DMSO-d₆): δ [ppm]=12.11 (br s, 1H), 8.55 (d, 1H), 8.29 (s, 1H), 7.87 (d, 1H), 7.65 (dd, 1H), 7.49 (d, 1H), 3.92-3.84 (m, 1H), 2.39 (s, 3H), 1.52-1.45 (m, 2H), 1.12 (d, 3H), 0.89 (t, 3H).

### Example 1-153

### 6-(3-Chloro-4-methylphenyl)-N-[3-(4-fluorophenyl)-1-methylazetidin-3-yl]-4-oxo-3-(trifluoromethyl)-4,5-dihydropyrazolo[1,5-a]pyrazine-2-carboxamide

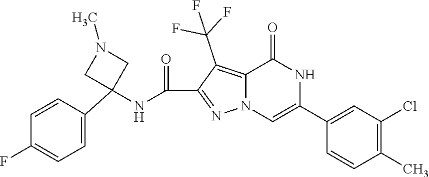 [SMILES: Cc1ccc(-c2cn3nc(C(=O)NC4(c5ccc(F)cc5)CN(C)C4)c(C(F)(F)F)c3c(=O)[nH]2)cc1Cl]

The title compound was prepared according to general procedure 1-C starting from 6-(3-chloro-4-methylphenyl)-4-oxo-3-(trifluoromethyl)-4,5-dihydropyrazolo[1,5-a]pyrazine-2-carboxylic acid (Intermediate 221A, 70.0 mg, 188 μmol), 3-(4-fluorophenyl)-1-methylazetidin-3-amine (Intermediate 46A, 37.3 mg, 207 μmol), HATU (85.9 mg, 226 μmol) and N,N-diisopropylethylamine (98 μl, 560 μmol) in DMF (2.2 ml). The reaction mixture was directly purified by preparative HPLC (Method P14) to afford the title compound. Yield: 20.0 mg (20% of theory).

LC/MS [Method 3]: Rₜ=1.37 min; MS (ESIpos): m/z=534 [M+H]⁺.

¹H-NMR (500 MHz, DMSO-d₆): δ [ppm]=11.81 (br s, 1H), 9.77 (s, 1H), 8.31 (s, 1H), 7.87 (d, 1H), 7.67-7.61 (m, 3H), 7.50 (d, 1H), 7.23-7.18 (m, 2H), 3.79-3.65 (m, 2H), 3.58-3.42 (m, 2H), 2.39 (s, 3H), 2.37 (br s, 3H).

### Example 1-154

### 3-Cyclopropyl-6-(3,4-dimethylphenyl)-N-[(2R)-1-hydroxpropan-2-yl]-4-oxo-4,5-dihydropyrazolo-[1,5-a]pyrazine-2-carboxamide

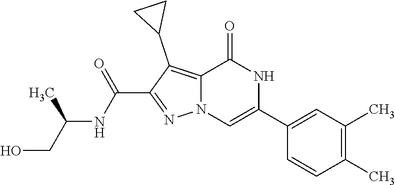 [SMILES: Cc1ccc(-c2cn3nc(C(=O)N[C@H](C)CO)c(C4CC4)c3c(=O)[nH]2)cc1C]

The title compound was prepared according to general procedure 1-C starting from 3-cyclopropyl-6-(3,4-dimethylphenyl)-4-oxo-4,5-dihydropyrazolo[1,5-a]pyrazine-2-carboxylic acid (Intermediate 92A, 70.0 mg, 216 μmol), (2R)-2-aminopropan-1-ol (34 μl, 430 μmol), HATU (103 mg, 271 μmol), N,N-diisopropylethylamine (94 μl, 540 μmol) and DMF (2.8 ml). The reaction mixture was diluted with DMSO (2.0 ml) and directly purified by preparative HPLC (Method P14) to afford the title compound. Yield: 45.2 mg (55% of theory).

LC/MS [Method 3]: Rₜ=1.58 min; MS (ESIpos): m/z=381 [M+H]⁺.

¹H-NMR (400 MHz, DMSO-d₆): δ [ppm]=11.36 (s, 1H), 7.89 (s, 1H), 7.84 (d, 1H), 7.54 (s, 1H), 7.45 (dd, 1H), 7.23 (d, 1H), 4.80 (br s, 1H), 4.05-3.95 (m, 1H), 3.49-3.35 (m, 2H), 2.89-2.81 (m, 1H), 2.27 (s, 3H), 2.26 (s, 3H), 1.28-1.19 (m, 2H), 1.14 (d, 3H), 0.92-0.83 (m, 2H).

### Example 1-155

### 3-Cyclopropyl-6-(3,4-dimethylphenyl)-N-[(2S)-1-hydroxpropan-2-yl]-4-oxo-4,5-dihydropyrazolo-[1,5-a]pyrazine-2-carboxamide

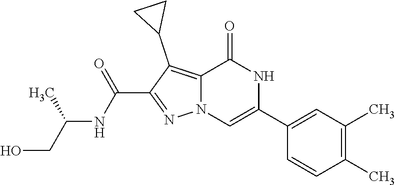 [SMILES: Cc1ccc(-c2cn3nc(C(=O)N[C@@H](C)CO)c(C4CC4)c3c(=O)[nH]2)cc1C]

The title compound was prepared according to general procedure 1-C starting from 3-cyclopropyl-6-(3,4-dimethylphenyl)-4-oxo-4,5-dihydropyrazolo[1 ,5-a]pyrazine-2-carboxylic acid (Intermediate 92A, 70.0 mg, 216 μmol), (2S)-2-aminopropan-1-ol (34 μl, 430 μmol), HATU (103 mg, 271 μmol), N,N-diisopropylethylamine (94 μl, 540 μmol) and DMF (2.8 ml). The reaction mixture was diluted with DMSO (2.0 ml) and directly purified by preparative HPLC (Method P14) to afford the title compound. Yield: 45.5 mg (55% of theory).

LC/MS [Method 3]: Rₜ=1.58 min; MS (ESIpos): m/z=381 [M+H]⁺.

¹H-NMR (400 MHz, DMSO-d₆): δ [ppm]=11.36 (s, 1H), 7.89 (s, 1H), 7.84 (d, 1H), 7.54 (s, 1H), 7.45 (dd, 1H), 7.23 (d, 1H), 4.80 (t, 1H), 4.05-3.95 (m, 1H), 3.49-3.34 (m, 2H), 2.89-2.81 (m, 1H), 2.27 (s, 3H), 2.26 (s, 3H), 1.28-1.19 (m, 2H), 1.14 (d, 3H), 0.91-0.83 (m, 2H).

### Example 1-156

### N-[1-Acetyl-3-(4-methoxyphenyl)azetidin-3-yl]-6-(3,4-dimethylphenyl)-4-oxo-5H-pyrazolo[1,5-a]-pyrazine-2-carboxamide

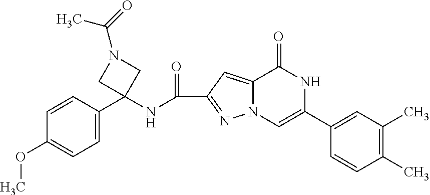 [SMILES: COc1ccc(C2(NC(=O)c3cc4c(=O)[nH]c(-c5ccc(C)c(C)c5)cn4n3)CN(C(C)=O)C2)cc1]

Acetylchloride (13.1 mg, 167 μmol) was added to a mixture of N-[3-(4-fluorophenyl)azetidin-3-yl]-6-(3,4-dimethylphenyl)-4-oxo-4,5-dihydropyrazolo[1,5-a]pyrazine-2-carboxamide hydrochloride (1:1) (Example 1-65, 80 mg, 167 μmol) and N,N-diisopropylethyl amine (87 μl, 500 μmol) in dichloromethane (10 ml) and the mixture was stirred at RT for 4 h. After aqueous work-up, the organic phase was dried over magnesium sulfate and evaporated to provide a crude product. This residue was purified by preparative HPLC (Method P1). After evaporation of product containing fractions one obtained 28.5 mg of the desired compound (yield: 35% of theory; purity: 99% by LCMS).

LC/MS [Method 3]: Rₜ=1.56 min; MS (ESIpos): m/z=486 (M+H)⁺.

¹H-NMR (400 MHz, DMSO-d₆): δ [ppm]=11.7 (br s, 1H), 9.53 (s, 1H), 7.93 (s, 1H), 7.56 (s, 1H), 7.48 (dd, 1H), 7.40 (d, 2H), 7.35 (s, 1H), 7.26 (d, 1H), 6.93 (d, 2H), 4.61-4.50 (m, 2H), 4.36 (d, 1H), 4.08 (d, 1H), 3,74 (s, 3H), 2.29 (s, 3H), 2.28 (s, 3H), 1.86 (s, 3H).

### Example 1-157

### tert-Butyl 3-[(4-fluorophenyl)-[[6-(2-naphthyl)-4-oxo-5H-pyrazolo[1,5-a]pyrazine-2-carbonyl]amino]-methyl]azetidine-1-carboxylate (Racemate)

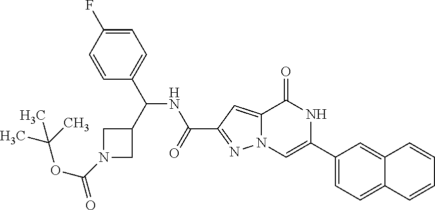 [SMILES: CC(C)(C)OC(=O)N1CC(C(NC(=O)c2cc3c(=O)[nH]c(-c4ccc5ccccc5c4)cn3n2)c2ccc(F)cc2)C1]

6-(2-Naphthyl)-4-oxo-5H-pyrazolo[1,5-a]pyrazine-2-carboxylic acid (Intermediate 3A, 1.00g, 3.28 mmol), HATU (1.37 g, 3.60 mmol) and diisopropylethylamine (1.27g,9.83 mmol) were stirred in DMF (40 ml) at RT for 1 h. Then, racemic tert-butyl 3-[amino(4-fluorophenyl)methyl]azetidine-1-carboxylate (Intermediate 236A, 0.92 g, 3.28 mmol) was added and the mixture was stirred at RT for 72 h. Water was added, the solid was filtered off and washed with water and ethyl acetate. The solid was dried in vacuo, yielding the title compound (1.54 g 77% of theory, 94% purity).

LC/MS [Method 3]: Rₜ=2.11 min; MS (ESIpos): m/z=568 [M+H]⁺.

¹H-NMR (600 MHz, DMSO-d₆): δ [ppm] 11.86 (s, 1H), 9.00 (d, 1H), 8.37 (s, 1H), 8.14 (m, 1H), 8.03 (d, 1H), 7.98 (m, 2H), 7.86 (dd, 1H), 7.59 (m, 2H), 7.53 (dd, 2H), 7.41 (s, 1H), 7.18(t, 2H), 5.29 (t, 1H), 4.0-3.5 (m, 4 H), 3.32 (s, 1H), 3.19 (m, 1H), 1.37 (s, 9H).

### Example 1-158

### 6-(3-fluoro-4-methylphenyl)-N-[(1S)-1-(4-fluorophenyl)-2-hydroxy-2-methylpropyl]-4-oxo-3-(trifluoromethyl)-4,5-dihydropyrazolo[1,5-a]pyrazine-2-carboxamide

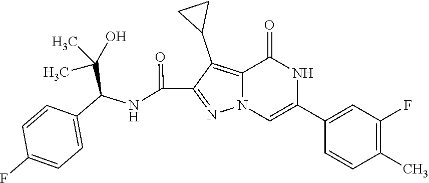 [SMILES: Cc1ccc(-c2cn3nc(C(=O)N[C@@H](c4ccc(F)cc4)C(C)(C)O)c(C4CC4)c3c(=O)[nH]2)cc1F]

A solution of 3-cyclopropyl-6-[3-fluoro-4-methylphenyl]-4-oxo-4,5-dihydropyrazolo[1,5-a]pyrazine-2-carboxylic acid (Intermediate 242A, 50.0 mg, 0.15 mmol), HATU (66 mg, 0.17 mmol) and N,N-diisopropylethylamine (51 μl, 0.29 mmol) in DMF (0.98 ml) was stirred at 0° C. for 10 min before (1S)-1-amino-1-(4-fluorophenyl)-2-methylpropan-2-ol (Intermediate 36A, 30.8 mg, 0.16 mmol) was added. After the addition, stirring was continued at 0° for 30 minutes. Then, water (10 ml) was added, and the precipitate was collected by filtration and washed with water. The crude product was purified by preparative HPLC (gradient of acetonitrile in water). Yield: 40.0 mg (56% of theory).

LC/MS [Method 3]: Rₜ=1.98 min; MS (ESIpos): m/z=493 [M+H]⁺.

¹H-NMR (500 MHz, DMSO-d₆): δ [ppm]=11.45 (br. s, 1H), 8.33 (d, 1H), 8.17 (s, 1H), 7.61 (dd, 1H), 7.55 (dd, 1H), 7.43 (m, 1H), 7.39 (t, 1H), 7.13 (t, 1H), 4.90 (s, 1H), 4.84 (d, 1H), 2.86-2.80 (m, 1H), 2.28 (s, 3H), 1.25(s, 3H), 1.21-1.18 (m, 2H), 0.98 (s, 3H), 0.84-0.82 (m, 2H).

### Example 1-159

### 3-Cyclopropyl-6-(3-fluoro-4-methylphenyl)-4-oxo-N-(3,3,3-trifluoro-2-hydroxpropyl)-4,5-dihydro-pyrazolo[1,5-a]pyrazine-2-carboxamide (racemate)

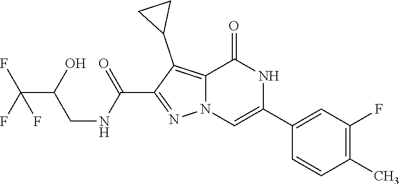 [SMILES: Cc1ccc(-c2cn3nc(C(=O)NCC(O)C(F)(F)F)c(C4CC4)c3c(=O)[nH]2)cc1F]

A solution of 3-cyclopropyl-6-[3-fluoro-4-methylphenyl]-4-oxo-4,5-dihydropyrazolo[1,5-a]pyrazine-2-carboxylic acid (Intermediate 242A, 100 mg, 0.31 mmol), HATU (139 mg, 0.37 mmol) and N,N-diisopropylethylamine (160 μl, 0.92 mmol) in DMF (0.92 ml) was stirred at 0° C. for 5 min before 3-amino-1,1,1-trifluoropropan-2-ol hydrochloride (1:1) (55.6 mg, 0.34 mmol) was added. After the addition, stirring was continued at 0° for 30 minutes. Then, water (25 ml) was added, and the precipitate was collected by filtration, washed with water and dried under vacuum. Yield: 84 mg (61% of theory, 98% purity).

LC/MS [Method 3]: Rₜ=1.75 min; MS (ESIpos): m/z=439 [M+H]⁺.

¹H-NMR (500 MHz, DMSO-d₆): δ [ppm]=11.46 (br. s, 1H), 8.30 (t, 1H), 7.97 (s, 1H), 7.59 (dd, 1H), 7.52 (dd, 1H), 7.39 (t, 1H), 6.50 (d, 1H), 4.25-4.19 (m, 1H), 3.64-3.58 (m, 1H), 3.41-3.35 (m, 1H), 2.90-2.82 (m, 1H), 2.28 (s, 3H), 1.27-1.24 (m, 2H), 0.91-0.86 (m, 2H).

### Example 1-160

### 3-Cyclopropyl-6-(3-fluoro-4-methylphenyl)-4-oxo-N-(3,3,3-trifluoro-2-hydroxpropyl)-4,5-dihydropyrazolo[1,5-a]pyrazine-2-carboxamide (enantiomer1)

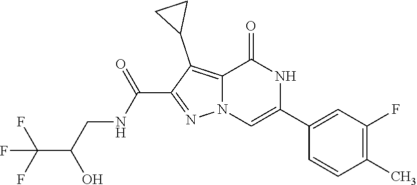 [SMILES: Cc1ccc(-c2cn3nc(C(=O)NCC(O)C(F)(F)F)c(C4CC4)c3c(=O)[nH]2)cc1F]

63.5 mg of the racemic compound from Example 1-159 was separated into its enantiomeres by preparative HPLC (column: Daicel Chiralpak IE, 5 μm, 250×20 mm; flow rate: 15 ml/min; temperature: 60° C.; eluent: 30% n-heptan:70% ethanol). Yield: 20 mg (62% of theory, 99% ee). Chiral analytical HPLC (column: Daicel Chiralpak IE, 5 μm, 250×20 mm; flowrate: 1.0 ml/min; temperature: 45° C.; eluent : 25% i-heptane:75% ethanol): Rₜ=5.75 min.

¹H-NMR (500 MHz, DMSO-d₆): δ [ppm]=11.46 (br. s, 1H), 8.30 (t, 1H), 7.97 (s, 1H), 7.59 (dd, 1H), 7.52 (dd, 1H), 7.39 (t, 1H), 6.50 (d, 1H), 4.25-4.19 (m, 1H), 3.64-3.58 (m, 1H), 3.41-3.35 (m, 1H), 2.90-2.82 (m, 1H), 2.28 (s, 3H), 1.27-1.24 (m, 2H), 0.91-0.86 (m, 2H).

### Example 1-161

### 3-Cyclopropyl-6-(3-fluoro-4-methylphenyl)-4-oxo-N-(3,3,3-trifluoro-2-hydroxpropyl)-4,5-dihydropyrazolo[1,5-a]pyrazine-2-carboxamide (enantiomer2)

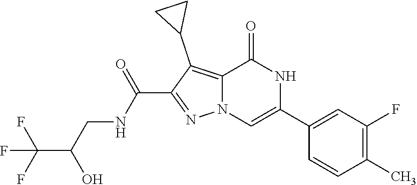 [SMILES: Cc1ccc(-c2cn3nc(C(=O)NCC(O)C(F)(F)F)c(C4CC4)c3c(=O)[nH]2)cc1F]

The compound was obtained as second enantiomer of the preparative HPLC described in Example 1-160. Yield: 21 mg (ee 99%, 62% of theory). chiral analytical HPLC (column: Daicel Chiralpak IE, 5 μm, 250×20 mm; flowrate: 1.0 ml/min; temperature: 45° C.; eluent:25% i-heptane:75% ethanol): Rₜ=7.19 min.

¹H-NMR (500 MHz, DMSO-d₆): δ [ppm]=11.46 (br. s, 1H), 8.30 (t, 1H), 7.97 (s, 1H), 7.59 (dd, 1H), 7.52 (dd, 1H), 7.39 (t, 1H), 6.50 (d, 1H), 4.25-4.19 (m, 1H), 3.64-3.58 (m, 1H), 3.41-3.35 (m, 1H), 2.90-2.82 (m, 1H), 2.28 (s, 3H), 1.27-1.24 (m, 2H), 0.91-0.86 (m, 2H).

### Example 1-162

### 6-(3-Fluoro-4-methylphenyl)-N-[(1S)-1-(4-fluorophenyl)-2-hydroxy-2-methylpropyl]-3-methyl-4-oxo-4,5-dihydropyrazolo[1,5-a]pyrazine-2-carboxamide

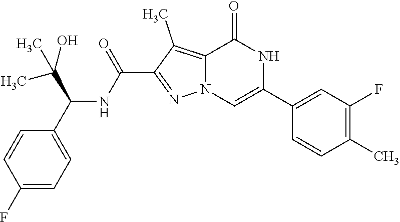 [SMILES: Cc1ccc(-c2cn3nc(C(=O)N[C@@H](c4ccc(F)cc4)C(C)(C)O)c(C)c3c(=O)[nH]2)cc1F]

6-(3-Fluoro-4-methylphenyl)-3-methyl-4-oxo-4,5-dihydropyrazolo[1,5-a]pyrazine-2-carboxylic acid (Intermediate 245A, 60.0 mg, 199 μmol) was suspended in DMF (1.3 ml) and at 0° C., HATU (90.9 mg, 239 μmol) and N,N-diisopropylethylamine (100 μl, 600 μmol) were added. After stirring at 0° C. for 5 min, (1S)-1-amino-1-(4-fluorophenyl)-2-methylpropan-2-ol (Intermediate 36A, 42.3 mg, 95% purity, 219 μmol) was added and stirring was continued at this temperature for 30 min. The reaction mixture was poured into water (10 ml), the precipitate was collected by filtration, washed with water and dried to afford the title compound (72.3 mg, 70% of theory, 90% purity).

LC/MS [Method 7]: Rₜ=1.01 min; MS (ESIneg): m/z=465 [M−H]⁻.

¹H-NMR (500 MHz, DMSO-d₆): δ [ppm]=11.50 (br s, 1H), 8.22 (d, 1H), 8.18 (s, 1H), 7.61 (dd, 1H), 7.54 (dd, 1H), 7.46-7.38 (m, 3H), 7.16-7.08 (m, 2H), 4.99 (s, 1H), 4.82 (d, 1H), 2.63-2.58 (m, 3H), 2.28 (s, 3H), 1.26 (s, 3H), 0.96 (s, 3H).

### Example 1-163

### 6-(2,3-Difluoro-4-methylphenyl)-N-[(1S)-1-(4-fluorophenyl)-2-hydroxy-2-methylpropyl]-3-methyl-4-oxo-4,5-dihydropyrazolo[1,5-a]pyrazine-2-carboxamide

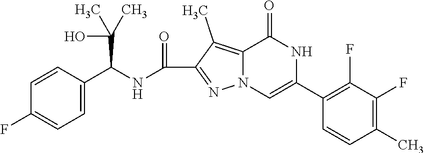 [SMILES: Cc1ccc(-c2cn3nc(C(=O)N[C@@H](c4ccc(F)cc4)C(C)(C)O)c(C)c3c(=O)[nH]2)c(F)c1F]

HATU (71.5 mg, 188 μmol) and N,N-diisopropylethylamine (55 μl, 310 μmol) were added to a suspension of 6-(2,3-difluoro-4-methylphenyl)-3-methyl-4-oxo-4,5-dihydropyrazolo[1,5-a]pyrazine-2-carboxylic acid (Intermediate 249A, 50.0 mg, 157 μmol) in DMF (1.1 ml) at RT. After stirring for 5 min, (1S)-1-amino-1-(4-fluorophenyl)-2-methylpropan-2-ol (Intermediate 36A, 31.6 mg, 172 μmol) was added and stirring was continued at RT for 1 h. The reaction mixture was directly purified by preparative HPLC (Chromatorex C18, 10 pm, 125 mm x 30 mm; gradient of acetonitrile in water containing 0.05% trifluoroactic acid) to afford the title compound (16.0 mg, 21% of theory).

LC/MS [Method 3]: Rₜ=1.91 min; MS (ESIpos): m/z=485 [M+H]⁺.

¹H-NMR (400 MHz, DMSO-d₆): δ [ppm]=11.59 (s, 1H), 8.26 (d, 1H), 7.93 (s, 1H), 7.45-7.38 (m, 2H), 7.38-7.32 (m, 1H), 7.27-7.20 (m, 1H), 7.16-7.09 (m, 2H), 4.98 (s, 1H), 4.84 (d, 1H), 2.59 (s, 3H), 2.39-2.31 (m, 3H), 1.25 (s, 3H), 0.96 (s, 3H).

### Example 1-164

### 6-(2-Fluoro-3,4-dimethylphenyl)-N-[(15)-1-(4-fluorophenyl)-2-hydroxy-2-methylpropyl]-3-methyl-4-oxo-4,5-dihydropyrazolo[1,5-a]pyrazine-2-carboxamide

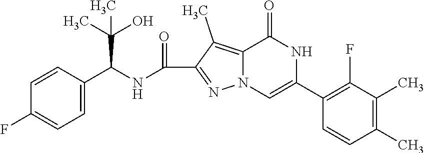 [SMILES: Cc1ccc(-c2cn3nc(C(=O)N[C@@H](c4ccc(F)cc4)C(C)(C)O)c(C)c3c(=O)[nH]2)c(F)c1C]

At 0° C., HATU (72.4 mg, 190 μmol) and N,N-diisopropylethylamine (55 μl, 320 μmol) were added to a suspension of 6-(2-fluoro-3,4-dimethylphenyl)-3-methyl-4-oxo-4,5-dihydropyrazolo[1,5-a]-pyrazine-2-carboxylic acid (Intermediate 254A, 50.0 mg, 159 μmol) in DMF (1.1 ml). After stirring for 5 min, (1S)-1-amino-1-(4-fluorophenyl)-2-methylpropan-2-ol (Intermediate 36A, 33.6 mg, 95% purity, 174 μmol) was added and stirring was continued at 0° C. for 30 min. The reaction mixture was poured into water (10 ml). The precipitate was collected by filtration, washed with water and dried to afford the title compound (40.1 mg, 53% of theory).

LC/MS [Method 3]: Rₜ=1.95 min; MS (ESIpos): m/z=481 [M+H]⁺.

¹H-NMR (400 MHz, DMSO-d₆): δ [ppm]=11.48 (s, 1H), 8.25 (d, 1H), 7.81 (s, 1H), 7.47-7.38 (m, 2H), 7.35-7.28 (m, 1H), 7.16-7.09 (m, 3H), 4.97 (s, 1H), 4.83 (d, 1H), 2.59 (s, 3H), 2.32 (s, 3H), 2.21-2.18 (m, 3H), 1.25 (s, 3H), 0.96 (s, 3H).

### Example 1-165

### 3-Cyclopropyl-6-(3-fluoro-4-methylphenyl)-4-oxo-N-[(2R)-1,1,1-trifluoro-3-hydroxpropan-2-yl]-4,5-dihydropyrazolo[1,5-a]pyrazine-2-carboxamide

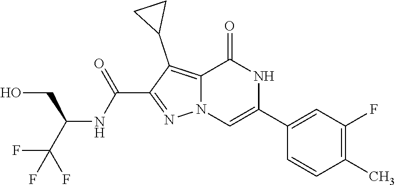 [SMILES: Cc1ccc(-c2cn3nc(C(=O)N[C@H](CO)C(F)(F)F)c(C4CC4)c3c(=O)[nH]2)cc1F]

A mixture of 3-cyclopropyl-6-(3-fluoro-4-methylphenyl)-4-oxo-4,5-dihydropyrazolo[1,5-a]pyrazine-2-carboxylic acid (Intermediate 242A, 120 mg, 367 μmol), HATU (139 mg, 367 μmol) and N,N-diisopropylethylamine (190 μl, 1.1 mmol) in DMF (3.5 ml) was stirred at RT for 10 min. Then, (2R)-2-amino-3,3,3-trifluoropropan-1-ol hydrogen chloride (1/1) (66.8 mg, 403 μmol) was added and stirring was continued at RT overnight. The reaction mixture was diluted with DMSO (2 ml) and directly purified by preparative HPLC (Method P14) to afford the title compound (115 mg, 72% of theory).

LC/MS [Method 3]: Rₜ=1.78 min; MS (ESIpos): m/z=439 [M+H]⁺.

¹H-NMR (400 MHz, DMSO-d₆): δ [ppm]=11.51 (s, 1H), 8.52 (d, 1H), 8.07 (s, 1H), 7.59 (d, 1H), 7.52 (d, 1H), 7.43-7.37 (m, 1H), 5.26 (t, 1H), 4.82-4.71 (m, 1H), 3.82-3.73 (m, 2H), 2.85-2.77 (m, 1H), 2.28 (s, 3H), 1.27-1.20 (m, 2H), 0.95-0.86 (m, 2H).

### Example 1-166

### 6-(4-Chloro-3-methylphenyl)-N-[(1S)-1-(4-fluorophenyl)-2-hydroxy-2-methylpropyl]-4-oxo-3-(trifluoromethyl)-4,5-dihydropyrazolo[1,5-a]pyrazine-2-carboxamide

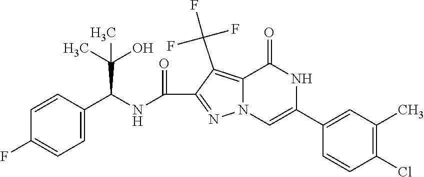 [SMILES: Cc1cc(-c2cn3nc(C(=O)N[C@@H](c4ccc(F)cc4)C(C)(C)O)c(C(F)(F)F)c3c(=O)[nH]2)ccc1Cl]

A mixture of 6-(4-chloro-3-methylphenyl)-4-oxo-3-(trifluoromethyl)-4,5-dihydropyrazolo[1,5-a]pyrazine-2-carboxylic acid (Intermediate 239A, 70.0 mg, 188 μmol), (1S)-1-amino-1-(4-fluorophenyl)-2-methylpropan-2-ol (Intermediate 36A, 38.0 mg, 207 μmol), HATU (71.6 mg, 188 μmol) and N,N-diisopropylethylamine (98 μl, 560 μmol) in DMF (2.2 ml) was stirred at RT for 1 h. The reaction mixture was directly purified by preparative HPLC (Method P14) to afford the title compound (67.6 mg, 67% of theory).

LC/MS [Method 3]: Rₜ=1.05 min; MS (ESIpos): m/z=537 [M+H]⁺.

¹H-NMR (400 MHz, DMSO-d₆): δ [ppm]=12.09 (s, 1H), 8.91 (d, 1H), 8.31 (s, 1H), 7.83 (d, 1H), 7.64 (dd, 1H), 7.55 (d, 1H), 7.47-7.40 (m, 2H), 7.18-7.10 (m, 2H), 4.93 (d, 1H), 4.65 (s, 1H), 2.40 (s, 3H), 1.18 (s, 3H), 1.04 (s, 3H).

### Example 1-167

### 6-(4-Chloro-3-fluorophenyl)-3-cyclopropyl-N-[(15)-1-(4-fluorophenyl)-2-hydroxy-2-methylpropyl]-4-oxo-4,5-dihydropyrazolo[1,5-a]pyrazine-2-carboxamide

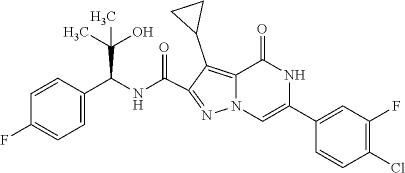 [SMILES: CC(C)(O)[C@@H](NC(=O)c1nn2cc(-c3ccc(Cl)c(F)c3)[nH]c(=O)c2c1C1CC1)c1ccc(F)cc1]

A mixture of 6-(4-chloro-3-fluorophenyl)-3-cyclopropyl-4-oxo-4,5-dihydropyrazolo[1,5-a]pyrazine-2-carboxylic acid (Intermediate 258A, 200 mg, 575 μmol), (1S)-1-amino-1-(4-fluorophenyl)-2-methylpropan-2-ol (Intermediate 36A, 126 mg, 690 μmol), HATU (219 mg, 575 μmol) and N,N-diisopropylethylamine (250 μl, 1.4 mmol) in DMF (8.6 ml) was stirred for 40 min at RT. The reaction mixture was directly purified by preparative HPLC (Method P14) to afford the title compound (149 mg, 51% of theory).

LC/MS [Method 3]: Rₜ=2.03 min; MS (ESIpos): m/z=513 [M+H]⁺.

¹H-NMR (400 MHz, DMSO-d₆): δ [ppm]=11.53 (s, 1H), 8.35 (d, 1H), 8.27 (d, 1H), 7.90 (dd, 1H), 7.74-7.66 (m, 2H), 7.45-7.40 (m, 2H), 7.16-7.10 (m, 2H), 4.90 (s, 1H), 4.84 (d, 1H), 2.86-2.78 (m, 1H), 1.25 (s, 3H), 1.22-1.17 (m, 2H), 0.98 (s, 3H), 0.87-0.81 (m, 2H).

### Example 1-168

### 3-Cyclopropyl-6-(3,4-dichlorophenyl)-N-[(1S)-1-(4-fluorophenyl)-2-hydroxy-2-methylpropyl]-4-oxo-4,5-dihydropyrazolo[1,5-a]pyrazine-2-carboxamide

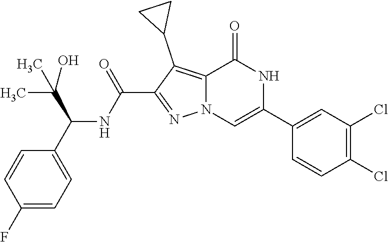 [SMILES: CC(C)(O)[C@@H](NC(=O)c1nn2cc(-c3ccc(Cl)c(Cl)c3)[nH]c(=O)c2c1C1CC1)c1ccc(F)cc1]

HATU (73.1 mg, 192 μmol) and N,N-diisopropylethylamine (67 μl, 480 μmol) were added to a solution of 3-cyclopropyl-6-(3,4-dichlorophenyl)-4-oxo-4,5-dihydropyrazolo[1,5-a]pyrazine-2-carboxylic acid (Intermediate 261A, 70.0 mg, 192 μmol) in DMF (2.5 ml) and the mixture was stirred at RT for 10 min. Then, (1S)-1-amino-1-(4-fluorophenyl)-2-methylpropan-2-ol (Intermediate 36A, 50.3 mg, 70% purity, 192 μmol) was added and stirring was continued overnight. The reaction mixture was diluted with DMSO (2.0 ml) and the resulting solution was directly purified by preparative HPLC (Method P14) to afford the title compound (45.8 mg, 45% of theory).

LC/MS [Method 3]: Rₜ=2.12 min; MS (ESIpos): m/z=529 [M+H]⁺.

¹H-NMR (400 MHz, DMSO-d₆): δ [ppm]=11.54 (s, 1H), 8.34 (d, 1H), 8.26 (d, 1H), 8.10 (d, 1H), 7.80-7.73 (m, 2H), 7.46-7.40 (m, 2H), 7.17-7.10 (m, 2H), 4.90 (s, 1H), 4.84 (d, 1H), 2.86-2.78 (m, 1H), 1.25 (s, 3H), 1.22-1.17 (m, 2H), 0.98 (s, 3H), 0.88-0.79 (m, 2H).

### Example 1-169

### 6-(4-Chloro-3-fluorophenyl)-3-cyclopropyl-N-[3-(4-fluorophenyl)-1-methylazetidin-3-yl]-4-oxo-4,5-dihydropyrazolo[1,5-a]pyrazine-2-carboxamide

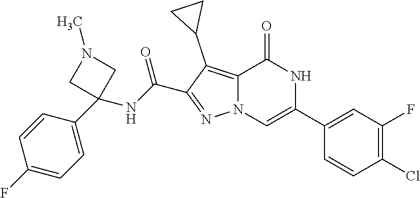 [SMILES: CN1CC(NC(=O)c2nn3cc(-c4ccc(Cl)c(F)c4)[nH]c(=O)c3c2C2CC2)(c2ccc(F)cc2)C1]

A mixture of 6-(4-chloro-3-fluorophenyl)-3-cyclopropyl-4-oxo-4,5-dihydropyrazolo[1,5-a]pyrazine-2-carboxylic acid (Intermediate 258A, 70.0 mg, 201 μmol), 3-(4-fluorophenyl)-1-methylazetidin-3-amine (Intermediate 46A, 39.9 mg, 221 μmol), HATU (76.5 mg, 201 μmol) and N,N-diisopropylethylamine (110 μl, 600 μmol) in DMF (3.0 ml) was stirred at RT overnight. The reaction mixture was directly purified by preparative HPLC (Method P14) to afford the title compound (86.0 mg, 81% of theory, 97% purity).

LC/MS [Method 3]: Rₜ=1.32 min; MS (ESIpos): m/z=510 [M+H]⁺.

¹H-NMR (500 MHz, DMSO-d₆): δ [ppm]=11.54 (br s, 1H), 9.36 (s, 1H), 8.08 (s, 1H), 7.86 (dd, 1H), 7.74-7.69 (m, 1H), 7.66-7.61 (m, 3H), 7.21-7.16 (m, 2H), 3.83-3.64 (m, 2H), 3.61-3.45 (m, 2H), 2.74-2.67 (m, 1H), 2.37 (s, 3H), 1.14-1.08 (m, 2H), 0.87-0.81 (m, 2H).

### Example 1-170

### 3-Cyclopropyl-6-(3,4-dichlorophenyl)-N-[3-(4-fluorophenyl)-1-methylazetidin-3-yl]-4-oxo-4,5-dihydropyrazolo[1,5-a]pyrazine-2-carboxamide

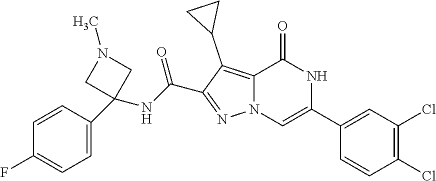 [SMILES: CN1CC(NC(=O)c2nn3cc(-c4ccc(Cl)c(Cl)c4)[nH]c(=O)c3c2C2CC2)(c2ccc(F)cc2)C1]

HATU (73.1 mg, 192 μmol) and N,N-diisopropylethylamine (100 μl, 580 μmol) were added to a solution of 3-cyclopropyl-6-(3,4-dichlorophenyl)-4-oxo-4,5-dihydropyrazolo[1,5-a]pyrazine-2-carboxylic acid (Intermediate 261A, 70.0 mg, 192 μmol) in DMF (1.8 ml) and the mixture was stirred at RT for 10 min. Then, 3-(4-fluorophenyl)-1-methylazetidin-3-amine (Intermediate 46A, 38.1 mg, 211 μmol) was added and stirring was continued overnight. The reaction mixture was diluted with DMSO (2.0 ml) and the resulting solution was directly purified by preparative HPLC (Method P14) to afford the title compound (57.2 mg, 57% of theory).

LC/MS [Method 3]: Rₜ=1.62 min; MS (ESIpos): m/z=526 [M+H]⁺.

¹H-NMR (500 MHz, DMSO-d₆): δ [ppm]=11.55 (br s, 1H), 9.36 (s, 1H), 8.08 (s, 1H), 8.06 (d, 1H), 7.77-7.73 (m, 2H), 7.65-7.60 (m, 2H), 7.21-7.16 (m, 2H), 3.84-3.70 (m, 2H), 3.60-3.47 (m, 2H), 2.74-2.68 (m, 1H), 2.38 (s, 3H), 1.14-1.09 (m, 2H), 0.86-0.82 (m, 2H).

### Example 1-171

### 6-(4-Chloro-3-methylphenyl)-N-[(1S)-2,2-difluoro-1-(4-fluorophenyl)-3-hydroxpropyl]-4-oxo-3-(trifluoromethyl)-4,5-dihydropyrazolo[1,5-a]pyrazine-2-carboxamide

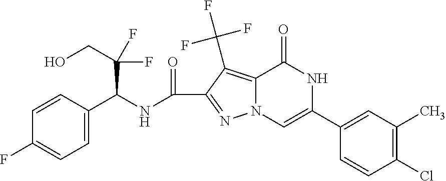 [SMILES: Cc1cc(-c2cn3nc(C(=O)N[C@@H](c4ccc(F)cc4)C(F)(F)CO)c(C(F)(F)F)c3c(=O)[nH]2)ccc1Cl]

HATU (511.5 mg, 1.3 mmol) and N,N-diisopropylethylamine (469 μl, 2.7 mmol) were added to a solution of 6-(4-chloro-3-methylphenyl)-4-oxo-3-(trifluoromethyl)-4,5-dihydropyrazolo[1,5-a]pyrazine-2-carboxylic acid (Intermediate 239A, 500.0 mg, 1.3 mmol) in DMF (6.5 ml) and the mixture was stirred at 0° C. for 10 min. Then, (3S)-3-amino-2,2-difluoro-3-(4-fluorophenyl)propan-1-ol (Intermediate 265A, 289.8 mg, 1.4 mmol) was added and the mixture was stirred at 0° C. for 30 min.

The reaction mixture was quenched with 50 mL of water, filtered, washed with a mixture of MeCN/H₂O 1:1 and lyophilized. The resulting solid was purified by silica gel column chromatography (50g SNAP-Ultra column DCM/7M NH₃ in methanol 20:1) to afford the title compound (439.8 mg, 59% of theory).

LC/MS [Method 3]: Rₜ=1.93 min; MS (ESIpos): m/z=557 [M−H]⁻.

¹H-NMR (500 MHz, DMSO-d₆): δ [ppm]=12.15 (s, 1H), 9.80 (d, 1H), 8.31 (s, 1H), 7.82 (s, 1H), 7.63 (dd, 1H), 7.53-7.59 (m, 3H), 7.26 (t, 2H), 5.68-5.77 (m, 2H), 3.65 (m, 1H), 3.35-3.58 (m, 1H), 2.40 (s, 3H).

### Example 1-172

### 6-(4-Chloro-3-methylphenyl)-3-cyclopropyl-N-[(1S)-2,2-difluoro-1-(4-fluorophenyl)-3-hydroxpropyl]-4-oxo-4,5-dihydropyrazolo[1,5-a]pyrazine-2-carboxamide

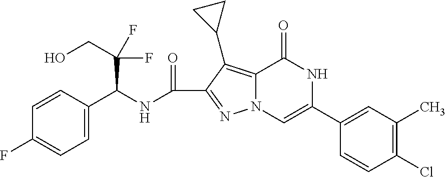 [SMILES: Cc1cc(-c2cn3nc(C(=O)N[C@@H](c4ccc(F)cc4)C(F)(F)CO)c(C4CC4)c3c(=O)[nH]2)ccc1Cl]

HATU (66.4 mg, 175 μmol) and N,N-diisopropylethylamine (91 μl, 524 μmol) were added to a solution of 6-(4-chloro-3-methylphenyl)-3-cyclopropyl-4-oxo-4,5-dihydropyrazolo[1,5-a]pyrazine-2-carboxylic acid (Intermediate 268A, 60 mg, 175 μmol) in DMF (950 μl) and the mixture was stirred at 0° C. for 10 min. Then, (3S)-3-amino-2,2-difluoro-3-(4-fluorophenyl)propan-1-ol (Intermediate 265A, 37.6 mg, 183 μmol) was added and the mixture was stirred at 0° C. for 45 min. The reaction mixture was quenched with formic acid and directly purified by preparative HPLC (Method P16) to afford the title compound (55 mg, 59% of theory).

LC/MS [Method 7]: Rₜ=1.13 min; MS (ESIpos): m/z=531 [M+H]⁺.

¹H-NMR (500 MHz, DMSO-d₆): δ [ppm]=11.50 (s, 1H), 9.05 (d, 1H), 8.09 (s, 1H), 7.80 (d, 1H), 7.56-7.63 (m, 3H), 7.52 (d, 1H), 7.25 (t, 2H), 5.79 (t, 1H), 5.70 (m, 1H), 3.59-3.70 (m, 1H), 3.55 (m, 1H), 2.69 (m, 1H), 2.39 (s, 3H), 1.07-1.15 (m, 2H), 0.84 (dd, 2H).

### Example 1-173

### 6-(4-Chloro-3-methylphenyl)-3-cyclopropyl-N-[(1S)-1-(4-fluorophenyl)-2-hydroxy-2-methylpropyl]-4-oxo-4,5-dihydropyrazolo[1,5-a]pyrazine-2-carboxamide

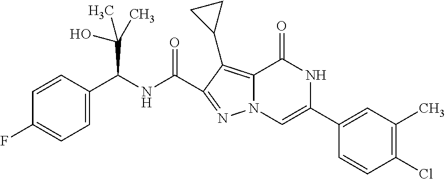 [SMILES: Cc1cc(-c2cn3nc(C(=O)N[C@@H](c4ccc(F)cc4)C(C)(C)O)c(C4CC4)c3c(=O)[nH]2)ccc1Cl]

HATU (79.6 mg, 209 μmol) and N,N-diisopropylethylamine (91 μl, 524 μmol) were added to a solution of 6-(4-Chloro-3-methylphenyl)-3-cyclopropyl-4-oxo-4,5-dihydropyrazolo[1,5-a]pyrazine-2-carboxylic acid (Intermediate 268A, 60 mg, 175 μmol) in DMF (950 pl) and the mixture was stirred at 0° C. for 10 min. Then, (1S)-1-amino-1-(4-fluorophenyl)-2-methylpropan-2-ol (Intermediate 36A, 35.2 mg, 192 μmol) was added and the mixture was stirred at 0° C. for 30 min. The reaction mixture was quenched with 10 mL of water, filtered, washed with a mixture of water and concentrated. The resulting solid was suspended in 3 ml of acetonitrile and stirred overnight. The resulting solid was filtered and dried, to give the title compound (66 mg, 74% of theory).

LC/MS [Method 3]: Rₜ=2.11 min; MS (ESIpos): m/z=509 [M+H]⁺.

¹H-NMR (500 MHz, DMSO-d₆): δ [ppm]=11.45 (s, 1H), 8.33 (d, 1H), 8.13 (s, 1H), 7.82 (s, 1H), 7.63 (d, 1H), 7.51 (d, 1H), 7.43 (t, 2H), 7.13 (t, 2H), 4.90 (s, 1H), 4.84 (d, 1H), 2.78-2.88 (m, 1H), 2.38 (s, 3H), 1.25 (br s, 3H), 1.20 (br s, 2H), 0.98 (br s, 3H), 0.83 (br d, 2H).

### Example 1-174

### tert-Butyl 3-{[6-(3,4-dimethylphenyl)-4-oxo-3-(trifluoromethyl)-4,5-dihydropyrazolo[1,5-a]pyrazine-2-carbonyl]amino}-3-(4-fluorophenyl)azetidine-1-carboxylate

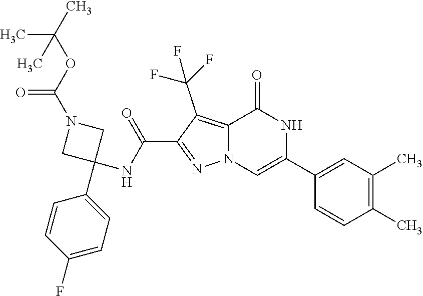 [SMILES: Cc1ccc(-c2cn3nc(C(=O)NC4(c5ccc(F)cc5)CN(C(=O)OC(C)(C)C)C4)c(C(F)(F)F)c3c(=O)[nH]2)cc1C]

The title compound was prepared according to general procedure 1-C starting from 6-(3,4-dimethylphenyl)-4-oxo-3-(trifluoromethyl)-4,5-dihydropyrazolo[1,5-a]pyrazine-2-carboxylic acid (Intermediate 125A, 50 mg, 427 μmol), tert-butyl 3-amino-3-(4-fluorophenyl)azetidine-1-carboxylate (Intermediate 103A,114 mg, 427 μmol), HATU (203 mg, 534 μmol) and N,N-diisopropylethylamine (190 μl, 1.1 mmol) in DMF (6.9 ml). The reaction mixture was directly purified by preparative HPLC (Method P14) to afford the title compound. Yield: 82.0 mg (32% of theory).

LC/MS [Method 4]: Rₜ=1.87 min; MS (ESIneg): m/z=598 [M−H]⁻.

¹H-NMR (400 MHz, DMSO-d₆): δ [ppm]=12.07 (br s, 1H), 9.94 (s, 1H), 8.18 (s, 1H), 7.59 (s, 1H), 7.54-7.48 (m, 3H), 7.29-7.22 (m, 3H), 4.33-4.23 (m, 2H), 4.23-4.12 (m, 2H), 2.29 (s, 3H), 2.28 (s, 3H), 1.42 (s, 9H).

### Example 1-175

### 6-(3,4-Dimethylphenyl)-N-[3-(4-fluorophenyl)azetidin-3-yl]-4-oxo-3-(trifluoromethyl)-4,5-dihydropyrazolo[1,5-a]pyrazine-2-carboxamide-hydrogen chloride (1/1)

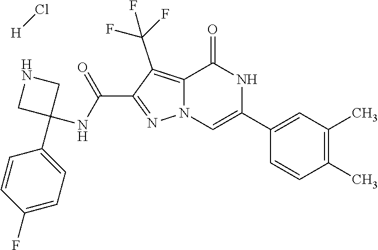

A mixture of tert-butyl 3-{[6-(3,4-dimethylphenyl)-4-oxo-3-(trifluoromethyl)-4,5-dihydropyrazolo-[1,5-a]pyrazine-2-carbonyl]amino}-3-(4-fluorophenyl)azetidine-1-carboxylate (Example 1-174, 82.0 mg, 137 μmol) and hydrogen chloride (4.0 M solution in dioxane, 3.0 ml, 1.4 mmol) in dichloromethane (5.0 ml) was stirred at RT overnight. The mixture was concentrated to dryness and the residue was taken up in water and acetonitrile and lyophilized to afford the title compound. Yield: 76.0 mg (93% of theory, 90% purity).

LC/MS [Method 3]: Rₜ=1.33 min; MS (ESIpos): m/z=500 [M+H]⁺.

### Example 1-176

### 6-(3,4-Dimethylphenyl)-N-[1-ethyl-3-(4-fluorophenyl)azetidin-3-yl]-4-oxo-3-(trifluoromethyl)-4,5-dihydropyrazolo[1,5-a]pyrazine-2-carboxamide

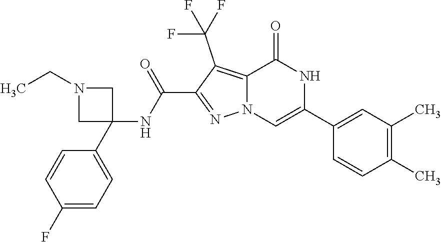 [SMILES: CCN1CC(NC(=O)c2nn3cc(-c4ccc(C)c(C)c4)[nH]c(=O)c3c2C(F)(F)F)(c2ccc(F)cc2)C1]

A mixture of 6-(3,4-dimethylphenyl)-N-[3-(4-fluorophenyl)azetidin-3-yl]-4-oxo-3-(trifluoromethyl)-4,5-dihydropyrazolo[1,5-a]pyrazine-2-carboxamide-hydrogen chloride (1/1) (Example 1-175, 76.0 mg, 90% purity, 128 μmol) and acetaldehyde (1.4 ml, 26 mmol) in acetic acid (1.4 ml) was stirred at RT for 30 min before sodium cyanoborohydride (24.1 mg, 383 μmol) was added. The mixture was stirred at RT overnight. Then, methanol (1.0 ml) and 1.0 M hydrochloric acid (10 drops) were added after stirring for 5 min, the mixture was directly purified by preparative HPLC (Method P14) to afford the title compound. Yield: 56.0 mg (82% of theory, 98% purity).

LC-MS [Method 3]: Rₜ=1.38 min; MS (ESIpos): m/z=528 [M+H]⁺.

¹H-NMR (400 MHz, DMSO-d₆): δ [ppm]=12.03 (br s, 1H), 9.74 (s, 1H), 8.19 (s, 1H), 7.67-7.61 (m, 2H), 7.61-7.57 (m, 1H), 7.50 (dd, 1H), 7.27 (d, 1H), 7.24-7.18 (m, 2H), 3.81-3.54 (m, 2H), 3.53-3.35 (m, 2H), 2.29 (s, 3H), 2.28 (s, 3H), 0.93 (t, 3H).

### Example 1-177

### N-[1-(1,3-Difluoropropan-2-yl)-3-(4-fluorophenyl)azetidin-3-yl]-6-(naphthalen-2-yl)-4-oxo-3-(trifluoro-methyl)-4,5-dihydropyrazolo[1,5-a]pyrazine-2-carboxamide

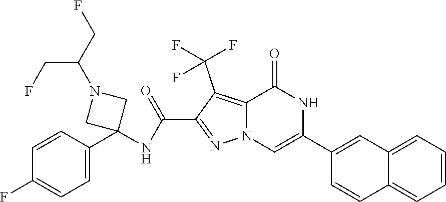 [SMILES: O=C(NC1(c2ccc(F)cc2)CN(C(CF)CF)C1)c1nn2cc(-c3ccc4ccccc4c3)[nH]c(=O)c2c1C(F)(F)F]

A mixture of N-[3-(4-fluorophenyl)azetidin-3-yl]-6-(naphthalen-2-yl)-4-oxo-3-(trifluoromethyl)-4,5-dihydropyrazolo[1,5-a]pyrazine-2-carboxamide (Example 1-125, 27.0 mg, 51.8 μmol) and 1,3-difluoropropan-2-one (48.7 mg, 518 μmol) in acetic acid was stirred at RT for 20 min before sodium cyanoborohydride (16.3 mg, 259 μmol) was added. The mixture was stirred at RT overnight. More 1,3-difluoropropan-2-one (48.7 mg, 518 μmol) and sodium cyanoborohydride (16.3 mg, 259 μmol) were added and stirring was continued in a sealed vessel overnight. The mixture was directly purified by preparative HPLC (Method P14) to afford the title compound. Yield: 8.0 mg (26% of theory).

LC-MS [Method 3]: Rₜ=1.87 min; MS (ESIpos): m/z=600 [M+H]⁺.

¹H-NMR (400 MHz, DMSO-d₆): δ [ppm]=9.75 (s, 1H), 8.45 (s, 1H), 8.40-8.31 (m, 2H), 8.04-7.93 (m, 4H), 7.72-7.62 (m, 2H), 7.62-7.55 (m, 2H), 7.27-7.19 (m, 2H), 4.59-4.49 (m, 2H), 4.48-4.36 (m, 2H), 3.79 (d, 2H), 3.70 (d, 2H).

General Procedure 2 (Parallel Synthesis of Example Compounds):

6-(2-Naphthyl)-4-oxo-4,5-dihydropyrazolo[1,5-a]pyrazine-2-carboxylic acid (Intermediate 3A, 30.5 mg, 100 μmol) and HATU (49.4 mg, 130 μmol) were dissolved in N,N-dimethylformamide (800 μl) and added to the corresponding amine (100 μmol) which was placed on a multi-well plate. N-Methylmorpholine (50 μl, 393 μmol) was added, and the plate was shaken at RT overnight. After this, the reaction mixture was filtered, and the filtrate solution was directly purified by preparative HPLC (Method P11). The fractions containing the desired product were combined, evaporated and dried in vacuo.

The example compounds specified in the following Table 1 were prepared according to general procedure 2:

TABLE 1

| | IUPAC name/structure | |
|---|---|---|
| Example | (yield, purity) | Analytical data |
| 2-1 | 6-(2-Naphthyl)-4-oxo-N-(1-phenylethyl)-5H-pyrazolo- | LC/MS [Method 31]: |
| | [1,5-a]pyrazine-2-carboxamide 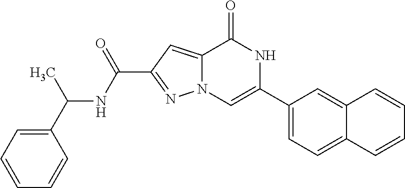 [SMILES: CC(NC(=O)c1cc2c(=O)[nH]c(-c3ccc4ccccc4c3)cn2n1)c1ccccc1] | Rₜ = 1.14 min; MS (ESIpos): m/z = 409 [M + H]⁺. |
| | (59% of theory, 96% purity) | |
| 2-2 | N-[3-Hydroxy-1-(4-methoxyphenyl)propyl]-6- | LC/MS [Method 31]: |
| | (2-naphthyl)-4-oxo-4,5-dihydropyrazolo[1,5-a]pyrazine- | Rₜ = 1.03 min; MS |
| | 2-carboxamide 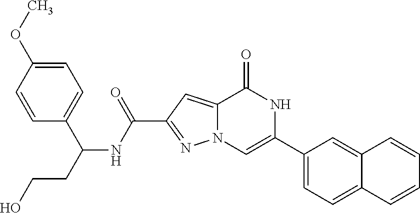 [SMILES: COc1ccc(C(CCO)NC(=O)c2cc3c(=O)[nH]c(-c4ccc5ccccc5c4)cn3n2)cc1] | (ESIpos): m/z = 469 [M + H]⁺. |
| | (43% of theory, 95% purity) | |
| 2-3 | N-[1-(4-Methoxyphenyl)ethyl]-6-(2-naphthyl)-4-oxo- | LC/MS [Method 31]: |
| | 4,5-dihydropyrazolo[1,5-a]pyrazine-2-carboxamide 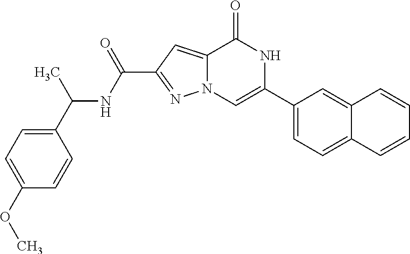 [SMILES: COc1ccc(C(C)NC(=O)c2cc3c(=O)[nH]c(-c4ccc5ccccc5c4)cn3n2)cc1] | Rₜ = 1.14 min; MS (ESIpos): m/z = 439 [M + H]⁺. |
| | (6% yield, 100% purity) | |
| 2-4 | N-(2,2-Difluoro-1-phenylethyl)-6-(2-naphthyl)-4-oxo- | LC/MS [Method 31]: |
| | 4,5-dihydropyrazolo[1,5-a]pyrazine-2-carboxamide 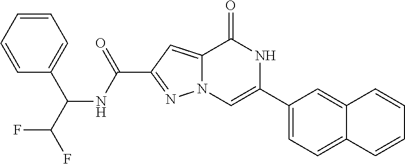 [SMILES: O=C(NC(c1ccccc1)C(F)F)c1cc2c(=O)[nH]c(-c3ccc4ccccc4c3)cn2n1] | Rₜ = 1.16 min; MS (ESIpos): m/z = 445 [M + H]⁺. |
| | (35% yield, 100% purity) | |
| 2-5 | N-[1-(1,3-Dimethyl-1H-pyrazol-4-yl)ethyl]-6-(2- | LC/MS [Method 31]: |
| | naphthyl)-4-oxo-4,5-dihydropyrazolo[1,5-a]pyrazine-2- | Rₜ = 1.05 min; MS |
| | carboxamide 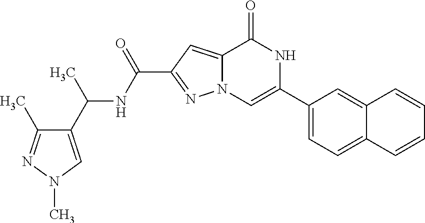 [SMILES: Cc1nn(C)cc1C(C)NC(=O)c1cc2c(=O)[nH]c(-c3ccc4ccccc4c3)cn2n1] | (ESIpos): m/z = 427 [M + H]⁺. |
| | (83% yield, 100% purity) | |
| 2-6 | 6-(2-Naphthy)-N-[(1S)-1-(1-naphthyl)ethyl-4-oxo-4,5- | LC/MS [Method 31]: |
| | dihydropyrazolo[1,5-a]pyrazine-2-carboxamide 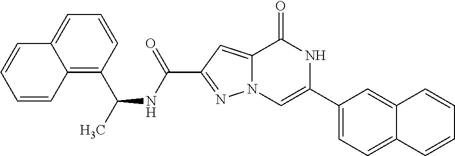 [SMILES: C[C@H](NC(=O)c1cc2c(=O)[nH]c(-c3ccc4ccccc4c3)cn2n1)c1cccc2ccccc12] | Rₜ = 1.21 min; MS (ESIpos): m/z = 459 [M + H]⁺. |
| | (27% yield, 100% purity) | |
| 2-7 | N-[2-(4-Fluorophenyl)ethyl]-6-(2-naphthyl)-4-oxo-4,5- | LC/MS [Method 31]: |
| | dihydropyrazolo[1,5-a]pyrazine-2-carboxamide 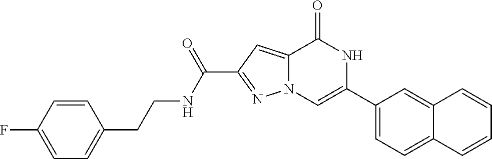 [SMILES: O=C(NCCc1ccc(F)cc1)c1cc2c(=O)[nH]c(-c3ccc4ccccc4c3)cn2n1] | Rₜ = 1.16 min; MS (ESIpos): m/z = 427 [M + H]⁺. |
| | (5% yield, 100% purity) | |
| 2-8 | N-[1-(2-Methoxyphenyl)ethyl]-6-(2-naphthyl)-4-oxo- | LC/MS [Method 31]: |
| | 4,5-dihydropyrazolo[1,5-a]pyrazine-2-carboxamide 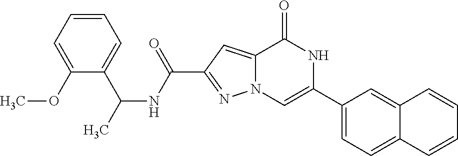 [SMILES: COc1ccccc1C(C)NC(=O)c1cc2c(=O)[nH]c(-c3ccc4ccccc4c3)cn2n1] | Rₜ = 1.18 min; MS (ESIpos): m/z = 439 [M + H]⁺. |
| | (67% yield, 95% purity) | |
| 2-9 | N-[2-(2-Fluorophenyl)ethyl]-6-(2-naphthyl)-4-oxo-4,5- | LC/MS [Method 31]: |
| | dihydropyrazolo[1,5-a]pyrazine-2-carboxamide 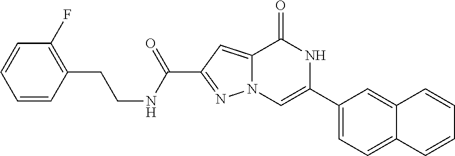 [SMILES: O=C(NCCc1ccccc1F)c1cc2c(=O)[nH]c(-c3ccc4ccccc4c3)cn2n1] | Rₜ = 1.16 min; MS (ESIpos): m/z = 427 [M + H]⁺. |
| | (44% yield, 90% purity) | |
| 2-10 | N-[2-(4-Chlor-1H-pyrazol-1-yl)butyl]-6-(2-naphthyl)-4- | LC/MS [Method 31]: |
| | oxo-4,5-dihydropyrazolo[1,5-a]pyrazine-2-carboxamide 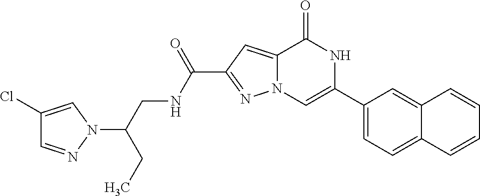 [SMILES: CCC(CNC(=O)c1cc2c(=O)[nH]c(-c3ccc4ccccc4c3)cn2n1)n1cc(Cl)cn1] | Rₜ = 1.12 min; MS (ESIpos): m/z = 461 [M + H]⁺. |
| | (54% yield, 100% purity) | |
| 2-11 | N-(2,2-Dimethylpropyl)-6-(2-naphthyl)-4-oxo-4,5- | LC/MS [Method 31]: |
| | dihydropyrazolo[1,5-a]pyrazine-2-carboxamide 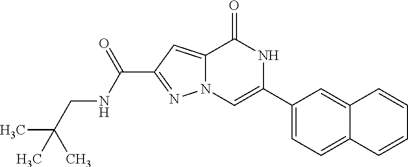 [SMILES: CC(C)(C)CNC(=O)c1cc2c(=O)[nH]c(-c3ccc4ccccc4c3)cn2n1] | Rₜ = 1.14 min; MS (ESIpos): m/z = 375 [M + H]⁺. |
| | (43% yield, 96% purity) | |
| 2-12 | N-[3-Methyl-2-(4-methyl-1H-pyrazol-1-yl)butyl]-6- | LC/MS [Method 31]: |
| | (2-naphthyl)-4-oxo-4,5-dihydropyrazolo[1,5-a]pyrazine- | Rₜ = 1.13 min; MS |
| | 2-carboxamide 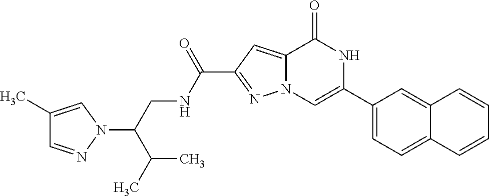 [SMILES: Cc1cnn(C(CNC(=O)c2cc3c(=O)[nH]c(-c4ccc5ccccc5c4)cn3n2)C(C)C)c1] | (ESIpos): m/z = 455 [M + H]⁺. |
| | (96% yield, 98% purity) | |
| 2-13 | N-[2,2-Difluoro-1-(3-fluorophenyl)ethyl]-6-(2-naphthyl)- | LC/MS [Method 31]: |
| | 4-oxo-4,5-dihydropyrazolo[1,5-a]pyrazine-2-carbox- | Rₜ = 1.17 min; MS |
| | amide 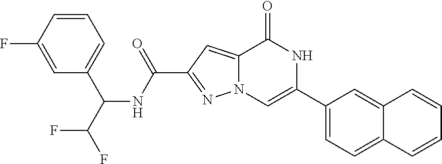 [SMILES: O=C(NC(c1cccc(F)c1)C(F)F)c1cc2c(=O)[nH]c(-c3ccc4ccccc4c3)cn2n1] | (ESIpos): m/z = 463 [M + H]⁺. |
| | (18% yield, 98% purity) | |
| 2-14 | N-(4-Methylbenzyl)-6-(2-naphthyl)-4-oxo-4,5-dihydro- | LC/MS [Method 31]: |
| | pyrazolo[1,5-a]pyrazine-2-carboxamide 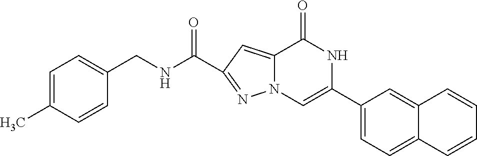 [SMILES: Cc1ccc(CNC(=O)c2cc3c(=O)[nH]c(-c4ccc5ccccc5c4)cn3n2)cc1] | Rₜ = 1.15 min; MS (ESIpos): m/z = 409 [M + H]⁺. |
| | (3% yield, 91% purity) | |
| 2-15 | N-{2-(Dimethylamino)-2-[3-(trifluormethyl)phenyl]ethyl}- | LC/MS [Method 31]: |
| | 6-(2-naphthyl)-4-oxo-4,5-dihydropyrazolo[1,5-a]- | Rₜ = 0.81 min; MS |
| | pyrazine-2-carboxamide 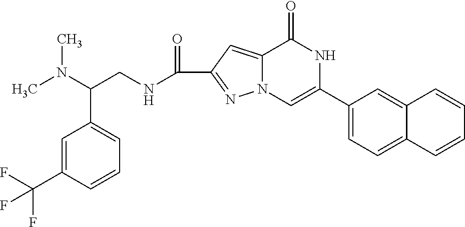 [SMILES: CN(C)C(CNC(=O)c1cc2c(=O)[nH]c(-c3ccc4ccccc4c3)cn2n1)c1cccc(C(F)(F)F)c1] | (ESIpos): m/z = 520 [M + H]⁺. |
| | (82% yield, 100% purity) | |
| 2-16 | N-[6-Methoxy-2,2-dimethyl-3,4-dihydro-2H-chromen- | LC/MS [Method 31]: |
| | 4-yl]-6-(2-naphthyl)-4-oxo-4,5-dihydropyrazolo[1,5-a]- | Rₜ = 1.18 min; MS |
| | pyrazine-2-carboxamide 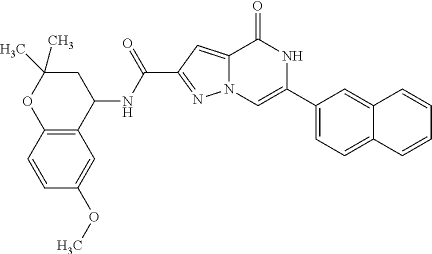 [SMILES: COc1ccc2c(c1)C(NC(=O)c1cc3c(=O)[nH]c(-c4ccc5ccccc5c4)cn3n1)CC(C)(C)O2] | (ESIpos): m/z = 495 [M + H]⁺. |
| | (2% yield, 100% purity) | |
| 2-17 | N-[3-Methyl-2-(1H-pyrazol-1-yl)butyl]-6-(2-naphthyl)- | LC/MS [Method 31]: |
| | 4-oxo-4,5-dihydropyrazolo[1,5-a]pyrazine-2-carbox- | Rₜ = 1.10 min; MS |
| | amide 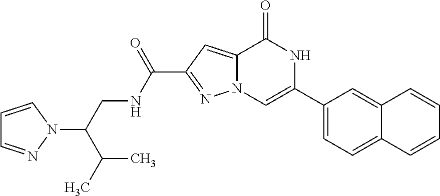 [SMILES: CC(C)C(CNC(=O)c1cc2c(=O)[nH]c(-c3ccc4ccccc4c3)cn2n1)n1cccn1] | (ESIpos): m/z = 441 [M + H]⁺. |
| | (47% yield, 98% purity) | |
| 2-18 | N-[2-(4-Chlor-3-methyl-1H-pyrazol-1-yl)propyl]-6- | LC/MS [Method 31]: |
| | (2-naphthyl)-4-oxo-4,5-dihydropyrazolo[1,5-a]pyrazine- | Rₜ = 1.11 min; MS |
| | 2-carboxamide 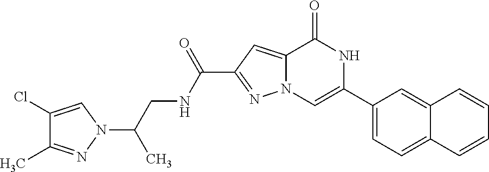 [SMILES: Cc1nn(C(C)CNC(=O)c2cc3c(=O)[nH]c(-c4ccc5ccccc5c4)cn3n2)cc1Cl] | (ESIpos): m/z = 461 [M + H]⁺. |
| | (70% yield, 100% purity) | |
| 2-19 | N-[2-(3,5-Dimethyl-1H-pyrazol-1-yl)-3-methylbutyl]-6- | LC/MS [Method 31]: |
| | (2-naphthyl)-4-oxo-4,5-dihydropyrazolo[1,5-a]pyrazine- | Rₜ = 1.13 min; MS |
| | 2-carboxamide 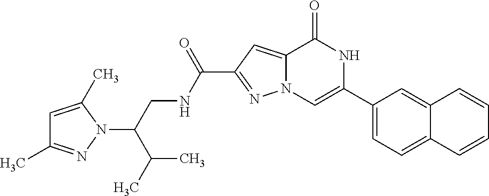 [SMILES: Cc1cc(C)n(C(CNC(=O)c2cc3c(=O)[nH]c(-c4ccc5ccccc5c4)cn3n2)C(C)C)n1] | (ESIpos): m/z = 469 [M + H]⁺. |
| | (60% yield, 99% purity) | |
| 2-20 | N-[2-(4-Chloro-3,5-dimethyl-1H-pyrazol-1-yl)-3-methyl- | LC/MS [Method 31]: |
| | butyl]-6-(2-naphthyl)-4-oxo-4,5-dihydropyrazolo[1,5-a]- | Rₜ = 1.21 min; MS |
| | pyrazine-2-carboxamide 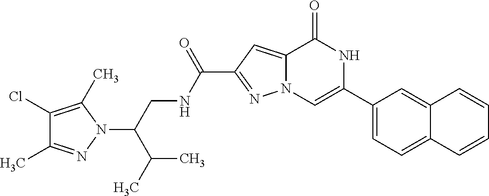 [SMILES: Cc1nn(C(CNC(=O)c2cc3c(=O)[nH]c(-c4ccc5ccccc5c4)cn3n2)C(C)C)c(C)c1Cl] | (ESIpos): m/z = 504 [M + H]⁺. |
| | (88% yield, 100% purity) | |
| 2-21 | 6-(2-Naphthyl)-4-oxo-N-[1-(1,3,5-trimethyl-1H-pyrazol- | LC/MS [Method 31]: |
| | 4-yl)ethyl]-4,5-dihydropyrazolo[1,5-a]pyrazine- | Rₜ = 1.06 min; MS |
| | 2-carboxamide 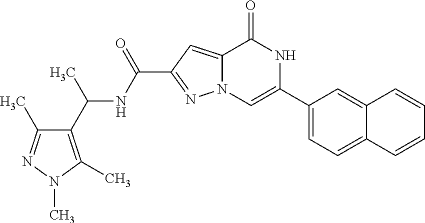 [SMILES: Cc1nn(C)c(C)c1C(C)NC(=O)c1cc2c(=O)[nH]c(-c3ccc4ccccc4c3)cn2n1] | (ESIpos): m/z = 441 [M + H]⁺. |
| | (69% yield, 96% purity) | |
| 2-22 | N-[2-(4-Chlor-1H-pyrazol-1-yl)-3-methylbutyl]-6- | LC/MS [Method 31]: |
| | (2-naphthyl)-4-oxo-4,5-dihydropyrazolo[1,5-a]pyrazine- | Rₜ = 1.16 min; MS |
| | 2-carboxamide 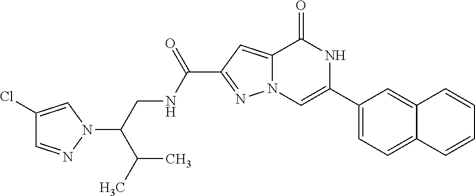 [SMILES: CC(C)C(CNC(=O)c1cc2c(=O)[nH]c(-c3ccc4ccccc4c3)cn2n1)n1cc(Cl)cn1] | (ESIpos): m/z = 475 [M + H]⁺. |
| | (67% yield, 100% purity) | |
| 2-23 | N-(3-Methoxybenzyl)-6-(2-naphthyl)-4-oxo-4,5- | LC/MS [Method 31]: |
| | dihydropyrazolo[1,5-a]pyrazine-2-carboxamide 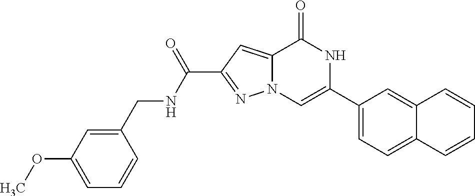 [SMILES: COc1cccc(CNC(=O)c2cc3c(=O)[nH]c(-c4ccc5ccccc5c4)cn3n2)c1] | Rₜ = 1.12 min; MS (ESIpos): m/z = 425 [M + H]⁺. |
| | (5% yield, 93% purity) | |
| 2-24 | N-(1H-Benzimidazol-2-ylmethyl)-6-(2-naphthyl)-4-oxo- | LC/MS [Method 31]: |
| | 4,5-dihydropyrazolo[1,5-a]pyrazine-2-carboxamide 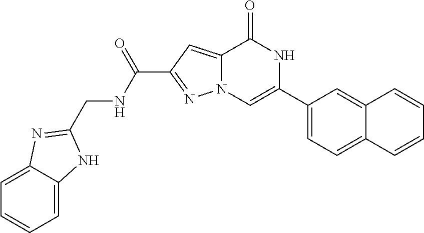 [SMILES: O=C(NCc1nc2ccccc2[nH]1)c1cc2c(=O)[nH]c(-c3ccc4ccccc4c3)cn2n1] | Rₜ = 0.79 min; MS (ESIpos): m/z = 435 [M + H]⁺. |
| | (8% yield, 93% purity) | |
| 2-25 | N-[(2S)-1-Methoxy-3-phenylpropan-2-yl]-6-(2- | LC/MS [Method 31]: |
| | naphthyl)-4-oxo-4,5-dihydropyrazolo[1,5-a]pyrazine-2- | Rₜ = 1.15 min; MS |
| | carboxamide 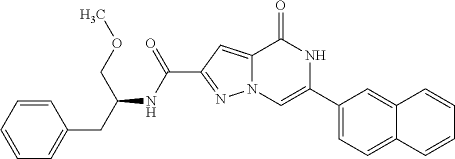 [SMILES: COC[C@H](Cc1ccccc1)NC(=O)c1cc2c(=O)[nH]c(-c3ccc4ccccc4c3)cn2n1] | (ESIpos): m/z = 453 [M + H]⁺. |
| | (9% yield, 100% purity) | |
| 2-26 | N-[1-(3,5-Dichlorophenyl)-2-hydroxyethyl]-6- | LC/MS [Method 31]: |
| | (2-naphthyl)-4-oxo-4,5-dihydropyrazolo[1,5-a]pyrazine- | Rₜ = 1.13 min; MS |
| | 2-carboxamide 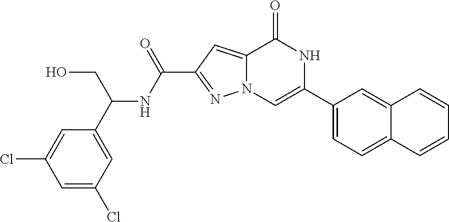 [SMILES: O=C(NC(CO)c1cc(Cl)cc(Cl)c1)c1cc2c(=O)[nH]c(-c3ccc4ccccc4c3)cn2n1] | (ESIpos): m/z = 494 [M + H]⁺. |
| | (5% yield, 98% purity) | |
| 2-27 | 6-(2-Naphthyl)-N-[(7R)-1-(1-naphthyl)ethyl]-4-oxo-4,5- | LC/MS [Method 31]: |
| | dihydropyrazolo[1,5-a]pyrazine-2-carboxamide 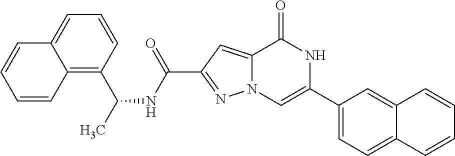 [SMILES: C[C@@H](NC(=O)c1cc2c(=O)[nH]c(-c3ccc4ccccc4c3)cn2n1)c1cccc2ccccc12] | Rₜ = 1.21 min; MS (ESIpos): m/z = 459 [M + H]⁺. |
| | (10% yield, 100% purity) | |
| 2-28 | N-[1-(1,3-Benzodioxol-5-yl)-2-hydroxyethyl]-6- | LC/MS [Method 31]: |
| | (2-naphthyl)-4-oxo-4,5-dihydropyrazolo[1,5-a]pyrazine- | Rₜ = 1.02 min; MS |
| | 2-carboxamide 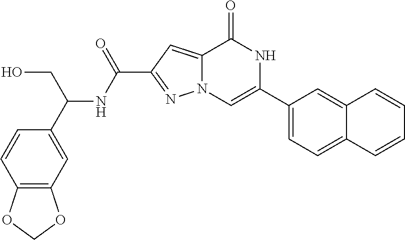 [SMILES: O=C(NC(CO)c1ccc2c(c1)OCO2)c1cc2c(=O)[nH]c(-c3ccc4ccccc4c3)cn2n1] | (ESIpos): m/z = 469 [M + H]⁺. |
| | (3% yield, 100% purity) | |
| 2-29 | N-[1-(4-Methyl-1H-pyrazol-1-yl)propan-2-yl]-6- | LC/MS [Method 31]: |
| | (2-naphthyl)-4-oxo-4,5-dihydropyrazolo[1,5-a]pyrazine- | Rₜ = 1.05 min; MS |
| | 2-carboxamide 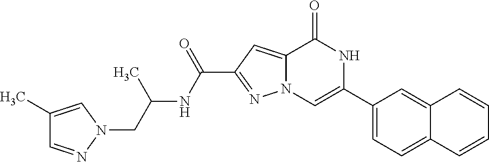 [SMILES: Cc1cnn(CC(C)NC(=O)c2cc3c(=O)[nH]c(-c4ccc5ccccc5c4)cn3n2)c1] | (ESIpos): m/z = 427 [M + H]⁺. |
| | (15% yield, 100% purity) | |
| 2-30 | N-[1-(2,6-Dimethylphenoxy)propan-2-yl]-6-(2- | LC/MS [Method 31]: |
| | naphthyl)-4-oxo-4,5-dihydropyrazolo[1,5-a]pyrazine-2- | Rₜ = 1.24 min; MS |
| | carboxamide 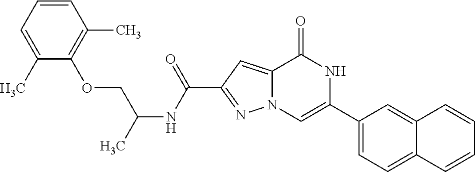 [SMILES: Cc1cccc(C)c1OCC(C)NC(=O)c1cc2c(=O)[nH]c(-c3ccc4ccccc4c3)cn2n1] | (ESIpos): m/z = 467 [M + H]⁺. |
| | (40% yield, 100% purity) | |
| 2-31 | N-(3,5-Difluorbenzyl)-6-(2-naphthyl)-4-oxo-4,5-dihydro- | LC/MS [Method 31]: |
| | pyrazolo[1,5-a]pyrazine-2-carboxamide 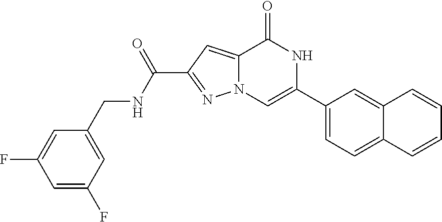 [SMILES: O=C(NCc1cc(F)cc(F)c1)c1cc2c(=O)[nH]c(-c3ccc4ccccc4c3)cn2n1] | Rₜ = 1.14 min; MS (ESIpos): m/z = 431 [M + H]⁺. |
| | (11% yield, 97% purity) | |
| 2-32 | 6-(2-Naphthyl)-4-oxo-N-[1,2,3,4-tetrahydronaphthalin- | LC/MS [Method 31]: |
| | 1-yl]-4,5-dihydropyrazolo[1,5-a]pyrazine-2-carbox- | Rₜ = 1.21 min; MS |
| | amide 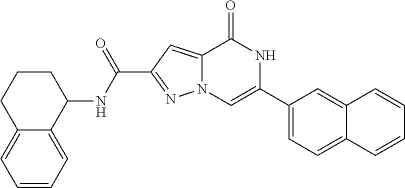 [SMILES: O=C(NC1CCCc2ccccc21)c1cc2c(=O)[nH]c(-c3ccc4ccccc4c3)cn2n1] | (ESIpos): m/z = 435 [M + H]⁺. |
| | (60% yield, 100% purity) | |
| 2-33 | N-(3,4-Difluorbenzyl)-6-(2-naphthyl)-4-oxo-4,5-dihydro- | LC/MS [Method 31]: |
| | pyrazolo[1,5-a]pyrazine-2-carboxamide 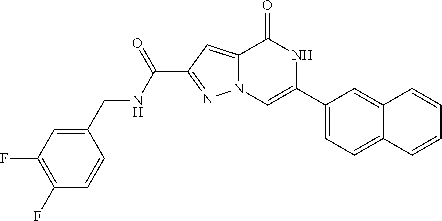 [SMILES: O=C(NCc1ccc(F)c(F)c1)c1cc2c(=O)[nH]c(-c3ccc4ccccc4c3)cn2n1] | Rₜ = 1.14 min; MS (ESIpos): m/z = 431 [M + H]⁺. |
| | (7% yield, 97% purity) | |
| 2-34 | 6-(2-Naphthyl)-4-oxo-N-[(7S)-1-phenylethyl]-4,5- | LC/MS [Method 31]: |
| | dihydropyrazolo[1,5-a]pyrazine-2-carboxamide 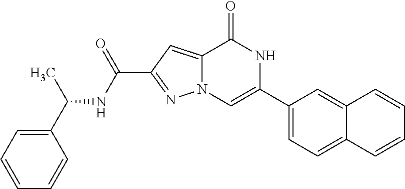 [SMILES: C[C@H](NC(=O)c1cc2c(=O)[nH]c(-c3ccc4ccccc4c3)cn2n1)c1ccccc1] | Rₜ = 1.14 min; MS (ESIpos): m/z = 409 [M + H]⁺. |
| | (33% yield, 93% purity) | |
| 2-35 | N-[1-(2,3-Dihydro-1,4-benzodioxin-6-yl)ethyl]-6- | LC/MS [Method 31]: |
| | (2-naphthyl)-4-oxo-4,5-dihydropyrazolo[1,5-a]pyrazine- | Rₜ = 1.13 min; MS |
| | 2-carboxamide 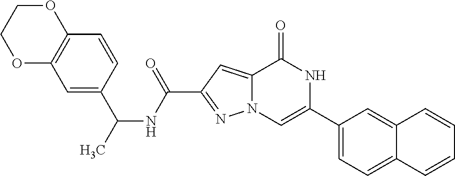 [SMILES: CC(NC(=O)c1cc2c(=O)[nH]c(-c3ccc4ccccc4c3)cn2n1)c1ccc2c(c1)OCCO2] | (ESIpos): m/z = 467 [M + H]⁺. |
| | (4% yield, 90% purity) | |
| 2-36 | N-[1-(3,4-Dichlorphenyl)ethyl]-6-(2-naphthyl)-4-oxo- | LC/MS [Method 31]: |
| | 4,5-dihydropyrazolo[1,5-a]pyrazine-2-carboxamide 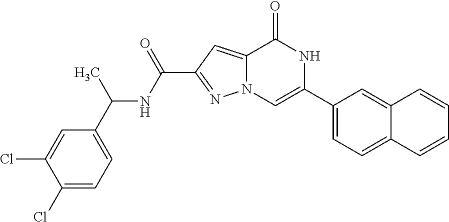 [SMILES: CC(NC(=O)c1cc2c(=O)[nH]c(-c3ccc4ccccc4c3)cn2n1)c1ccc(Cl)c(Cl)c1] | Rₜ = 1.22 min; MS (ESIpos): m/z = 478 [M + H]⁺. |
| | (7% yield, 96% purity) | |
| 2-37 | 6-(2-Naphthyl)-4-oxo-N-[1-(4-sulfamoylphenyl)ethyl]- | LC/MS [Method 31]: |
| | 4,5-dihydropyrazolo[1,5-a]pyrazine-2-carboxamide 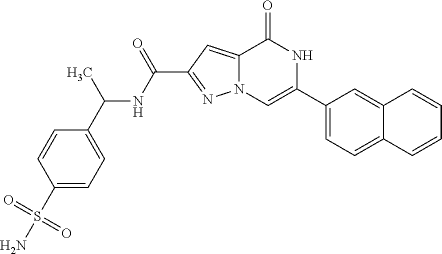 [SMILES: CC(NC(=O)c1cc2c(=O)[nH]c(-c3ccc4ccccc4c3)cn2n1)c1ccc(S(N)(=O)=O)cc1] | Rₜ = 1.00 min; MS (ESIpos): m/z = 488 [M + H]⁺. |
| | (77% yield, 97% purity) | |
| 2-38 | N-[2-Methyl-1-(2-thienyl)propyl]-6-(2-naphthyl)-4-oxo- | LC/MS [Method 31]: |
| | 4,5-dihydropyrazolo[1,5-a]pyrazine-2-carboxamide 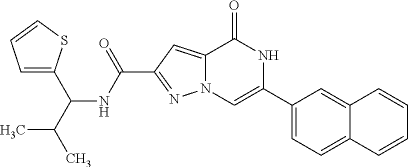 [SMILES: CC(C)C(NC(=O)c1cc2c(=O)[nH]c(-c3ccc4ccccc4c3)cn2n1)c1cccs1] | Rₜ = 1.20 min; MS (ESIpos): m/z = 443 [M + H]⁺. |
| | (74% yield, 100% purity) | |
| 2-39 | N-[1-(4-Ethylphenyl)ethyl]-6-(2-naphthyl)-4-oxo- | LC/MS [Method 31]: |
| | 4,5-dihydropyrazolo[1,5-a]pyrazine-2-carboxamide 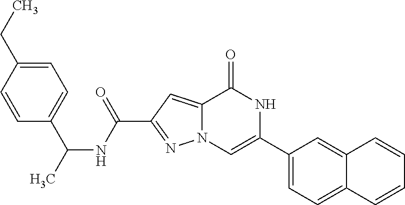 [SMILES: CCc1ccc(C(C)NC(=O)c2cc3c(=O)[nH]c(-c4ccc5ccccc5c4)cn3n2)cc1] | Rₜ = 1.22 min; MS (ESIpos): m/z = 437 [M + H]⁺. |
| | (65% yield, 95% purity) | |
| 2-40 | N-[1-(4-tert.-Butylphenyl)ethyl]-6-(2-naphthyl)-4-oxo- | LC/MS [Method 31]: |
| | 4,5-dihydropyrazolo[1,5-a]pyrazine-2-carboxamide 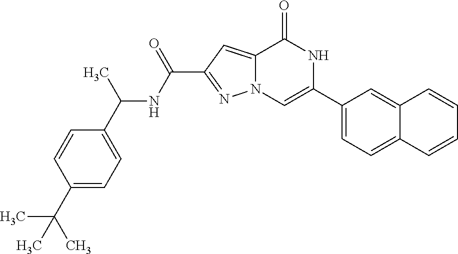 [SMILES: CC(NC(=O)c1cc2c(=O)[nH]c(-c3ccc4ccccc4c3)cn2n1)c1ccc(C(C)(C)C)cc1] | Rₜ = 1.26 min; MS (ESIpos): m/z = 465 [M + H]⁺. |
| | (65% yield, 95% purity) | |
| 2-41 | 6-(2-Naphthyl)-N-[(7R)-1-(2-naphthyl)ethyl]-4-oxo- | LC/MS [Method 31]: |
| | 4,5-dihydropyrazolo[1,5-a]pyrazine-2-carboxamide 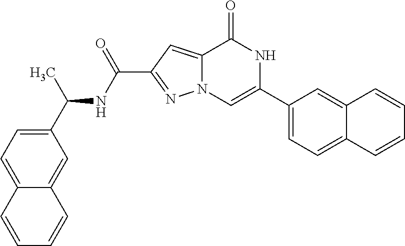 [SMILES: C[C@@H](NC(=O)c1cc2c(=O)[nH]c(-c3ccc4ccccc4c3)cn2n1)c1ccc2ccccc2c1] | Rₜ = 1.21 min; MS (ESIpos): m/z = 459 [M + H]⁺. |
| | (16% yield, 92% purity) | |
| 2-42 | N-[(7S)-2,3-Dihydro-1H-inden-1-yl]-6-(2-naphthyl)-4- | LC/MS [Method 31]: |
| | oxo-4,5-dihydropyrazolo[1,5-a]pyrazine-2-carboxamide 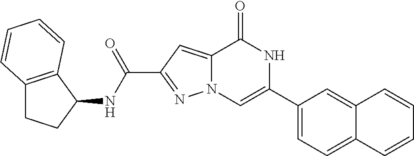 [SMILES: O=C(N[C@H]1CCc2ccccc21)c1cc2c(=O)[nH]c(-c3ccc4ccccc4c3)cn2n1] | Rₜ = 1.18 min; MS (ESIpos): m/z = 421 [M + H]⁺. |
| | (14% yield, 94% purity) | |
| 2-43 | 6-(2-Naphthyl)-4-oxo-N-[(7R)-1,2,3,4-tetrahydro- | LC/MS [Method 31]: |
| | naphthalin-1-yl]-4,5-dihydropyrazolo[1,5-a]pyrazine- | Rₜ = 1.21 min; MS |
| | 2-carboxamide 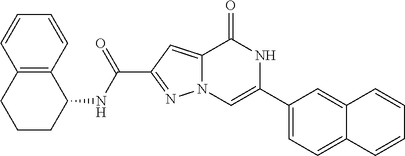 [SMILES: O=C(N[C@@H]1CCCc2ccccc21)c1cc2c(=O)[nH]c(-c3ccc4ccccc4c3)cn2n1] | (ESIpos): m/z = 435 [M + H]⁺. |
| | (65% yield, 98% purity) | |
| 2-44 | N-[1-(4-Methylphenyl)ethyl]-6-(2-naphthyl)-4-oxo- | LC/MS [Method 31]: |
| | 4,5-dihydropyrazolo[1,5-a]pyrazine-2-carboxamide 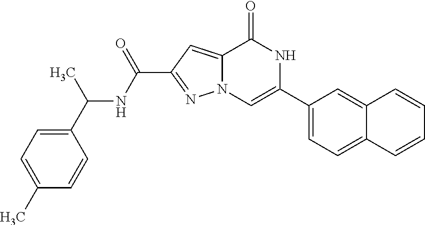 [SMILES: Cc1ccc(C(C)NC(=O)c2cc3c(=O)[nH]c(-c4ccc5ccccc5c4)cn3n2)cc1] | Rₜ = 1.18 min; MS (ESIpos): m/z = 423 [M + H]⁺. |
| | (68% yield, 97% purity) | |
| 2-45 | N-(Chinolin-5-yl)-6-(2-naphthyl)-4-oxo-4,5-dihydropyr- | LC/MS [Method 31]: |
| | azolo[1,5-a]pyrazine-2-carboxamide 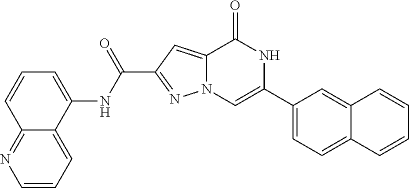 [SMILES: O=C(Nc1cccc2ncccc12)c1cc2c(=O)[nH]c(-c3ccc4ccccc4c3)cn2n1] | Rₜ = 1.02 min; MS (ESIpos): m/z = 432 [M + H]⁺. |
| | (23% yield, 98% purity) | |
| 2-46 | N-[2-(4-Chlor-3,5-dimethyl-1H-pyrazol-1-yl)propyl]-6- | LC/MS [Method 31]: |
| | (2-naphthyl)-4-oxo-4,5-dihydropyrazolo[1,5-a]pyrazine- | Rₜ = 1.14 min; MS |
| | 2-carboxamide 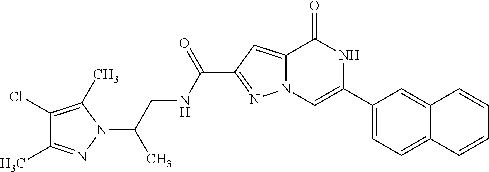 [SMILES: Cc1nn(C(C)CNC(=O)c2cc3c(=O)[nH]c(-c4ccc5ccccc5c4)cn3n2)c(C)c1Cl] | (ESIpos): m/z = 475 [M + H]⁺. |
| | (47% yield, 98% purity) | |
| 2-47 | N-[1-(2,4-Dichlorphenyl)ethyl]-6-(2-naphthyl)-4-oxo- | LC/MS [Method 31]: |
| | 4,5-dihydropyrazolo[1,5-a]pyrazine-2-carboxamide 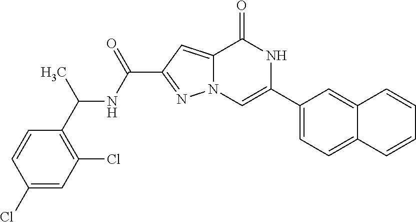 [SMILES: CC(NC(=O)c1cc2c(=O)[nH]c(-c3ccc4ccccc4c3)cn2n1)c1ccc(Cl)cc1Cl] | Rₜ = 1.23 min; MS (ESIpos): m/z = 478 [M + H]⁺. |
| | (6% yield, 93% purity) | |
| 2-48 | N-[1-(4-Fluorophenyl)-2-methylpropyl]-6-(2-naphthyl)- | LC/MS [Method 31]: |
| | 4-oxo-4,5-dihydropyrazolo[1,5-a]pyrazine-2- | Rₜ = 1.21 min; MS |
| | carboxamide 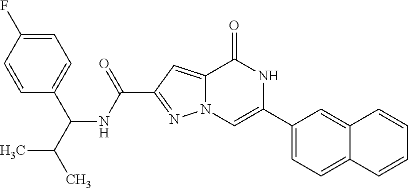 [SMILES: CC(C)C(NC(=O)c1cc2c(=O)[nH]c(-c3ccc4ccccc4c3)cn2n1)c1ccc(F)cc1] | (ESIpos): m/z = 455 [M + H]⁺. |
| | (34% yield, 92% purity) | |
| 2-49 | N-[1-(1-Methyl-1H-pyrazol-3-yl)ethyl]-6-(2-naphthyl)-4- | LC/MS [Method 31]: |
| | oxo-4,5-dihydropyrazolo[1,5-a]pyrazine-2-carboxamide 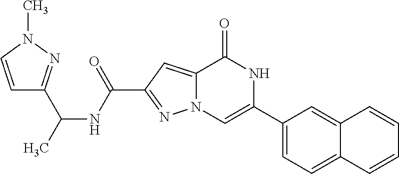 [SMILES: CC(NC(=O)c1cc2c(=O)[nH]c(-c3ccc4ccccc4c3)cn2n1)c1ccn(C)n1] | Rₜ = 1.02 min; MS (ESIpos): m/z = 413 [M + H]⁺. |
| | (84% yield, 96% purity) | |
| 2-50 | N-{1-[2-(Difluormethoxy)phenyl]ethyl}-6-(2-naphthyl)- | LC/MS [Method 31]: |
| | 4-oxo-4,5-dihydropyrazolo[1,5-a]pyrazine-2-carbox- | Rₜ = 1.18 min; MS |
| | amide 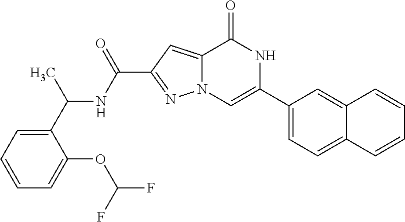 [SMILES: CC(NC(=O)c1cc2c(=O)[nH]c(-c3ccc4ccccc4c3)cn2n1)c1ccccc1OC(F)F] | (ESIpos): m/z = 475 [M + H]⁺. |
| | (58% yield, 94% purity) | |
| 2-51 | N-[1-(3,4-Dichlorphenyl)ethyl]-6-(2-naphthyl)-4-oxo- | LC/MS [Method 31]: |
| | 4,5-dihydropyrazolo[1,5-a]pyrazine-2-carboxamide 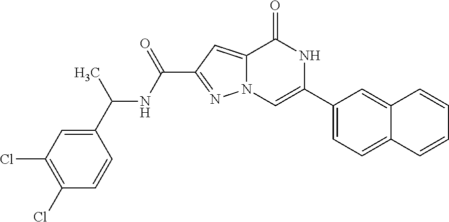 [SMILES: CC(NC(=O)c1cc2c(=O)[nH]c(-c3ccc4ccccc4c3)cn2n1)c1ccc(Cl)c(Cl)c1] | Rₜ = 1.22 min; MS (ESIpos): m/z = 478 [M + H]⁺. |
| | (8% yield, 95% purity) | |
| 2-52 | N-[2-(3-Fluorophenyl)ethyl]-6-(2-naphthyl)-4-oxo-4,5- | LC/MS [Method 31]: |
| | dihydropyrazolo[1,5-a]pyrazine-2-carboxamide 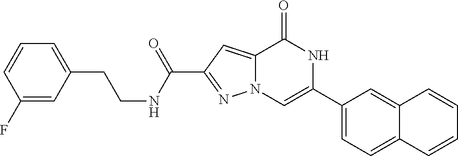 [SMILES: O=C(NCCc1cccc(F)c1)c1cc2c(=O)[nH]c(-c3ccc4ccccc4c3)cn2n1] | Rₜ = 1.16 min; MS (ESIpos): m/z = 427 [M + H]⁺. |
| | (27% yield, 98% purity) | |
| 2-53 | N-(2,5-Difluorobenzyl)-6-(2-naphthyl)-4-oxo-4,5- | LC/MS [Method 31]: |
| | dihydropyrazolo[1,5-a]pyrazine-2-carboxamide 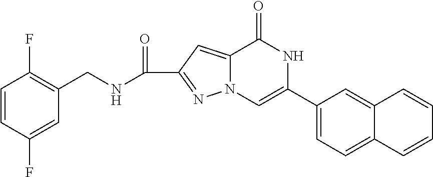 [SMILES: O=C(NCc1cc(F)ccc1F)c1cc2c(=O)[nH]c(-c3ccc4ccccc4c3)cn2n1] | Rₜ = 1.14 min; MS (ESIpos): m/z = 431 [M + H]⁺. |
| | (37% yield, 100% purity) | |
| 2-54 | N-[1-(1-Ethyl-1H-pyrazol-3-yl)ethyl]-6-(2-naphthyl)-4- | LC/MS [Method 31]: |
| | oxo-4,5-dihydropyrazolo[1,5-a]pyrazine-2-carboxamide 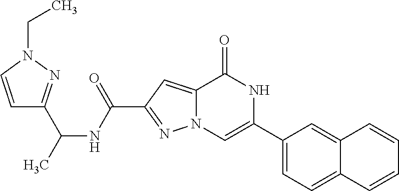 [SMILES: CCn1ccc(C(C)NC(=O)c2cc3c(=O)[nH]c(-c4ccc5ccccc5c4)cn3n2)n1] | Rₜ = 1.05 min; MS (ESIpos): m/z = 427 [M + H]⁺. |
| | (31% yield, 98% purity) | |
| 2-55 | N-[1-(3,4-Dimethoxyphenyl)ethyl]-6-(2-naphthyl)-4- | LC/MS [Method 31]: |
| | oxo-4,5-dihydropyrazolo[1,5-a]pyrazine-2-carboxamide 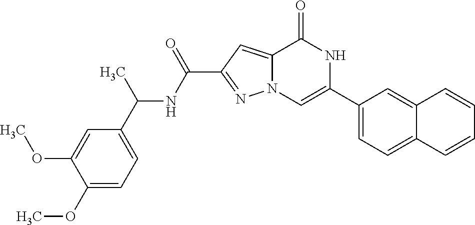 [SMILES: COc1ccc(C(C)NC(=O)c2cc3c(=O)[nH]c(-c4ccc5ccccc5c4)cn3n2)cc1OC] | Rₜ = 1.10 min; MS (ESIpos): m/z = 469 [M + H]⁺. |
| | (43% yield, 97% purity) | |

General Procedure 3 (Parallel Synthesis of Example Compounds):

The corresponding amine was placed on a multi-well plate, and a solution of 3,7-dimethyl-6-(2-naphthyl)-4-oxo-4,5-dihydropyrazolo[1,5-a]pyrazine-2-carboxylic acid (Intermediate 57A, 33.3 mg, 100 μmol) or 3-cyano-6-(3,4-dimethylphenyl)-4-oxo-4,5-dihydropyrazolo[1,5-a]pyrazine-2-carboxylic acid (Intermediate 108A, 33.0 mg, 100 μmol) in DMF (400 pl) was added, followed by a solution of HATU (49.4 mg, 130 μmol) in DMF (400 pl). Then, N-methylmorpholine (50 μl, 393 μmol) was added, and the plate was shaken at 40° C. overnight. After this, the reaction mixture was filtered, and the filtrate solution was directly purified by preparative HPLC (Method P11). The fractions containing the desired product were combined, evaporated and dried in vacuo.

The example compounds specified in the following Table 2 were prepared according to general procedure 3:

TABLE 2

| | IUPAC name/structure | |
|---|---|---|
| Example | (yield, purity) | Analytical data |
| 3-1 | N-[2-(3-Cyclopropyl-1H-pyrazol-1 -yl)ethyl]-3,7- | LC/MS [Method 31]: |
| | dimethyl-6-(naphthalen-2-yl)-4-oxo-4,5-dihydropyr- | Rₜ = 1.16 min; MS |
| | azolo[1,5-a]pyrazine-2-carboxamide 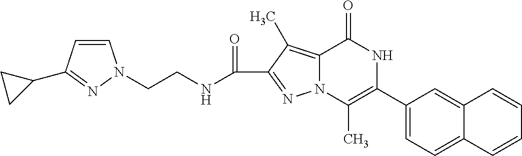 [SMILES: Cc1c(C(=O)NCCn2ccc(C3CC3)n2)nn2c(C)c(-c3ccc4ccccc4c3)[nH]c(=O)c12] | (ESIpos): m/z = 467 [M + H]⁺. |
| | (25% of theory, 90% purity) | |
| 3-2 | N-[(1S)-2-Hydroxy-1-phenylethyl]-3,7-dimethyl-6- | LC/MS [Method 31]: |
| | (naphthalen-2-yl)-4-oxo-4,5-dihydropyrazolo[1,5-a]- | Rₜ = 1.12 min; MS |
| | pyrazine-2-carboxamide 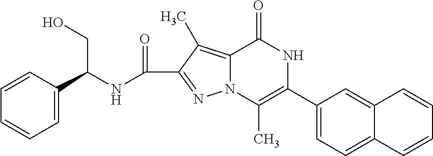 [SMILES: Cc1c(C(=O)N[C@H](CO)c2ccccc2)nn2c(C)c(-c3ccc4ccccc4c3)[nH]c(=O)c12] | (ESIpos): m/z = 453 [M + H]⁺. |
| | (33% of theory, 100% purity) | |
| 3-3 | 3,7-Dimethyl-6-(naphthalen-2-yl)-4-oxo-N-[2-(2,2,2-tri- | LC/MS [Method 31]: |
| | fluoroethoxy)ethyl]-4,5-dihydropyrazolo[1,5-a]pyrazine- | Rₜ = 1.18 min; MS |
| | 2-carboxamide 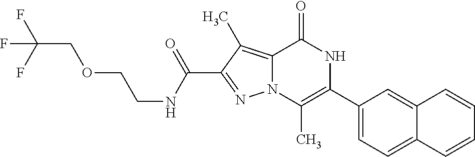 [SMILES: Cc1c(C(=O)NCCOCC(F)(F)F)nn2c(C)c(-c3ccc4ccccc4c3)[nH]c(=O)c12] | (ESIpos): m/z = 459 [M + H]⁺. |
| | (11% of theory, 100% purity) | |
| 3-4 | N-[1-(1-Ethylpyrazol-3-yl)ethyl]-3,7-dimethyl-6-(2-naph- | LC/MS [Method 31]: |
| | thyl)-4-oxo-5H-pyrazolo[1,5-a]pyrazine-2-carboxamide 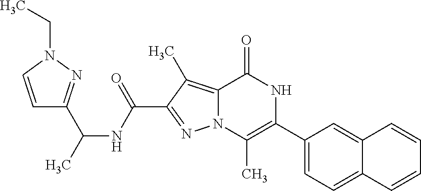 [SMILES: CCn1ccc(C(C)NC(=O)c2nn3c(C)c(-c4ccc5ccccc5c4)[nH]c(=O)c3c2C)n1] | Rₜ = 1.15 min; MS (ESIpos): m/z = 455 [M + H]⁺. |
| | (18% of theory, 92% purity) | |
| 3-5 | 3,7-Dimethyl-6-(naphthalen-2-yl)-4-oxo-N-[(1H- | LC/MS [Method 31]: |
| | pyrazol-3-yl)methyl]-4,5-dihydropyrazolo[1,5-a]- | Rₜ = 1.03 min; MS |
| | pyrazine-2-carboxamide 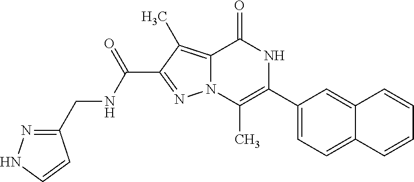 [SMILES: Cc1c(C(=O)NCc2cc[nH]n2)nn2c(C)c(-c3ccc4ccccc4c3)[nH]c(=O)c12] | (ESIpos): m/z = 413 [M + H]⁺. |
| | (19% of theory, 100% purity) | |
| 3-6 | 3,7-Dimethyl-6-(naphthalen-2-yl)-4-oxo-N-[1-(propan- | LC/MS [Method 31]: |
| | 2-yl)-1H-pyrazol-5-yl]-4,5-dihydropyrazolo[1,5-a]- | Rₜ = 1.16 min; MS |
| | pyrazine-2-carboxamide 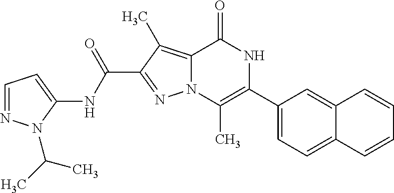 [SMILES: Cc1c(C(=O)Nc2ccnn2C(C)C)nn2c(C)c(-c3ccc4ccccc4c3)[nH]c(=O)c12] | (ESIpos): m/z = 441 [M + H]⁺. |
| | (35% of theory, 100% purity) | |
| 3-7 | 3,7-Dimethyl-N-[2-methyl-3-(1H-pyrazol-1-yl)propyl]-6- | LC/MS [Method 31]: |
| | (2-naphthyl)-4-oxo-4,5-dihydropyrazolo[1,5-a]pyrazine- | Rₜ = 1.14 min; MS |
| | 2-carboxamide 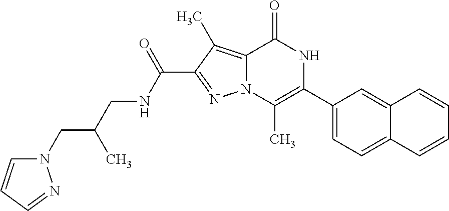 [SMILES: Cc1c(C(=O)NCC(C)Cn2cccn2)nn2c(C)c(-c3ccc4ccccc4c3)[nH]c(=O)c12] | (ESIpos): m/z = 455 [M + H]⁺. |
| | (8% of theory, 100% purity) | |
| 3-8 | N-[(4-Chloro-1-ethyl-1H-pyrazol-3-yl)methyl]-3,7- | LC/MS [Method 31]: |
| | dimethyl-6-(naphthalen-2-yl)-4-oxo-4,5-dihydropyr- | Rₜ = 1.19 min; MS |
| | azolo[1,5-a]pyrazine-2-carboxamide 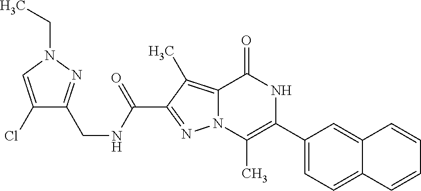 [SMILES: CCn1cc(Cl)c(CNC(=O)c2nn3c(C)c(-c4ccc5ccccc5c4)[nH]c(=O)c3c2C)n1] | (ESIpos): m/z = 475 [M + H]⁺. |
| | (4% of theory, 100% purity) | |
| 3-9 | N-[(1,3-Dimethyl-1H-pyrazol-4-yl)methyl]-3,7-dimethyl- | LC/MS [Method 31]: |
| | 6-(naphthalen-2-yl)-4-oxo-4,5-dihydropyrazolo[1,5-a]- | Rₜ = 1.14 min; MS |
| | pyrazine-2-carboxamide 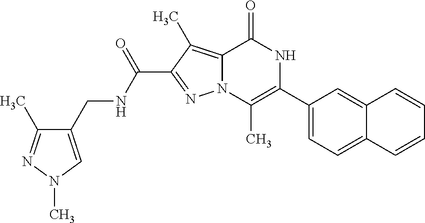 [SMILES: Cc1nn(C)cc1CNC(=O)c1nn2c(C)c(-c3ccc4ccccc4c3)[nH]c(=O)c2c1C] | (ESIpos): m/z = 441 [M + H]⁺. |
| | (15% of theory, 100% purity) | |
| 3-10 | N-(1-Cyclopropylpropan-2-yl)-3,7-dimethyl-6- | LC/MS [Method 31]: |
| | (2-naphthyl)-4-oxo-4,5-dihydropyrazolo[1,5-a]pyrazine- | Rₜ = 1.25 min; MS |
| | 2-carboxamide 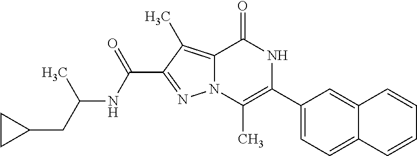 [SMILES: Cc1c(C(=O)NC(C)CC2CC2)nn2c(C)c(-c3ccc4ccccc4c3)[nH]c(=O)c12] | (ESIpos): m/z = 415 [M + H]⁺. |
| | (11% of theory, 100% purity) | |
| 3-11 | 3,7-Dimethyl-N-[(1-methyl-1H-pyrazol-5-yl)methyl]- | LC/MS [Method 31]: |
| | 6-(naphthalen-2-yl)-4-oxo-4,5-dihydropyrazolo[1,5-a]- | Rₜ = 1.12 min; MS |
| | pyrazine-2-carboxamide 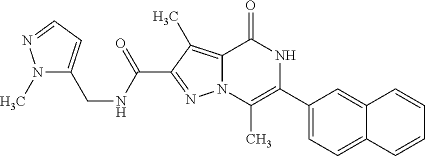 [SMILES: Cc1c(C(=O)NCc2ccnn2C)nn2c(C)c(-c3ccc4ccccc4c3)[nH]c(=O)c12] | (ESIpos): m/z = 427 [M + H]⁺. |
| | (12% of theory, 91% purity) | |
| 3-12 | N-[1-(1-Ethyl-1H-pyrazol-5-yl)ethyl]-3,7-dimethyl-6- | LC/MS [Method 31]: |
| | (2-naphthyl)-4-oxo-4,5-dihydropyrazolo[1,5-a]pyrazine- | Rₜ = 1.17 min; MS |
| | 2-carboxamide 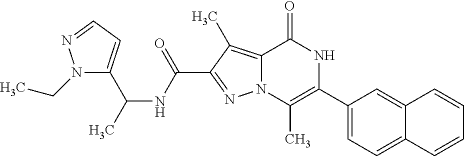 [SMILES: CCn1nccc1C(C)NC(=O)c1nn2c(C)c(-c3ccc4ccccc4c3)[nH]c(=O)c2c1C] | (ESIpos): m/z = 455 [M + H]⁺. |
| | (2% of theory, 100% purity) | |
| 3-13 | N-(2,3-Dihydro-1H-inden-2-yl)-3,7-dimethyl-6- | LC/MS [Method 31]: |
| | (naphthalen-2-yl)-4-oxo-4,5-dihydropyrazolo[1,5-a]- | Rₜ = 1.26 min; MS |
| | pyrazine-2-carboxamide 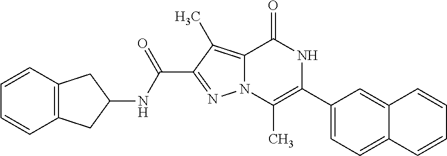 [SMILES: Cc1c(C(=O)NC2Cc3ccccc3C2)nn2c(C)c(-c3ccc4ccccc4c3)[nH]c(=O)c12] | (ESIpos): m/z = 449 [M + H]⁺. |
| | (2% of theory, 100% purity) | |
| 3-14 | N-[(1-Ethyl-3-methyl-1H-pyrazol-4-yl)methyl]-3,7- | LC/MS [Method 31]: |
| | dimethyl-6-(naphthalen-2-yl)-4-oxo-4,5-dihydropyr- | Rₜ = 1.13 min; MS |
| | azolo[1,5-a]pyrazine-2-carboxamide 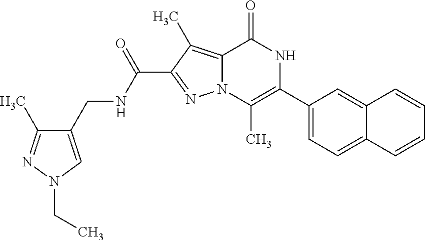 [SMILES: CCn1cc(CNC(=O)c2nn3c(C)c(-c4ccc5ccccc5c4)[nH]c(=O)c3c2C)c(C)n1] | (ESIpos): m/z = 455 [M + H]⁺. |
| | (16% of theory, 100% purity) | |
| 3-15 | 3,7-Dimethyl-N-[(3-methyl-1,2-oxazol-5-yl)methyl]-6- | LC/MS [Method 31]: |
| | (naphthalen-2-yl)-4-oxo-4,5-dihydropyrazolo[1,5-a]- | Rₜ = 1.13 min; MS |
| | pyrazine-2-carboxamide 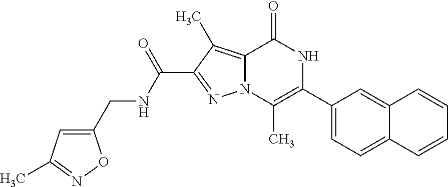 [SMILES: Cc1cc(CNC(=O)c2nn3c(C)c(-c4ccc5ccccc5c4)[nH]c(=O)c3c2C)on1] | (ESIpos): m/z = 428 [M + H]⁺. |
| | (19% of theory, 100% purity) | |
| 3-16 | N-[1-(3,4-Dichlorophenyl)ethyl]-3,7-dimethyl-6-(2-naph- | LC/MS [Method 31]: |
| | thyl)-4-oxo-5H-pyrazolo[1,5-a]pyrazine-2-carboxamide 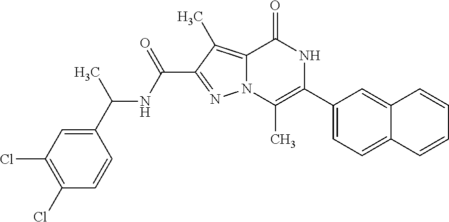 [SMILES: Cc1c(C(=O)NC(C)c2ccc(Cl)c(Cl)c2)nn2c(C)c(-c3ccc4ccccc4c3)[nH]c(=O)c12] | Rₜ = 1.30 min; MS (ESIpos): m/z = 505 [M + H]⁺. |
| | (5% of theory, 99% purity) | |
| 3-17 | 3,7-Dimethyl-6-(2-naphthyl)-4-oxo-N-[1-(2-pyridyl)- | LC/MS [Method 31]: |
| | ethyl]-5H-pyrazolo[1,5-a]pyrazine-2-carboxamide 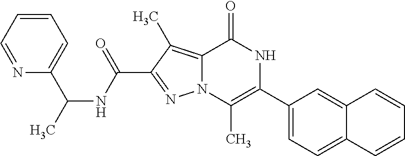 [SMILES: Cc1c(C(=O)NC(C)c2ccccn2)nn2c(C)c(-c3ccc4ccccc4c3)[nH]c(=O)c12] | Rₜ = 1.07 min; MS (ESIpos): m/z = 438 [M + H]⁺. |
| | (20% of theory, 100% purity) | |
| 3-18 | 3,7-Dimethyl-6-(2-naphthyl)-4-oxo-N-[1-(4-sulfamoyl- | LC/MS [Method 31]: |
| | phenyl)ethyl]-5H-pyrazolo[1,5-a]pyrazine-2-carbox- | Rₜ = 1.09 min; MS |
| | amide 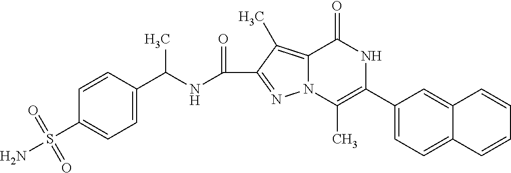 [SMILES: Cc1c(C(=O)NC(C)c2ccc(S(N)(=O)=O)cc2)nn2c(C)c(-c3ccc4ccccc4c3)[nH]c(=O)c12] | (ESIpos): m/z = 516 [M + H]⁺. |
| | (30% of theory, 92% purity) | |
| 3-19 | 3,7-Dimethyl-N-[(7R)-1-(4-methylphenyl)ethyl]-6- | LC/MS [Method 31]: |
| | (naphthalen-2-yl)-4-oxo-4,5-dihydropyrazolo[1,5-a]- | Rₜ = 1.27 min; MS |
| | pyrazine-2-carboxamide 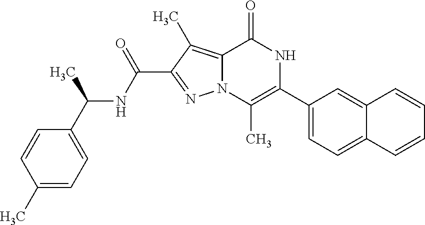 [SMILES: Cc1ccc([C@@H](C)NC(=O)c2nn3c(C)c(-c4ccc5ccccc5c4)[nH]c(=O)c3c2C)cc1] | (ESIpos): m/z = 451 [M + H]⁺. |
| | (16% of theory, 100% purity) | |
| 3-20 | 3,7-Dimethyl-N-[(7S)-1-(4-methylphenyl)ethyl]-6- | LC/MS [Method 31]: |
| | (naphthalen-2-yl)-4-oxo-4,5-dihydropyrazolo[1,5-a]- | Rₜ = 1.27 min; MS |
| | pyrazine-2-carboxamide 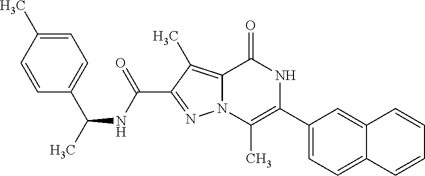 [SMILES: Cc1ccc([C@H](C)NC(=O)c2nn3c(C)c(-c4ccc5ccccc5c4)[nH]c(=O)c3c2C)cc1] | (ESIpos): m/z = 451 [M + H]⁺. |
| | (14% of theory, 99% purity) | |
| 3-21 | N-[1-(4-Fluorophenyl)ethyl]-3,7-dimethyl-6-(2- | LC/MS [Method 31]: |
| | naphthyl)-4-oxo-5H-pyrazolo[1,5-a]pyrazine-2- | Rₜ = 1.24 min; MS |
| | carboxamide 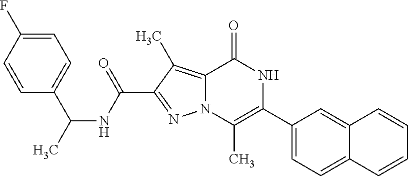 [SMILES: Cc1c(C(=O)NC(C)c2ccc(F)cc2)nn2c(C)c(-c3ccc4ccccc4c3)[nH]c(=O)c12] | (ESIpos): m/z = 455 [M + H]⁺. |
| | (2% of theory, 100% purity) | |
| 3-22 | 3,7-Dimethyl-6-(naphthalen-2-yl)-4-oxo-N-[(7S)-1- | LC/MS [Method 31]: |
| | phenylethyl]-4,5-dihydropyrazolo[1,5-a]pyrazine- | Rₜ = 1.24 min; MS |
| | 2-carboxamide 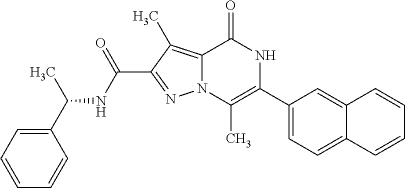 [SMILES: Cc1c(C(=O)N[C@@H](C)c2ccccc2)nn2c(C)c(-c3ccc4ccccc4c3)[nH]c(=O)c12] | (ESIpos): m/z = 437 [M + H]⁺. |
| | (11% of theory, 100% purity) | |
| 3-23 | 3,7-Dimethyl-N-[1-(4-methylsulfonylphenyl)ethyl]-6- | LC/MS [Method 31]: |
| | (2-naphthyl)-4-oxo-5H-pyrazolo[1,5-a]pyrazine- | Rₜ = 1.15 min; MS |
| | 2-carboxamide 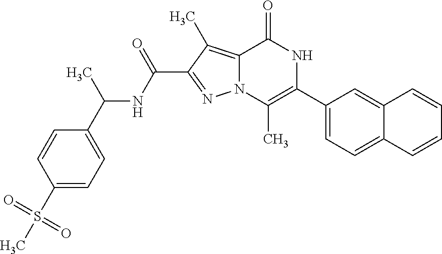 [SMILES: Cc1c(C(=O)NC(C)c2ccc(S(C)(=O)=O)cc2)nn2c(C)c(-c3ccc4ccccc4c3)[nH]c(=O)c12] | (ESIpos): m/z = 515 [M + H]⁺. |
| | (27% of theory, 98% purity) | |
| 3-24 | N-[1-(3-Bromophenyl)ethyl]-3,7-dimethyl-6-(2- | LC/MS [Method 31]: |
| | naphthyl)-4-oxo-5H-pyrazolo[1,5-a]pyrazine-2- | Rₜ = 1.28 min; MS |
| | carboxamide 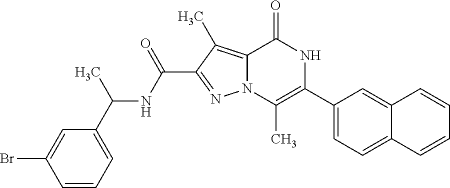 [SMILES: Cc1c(C(=O)NC(C)c2cccc(Br)c2)nn2c(C)c(-c3ccc4ccccc4c3)[nH]c(=O)c12] | (ESIpos): m/z = 515 [M + H]⁺. |
| | (23% of theory, 100% purity) | |
| 3-25 | 3,7-Dimethyl-6-(naphthalen-2-yl)-4-oxo-N-(1-phenyl- | LC/MS [Method 31]: |
| | cyclopropyl)-4,5-dihydropyrazolo[1,5-a]pyrazine- | Rₜ = 1.23 min; MS |
| | 2-carboxamide 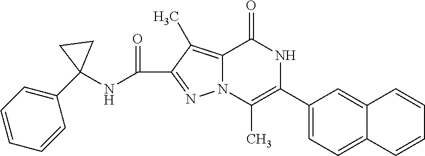 [SMILES: Cc1c(C(=O)NC2(c3ccccc3)CC2)nn2c(C)c(-c3ccc4ccccc4c3)[nH]c(=O)c12] | (ESIpos): m/z = 449 [M + H]⁺. |
| | (6% of theory, 100% purity) | |
| 3-26 | N-[1-(4-Ethylphenyl)ethyl]-3,7-dimethyl-6-(2-naphthyl)- | LC/MS [Method 31]: |
| | 4-oxo-5H-pyrazolo[1,5-a]pyrazine-2-carboxamide 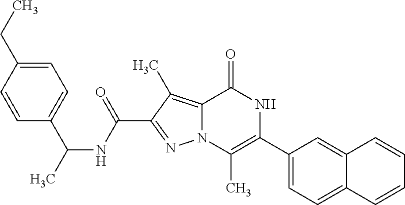 [SMILES: CCc1ccc(C(C)NC(=O)c2nn3c(C)c(-c4ccc5ccccc5c4)[nH]c(=O)c3c2C)cc1] | Rₜ = 1.29 min; MS (ESIpos): m/z = 465 [M + H]⁺. |
| | (18% of theory, 100% purity) | |
| 3-27 | N-[1-(4-Cyanophenyl)cyclopropyl]-3,7-dimethyl-6- | LC/MS [Method 31]: |
| | (naphthalen-2-yl)-4-oxo-4,5-dihydropyrazolo[1,5-a]- | Rₜ = 1.21 min; MS |
| | pyrazine-2-carboxamide 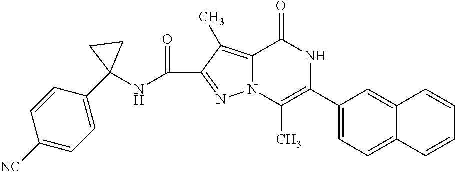 [SMILES: Cc1c(C(=O)NC2(c3ccc(C#N)cc3)CC2)nn2c(C)c(-c3ccc4ccccc4c3)[nH]c(=O)c12] | (ESIpos): m/z = 474 [M + H]⁺. |
| | (5% of theory, 100% purity) | |
| 3-28 | N-[1-(2-Methoxyphenyl)ethyl]-3,7-dimethyl-6-(2- | LC/MS [Method 31]: |
| | naphthyl)-4-oxo-5H-pyrazolo[1,5-a]pyrazine- | Rₜ = 1.27 min; MS |
| | 2-carboxamide 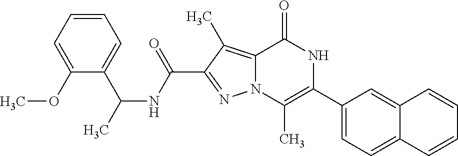 [SMILES: COc1ccccc1C(C)NC(=O)c1nn2c(C)c(-c3ccc4ccccc4c3)[nH]c(=O)c2c1C] | (ESIpos): m/z = 467 [M + H]⁺. |
| | (6% of theory, 100% purity) | |
| 3-29 | N-[(7S)-2,3-Dihydro-1H-inden-1-yl]-3,7-dimethyl-6- | LC/MS [Method 31]: |
| | (naphthalen-2-yl)-4-oxo-4,5-dihydropyrazolo[1,5-a]- | Rₜ = 1.26 min; MS |
| | pyrazine-2-carboxamide 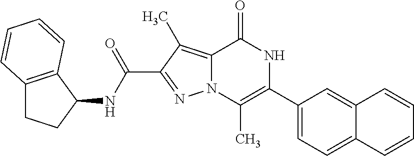 [SMILES: Cc1c(C(=O)N[C@H]2CCc3ccccc32)nn2c(C)c(-c3ccc4ccccc4c3)[nH]c(=O)c12] | (ESIpos): m/z = 449 [M + H]⁺. |
| | (3% of theory, 100% purity) | |
| 3-30 | N-[2-(3,5-Dichlorophenyl)propan-2-yl]-3,7-dimethyl-6- | LC/MS [Method 31]: |
| | (naphthalen-2-yl)-4-oxo-4,5-dihydropyrazolo[1,5-a]- | Rₜ = 1.33 min; MS |
| | pyrazine-2-carboxamide 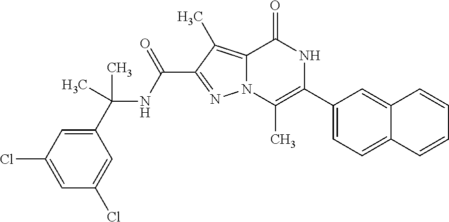 [SMILES: Cc1c(C(=O)NC(C)(C)c2cc(Cl)cc(Cl)c2)nn2c(C)c(-c3ccc4ccccc4c3)[nH]c(=O)c12] | (ESIpos): m/z = 519 [M + H]⁺. |
| | (30% of theory, 99% purity) | |
| 3-31 | N-[1-(2,3-Dihydro-1,4-benzodioxin-6-yl)ethyl]-3,7- | LC/MS [Method 31]: |
| | dimethyl-6-(2-naphthyl)-4-oxo-5H-pyrazolo[1,5-a]- | Rₜ = 1.23 min; MS |
| | pyrazine-2-carboxamide 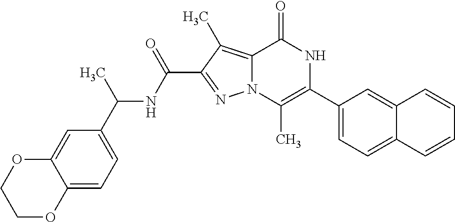 [SMILES: Cc1c(C(=O)NC(C)c2ccc3c(c2)OCCO3)nn2c(C)c(-c3ccc4ccccc4c3)[nH]c(=O)c12] | (ESIpos): m/z = 495 [M + H]⁺. |
| | (13% of theory, 100% purity) | |
| 3-32 | N-[1-(4-Chlorophenyl)cyclobutyl]-3,7-dimethyl-6- | LC/MS [Method 31]: |
| | (naphthalen-2-yl)-4-oxo-4,5-dihydropyrazolo[1,5-a]- | Rₜ = 1.30 min; MS |
| | pyrazine-2-carboxamide 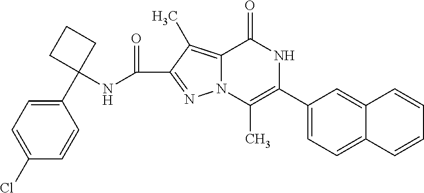 [SMILES: Cc1c(C(=O)NC2(c3ccc(Cl)cc3)CCC2)nn2c(C)c(-c3ccc4ccccc4c3)[nH]c(=O)c12] | (ESIpos): m/z = 497 [M + H]⁺. |
| | (10% of theory, 100% purity) | |
| 3-33 | 3,7-Dimethyl-6-(naphthalen-2-yl)-4-oxo-N-[(7R)-1- | LC/MS [Method 31]: |
| | phenylpropyl]-4,5-dihydropyrazolo[1,5-a]pyrazine- | Rₜ = 1.26 min; MS |
| | 2-carboxamide 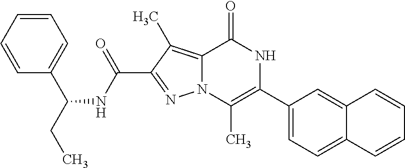 [SMILES: CC[C@@H](NC(=O)c1nn2c(C)c(-c3ccc4ccccc4c3)[nH]c(=O)c2c1C)c1ccccc1] | (ESIpos): m/z = 451 [M + H]⁺. |
| | (7% of theory, 100% purity) | |
| 3-34 | 3,7-Dimethyl-6-(2-naphthyl)-4-oxo-N-(4-phenyltetra- | LC/MS [Method 31]: |
| | hydro-2H-pyran-4-yl)-4,5-dihydropyrazolo[1,5-a]- | Rₜ = 1.24 min; MS |
| | pyrazine-2-carboxamide 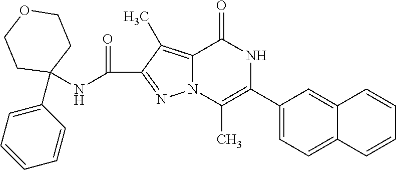 [SMILES: Cc1c(C(=O)NC2(c3ccccc3)CCOCC2)nn2c(C)c(-c3ccc4ccccc4c3)[nH]c(=O)c12] | (ESIpos): m/z = 493 [M + H]⁺. |
| | (1% of theory, 100% purity) | |
| 3-35 | N-[Cyclopropyl(4-methoxyphenyl)methyl]-3,7-dimethyl- | LC/MS [Method 31]: |
| | 6-(2-naphthyl)-4-oxo-5H-pyrazolo[1,5-a]pyrazine- | Rₜ = 1.25 min; MS |
| | 2-carboxamide 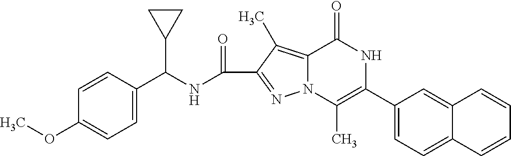 [SMILES: COc1ccc(C(NC(=O)c2nn3c(C)c(-c4ccc5ccccc5c4)[nH]c(=O)c3c2C)C2CC2)cc1] | (ESIpos): m/z = 493 [M + H]⁺. |
| | (17% of theory, 100% purity) | |
| 3-36 | 3,7-Dimethyl-6-(naphthalen-2-yl)-4-oxo-N-({3- | LC/MS [Method 31]: |
| | [(pyrrolidin-1-yl)methyl]phenyl}methyl)-4,5-dihydropyr- | Rₜ = 0.84 min; MS |
| | azolo[1,5-a]pyrazine-2-carboxamide 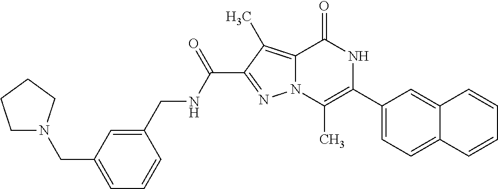 [SMILES: Cc1c(C(=O)NCc2cccc(CN3CCCC3)c2)nn2c(C)c(-c3ccc4ccccc4c3)[nH]c(=O)c12] | (ESIpos): m/z = 506 [M + H]⁺. |
| | (11% of theory, 95% purity) | |
| 3-37 | N-[3-Hydroxy-1-(4-methoxyphenyl)propyl]-3,7- | LC/MS [Method 31]: |
| | dimethyl-6-(2-naphthyl)-4-oxo-5H-pyrazolo[1,5-a]- | Rₜ = 1.12 min; MS |
| | pyrazine-2-carboxamide 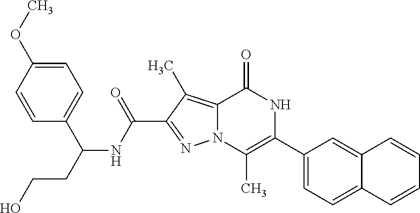 [SMILES: COc1ccc(C(CCO)NC(=O)c2nn3c(C)c(-c4ccc5ccccc5c4)[nH]c(=O)c3c2C)cc1] | (ESIpos): m/z = 497 [M + H]⁺. |
| | (19% of theory, 100% purity) | |
| 3-38 | N-[1-(6-Methoxy-3-pyridyl)ethyl]-3,7-dimethyl-6- | LC/MS [Method 31]: |
| | (2-naphthyl)-4-oxo-5H-pyrazolo[1,5-a]pyrazine- | Rₜ = 1.19 min; MS |
| | 2-carboxamide 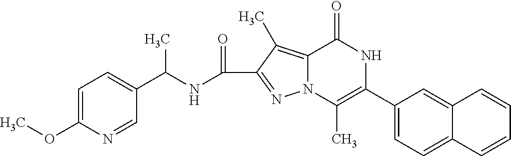 [SMILES: COc1ccc(C(C)NC(=O)c2nn3c(C)c(-c4ccc5ccccc5c4)[nH]c(=O)c3c2C)cn1] | (ESIpos): m/z = 468 [M + H]⁺. |
| | (25% of theory, 100% purity) | |
| 3-39 | N-[3-Hydroxy-1-(3-methoxyphenyl)propyl]-3,7- | LC/MS [Method 31]: |
| | dimethyl-6-(2-naphthyl)-4-oxo-5H-pyrazolo[1,5-a]- | Rₜ = 1.13 min; MS |
| | pyrazine-2-carboxamide 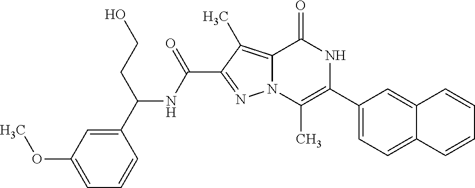 [SMILES: COc1cccc(C(CCO)NC(=O)c2nn3c(C)c(-c4ccc5ccccc5c4)[nH]c(=O)c3c2C)c1] | (ESIpos): m/z = 497 [M + H]⁺. |
| | (25% of theory, 100% purity) | |
| 3-40 | N-[1-(3,5-Dichlorophenyl)-2-hydroxyethyl]-3,7- | LC/MS [Method 31]: |
| | dimethyl-6-(2-naphthyl)-4-oxo-5H-pyrazolo[1,5-a]- | Rₜ = 1.21 min; MS |
| | pyrazine-2-carboxamide 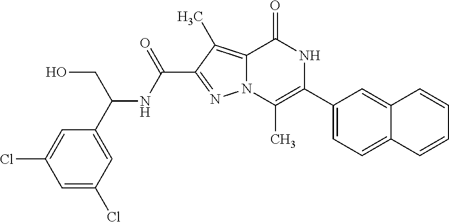 [SMILES: Cc1c(C(=O)NC(CO)c2cc(Cl)cc(Cl)c2)nn2c(C)c(-c3ccc4ccccc4c3)[nH]c(=O)c12] | (ESIpos): m/z = 521 [M + H]⁺. |
| | (16% of theory, 100% purity) | |
| 3-41 | N-[1-(4-Fluorophenyl)-2-methylpropyl]-3,7-dimethyl-6- | LC/MS [Method 31]: |
| | (2-naphthyl)-4-oxo-5H-pyrazolo[1,5-a]pyrazine- | Rₜ = 1.29 min; MS |
| | 2-carboxamide 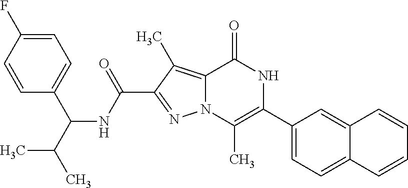 [SMILES: Cc1c(C(=O)NC(c2ccc(F)cc2)C(C)C)nn2c(C)c(-c3ccc4ccccc4c3)[nH]c(=O)c12] | (ESIpos): m/z = 483 [M + H]⁺. |
| | (6% of theory, 100% purity) | |
| 3-42 | N-({3-[(Dimethylamino)methyl]phenyl}methyl)-3,7- | LC/MS [Method 31]: |
| | dimethyl-6-(naphthalen-2-yl)-4-oxo-4,5-dihydropyr- | Rₜ = 0.82 min; MS |
| | azolo[1,5-a]pyrazine-2-carboxamide 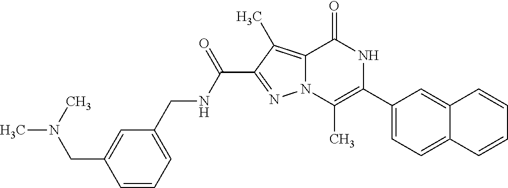 [SMILES: Cc1c(C(=O)NCc2cccc(CN(C)C)c2)nn2c(C)c(-c3ccc4ccccc4c3)[nH]c(=O)c12] | (ESIpos): m/z = 480 [M + H]⁺. |
| | (14% of theory, 100% purity) | |
| 3-43 | N-[1-[2-(Difluoromethoxy)phenyl]ethyl]-3,7-dimethyl- | LC/MS [Method 31]: |
| | 6-(2-naphthyl)-4-oxo-5H-pyrazolo[1,5-a]pyrazine- | Rₜ = 1.27 min; MS |
| | 2-carboxamide 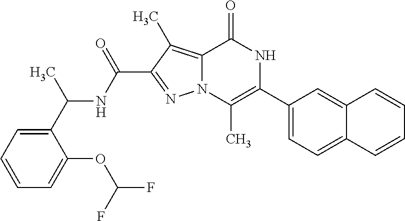 [SMILES: Cc1c(C(=O)NC(C)c2ccccc2OC(F)F)nn2c(C)c(-c3ccc4ccccc4c3)[nH]c(=O)c12] | (ESIpos): m/z = 503 [M + H]⁺. |
| | (25% of theory, 96% purity) | |
| 3-44 | 3-Cyano-N-[(1-ethyl-3-methyl-1H-pyrazol-4-yl)methyl]- | LC/MS [Method 31]: |
| | 6-(naphthalen-2-yl)-4-oxo-4,5-dihydropyrazolo[1,5-a]- | Rₜ = 1.06 min; MS |
| | pyrazine-2-carboxamide 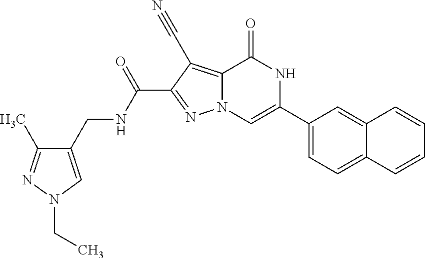 [SMILES: CCn1cc(CNC(=O)c2nn3cc(-c4ccc5ccccc5c4)[nH]c(=O)c3c2C#N)c(C)n1] | (ESIpos): m/z = 452 [M + H]⁺. |
| | (5% of theory, 100% purity) | |
| 3-45 | 3-Cyano-N-[(1-methyl-1H-pyrazol-4-yl)methyl]-6- | LC/MS [Method 31]: |
| | (naphthalen-2-yl)-4-oxo-4,5-dihydropyrazolo[1,5-a]- | Rₜ = 1.02 min; MS |
| | pyrazine-2-carboxamide 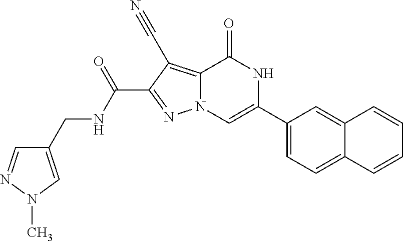 [SMILES: Cn1cc(CNC(=O)c2nn3cc(-c4ccc5ccccc5c4)[nH]c(=O)c3c2C#N)cn1] | (ESIpos): m/z = 424 [M + H]⁺. |
| | (5% of theory, 94% purity) | |
| 3-46 | 3-Cyano-6-(naphthalen-2-yl)-4-oxo-N-[(1S)-1-phenyl- | LC/MS [Method 31]: |
| | ethyl]-4,5-dihydropyrazolo[1,5-a]pyrazine-2-carbox- | Rₜ = 1.17 min; MS |
| | amide 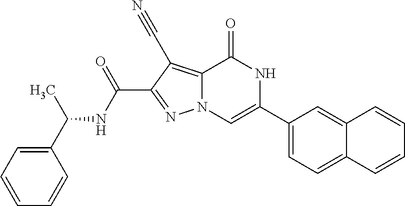 [SMILES: C[C@H](NC(=O)c1nn2cc(-c3ccc4ccccc4c3)[nH]c(=O)c2c1C#N)c1ccccc1] | (ESIpos): m/z = 434 [M + H]⁺. |
| | (4% of theory, 100% purity) | |
| 3-47 | N-[1-(3-Bromophenyl)ethyl]-3-cyano-6-(2-naphthyl)- | LC/MS [Method 31]: |
| | 4-oxo-5H-pyrazolo[1,5-a]pyrazine-2-carboxamide 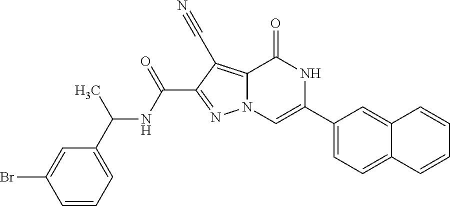 [SMILES: CC(NC(=O)c1nn2cc(-c3ccc4ccccc4c3)[nH]c(=O)c2c1C#N)c1cccc(Br)c1] | Rₜ = 1.22 min; MS (ESIpos): m/z = 512 [M + H]⁺. |
| | (20% of theory, 98% purity) | |
| 3-48 | 3-Cyano-6-(naphthalen-2-yl)-4-oxo-N-[(7R)-1,2,3,4- | LC/MS [Method 31]: |
| | tetrahydronaphthalen-1-yl]-4,5-dihydropyrazolo[1,5-a]- | Rₜ = 1.22 min; MS |
| | pyrazine-2-carboxamide 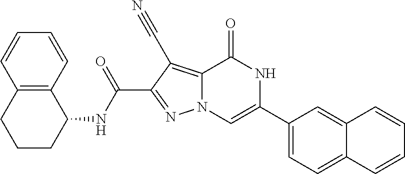 [SMILES: N#Cc1c(C(=O)N[C@@H]2CCCc3ccccc32)nn2cc(-c3ccc4ccccc4c3)[nH]c(=O)c12] | (ESIpos): m/z = 460 [M + H]⁺. |
| | (41% of theory, 97% purity) | |
| 3-49 | 3-Cyano-6-(naphthalen-2-yl)-4-oxo-N-(1-phenyl- | LC/MS [Method 31]: |
| | cyclopropyl)-4,5-dihydropyrazolo[1,5-a]pyrazine- | Rₜ = 1.17 min; MS |
| | 2-carboxamide 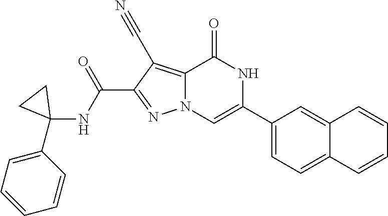 [SMILES: N#Cc1c(C(=O)NC2(c3ccccc3)CC2)nn2cc(-c3ccc4ccccc4c3)[nH]c(=O)c12] | (ESIpos): m/z = 446 [M + H]⁺. |
| | (2% of theory, 96% purity) | |
| 3-50 | 3-Cyano-N-[1-(6-methoxy-3-pyridyl)ethyl]-6-(2- | LC/MS [Method 31]: |
| | naphthyl)-4-oxo-5H-pyrazolo[1,5-a]pyrazine-2- | Rₜ = 1.11 min; MS |
| | carboxamide 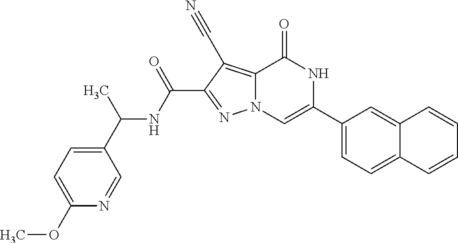 [SMILES: COc1ccc(C(C)NC(=O)c2nn3cc(-c4ccc5ccccc5c4)[nH]c(=O)c3c2C#N)cn1] | (ESIpos): m/z = 465 [M + H]⁺. |
| | (2% of theory, 100% purity) | |
| 3-51 | 3-Cyano-N-[1-(4-methoxyphenyl)ethyl]-6-(2-naphthyl)- | LC/MS [Method 31]: |
| | 4-oxo-5H-pyrazolo[1,5-a]pyrazine-2-carboxamide 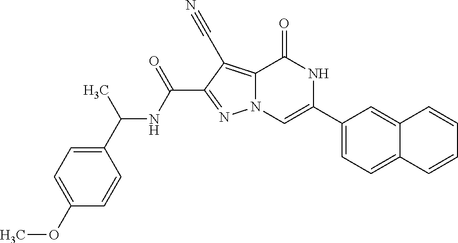 [SMILES: COc1ccc(C(C)NC(=O)c2nn3cc(-c4ccc5ccccc5c4)[nH]c(=O)c3c2C#N)cc1] | Rₜ = 1.17 min; MS (ESIpos): m/z = 464 [M + H]⁺. |
| | (2% of theory, 100% purity) | |
| 3-52 | 3-Cyano-N-[1-(4-fluorophenyl)ethyl]-6-(2-naphthyl)- | LC/MS [Method 31]: |
| | 4-oxo-5H-pyrazolo[1,5-a]pyrazine-2-carboxamide 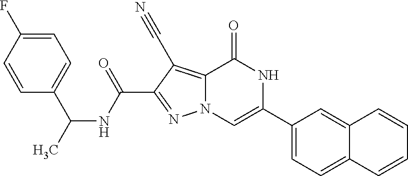 [SMILES: CC(NC(=O)c1nn2cc(-c3ccc4ccccc4c3)[nH]c(=O)c2c1C#N)c1ccc(F)cc1] | Rₜ = 1.17 min; MS (ESIpos): m/z = 452 [M + H]⁺. |
| | (17% of theory, 100% purity) | |
| 3-53 | 3-Cyano-N-[(7R)-1-(4-methylphenyl)ethyl]-6- | LC/MS [Method 31]: |
| | (naphthalen-2-yl)-4-oxo-4,5-dihydropyrazolo[1,5-a]- | Rₜ = 1.20 min; MS |
| | pyrazine-2-carboxamide 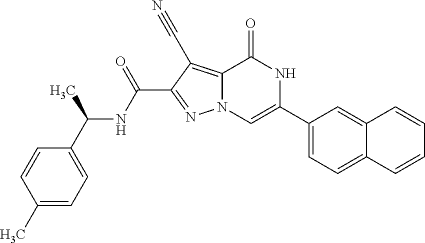 [SMILES: Cc1ccc([C@@H](C)NC(=O)c2nn3cc(-c4ccc5ccccc5c4)[nH]c(=O)c3c2C#N)cc1] | (ESIpos): m/z = 448 [M + H]⁺. |
| | (19% of theory, 100% purity) | |
| 3-54 | 3-Cyano-N-[1-(3,4-dichlorophenyl)ethyl]-6-(2- | LC/MS [Method 31]: |
| | naphthyl)-4-oxo-5H-pyrazolo[1,5-a]pyrazine-2- | Rₜ = 1.23 min; MS |
| | carboxamide 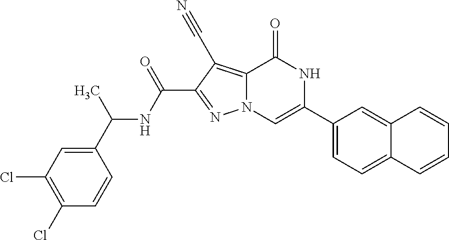 [SMILES: CC(NC(=O)c1nn2cc(-c3ccc4ccccc4c3)[nH]c(=O)c2c1C#N)c1ccc(Cl)c(Cl)c1] | (ESIpos): m/z = 502 [M + H]⁺. |
| | (12% of theory, 99% purity) | |
| 3-55 | 3-Cyano-N-(2,3-dihydro-1H-inden-2-yl)-6-(naphthalen- | LC/MS [Method 31]: |
| | 2-yl)-4-oxo-4,5-dihydropyrazolo[1,5-a]pyrazine- | Rₜ = 1.20 min; MS |
| | 2-carboxamide 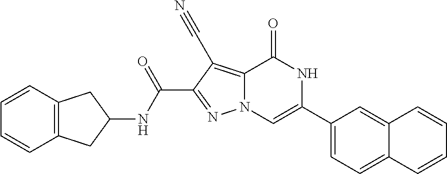 [SMILES: N#Cc1c(C(=O)NC2Cc3ccccc3C2)nn2cc(-c3ccc4ccccc4c3)[nH]c(=O)c12] | (ESIpos): m/z = 446 [M + H]⁺. |
| | (6% of theory, 95% purity) | |
| 3-56 | 3-Cyano-N-[cyclopropyl(4-methoxyphenyl)methyl]- | LC/MS [Method 31]: |
| | 6-(2-naphthyl)-4-oxo-5H-pyrazolo[1,5-a]pyrazine- | Rₜ = 1.20 min; MS |
| | 2-carboxamide 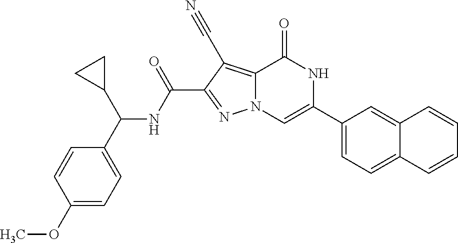 [SMILES: COc1ccc(C(NC(=O)c2nn3cc(-c4ccc5ccccc5c4)[nH]c(=O)c3c2C#N)C2CC2)cc1] | (ESIpos): m/z = 490 [M + H]⁺. |
| | (8% of theory, 90% purity) | |
| 3-57 | N-[1-(4-Methoxyphenyl)ethyl]-3,7-dimethyl-6-(2- | LC/MS [Method 31]: |
| | naphthyl)-4-oxo-5H-pyrazolo[1,5-a]pyrazine- | Rₜ = 1.24 min; MS |
| | 2-carboxamide 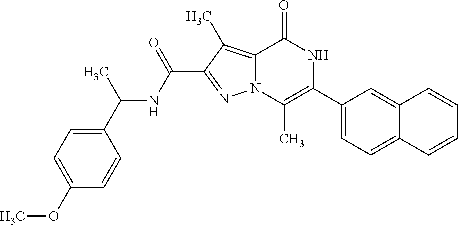 [SMILES: COc1ccc(C(C)NC(=O)c2nn3c(C)c(-c4ccc5ccccc5c4)[nH]c(=O)c3c2C)cc1] | (ESIpos): m/z = 467 [M + H]⁺. |
| | (2% of theory, 100% purity) | |
| 3-58 | 3-Cyano-N-[1-(2,4-dichlorophenyl)ethyl]-6-(2-naphthyl)- | LC/MS [Method 31]: |
| | 4-oxo-5H-pyrazolo[1,5-a]pyrazine-2-carboxamide 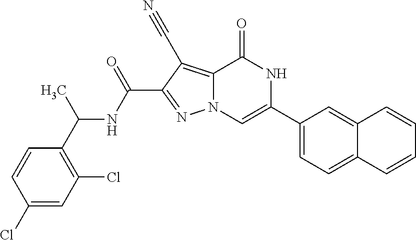 [SMILES: CC(NC(=O)c1nn2cc(-c3ccc4ccccc4c3)[nH]c(=O)c2c1C#N)c1ccc(Cl)cc1Cl] | Rₜ = 1.24 min; MS (ESIpos): m/z = 502 [M + H]⁺. |
| | (5% of theory, 96% purity) | |
| 3-59 | 3-Cyano-N-[1-(4-fluorophenyl)-2-methylpropyl]-6-(2- | LC/MS [Method 31]: |
| | naphthyl)-4-oxo-5H-pyrazolo[1,5-a]pyrazine- | Rₜ = 1.23 min; MS |
| | 2-carboxamide 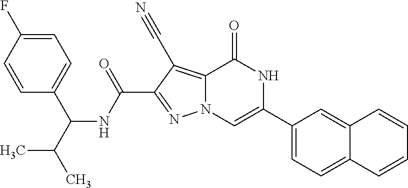 [SMILES: CC(C)C(NC(=O)c1nn2cc(-c3ccc4ccccc4c3)[nH]c(=O)c2c1C#N)c1ccc(F)cc1] | (ESIpos): m/z = 480 [M + H]⁺. |
| | (19% of theory, 100% purity) | |
| 3-60 | 3-Cyano-N-[1-[2-(difluoromethoxy)phenyl]ethyl]-6- | LC/MS [Method 31]: |
| | (2-naphthyl)-4-oxo-5H-pyrazolo[1,5-a]pyrazine- | Rₜ = 1.21 min; MS |
| | 2-carboxamide 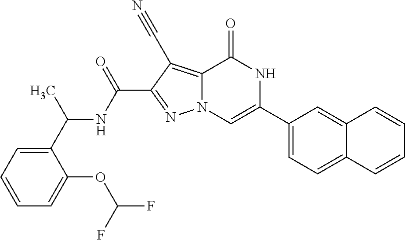 [SMILES: CC(NC(=O)c1nn2cc(-c3ccc4ccccc4c3)[nH]c(=O)c2c1C#N)c1ccccc1OC(F)F] | (ESIpos): m/z = 500 [M + H]⁺. |
| | (23% of theory, 95% purity) | |
| 3-61 | 3-Cyano-6-(naphthalen-2-yl)-4-oxo-N-[(1R)-1-phenyl- | LC/MS [Method 31]: |
| | propyl]-4,5-dihydropyrazolo[1,5-a]pyrazine- | Rₜ = 1.20 min; MS |
| | 2-carboxamide 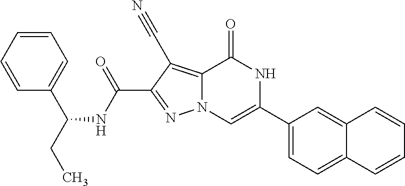 [SMILES: CC[C@@H](NC(=O)c1nn2cc(-c3ccc4ccccc4c3)[nH]c(=O)c2c1C#N)c1ccccc1] | (ESIpos): m/z = 448 [M + H]⁺. |
| | (16% of theory, 100% purity) | |
| 3-62 | N-[1-(4-Chlorophenyl)cyclobutyl]-3-cyano-6- | LC/MS [Method 31]: |
| | (naphthalen-2-yl)-4-oxo-4,5-dihydropyrazolo[1,5-a]- | Rₜ = 1.24 min; MS |
| | pyrazine-2-carboxamide 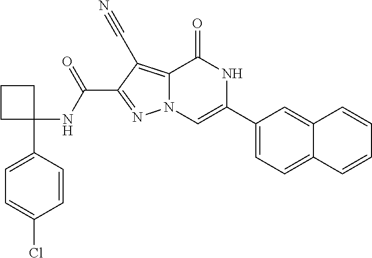 [SMILES: N#Cc1c(C(=O)NC2(c3ccc(Cl)cc3)CCC2)nn2cc(-c3ccc4ccccc4c3)[nH]c(=O)c12] | (ESIpos): m/z = 494 [M + H]⁺. |
| | (18% of theory, 95% purity) | |
| 3-63 | 3-Cyano-N-[(1S)-3-hydroxy-1-(4- | LC/MS [Method 31]: |
| | methoxyphenyl)propyl]-6-(naphthalen-2-yl)-4-oxo-4,5- | Rₜ = 1.06 min; MS |
| | dihydropyrazolo[1,5-a]pyrazine-2-carboxamide 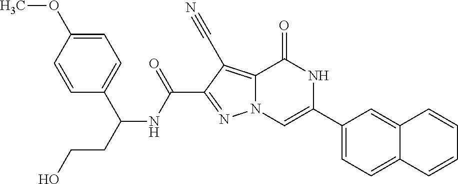 [SMILES: COc1ccc(C(CCO)NC(=O)c2nn3cc(-c4ccc5ccccc5c4)[nH]c(=O)c3c2C#N)cc1] | (ESIpos): m/z = 494 [M + H]⁺. |
| | (17% of theory, 90% purity) | |
| 3-64 | 3,7-Dimethyl-6-(naphthalen-2-yl)-4-oxo-N-[(pyridin-4- | LC/MS [Method 31]: |
| | yl)methyl]-4,5-dihydropyrazolo[1,5-a]pyrazine- | Rₜ = 0.88 min; MS |
| | 2-carboxamide 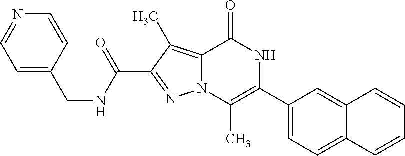 [SMILES: Cc1c(C(=O)NCc2ccncc2)nn2c(C)c(-c3ccc4ccccc4c3)[nH]c(=O)c12] | (ESIpos): m/z = 424 [M + H]⁺. |
| | (4% of theory, 100% purity) | |
| 3-65 | 3,7-Dimethyl-6-(naphthalen-2-yl)-4-oxo-N-[(1R)-1- | LC/MS [Method 31]: |
| | phenylethyl]-4,5-dihydropyrazolo[1,5-a]pyrazine-2- | Rₜ = 1.24 min; MS |
| | carboxamide 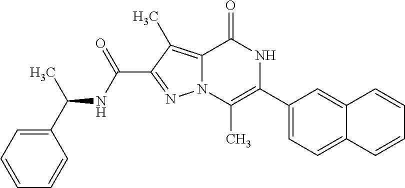 [SMILES: Cc1c(C(=O)N[C@H](C)c2ccccc2)nn2c(C)c(-c3ccc4ccccc4c3)[nH]c(=O)c12] | (ESIpos): m/z = 437 [M + H]⁺. |
| | (10% of theory, 100% purity) | |
| 3-66 | 3,7-Dimethyl-6-(naphthalen-2-yl)-4-oxo-N-[(7S)-1- | LC/MS [Method 31]: |
| | (pyrazin-2-yl)ethyl]-4,5-dihydropyrazolo[1,5-a]pyrazine- | Rₜ = 1.16 min; MS |
| | 2-carboxamide 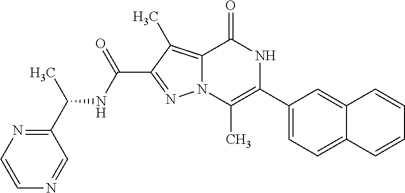 [SMILES: Cc1c(C(=O)N[C@@H](C)c2cnccn2)nn2c(C)c(-c3ccc4ccccc4c3)[nH]c(=O)c12] | (ESIpos): m/z = 439 [M + H]⁺. |
| | (25% of theory, 93% purity) | |
| 3-67 | N-[(7S,2R)-2-Hydroxy-2,3-dihydro-1H-inden-1-yl]-3,7- | LC/MS [Method 31]: |
| | dimethyl-6-(naphthalen-2-yl)-4-oxo-4,5-dihydropyr- | Rₜ = 1.16 min; MS |
| | azolo[1,5-a]pyrazine-2-carboxamide 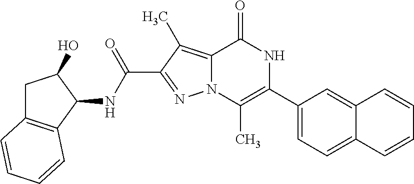 [SMILES: Cc1c(C(=O)N[C@H]2c3ccccc3C[C@H]2O)nn2c(C)c(-c3ccc4ccccc4c3)[nH]c(=O)c12] | (ESIpos): m/z = 465 [M + H]⁺. |
| | (23% of theory, 100% purity) | |
| 3-68 | N-[1-(2-Chlorophenyl)-3-hydroxypropyl]-3,7-dimethyl- | LC/MS [Method 31]: |
| | 6-(2-naphthyl)-4-oxo-5H-pyrazolo[1,5-a]pyrazine- | Rₜ = 1.18 min; MS |
| | 2-carboxamide 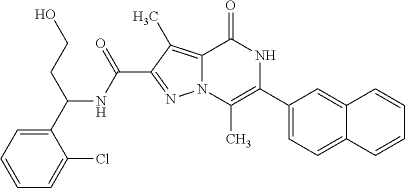 [SMILES: Cc1c(C(=O)NC(CCO)c2ccccc2Cl)nn2c(C)c(-c3ccc4ccccc4c3)[nH]c(=O)c12] | (ESIpos): m/z = 501 [M + H]⁺. |
| | (24% of theory, 96% purity) | |
| 3-69 | N-[1-(4-Bromophenyl)-2-hydroxyethyl]-3,7-dimethyl- | LC/MS [Method 31]: |
| | 6-(2-naphthyl)-4-oxo-5H-pyrazolo[1,5-a]pyrazine- | Rₜ = 1.16 min; MS |
| | 2-carboxamide 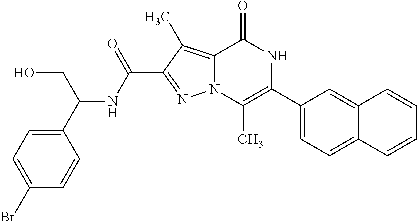 [SMILES: Cc1c(C(=O)NC(CO)c2ccc(Br)cc2)nn2c(C)c(-c3ccc4ccccc4c3)[nH]c(=O)c12] | (ESIpos): m/z = 531 [M + H]⁺. |
| | (26% of theory, 100% purity) | |
| 3-70 | 3,7-Dimethyl-6-(naphthalen-2-yl)-4-oxo-N-[(pyridin-2- | LC/MS [Method 31]: |
| | yl)methyl]-4,5-dihydropyrazolo[1,5-a]pyrazine- | Rₜ = 1.01 min; MS |
| | 2-carboxamide 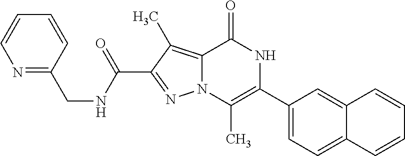 [SMILES: Cc1c(C(=O)NCc2ccccn2)nn2c(C)c(-c3ccc4ccccc4c3)[nH]c(=O)c12] | (ESIpos): m/z = 424 [M + H]⁺. |
| | (8% of theory, 100% purity) | |
| 3-71 | N-[(1R)-2-Hydroxy-1-phenylethyl]-3,7-dimethyl-6- | LC/MS [Method 31]: |
| | (naphthalen-2-yl)-4-oxo-4,5-dihydropyrazolo[1,5-a]- | Rₜ = 1.12 min; MS |
| | pyrazine-2-carboxamide 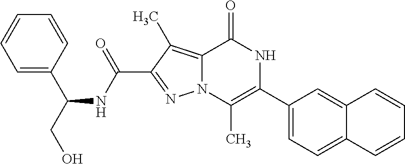 [SMILES: Cc1c(C(=O)N[C@@H](CO)c2ccccc2)nn2c(C)c(-c3ccc4ccccc4c3)[nH]c(=O)c12] | (ESIpos): m/z = 453 [M + H]⁺. |
| | (31% of theory, 100% purity) | |
| 3-72 | N-[2,2-Difluoro-1-(3-fluorophenyl)ethyl]-3,7-dimethyl- | LC/MS [Method 31]: |
| | 6-(2-naphthyl)-4-oxo-5H-pyrazolo[1,5-a]pyrazine- | Rₜ = 1.25 min; MS |
| | 2-carboxamide 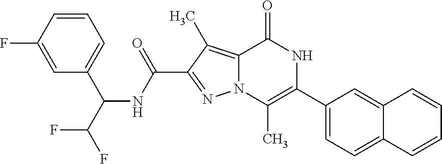 [SMILES: Cc1c(C(=O)NC(c2cccc(F)c2)C(F)F)nn2c(C)c(-c3ccc4ccccc4c3)[nH]c(=O)c12] | (ESIpos): m/z = 491 [M + H]⁺. |
| | (32% of theory, 98% purity) | |
| 3-73 | 3,7-Dimethyl-6-(2-naphthyl)-4-oxo-N-[1-(3-pyridyl)- | LC/MS [Method 31]: |
| | ethyl]-5H-pyrazolo[1,5-a]pyrazine-2-carboxamide 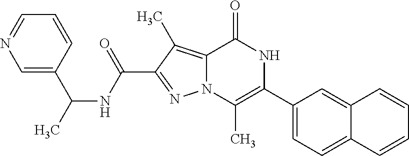 [SMILES: Cc1c(C(=O)NC(C)c2cccnc2)nn2c(C)c(-c3ccc4ccccc4c3)[nH]c(=O)c12] | Rₜ = 0.97 min; MS (ESIpos): m/z = 438 [M + H]⁺. |
| | (10% of theory, 100% purity) | |
| 3-74 | N-[1-(3-Fluoro-4-methoxyphenyl)ethyl]-3,7-dimethyl- | LC/MS [Method 31]: |
| | 6-(2-naphthyl)-4-oxo-5H-pyrazolo[1,5-a]pyrazine- | Rₜ = 1.24 min; MS |
| | 2-carboxamide 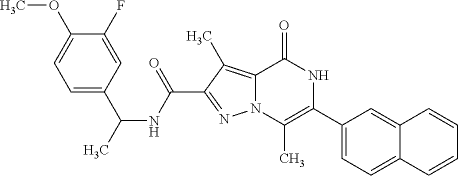 [SMILES: COc1ccc(C(C)NC(=O)c2nn3c(C)c(-c4ccc5ccccc5c4)[nH]c(=O)c3c2C)cc1F] | (ESIpos): m/z = 485 [M + H]⁺. |
| | (24% of theory, 100% purity) | |
| 3-75 | N-(2,3-Dihydro-1H-inden-1-yl)-3,7-dimethyl-6-(2- | LC/MS [Method 31]: |
| | naphthyl)-4-oxo-4,5-dihydropyrazolo[1,5-a]pyrazine- | Rₜ = 1.27 min; MS |
| | 2-carboxamide 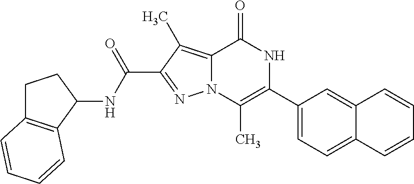 [SMILES: Cc1c(C(=O)NC2CCc3ccccc32)nn2c(C)c(-c3ccc4ccccc4c3)[nH]c(=O)c12] | (ESIpos): m/z = 449 [M + H]⁺. |
| | (7% of theory, 94% purity) | |
| 3-76 | 3-Cyano-N-[1-(3,5-dichlorophenyl)-2-hydroxyethyl]-6- | LC/MS [Method 31]: |
| | (2-naphthyl)-4-oxo-5H-pyrazolo[1,5-a]pyrazine- | Rₜ = 1.15 min; MS |
| | 2-carboxamide 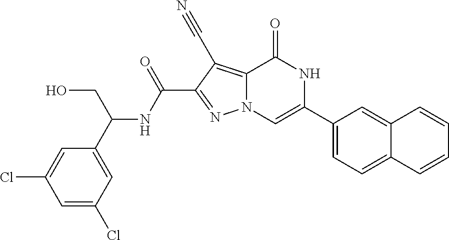 [SMILES: N#Cc1c(C(=O)NC(CO)c2cc(Cl)cc(Cl)c2)nn2cc(-c3ccc4ccccc4c3)[nH]c(=O)c12] | (ESIpos): m/z = 518 [M + H]⁺. |
| | (12% of theory, 75% purity) | |
| 3-77 | 3-Cyano-N-[2-(3-cyclopropyl-1H-pyrazol-1-yl)ethyl]-6- | LC/MS [Method 31]: |
| | (naphthalen-2-yl)-4-oxo-4,5-dihydropyrazolo[1,5-a]- | Rₜ = 1.10 min; MS |
| | pyrazine-2-carboxamide 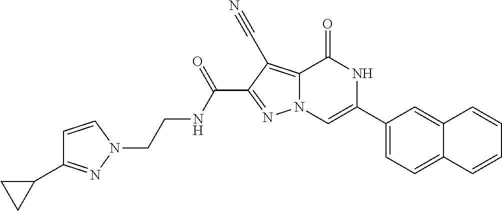 [SMILES: N#Cc1c(C(=O)NCCn2ccc(C3CC3)n2)nn2cc(-c3ccc4ccccc4c3)[nH]c(=O)c12] | (ESIpos): m/z = 464 [M + H]⁺. |
| | (7% of theory, 85% purity) | |

General Procedure 4 (Parallel Synthesis of Example Compounds):

The corresponding amine was placed on a multi-well plate, and a solution of 3-isopropyl-6-(5-methylquinolin-3-yl)-4-oxo-4,5-dihydropyrazolo[1,5-a]pyrazine-2-carboxylic acid (Intermediate 77A, 26.2 mg, 100 μmol) in DMF (400 pl) was added, followed by a mixture of 1-(3-dimethylaminopropyl)-3-ethylcarbodiimide hydrochloride (28.8 mg, 150 μmol) and 1-hydroxybenzotriazole (23.0 mg, 150 μmol) in DMF (400 pl). Then, N-methylmorpholine (50 μl, 393 μmol) was added, and the plate was shaken at RT for three days. After this, the reaction mixture was filtered, and the filtrate solution was directly purified by preparative HPLC (Method P11). The fractions containing the desired product were combined, evaporated and dried in vacuo.

The example compounds specified in the following Table 3 were prepared according to general procedure 4:

TABLE 3

| | IUPAC name/structure | |
|---|---|---|
| Example | (yield, purity) | Analytical data |
| 4-1 | N-[2-(Dimethylamino)-1-phenylethyl]-3-isopropyl-6- | LC/MS [Method 31]: |
| | (5-methyl-3-quinolyl)-4-oxo-5H-pyrazolo[1,5-a]- | Rₜ = 0.85 min; MS |
| | pyrazine-2-carboxamide 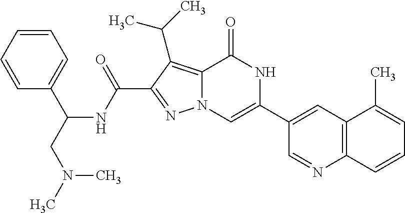 [SMILES: Cc1cccc2ncc(-c3cn4nc(C(=O)NC(CN(C)C)c5ccccc5)c(C(C)C)c4c(=O)[nH]3)cc12] | (ESIpos): m/z = 509 [M + H]⁺. |
| | (55% of theory, 97% purity) | |
| 4-2 | N-({3-[(Dimethylamino)methyl]phenyl}methyl)-6- | LC/MS [Method 31]: |
| | (5-methylquinolin-3-yl)-4-oxo-3-(propan-2-yl)-4,5- | Rₜ = 0.81 min; MS |
| | dihydropyrazolo[1,5-a]pyrazine-2-carboxamide 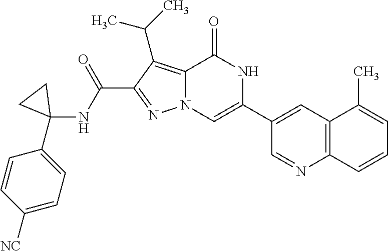 [SMILES: Cc1cccc2ncc(-c3cn4nc(C(=O)NC5(c6ccc(C#N)cc6)CC5)c(C(C)C)c4c(=O)[nH]3)cc12] | (ESIpos): m/z = 509 [M + H]⁺. |
| | (19% of theory, 98% purity) | |
| 4-3 | N-[1-(4-Cyanophenyl)cyclopropyl]-6-(5-methylquinolin- | LC/MS [Method 31]: |
| | 3-yl)-4-oxo-3-(propan-2-yl)-4,5-dihydropyrazolo[1,5-a]- | Rₜ = 1.20 min; MS |
| | pyrazine-2-carboxamide 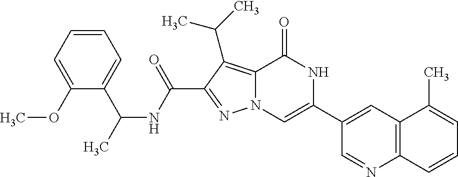 | (ESIpos): m/z = 503 [M + H]⁺. |
| | (26% of theory, 92% purity) | |
| 4-4 | 3-Isopropyl-N-[1-(2-methoxyphenyl)ethyl]-6-(5-methyl- | LC/MS [Method 31]: |
| | 3-quinolyl)-4-oxo-5H-pyrazolo[1,5-a]pyrazine- | Rₜ = 1.26 min; MS |
| | 2-carboxamide 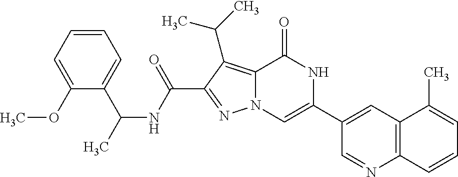 [SMILES: COc1ccccc1C(C)NC(=O)c1nn2cc(-c3cnc4cccc(C)c4c3)[nH]c(=O)c2c1C(C)C] | (ESIpos): m/z = 496 [M + H]⁺. |
| | (40% of theory, 100% purity) | |
| 4-5 | 6-(5-Methylquinolin-3-yl)-4-oxo-3-(propan-2-yl)-N- | LC/MS [Method 31]: |
| | [(7R)-1,2,3,4-tetrahydronaphthalen-1-yl]-4,5-dihydro- | Rₜ = 1.28 min; MS |
| | pyrazolo[1,5-a]pyrazine-2-carboxamide 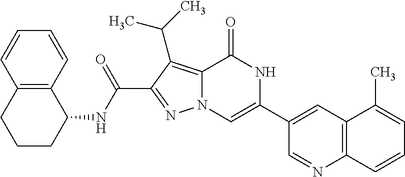 [SMILES: Cc1cccc2ncc(-c3cn4nc(C(=O)N[C@@H]5CCCc6ccccc65)c(C(C)C)c4c(=O)[nH]3)cc12] | (ESIpos): m/z = 492 [M + H]⁺. |
| | (36% of theory, 100% purity) | |
| 4-6 | 3-Isopropyl-N-[1-(6-methoxy-3-pyridyl)ethyl]-6- | LC/MS [Method 31]: |
| | (5-methyl-3-quinolyl)-4-oxo-5H-pyrazolo[1,5-a]- | Rₜ = 1.17 min; MS |
| | pyrazine-2-carboxamide 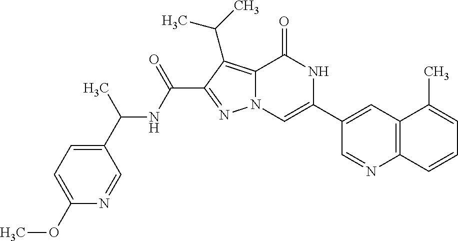 [SMILES: COc1ccc(C(C)NC(=O)c2nn3cc(-c4cnc5cccc(C)c5c4)[nH]c(=O)c3c2C(C)C)cn1] | (ESIpos): m/z = 497 [M + H]⁺. |
| | (42% of theory, 100% purity) | |
| 4-7 | 3-Isopropyl-N-[1-(4-methoxyphenyl)ethyl]-6-(5-methyl- | LC/MS [Method 31]: |
| | 3-quinolyl)-4-oxo-5H-pyrazolo[1,5-a]pyrazine- | Rₜ = 1.23 min; MS |
| | 2-carboxamide 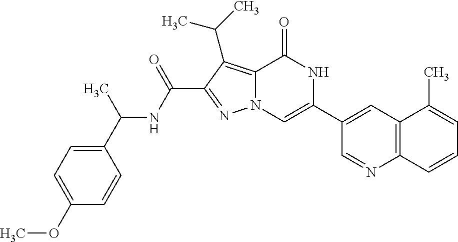 [SMILES: COc1ccc(C(C)NC(=O)c2nn3cc(-c4cnc5cccc(C)c5c4)[nH]c(=O)c3c2C(C)C)cc1] | (ESIpos): m/z = 496 [M + H]⁺. |
| | (43% of theory, 100% purity) | |
| 4-8 | N-[2-(3,5-Dichlorophenyl)propan-2-yl]-6-(5-methyl- | LC/MS [Method 31]: |
| | quinolin-3-yl)-4-oxo-3-(propan-2-yl)-4,5-dihydropyr- | Rₜ = 1.32 min; MS |
| | azolo[1,5-a]pyrazine-2-carboxamide 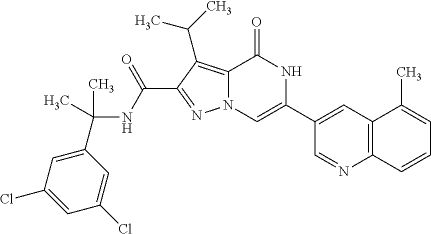 [SMILES: Cc1cccc2ncc(-c3cn4nc(C(=O)NC(C)(C)c5cc(Cl)cc(Cl)c5)c(C(C)C)c4c(=O)[nH]3)cc12] | (ESIpos): m/z = 548 [M + H]⁺. |
| | (39% of theory, 100% purity) | |
| 4-9 | N-[1-(4-Ethylphenyl)ethyl]-3-isopropyl-6-(5-methyl- | LC/MS [Method 31]: |
| | 3-quinolyl)-4-oxo-5H-pyrazolo[1,5-a]pyrazine- | Rₜ = 1.29 min; MS |
| | 2-carboxamide 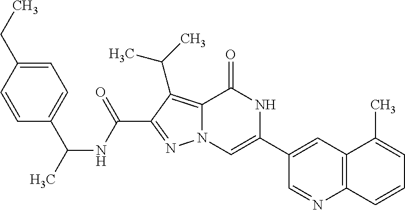 [SMILES: CCc1ccc(C(C)NC(=O)c2nn3cc(-c4cnc5cccc(C)c5c4)[nH]c(=O)c3c2C(C)C)cc1] | (ESIpos): m/z = 494 [M + H]⁺. |
| | (45% of theory, 100% purity) | |
| 4-10 | N-[(2,3-Dimethyl-1H-indol-5-yl)methyl]-6-(5-methyl- | LC/MS [Method 31]: |
| | quinolin-3-yl)-4-oxo-3-(propan-2-yl)-4,5-dihydropyr- | Rₜ = 1.23 min; MS |
| | azolo[1,5-a]pyrazine-2-carboxamide 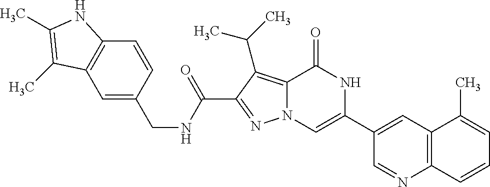 [SMILES: Cc1[nH]c2ccc(CNC(=O)c3nn4cc(-c5cnc6cccc(C)c6c5)[nH]c(=O)c4c3C(C)C)cc2c1C] | (ESIpos): m/z = 519 [M + H]⁺. |
| | (56% of theory, 100% purity) | |
| 4-11 | N-[1-(4-Fluorophenyl)ethyl]-3-isopropyl-6-(5-methyl- | LC/MS [Method 31]: |
| | 3-quinolyl)-4-oxo-5H-pyrazolo[1,5-a]pyrazine- | Rₜ = 1.23 min; MS |
| | 2-carboxamide 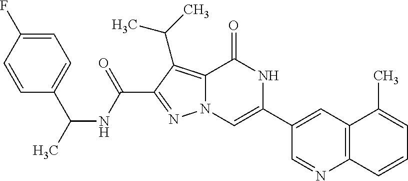 [SMILES: Cc1cccc2ncc(-c3cn4nc(C(=O)NC(C)c5ccc(F)cc5)c(C(C)C)c4c(=O)[nH]3)cc12] | (ESIpos): m/z = 484 [M + H]⁺. |
| | (49% of theory, 100% purity) | |
| 4-12 | N-(1,1-Dioxothiolan-3-yl)-3-isopropyl-6-(5-methyl- | LC/MS [Method 31]: |
| | 3-quinolyl)-4-oxo-5H-pyrazolo[1,5-a]pyrazine- | Rₜ = 1.03 min; MS |
| | 2-carboxamide 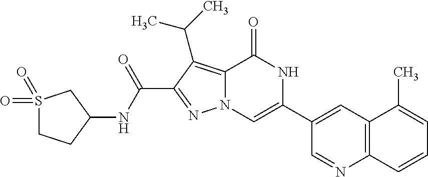 [SMILES: Cc1cccc2ncc(-c3cn4nc(C(=O)NC5CCS(=O)(=O)C5)c(C(C)C)c4c(=O)[nH]3)cc12] | (ESIpos): m/z = 480 [M + H]⁺. |
| | (60% of theory, 100% purity) | |
| 4-13 | N-[1-(1-Ethylpyrazol-3-yl)ethyl]-3-isopropyl-6-(5- | LC/MS [Method 31]: |
| | methyl-3-quinolyl)-4-oxo-5H-pyrazolo[1,5-a]pyrazine- | Rₜ = 1.14 min; MS |
| | 2-carboxamide 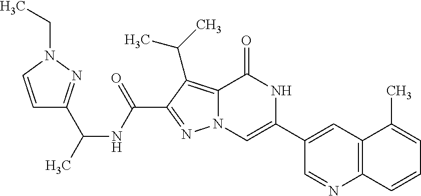 [SMILES: CCn1ccc(C(C)NC(=O)c2nn3cc(-c4cnc5cccc(C)c5c4)[nH]c(=O)c3c2C(C)C)n1] | (ESIpos): m/z = 484 [M + H]⁺. |
| | (57% of theory, 100% purity) | |
| 4-14 | 6-(5-Methylquinolin-3-yl)-4-oxo-3-(propan-2-yl)-N-[(1H- | LC/MS [Method 31]: |
| | pyrazol-3-yl)methyl]-4,5-dihydropyrazolo[1,5-a]- | Rₜ = 1.02 min; MS |
| | pyrazine-2-carboxamide 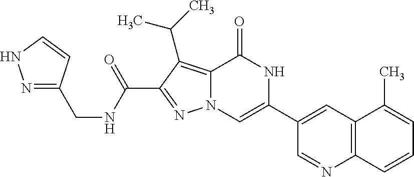 [SMILES: Cc1cccc2ncc(-c3cn4nc(C(=O)NCc5cc[nH]n5)c(C(C)C)c4c(=O)[nH]3)cc12] | (ESIpos): m/z = 442 [M + H]⁺. |
| | (38% of theory, 100% purity) | |
| 4-15 | 6-(5-Methylquinolin-3-yl)-4-oxo-3-(propan-2-yl)-N- | LC/MS [Method 31]: |
| | [1-(propan-2-yl)-1H-pyrazol-5-yl]-4,5-dihydropyrazolo- | Rₜ = 1.15 min; MS |
| | [1,5-a]pyrazine-2-carboxamide 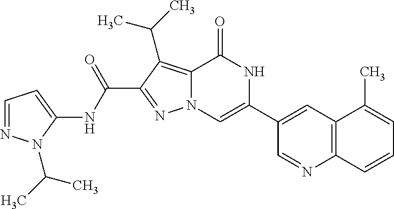 [SMILES: Cc1cccc2ncc(-c3cn4nc(C(=O)Nc5ccnn5C(C)C)c(C(C)C)c4c(=O)[nH]3)cc12] | (ESIpos): m/z = 470 [M + H]⁺. |
| | (53% of theory, 100% purity) | |
| 4-16 | N-[(1-Ethyl-1H-pyrazol-5-yl)methyl]-6-(5-methyl- | LC/MS [Method 31]: |
| | quinolin-3-yl)-4-oxo-3-(propan-2-yl)-4,5-dihydropyr- | Rₜ = 1.13 min; MS |
| | azolo[1,5-a]pyrazine-2-carboxamide 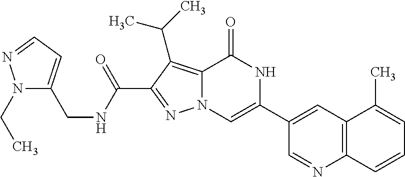 [SMILES: CCn1nccc1CNC(=O)c1nn2cc(-c3cnc4cccc(C)c4c3)[nH]c(=O)c2c1C(C)C] | (ESIpos): m/z = 470 [M + H]⁺. |
| | (55% of theory, 100% purity) | |
| 4-17 | 3-Isopropyl-N-(3-methyl-1,1-dioxothiolan-3-yl)-6- | LC/MS [Method 31]: |
| | (5-methyl-3-quinolyl)-4-oxo-5H-pyrazolo[1,5-a]- | Rₜ = 1.07 min; MS |
| | pyrazine-2-carboxamide 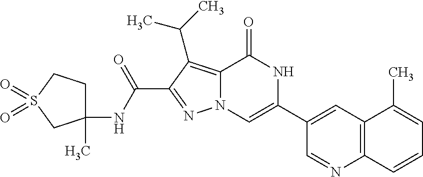 [SMILES: Cc1cccc2ncc(-c3cn4nc(C(=O)NC5(C)CCS(=O)(=O)C5)c(C(C)C)c4c(=O)[nH]3)cc12] | (ESIpos): m/z = 494 [M + H]⁺. |
| | (62% of theory, 100% purity) | |
| 4-18 | N-(1-Cyclopropylpropan-2-yl)-3-isopropyl-6-(5-methyl- | LC/MS [Method 31]: |
| | quinolin-3-yl)-4-oxo-4,5-dihydropyrazolo[1,5-a]- | Rₜ = 1.23 min; MS |
| | pyrazine-2-carboxamide 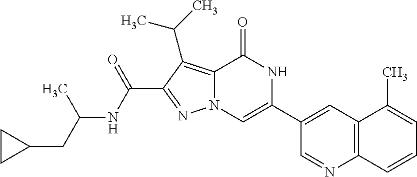 [SMILES: Cc1cccc2ncc(-c3cn4nc(C(=O)NC(C)CC5CC5)c(C(C)C)c4c(=O)[nH]3)cc12] | (ESIpos): m/z = 444 [M + H]⁺. |
| | (56% of theory, 100% purity) | |
| 4-19 | N-(1-Methyl-1H-pyrazol-5-yl)-6-(5-methylquinolin-3-yl)- | LC/MS [Method 31]: |
| | 4-oxo-3-(propan-2-yl)-4,5-dihydropyrazolo[1,5-a]- | Rₜ = 1.12 min; MS |
| | pyrazine-2-carboxamide 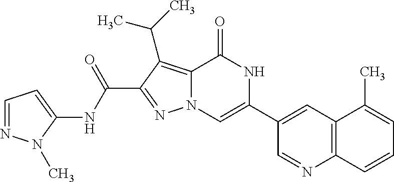 [SMILES: Cc1cccc2ncc(-c3cn4nc(C(=O)Nc5ccnn5C)c(C(C)C)c4c(=O)[nH]3)cc12] | (ESIpos): m/z = 442 [M + H]⁺. |
| | (15% of theory, 100% purity) | |
| 4-20 | N-[(4-Ethyl-4H-1,2,4-triazol-3-yl)methyl]-6-(5-methyl- | LC/MS [Method 31]: |
| | quinolin-3-yl)-4-oxo-3-(propan-2-yl)-4,5-dihydropyr- | Rₜ = 1.07 min; MS |
| | azolo[1,5-a]pyrazine-2-carboxamide 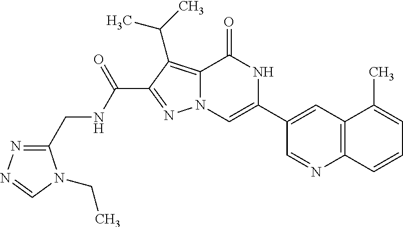 [SMILES: CCn1cnnc1CNC(=O)c1nn2cc(-c3cnc4cccc(C)c4c3)[nH]c(=O)c2c1C(C)C] | (ESIpos): m/z = 471 [M + H]⁺. |
| | (32% of theory, 100% purity) | |
| 4-21 | 3-Isopropyl-6-(5-methyl-3-quinolyl)-4-oxo-N- | LC/MS [Method 31]: |
| | (tetrahydrofuran-3-yl)-5H-pyrazolo[1,5-a]pyrazine-2- | Rₜ = 1.07 min; MS |
| | carboxamide 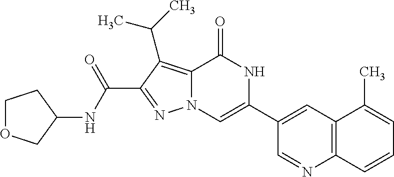 [SMILES: Cc1cccc2ncc(-c3cn4nc(C(=O)NC5CCOC5)c(C(C)C)c4c(=O)[nH]3)cc12] | (ESIpos): m/z = 432 [M + H]⁺. |
| | (39% of theory, 100% purity) | |
| 4-22 | N-[(1-Methyl-1H-pyrazol-5-yl)methyl]-6-(5-methyl- | LC/MS [Method 31]: |
| | quinolin-3-yl)-4-oxo-3-(propan-2-yl)-4,5-dihydropyr- | Rₜ = 1.11 min; MS |
| | azolo[1,5-a]pyrazine-2-carboxamide 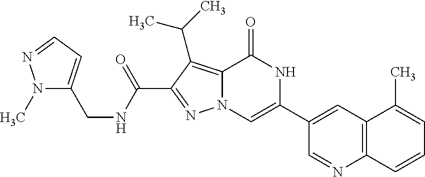 [SMILES: Cc1cccc2ncc(-c3cn4nc(C(=O)NCc5ccnn5C)c(C(C)C)c4c(=O)[nH]3)cc12] | (ESIpos): m/z = 456 [M + H]⁺. |
| | (29% of theory, 100% purity) | |
| 4-23 | N-[(4-Chloro-1-ethyl-1H-pyrazol-3-yl)methyl]-6- | LC/MS [Method 31]: |
| | (5-methylquinolin-3-yl)-4-oxo-3-(propan-2-yl)-4,5- | Rₜ = 1.19 min; MS |
| | dihydropyrazolo[1,5-a]pyrazine-2-carboxamide 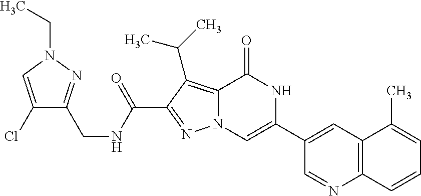 [SMILES: CCn1cc(Cl)c(CNC(=O)c2nn3cc(-c4cnc5cccc(C)c5c4)[nH]c(=O)c3c2C(C)C)n1] | (ESIpos): m/z = 504 [M + H]⁺. |
| | (44% of theory, 97% purity) | |
| 4-24 | N-(1-Methyl-1H-pyrazol-4-yl)-6-(5-methylquinolin-3-yl)- | LC/MS [Method 31]: |
| | 4-oxo-3-(propan-2-yl)-4,5-dihydropyrazolo[1,5-a]- | Rₜ = 1.12 min; MS |
| | pyrazine-2-carboxamide 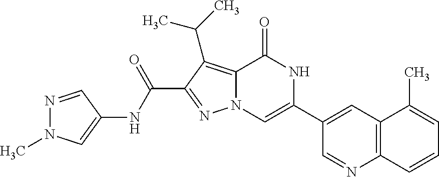 [SMILES: Cc1cccc2ncc(-c3cn4nc(C(=O)Nc5cnn(C)c5)c(C(C)C)c4c(=O)[nH]3)cc12] | (ESIpos): m/z = 442 [M + H]⁺. |
| | (56% of theory, 100% purity) | |
| 4-25 | 6-(5-Methylquinolin-3-yl)-4-oxo-3-(propan-2-yl)-N- | LC/MS [Method 31]: |
| | [(pyridin-4-yl)methyl]-4,5-dihydropyrazolo[1,5-a]- | Rₜ = 0.87 min; MS |
| | pyrazine-2-carboxamide 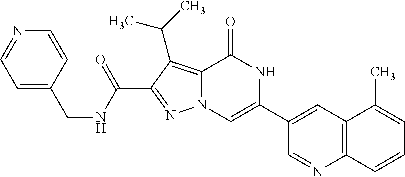 [SMILES: Cc1cccc2ncc(-c3cn4nc(C(=O)NCc5ccncc5)c(C(C)C)c4c(=O)[nH]3)cc12] | (ESIpos): m/z = 453 [M + H]⁺. |
| | (15% of theory, 100% purity) | |
| 4-26 | 6-(5-Methylquinolin-3-yl)-4-oxo-3-(propan-2-yl)-N- | LC/MS [Method 31]: |
| | [(pyridin-2-yl)methyl]-4,5-dihydropyrazolo[1,5-a]- | Rₜ = 1.02 min; MS |
| | pyrazine-2-carboxamide 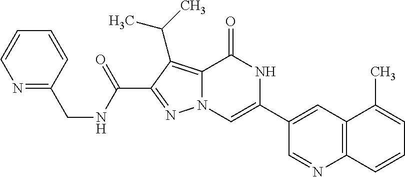 [SMILES: Cc1cccc2ncc(-c3cn4nc(C(=O)NCc5ccccn5)c(C(C)C)c4c(=O)[nH]3)cc12] | (ESIpos): m/z = 453 [M + H]⁺. |
| | (32% of theory, 100% purity) | |
| 4-27 | 6-(5-Methylquinolin-3-yl)-4-oxo-N-[(1R)-1-phenylethyl]- | LC/MS [Method 31]: |
| | 3-(propan-2-yl)-4,5-dihydropyrazolo[1,5-a]pyrazine- | Rₜ = 1.23 min; MS |
| | 2-carboxamide 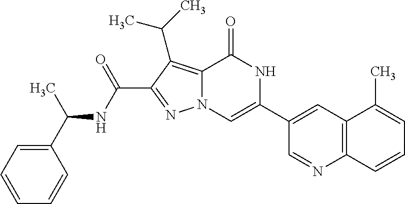 [SMILES: Cc1cccc2ncc(-c3cn4nc(C(=O)N[C@H](C)c5ccccc5)c(C(C)C)c4c(=O)[nH]3)cc12] | (ESIpos): m/z = 466 [M + H]⁺. |
| | (36% of theory, 100% purity) | |
| 4-28 | 6-(5-Methylquinolin-3-yl)-4-oxo-3-(propan-2-yl)-N- | LC/MS [Method 31]: |
| | [(7S)-1-(pyrazin-2-yl)ethyl]-4,5-dihydropyrazolo[1,5-a]- | Rₜ = 1.14 min; MS |
| | pyrazine-2-carboxamide 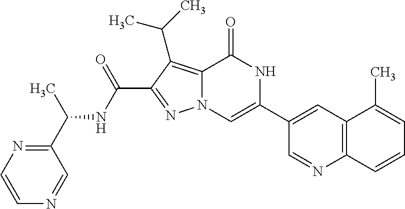 [SMILES: Cc1cccc2ncc(-c3cn4nc(C(=O)N[C@@H](C)c5cnccn5)c(C(C)C)c4c(=O)[nH]3)cc12] | (ESIpos): m/z = 468 [M + H]⁺. |
| | (51% of theory, 100% purity) | |
| 4-29 | 6-(5-Methylquinolin-3-yl)-4-oxo-3-(propan-2-yl)-N- | LC/MS [Method 31]: |
| | (quinolin-5-yl)-4,5-dihydropyrazolo[1,5-a]pyrazine- | Rₜ = 1.17 min; MS |
| | 2-carboxamide 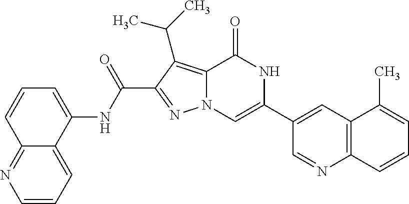 [SMILES: Cc1cccc2ncc(-c3cn4nc(C(=O)Nc5cccc6ncccc56)c(C(C)C)c4c(=O)[nH]3)cc12] | (ESIpos): m/z = 489 [M + H]⁺. |
| | (33% of theory, 99% purity) | |
| 4-30 | 3-Isopropyl-N-[2-methyl-3-(1H-pyrazol-1-yl)propyl]-6- | LC/MS [Method 31]: |
| | (5-methylquinolin-3-yl)-4-oxo-4,5-dihydropyrazolo- | Rₜ = 1.13 min; MS |
| | [1,5-a]pyrazine-2-carboxamide 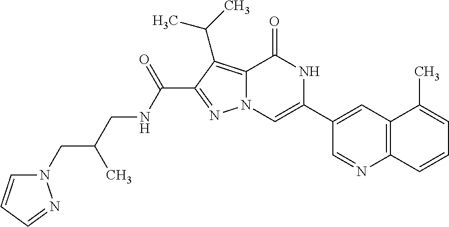 [SMILES: Cc1cccc2ncc(-c3cn4nc(C(=O)NCC(C)Cn5cccn5)c(C(C)C)c4c(=O)[nH]3)cc12] | (ESIpos): m/z = 484 [M + H]⁺. |
| | (51% of theory, 100% purity) | |
| 4-31 | N-(2-Amino-2-oxoethyl)-6-(5-methylquinolin-3-yl)-4- | LC/MS [Method 31]: |
| | oxo-3-(propan-2-yl)-4,5-dihydropyrazolo[1,5-a]- | Rₜ = 0.93 min; MS |
| | pyrazine-2-carboxamide 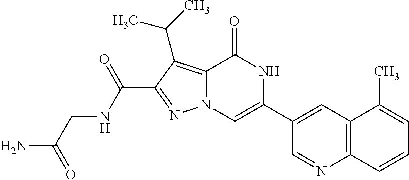 [SMILES: Cc1cccc2ncc(-c3cn4nc(C(=O)NCC(N)=O)c(C(C)C)c4c(=O)[nH]3)cc12] | (ESIpos): m/z = 419 [M + H]⁺. |
| | (9% of theory, 100% purity) | |
| 4-32 | N-(trans-4-Hydroxycyclohexyl)-3-isopropyl-6-(5- | LC/MS [Method 31]: |
| | methylquinolin-3-yl)-4-oxo-4,5-dihydropyrazolo[1,5-a]- | Rₜ = 1.01 min; MS |
| | pyrazine-2-carboxamide 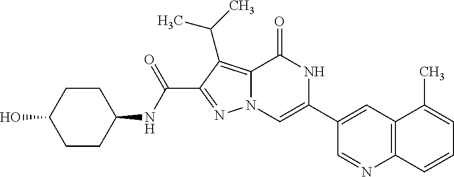 [SMILES: Cc1cccc2ncc(-c3cn4nc(C(=O)N[C@H]5CC[C@H](O)CC5)c(C(C)C)c4c(=O)[nH]3)cc12] | (ESIpos): m/z = 460 [M + H]⁺. |
| | (37% of theory, 100% purity) | |
| 4-33 | N-(2,2-Dimethylpropyl)-6-(5-methylquinolin-3-yl)-4-oxo- | LC/MS [Method 31]: |
| | 3-(propan-2-yl)-4,5-dihydropyrazolo[1,5-a]pyrazine- | Rₜ = 1.24 min; MS |
| | 2-carboxamide 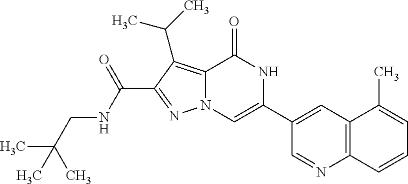 [SMILES: Cc1cccc2ncc(-c3cn4nc(C(=O)NCC(C)(C)C)c(C(C)C)c4c(=O)[nH]3)cc12] | (ESIpos): m/z = 432 [M + H]⁺. |
| | (49% of theory, 100% purity) | |
| 4-34 | N-[1-(1-Ethyl-1H-pyrazol-5-yl)ethyl]-3-isopropyl-6- | LC/MS [Method 31]: |
| | (5-methylquinolin-3-yl)-4-oxo-4,5-dihydropyrazolo- | Rₜ = 1.16 min; MS |
| | [1,5-a]pyrazine-2-carboxamide 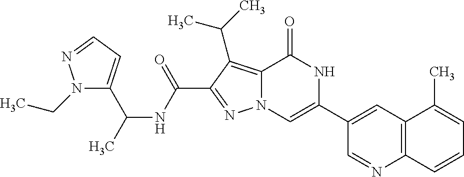 [SMILES: CCn1nccc1C(C)NC(=O)c1nn2cc(-c3cnc4cccc(C)c4c3)[nH]c(=O)c2c1C(C)C] | (ESIpos): m/z = 484 [M + H]⁺. |
| | (65% of theory, 98% purity) | |
| 4-35 | N-[3-(Methansulfonyl)propyl]-6-(5-methylquinolin-3-yl)- | LC/MS [Method 31]: |
| | 4-oxo-3-(propan-2-yl)-4,5-dihydropyrazolo[1,5-a]- | Rₜ = 0.99 min; MS |
| | pyrazine-2-carboxamide 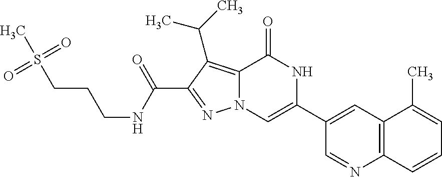 [SMILES: Cc1cccc2ncc(-c3cn4nc(C(=O)NCCCS(C)(=O)=O)c(C(C)C)c4c(=O)[nH]3)cc12] | (ESIpos): m/z = 482 [M + H]⁺. |
| | (44% of theory, 100% purity) | |
| 4-36 | N-[(1-Ethyl-3-methyl-1H-pyrazol-4-yl)methyl]-6- | LC/MS [Method 31]: |
| | (5-methylquinolin-3-yl)-4-oxo-3-(propan-2-yl)-4,5- | Rₜ = 1.12 min; MS |
| | dihydropyrazolo[1,5-a]pyrazine-2-carboxamide 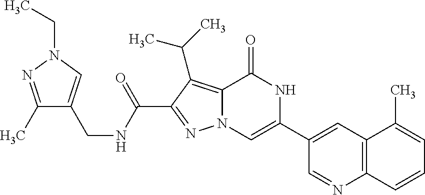 [SMILES: CCn1cc(CNC(=O)c2nn3cc(-c4cnc5cccc(C)c5c4)[nH]c(=O)c3c2C(C)C)c(C)n1] | (ESIpos): m/z = 484 [M + H]⁺. |
| | (58% of theory, 98% purity) | |
| 4-37 | N-[(1-Methyl-1H-pyrazol-4-yl)methyl]-6-(5-methyl- | LC/MS [Method 31]: |
| | quinolin-3-yl)-4-oxo-3-(propan-2-yl)-4,5-dihydropyr- | Rₜ = 1.10 min; MS |
| | azolo[1,5-a]pyrazine-2-carboxamide 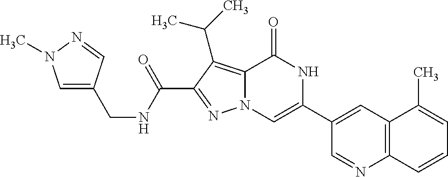 [SMILES: Cc1cccc2ncc(-c3cn4nc(C(=O)NCc5cnn(C)c5)c(C(C)C)c4c(=O)[nH]3)cc12] | (ESIpos): m/z = 456 [M + H]⁺. |
| | (18% of theory, 100% purity) | |
| 4-38 | N-(2,3-Dihydro-1H-inden-2-yl)-6-(5-methylquinolin-3- | LC/MS [Method 31]: |
| | yl)-4-oxo-3-(propan-2-yl)-4,5-dihydropyrazolo[1,5-a]- | Rₜ = 1.25 min; MS |
| | pyrazine-2-carboxamide 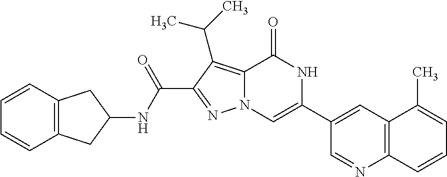 [SMILES: Cc1cccc2ncc(-c3cn4nc(C(=O)NC5Cc6ccccc6C5)c(C(C)C)c4c(=O)[nH]3)cc12] | (ESIpos): m/z = 478 [M + H]⁺. |
| | (42% of theory, 100% purity) | |
| 4-39 | 6-(5-Methylquinolin-3-yl)-4-oxo-3-(propan-2-yl)-N-[2- | LC/MS [Method 31]: |
| | (2,2,2-trifluoroethoxy)ethyl]-4,5-dihydropyrazolo[1,5-a]- | Rₜ = 1.18 min; MS |
| | pyrazine-2-carboxamide 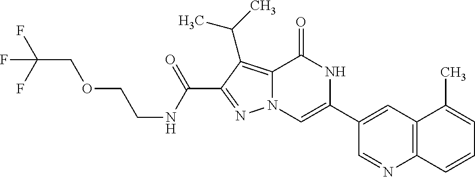 [SMILES: Cc1cccc2ncc(-c3cn4nc(C(=O)NCCOCC(F)(F)F)c(C(C)C)c4c(=O)[nH]3)cc12] | (ESIpos): m/z = 488 [M + H]⁺. |
| | (36% of theory, 100% purity) | |
| 4-40 | N-[2-(3-Cyclopropyl-1H-pyrazol-1-yl)ethyl]-6-(5-methyl- | LC/MS [Method 31]: |
| | quinolin-3-yl)-4-oxo-3-(propan-2-yl)-4,5-dihydropyr- | Rₜ = 1.15 min; MS |
| | azolo[1,5-a]pyrazine-2-carboxamide 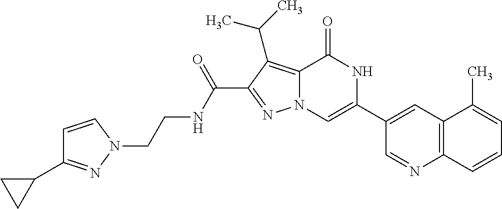 [SMILES: Cc1cccc2ncc(-c3cn4nc(C(=O)NCCn5ccc(C6CC6)n5)c(C(C)C)c4c(=O)[nH]3)cc12] | (ESIpos): m/z = 496 [M + H]⁺. |
| | (45% of theory, 100% purity) | |
| 4-41 | N-[(3-Methyl-1,2-oxazol-5-yl)methyl]-6-(5-methyl- | LC/MS [Method 31]: |
| | quinolin-3-yl)-4-oxo-3-(propan-2-yl)-4,5-dihydropyr- | Rₜ = 1.12 min; MS |
| | azolo[1,5-a]pyrazine-2-carboxamide 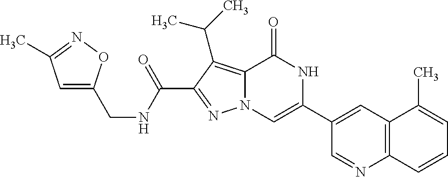 [SMILES: Cc1cc(CNC(=O)c2nn3cc(-c4cnc5cccc(C)c5c4)[nH]c(=O)c3c2C(C)C)on1] | (ESIpos): m/z = 457 [M + H]⁺. |
| | (80% of theory, 100% purity) | |
| 4-42 | 3-Isopropyl-6-(5-methyl-3-quinolyl)-4-oxo-N-[(2-oxo- | LC/MS [Method 31]: |
| | oxazolidin-5-yl)methyl]-5H-pyrazolo[1,5-a]pyrazine- | Rₜ = 0.96 min; MS |
| | 2-carboxamide 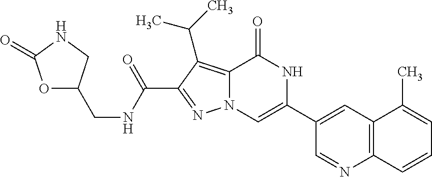 [SMILES: Cc1cccc2ncc(-c3cn4nc(C(=O)NCC5CNC(=O)O5)c(C(C)C)c4c(=O)[nH]3)cc12] | (ESIpos): m/z = 461 [M + H]⁺. |
| | (28% of theory, 100% purity) | |
| 4-43 | 6-(5-Methylquinolin-3-yl)-4-oxo-N-(1-phenylcyclo- | LC/MS [Method 31]: |
| | propyl)-3-(propan-2-yl)-4,5-dihydropyrazolo[1,5-a]- | Rₜ = 1.23 min; MS |
| | pyrazine-2-carboxamide 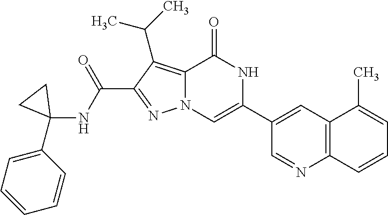 [SMILES: Cc1cccc2ncc(-c3cn4nc(C(=O)NC5(c6ccccc6)CC5)c(C(C)C)c4c(=O)[nH]3)cc12] | (ESIpos): m/z = 478 [M + H]⁺. |
| | (61% of theory, 100% purity) | |
| 4-44 | N-[1-(2-Chlorophenyl)-3-hydroxypropyl]-3-isopropyl-6- | LC/MS [Method 31]: |
| | (5-methyl-3-quinolyl)-4-oxo-5H-pyrazolo[1,5-a]- | Rₜ = 1.17 min; MS |
| | pyrazine-2-carboxamide 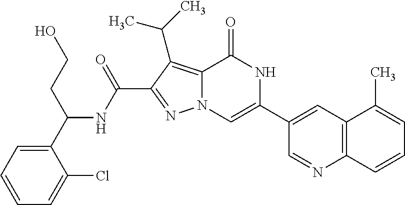 [SMILES: Cc1cccc2ncc(-c3cn4nc(C(=O)NC(CCO)c5ccccc5Cl)c(C(C)C)c4c(=O)[nH]3)cc12] | (ESIpos): m/z = 530 [M + H]⁺. |
| | (13% of theory, 100% purity) | |
| 4-45 | N-[3-Hydroxy-1-(3-methoxyphenyl)propyl]-3-isopropyl- | LC/MS [Method 31]: |
| | 6-(5-methyl-3-quinolyl)-4-oxo-5H-pyrazolo[1,5-a]- | Rₜ = 1.12 min; MS |
| | pyrazine-2-carboxamide 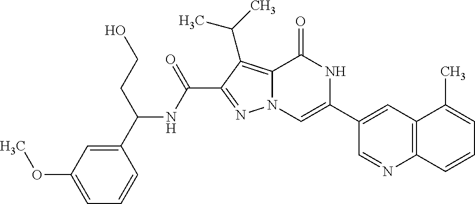 [SMILES: COc1cccc(C(CCO)NC(=O)c2nn3cc(-c4cnc5cccc(C)c5c4)[nH]c(=O)c3c2C(C)C)c1] | (ESIpos): m/z = 526 [M + H]⁺. |
| | (68% of theory, 97% purity) | |
| 4-46 | N-{1-[2-(Difluoromethoxy)phenyl]ethyl}-3-isopropyl-6- | LC/MS [Method 31]: |
| | (5-methylquinolin-3-yl)-4-oxo-4,5-dihydropyrazolo- | Rₜ = 1.26 min; MS |
| | [1,5-a]pyrazine-2-carboxamide 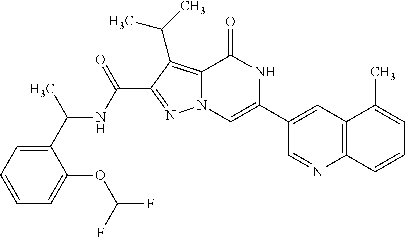 [SMILES: Cc1cccc2ncc(-c3cn4nc(C(=O)NC(C)c5ccccc5OC(F)F)c(C(C)C)c4c(=O)[nH]3)cc12] | (ESIpos): m/z = 532 [M + H]⁺. |
| | (60% of theory, 100% purity) | |
| 4-47 | 3-Isopropyl-N-[1-(4-methylpyrimidin-2-yl)ethyl]-6- | LC/MS [Method 31]: |
| | (5-methyl-3-quinolyl)-4-oxo-5H-pyrazolo[1,5-a]- | Rₜ = 1.15 min; MS |
| | pyrazine-2-carboxamide 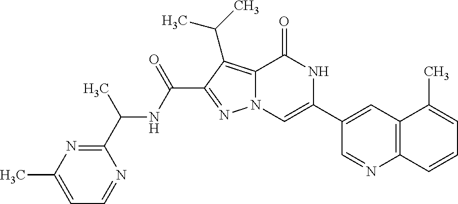 [SMILES: Cc1ccnc(C(C)NC(=O)c2nn3cc(-c4cnc5cccc(C)c5c4)[nH]c(=O)c3c2C(C)C)n1] | (ESIpos): m/z = 482 [M + H]⁺. |
| | (48% of theory, 96% purity) | |
| 4-48 | 6-(5-Methylquinolin-3-yl)-4-oxo-3-(propan-2-yl)-N- | LC/MS [Method 31]: |
| | ({4-[(pyrrolidin-1-yl)methyl]phenyl}methyl)-4,5-dihydro- | Rₜ = 0.83 min; MS |
| | pyrazolo[1,5-a]pyrazine-2-carboxamide 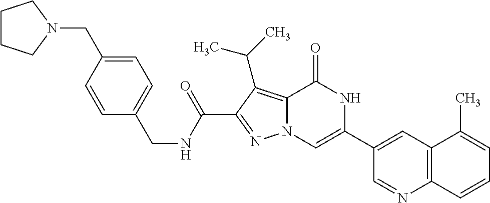 [SMILES: Cc1cccc2ncc(-c3cn4nc(C(=O)NCc5ccc(CN6CCCC6)cc5)c(C(C)C)c4c(=O)[nH]3)cc12] | (ESIpos): m/z = 535 [M + H]⁺. |
| | (33% of theory, 100% purity) | |
| 4-49 | 6-(5-Methylquinolin-3-yl)-4-oxo-N-[(1R)-1-phenyl- | LC/MS [Method 31]: |
| | propyl]-3-(propan-2-yl)-4,5-dihydropyrazolo[1,5-a]- | Rₜ = 1.26 min; MS |
| | pyrazine-2-carboxamide 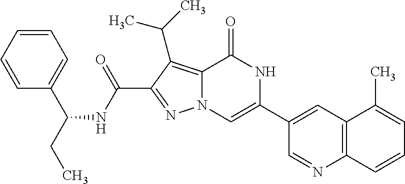 [SMILES: CC[C@@H](NC(=O)c1nn2cc(-c3cnc4cccc(C)c4c3)[nH]c(=O)c2c1C(C)C)c1ccccc1] | (ESIpos): m/z = 480 [M + H]⁺. |
| | (33% of theory, 100% purity) | |
| 4-50 | N-[1-(4-Chlorophenyl)cyclobutyl]-6-(5-methylquinolin- | LC/MS [Method 31]: |
| | 3-yl)-4-oxo-3-(propan-2-yl)-4,5-dihydropyrazolo[1,5-a]- | Rₜ = 1.30 min; MS |
| | pyrazine-2-carboxamide 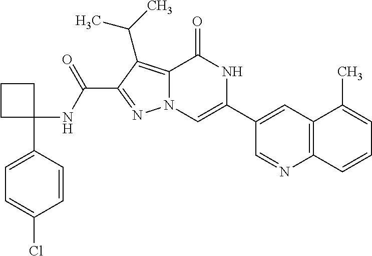 [SMILES: Cc1cccc2ncc(-c3cn4nc(C(=O)NC5(c6ccc(Cl)cc6)CCC5)c(C(C)C)c4c(=O)[nH]3)cc12] | (ESIpos): m/z = 526 [M + H]⁺. |
| | (30% of theory, 100% purity) | |
| 4-51 | N-[1-(4-Fluorophenyl)-2-methylpropyl]-3-isopropyl-6- | LC/MS [Method 31]: |
| | (5-methyl-3-quinolyl)-4-oxo-5H-pyrazolo[1,5-a]- | Rₜ = 1.28 min; MS |
| | pyrazine-2-carboxamide 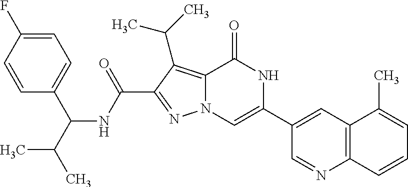 [SMILES: Cc1cccc2ncc(-c3cn4nc(C(=O)NC(c5ccc(F)cc5)C(C)C)c(C(C)C)c4c(=O)[nH]3)cc12] | (ESIpos): m/z = 512 [M + H]⁺. |
| | (43% of theory, 100% purity) | |
| 4-52 | N-[1-(3,4-Dichlorophenyl)ethyl]-3-isopropyl-6-(5- | LC/MS [Method 31]: |
| | methyl-3-quinolyl)-4-oxo-5H-pyrazolo[1,5-a]pyrazine- | Rₜ = 1.29 min; MS |
| | 2-carboxamide 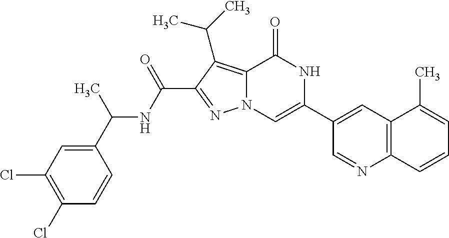 [SMILES: Cc1cccc2ncc(-c3cn4nc(C(=O)NC(C)c5ccc(Cl)c(Cl)c5)c(C(C)C)c4c(=O)[nH]3)cc12] | (ESIpos): m/z = 534 [M + H]⁺. |
| | (58% of theory, 100% purity) | |
| 4-53 | 3-Isopropyl-6-(5-methyl-3-quinolyl)-4-oxo-N-[1-(4- | LC/MS [Method 31]: |
| | sulfamoylphenyl)ethyl]-5H-pyrazolo[1,5-a]pyrazine- | Rₜ = 1.07 min; MS |
| | 2-carboxamide 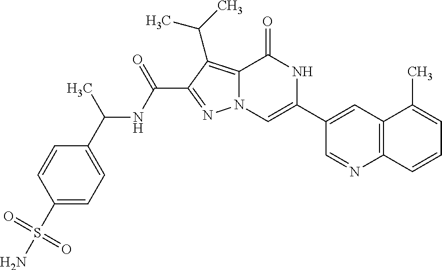 [SMILES: Cc1cccc2ncc(-c3cn4nc(C(=O)NC(C)c5ccc(S(N)(=O)=O)cc5)c(C(C)C)c4c(=O)[nH]3)cc12] | (ESIpos): m/z = 545 [M + H]⁺. |
| | (46% of theory, 100% purity) | |
| 4-54 | 3-Isopropyl-6-(5-methyl-3-quinolyl)-4-oxo-N-[1-(2- | LC/MS [Method 31]: |
| | pyridyl)ethyl]-5H-pyrazolo[1,5-a]pyrazine-2-carbox- | Rₜ = 1.08 min; MS |
| | amide 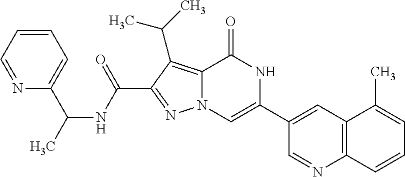 [SMILES: Cc1cccc2ncc(-c3cn4nc(C(=O)NC(C)c5ccccn5)c(C(C)C)c4c(=O)[nH]3)cc12] | (ESIpos): m/z = 467 [M + H]⁺. |
| | (59% of theory, 100% purity) | |
| 4-55 | N-[(1R)-1-(4-Methylphenyl)ethyl]-6-(5-methylquinolin- | LC/MS [Method 31]: |
| | 3-yl)-4-oxo-3-(propan-2-yl)-4,5-dihydropyrazolo[1,5-a]- | Rₜ = 1.26 min; MS |
| | pyrazine-2-carboxamide 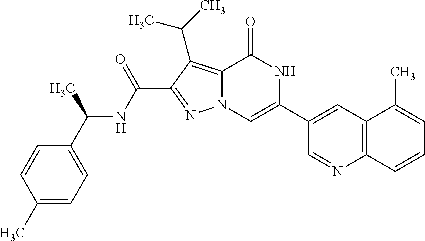 [SMILES: Cc1ccc([C@@H](C)NC(=O)c2nn3cc(-c4cnc5cccc(C)c5c4)[nH]c(=O)c3c2C(C)C)cc1] | (ESIpos): m/z = 480 [M + H]⁺. |
| | (43% of theory, 100% purity) | |
| 4-56 | N-[(7S)-1-(4-Methylphenyl)ethyl]-6-(5-methylquinolin- | LC/MS [Method 31]: |
| | 3-yl)-4-oxo-3-(propan-2-yl)-4,5-dihydropyrazolo[1,5-a]- | Rₜ = 1.26 min; MS |
| | pyrazine-2-carboxamide 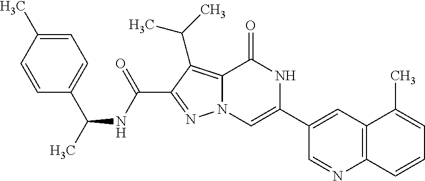 [SMILES: Cc1ccc([C@H](C)NC(=O)c2nn3cc(-c4cnc5cccc(C)c5c4)[nH]c(=O)c3c2C(C)C)cc1] | (ESIpos): m/z = 480 [M + H]⁺. |
| | (47% of theory, 100% purity) | |
| 4-57 | N-[1-(2,3-Dihydro-1,4-benzodioxin-6-yl)ethyl]-3- | LC/MS [Method 31]: |
| | isopropyl-6-(5-methyl-3-quinolyl)-4-oxo-5H-pyrazolo- | Rₜ = 1.22 min; MS |
| | [1,5-a]pyrazine-2-carboxamide 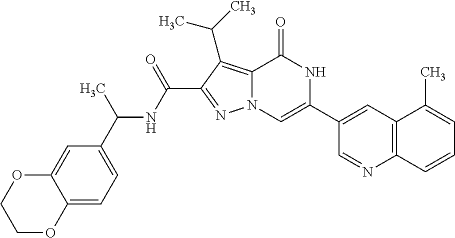 [SMILES: Cc1cccc2ncc(-c3cn4nc(C(=O)NC(C)c5ccc6c(c5)OCCO6)c(C(C)C)c4c(=O)[nH]3)cc12] | (ESIpos): m/z = 524 [M + H]⁺. |
| | (45% of theory, 100% purity) | |
| 4-58 | 6-(5-Methylquinolin-3-yl)-4-oxo-3-(propan-2-yl)-N- | LC/MS [Method 31]: |
| | [(7S)-1,2,3,4-tetrahydronaphthalen-1-yl]-4,5-dihydro- | Rₜ = 1.28 min; MS |
| | pyrazolo[1,5-a]pyrazine-2-carboxamide 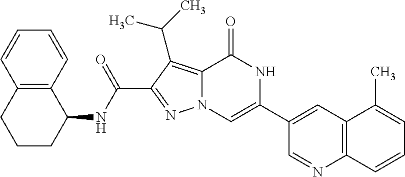 [SMILES: Cc1cccc2ncc(-c3cn4nc(C(=O)N[C@H]5CCCc6ccccc65)c(C(C)C)c4c(=O)[nH]3)cc12] | (ESIpos): m/z = 492 [M + H]⁺. |
| | (58% of theory, 100% purity) | |
| 4-59 | 6-(5-Methylquinolin-3-yl)-4-oxo-N-[(7S)-1-phenylethyl]- | LC/MS [Method 31]: |
| | 3-(propan-2-yl)-4,5-dihydropyrazolo[1,5-a]pyrazine- | Rₜ = 1.23 min; MS |
| | 2-carboxamide 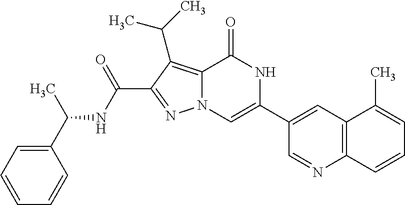 [SMILES: Cc1cccc2ncc(-c3cn4nc(C(=O)N[C@@H](C)c5ccccc5)c(C(C)C)c4c(=O)[nH]3)cc12] | (ESIpos): m/z = 466 [M + H]⁺. |
| | (42% of theory, 100% purity) | |
| 4-60 | N-[(4,6-Dimethylpyrimidin-2-yl)methyl]-6-(5-methyl- | LC/MS [Method 31]: |
| | quinolin-3-yl)-4-oxo-3-(propan-2-yl)-4,5-dihydropyr- | Rₜ = 1.15 min; MS |
| | azolo[1,5-a]pyrazine-2-carboxamide 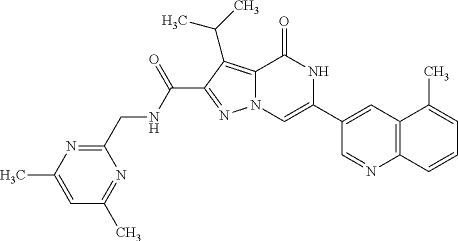 [SMILES: Cc1cc(C)nc(CNC(=O)c2nn3cc(-c4cnc5cccc(C)c5c4)[nH]c(=O)c3c2C(C)C)n1] | (ESIpos): m/z = 482 [M + H]⁺. |
| | (33% of theory, 100% purity) | |
| 4-61 | 3-Isopropyl-6-(5-methyl-3-quinolyl)-N-[1-(4-methyl- | LC/MS [Method 31]: |
| | sulfonylphenyl)ethyl]-4-oxo-5H-pyrazolo[1,5-a]- | Rₜ = 1.13 min; MS |
| | pyrazine-2-carboxamide 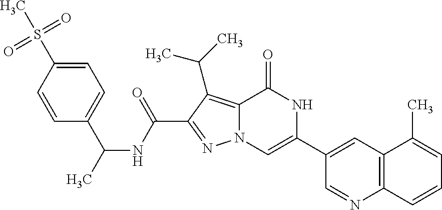 [SMILES: Cc1cccc2ncc(-c3cn4nc(C(=O)NC(C)c5ccc(S(C)(=O)=O)cc5)c(C(C)C)c4c(=O)[nH]3)cc12] | (ESIpos): m/z = 544 [M + H]⁺. |
| | (49% of theory, 98% purity) | |
| 4-62 | N-[1-(3-Bromophenyl)ethyl]-3-isopropyl-6-(5-methyl- | LC/MS [Method 31]: |
| | 3-quinolyl)-4-oxo-5H-pyrazolo[1,5-a]pyrazine- | Rₜ = 1.27 min; MS |
| | 2-carboxamide 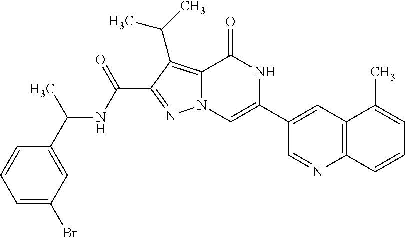 [SMILES: Cc1cccc2ncc(-c3cn4nc(C(=O)NC(C)c5cccc(Br)c5)c(C(C)C)c4c(=O)[nH]3)cc12] | (ESIpos): m/z = 544 [M + H]⁺. |
| | (48% of theory, 100% purity) | |
| 4-63 | 3-Isopropyl-6-(5-methylquinolin-3-yl)-4-oxo-N-[1- | LC/MS [Method 31]: |
| | phenyl-2-(pyrrolidin-1-yl)ethyl]-4,5-dihydropyrazolo- | Rₜ = 0.86 min; MS |
| | [1,5-a]pyrazine-2-carboxamide 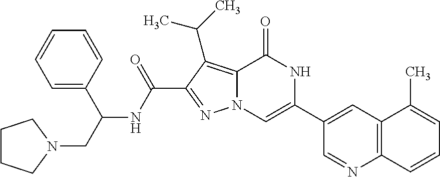 [SMILES: Cc1cccc2ncc(-c3cn4nc(C(=O)NC(CN5CCCC5)c5ccccc5)c(C(C)C)c4c(=O)[nH]3)cc12] | (ESIpos): m/z = 535 [M + H]⁺. |
| | (36% of theory, 100% purity) | |
| 4-64 | 3-Isopropyl-N-(6-methoxy-1,2,3,4- | LC/MS [Method 31]: |
| | tetrahydronaphthalen-1-yl)-6-(5-methylquinolin-3-yl)-4- | Rₜ = 1.28 min; MS |
| | oxo-4,5-dihydropyrazolo[1,5-a]pyrazine-2-carboxamide 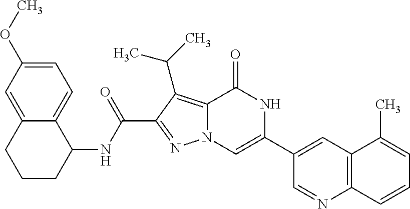 [SMILES: COc1ccc2c(c1)CCCC2NC(=O)c1nn2cc(-c3cnc4cccc(C)c4c3)[nH]c(=O)c2c1C(C)C] | (ESIpos): m/z = 522 [M + H]⁺. |
| | (46% of theory, 100% purity) | |
| 4-65 | N-(3,4-Dihydro-2H-chromen-4-yl)-3-isopropyl-6- | LC/MS [Method 31]: |
| | (5-methylquinolin-3-yl)-4-oxo-4,5-dihydropyrazolo- | Rₜ = 1.23 min; MS |
| | [1,5-a]pyrazine-2-carboxamide 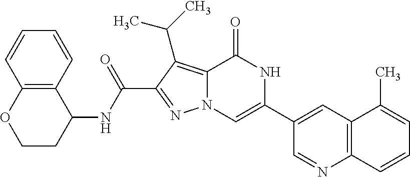 [SMILES: Cc1cccc2ncc(-c3cn4nc(C(=O)NC5CCOc6ccccc65)c(C(C)C)c4c(=O)[nH]3)cc12] | (ESIpos): m/z = 494 [M + H]⁺. |
| | (66% of theory, 99% purity) | |
| 4-66 | N-[1-(3,5-Dichlorophenyl)-2-hydroxyethyl]-3-isopropyl- | LC/MS [Method 31]: |
| | 6-(5-methyl-3-quinolyl)-4-oxo-5H-pyrazolo[1,5-a]- | Rₜ = 1.21 min; MS |
| | pyrazine-2-carboxamide 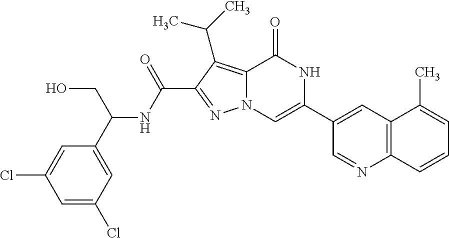 [SMILES: Cc1cccc2ncc(-c3cn4nc(C(=O)NC(CO)c5cc(Cl)cc(Cl)c5)c(C(C)C)c4c(=O)[nH]3)cc12] | (ESIpos): m/z = 550 [M + H]⁺. |
| | (33% of theory, 100% purity) | |
| 4-67 | N-[(7R)-2-Hydroxy-1-phenylethyl]-6-(5-methylquinolin- | LC/MS [Method 31]: |
| | 3-yl)-4-oxo-3-(propan-2-yl)-4,5-dihydropyrazolo[1,5-a]- | Rₜ = 1.11 min; MS |
| | pyrazine-2-carboxamide 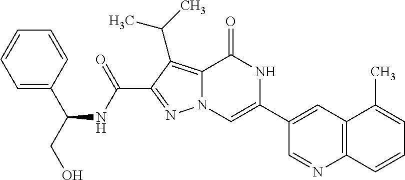 [SMILES: Cc1cccc2ncc(-c3cn4nc(C(=O)N[C@@H](CO)c5ccccc5)c(C(C)C)c4c(=O)[nH]3)cc12] | (ESIpos): m/z = 482 [M + H]⁺. |
| | (66% of theory, 100% purity) | |
| 4-68 | N-(2,2-Difluoro-1-phenylethyl)-3-isopropyl-6-(5-methyl- | LC/MS [Method 31]: |
| | 3-quinolyl)-4-oxo-5H-pyrazolo[1,5-a]pyrazine- | Rₜ = 1.24 min; MS |
| | 2-carboxamide 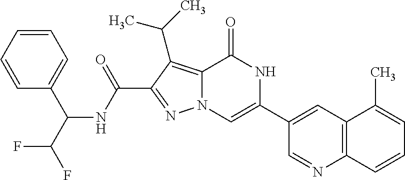 [SMILES: Cc1cccc2ncc(-c3cn4nc(C(=O)NC(c5ccccc5)C(F)F)c(C(C)C)c4c(=O)[nH]3)cc12] | (ESIpos): m/z = 502 [M + H]⁺. |
| | (41% of theory, 94% purity) | |
| 4-69 | N-[(7S,2R)-2-Hydroxy-2,3-dihydro-1H-inden-1-yl]-6- | LC/MS [Method 31]: |
| | (5-methylquinolin-3-yl)-4-oxo-3-(propan-2-yl)-4,5- | Rₜ = 1.16 min; MS |
| | dihydropyrazolo[1,5-a]pyrazine-2-carboxamide 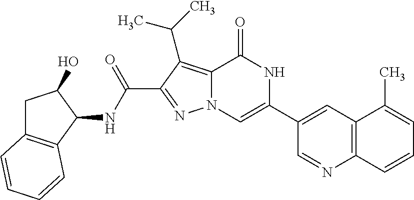 [SMILES: Cc1cccc2ncc(-c3cn4nc(C(=O)N[C@H]5c6ccccc6C[C@H]5O)c(C(C)C)c4c(=O)[nH]3)cc12] | (ESIpos): m/z = 494 [M + H]⁺. |
| | (44% of theory, 100% purity) | |
| 4-70 | 3-Isopropyl-6-(5-methyl-3-quinolyl)-4-oxo-N-[1-(3- | LC/MS [Method 31]: |
| | pyridyl)ethyl]-5H-pyrazolo[1,5-a]pyrazine-2-carbox- | Rₜ = 0.95 min; MS |
| | amide 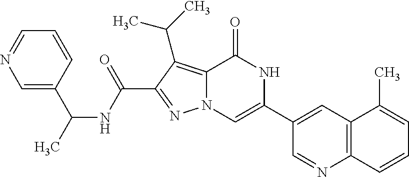 [SMILES: Cc1cccc2ncc(-c3cn4nc(C(=O)NC(C)c5cccnc5)c(C(C)C)c4c(=O)[nH]3)cc12] | (ESIpos): m/z = 467 [M + H]⁺. |
| | (32% of theory, 100% purity) | |
| 4-71 | N-[1-(3-Fluoro-4-methoxyphenyl)ethyl]-3-isopropyl-6- | LC/MS [Method 31]: |
| | (5-methyl-3-quinolyl)-4-oxo-5H-pyrazolo[1,5-a]- | Rₜ = 1.23 min; MS |
| | pyrazine-2-carboxamide 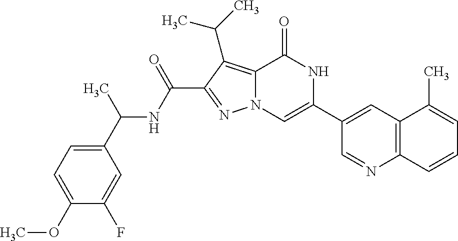 [SMILES: COc1ccc(C(C)NC(=O)c2nn3cc(-c4cnc5cccc(C)c5c4)[nH]c(=O)c3c2C(C)C)cc1F] | (ESIpos): m/z = 514 [M + H]⁺. |
| | (62% of theory, 100% purity) | |
| 4-72 | N-(2,3-Dihydro-1H-inden-1-yl)-3-isopropyl-6-(5-methyl- | LC/MS [Method 31]: |
| | quinolin-3-yl)-4-oxo-4,5-dihydropyrazolo[1,5-a]- | Rₜ = 1.26 min; MS |
| | pyrazine-2-carboxamide 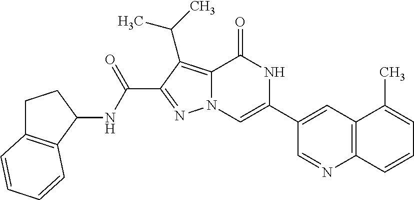 [SMILES: Cc1cccc2ncc(-c3cn4nc(C(=O)NC5CCc6ccccc65)c(C(C)C)c4c(=O)[nH]3)cc12] | (ESIpos): m/z = 478 [M + H]⁺. |
| | (6% of theory, 100% purity) | |
| 4-73 | 6-(5-Methylquinolin-3-yl)-4-oxo-N-(1-phenylcyclo- | LC/MS [Method 31]: |
| | pentyl)-3-(propan-2-yl)-4,5-dihydropyrazolo[1,5-a]- | Rₜ = 1.29 min; MS |
| | pyrazine-2-carboxamide 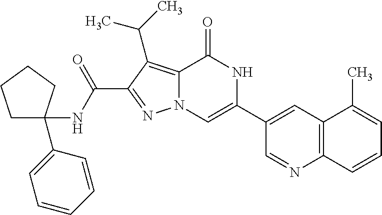 [SMILES: Cc1cccc2ncc(-c3cn4nc(C(=O)NC5(c6ccccc6)CCCC5)c(C(C)C)c4c(=O)[nH]3)cc12] | (ESIpos): m/z = 506 [M + H]⁺. |
| | (37% of theory, 100% purity) | |
| 4-74 | 3-Isopropyl-N-(6-methoxy-2,2-dimethyl-3,4-dihydro- | LC/MS [Method 31]: |
| | 2H-chromen-4-yl)-6-(5-methylquinolin-3-yl)-4-oxo-4,5- | Rₜ = 1.27 min; MS |
| | dihydropyrazolo[1,5-a]pyrazine-2-carboxamide 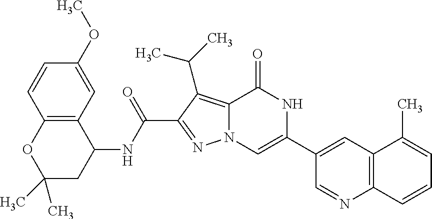 [SMILES: COc1ccc2c(c1)C(NC(=O)c1nn3cc(-c4cnc5cccc(C)c5c4)[nH]c(=O)c3c1C(C)C)CC(C)(C)O2] | (ESIpos): m/z = 552 [M + H]⁺. |
| | (48% of theory, 100% purity) | |
| 4-75 | N-[1-(4-Bromophenyl)-2-hydroxyethyl]-3-isopropyl-6- | LC/MS [Method 31]: |
| | (5-methyl-3-quinolyl)-4-oxo-5H-pyrazolo[1,5-a]- | Rₜ = 1.16 min; MS |
| | pyrazine-2-carboxamide 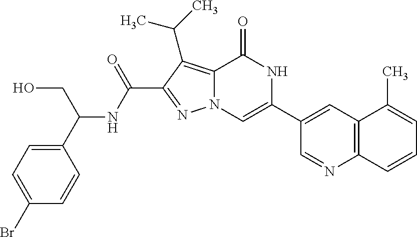 [SMILES: Cc1cccc2ncc(-c3cn4nc(C(=O)NC(CO)c5ccc(Br)cc5)c(C(C)C)c4c(=O)[nH]3)cc12] | (ESIpos): m/z = 560 [M + H]⁺. |
| | (21% of theory, 100% purity) | |
| 4-76 | N-[2,2-Difluoro-1-(3-fluorophenyl)ethyl]-3-isopropyl-6- | LC/MS [Method 31]: |
| | (5-methyl-3-quinolyl)-4-oxo-5H-pyrazolo[1,5-a]- | Rₜ = 1.24 min; MS |
| | pyrazine-2-carboxamide 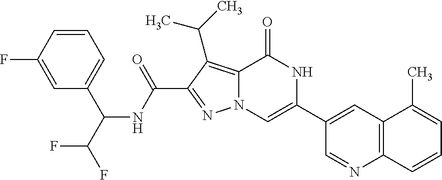 [SMILES: Cc1cccc2ncc(-c3cn4nc(C(=O)NC(c5cccc(F)c5)C(F)F)c(C(C)C)c4c(=O)[nH]3)cc12] | (ESIpos): m/z = 520 [M + H]⁺. |
| | (51% of theory, 100% purity) | |
| 4-77 | 3-Isopropyl-6-(5-methylquinolin-3-yl)-4-oxo-N-(4- | LC/MS [Method 31]: |
| | phenyltetrahydro-2H-pyran-4-yl)-4,5-dihydropyrazolo- | Rₜ = 1.22 min; MS |
| | [1,5-a]pyrazine-2-carboxamide 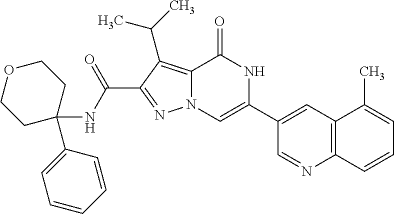 [SMILES: Cc1cccc2ncc(-c3cn4nc(C(=O)NC5(c6ccccc6)CCOCC5)c(C(C)C)c4c(=O)[nH]3)cc12] | (ESIpos): m/z = 522 [M + H]⁺. |
| | (47% of theory, 100% purity) | |
| 4-78 | N-[Cyclopropyl(4-methoxyphenyl)methyl]-3-isopropyl- | LC/MS [Method 31]: |
| | 6-(5-methyl-3-quinolyl)-4-oxo-5H-pyrazolo[1,5-a]- | Rₜ = 1.25 min; MS |
| | pyrazine-2-carboxamide 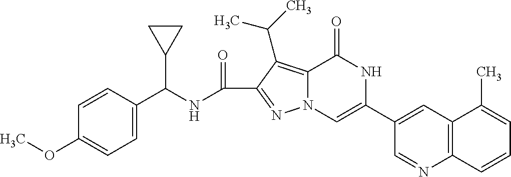 [SMILES: COc1ccc(C(NC(=O)c2nn3cc(-c4cnc5cccc(C)c5c4)[nH]c(=O)c3c2C(C)C)C2CC2)cc1] | (ESIpos): m/z = 522 [M + H]⁺. |
| | (36% of theory, 100% purity) | |
| 4-79 | 3-Isopropyl-N-(7-methoxy-2,2-dimethyl-3,4-dihydro- | LC/MS [Method 31]: |
| | 2-chromen-4-yl)-6-(5-methylquinolin-3-yl)-4-oxo-4,5- | Rₜ = 1.28 min; MS |
| | dihydropyrazolo[1,5-a]pyrazine-2-carboxamide 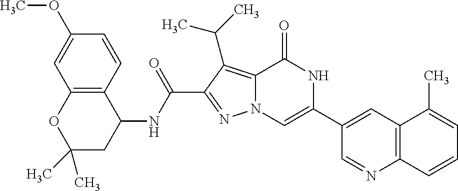 [SMILES: COc1ccc2c(c1)OC(C)(C)CC2NC(=O)c1nn2cc(-c3cnc4cccc(C)c4c3)[nH]c(=O)c2c1C(C)C] | (ESIpos): m/z = 552 [M + H]⁺. |
| | (66% of theory, 97% purity) | |
| 4-80 | 6-(5-Methylquinolin-3-yl)-4-oxo-N-[(2-oxo-2,3-dihydro- | LC/MS [Method 31]: |
| | 1H-benzimidazol-5-yl)methyl]-3-(propan-2-yl)-4,5- | Rₜ = 1.02 min; MS |
| | dihydropyrazolo[1,5-a]pyrazine-2-carboxamide 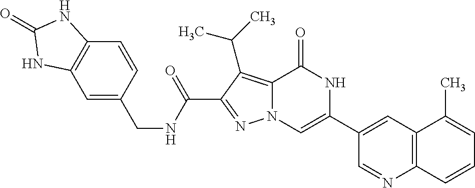 [SMILES: Cc1cccc2ncc(-c3cn4nc(C(=O)NCc5ccc6[nH]c(=O)[nH]c6c5)c(C(C)C)c4c(=O)[nH]3)cc12] | (ESIpos): m/z = 508 [M + H]⁺. |
| | (17% of theory, 97% purity) | |
| 4-81 | 6-(5-Methylquinolin-3-yl)-4-oxo-3-(propan-2-yl)-N- | LC/MS [Method 31]: |
| | ({3-[(pyrrolidin-1-yl)methyl]phenyl}methyl)-4,5-dihydro- | Rₜ = 0.84 min; MS |
| | pyrazolo[1,5-a]pyrazine-2-carboxamide 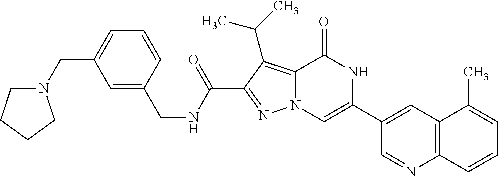 [SMILES: Cc1cccc2ncc(-c3cn4nc(C(=O)NCc5cccc(CN6CCCC6)c5)c(C(C)C)c4c(=O)[nH]3)cc12] | (ESIpos): m/z = 535 [M + H]⁺. |
| | (47% of theory, 93% purity) | |
| 4-82 | N-[1-(2,4-Dichlorophenyl)ethyl]-3-isopropyl-6-(5- | LC/MS [Method 31]: |
| | methyl-3-quinolyl)-4-oxo-5H-pyrazolo[1,5-a]pyrazine- | Rₜ = 1.30 min; MS |
| | 2-carboxamide 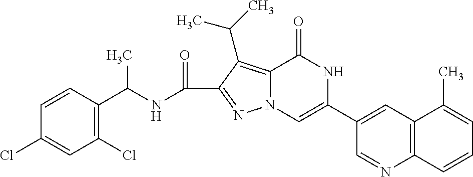 [SMILES: Cc1cccc2ncc(-c3cn4nc(C(=O)NC(C)c5ccc(Cl)cc5Cl)c(C(C)C)c4c(=O)[nH]3)cc12] | (ESIpos): m/z = 534 [M + H]⁺. |
| | (1% of theory, 100% purity) | |
| 4-83 | N-[3-Hydroxy-1-(4-methoxyphenyl)propyl]-3-isopropyl- | LC/MS [Method 31]: |
| | 6-(5-methyl-3-quinolyl)-4-oxo-5H-pyrazolo[1,5-a]- | Rₜ = 1.11 min; MS |
| | pyrazine-2-carboxamide 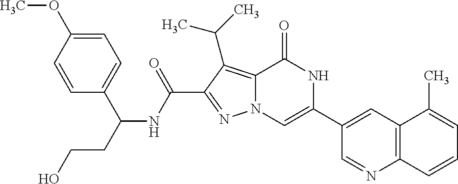 [SMILES: COc1ccc(C(CCO)NC(=O)c2nn3cc(-c4cnc5cccc(C)c5c4)[nH]c(=O)c3c2C(C)C)cc1] | (ESIpos): m/z = 526 [M + H]⁺. |
| | (66% of theory, 100% purity) | |
| 4-84 | 3-Isopropyl-6-(5-methylquinolin-3-yl)-4-oxo-N-(2,2,6- | LC/MS [Method 31]: |
| | trimethyl-3,4-dihydro-2H-chromen-4-yl)-4,5-dihydropyr- | Rₜ = 1.29 min; MS |
| | azolo[1,5-a]pyrazine-2-carboxamide 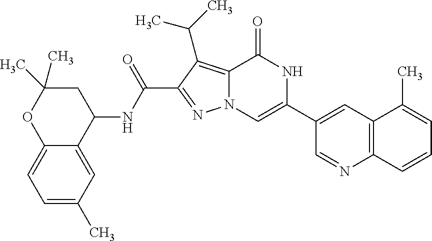 [SMILES: Cc1ccc2c(c1)C(NC(=O)c1nn3cc(-c4cnc5cccc(C)c5c4)[nH]c(=O)c3c1C(C)C)CC(C)(C)O2] | (ESIpos): m/z = 536 [M + H]⁺. |
| | (45% of theory, 100% purity) | |

General Procedure 5 (Parallel Synthesis of Example Compounds):

6-(2,3-Dihydro-1,4-benzodioxin-6-yl)-4-oxo-4,5-dihydropyrazolo[1,5-a]pyrazine-2-carboxylic acid (Intermediate 102A, 31.3 mg, 100 μmol) or 6-(3,4-dimethylphenyl)-4-oxo-4,5-dihydropyrazolo-[1,5-a]pyrazine-2-carboxylic acid (Intermediate 23A, 28.3 mg, 100 μmol) in thionylchloride (500 μl) was heated to 60° C. overnight. The volatiles were then removed under reduced pressure, and the crude acid chloride was dissolved in 1,2-dichloroethane (800 μl) and added to the corresponding amine which was placed on a multi-well plate. N,N-Diisopropylethylamine (100 μl, 1.02 mmol) was added, and the plate was shaken at RT overnight. After this, the volatiles were removed under reduced pressure, the residue was taken up in DMF, and the mixture was filtered. The filtrate solution was directly purified by preparative HPLC (Method P11). The fractions containing the desired product were combined, evaporated and dried in vacuo.

The example compounds specified in the following Table 4 were prepared according to general procedure 5:

TABLE 4

| | IUPAC name/structure | |
|---|---|---|
| Example | (yield, purity) | Analytical data |
| 5-1 | 6-(3,4-Dimethylphenyl)-N-[1-(4-fluorophenyl)ethyl]- | LC/MS [Method 31]: |
| | 4-oxo-5H-pyrazolo[1,5-a]pyrazine-2-carboxamide 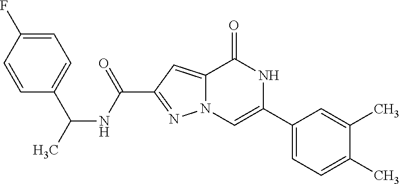 [SMILES: Cc1ccc(-c2cn3nc(C(=O)NC(C)c4ccc(F)cc4)cc3c(=O)[nH]2)cc1C] | Rₜ = 1.18 min; MS (ESIpos): m/z = 405 [M + H]⁺. |
| | (11% of theory, 92% purity) | |
| 5-2 | 6-(2,3-Dihydro-1,4-benzodioxin-6-yl)-N-[1-(4-fluoro- | LC/MS [Method 31]: |
| | phenyl)ethyl]-4-oxo-5H-pyrazolo[1,5-a]pyrazine- | Rₜ = 1.07 min; MS |
| | 2-carboxamide 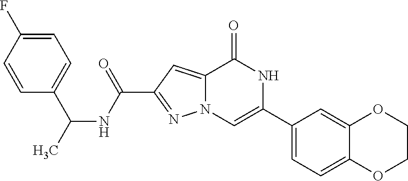 [SMILES: CC(NC(=O)c1cc2c(=O)[nH]c(-c3ccc4c(c3)OCCO4)cn2n1)c1ccc(F)cc1] | (ESIpos): m/z = 435 [M + H]⁺. |
| | (7% of theory, 99% purity) | |
| 5-3 | 6-(2,3-Dihydro-1,4-benzodioxin-6-yl)-N-[3-hydroxy-1- | LC/MS [Method 31]: |
| | (4-methoxyphenyl)propyl]-4-oxo-5H-pyrazolo[1,5-a]- | Rₜ = 0.95 min; MS |
| | pyrazine-2-carboxamide 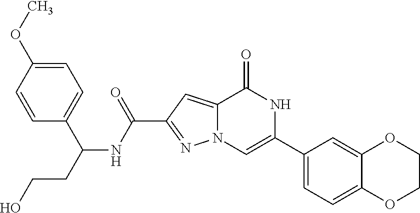 [SMILES: COc1ccc(C(CCO)NC(=O)c2cc3c(=O)[nH]c(-c4ccc5c(c4)OCCO5)cn3n2)cc1] | (ESIpos): m/z = 477 [M + H]⁺. |
| | (4% of theory, 96% purity) | |
| 5-4 | 6-(3,4-Dimethylphenyl)-N-[1-(6-methoxypyridin-3-yl)- | LC/MS [Method 31]: |
| | ethyl]-4-oxo-4,5-dihydropyrazolo[1,5-a]pyrazine- | Rₜ = 1.12 min; MS |
| | 2-carboxamide 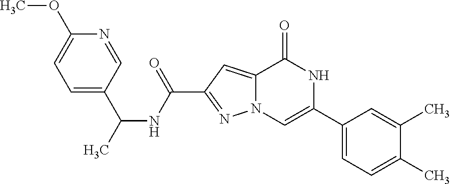 | (ESIpos): m/z = 418 [M + H]⁺. |
| | (18% of theory, 95% purity) | |
| 5-5 | 6-(3,4-Dimethylphenyl)-N-(1-methylcyclobutyl)-4-oxo- | LC/MS [Method 31]: |
| | 4,5-dihydropyrazolo[1,5-a]pyrazine-2-carboxamide 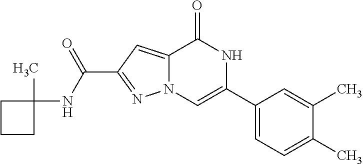 [SMILES: Cc1ccc(-c2cn3nc(C(=O)NC4(C)CCC4)cc3c(=O)[nH]2)cc1C] | Rₜ = 1.15 min; MS (ESIpos): m/z = 351 [M + H]⁺. |
| | (9% of theory, 100% purity) | |

The example compounds specified in the following Table 5 were synthesized by means of standard amide coupling reactions following standard procedures, starting from the corresponding carboxylic acid and primary amine components and using one of the following combinations of reagents:
[a] HATU, DIPEA;
[b] N-Ethyl-N′-(3-dimethylaminopropyl)carbodiimide hydrochloride (EDC), 1-hydroxy-7-azabenzotriazole (HOAt), DIPEA;
[c] Oxalyl chloride (pre-formation of the acid chloride in a separate flask), DIPEA, optional with or without DMAP;
[d] Propylphosphonic anhydride (T3P®), DIPEA;
[e] CDI, DMAP;
[f] Isobutyl chloroformate, DIPEA;
[g] Isobutyl chloroformate, N-methylmorpholine;
[h] EDC, DMAP.

Following standard work-up, purification of the respective product was achieved by preparative HPLC and/or silica gel column chromatography.

Lengthy table referenced here
US20220324865A1-20221013-T00001
Please refer to the end of the specification for access instructions.

### EXPERIMENTAL SECTION—BIOLOGICAL ASSAYS

Biological Investigations

The example testing experiments described herein serve to illustrate the present invention and the invention is not limited to the examples given.

The following assays can be used to illustrate the commercial utility of the compounds according to the present invention.

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values calculated utilizing data sets obtained from testing of one or more synthetic batch.

The in vitro activity of the compounds of the present invention can be demonstrated in the following assays.

Biological Assays

B-1. Cellular In Vitro Assay for Determining EP3 Receptor Activity

The identification of antagonists of the EP3 receptor from humans and rats as well as the quantification of the activity of the compounds of the invention was carried out using recombinant cell lines. These cell lines originally derive from a hamsters ovary epithelial cell (Chinese Hamster Ovary, CHO K1, ATCC: American Type Culture Collection, Manassas, Va. 20108, USA). The test cell lines constitutively express the human or rat EP3 receptors. The naturally G_{αi}-coupled human and rat EP3 receptors were stably co-transfected with G_{α16} into cells that are also stably transfected with a mitochondrial form of the calcium-sensitive photoproteins Clytin (human EP3) or Photina (rat EP3), which, after reconstitution with the cofactor coelenterazine, emit light when there are increases in free calcium concentrations (Nature 1992, 358, 325-327; Gene 1995, 153 (2), 273-274). The resulting EP3 receptor cells react to stimulation of the recombinantly expressed EP3 receptors by the agonist sulprostone with intracellular release of calcium ions, which can be quantified by the resulting photoprotein luminescence. Antagonists inhibit the receptor signaling, leading to reduced calcium release and luminescence. The bioluminescence of the cell lines is detected using a suitable luminometer (Trends Pharmacol. Sci. 1996, 17, 235-237).

Test Procedure:

Human and Rat EP3 Cell Lines:

On the day before the assay, the cells are plated out in culture medium (OptiMEM, 1% FCS, 2 mM glutamine, 10 mM HEPES, 5 μg/ml coelenterazine) in 384-well microtiter plates and kept in a cell incubator (96% humidity, 5% v/v CO₂, 37° C.). On the day of the assay, test compounds in various concentrations are placed for 10 minutes in the wells of the microtiter plate before the agonist sulprostone at EC₅₀ concentration is added. The resulting light signal is measured immediately in a luminometer. All concentrations are measured in quadruples.

| | EP3 CHO mtCLY ANTAG |
|---|---|
| Ex. | IC₅₀ [nmol/L] |
| 1-1 | 1.60 |
| 1-2 | 2.50 |
| 1-3 | 0.653 |
| 1-4 | 4.36 |
| 1-5 | 2.70 |
| 1-6 | 1.28 |
| 1-7 | 1.70 |
| 1-8 | 1.36 |
| 1-9 | 2.37 |
| 1-10 | 0.580 |
| 1-11 | 2.15 |
| 1-12 | 1.67 |
| 1-13 | 1.15 |
| 1-14 | 0.140 |
| 1-15 | 1.70 |
| 1-16 | 2.00 |
| 1-17 | 0.575 |
| 1-18 | 1.04 |
| 1-19 | 0.705 |
| 1-20 | 1.09 |
| 1-21 | 1.25 |
| 1-22 | 0.370 |
| 1-23 | 5.55 |
| 1-24 | 7.30 |
| 1-25 | 5.80 |
| 1-26 | 1.40 |
| 1-27 | 2.25 |
| 1-28 | 0.855 |
| 1-29 | 1.55 |
| 1-30 | 4.05 |
| 1-31 | 1.84 |
| 1-32 | 1.60 |
| 1-33 | |
| 1-34 | 4.18 |
| 1-35 | 4.25 |
| 1-36 | 2.19 |
| 1-37 | 0.950 |
| 1-38 | 4.09 |
| 1-39 | 0.265 |
| 1-40 | 8.27 |
| 1-41 | 3.70 |
| 1-42 | 1.15E⁻⁸ |
| 1-43 | 1.70 |
| 1-44 | 8.97 |
| 1-45 | 2.85 |
| 1-46 | 1.37E⁻⁸ |
| 1-47 | 4.65 |
| 1-48 | 7.60E⁻⁸ |
| 1-49 | 7.65 |
| 1-50 | 3.53 |
| 1-51 | 4.00 |
| 1-52 | 6.95 |
| 1-53 | 0.820 |
| 1-54 | 3.65 |
| 1-55 | 0.488 |
| 1-56 | 0.985 |
| 1-57 | 4.90 |
| 1-58 | 1.50 |
| 1-59 | 5.90 |
| 1-60 | 2.70 |
| 1-61 | 4.20 |
| 1-62 | 3.60 |
| 1-63 | 0.443 |
| 1-64 | 4.55 |
| 1-65 | |
| 1-66 | 1.65 |
| 1-67 | 0.905 |
| 1-68 | 1.42 |
| 1-69 | 2.25 |
| 1-70 | 3.15 |
| 1-71 | 1.45 |
| 1-72 | 6.55 |
| 1-73 | 1.30 |
| 1-74 | 0.920 |
| 1-75 | 8.75 |
| 1-76 | 0.725 |
| 1-77 | 1.18 |
| 1-78 | 1.06 |
| 1-79 | 0.600 |
| 1-80 | 6.68 |
| 1-81 | 1.59 |
| 1-82 | 4.60 |
| 1-83 | 0.523 |
| 1-84 | 1.20 |
| 1-85 | 3.00 |
| 1-86 | 0.715 |
| 1-87 | 2.85 |
| 1-88 | 1.35 |
| 1-89 | 1.03 |
| 1-90 | 0.515 |
| 1-91 | 0.915 |
| 1-92 | 0.625 |
| 1-93 | 3.58 |
| 1-94 | 0.765 |
| 1-95 | 1.70 |
| 1-96 | 2.65 |
| 1-97 | 0.880 |
| 1-98 | 3.45 |
| 1-99 | 4.00 |
| 1-100 | 0.965 |
| 1-101 | 1.70 |
| 1-102 | 1.35 |
| 1-103 | 1.87 |
| 1-104 | 2.26 |
| 1-105 | 1.48 |
| 1-106 | 5.65 |
| 1-107 | 3.97 |
| 1-108 | 2.50 |
| 1-109 | 5.97 |
| 1-110 | 1.01 |
| 1-111 | 8.80 |
| 1-112 | 3.35 |
| 1-113 | 3.80 |
| 1-114 | 3.15 |
| 1-115 | 3.40 |
| 1-116 | 9.55 |
| 1-117 | 1.38 |
| 1-118 | 3.95 |
| 1-119 | 3.30 |
| 1-120 | 0.495 |
| 1-121 | 4.18 |
| 1-122 | 4.13 |
| 1-123 | 3.20 |
| 1-124 | |
| 1-125 | 3.35 |
| 1-126 | 5.10 |
| 1-127 | 2.15 |
| 1-128 | 2.90 |
| 1-129 | 7.82 |
| 1-130 | 2.55 |
| 1-131 | 5.45 |
| 1-132 | 4.05 |
| 1-133 | 2.80 |
| 1-134 | 5.75 |
| 1-135 | 5.80 |
| 1-136 | 5.80 |
| 1-137 | 2.90 |
| 1-138 | 6.85 |
| 1-139 | 7.55 |
| 1-140 | 1.95 |
| 1-141 | 1.70 |
| 1-142 | 1.16 |
| 1-143 | 1.50 |
| 1-144 | 3.05 |
| 1-145 | 7.05 |
| 1-146 | 4.50 |
| 1-147 | 2.90 |
| 1-148 | 1.33 |
| 1-149 | 2.33 |
| 1-150 | 7.23 |
| 1-151 | 1.15 |
| 1-152 | 2.10 |
| 1-153 | 5.40 |
| 1-154 | 2.60 |
| 1-155 | 1.50 |
| 1-156 | 3.05 |
| 1-157 | 2.20 |
| 1-158 | 0.983 |
| 1-159 | 2.00 |
| 1-160 | 3.55 |
| 1-161 | 5.70 |
| 1-162 | 0.890 |
| 1-163 | 7.80 |
| 1-164 | 2.90 |
| 1-165 | |
| 1-166 | 3.15 |
| 1-167 | 2.20 |
| 1-168 | 1.90 |
| 1-169 | 3.35 |
| 1-170 | 2.50 |
| 1-171 | 1.03 |
| 1-172 | 0.86 |
| 1-173 | 0.73 |
| 1-174 | |
| 1-175 | 5.00 |
| 1-176 | 1.67 |
| 1-177 | 5.00 |
| 2-1 | 2.07 |
| 2-2 | 1.18 |
| 2-3 | 1.65 |
| 2-4 | 1.07 |
| 2-5 | 4.00 |
| 2-6 | 8.75 |
| 2-7 | 4.70 |
| 2-8 | 1.20 |
| 2-9 | 3.65 |
| 2-10 | 1.70 |
| 2-11 | 7.15 |
| 2-12 | 4.65 |
| 2-13 | 1.30 |
| 2-14 | 1.25 |
| 2-15 | 9.55 |
| 2-16 | 6.20 |
| 2-17 | 6.50 |
| 2-18 | 2.20 |
| 2-19 | 7.90 |
| 2-20 | 9.75 |
| 2-21 | 4.45 |
| 2-22 | 3.85 |
| 2-23 | 1.14 |
| 2-24 | 2.45 |
| 2-25 | 2.90 |
| 2-26 | 2.65 |
| 2-27 | 3.25 |
| 2-28 | 0.930 |
| 2-29 | 7.25 |
| 2-30 | 5.45 |
| 2-31 | 2.55 |
| 2-32 | 2.80 |
| 2-33 | 1.50 |
| 2-34 | 1.90 |
| 2-35 | 5.30 |
| 2-36 | 2.80 |
| 2-37 | 4.50 |
| 2-38 | 2.05 |
| 2-39 | 3.70 |
| 2-40 | 3.15 |
| 2-41 | 2.15 |
| 2-42 | 7.27 |
| 2-43 | 3.70 |
| 2-44 | 1.30 |
| 2-45 | 6.05 |
| 2-46 | 2.95 |
| 2-47 | 3.27 |
| 2-48 | 3.60 |
| 2-49 | 8.60 |
| 2-50 | 1.15 |
| 2-51 | 2.05 |
| 2-52 | 4.40 |
| 2-53 | 3.70 |
| 2-54 | 7.05 |
| 2-55 | 1.15 |
| 3-1 | 0.790 |
| 3-2 | 1.20 |
| 3-3 | 5.40 |
| 3-4 | 2.60 |
| 3-5 | 7.00 |
| 3-6 | 4.75 |
| 3-7 | 1.34 |
| 3-8 | 2.90 |
| 3-9 | 1.95 |
| 3-10 | 2.75 |
| 3-11 | 2.05 |
| 3-12 | 1.75 |
| 3-13 | 7.15 |
| 3-14 | 1.25 |
| 3-15 | 9.55 |
| 3-16 | 7.20 |
| 3-17 | 1.80 |
| 3-18 | 3.75 |
| 3-19 | 1.55 |
| 3-20 | 8.00 |
| 3-21 | 1.80 |
| 3-22 | 2.35 |
| 3-23 | 3.20 |
| 3-24 | 4.85 |
| 3-25 | 2.40 |
| 3-26 | 4.15 |
| 3-27 | 2.10 |
| 3-28 | 4.30 |
| 3-29 | 4.65 |
| 3-30 | 9.40 |
| 3-31 | 4.45 |
| 3-32 | 5.80 |
| 3-33 | 2.20 |
| 3-34 | 6.05 |
| 3-35 | 5.05 |
| 3-36 | 6.55 |
| 3-37 | 2.40 |
| 3-38 | 1.85 |
| 3-39 | 1.80 |
| 3-40 | 7.60 |
| 3-41 | 3.95 |
| 3-42 | 4.90 |
| 3-43 | 3.85 |
| 3-44 | 2.60 |
| 3-45 | 8.75 |
| 3-46 | 3.20 |
| 3-47 | 5.45 |
| 3-48 | 5.50 |
| 3-49 | 1.35 |
| 3-50 | 1.75 |
| 3-51 | 1.85 |
| 3-52 | 2.25 |
| 3-53 | 1.45 |
| 3-54 | 6.95 |
| 3-55 | 7.20 |
| 3-56 | 3.15 |
| 3-57 | 2.95 |
| 3-58 | 2.35 |
| 3-59 | 1.25 |
| 3-60 | 1.85 |
| 3-61 | 2.00 |
| 3-62 | 4.00 |
| 3-63 | 3.90 |
| 3-64 | 4.40 |
| 3-65 | 2.00 |
| 3-66 | 7.45 |
| 3-67 | 2.05 |
| 3-68 | 1.85 |
| 3-69 | 2.50 |
| 3-70 | 5.45 |
| 3-71 | 9.45 |
| 3-72 | 2.10 |
| 3-73 | 2.70 |
| 3-74 | 4.80 |
| 3-75 | 6.45 |
| 3-76 | 4.35 |
| 3-77 | 4.65 |
| 4-1 | 1.75 |
| 4-2 | 2.80 |
| 4-3 | 1.65 |
| 4-4 | 2.45 |
| 4-5 | 7.70 |
| 4-6 | 1.16 |
| 4-7 | 1.14 |
| 4-8 | 4.30 |
| 4-9 | 3.70 |
| 4-10 | 2.00 |
| 4-11 | 0.735 |
| 4-12 | 1.65 |
| 4-13 | 1.45 |
| 4-14 | 2.50 |
| 4-15 | 2.90 |
| 4-16 | 0.500 |
| 4-17 | 2.45 |
| 4-18 | 1.05 |
| 4-19 | 1.75 |
| 4-20 | 2.90 |
| 4-21 | 1.95 |
| 4-22 | 0.815 |
| 4-23 | 1.01 |
| 4-24 | 1.15 |
| 4-25 | 3.25 |
| 4-26 | 2.75 |
| 4-27 | 1.20 |
| 4-28 | 2.15 |
| 4-29 | 1.60 |
| 4-30 | 2.70 |
| 4-31 | 7.40 |
| 4-32 | 6.50 |
| 4-33 | 1.90 |
| 4-34 | 1.09 |
| 4-35 | 1.90 |
| 4-36 | 0.785 |
| 4-37 | 2.00 |
| 4-38 | 1.30 |
| 4-39 | 1.85 |
| 4-40 | 0.850 |
| 4-41 | 0.950 |
| 4-42 | 4.45 |
| 4-43 | 0.840 |
| 4-44 | 1.45 |
| 4-45 | 0.610 |
| 4-46 | 1.05 |
| 4-47 | 3.10 |
| 4-48 | 4.75 |
| 4-49 | 3.95 |
| 4-50 | 8.30 |
| 4-51 | 4.05 |
| 4-52 | 4.05 |
| 4-53 | 1.10 |
| 4-54 | 2.00 |
| 4-55 | 2.75 |
| 4-56 | 4.00 |
| 4-57 | 1.90 |
| 4-58 | 4.30 |
| 4-59 | 0.675 |
| 4-60 | 2.10 |
| 4-61 | 1.80 |
| 4-62 | 2.05 |
| 4-63 | 9.45 |
| 4-64 | 2.55 |
| 4-65 | 1.15 |
| 4-66 | 3.90 |
| 4-67 | 1.55 |
| 4-68 | 3.00 |
| 4-69 | 2.00 |
| 4-70 | 2.40 |
| 4-71 | 2.40 |
| 4-72 | 6.05 |
| 4-73 | 2.35 |
| 4-74 | 6.05 |
| 4-75 | 1.25 |
| 4-76 | 3.15 |
| 4-77 | 0.835 |
| 4-78 | 1.55 |
| 4-79 | 7.60 |
| 4-80 | 1.35 |
| 4-81 | 4.45 |
| 4-82 | 6.25 |
| 4-83 | 2.00 |
| 4-84 | 6.95 |
| 5-1 | 6.78 |
| 5-2 | 1.65 |
| 5-3 | 2.45 |
| 5-4 | 2.50 |
| 5-5 | 6.70 |
| 6-1 | 2.15 |
| 6-2 | 3.85 |
| 6-3 | 7.95 |
| 6-4 | 6.90 |
| 6-5 | 4.85 |
| 6-6 | 2.80 |
| 6-7 | 7.00 |
| 6-8 | 1.05 |
| 6-9 | 5.75 |
| 6-10 | 1.40 |
| 6-11 | 4.65 |
| 6-12 | 3.05 |
| 6-13 | 9.30 |
| 6-14 | 8.25 |
| 6-15 | 2.37 |
| 6-16 | 4.16 |
| 6-17 | 3.75 |
| 6-18 | 1.75 |
| 6-19 | 3.10 |
| 6-20 | 2.70 |
| 6-21 | 5.35 |
| 6-22 | 4.00 |
| 6-23 | 3.10 |
| 6-24 | 2.03 |
| 6-25 | 9.10 |
| 6-26 | 5.00 |
| 6-27 | 2.63 |
| 6-28 | 3.90 |
| 6-29 | 2.27 |
| 6-30 | 2.15 |
| 6-31 | 8.03 |
| 6-32 | 7.50 |
| 6-33 | 8.15 |
| 6-34 | 9.10 |
| 6-35 | 3.40 |
| 6-36 | 1.20 |
| 6-37 | 1.31 |
| 6-38 | 1.45 |
| 6-39 | 6.25 |
| 6-40 | 7.50 |
| 6-41 | 6.03 |
| 6-42 | 4.85 |
| 6-43 | 3.15 |
| 6-44 | 5.30 |
| 6-45 | 2.55 |
| 6-46 | 2.43 |
| 6-47 | 2.17 |
| 6-48 | 5.70 |
| 6-49 | 1.60 |
| 6-50 | 1.30 |
| 6-51 | 2.65 |
| 6-52 | 3.55 |
| 6-53 | 1.95 |
| 6-54 | 3.35 |
| 6-55 | 1.53 |
| 6-56 | 0.740 |
| 6-57 | 1.65 |
| 6-58 | 2.30 |
| 6-59 | 4.20 |
| 6-60 | 9.57 |
| 6-61 | 4.13 |
| 6-62 | 3.13 |
| 6-63 | 7.10 |
| 6-64 | 2.50 |
| 6-65 | 1.55 |
| 6-66 | 5.75 |
| 6-67 | 1.58 |
| 6-68 | 2.35 |
| 6-69 | 4.80 |
| 6-70 | 4.75 |
| 6-71 | 2.85 |
| 6-72 | 7.93 |
| 6-73 | 8.30 |
| 6-74 | 5.77 |
| 6-75 | 8.63 |
| 6-76 | 1.87 |
| 6-77 | 2.28 |
| 6-78 | 2.97 |
| 6-79 | 4.57 |
| 6-80 | 2.20 |
| 6-81 | 2.80 |
| 6-82 | 1.80 |
| 6-83 | 3.55 |
| 6-84 | 5.40 |
| 6-85 | 0.887 |
| 6-86 | 1.95 |
| 6-87 | 0.93 |
| 6-88 | 4.55 |
| 6-89 | 1.90 |
| 6-90 | 8.65 |
| 6-91 | 4.55 |
| 6-92 | 9.45 |
| 6-93 | 12.00 |
| 6-94 | 5.80 |
| 6-95 | 1.73 |
| 6-96 | 0.49 |
| 6-97 | 3.25 |
| 6-98 | 0.68 |
| 6-99 | 1.12 |
| 6-100 | 2.20 |
| 6-101 | 11.20 |
| 6-102 | 0.48 |
| 6-103 | 0.74 |
| 6-104 | 1.25 |
| 6-105 | 0.66 |
| 6-106 | 0.61 |
| 6-107 | 0.83 |
| 6-108 | 1.30 |
| 6-109 | 2.70 |
| 6-110 | 4.00 |
| 6-111 | 2.50 |
| 6-112 | 1.14 |
| 6-113 | 3.40 |
| 6-114 | 3.15 |
| 6-115 | 0.95 |
| 6-116 | 4.85 |
| 6-117 | 3.85 |
| 6-118 | 5.60 |
| 6-119 | 1.09 |
| 6-120 | 0.78 |
| 6-121 | 9.55 |
| 6-122 | 0.88 |
| 6-123 | 5.63 |
| 6-124 | 1.68 |
| 6-125 | 1.21 |
| 6-126 | 7.75 |
| 6-127 | 7.75 |
| 6-128 | 9.25 |
| 6-129 | 5.45 |
| 6-130 | 29.50 |
| 6-131 | 10.10 |
| 6-132 | 7.60 |
| 6-133 | 5.05 |
| 6-134 | 8.60 |
| 6-135 | 2.55 |
| 6-136 | 7.50 |
| 6-137 | 1.40 |
| 6-138 | 6.27 |
| 6-139 | 20.00 |
| 6-140 | 3.45 |
| 6-141 | 2.23 |
| 6-142 | 3.93 |
| 6-143 | 4.35 |
| 6-144 | 3.50 |
| 6-145 | 6.25 |
| 6-146 | 10.00 |
| 6-147 | 5.60 |
| 6-148 | 8.40 |
| 6-149 | 2.08 |
| 6-150 | 1.02 |
| 6-151 | 36.00 |
| 6-152 | 4.75 |
| 6-153 | 4.70 |
| 6-154 | 1.85 |
| 6-155 | 2.80 |
| 6-156 | 1.30 |
| 6-157 | 0.92 |
| 6-158 | 1.55 |
| 6-159 | 2.30 |
| 6-160 | 0.99 |
| 6-161 | 2.30 |
| 6-162 | 9.55 |
| 6-163 | 2.30 |
| 6-164 | 1.04 |
| 6-165 | 9.75 |
| 6-166 | 8.35 |
| 6-167 | 8.65 |
| 6-168 | |
| 6-169 | 4.05 |
| 6-170 | 2.10 |
| 6-171 | 2.30 |
| 6-172 | 1.70 |
| 6-173 | 2.45 |
| 6-174 | 1.02 |
| 6-175 | 4.85 |
| 6-176 | 4.00 |
| 6-177 | 0.67 |
| 6-178 | 2.15 |
| 6-179 | 2.00 |
| 6-180 | 0.84 |
| 6-181 | 5.55 |
| 6-182 | 9.00 |
| 6-183 | 3.00 |
| 6-184 | 3.65 |
| 6-185 | 2.35 |
| 6-186 | 5.35 |
| 6-187 | 1.45 |
| 6-188 | 2.35 |
| 6-189 | 1.35 |
| 6-190 | 2.25 |
| 6-191 | 0.97 |
| 6-192 | 3.03 |
| 6-193 | 4.05 |
| 6-194 | 2.55 |
| 6-195 | 1.55 |
| 6-196 | 0.29 |
| 6-197 | 3.65 |
| 6-198 | |
| 6-199 | 10.50 |
| 6-200 | 2.30 |
| 6-201 | 3.05 |
| 6-202 | 0.97 |
| 6-203 | 1.50 |
| 6-204 | 8.15 |
| 6-205 | 8.55 |
| 6-206 | 3.10 |
| 6-207 | 4.35 |
| 6-208 | 5.10 |
| 6-209 | 1.13 |
| 6-210 | 1.45 |
| 6-211 | 1.18 |
| 6-212 | 8.15 |
| 6-213 | 3.15 |
| 6-214 | 1.35 |
| 6-215 | 2.70 |
| 6-216 | 5.20 |
| 6-217 | 1.95 |
| 6-218 | 1.95 |
| 6-219 | 1.15 |
| 6-220 | 1.18 |
| 6-221 | 0.73 |
| 6-222 | 1.11 |
| 6-223 | 1.25 |
| 6-224 | 2.00 |
| 6-225 | 7.40 |
| 6-226 | 0.70 |
| 6-227 | 6.67 |
| 6-228 | 1.10 |
| 6-229 | 3.37 |
| 6-230 | 1.60 |
| 6-231 | 3.25 |
| 6-232 | 2.05 |
| 6-233 | 0.37 |
| 6-234 | 8.67 |
| 6-235 | |
| 6-236 | 8.75 |
| 6-237 | 1.90 |
| 6-238 | 1.40 |
| 6-239 | 3.10 |
| 6-240 | |
| 6-241 | 9.50 |
| 6-242 | |
| 6-243 | 2.00 |
| 6-244 | 5.55 |
| 6-245 | 1.35 |
| 6-246 | 5.80 |
| 6-247 | 6.00 |
| 6-248 | 2.45 |
| 6-249 | 3.00 |
| 6-250 | 6.30 |
| 6-251 | 1.07 |
| 6-252 | 0.84 |
| 6-253 | 4.75 |
| 6-254 | 2.30 |
| 6-255 | 0.95 |
| 6-256 | 1.10 |
| 6-257 | 3.30 |
| 6-258 | 0.82 |
| 6-259 | 0.64 |
| 6-260 | 0.82 |
| 6-261 | 2.70 |
| 6-262 | 2.80 |
| 6-263 | 2.25 |
| 6-264 | 2.25 |
| 6-265 | 1.07 |
| 6-266 | 2.85 |
| 6-267 | 0.32 |
| 6-268 | 8.80 |
| 6-269 | 1.75 |
| 6-270 | 2.67 |
| 6-271 | 0.87 |
| 6-272 | 1.22 |
| 6-273 | 1.10 |
| 6-274 | 1.30 |
| 6-275 | 2.85 |
| 6-276 | 0.91 |
| 6-277 | 4.10 |
| 6-278 | 4.50 |
| 6-279 | 0.49 |
| 6-280 | 1.50 |
| 6-281 | 2.05 |
| 6-282 | 1.60 |
| 6-283 | 0.41 |
| 6-284 | 1.80 |
| 6-285 | 3.35 |
| 6-286 | 2.65 |
| 6-287 | 1.35 |
| 6-288 | 0.78 |
| 6-289 | 1.20 |
| 6-290 | 3.40 |
| 6-291 | 5.60 |
| 6-292 | 2.05 |
| 6-293 | 5.70 |
| 6-294 | 9.95 |
| 6-295 | 2.30 |
| 6-296 | 1.60 |
| 6-297 | 1.15 |
| 6-298 | 2.85 |
| 6-299 | 4.40 |
| 6-300 | 3.30 |
| 6-301 | 1.40 |
| 6-302 | 2.95 |
| 6-303 | 2.90 |
| 6-304 | 0.65 |
| 6-305 | 2.95 |
| 6-306 | 1.37 |
| 6-307 | 1.65 |
| 6-308 | 2.55 |
| 6-309 | 1.13 |
| 6-310 | 0.91 |
| 6-311 | 1.05 |
| 6-312 | 1.30 |
| 6-313 | 1.65 |
| 6-314 | 0.85 |
| 6-315 | 2.00 |
| 6-316 | 0.74 |
| 6-317 | 0.93 |
| 6-318 | 3.20 |
| 6-319 | 1.17 |
| 6-320 | 1.35 |
| 6-321 | 1.70 |
| 6-322 | 3.03 |
| 6-323 | 3.30 |
| 6-324 | 0.97 |
| 6-325 | 0.65 |
| 6-326 | 2.70 |
| 6-327 | 0.48 |
| 6-328 | 9.30 |
| 6-329 | 5.45 |
| 6-330 | 9.30 |
| 6-331 | 4.85 |
| 6-332 | 2.00 |
| 6-333 | 4.00 |
| 6-334 | 4.45 |
| 6-335 | 1.80 |
| 6-336 | 3.45 |
| 6-337 | 1.50 |
| 6-338 | 0.94 |
| 6-339 | 7.15 |
| 6-340 | 1.02 |
| 6-341 | 0.99 |
| 6-342 | 1.07 |
| 6-343 | 0.28 |
| 6-344 | 0.99 |
| 6-345 | 1.55 |
| 6-346 | 8.30 |
| 6-347 | 3.50 |
| 6-348 | 3.55 |
| 6-349 | 0.88 |
| 6-350 | 0.78 |
| 6-351 | 7.00 |
| 6-352 | 2.50 |
| 6-353 | 3.73 |
| 6-354 | 4.50 |
| 6-355 | 5.80 |
| 6-356 | 4.35 |
| 6-357 | 1.95 |
| 6-358 | 2.45 |
| 6-359 | 1.60 |
| 6-360 | 6.13 |
| 6-361 | 6.90 |
| 6-362 | 27.00 |
| 6-363 | 46.00 |
| 6-364 | 6.45 |
| 6-365 | 3.15 |
| 6-366 | 2.20 |
| 6-367 | 0.69 |
| 6-368 | 0.87 |
| 6-369 | 3.05 |
| 6-370 | |
| 6-371 | 2.70 |
| 6-372 | 0.90 |
| 6-373 | 2.10 |
| 6-374 | 1.00 |
| 6-375 | 1.07 |
| 6-376 | 5.07 |
| 6-377 | 1.60 |
| 6-378 | 2.10 |
| 6-379 | 1.80 |
| 6-380 | 1.30 |
| 6-381 | 1.52 |
| 6-382 | 0.78 |
| 6-383 | 1.60 |
| 6-384 | 3.40 |
| 6-385 | 1.30 |
| 6-386 | 3.10 |
| 6-387 | 2.85 |
| 6-388 | 6.05 |
| 6-389 | 3.10 |
| 6-390 | 8.50 |
| 6-391 | 3.30 |
| 6-392 | 4.40 |
| 6-393 | 0.85 |
| 6-394 | 6.65 |
| 6-395 | 6.60 |
| 6-396 | 3.05 |
| 6-397 | 3.45 |
| 6-398 | 3.45 |
| 6-399 | 9.10 |
| 6-400 | 3.65 |
| 6-401 | 3.20 |
| 6-402 | 0.82 |
| 6-403 | 0.69 |
| 6-404 | 5.95 |
| 6-405 | 4.70 |
| 6-406 | 23.50 |
| 6-407 | 1.03 |
| 6-408 | 1.03 |
| 6-409 | 7.12 |
| 6-410 | 5.18 |
| 6-411 | 32.50 |
| 6-412 | 1.43 |
| 6-413 | 3.43 |
| 6-414 | 5.55 |
| 6-415 | 1.05 |
| 6-416 | 2.80 |
| 6-417 | 1.85 |
| 6-418 | 2.18 |
| 6-419 | 1.50 |
| 6-420 | |
| 6-421 | 1.49 |
| 6-422 | 0.70 |
| 6-423 | 0.62 |
| 6-424 | 0.68 |
| 6-425 | 0.52 |
| 6-426 | 0.82 |
| 6-427 | 5.15 |
| 6-428 | 0.81 |
| 6-429 | 1.09 |
| 6-430 | 1.90 |
| 6-431 | 0.49 |
| 6-432 | 1.10 |
| 6-433 | 0.76 |
| 6-434 | 1.95 |
| 6-435 | 0.26 |
| 6-436 | 0.84 |
| 6-437 | 0.77 |
| 6-438 | 0.55 |
| 6-439 | 1.00 |
| 6-440 | 1.05 |
| 6-441 | 1.45 |
| 6-442 | 5.25 |
| 6-443 | 2.45 |
| 6-444 | 3.23 |
| 6-445 | 7.10 |
| 6-446 | 8.05 |
| 6-447 | 4.55 |
| 6-448 | 3.05 |
| 6-449 | 2.30 |
| 6-450 | 12.00 |
| 6-451 | 5.35 |
| 6-452 | 6.20 |
| 6-453 | 1.13 |
| 6-454 | 4.75 |
| 6-455 | 2.85 |
| 6-456 | 5.95 |
| 6-457 | 4.65 |
| 6-458 | 2.15 |
| 6-459 | 5.60 |
| 6-460 | 1.70 |
| 6-461 | 1.65 |
| 6-462 | 1.20 |
| 6-463 | 0.75 |
| 6-464 | 3.60 |
| 6-465 | 1.75 |
| 6-466 | 5.40 |
| 6-467 | 2.35 |
| 6-468 | 4.45 |
| 6-469 | 3.35 |
| 6-470 | 3.55 |
| 6-471 | 1.80 |
| 6-472 | 1.16 |
| 6-473 | 3.30 |
| 6-474 | 1.63 |
| 6-475 | 2.58 |
| 6-476 | 5.30 |
| 6-477 | 0.44 |
| 6-478 | 1.20 |
| 6-479 | 2.30 |
| 6-480 | 3.05 |
| 6-481 | 0.57 |
| 6-482 | 0.48 |
| 6-483 | 1.01 |
| 6-484 | 1.80 |
| 6-485 | 3.40 |
| 6-486 | 2.10 |
| 6-487 | 0.67 |
| 6-488 | 0.57 |
| 6-489 | 0.41 |
| 6-490 | 0.43 |
| 6-491 | 0.58 |
| 6-492 | 1.80 |
| 6-493 | 2.98 |
| 6-494 | 5.40 |
| 6-495 | 0.61 |
| 6-496 | 1.20 |
| 6-497 | 4.37 |
| 6-498 | 7.13 |
| 6-499 | 13.00 |
| 6-500 | 6.75 |
| 6-501 | 5.70 |
| 6-502 | 2.75 |
| 6-503 | 4.70 |
| 6-504 | 1.35 |
| 6-505 | 1.03 |
| 6-506 | 5.45 |
| 6-507 | 2.55 |
| 6-508 | 6.15 |
| 6-509 | 1.08 |
| 6-510 | 8.53 |
| 6-511 | 0.59 |
| 6-512 | 0.95 |
| 6-513 | 7.10 |
| 6-514 | 9.25 |
| 6-515 | 3.00 |
| 6-516 | 4.40 |
| 6-517 | 4.20 |
| 6-518 | 1.70 |
| 6-519 | 6.90 |
| 6-520 | 3.15 |
| 6-521 | 2.23 |
| 6-522 | 16.90 |
| 6-523 | 10.90 |
| 6-524 | 7.00 |
| 6-525 | 7.80 |
| 6-526 | 9.43 |
| 6-527 | 3.37 |
| 6-528 | 4.25 |
| 6-529 | 1.11 |
| 6-530 | 0.81 |
| 6-531 | 5.50 |
| 6-532 | 8.90 |
| 6-533 | 5.55 |
| 6-534 | 8.20 |
| 6-535 | 4.20 |
| 6-536 | 3.80 |
| 6-537 | 1.63 |
| 6-538 | 2.77 |
| 6-539 | 3.30 |
| 6-540 | 0.85 |
| 6-541 | 0.97 |
| 6-542 | 10.50 |
| 6-543 | 3.30 |
| 6-544 | 1.65 |
| 6-545 | 0.77 |
| 6-546 | 3.13 |
| 6-547 | 7.80 |
| 6-548 | 8.10 |
| 6-549 | 1.10 |
| 6-550 | 0.84 |
| 6-551 | 1.70 |
| 6-552 | 2.55 |
| 6-553 | 2.80 |
| 6-554 | 7.25 |
| 6-555 | 3.35 |
| 6-556 | 2.15 |
| 6-557 | 4.30 |
| 6-558 | 1.15 |
| 6-559 | 6.07 |
| 6-560 | 1.60 |
| 6-561 | 1.10 |
| 6-562 | 1.85 |
| 6-563 | 2.90 |
| 6-564 | 4.05 |
| 6-565 | 2.05 |
| 6-566 | 2.25 |
| 6-567 | 2.50 |
| 6-568 | 1.05 |
| 6-569 | 1.45 |
| 6-570 | 1.30 |
| 6-571 | 1.50 |
| 6-572 | 2.80 |
| 6-573 | 1.25 |
| 6-574 | 1.50 |
| 6-575 | 1.95 |
| 6-576 | 3.75 |
| 6-577 | 2.70 |
| 6-578 | 2.30 |
| 6-579 | 0.86 |
| 6-580 | 1.25 |
| 6-581 | 1.60 |
| 6-582 | 2.65 |
| 6-583 | 0.63 |
| 6-584 | 7.40 |
| 6-585 | 3.00 |
| 6-586 | 1.35 |
| 6-587 | 0.83 |
| 6-588 | 5.60 |
| 6-589 | 2.55 |
| 6-590 | 1.20 |
| 6-591 | 1.30 |
| 6-592 | 1.85 |
| 6-593 | 8.43 |
| 6-594 | 0.46 |
| 6-595 | 3.10 |
| 6-596 | 2.80 |
| 6-597 | 1.15 |
| 6-598 | 2.35 |
| 6-599 | 2.10 |
| 6-600 | 2.30 |
| 6-601 | 4.25 |
| 6-602 | 5.65 |
| 6-603 | 9.33 |
| 6-604 | 1.85 |
| 6-605 | 3.80 |
| 6-606 | 2.30 |
| 6-607 | 2.95 |
| 6-608 | 1.90 |
| 6-609 | 1.50 |
| 6-610 | 6.35 |
| 6-611 | 2.73 |
| 6-612 | 9.30 |
| 6-613 | 3.67 |
| 6-614 | 0.82 |
| 6-615 | 0.69 |
| 6-616 | 0.78 |
| 6-617 | 7.65 |
| 6-618 | 1.00 |
| 6-619 | 1.15 |
| 6-620 | 1.50 |
| 6-621 | 1.15 |
| 6-622 | 1.25 |
| 6-623 | 3.15 |
| 6-624 | 6.70 |
| 6-625 | 9.95 |
| 6-626 | 1.02 |
| 6-627 | 0.58 |
| 6-628 | 6.65 |
| 6-629 | 3.10 |
| 6-630 | 5.10 |
| 6-631 | 8.55 |
| 6-632 | 6.30 |
| 6-633 | 3.50 |
| 6-634 | 3.70 |
| 6-635 | 4.05 |
| 6-636 | 7.63 |
| 6-637 | 3.60 |
| 6-638 | 1.36 |
| 6-639 | 0.74 |
| 6-640 | 9.40 |
| 6-641 | 7.50 |
| 6-642 | 1.15 |
| 6-643 | 1.95 |
| 6-644 | 6.55 |
| 6-645 | 6.00 |
| 6-646 | 5.40 |
| 6-647 | 3.03 |
| 6-648 | 2.77 |
| 6-649 | 9.97 |
| 6-650 | 1.55 |
| 6-651 | 1.00 |
| 6-652 | 10.00 |
| 6-653 | 27.50 |
| 6-653 | 12.80 |
| 6-655 | 9.35 |
| 6-656 | 1.40 |
| 6-657 | 1.40 |
| 6-658 | 7.00 |
| 6-659 | 8.55 |
| 6-660 | 3.00 |
| 6-661 | 1.43 |
| 6-662 | 6.10 |
| 6-663 | 3.85 |
| 6-664 | 5.25 |
| 6-665 | 3.10 |
| 6-666 | 1.40 |
| 6-667 | 6.00 |
| 6-668 | 1.09 |
| 6-669 | 4.00 |
| 6-670 | 2.00 |
| 6-671 | 1.55 |
| 6-672 | 1.70 |
| 6-673 | 3.30 |
| 6-674 | 0.79 |
| 6-675 | 1.50 |
| 6-676 | 3.55 |
| 6-677 | 0.45 |
| 6-678 | 5.20 |
| 6-679 | 1.09 |
| 6-680 | 19.0 |
| 6-681 | 5.25 |
| 6-682 | 1.09 |
| 6-683 | 1.06 |
| 6-684 | 3.35 |

B-2. Inhibition of Human Platelet Aggregation In Vitro

Blood Collection

Human blood is collected by antecubital venipuncture using a 20G multifly set (Sarstedt, Numbrecht, Germany) into plastic tubes (Sarstedt 9 NC/10 ml monovettes) containing 3.8% (w/v) sodium citrate (1/10 v/v) from healthy donors who denied having taken any medication for at least 7 days prior to the study. Tubes are gently mixed to prevent coagulation.

Preparation of Platelet Rich Plasma (PRP) and Platelet Poor Plasma (PPP)

Blood is centrifuged at 140 g for 20 min at room temperature and the supernatant PRP is immediately collected into Falcon tubes. PRP is further centrifuged (11.000 rpm, 1 min) to produce platelet poor plasma (PPP).

Measurement of Platelet Aggregation

Platelet aggregation is analyzed by light transmittance aggregometry according to the method of Born (J. Physiol. 1963, 168, 178-195) using APACT aggregometers (Rolf Greiner BioChemicals, Flacht, Germany).

178 μl of PRP is incubated with test compound in various concentrations dissolved in 2 μl DMSO in the aggregation cuvette at 37° C. for 5 min. Then 2 μl sulprostone is added and is incubated at 37° C. for 2 min. Aggregation is started by addition of 20 μl of collagen. The following concentrations of agonists are used: sulprostone 0.02 μM-2.5μM (Sigma-Aldrich), collagen 0.05-0.6 μg/ml (Horm®, Nycomed).

The reaction is followed by monitoring changes in light transmission on PRP aliquots against PPP control. Light transmittance is recorded for 5 minutes and the maximal aggregation value is used for IC₅₀ calculations.

Statistical Analysis

IC₅₀ is calculated with GrapPad Prism (sigmoidal dose-response). All values are expressed as means±SEM, with n denoting the number of blood donors.

B-3. EP3 Binding Assay to Identify Inhibitors of PGE2 Binding Using Scintillation Proximity Assay Technique

To compound of interest (in DMSO, 2 μl, typical final concentration up to 20 μM) the following reagents are added in a total volume of 152 μl in reaction buffer (50 mM Tris/HCl, pH 7.5; 10 mM MgCl2; 1 mM EDTA) in a suitable 96 well clear bottom plate:

[3H] Prostaglandin E2 ([5,6,8,11,12,14,15-3H(N)] in a final concentration of 1.5 nM;

EP3 Prostanoid Receptor Membrane Preparation (2 mg/ml) and

PVT-WGA Beads in a final concentration of 2.5 mg/ml.

The mixture is incubated for 2 h at room temperature. Scintillation is monitored in a suitable microplate reader equipped with 3H-SPA program. The values are used to plot dose response curves and calculate EC₅₀ values for the test compounds.

B-4. Determination of the Effect on Laser-Induced Thrombus Formation in Mice (In Vivo)

This study serves to investigate the efficacy of a test substance on thrombus formation, stability and resolution induced by laser injury to microvessels of atherosclerotic mice.

Animals

Male ApoE−/−mice (Charles River) are fed an atherogenic diet (Ssniff S0602-S170, 20.85% fat, 0.15% cholesterol, 19.5% casein) from 6 weeks of age for at least 3 months. Test compound treatment is performed either orally via gavage to conscious animals before anesthesia or intravenously to anesthetized animals prior to injury.

Thrombus Induction

For oral treatment, animals are dosed by gavage in appropriate formulations by gavage prior to anesthesia. For intravenous treatment, a venous catheter will be implanted in the jugular vein for bolus or infusion drug delivery prior to the injury procedure. Mice are anesthetized by intraperitoneal injection of Ketamin (65 mg/kg) and Xylazin (15 mg/kg) The cremaster muscle is exposed after opening the scrotum and removal of a testis with attention to preserve blood supply from the circulation of the mouse. A stainless steel wire loop (Unimed 30.065, diameter 0.4 mm) is used to spread the inverted cremaster pocket in an organ bath constantly perfused with Dulbecco's phosphate buffered saline temperated at 37° C. The tissue preparation is positioned on top of a transparent macrolon plastic stage for intravital microscopy (Leica AS LMD, water immersion objective Leica 506155 HC APO/L40×/0.80). Video images are acquired by a digital camera (Basler PCO.EDGE 5.5) for identification of arterioles suitable for vascular wall injury (50-100 μm diameter, bifurcation-free segment).

A microdissection UV-Laser (wavelength 355 nm, Cell Surgeon, LLS Rowiak, Hannover, Germany) coupled into the optical path of the intravital microscope is used for 2-stage injury of the vessel wall: (1) Linear detachment of the endothelium along a length of approximately 150 μm. (2) Punctiform deep injury with local damage of the media. The resulting thrombus formation is to visualized and recorded over a duration of 5 minutes. The lesion procedure will be repeated on an upstream segment of the identical vessel for at least two times.

Statistical Analysis

Video sequences of thrombus development are quantified by morphometric analysis of images at 10-second intervals using a customized software for semi-automatic vessel and thrombus detection (Zeta, Fraunhofer-Institut fur Angewandte Informationstechnik FIT, St. Augustin, Germany). Thrombus area and degree of stenosis are calculated to generate a thrombus-over-time-curve. The main derived result is the time above 75% occlusion of the vessel lumen. The mean of at least three repeated injuries per animal will be calculated. In case of intravenous testing, the treatment effect will be compared to baseline control injuries in the identical animals (t-test for repeated measurements). In case of oral treatment, thrombus development will be compared to vehicle-treated animals (ANOVA and t-test for independent measurements). A minimum of 5 animals will be tested to obtain sufficient statistical power.

LENGTHY TABLES
The patent application contains a lengthy table section. A copy of the table is available in electronic form from the USPTO web site (https://seqdata.uspto.gov/?pageRequest=docDetail&DocID=US20220324865A1). An electronic copy of the table will also be available from the USPTO upon request and payment of the fee set forth in 37 CFR 1.19(b)(3).

1. A compound of formula (I) 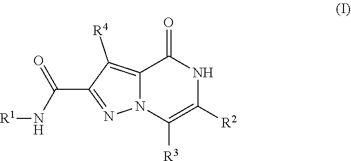
wherein
R¹ represents C₁-C₆-alkyl, C₂-C₆-halogenoalkyl, C₃-C₆-cycloalkyl or the group -L-R^{E},
where alkyl may be substituted by 1 or 2 substituents independently selected from the group consisting of hydroxyl, cyano, cyclopropyl, cyclobutyl, azetidin-1-yl (which may be substituted by 1 or 2 fluorine atoms), methoxy, methylsulfonyl, carbamoyl, NR^{a}R^{b} (where R^{a} and R^{b} are independently selected from the group consisting of hydrogen, C₁-C₄-alkyl, C₂-C₆-halogenoalkyl or cyclopropyl, or where R^{a} and R^{b} together with the nitrogen atom to which they are bound may form a morpholine ring) and C₁-C₃-halogenoalkoxy, where halogenoalkoxy is substituted by 1 to 3 fluorine atoms,
and
where halogenoalkyl is substituted by 1 to 6 fluorine atoms and may be further substituted by 1 or 2 substituents independently selected from the group consisting of hydroxyl, methoxycarbonyl, NR^{c}R^{d} (where R^{c} and R^{d} are independently selected from the group consisting of hydrogen, C₁-C₄-alkyl, C₂-C₆-halogenoalkyl or cyclopropyl, or where R^{c} and R^{d} together with the nitrogen atom to which they are bound may form a morpholine ring),
and
where in the cycloalkyl ring one CH₂ group may be replaced by CR^{e}R^{f}, O, SO₂ or NR⁵, and where the cycloalkyl may be substituted by 1 substituent selected from the group consisting of methyl, ethyl, n-propyl, isopropyl, hydroxyl, trifluoromethyl, di-(C₁-C₂-alkyl)amino-methyl, cyano, phenyl and pyridinyl, or may be substituted by 1 or 2 fluorine substituents,
where the phenyl may be substituted by 1 or 2 substituents independently selected from the group consisting of fluorine, chlorine, methoxy and cyano,
and
where the pyridinyl may be substituted by 1 methoxy substituent,
and
R^{e} and R^{f} together with the carbon atom to which they are bound form another C₃-C₆-cycloalkyl, where again one CH₂ group may be replaced by SO₂,
and
R⁵ represents hydrogen, C₁-C₄-alkyl, C₁-C₂-halogenoalkyl substituted by 1 to 3 fluorine atoms, cyclopropyl, methylcarbonyl, methoxycarbonyl or tert-butoxycarbonyl,
and
L represents a bond or Ci-C₆-alkanediyl,
where alkanediyl may be substituted by 1 or 2 substituents independently selected from the group consisting of chlorine, hydroxyl, methoxy, methoxycarbonyl, carboxyl, carbamoyl, cyclopropyl, 1-hydroxycyclopropyl, amino, dimethylamino, (ethyl)(2-hydroxyethyl)amino, tert-butoxycarbonylamino, N₃, azetidin-1-yl (which may be substituted by 1 or 2 fluorine atoms), pyrrolidin-1-yl (which may be substituted by 1 or 2 fluorine atoms), morpholin-4-yl, 1H-1,2,4-triazol-1-yl and N-tert-butoxy-azeditin-3-yl and additionally by up to 3 fluorine atoms,
R^{E} represents phenyl, phenoxy, pyridinyl, pyrimidinyl, pyrazinyl, thienyl, pyrazolyl, oxazolyl, isoxazolyl, imidazolyl, 1,2,4-oxadiazolyl, 1,2,4-triazolyl, naphthyl, 1,2,3,4-tetrahydronaphthalen-1-yl, quinolinyl, benzimidazolyl, 2-oxo-2,3-dihydro-1H-benzimidazol-5-yl, indolyl, 2,3-dihydro-1-H-indenyl, benzodioxolyl, 2,3-dihydro-benzodioxinyl, 3,4-dihydro-2H-chromen-4-yl, cyclohexyl, morpholin-4-yl, azetidin-1-yl, pyrrolidin-1-yl, 2-oxo-1,3-oxazolidin-5-yl or 4-cyclopropyl-2,5-dioxoimidazolidin-4-yl,
where phenyl may be substituted by 1 or 2 substituents independently selected from the group consisting of halogen, C₁-C₄-alkyl, hydroxyl, methoxy, trifluoromethyl, trifluoromethoxy, difluoromethoxy, dimethylaminomethyl, methylsulfonyl, sulfamoyl and pyrrolidin-1-ylmethyl,
where phenoxy may be substituted by 1 or 2 substituents independently selected from the group consisting of chlorine and methyl,
where pyridinyl may be substituted by 1 or 2 substituents independently selected from the group consisting of chlorine, methyl, trifluoromethyl, methoxy and 2,2,2-trifluoroethoxy,
where pyrimidinyl may be substituted by 1 or 2 methyl substituents,
where pyrazinyl may be substituted by one 2,2,2-trifluoroethoxy substituent,
where pyrazolyl may be substituted by 1 to 3 substituents independently selected from the group consisting of chlorine, C₁-C₄-alkyl and cyclopropyl,
where oxazolyl may be substituted by 1 methyl substituent,
where imidazolyl may be substituted by 1 methyl substituent,
where 1,2,4-oxadiazol may be substituted by 1 methyl substituent,
where 1,2,3,4-tetrahydronaphthalen-1-yl may be substituted by 1 methoxy substituent,
where 1,2,4-triazolyl may be substituted by 1 methyl or ethyl substituent,
where indolyl may be substituted by 1 or 2 methyl substituents,
where 2,3-dihydro-1-H-indenyl may be substituted by 1 hydroxyl substituent,
where 3,4-dihydro-2H-chromen-4-yl may be disubstituted in 2-position by methyl and additionally substituted by 1 substituent selected from methyl and methoxy,
where azetidin-1-yl may be substituted by 1 fluorine or hydroxyl substituent,
where pyrrolidin-1-yl may be substituted by 1 fluorine or hydroxyl substituent,
R² represents a group of formula 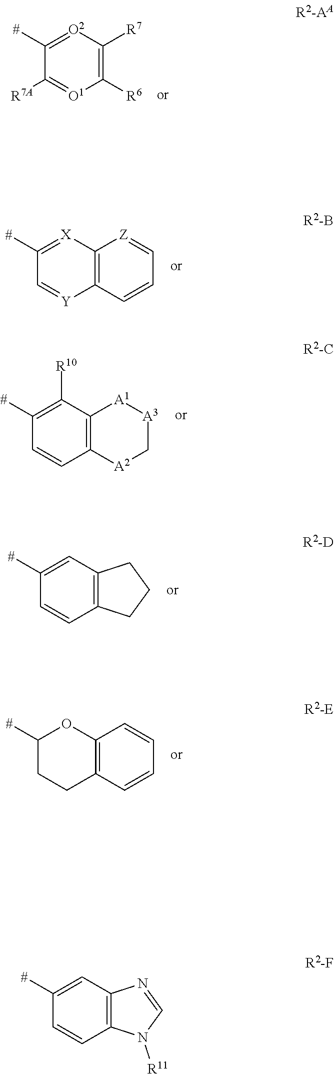
where # is the point of attachment to the pyrazinone ring,
Q¹ represents CR^{8A} or N,
Q² represents CR⁸ or N,
R⁶ represents hydrogen, halogen, C₁-C₄-alkyl, C₁-C₄-halogenoalkyl, C₁-C₄-alkoxy, C₁-C₄-halogenoalkoxy or C₃-C₆-cycloalkyl,
R⁷ represents hydrogen, halogen, C₁-C₄-alkyl, C₁-C₄-halogenoalkyl, C₁-C₄-alkoxy, C₁-C₄-halogenoalkoxy or C₃-C₆-cycloalkyl,
with the proviso, that at least one or R⁶ and R⁷ is not hydrogen,
R^{7A} represents hydrogen or halogen,
R⁸ represents hydrogen or halogen,
R^{8A} represents hydrogen or halogen,
X represents CH or N,
Y represents CH or N,
Z represents CR⁹ or N,
where at most one of X, Y and Z is N,
R⁹ represents hydrogen, halogen or C₁-C₄-alkyl,
A¹ represents CH₂, O or NMe,
A² represents CH₂, O or NMe,
A³ represents CH₂ or O,
where both A¹ and A² are different from O, if A³ is O,
R¹⁰ represents hydrogen or halogen,
R¹¹ represents hydrogen or C₁-C₄-alkyl,
R³ represents hydrogen, halogen or C₁-C₄-alkyl,
R⁴ represents hydrogen, halogen, C₁-C₄-alkyl, C₁-C₄-halogenoalkyl, C₃-C₆-cycloalkyl, cyano or C₁-C₃-alkoxymethyl,
where in the cycloalkyl ring one carbon may be replaced by NR¹², and where the cycloalkyl may be substituted by 1 or 2 fluorine atoms,
R12 represents hydrogen, C₁-C₄-alkyl or C₁-C₄-alkylaminocarbonyl,
and/or a salt thereof, N-oxide, solvate thereof and/or solvate of a salt and/or of an N-oxide thereof.

2. The compound according to claim 1, wherein
R¹ represents C₁-C₅-alkyl, C₂-C₄-halogenoalkyl, C₃-C₄-cycloalkyl or the group -L-R^{E},
where alkyl may be substituted by 1 or 2 substituents independently selected from the group consisting of hydroxyl, cyano, cyclopropyl, 3-fluoroazetidin-1-yl, 3,3-difluoroazetidin-1-yl, NR^{a}R^{b} (where R^{a} and R^{b} are independently selected from the group consisting of hydrogen, methyl, 2,2-difluoroethyl, 2,2,2-trifluoroethyl or cyclopropyl, or where R^{a} and Rb together with the nitrogen atom to which they are bound may form a morpholine ring) and C₁-C₂-halogenoalkoxy, where halogenoalkoxy is substituted by 1 to 3 fluorine atoms,
and
where halogenoalkyl is substituted by 1 to 6 fluorine atoms and may be further substituted by 1 or 2 hydroxyl substituents,
and
where in the cycloalkyl ring one CH2 group may be replaced by NR⁵, and where the cycloalkyl may be substituted by 1 substituent selected from the group consisting of methyl, trifluoromethyl, diethylamino-methyl, phenyl and pyridin-3-yl,
where the phenyl may be substituted by 1 or 2 substituents independently selected from the group consisting of fluorine, chlorine and methoxy,
and
where the pyridinyl may be substituted by 1 methoxy substituent,
and
R⁵ represents hydrogen, methyl, ethyl, n-propyl, 2-methyl-propan-1-yl, 2,2-difluoroethyl, 2,2,2-trifluoroethyl, 1,3-difluoropropan-2-yl or cyclopropyl,
and
L represents a bond or C₁-C₅-alkanediyl,
where alkanediyl may be substituted by 1 or 2 substituents independently selected from the group consisting of hydroxyl, cyclopropyl, 1-hydroxycyclopropyl, amino, dimethylamino, (ethyl)(2-hydroxyethyl)amino, azetidin-1-yl, 3-fluoroazetidin-1-yl, 3-fluoroazetidin-1-yl, pyrrolidin-1-yl, 3-fluoropyrrolidin-1-yl, 3,3-difluoropyrrolidin-1-yl, morpholin-4-yl, and additionally by up to 3 fluorine atoms,
R^{E} represents phenyl, pyridin-3-yl, pyridin-2-yl, pyrazol-4-yl, pyrazol-1-yl, pyrazol-5-yl or pyrazol-3-yl,
where phenyl may be substituted by 1 or 2 substituents independently selected from the group consisting of fluorine, chlorine, bromine, methyl, ethyl, hydroxyl, methoxy, trifluoromethyl, trifluoromethoxy and difluoromethoxy,
where pyridinyl may be substituted by 1 or 2 substituents independently selected from the group consisting of chlorine, methyl, trifluoromethyl, methoxy and 2,2,2-trifluoroethoxy,
where pyrazolyl may be substituted by 1 to 3 substituents independently selected from the group consisting of chlorine, methyl, ethyl, isopropyl and cyclopropyl,
R² represents a group of formula 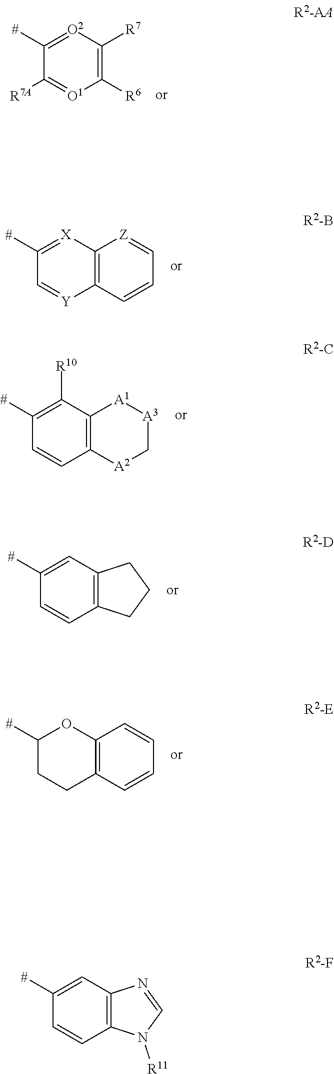
where # is the point of attachment to the pyrazinone ring,
Q¹ represents CR^{8A} or N,
Q² represents CR⁸ or N,
R⁶ represents hydrogen, fluorine, chlorine, methyl, ethyl, difluoromethyl, trifluoromethyl, methoxy, trifluoromethoxy or cyclopropyl,
R⁷ represents hydrogen, fluorine, chlorine, methyl, ethyl, difluoromethyl, trifluoromethyl, methoxy, trifluoromethoxy or cyclopropyl,
with the proviso, that at least one or R⁶ and R⁷ is not hydrogen,
R^{7A} represents hydrogen, fluorine or chlorine.
R⁸ represents hydrogen, fluorine or chlorine,
R^{8A} represents hydrogen, fluorine or chlorine,
X represents CH or N,
Y represents CH or N,
Z represents CR⁹ or N,
where at most one of X, Y and Z is N,
R⁹ represents hydrogen, fluorine, chlorine, methyl or ethyl,
A¹ represents CH₂, O or NMe,
A² represents CH₂, O or NMe,
A³ represents CH₂ or O,
where both A¹ and A² are different from O, if A³ is O,
R¹⁰ represents hydrogen or fluorine,
R¹¹ represents methyl,
R³ represents hydrogen, fluorine, chlorine, cyano, methyl or ethyl,
R⁴ represents hydrogen, fluorine, chlorine, methyl, ethyl, n-propyl, isopropyl, difluoromethyl, trifluoromethyl, pentafluoroethyl, cyclopropyl, 2,2-difluorocyclopropan-1-yl, cyclobutyl, 3,3-difluorocyclobutan-1-yl, piperidin-4-yl, cyano or methoxymethyl,
where piperidin-4-yl may be substituted in 1-position by methyl, methylaminocarbonyl or ethylaminocarbonyl,
and/or a salt thereof, N-oxide, solvate thereof and/or solvate of a salt and/or of an N-oxide thereof.

3. The compound according to claim 1, wherein
R¹ represents ethyl, propan-1-yl, propan-2-yl, butan-2-yl, C₂-C₄-halogenoalkyl, cyclopropyl, cyclobutyl or the group -L-R^{E},
where ethyl, the propanyls and butanyl may be substituted by 1 substituent selected from the group consisting of hydroxyl and cyclopropyl,
and
where halogenoalkyl is substituted by 1 to 3 fluorine atoms and may be further substituted by 1 hydroxyl substituent,
and
where in the cyclobutyl ring one carbon may be replaced by NR⁵, and where the cyclopropyl or cyclobutyl may be substituted by 1 substituent selected from the group consisting of methyl and phenyl,
where the phenyl may be substituted by 1 or 2 substituents independently selected from the group consisting of fluorine and methoxy,
and
R⁵ represents hydrogen, methyl, ethyl, n-propyl, 2-methyl-propan-1-yl, 2,2-difluoroethyl, 2,2,2-trifluoroethyl, 1,3-difluoropropan-2-yl or cyclopropyl,
and
L represents C₁-C₄-alkanediyl,
where alkanediyl may be substituted by 1 substituent selected from the group consisting of hydroxyl, 1-hydroxycyclopropyl, dimethylamino, and additionally by up to 3 fluorine atoms,
R^{E} represents phenyl, pyridin-3-yl or pyridin-2-yl,
where phenyl may be substituted by 1 or 2 substituents independently selected from the group consisting of fluorine, chlorine, methyl, hydroxyl, methoxy, trifluoromethyl and difluoromethoxy,
where pyridinyl may be substituted by 1 or 2 substituents independently selected from the group consisting of chlorine, trifluoromethyl, trifluoromethoxy and 2,2,2-trifluoroethoxy,
R² represents a group of formula 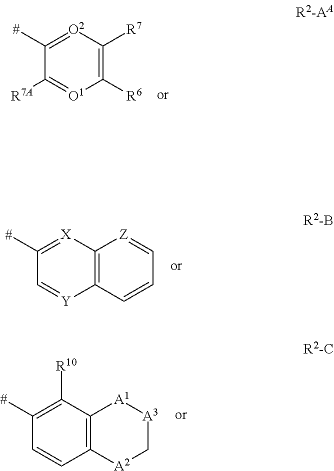
where # is the point of attachment to the pyrazinone ring,
Q¹ represents CR^{8A},
Q² represents CR⁸,
R⁶ represents hydrogen, fluorine, chlorine, methyl, difluoromethyl, trifluoromethyl, methoxy or trifluoromethoxy
R⁷ represents hydrogen, fluorine, chlorine, methyl, difluoromethyl, trifluoromethyl, methoxy or trifluoromethoxy
with the proviso, that at least one or R⁶ and R⁷ is not hydrogen,
R^{7A} represents hydrogen, fluorine or chlorine.
R⁸ represents hydrogen or fluorine,
R^{8A} represents hydrogen or fluorine,
X represents CH or N,
Y represents CH or N,
Z represents CR⁹,
where at most one of X and Y is N,
R⁹ represents hydrogen or methyl,
A¹ and A² represent at the same time CH₂ or O, or
one of A¹ and A² represents O and the other represents NMe,
A³ represents CH₂,
R¹⁰ represents hydrogen,
R³ represents hydrogen, fluorine, chlorine or methyl,
R⁴ represents hydrogen, chlorine, methyl, isopropyl, trifluoromethyl, pentafluoroethyl, cyclopropyl, 2,2-difluorocyclopropan-1-yl, 3,3-difluorocyclobutan-1-yl, cyano or methoxymethyl,
and/or a salt thereof, N-oxide, solvate thereof and/or solvate of a salt and/or of an N-oxide thereof.

4. The compound according to claim 1, wherein
R¹ represents ethyl, propan-1-yl, propan-2-yl, 3,3,3-trifluoropropan-1-yl, 3,3-difluoropropan-1-yl, 1,1,1-trifluoropropan-2-yl, 4,4,4-trifluorobutan-1-yl, cyclobutyl or the group -L-R^{E},
where ethyl may be substituted by 1 cyclopropyl substituent,
and
where the propanyls may be substituted by 1 hydroxyl substituent,
and
where the di- and trifluoropropanyls and the trifluorobutanyl may be further substituted by 1 hydroxyl substituent,
and
where in the cyclobutyl ring one carbon is replaced by NR⁵, and where the cyclobutyl may be substituted by 1 substituent selected from the group consisting of methyl and phenyl,
where the phenyl may be substituted by 1 substituent selected from the group consisting of fluorine and methoxy,
and
R⁵ represents hydrogen or methyl,
and
L represents C₁-C₄-alkanediyl,
where alkanediyl may be substituted by 1 substituent selected from the group consisting of hydroxyl and 1-hydroxycyclopropyl, and additionally by up to 3 fluorine atoms,
R^{E} represents phenyl,
where phenyl may be substituted by 1 or 2 substituents selected from the group consisting of fluorine, chlorine, hydroxyl, methoxy, trifluoromethyl, trifluoromethoxy and difluoromethoxy,
R² represents 4-chlorophenyl, 3-chlorophenyl, 4-chloro-3-fluorophenyl, 3-chloro-4-fluorophenyl, 4-chloro-3-methylphenyl, 3-chloro-4-methylphenyl, 4-chloro-3-difluoromethyl-phenyl, 3-chloro-4-difluoromethyl-phenyl, 4-fluoro-3-methoxyphenyl, 3-fluoro-4-methoxyphenyl, 4-chloro-3-methoxyphenyl, 3-chloro-4-methoxyphenyl, 4-chloro-3-trifluoromethoxy-phenyl, 3-chloro-4-trifluoromethoxy-phenyl, 3,4-dichlorophenyl, 3,4-dimethylphenyl, 3-fluoro-4-methylphenyl, 4-fluoro-3-methylphenyl, 4-methyl-3-trifluoromethylphenyl, 3-methyl-4-trifluoromethylphenyl, 2,3-difluoro-4-methylphenyl, 2-fluoro-3,4-dimethylphenyl, 4-chloro-3,5-difluoropheynl, 4-chloro-2-fluoro-5-methyl, 4,5-dichloro-2-fluorophenyl, 4-chloro-2,5-difluorophenyl, or represents a group of formula 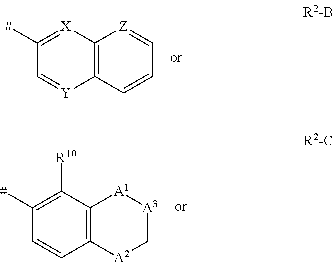
where # is the point of attachment to the pyrazinone ring,
X, Y and Z all represent CH or
X is CH, Y is N, Z is CR⁹ and R⁹ is methyl,
A¹ is O, A² is O, A³ is CH₂ and R¹⁰ is hydrogen,
R³ represents hydrogen, fluorine or methyl,
R⁴ represents hydrogen, methyl, isopropyl, trifluoromethyl or cyclopropyl,
and/or a salt thereof, N-oxide, solvate thereof and/or solvate of a salt and/or of an N-oxide thereof.
and the salts thereof, the N-oxides, the solvates thereof and the solvates of the salts or of the N-oxides thereof.

5. The compound according to claim 1, wherein
R¹ represents 1-cyclopropylethyl, 1-hydroxypropan-2-yl, 3,3,3-trifluoro-2-hydroxy-propan-1-yl, 3,3-difluoro-2-hydroxypropan-1-yl, 4,4,4-trifluoro-3-hydroxy-butan-1-yl, 3-(4-fluorophenyl)-1-methylazetidin-3-yl, 3-(4-methoxyphenyl)-1-methyl-azetidin-3-yl or the group -L-R^{E},
where
L represents an C₁-C₄-alkanediyl selected from the group consisting of 1-hydroxycyclopropyl-methan-1,1,-diyl [—CH(1-hydroxycyclopropyl)—], ethan-1,1-diyl [—CH(CH₃)—], 2-hydroxy-ethan-1,1-diyl [—CH(CH₂OH)—], 3-hydroxy-propan-1,1-diyl {—CH[(CH₂)₂OH]—}, 2-hydroxy-2-methylpropan-1,1-diyl {—CH[C(CH₃)₂OH]—} and 2,2-difluoro-3-hydroxy-propan-1,1-diyl [—CH(CF₂CH₂OH)—],
where the alkanediyl may be substituted by 1 hydroxyl substituent,
R^{E} represents phenyl,
where phenyl may be substituted in 4-position by fluorine, hydroxyl, methoxy or trifluoromethyl, or where phenyl may be disubstituted in 3- and 4-position by fluorine,
R² represents 4-chlorophenyl, 3-chlorophenyl, 4-chloro-3-fluorophenyl, 3-chloro-4-fluorophenyl, 4-chloro-3-methylphenyl, 3-chloro-4-methylphenyl, 3,4-dichlorophenyl, 3,4-dimethylphenyl, 3-fluoro-4-methylphenyl, 4-methyl-3-trifluoromethylphenyl, 3-methyl-4-trifluoromethylphenyl or 4-chloro-2,5-difluorophenyl,
R³ represents hydrogen,
R⁴ represents isopropyl, trifluoromethyl or cyclopropyl,
and/or a salt thereof, N-oxide, solvate thereof and/or solvate of a salt and/or of an N-oxide thereof.

6. A method for preparing a compound of formula (I) or a salt thereof, solvate thereof or solvate of a salt thereof according to claim 1, wherein
[A] one or more compounds of formula (II) 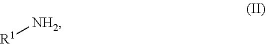
are reacted with one or more compounds of formula (III) 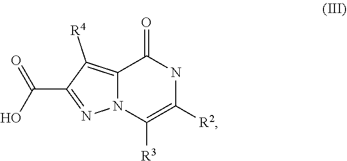
in the presence of a dehydrating agent to give a compound of formula (I).

7. The compound according to claim 1 for treatment and/or prophylaxis of one or more diseases.

8. The compound according to claim 1 for treatment and/or prophylaxis of one or more thrombotic or thromboembolic disorders, diabetes, urogenital and/or ophthalmic disorders.

9. A product comprising the compound according to claim 1 for treatment and/or prophylaxis of one or more diseases.

10. A product comprising the compound according to claim 1 for treatment and/or prophylaxis of one or more thrombotic or thromboembolic disorders, diabetes, urogenital and/or ophthalmic disorders.

11. A medicament comprising a compound according to claim 1 in combination with an inert, nontoxic, pharmaceutically suitable excipient.

12. The medicament according to claim 11 for treatment and/or prophylaxis of one or more thrombotic or thromboembolic disorders, diabetes, urogenital and/or ophthalmic disorders.

13. A method for treatment and/or prophylaxis of one or more thrombotic or thromboembolic disorders, diabetes, urogenital and/or ophthalmic disorders in humans and/or animals by administration of a therapeutically effective amount of at least one compound according to claim 1, or a medicament thereof.
